SEQUENCE LISTING (559 sequences)

SEQ ID NO: 1 - protein, 215 aa, Aeromonas salmonicida
MYRRHLKHSRVKNLFKFVSAKMNTVFTVESALEFDTCFHLEYSPSVKFYEAQPEGFYYEFAGRQCPYTPDFRLVDQNDSVSFLEIKPSDKVADPDFLHRFPLKQQRAIELSSPLKLVTEKQIRIAPILGNLKLLHRYSGFQSFTPLHMQLLGLVQKLGRVSLLRLSDSIDAPPEEVLASALSLIARGIMQSDLTVQKIGISSFVWAGGHSGIDHG

SEQ ID NO: 2 - protein, 624 aa, Aeromonas salmonicida
MDKHNGGLFEDEFVIPQPSTSTSPIDAIQAVLPATVDSFPYVLKVEALHRRDYILWVEKNLAGGWTEKNLTPLLADAALVLPPPTPNWRTLARWRKIYIQHGRKLVSLIPKHQAKGNARSRLPPSDELFFEQAVHRYLVGEQPSIASAFQLYSDSIRIENLGVVENPIKTISYMAFYNRIKKLPAYQVMKSRKGSYIADVEFKAIASHKPPSRIMERVEIDHTPLDLLLLDDDLLVPLGRPSLTLLIDAYSHCVVGFNLNFNQPSYESVRNALLSSISKKDYVKNKYPSIEHEWPCYGKPETLVVDNGVEFWSASLAQSCLELGINIQYNPVRKPWLKPMIERMFGIINRKLLEPIPGKTFSNIQEKGDYDPQKDAVMRFSTFLEIFHHWVIDVYHYEPDSRYRYIPIISWQHGNKDAPPAPIIGDDLTKLEVILSLSLHCTHRRGGIQRYHLRYDSDELASYRMNYPDQTRGKRKVLVKLNPRDISYVYVFLEDLGSYIRVPCIDPIGYTKGLSLQEHQINVKLHRDFINEQMDVVSLSKARIYLNDRIKNELIEVRRNIRQRNVKGVNKIAKYRNVGSHAETSIVHELNHPATNEVISKMESASQPEHCDDWDNFTSGLEPY

SEQ ID NO: 3 - protein, 882 aa, synthetic construct
MYRRHLKHSRVKNLFKFVSAKMNTVFTVESALEFDTCFHLEYSPSVKFYEAQPEGFYYEFAGRQCPYTPDFRLVDQNDSVSFLEIKPSDKVADPDFLHRFPLKQQRAIELSSPLKLVTEKQIRIAPILGNLKLLHRYSGFQSFTPLHMQLLGLVQKLGRVSLLRLSDSIDAPPEEVLASALSLIARGIMQSDLTVQKIGISSFVWAGGHSGIDHGGSGPKKKRKVGSGYPYDVPDYAYPYDVPDYAYPYDVPDYAGSGMDKHNGGLFEDEFVIPQPSTSTSPIDAIQAVLPATVDSFPYVLKVEALHRRDYILWVEKNLAGGWTEKNLTPLLADAALVLPPPTPNWRTLARWRKIYIQHGRKLVSLIPKHQAKGNARSRLPPSDELFFEQAVHRYLVGEQPSIASAFQLYSDSIRIENLGVVENPIKTISYMAFYNRIKKLPAYQVMKSRKGSYIADVEFKAIASHKPPSRIMERVEIDHTPLDLLLLDDDLLVPLGRPSLTLLIDAYSHCVVGFNLNFNQPSYESVRNALLSSISKKDYVKNKYPSIEHEWPCYGKPETLVVDNGVEFWSASLAQSCLELGINIQYNPVRKPWLKPMIERMFGIINRKLLEPIPGKTFSNIQEKGDYDPQKDAVMRFSTFLEIFHHWVIDVYHYEPDSRYRYIPIISWQHGNKDAPPAPIIGDDLTKLEVILSLSLHCTHRRGGIQRYHLRYDSDELASYRMNYPDQTRGKRKVLVKLNPRDISYVYVFLEDLGSYIRVPCIDPIGYTKGLSLQEHQINVKLHRDFINEQMDVVSLSKARIYLNDRIKNELIEVRRNIRQRNVKGVNKIAKYRNVGSHAETSIVHELNHPATNEVISKMESASQPEHCDDWDNFTSGLEPY

SEQ ID NO: 4 - protein, 335 aa, Aeromonas salmonicida
MDLSCHDADKLRSFIECYVETPLLRAIQEDFDRLRFNKQFAGEPQCMLLTGDTGTGKSSLIRHYAAKHPEQVRHGFIHKPLLVSRIPSRPTLESTMVELLKDLGQFGSSDRIHKSSAESLTEALIKCLKRCETELIIIDEFQELIENKTREKRNQIANRLKYISETAKIPIVLVGMPWATKIAEEPQWSSRLLIRRSIPYFKLSDDRENFIRLIMGLANRMPFETQARLETKHTIYALFAACYGSLRALKQLLDESVKQALAAHAETLKHEHIAVAYALFYPDQVNPFLQPIDEIKACEVKQYSRYEIDAAGKEEVLNPLQFTDKIPISQLLKKR

SEQ ID NO: 5 - protein, 350 aa, synthetic construct
MDLSCHDADKLRSFIECYVETPLLRAIQEDFDRLRFNKQFAGEPQCMLLTGDTGTGKSSLIRHYAAKHPEQVRHGFIHKPLLVSRIPSRPTLESTMVELLKDLGQFGSSDRIHKSSAESLTEALIKCLKRCETELIIIDEFQELIENKTREKRNQIANRLKYISETAKIPIVLVGMPWATKIAEEPQWSSRLLIRRSIPYFKLSDDRENFIRLIMGLANRMPFETQARLETKHTIYALFAACYGSLRALKQLLDESVKQALAAHAETLKHEHIAVAYALFYPDQVNPFLQPIDEIKACEVKQYSGSGPAAKKKKLDGSGRYEIDAAGKEEVLNPLQFTDKIPISQLLKKR

SEQ ID NO: 6 - protein, 410 aa, Aeromonas salmonicida
MHLLVRPEPFADEALESYFLRLSQENGFERYRIFSGSVQDWLHTTDHAAAGAFPLELSRLNIFHASRSSGLRVRALQLVDRLTDGAPFRLLQLALCHSAISFGNHYKAVHRSGVDIPLSFIRVHQIPCCPDCLRESAYVRQCWHFKPYVGCHRHGGRLIYSCPACGESLNYLASESINHCQCGFDLRTASTVPAQPDEIQLSALAYGCSFESSNPLLAIGSLSARFGALYWYQQRYLSDHEAVRDDRALTKAIGHFTAWPDAFWRELQQMVDDALVRQTKPLNHTDFVDVFGSVVADCRQIPMRNTGQNFILKNLIGFLTDLVARHPQCRVANVGDLLLSAVDAATLLSTSVEQVRRLHHEGFLPLSIRPASRNTVSPHRAVFHLRHVVELRQARMQSHHDHSSTYLPAW

SEQ ID NO: 7 - protein, 453 aa, synthetic construct
MPKKKRKVGSGEQKLISEEDLEQKLISEEDLEQKLISEEDLGSGHLLVRPEPFADEALESYFLRLSQENGFERYRIFSGSVQDWLHTTDHAAAGAFPLELSRLNIFHASRSSGLRVRALQLVDRLTDGAPFRLLQLALCHSAISFGNHYKAVHRSGVDIPLSFIRVHQIPCCPDCLRESAYVRQCWHFKPYVGCHRHGGRLIYSCPACGESLNYLASESINHCQCGFDLRTASTVPAQPDEIQLSALAYGCSFESSNPLLAIGSLSARFGALYWYQQRYLSDHEAVRDDRALTKAIGHFTAWPDAFWRELQQMVDDALVRQTKPLNHTDFVDVFGSVVADCRQIPMRNTGQNFILKNLIGFLTDLVARHPQCRVANVGDLLLSAVDAATLLSTSVEQVRRLHHEGFLPLSIRPASRNTVSPHRAVFHLRHVVELRQARMQSHHDHSSTYLPAW

SEQ ID NO: 8 - protein, 229 aa, Vibrio cholerae
MTSLPTPSAITTSALEYAFHTPARNLTKSRGKNIHRYVSVKMSKRITVESTLECDACYHFDFEPSIVRFCAQPIRFLYYLNGQSHSYVPDFLVQFDTNEFVLYEVKSAYAKNKPDFDVEWEAKVKAATELGLELELVEESDIRDTVVLNNLKRMHRYASKDELNNVHNSLLKIIKYNGAQSARCLGEQLGLKGRTVLPILCDLLSRCLLDTRLDKPLSLESRFELASYG

SEQ ID NO: 9 - protein, 603 aa, Vibrio cholerae
MAKKGFSSFHRKAVSSQDTLESIELVSSANCLESVTYQDISAFPETIAVEINFRLSILRFLARKCETIVAKSIEPHRVELQQNYSRKIPSAITIYRWWLAFRKSDYNPISLAPNIKDRGNRETKVSTVVDSIMEQAVERVISGRKVNVSSAYKRVRRKVRQYNLTHGTKYTYPKYESVRKRVKKKTPFELLAAGKGERVAKREFRRMGKKILTSSVLERVEIDHTVVDLFAVHEEYRIPLGRPWLTQLVDCYSKAVIGFYLGFEPPSYVSVSLALKNAIQRKDDLISSYESIENEWLCYGIPDLLVTDNGKEFLSKAFDQACESLLINVHQNKVETPDNKPHVERNYGTINTSLLDDLPGKSFSQYLQREGYDSVGEATLTLNEIREIYLIWLVDIYHKKPNQRGTNCPNVAWKKGCQEWEPEEFSGSKDELDFKFAIVDYKQLTKVGITVYKELSYSNDRLAEYRGKKGNHKVQFKYNPECMAVIWVLDEDMNEYFTVNAIDYEYASRVSLWQHKYNMKYQAELNSAEYDEDKEIDAEIKIEEIADRSIVKTNKIRARRRGARHQENSARAKSISNANPASIQKHEDEIVSADNDDWDIDYV

SEQ ID NO: 10 - protein, 874 aa, synthetic construct
MTSLPTPSAITTSALEYAFHTPARNLTKSRGKNIHRYVSVKMSKRITVESTLECDACYHFDFEPSIVRFCAQPIRFLYYLNGQSHSYVPDFLVQFDTNEFVLYEVKSAYAKNKPDFDVEWEAKVKAATELGLELELVEESDIRDTVVLNNLKRMHRYASKDELNNVHNSLLKIIKYNGAQSARCLGEQLGLKGRTVLPILCDLLSRCLLDTRLDKPLSLESRFELASYGGSGPKKKRKVGSGYPYDVPDYAYPYDVPDYAYPYDVPDYAGSGAKKGFSSFHRKAVSSQDTLESIELVSSANCLESVTYQDISAFPETIAVEINFRLSILRFLARKCETIVAKSIEPHRVELQQNYSRKIPSAITIYRWWLAFRKSDYNPISLAPNIKDRGNRETKVSTVVDSIMEQAVERVISGRKVNVSSAYKRVRRKVRQYNLTHGTKYTYPKYESVRKRVKKKTPFELLAAGKGERVAKREFRRMGKKILTSSVLERVEIDHTVVDLFAVHEEYRIPLGRPWLTQLVDCYSKAVIGFYLGFEPPSYVSVSLALKNAIQRKDDLISSYESIENEWLCYGIPDLLVTDNGKEFLSKAFDQACESLLINVHQNKVETPDNKPHVERNYGTINTSLLDDLPGKSFSQYLQREGYDSVGEATLTLNEIREIYLIWLVDIYHKKPNQRGTNCPNVAWKKGCQEWEPEEFSGSKDELDFKFAIVDYKQLTKVGITVYKELSYSNDRLAEYRGKKGNHKVQFKYNPECMAVIWVLDEDMNEYFTVNAIDYEYASRVSLWQHKYNMKYQAELNSAEYDEDKEIDAEIKIEEIADRSIVKTNKIRARRRGARHQENSARAKSISNANPASIQKHEDEIVSADNDDWDIDYV

SEQ ID NO: 11 - protein, 330 aa, Vibrio cholerae
MSETREARISRAKRAFVSTPSVRKILSYMDRCRDLSDLESEPTCMMVYGASGVGKTTVIKKYLNQNRRESEAGGDIIPVLHIELPDNAKPVDAARELLVEMGDPLALYETDLARLTKRLTELIPAVGVKLIIIDEFQHLVEERSNRVLTQVGNWLKMILNKTKCPIVIFGMPYSKVVLQANSQLHGRFSIQVELRPFSYQGGRGVFKTFLEYLDKALPFEKQAGLANESLQKKLYAFSQGNMRSLRNLIYQASIEAIDNQHETITEEDFVFASKLTSGDKPNSWKNPFEEGVEVTEDMLRPPPKDIGWEDYLRHSTPRVSKPGRNKNFFE

SEQ ID NO: 12 - protein, 345 aa, synthetic construct
MSETREARISRAKRAFVSTPSVRKILSYMDRCRDLSDLESEPTCMMVYGASGVGKTTVIKKYLNQNRRESEAGGDIIPVLHIELPDNAKPVDAARELLVEMGDPLALYETDLARLTKRLTELIPAVGVKLIIIDEFQHLVEERSNRVLTQVGNWLKMILNKTKCPIVIFGMPYSKVVLQANSQLHGRFSIQVELRPFSYQGGRGVFKTFLEYLDKALPFEKQAGLANESLQKKLYAFSQGNMRSLRNLIYQASIEAIDNQHETITEEDFVFASKLTSGDKPNSWKNPFEEGVEVTEDMLRPPPKDIGSGPAAKKKKLDGSGGWEDYLRHSTPRVSKPGRNKNFFE

SEQ ID NO: 13 - protein, 394 aa, Vibrio cholerae
MFLQRPKPYSDESLESFFIRVANKNGYGDVHRFLEATKRFLQDIDHNGYQTFPTDITRINPYSAKNSSSARTASFLKLAQLTFNEPPELLGLAINRTNMKYSPSTSAVVRGAEVFPRSLLRTHSIPCCPLCLRENGYASYLWHFQGYEYCHSHNVPLITTCSCGKEFDYRVSGLKGICCKCKEPITLTSRENGHEAACTVSNWLAGHESKPLPNLPKSYRWGLVHWWMGIKDSEFDHFSFVQFFSNWPRSFHSIIEDEVEFNLEHAVVSTSELRLKDLLGRLFFGSIRLPERNLQHNIILGELLCYLENRLWQDKGLIANLKMNALEATVMLNCSLDQIASMVEQRILKPNRKSKPNSPLDVTDYLFHFGDIFCLWLAEFQSDEFNRSFYVSRW

SEQ ID NO: 14 - protein, 437 aa, synthetic construct
MPKKKRKVGSGEQKLISEEDLEQKLISEEDLEQKLISEEDLGSGFLQRPKPYSDESLESFFIRVANKNGYGDVHRFLEATKRFLQDIDHNGYQTFPTDITRINPYSAKNSSSARTASFLKLAQLTFNEPPELLGLAINRTNMKYSPSTSAVVRGAEVFPRSLLRTHSIPCCPLCLRENGYASYLWHFQGYEYCHSHNVPLITTCSCGKEFDYRVSGLKGICCKCKEPITLTSRENGHEAACTVSNWLAGHESKPLPNLPKSYRWGLVHWWMGIKDSEFDHFSFVQFFSNWPRSFHSIIEDEVEFNLEHAVVSTSELRLKDLLGRLFFGSIRLPERNLQHNIILGELLCYLENRLWQDKGLIANLKMNALEATVMLNCSLDQIASMVEQRILKPNRKSKPNSPLDVTDYLFHFGDIFCLWLAEFQSDEFNRSFYVSRW

SEQ ID NO: 15 - protein, 207 aa, Vibrio cholerae
MFDQTKKSSHVHNICKFMSLKNDAVVRTLSILEFDFCFHLEYNPNIKSFTSQPFGFHYLFNNRKCRYTPDFLAIGHNEQSTFFEVKHSSQIPKPDFRERFEEKQRVALSEFNRRLVLVTEKQIRMGPTLDNFKLLHRYSGLRTVTEFQKRVLAFIQRKQMVKLQEVSLYFGLSEQDTLISTLPWISSGHVKTDLNTIGFGLETCVWC

SEQ ID NO: 16 - protein, 621 aa, Vibrio cholerae
MPPDSNSIFGFFDEFEASEEESQLLPKELILEPVEISSTIDSLPAKIQEEVLRRIKVITFVEKRLKGGWTEKNLNPILSLVESELQLTPPSWRTVATWKKSYAEAGREASALIPKHTFKGNRQKEMDSQSLIDEAIQNVYLTRERLSVAEAYRYYKSRVIQMNRGIVEGKIKPIAERSFYNRINELPPYEVAIARFGKRYADREYRSVGQQVVATKPMEFVEIDHTPVPVILIDDELDIPLGRPYLTMLYDRFSKCIVGCSINFREPSFDSVRKALLNSLLDKSWLKAKYPSIENEWPCHGKIDCLVVDNGAEFWSQSLEDSLRPLVSDIQYSQAAKPWRKSGIEKLFDQMNKGLVNALPGKTFTNPTQLQDYNPKKDAVVRVSVFLELLHKWIVDYYHMAPDSREREIPYHKWHQSKWTPSYYDGAEKEQLRVELGLLRHRTIGVAGIRLHNLRYQSAELIEYRKYCTPNNGKQLFVKTKTDPSDISYIHVYLESEKKYIKVPAVDNSGYTNGLSLFEHQRIQKVRRLNTKDLADDEALADTFLYMKKRIHEETDRFRRVKSSKPNLPKTGNTSRLAKFNDVGSEGPNSINVTPVRLKSEVVSDASEYLDDDDFEDIEGY

SEQ ID NO: 17 - protein, 873 aa, synthetic construct
MFDQTKKSSHVHNICKFMSLKNDAVVRTLSILEFDFCFHLEYNPNIKSFTSQPFGFHYLFNNRKCRYTPDFLAIGHNEQSTFFEVKHSSQIPKPDFRERFEEKQRVALSEFNRRLVLVTEKQIRMGPTLDNFKLLHRYSGLRTVTEFQKRVLAFIQRKQMVKLQEVSLYFGLSEQDTLISTLPWISSGHVKTDLNTIGFGLETCVWCGSGPKKKRKVGSGYPYDVPDYAYPYDVPDYAYPYDVPDYAGSGLTMPPDSNSIFGFFDEFEASEEESQLLPKELILEPVEISSTIDSLPAKIQEEVLRRIKVITFVEKRLKGGWTEKNLNPILSLVESELQLTPPSWRTVATWKKSYAEAGREASALIPKHTFKGNRQKEMDSQSLIDEAIQNVYLTRERLSVAEAYRYYKSRVIQMNRGIVEGKIKPIAERSFYNRINELPPYEVAIARFGKRYADREYRSVGQQVVATKPMEFVEIDHTPVPVILIDDELDIPLGRPYLTMLYDRFSKCIVGCSINFREPSFDSVRKALLNSLLDKSWLKAKYPSIENEWPCHGKIDCLVVDNGAEFWSQSLEDSLRPLVSDIQYSQAAKPWRKSGIEKLFDQMNKGLVNALPGKTFTNPTQLQDYNPKKDAVVRVSVFLELLHKWIVDYYHMAPDSREREIPYHKWHQSKWTPSYYDGAEKEQLRVELGLLRHRTIGVAGIRLHNLRYQSAELIEYRKYCTPNNGKQLFVKTKTDPSDISYIHVYLESEKKYIKVPAVDNSGYTNGLSLFEHQRIQKVRRLNTKDLADDEALADTFLYMKKRIHEETDRFRRVKSSKPNLPKTGNTSRLAKFNDVGSEGPNSINVTPVRLKSEVVSDASEYLDDDDFEDIEGY

SEQ ID NO: 18 - protein, 365 aa, Vibrio cholerae
MTMILKILKGISLNLTPKQLEQLKSFETCFIEYPAITEIYSIFDQLRFNHSLGGEPESFLLTGEAGSGKTALINNYLSRFQSGSTWGKQPVLSTRVPSRINEQNTLTQFLVDLDCKSGGRGIRRRNEIALGEAVVKQLKRKSVELIIVNEIQELVEFSTAEQRQVIANTFKYMSEEARVSFVLVGMPYADVIATEPQWNSRLSWRRKIDYFKLLKANSHSSKTASYGFDLEQKKHFARFVAGLSSRMGFDEPPVLTKNELLYPLFAMCRGECRALKHFLKDALLTSFNDNADTIDKAILSRTFAFKFPYLDNPFDRPLEQLSLHQIDSGSAYHLNAITTEDKIVAPRFTDAIPLSMLLSKNGLKA

SEQ ID NO: 19 - protein, 380 aa, synthetic construct
MTMILKILKGISLNLTPKQLEQLKSFETCFIEYPAITEIYSIFDQLRFNHSLGGEPESFLLTGEAGSGKTALINNYLSRFQSGSTWGKQPVLSTRVPSRINEQNTLTQFLVDLDCKSGGRGIRRRNEIALGEAVVKQLKRKSVELIIVNEIQELVEFSTAEQRQVIANTFKYMSEEARVSFVLVGMPYADVIATEPQWNSRLSWRRKIDYFKLLKANSHSSKTASYGFDLEQKKHFARFVAGLSSRMGFDEPPVLTKNELLYPLFAMCRGECRALKHFLKDALLTSFNDNADTIDKAILSRTFAFKFPYLDNPFDRPLEQLSLHQIDSGSGSGPAAKKKKLDGSGAYHLNAITTEDKIVAPRFTDAIPLSMLLSKNGLKA

SEQ ID NO: 20 - protein, 403 aa, Vibrio cholerae
MKTDIQHYSDESLESFLLRLSQEQGYERFSHFAEDIWFDTMEQHEAIAGAFPLELNRINIYHAQTTSQMRVRVLIHLENQLKLNNFGVLRLALSHSKAQFSPEYKAVHRLGSDYPFVFLGKRFTPICPLCISEAPYIRQQWQFLSQQACERHGCKLVHHCPECQSRLEYQTTESISQCECGFELRNSPVEDAPVAALLVARWLSGNDSKPLGLLKAEMTLSERYGFLLWYVNRYGDIENISFESFVEYCSCWPRVLKEELDELVNKADLIRIKDWKKTFFNEVFGALLKDCRQLPSRQLECNSVLTQVLAYFTKLMAAIPSSSKGNVGDVLLSPLEASTLLSCTTDEVYRLYEFGEIKAAIRPRMHTKIASHESAFTLRSVIETKLTRMCSENDGLSVYLPEW

SEQ ID NO: 21 - protein, 446 aa, synthetic construct
MPKKKRKVGSGEQKLISEEDLEQKLISEEDLEQKLISEEDLGSGKTDIQHYSDESLESFLLRLSQEQGYERFSHFAEDIWFDTMEQHEAIAGAFPLELNRINIYHAQTTSQMRVRVLIHLENQLKLNNFGVLRLALSHSKAQFSPEYKAVHRLGSDYPFVFLGKRFTPICPLCISEAPYIRQQWQFLSQQACERHGCKLVHHCPECQSRLEYQTTESISQCECGFELRNSPVEDAPVAALLVARWLSGNDSKPLGLLKAEMTLSERYGFLLWYVNRYGDIENISFESFVEYCSCWPRVLKEELDELVNKADLIRIKDWKKTFFNEVFGALLKDCRQLPSRQLECNSVLTQVLAYFTKLMAAIPSSSKGNVGDVLLSPLEASTLLSCTTDEVYRLYEFGEIKAAIRPRMHTKIASHESAFTLRSVIETKLTRMCSENDGLSVYLPEW

SEQ ID NO: 22 - protein, 209 aa, Aliivibrio wodanis
MYVRTLKQSQVKNISKFMSLKNDSIIRTESMLEFDMCFHLEYSPDVVSFESQPQGFHYEYQGKRLPYTPDFLITHSSGQQQLLEVKPLSKTQCPDFQSKFIQKQQAAQKLNLSLILITEKQIRTGHLLNNFKLLHRYAGLHSISATQKSIINLIQTVNKIQINQIAHRLNISNGEVLAGVLSWLSKGTLQTVYTNEMINGNSIVSLGLS

SEQ ID NO: 23 - protein, 620 aa, Aliivibrio wodanis
MDIEFPFTDEFQKILTTQSYPAEITNDKKTEVLTPSLDSYDDAIKAEVLRRISFLRWIKPRLKGGWTEKNLTPLLNDAEIDLKVSAPKWRTLAEWHKNYHKSGEKVSSLIPKHSHKGNKNMNTDSDFLITKAINEKYLTLNRCSISQTFKYYCDLVIIENRSIPTKKIKLVSQRTFYNRINALPKYDVALKRYGKRYADINYRTVDKMITATRPLERVEIDHTPLDLILLDDTLEIPLGRPYLTILIDSYSKCIVGYNLSFRPPSFESIRHAFCNACLDKSLITQQYPHLQHDWPVAGKIENLVVDNGAEFWSNSLEDSLLPFATNILYNKVGEPWMKPLVEKFFDLLNKGLVHSLPGTTRSRIEQLKGYNPKKDAAITFSLFLELFHTWIIDIYHMTSDTRETAVPYFKWQEGVTALPPLTYTDEEAQQLRIELGILNTRTVRLGGIFLHGLRYESEELSEYRKIWGAIDKNNLTLKTKTDPSDISHIFVYLTNESRYIKVPCITDISYTSGLTLFQHQTAQKLQRTKTRLQIDHEKLADSRMYVENRIAEEVEKIKSNKKRTAKTTHASKIARHQDIGSHTQKSIQVPNEQSEIKKLNKNEHDVLNGWDEQHDDLEGF

SEQ ID NO: 24 - protein, 869 aa, synthetic construct
MYVRTLKQSQVKNISKFMSLKNDSIIRTESMLEFDMCFHLEYSPDVVSFESQPQGFHYEYQGKRLPYTPDFLITHSSGQQQLLEVKPLSKTQCPDFQSKFIQKQQAAQKLNLSLILITEKQIRTGHLLNNFKLLHRYAGLHSISATQKSIINLIQTVNKIQINQIAHRLNISNGEVLAGVLSWLSKGTLQTVYTNEMINGNSIVSLGSGPKKKRKVGSGYPYDVPDYAYPYDVPDYAYPYDVPDYAGSGMDIEFPFTDEFQKILTTQSYPAEITNDKKTEVLTPSLDSYDDAIKAEVLRRISFLRWIKPRLKGGWTEKNLTPLLNDAEIDLKVSAPKWRTLAEWHKNYHKSGEKVSSLIPKHSHKGNKNMNTDSDFLITKAINEKYLTLNRCSISQTFKYYCDLVIIENRSIPTKKIKLVSQRTFYNRINALPKYDVALKRYGKRYADINYRTVDKMITATRPLERVEIDHTPLDLILLDDTLEIPLGRPYLTILIDSYSKCIVGYNLSFRPPSFESIRHAFCNACLDKSLITQQYPHLQHDWPVAGKIENLVVDNGAEFWSNSLEDSLLPFATNILYNKVGEPWMKPLVEKFFDLLNKGLVHSLPGTTRSRIEQLKGYNPKKDAAITFSLFLELFHTWIIDIYHMTSDTRETAVPYFKWQEGVTALPPLTYTDEEAQQLRIELGILNTRTVRLGGIFLHGLRYESEELSEYRKIWGAIDKNNLTLKTKTDPSDISHIFVYLTNESRYIKVPCITDISYTSGLTLFQHQTAQKLQRTKTRLQIDHEKLADSRMYVENRIAEEVEKIKSNKKRTAKTTHASKIARHQDIGSHTQKSIQVPNEQSEIKKLNKNEHDVLNGWDEQHDDLEGF

SEQ ID NO: 25 - protein, 336 aa, Aliivibrio wodanis
MHTLSSTQKEQLISFNQCFIEYPIITHIYSIFNDLRMNQGLGAEPQCMLLLGDTGSGKSALINNYLLRQPPSNFSALSSLPVLHTRIPRRVNNEQTMYQLLTDLGQSPSGSRRAKRSEIALAEGVVRALKRKKTELIIINEFQELIEFSSAKERQNVANTLKYISEEARVSIVLVGMPYADIIAEEPQWGSRLTWKTQIEYFSLKNDMKTYVQFLKGLANRMGYDEVPSLHSKELAIPLFSICRGELRQLKNFCSDAMLESFKQNKNTLTHDVLSATFKYKFPTKKNPFEMNVADVPIQEVESYSKYNLNAMTDDERLTATKFLDAMSLSSLLSKT

SEQ ID NO: 26 - protein, 351 aa, synthetic construct
MHTLSSTQKEQLISFNQCFIEYPIITHIYSIFNDLRMNQGLGAEPQCMLLLGDTGSGKSALINNYLLRQPPSNFSALSSLPVLHTRIPRRVNNEQTMYQLLTDLGQSPSGSRRAKRSEIALAEGVVRALKRKKTELIIINEFQELIEFSSAKERQNVANTLKYISEEARVSIVLVGMPYADIIAEEPQWGSRLTWKTQIEYFSLKNDMKTYVQFLKGLANRMGYDEVPSLHSKELAIPLFSICRGELRQLKNFCSDAMLESFKQNKNTLTHDVLSATFKYKFPTKKNPFEMNVADVPIQEVESYSGSGPAAKKKKLDGSGKYNLNAMTDDERLTATKFLDAMSLSSLLSKT

SEQ ID NO: 27 - protein, 399 aa, Aliivibrio wodanis
MLNPIELYEDESLESCLLRISQNNCYDSFQDFSDEVWFQVKEEDREVRGTFPATLNTVNIYHSHTSSDLKLKALIKIEQWLEINNSPLLKSALSRSSSTFLRQHSAVFRNGVDIPRILLRKNGIPVCPECLKENEYIRQEWHFITHDVCTRHKIGLLHHCPECKASINYQKIENITVCQCGFKFSDHLAPQANSNALLIAQWLNGENTKLANIWGEHQAISSRFGVLLWYINRYNLTDDFSTSFVKYSLNWPSNFYSELDEQIDKAKTVQIKPFNKIFFNEIFDNLLLDCQRLPTREFKTNPILSHVYQYFLSRYQIQPNSDVFSILLSPLEASSLLSCTTDQIYRLYELGFLKLGVRPKLHQKIASHQSVFTLSSIILVKLSNMQSSQDELHHYLSAW

SEQ ID NO: 28 - protein, 442 aa, synthetic construct
MPKKKRKVGSGEQKLISEEDLEQKLISEEDLEQKLISEEDLGSGLNPIELYEDESLESCLLRISQNNCYDSFQDFSDEVWFQVKEEDREVRGTFPATLNTVNIYHSHTSSDLKLKALIKIEQWLEINNSPLLKSALSRSSSTFLRQHSAVFRNGVDIPRILLRKNGIPVCPECLKENEYIRQEWHFITHDVCTRHKIGLLHHCPECKASINYQKIENITVCQCGFKFSDHLAPQANSNALLIAQWLNGENTKLANIWGEHQAISSRFGVLLWYINRYNLTDDFSTSFVKYSLNWPSNFYSELDEQIDKAKTVQIKPFNKIFFNEIFDNLLLDCQRLPTREFKTNPILSHVYQYFLSRYQIQPNSDVFSILLSPLEASSLLSCTTDQIYRLYELGFLKLGVRPKLHQKIASHQSVFTLSSIILVKLSNMQSSQDELHHYLSAW

SEQ ID NO: 29 - protein, 215 aa, Parashewanella spongiae
MPISRRNISHSRVKNLSKLSNFKNPNSEKRIAESHNEFLAAHFLNYFPIVKSFQFQPLAFDYENQDEIHSYTSDFLVELETGKFVYIEVKEEKALYSEDFKSIFEAKRAAARQLNKELILITENQYNIPPRIDNIKSLLNVGGFWADDNISNLVVGIVKKSETIDIEGIAYHLSQFSREEIFKAIRILILKREIYFDLSSSELTFDSKVSSDTTQ

SEQ ID NO: 30 - protein, 602 aa, Parashewanella spongiae
YNNAEFFIDEFVEFDFNKKPAKNEVKLFPKDMDIFPEKYKQEALAKKRYIKWVERKLVGKWTEKNINLLLHEIPNFGVNPTPCSRSIMRWKDAYTKGSRKLIALVPKHVSKGRTIAVSEHDEFIEHGISNYLTELRLSINECYKKYETQLRTNTDLEPVSYNTFKLRIDKLPKYDVKCAREGKAAADIDFNNYDEHCPPKRLYEQVEIDHTVLTVILLDSEYLFPIGRPTLTVLIDKLSHCICGFYVSYEPPSYNSARQAILHSIKPKDYIKNLYPSIKNEWNCHGKIENLIVDNGAEFWSTNLEVACENWMNIQFNPVGKPWKKAFVERFIGTTCREFTARFKGKTFSNILEKMKYDPKKDAVMRFDLFLELFHKWIIDDYHQRADSRFKYIPNELWQKNYLKSPVLKLDQAEEEKLENDFLCTEWREWRKGGIHIFNLRYDSEYLSKVRKQYVKEGNDKKQKILVKYSPENINTIRIYIEDLGKYIEVPCVDSVGYTKGLSLFNHQVNLRVHRTYIKSKIDVVSLAEVRKYVNDRVEEEEEFVEKGRKKNLSANKARSRYKSINSKNSISKKDNKFEDIEKSEDASPEDWNNFAEGLEGF

SEQ ID NO: 31 - protein, 860 aa, synthetic construct
MPISRRNISHSRVKNLSKLSNFKNPNSEKRIAESHNEFLAAHFLNYFPIVKSFQFQPLAFDYENQDEIHSYTSDFLVELETGKFVYIEVKEEKALYSEDFKSIFEAKRAAARQLNKELILITENQYNIPPRIDNIKSLLNVGGFWADDNISNLVVGIVKKSETIDIEGIAYHLSQFSREEIFKAIRILILKREIYFDLSSSELTFDSKVSSDTTQGSGPKKKRKVGSGYPYDVPDYAYPYDVPDYAYPYDVPDYAGSGYNNAEFFIDEFVEFDFNKKPAKNEVKLFPKDMDIFPEKYKQEALAKKRYIKWVERKLVGKWTEKNINLLLHEIPNFGVNPTPCSRSIMRWKDAYTKGSRKLIALVPKHVSKGRTIAVSEHDEFIEHGISNYLTELRLSINECYKKYETQLRTNTDLEPVSYNTFKLRIDKLPKYDVKCAREGKAAADIDFNNYDEHCPPKRLYEQVEIDHTVLTVILLDSEYLFPIGRPTLTVLIDKLSHCICGFYVSYEPPSYNSARQAILHSIKPKDYIKNLYPSIKNEWNCHGKIENLIVDNGAEFWSTNLEVACENWMNIQFNPVGKPWKKAFVERFIGTTCREFTARFKGKTFSNILEKMKYDPKKDAVMRFDLFLELFHKWIIDDYHQRADSRFKYIPNELWQKNYLKSPVLKLDQAEEEKLENDFLCTEWREWRKGGIHIFNLRYDSEYLSKVRKQYVKEGNDKKQKILVKYSPENINTIRIYIEDLGKYIEVPCVDSVGYTKGLSLFNHQVNLRVHRTYIKSKIDVVSLAEVRKYVNDRVEEEEEFVEKGRKKNLSANKARSRYKSINSKNSISKKDNKFEDIEKSEDASPEDWNNFAEGLEGF

SEQ ID NO: 32 - protein, 376 aa, Parashewanella spongiae
MARLSTEQCVLLKNFKNEFIPHAIAETIHDDFERLRENHRLGGEQLCMLIYGDEGSGKKSIIKAYEDKCKNEEVIDEGKFKVPVLFSEVKLPITVNSFFTQLLIDLGEFAGAYRKAEGKNKNKDMSKQLEDILKERLIKLETELIIIYKFELLLQFDKKMRIDELANQLKSMAQELGIPLVIIGMPCIKRLMLTSGWRSYIHICRLIPYFKLSNELEKAFYVKVIKGLSNRAQKLFSFAPKLEDKSISYPLFAVSSGCFRTIRNYTNKAVLLAVNEGAEELTIEHFSKVFERDNEYPLLGKVDSTNDDSMKKLNESIKDERDKRDSINPFKISVDKLMVNEVIDYATYKYDEESASEVKFDTRFADKISINDLLRK

SEQ ID NO: 33 - protein, 391 aa, synthetic construct
MARLSTEQCVLLKNFKNEFIPHAIAETIHDDFERLRENHRLGGEQLCMLIYGDEGSGKKSIIKAYEDKCKNEEVIDEGKFKVPVLFSEVKLPITVNSFFTQLLIDLGEFAGAYRKAEGKNKNKDMSKQLEDILKERLIKLETELIIIYKFELLLQFDKKMRIDELANQLKSMAQELGIPLVIIGMPCIKRLMLTSGWRSYIHICRLIPYFKLSNELEKAFYVKVIKGLSNRAQKLFSFAPKLEDKSISYPLFAVSSGCFRTIRNYTNKAVLLAVNEGAEELTIEHFSKVFERDNEYPLLGKVDSTNDDSMKKLNESIKDERDKRDSINPFKISVDKLMVNEVIDYAGSGPAAKKKKLDGSGTYKYDEESASEVKFDTRFADKISINDLLRK

SEQ ID NO: 34 - protein, 397 aa, Parashewanella spongiae
MRFTVQTELFKDESLESYLLRLAVDNTYIDYSEFADVIGRWLVDHDHELEGAFPCSLDLVNLYHAKDSSIFRVRALKLFETLTSFKPSTLLSQSLLRTNYKFAQYTALKFGSSLIPRVMLRENKAPIPICPQCIKESAYIRQCWHLKPYTFCHKHNLRLLNECPKCGDEINYIRYEVIEKCICGADLSKMAAVHGDIKYQKCIKNLFNEIEGDKSSEIGKLLWFSKYKNIELDDTELLNEFYDYFEFWPATYLSELEQFELGGINKQIRPFNQTPVNDIWKEQIALSKLASPFKQNNEVLKVLSEYFVDLVYRYPKSETLNPADTLLTKLEASILLRTPLEQVNRLLNENYLHRAIKPKKHEIIEPFKPLLYLRQVIELMEVRGINQAYSNLYTTTW

SEQ ID NO: 35 - protein, 440 aa, synthetic construct
MPKKKRKVGSGEQKLISEEDLEQKLISEEDLEQKLISEEDLGSGRFTVQTELFKDESLESYLLRLAVDNTYIDYSEFADVIGRWLVDHDHELEGAFPCSLDLVNLYHAKDSSIFRVRALKLFETLTSFKPSTLLSQSLLRTNYKFAQYTALKFGSSLIPRVMLRENKAPIPICPQCIKESAYIRQCWHLKPYTFCHKHNLRLLNECPKCGDEINYIRYEVIEKCICGADLSKMAAVHGDIKYQKCIKNLFNEIEGDKSSEIGKLLWFSKYKNIELDDTELLNEFYDYFEFWPATYLSELEQFELGGINKQIRPFNQTPVNDIWKEQIALSKLASPFKQNNEVLKVLSEYFVDLVYRYPKSETLNPADTLLTKLEASILLRTPLEQVNRLLNENYLHRAIKPKKHEIIEPFKPLLYLRQVIELMEVRGINQAYSNLYTTTW

SEQ ID NO: 36 - protein, 209 aa, Pseudoalteromonas sp.
MYRRKLKHSRVKNLHKFASQKNKSTCLVESSLEFDACFHFEFSPSIAAFEAQPLGYEYEFDNRICRYTPDFLLTHTDGTQKFIEVKPQSKIADEDFRARFIEKQTIAKQDGRDLILVTDKQIRVYPTLNNLKLLHRYSGFQSLTELQASVLELVKQYGSIKVGQLVNFLKVTAGELLATVLRLLSLGQLFADLTTNEISIETAIWSNNV

SEQ ID NO: 37 - protein, 607 aa, Pseudoalteromonas sp.
MFNDGLFDDEFNQPLPKVETKLPQNYAKDLQALPEKIKNTTFAKLKYIQWLEANIQGGWTQKNLEPLLKVMPEVEGEKKPSWRTAARWYSAYTNADKNIMALIPSHQKKGNRERDTATDKFFEKALERYLVKEKPSVASAYKYYADLVIIENDSVVGSVLKPLTYKAFKNRIDNLPQYDVMVSRYGKRLADIAFNKVEGHTRPTRVLEKVEIDHTPLDLILLDDELHIPLGRPTLTMLVDVYSHCIVGFYFSFSEPSYDAVRRAMLNAMKPKSDVAKLYPDTINEWKCAGKIETLVVDNGAEFWSNSLELACEEIGINTQYNPVAKPWLKPFVERMFGTINTELLDPVPGKTFSNILQKHEYNPKKDAIMRFTTFMQLFHKWVVDVYHQDADSRFKYIPSQLWEQGFNTLPPTVLSNADLQQLDVVLSISNHRVLRKGGIRLENLSYDSTELANYRKQFSHKVSQEVLIKLNPDDISYIYVYLDKLEHYIKVPCIDPNGYTQNLSLNQHKINIRIHRDFISGSIDNVGLAKARMFIHNKIQNEFEELKNAPKHSKVKGGKALAKHQNVSSDSQKSIAQSEPLEPKKVTPKEQPTDSWDDFISDLDGF

SEQ ID NO: 38 - protein, 858 aa, synthetic construct
MYRRKLKHSRVKNLHKFASQKNKSTCLVESSLEFDACFHFEFSPSIAAFEAQPLGYEYEFDNRICRYTPDFLLTHTDGTQKFIEVKPQSKIADEDFRARFIEKQTIAKQDGRDLILVTDKQIRVYPTLNNLKLLHRYSGFQSLTELQASVLELVKQYGSIKVGQLVNFLKVTAGELLATVLRLLSLGQLFADLTTNEISIETAIWSNNVGSGPKKKRKVGSGYPYDVPDYAYPYDVPDYAYPYDVPDYAGSGFNDGLFDDEFNQPLPKVETKLPQNYAKDLQALPEKIKNTTFAKLKYIQWLEANIQGGWTQKNLEPLLKVMPEVEGEKKPSWRTAARWYSAYTNADKNIMALIPSHQKKGNRERDTATDKFFEKALERYLVKEKPSVASAYKYYADLVIIENDSVVGSVLKPLTYKAFKNRIDNLPQYDVMVSRYGKRLADIAFNKVEGHTRPTRVLEKVEIDHTPLDLILLDDELHIPLGRPTLTMLVDVYSHCIVGFYFSFSEPSYDAVRRAMLNAMKPKSDVAKLYPDTINEWKCAGKIETLVVDNGAEFWSNSLELACEEIGINTQYNPVAKPWLKPFVERMFGTINTELLDPVPGKTFSNILQKHEYNPKKDAIMRFTTFMQLFHKWVVDVYHQDADSRFKYIPSQLWEQGFNTLPPTVLSNADLQQLDVVLSISNHRVLRKGGIRLENLSYDSTELANYRKQFSHKVSQEVLIKLNPDDISYIYVYLDKLEHYIKVPCIDPNGYTQNLSLNQHKINIRIHRDFISGSIDNVGLAKARMFIHNKIQNEFEELKNAPKHSKVKGGKALAKHQNVSSDSQKSIAQSEPLEPKKVTPKEQPTDSWDDFISDLDGF

SEQ ID NO: 39 - protein, 333 aa, Pseudoalteromonas sp.
MLTDKQKAKLNEFRDVFIEYPIITTVFNDFDRLRLGKGLAGEKPCMLLNGDTGTGKTALIKQYKERHLPQFINGVMNHPVLVSRIPSNPTLESTLAELLKDLGQVGSTERKLRINGTRLTTSLIKCLKTCGTELIIIDEFQELIEHNQGKKRREIANRLKYINDEAGVSIVLVGMPWAEKIADEPQWSSRLLVRRQLPYFKLSENPKHFVQLIIGLANRMPFTEKPKLSEQATVFALFSLSKGCFRTLKYFLDDAVLYALMDNAKTLTTKHLVKAFGVLFPDVPNLFTLPVAEITASEVERYSLYKLESAQDEDPFIATKFTDQMPISQLLRK

SEQ ID NO: 40 - protein, 348 aa, synthetic construct
MLTDKQKAKLNEFRDVFIEYPIITTVFNDFDRLRLGKGLAGEKPCMLLNGDTGTGKTALIKQYKERHLPQFINGVMNHPVLVSRIPSNPTLESTLAELLKDLGQVGSTERKLRINGTRLTTSLIKCLKTCGTELIIIDEFQELIEHNQGKKRREIANRLKYINDEAGVSIVLVGMPWAEKIADEPQWSSRLLVRRQLPYFKLSENPKHFVQLIIGLANRMPFTEKPKLSEQATVFALFSLSKGCFRTLKYFLDDAVLYALMDNAKTLTTKHLVKAFGVLFPDVPNLFTLPVAEITASEVERYSGSGPAAKKKKLDGSGLYKLESAQDEDPFIATKFTDQMPISQLLRK

SEQ ID NO: 41 - protein, 391 aa, Pseudoalteromonas sp.
MHFLVQTKLYPDEALESYLLRLARDNSYDGYSELADILWQWLVEQDHDLEGALPLELGKVDVYHARQASSFRIRALKLVAQLADVNAGNILALAWRRSNFKFGNLVAVSRNEQTIPLELLRTDNIPVCIECLFESSYVPFHWHLKPYKTCHKHKSQLTTHCKECHNLIDYRASEEFLECSCGCKLTNSEQLNDADFKIAFALASSNSHKIVGLISWFAKVKQLDVSDADFNRTFVDYFSTWPESLTTELDLLTNNARLKQLNPFNKTKFNSVYGNVIRDGQIAATSNRKNKVLDELIKYFVELVDSNPKTKHPNIADLLLCTFDTAVLLNTTTEQVYRLHQEGFLNCAYPQKKHEQLRADSHVFYLRQVIELQQAFAAETPQTKKQFIAPW

SEQ ID NO: 42 - protein, 434 aa, synthetic construct
MPKKKRKVGSGEQKLISEEDLEQKLISEEDLEQKLISEEDLGSGHFLVQTKLYPDEALESYLLRLARDNSYDGYSELADILWQWLVEQDHDLEGALPLELGKVDVYHARQASSFRIRALKLVAQLADVNAGNILALAWRRSNFKFGNLVAVSRNEQTIPLELLRTDNIPVCIECLFESSYVPFHWHLKPYKTCHKHKSQLTTHCKECHNLIDYRASEEFLECSCGCKLTNSEQLNDADFKIAFALASSNSHKIVGLISWFAKVKQLDVSDADFNRTFVDYFSTWPESLTTELDLLTNNARLKQLNPFNKTKFNSVYGNVIRDGQIAATSNRKNKVLDELIKYFVELVDSNPKTKHPNIADLLLCTFDTAVLLNTTTEQVYRLHQEGFLNCAYPQKKHEQLRADSHVFYLRQVIELQQAFAAETPQTKKQFIAPW

SEQ ID NO: 43 - protein, 207 aa, Vibrio diazotrophicus
MYVRNLRKPSANKNVYKFVSVKNGCNIMCESSLEYDCCYYLEYSDDVVRYQSQPKGYRFPYQGKEHPYTPDFLVHKKDGTSYLLEVKPLSKTFSSEFQDVFRQKQIMASELGAPLLLVTDRQIRNDVHLNNLKLVHRYSGCIGNSSHLESVWSAVNQSSSICIKALSAILNLTIGEVFASVLRLIGLGKAKTKLDVLLDENSLISVA

SEQ ID NO: 44 - protein, 635 aa, Vibrio diazotrophicus
MSFGPFEDEFGSITNDVQQQYEASPEAKLSRLKYSPLETTKVIERDLSSFPEEQKLKALERYKLISLIAKEINGGWTPKNLIPLIDKHIETLNIPKPSDRTVKRWYKAFCESDGDIKSLVDSHHLKGNRQPRIEDDEPFFIEAVERFLDAVRPSYSKAYQVYCDRIEIENSTIVSGKIAKVSYEAFKKRLKKLPPYTVALKRHGKYYADKLFNYYEAVKMPTRILERVEIDHTPLDLILLDDELLVPLGRAYLTLLVDVFSGCIIGFHLGFKAPSYTAVSKAIIHSVKSKEYVNELPIGLSNQWICHGKIENLVVDNGAEFWSKSLDQACIEAGINIIYNKVRKPWLKPFVERKFGELIQGIVGWVPGRTFSNVLEKEDYDPQKDAVMRFSVFVEELHRWIIDVHNASADSRHTRIPNYHWQKSEEVLPPPALTERDEIQFRVIMGMVHKGALTSKGIKFKHLMYDNVALEHYRKQYPQSKDSRIKTVKIDPDDLSRIFVFLEEKKGYIEVPCKYDPLGYTKKLSLCEHLRTVKVHRDFIKGQVDSLSLAKARQALHERIKQEHENLRQMSLPHRAKKAKNGKKMAELAGVNSDSPKSITTDYPIEDTIQLHESTPVDDLQSLWNKRRALRKSGK

SEQ ID NO: 45 - protein, 634 aa, synthetic construct
SFGPFEDEFGSITNDVQQQYEASPEAKLSRLKYSPLETTKVIERDLSSFPEEQKLKALERYKLISLIAKEINGGWTPKNLIPLIDKHIETLNIPKPSDRTVKRWYKAFCESDGDIKSLVDSHHLKGNRQPRIEDDEPFFIEAVERFLDAVRPSYSKAYQVYCDRIEIENSTIVSGKIAKVSYEAFKKRLKKLPPYTVALKRHGKYYADKLFNYYEAVKMPTRILERVEIDHTPLDLILLDDELLVPLGRAYLTLLVDVFSGCIIGFHLGFKAPSYTAVSKAIIHSVKSKEYVNELPIGLSNQWICHGKIENLVVDNGAEFWSKSLDQACIEAGINIIYNKVRKPWLKPFVERKFGELIQGIVGWVPGRTFSNVLEKEDYDPQKDAVMRFSVFVEELHRWIIDVHNASADSRHTRIPNYHWQKSEEVLPPPALTERDEIQFRVIMGMVHKGALTSKGIKFKHLMYDNVALEHYRKQYPQSKDSRIKTVKIDPDDLSRIFVFLEEKKGYIEVPCKYDPLGYTKKLSLCEHLRTVKVHRDFIKGQVDSLSLAKARQALHERIKQEHENLRQMSLPHRAKKAKNGKKMAELAGVNSDSPKSITTDYPIEDTIQLHESTPVDDLQSLWNKRRALRKSGK

SEQ ID NO: 46 - protein, 328 aa, Vibrio diazotrophicus
MTSLQPTNNDVDVLLAEFHQSFVVYPDVEKVFEGLDWIVRRSQFGKFAPSMLITGGTGAGKTSVVEIYLNNHFSSSEVLITRVRPSFVETLIWAIEKLNVPYNSRSKRSEIGLQDYFINSVKKSKLKLLVIEEAQELFECASPKERQKIRDRLKMISDECRLPIVFIGIPTAKLILEDSQWDRRIMVKRDLPYIRITNEESLDVYIALLEGLEKTLPISVAPELTDMDMAMRLLAASRGMLGLIKELVGYAFELALLEGKRQITQNEFVQAFKSIFGPDISNPFEIELDKLLIPQIIEYEGYLLDSDSGDIKFTHQIFEDIPLTELLR

SEQ ID NO: 47 - protein, 343 aa, synthetic construct
MTSLQPTNNDVDVLLAEFHQSFVVYPDVEKVFEGLDWIVRRSQFGKFAPSMLITGGTGAGKTSVVEIYLNNHFSSSEVLITRVRPSFVETLIWAIEKLNVPYNSRSKRSEIGLQDYFINSVKKSKLKLLVIEEAQELFECASPKERQKIRDRLKMISDECRLPIVFIGIPTAKLILEDSQWDRRIMVKRDLPYIRITNEESLDVYIALLEGLEKTLPISVAPELTDMDMAMRLLAASRGMLGLIKELVGYAFELALLEGKRQITQNEFVQAFKSIFGPDISNPFEIELDKLLIPQIIEYEGSGPAAKKKKLDGSGGYLLDSDSGDIKFTHQIFEDIPLTELLR

SEQ ID NO: 48 - protein, 403 aa, Vibrio diazotrophicus
MDTDIEVYSDESLESFLLRLSKFQGYERFAHFAEDIWQTTLLQHEAIPGAFPFELSRINIYKAQTTSQMRVRVLIDLEKRLKFNDFGVLRLSLAHSKASFSPDYKAVNRYGADYPQAFLRKNFTPVCPKCLDEAAYIRQLWHFIPYQVCHKHHSQLAQRCPECGKLLNYQSSELIENCECGFSLLNGESEKESCSTLFVAQWLAGEKPVESGLMSQELTQSSRFGFLLWYINRYGELDDISFDGFVECCKSWPNKLNTDLDSIVQKADIVRIQPWNKIYFSEVFGDLLKECRSLPSRDLSKNPVLKNVVLYFRALITNNPKVKSANIGDVLLSPLEASTLLSCTTDEIYRLYQFGQLKAQHTPKLQSKIENHHSVFTLRSIIELKLSSMCSETDGLNHYLPEW

SEQ ID NO: 49 - protein, 446 aa, synthetic construct
MPKKKRKVGSGEQKLISEEDLEQKLISEEDLEQKLISEEDLGSGDTDIEVYSDESLESFLLRLSKFQGYERFAHFAEDIWQTTLLQHEAIPGAFPFELSRINIYKAQTTSQMRVRVLIDLEKRLKFNDFGVLRLSLAHSKASFSPDYKAVNRYGADYPQAFLRKNFTPVCPKCLDEAAYIRQLWHFIPYQVCHKHHSQLAQRCPECGKLLNYQSSELIENCECGFSLLNGESEKESCSTLFVAQWLAGEKPVESGLMSQELTQSSRFGFLLWYINRYGELDDISFDGFVECCKSWPNKLNTDLDSIVQKADIVRIQPWNKIYFSEVFGDLLKECRSLPSRDLSKNPVLKNVVLYFRALITNNPKVKSANIGDVLLSPLEASTLLSCTTDEIYRLYQFGQLKAQHTPKLQSKIENHHSVFTLRSIIELKLSSMCSETDGLNHYLPEW

SEQ ID NO: 50 - protein, 209 aa, Shewanella sp.
MYIRNLRKPSPNKNVFKFASAKVSETIMCESTLEFDACFHHEYNETIETFGSQPKGFYYCFEGKRLPYTPDALLHYIDGTTKFHEYKPYSKTFDPIFRAKFVAKKEAAQALGTELILVTDKQIRVNPILNNLKLLHRYSGIYGVTDIQRELLQLIRHSGKIQLDDVADEYELSVGETRSFLYSLINKGLLEADLTQDDLSCNPFVWCNA

SEQ ID NO: 51 - protein, 617 aa, Shewanella sp.
MIEFKDEFTESTSVKKPDTPGQYIKLDDAEILKRDLDTFPDFLKEKAFDKYKLISFIEQENSGGWTQKKLDPILDKLFEGNRDKRPNWRTVVRWRKSYIDSNGDLASLVVKRHKMGNRKKRVEGDEVFFERALSRFLDAKRPKVTTAYQYYKDAITIENETIVDGEIPIISYTAFNQRIKSLPPYPIAVARHGKFKADQWFAYCSSHIPPTRILERVEIDHTPLDLILLDDELQLPLGRPYLTLIVDVFSNCVLGFHLSYKAPSYVSAAKAIVHAIKPKTLGIVGIELQNDWPCYGKFETLVVDNGAEFWSKSLDHACKEAGINIQYNPVRKPWLKPFVERFFGMINQYFLTELPGKTFSNILEKEDYKPEKDAIMRFSVFVEEFHRWIVDIYHQDSDSRDTRIPIKQWQHGFDVYPPLQMSVEDEKRFNVLMGITDERTLTRNGFKFEELMYDSTALADYRKHYPQTKDTIKKLIKIDPDDLSNIHVYLEELEGYLKVPCTDTTGYANGLSLHEHKVIKKINREIIRESKDNLGLAKARMAIHARVQQEQELFNESKTKAKISAVKKQAQLADISNTGQGTIRLENSDTLSDITNKPESNISDILDNWDDNIEGFE

SEQ ID NO: 52 - protein, 868 aa, synthetic construct
MYIRNLRKPSPNKNVFKFASAKVSETIMCESTLEFDACFHHEYNETIETFGSQPKGFYYCFEGKRLPYTPDALLHYIDGTTKFHEYKPYSKTFDPIFRAKFVAKKEAAQALGTELILVTDKQIRVNPILNNLKLLHRYSGIYGVTDIQRELLQLIRHSGKIQLDDVADEYELSVGETRSFLYSLINKGLLEADLTQDDLSCNPFVWCNAGSGPKKKRKVGSGYPYDVPDYAYPYDVPDYAYPYDVPDYAGSGIEFKDEFTESTSVKKPDTPGQYIKLDDAEILKRDLDTFPDFLKEKAFDKYKLISFIEQENSGGWTQKKLDPILDKLFEGNRDKRPNWRTVVRWRKSYIDSNGDLASLVVKRHKMGNRKKRVEGDEVFFERALSRFLDAKRPKVTTAYQYYKDAITIENETIVDGEIPIISYTAFNQRIKSLPPYPIAVARHGKFKADQWFAYCSSHIPPTRILERVEIDHTPLDLILLDDELQLPLGRPYLTLIVDVFSNCVLGFHLSYKAPSYVSAAKAIVHAIKPKTLGIVGIELQNDWPCYGKFETLVVDNGAEFWSKSLDHACKEAGINIQYNPVRKPWLKPFVERFFGMINQYFLTELPGKTFSNILEKEDYKPEKDAIMRFSVFVEEFHRWIVDIYHQDSDSRDTRIPIKQWQHGFDVYPPLQMSVEDEKRFNVLMGITDERTLTRNGFKFEELMYDSTALADYRKHYPQTKDTIKKLIKIDPDDLSNIHVYLEELEGYLKVPCTDTTGYANGLSLHEHKVIKKINREIIRESKDNLGLAKARMAIHARVQQEQELFNESKTKAKISAVKKQAQLADISNTGQGTIRLENSDTLSDITNKPESNISDILDNWDDNIEGFE

SEQ ID NO: 53 - protein, 343 aa, Shewanella sp.
MNTLTAHQMEQLGRFNDCFVMHPQAKVIFNDFDDLRLNRNFQSDQQCMLLTGDTGVGKSHLINNYKKRVLASQTYSRTSMPVLVTRISSHKGLDATLRQMLTDLESFGSQQRKGQNYKIDLKTQLVKNLVRANVELLIFNEFQELIEFKTPKERQTIANELKFISEEARVPIVLVGMPWTEQIAEEPQWSSRLIRRRKLEYFSLQKDSKYYRQYLIGLAKHMPFDEPPKIEDKHIAIPLFAACRGESRVLNHLLSETLKLVMVNGDRSLDIRHLAQTYRKLYESQESEAASVFFNPFLEPLDKVLISEVVKPSRYNPNAMTPDEMLIKREFSAPSTLAQLLSK

SEQ ID NO: 54 - protein, 358 aa, synthetic construct
MNTLTAHQMEQLGRFNDCFVMHPQAKVIFNDFDDLRLNRNFQSDQQCMLLTGDTGVGKSHLINNYKKRVLASQTYSRTSMPVLVTRISSHKGLDATLRQMLTDLESFGSQQRKGQNYKIDLKTQLVKNLVRANVELLIFNEFQELIEFKTPKERQTIANELKFISEEARVPIVLVGMPWTEQIAEEPQWSSRLIRRRKLEYFSLQKDSKYYRQYLIGLAKHMPFDEPPKIEDKHIAIPLFAACRGESRVLNHLLSETLKLVMVNGDRSLDIRHLAQTYRKLYESQESEAASVFFNPFLEPLDKVLISEVVKPSGSGPAAKKKKLDGSGRYNPNAMTPDEMLIKREFSAPSTLAQLLSK

SEQ ID NO: 55 - protein, 411 aa, Shewanella sp.
MAFLFSPKARSFSDESLESYLLRVVAENFFDSYQQLSLAIREELHELDFEAHGAFPVELKRLNVYHAKHNSHFRMRALGLLESLLDLPPHELQKLALLRSNKRFVGGMSAVHRNGVDIPLSFIRCADEDGIESLPICPQCLKEEPYIRQAWHIKPIEVCAKHECELIHHCPDCQQPISYIENESITHCSCGFEFATASSEKADSQAVVLSRSLFDGDALSNNPLLFMGTSVTHRFAALLWYLKRHVQNIECKLDESVNYFEAWPENFYQELDELLAGAELKLIDLFNRTSLSFIFGELILQSQCLLPEDKTPHFIDMGLMEYLGKLVESHPKSKKPNVADMLVSVTETAVLLSTSHEQVYRLYQDGVLTAGFKQKIRTRIDPHIGVFYLRQVIEYKTSFGNDKQGMYLSAW

SEQ ID NO: 56 - protein, 454 aa, synthetic construct
MPKKKRKVGSGEQKLISEEDLEQKLISEEDLEQKLISEEDLGSGAFLFSPKARSFSDESLESYLLRVVAENFFDSYQQLSLAIREELHELDFEAHGAFPVELKRLNVYHAKHNSHFRMRALGLLESLLDLPPHELQKLALLRSNKRFVGGMSAVHRNGVDIPLSFIRCADEDGIESLPICPQCLKEEPYIRQAWHIKPIEVCAKHECELIHHCPDCQQPISYIENESITHCSCGFEFATASSEKADSQAVVLSRSLFDGDALSNNPLLFMGTSVTHRFAALLWYLKRHVQNIECKLDESVNYFEAWPENFYQELDELLAGAELKLIDLFNRTSLSFIFGELILQSQCLLPEDKTPHFIDMGLMEYLGKLVESHPKSKKPNVADMLVSVTETAVLLSTSHEQVYRLYQDGVLTAGFKQKIRTRIDPHIGVFYLRQVIEYKTSFGNDKQGMYLSAW

SEQ ID NO: 57 - protein, 209 aa, Pseudoalteromonas sp.
MYIRNLRKPSPNKNVFKFASTKVSSVVMCESSLEFDACFHHEYNDLIESFGSQPEGFKYEFMGKSLPYTPDALISYTDKTQKYHEYKPYSKIASPLFRAEFAAKRAASLKLGIDLVLVTDRQIRVNPILNNLKLLHRYSGVYGISGIQKELLSFIHKSGVIKLNDISSQVGIPIGETRSFLFGLMHKGLVKADLGCDDLTNNPTLWATP

SEQ ID NO: 58 - protein, 613 aa, Pseudoalteromonas sp.
MTDFFNEFDESLVPLKPQTPTQYVKLDDANLIQRDLDTFSDTFKNQALQRYKLISTIDKKLSRGWTQRNLDPILDELFKGGDVVRPNWRTVARWRKKYIESNGDIASLADKNHKMGNRTNRIKGDDKFFDKALERFLDAKRPTIATAYQYYKDLIVIENESIVEGKIPIISYNAFNKRIKAIPPYAVAVARHGKFKADQWFAYCAAHVPPTRILERVEIDHTPLDLILLDDELLIPIGRPYLTLLIDVFSGCVLGFHLSYKSPSYVSAAKAITHAIKPKSLDALNIELQNDWPCFGKFENLVVDNGAEFWSKNLEHACQSAGINIQYNPVRKPWLKPFIERFFGVMNEYFLPELPGKTFSNILEKEEYKPEKDAIMRFSTFVEEFHRWIADVYHQDSNSRETRIPIKRWQQGFDAYPPLTMNEEEETRFSMLMRISDSRTLTRNGFKYQELMYDSTALADYRKHYPQTKETVKKLIKVDPDDISKIYVYLEELESYLEVPCTDPTGYTDGLSIYEHKTIKKINREVIRESKDSLGLAKARMAIHERVKQEQEVFIESKTKAKITAVKKQAQIADVSNTGTSTIKVSEESAAPVQKHISNDNSDDWDDDLEAFE

SEQ ID NO: 59 - protein, 864 aa, synthetic construct
MYIRNLRKPSPNKNVFKFASTKVSSVVMCESSLEFDACFHHEYNDLIESFGSQPEGFKYEFMGKSLPYTPDALISYTDKTQKYHEYKPYSKIASPLFRAEFAAKRAASLKLGIDLVLVTDRQIRVNPILNNLKLLHRYSGVYGISGIQKELLSFIHKSGVIKLNDISSQVGIPIGETRSFLFGLMHKGLVKADLGCDDLTNNPTLWATPGSGPKKKRKVGSGYPYDVPDYAYPYDVPDYAYPYDVPDYAGSGTDFFNEFDESLVPLKPQTPTQYVKLDDANLIQRDLDTFSDTFKNQALQRYKLISTIDKKLSRGWTQRNLDPILDELFKGGDVVRPNWRTVARWRKKYIESNGDIASLADKNHKMGNRTNRIKGDDKFFDKALERFLDAKRPTIATAYQYYKDLIVIENESIVEGKIPIISYNAFNKRIKAIPPYAVAVARHGKFKADQWFAYCAAHVPPTRILERVEIDHTPLDLILLDDELLIPIGRPYLTLLIDVFSGCVLGFHLSYKSPSYVSAAKAITHAIKPKSLDALNIELQNDWPCFGKFENLVVDNGAEFWSKNLEHACQSAGINIQYNPVRKPWLKPFIERFFGVMNEYFLPELPGKTFSNILEKEEYKPEKDAIMRFSTFVEEFHRWIADVYHQDSNSRETRIPIKRWQQGFDAYPPLTMNEEEETRFSMLMRISDSRTLTRNGFKYQELMYDSTALADYRKHYPQTKETVKKLIKVDPDDISKIYVYLEELESYLEVPCTDPTGYTDGLSIYEHKTIKKINREVIRESKDSLGLAKARMAIHERVKQEQEVFIESKTKAKITAVKKQAQIADVSNTGTSTIKVSEESAAPVQKHISNDNSDDWDDDLEAFE

SEQ ID NO: 60 - protein, 333 aa, Pseudoalteromonas sp.
MNALTEIQIEKLRNFSDCIVMHPQIKTIFNDFDELRLNRKFQSDQQCMLLIGDTGVGKSHTINHYKKRVLATQNYSRNTMPVLVSRISRGKGLDATLVQMLADLELFGSSQIKKRGYKTDLTKKLVESLIKAQVELLIINEFQELIEFKSVQERQQIANGLKFISEEAKVPIVLVGMPWAAKIAEEPQWASRLVRKRKLEYFSLKNDSKYFRQYLMGLAKKMPFDVPPKLESKNTTIALFAACRGENRALKHLLLEALKLALSCNEYLENKHFITAYDKFDFFNDKEKLKSKNPFKQDIKDIEIYEVIKNSSYNPNALDPEDMLTDRVFAIVK

SEQ ID NO: 61 - protein, 348 aa, synthetic construct
MNALTEIQIEKLRNFSDCIVMHPQIKTIFNDFDELRLNRKFQSDQQCMLLIGDTGVGKSHTINHYKKRVLATQNYSRNTMPVLVSRISRGKGLDATLVQMLADLELFGSSQIKKRGYKTDLTKKLVESLIKAQVELLIINEFQELIEFKSVQERQQIANGLKFISEEAKVPIVLVGMPWAAKIAEEPQWASRLVRKRKLEYFSLKNDSKYFRQYLMGLAKKMPFDVPPKLESKNTTIALFAACRGENRALKHLLLEALKLALSCNEYLENKHFITAYDKFDFFNDKEKLKSKNPFKQDIKDIEIYEVIKNSGSGPAAKKKKLDGSGSYNPNALDPEDMLTDRVFAIVK

SEQ ID NO: 62 - protein, 409 aa, Pseudoalteromonas sp.
MAFLFSPKARAFSDESLESYLLRVVSENFFDSYEGLSLAIREELHELDFEAHGAFPVDLKRLNVYHAKHNSHFRMRALGLLETLLDLPRYELQKLALLKSDIKFNSSVALYNNGVDIPLRFIRHHAEEAVDSIPVCSQCLAEEAYIKQSWHIKWVNACTKHQCALLHNCPECYAPINYIENESITHCSCGFELSCASTSPVNTLSIEHLNKLLDKGERNDSNPLFNNMTLTERFAALLWYQERYSQTDNFCLNDAVNYFSKWPAVFNTELDELSKNAEMKLIDLFNKTEFKFIFGDAILACPSTQKQSESHFIYRALLDYLVTLVESNPKTKKPNAADLLVSVLEAATLLGTSVEQVYRLYQNGILQTAFRHKMNQRINPYKGAFFLRHVIEYKTSFGNDKARMYLSAW

SEQ ID NO: 63 - protein, 452 aa, synthetic construct
MPKKKRKVGSGEQKLISEEDLEQKLISEEDLEQKLISEEDLGSGAFLFSPKARAFSDESLESYLLRVVSENFFDSYEGLSLAIREELHELDFEAHGAFPVDLKRLNVYHAKHNSHFRMRALGLLETLLDLPRYELQKLALLKSDIKFNSSVALYNNGVDIPLRFIRHHAEEAVDSIPVCSQCLAEEAYIKQSWHIKWVNACTKHQCALLHNCPECYAPINYIENESITHCSCGFELSCASTSPVNTLSIEHLNKLLDKGERNDSNPLFNNMTLTERFAALLWYQERYSQTDNFCLNDAVNYFSKWPAVFNTELDELSKNAEMKLIDLFNKTEFKFIFGDAILACPSTQKQSESHFIYRALLDYLVTLVESNPKTKKPNAADLLVSVLEAATLLGTSVEQVYRLYQNGILQTAFRHKMNQRINPYKGAFFLRHVIEYKTSFGNDKARMYLSAW

SEQ ID NO: 64 - protein, 207 aa, Vibrio parahaemolyticus
MFDQTKKSSHVHNICKFMSLKNDAVVRTLSILEFDFCFHLEYNPDVEKYLSQPHGYHYQFNNRKCRYTPDFLVFDRQERSSFIEIKHSSQILKPDFRARFAEKQRVAREEHDKRLILITEKQIRINPIFNNLKLLHRYSGLHSVTKVQKTVLGYIQRKQRVKLYEVSEYLGLSEHETLTSALCWLSSGKVKTDFKSADFSLNSYVWC

SEQ ID NO: 65 - protein, 626 aa, Vibrio parahaemolyticus
MVASELDNFVGFFDEMEASRSEAQMESQIPVELFQSDTDHSSSFDSLPEKTQKEVLRRLKIIQYVEVRLKGGWTEKNLDPILNMVENALELPRPSWRTLASWKKDYYESGKKWLSLIPKHTQKGNRTAHTDSQFIIDEAIAKKYLTRERLSVAETYRYYKSRVIKTNQTIVEGKIELISQRAFYDRVNGLPAYDVAVARYGKRYADRHFRSVGQQVSATKPMEYVEIDHTPIPVILIDDELDVPLGRPYLTMLYDRFSKCIVGLSVNFREPSFDSVRKALLNALLNKNWVKDKYPSVKNDWPCCGKIDYLVVDNGAEFWSKSLEDSLKPLVLDIQYSQAAKPWRKSGIEKLFDQLNKGLTNSLPGKTFTNPTQLEDYDPKKESVVRVSVFLELLHKWVIDYYHMSPDARERDVPYHKWHESRWLPNTYEDEEKSRLKIELGLLRHRTIGLAGIRLHNLRYQSDELIEYRKYCSVKYERKLFVKTKTDPSDISSIYVYLEFENRYIRVPAVDNSGYTQGLSLFEHERIQRVRRLNTKRMVDEEALADTYLYMESRIEAETERLRNYGDRKRSQPKIGNTSKLAKFRDVGTTGPSSIITTSVNEPLTNSYDGIVTDLDDEDFDEIEGY

SEQ ID NO: 66 - protein, 875 aa, synthetic construct
MFDQTKKSSHVHNICKFMSLKNDAVVRTLSILEFDFCFHLEYNPDVEKYLSQPHGYHYQFNNRKCRYTPDFLVFDRQERSSFIEIKHSSQILKPDFRARFAEKQRVAREEHDKRLILITEKQIRINPIFNNLKLLHRYSGLHSVTKVQKTVLGYIQRKQRVKLYEVSEYLGLSEHETLTSALCWLSSGKVKTDFKSADFSLNSYVWCGSGPKKKRKVGSGYPYDVPDYAYPYDVPDYAYPYDVPDYAGSGVASELDNFVGFFDEMEASRSEAQMESQIPVELFQSDTDHSSSFDSLPEKTQKEVLRRLKIIQYVEVRLKGGWTEKNLDPILNMVENALELPRPSWRTLASWKKDYYESGKKWLSLIPKHTQKGNRTAHTDSQFIIDEAIAKKYLTRERLSVAETYRYYKSRVIKTNQTIVEGKIELISQRAFYDRVNGLPAYDVAVARYGKRYADRHFRSVGQQVSATKPMEYVEIDHTPIPVILIDDELDVPLGRPYLTMLYDRFSKCIVGLSVNFREPSFDSVRKALLNALLNKNWVKDKYPSVKNDWPCCGKIDYLVVDNGAEFWSKSLEDSLKPLVLDIQYSQAAKPWRKSGIEKLFDQLNKGLTNSLPGKTFTNPTQLEDYDPKKESVVRVSVFLELLHKWVIDYYHMSPDARERDVPYHKWHESRWLPNTYEDEEKSRLKIELGLLRHRTIGLAGIRLHNLRYQSDELIEYRKYCSVKYERKLFVKTKTDPSDISSIYVYLEFENRYIRVPAVDNSGYTQGLSLFEHERIQRVRRLNTKRMVDEEALADTYLYMESRIEAETERLRNYGDRKRSQPKIGNTSKLAKFRDVGTTGPSSIITTSVNEPLTNSYDGIVTDLDDEDFDEIEGY

SEQ ID NO: 67 - protein, 355 aa, Vibrio parahaemolyticus
MNITPEQRAQLAAYENCFIEYPEITEIYSIFDQLRFNQSLGGEPESFLLTGEAGSGKTALIDNYLSRFEVSANSWSQQTILSTRIPSRVNEQNTLTQFLIDLDVKSGGRGVRRRNEIALAEAVVAQLKRKSVELIIVNEVQELIEFSTAQERQVIANTFKYISEEARVSFVLVGMPYASVLAQEPQWDSRLSWRRNLDYFKLFKSKINEKNTARSYEIDTLQKKHFAKFVAGLASRMGYDNPPKLTKNDTLYPLFVMCRGECRRLKHFLSDAMIMSFKESTDTIDKETLSRAFAFKFPHMANPFACSLSEIKLSQIDTNSMYNTTAIATEDRILAPRFTDDFPLSMLLSKSGVKI

SEQ ID NO: 68 - protein, 370 aa, synthetic construct
MNITPEQRAQLAAYENCFIEYPEITEIYSIFDQLRFNQSLGGEPESFLLTGEAGSGKTALIDNYLSRFEVSANSWSQQTILSTRIPSRVNEQNTLTQFLIDLDVKSGGRGVRRRNEIALAEAVVAQLKRKSVELIIVNEVQELIEFSTAQERQVIANTFKYISEEARVSFVLVGMPYASVLAQEPQWDSRLSWRRNLDYFKLFKSKINEKNTARSYEIDTLQKKHFAKFVAGLASRMGYDNPPKLTKNDTLYPLFVMCRGECRRLKHFLSDAMIMSFKESTDTIDKETLSRAFAFKFPHMANPFACSLSEIKLSQIDTNSGSGPAAKKKKLDGSGMYNTTAIATEDRILAPRFTDDFPLSMLLSKSGVKI

SEQ ID NO: 69 - protein, 403 aa, Vibrio parahaemolyticus
MNSNIQLYRDESLESFLLRLSQEQGYGRFSHFAEELWYQTLDDSSGLSGAFPLELSRVNVYHAQTTSQMRVRVFIYLENQLKLSNFRVLRLALTHSKSHFSPDLKAVHRLGVDYPYAFLRKRFTPVCPSCLSEAPYIRQHWHLIPHQVCEKHGCDLIHRCPECDALLEYQSVESITQCECGFHLLEALPKPASESDLLVARWLTGNHLEVVGPMGKAMSISERYGLLLWYVNRYGSLEEFSLGEFVQYCAMWPKRLHQDLDMLAKKAELVRIKKWKQTFFYEAFGTLLKECRYLPSRQLSKNIVLAELLRYFNRLVADHPSSVKGNIVDILLSPLEASTLLSCTTDEIYRLYEYGEIKAAVRPQMHVKIASHESVFTLRSVVETKLARMCSESDGLSVYLPEW

SEQ ID NO: 70 - protein, 446 aa, synthetic construct
MPKKKRKVGSGEQKLISEEDLEQKLISEEDLEQKLISEEDLGSGNSNIQLYRDESLESFLLRLSQEQGYGRFSHFAEELWYQTLDDSSGLSGAFPLELSRVNVYHAQTTSQMRVRVFIYLENQLKLSNFRVLRLALTHSKSHFSPDLKAVHRLGVDYPYAFLRKRFTPVCPSCLSEAPYIRQHWHLIPHQVCEKHGCDLIHRCPECDALLEYQSVESITQCECGFHLLEALPKPASESDLLVARWLTGNHLEVVGPMGKAMSISERYGLLLWYVNRYGSLEEFSLGEFVQYCAMWPKRLHQDLDMLAKKAELVRIKKWKQTFFYEAFGTLLKECRYLPSRQLSKNIVLAELLRYFNRLVADHPSSVKGNIVDILLSPLEASTLLSCTTDEIYRLYEYGEIKAAVRPQMHVKIASHESVFTLRSVVETKLARMCSESDGLSVYLPEW

SEQ ID NO: 71 - protein, 208 aa, Aliiglaciecola sp.
MKKRKLTKSAVNNIHRFASFKMDDFIEVESTLEFDACFHFEYSAKVLEFESQPIGFEYELDGKIRSYTPDYLARLETLPSTFYEVKLYKKTLSEIFKSEFKAKQVAAEALGGRLELITENNIRVYPLLDNLKILHRYHSAENDLSDQQYQAITILGRVERLSILDLIHRMGQNYREIFPDILSLVALDLLKLDMNMPISTDSIIWCSK

SEQ ID NO: 72 - protein, 616 aa, Aliiglaciecola sp.
MASEDTFSGLFDLVVEENCSMPDGLQPTEPATFRALSVFTTIQRDQAIHRLNLIKYLLKAGVRSFTEKTITPLLPDLVTEFGNDVPSWRTLARWWSLFKASDFDIVALVPQITKGNSNFKADPLLEPLIAEAIGRIMSAERPNLAEGHRFLETLVLRYNKGNDTQLQCISSEALRLRVGKITPFEEIKARKGLTAANNEFRAIGQKIKTTRILERVEVDHTRLDLFVIDDIYFIPMGRPWLTMLIDSFSLSVVGFYLGFEPPSFVSVSHALKNAILPKSYVKENYPQVNNEWICSGLIELLVTDNGREFDDKDFKVACAELGMHVGKNPTKKPYLKASVERFFGTVNSRLLASPPGKTFPNIFERDDYDPEKNAVISLSKINLLIHKWIIDDYQQDPNARWTNMPNLSWSVAAQSFPPATYNGSIDELDFKLGRRFEPKLRKEGITKDKLRYHSDRLASYRGRYGDHRVIAKQDPNNLGRIVVLDNDKKEYFFVPAVDFDYANGLTLWQHNLHRKYTKEFIKANYNHQDVVQARSEIIDIVEGCMAEMATGKRKKISVTNRVRAGRYLEADRRRELPSPNTSETVERNEKKEIPFSEESWDEDVDISEWTSSQVRK

SEQ ID NO: 73 - protein, 866 aa, synthetic construct
MKKRKLTKSAVNNIHRFASFKMDDFIEVESTLEFDACFHFEYSAKVLEFESQPIGFEYELDGKIRSYTPDYLARLETLPSTFYEVKLYKKTLSEIFKSEFKAKQVAAEALGGRLELITENNIRVYPLLDNLKILHRYHSAENDLSDQQYQAITILGRVERLSILDLIHRMGQNYREIFPDILSLVALDLLKLDMNMPISTDSIIWCSKGSGPKKKRKVGSGYPYDVPDYAYPYDVPDYAYPYDVPDYAGSGASEDTFSGLFDLVVEENCSMPDGLQPTEPATFRALSVFTTIQRDQAIHRLNLIKYLLKAGVRSFTEKTITPLLPDLVTEFGNDVPSWRTLARWWSLFKASDFDIVALVPQITKGNSNFKADPLLEPLIAEAIGRIMSAERPNLAEGHRFLETLVLRYNKGNDTQLQCISSEALRLRVGKITPFEEIKARKGLTAANNEFRAIGQKIKTTRILERVEVDHTRLDLFVIDDIYFIPMGRPWLTMLIDSFSLSVVGFYLGFEPPSFVSVSHALKNAILPKSYVKENYPQVNNEWICSGLIELLVTDNGREFDDKDFKVACAELGMHVGKNPTKKPYLKASVERFFGTVNSRLLASPPGKTFPNIFERDDYDPEKNAVISLSKINLLIHKWIIDDYQQDPNARWTNMPNLSWSVAAQSFPPATYNGSIDELDFKLGRRFEPKLRKEGITKDKLRYHSDRLASYRGRYGDHRVIAKQDPNNLGRIVVLDNDKKEYFFVPAVDFDYANGLTLWQHNLHRKYTKEFIKANYNHQDVVQARSEIIDIVEGCMAEMATGKRKKISVTNRVRAGRYLEADRRRELPSPNTSETVERNEKKEIPFSEESWDEDVDISEWTSSQVRK

SEQ ID NO: 74 - protein, 335 aa, Aliiglaciecola sp.
MSEFGEKLKLVRELFIAGPYLESLMCEIDECKEDSKLGGEAQCMFITGNTGSGKTTLIRKYMENYPRKELADRTKIPVFFTSLPENATPVRASQKMLTDLGDPFSCVSSDLEELRIKLICLLVSCGVELIIIDEFQHLIERKNNKVLHRAADWLKTIIIDSNIPVVLVGMPYSSVILDVNSQLNDRMLFKRRLPPFRVEEESERKVYLQFLKVFDLALPFPDSSSLQTREVALRLYSHSKGNLRKLRELLNQASRDALLMSANCITSEHFKSAIDKINGNYSDTVNPFNVSHINDVAIDEPDLDIGWEDFKNKPGEILVGKSSRQFTVGDIFATR

SEQ ID NO: 75 - protein, 350 aa, synthetic construct
MSEFGEKLKLVRELFIAGPYLESLMCEIDECKEDSKLGGEAQCMFITGNTGSGKTTLIRKYMENYPRKELADRTKIPVFFTSLPENATPVRASQKMLTDLGDPFSCVSSDLEELRIKLICLLVSCGVELIIIDEFQHLIERKNNKVLHRAADWLKTIIIDSNIPVVLVGMPYSSVILDVNSQLNDRMLFKRRLPPFRVEEESERKVYLQFLKVFDLALPFPDSSSLQTREVALRLYSHSKGNLRKLRELLNQASRDALLMSANCITSEHFKSAIDKINGNYSDTVNPFNVSHINDVAIDEPDLDIGSGPAAKKKKLDGSGGWEDFKNKPGEILVGKSSRQFTVGDIFATR

SEQ ID NO: 76 - protein, 398 aa, Aliiglaciecola sp.
MSMLLIRTKPFLDESLESYLLRLSIHNGYNKFQSFWAGVRSHLNESTRGIDSALPSELSKINICHANVSSAKRLDALRLVSQLTNHEPLPLLSLALFRGGQLFSRKRTSVFNNGVTIPFRFLRTKGIPICPACIKENVYIRQHWHFSLFEACPEHSVLLRNHCDCGEEINYLSSHEIAQCAKCGSNLADLEATVSSAPQREIAHWLSGRLVEGLPAVIQSHSWGICLWWQETFNDGKDIDSEQLHLFLAQWPDSLRSYLNCKLAHSKEYALKPFNQLSFKDVFGLLLIQASRLPSTNLSENIVLKEIVRYLEEHVFEPECLLSDLKLNSIEAAIILGTSVEQIAVLVDQGELQTKSRMKANSVLNANWRVLSLGDVFCLWLAKFQTDNSHSNVFISRW

SEQ ID NO: 77 - protein, 441 aa, synthetic construct
MPKKKRKVGSGEQKLISEEDLEQKLISEEDLEQKLISEEDLGSGSMLLIRTKPFLDESLESYLLRLSIHNGYNKFQSFWAGVRSHLNESTRGIDSALPSELSKINICHANVSSAKRLDALRLVSQLTNHEPLPLLSLALFRGGQLFSRKRTSVFNNGVTIPFRFLRTKGIPICPACIKENVYIRQHWHFSLFEACPEHSVLLRNHCDCGEEINYLSSHEIAQCAKCGSNLADLEATVSSAPQREIAHWLSGRLVEGLPAVIQSHSWGICLWWQETFNDGKDIDSEQLHLFLAQWPDSLRSYLNCKLAHSKEYALKPFNQLSFKDVFGLLLIQASRLPSTNLSENIVLKEIVRYLEEHVFEPECLLSDLKLNSIEAAIILGTSVEQIAVLVDQGELQTKSRMKANSVLNANWRVLSLGDVFCLWLAKFQTDNSHSNVFISRW

SEQ ID NO: 78 - protein, 210 aa, Oceanospirillum linum
MYNRNLRKPSPVKNVYKFASRKNHSTIMCESSLEFDACFHLEYSDKVVNFASQPTGIEYFDNANKKRRYTPDFSVSYQDGTSNLIEVKPAKKLLSPDFQNDFSQKLNAYKEIGETLILVTENQIRSEPTLTNYKILHRYASFLGDSELQAEIKKRLHETKNLSVARLASLLNLEEQNLIPVCAMMLAKGYLTADLQASKFTELTLTPFED

SEQ ID NO: 79 - protein, 601 aa, Oceanospirillum linum
MFEDEYSPEYIDNLDGGFIEHNEGEEDTYDLDCFPKEQQQIAVAKTKFILNIRKKLKDKGWTKENVMPIVDALYDASLPFKSPSLSSVQRWHRSLSQNQDNPAVLVSKHHRKGNRNSKVGDDKYFDLALERFLKATRPTAMSAYRYYESQMLIDIENGKYEGRPISQTAFYKRLAKLSSYEVTAKRYGKYKADMKFGYKGGPLKLERPLQRVEIDHTPLDLILLDDETLHPLGRPYLTILKDSLSKCIIGYHLSFQAPSYASASKAICHAMLPKKIKGPDGKPSWECHGKIETLVADNGAEFWSESLEHFCLEAGINIQYNKVGQPWGKGLVERNFLTIQQLILDDLEGKTFSNNVERADYNSVKNAKFKFSRFVKAFETWVAEVFNWEPNQKKTHVPMLEWRKAVNKFPPNELTPPEHEHIKLISGILKKPALQNNGIIFEHLRYDSKELADYRKQFCRDKKIKVTTKVNIDDLGFAYVYLFEYERYLKVPCVDFQYASGLSYEKHKVHITYIRKYNKIHGKSGLDQARAKQHIAEILEDIDASAKESSSKQKKVGGMKKAARVKGVDSVSVQTRREKDSNPVKQPSSLADLEMIWQEDT

SEQ ID NO: 80 - protein, 853 aa, synthetic construct
MYNRNLRKPSPVKNVYKFASRKNHSTIMCESSLEFDACFHLEYSDKVVNFASQPTGIEYFDNANKKRRYTPDFSVSYQDGTSNLIEVKPAKKLLSPDFQNDFSQKLNAYKEIGETLILVTENQIRSEPTLTNYKILHRYASFLGDSELQAEIKKRLHETKNLSVARLASLLNLEEQNLIPVCAMMLAKGYLTADLQASKFTELTLTPFEDGSGPKKKRKVGSGYPYDVPDYAYPYDVPDYAYPYDVPDYAGSGFEDEYSPEYIDNLDGGFIEHNEGEEDTYDLDCFPKEQQQIAVAKTKFILNIRKKLKDKGWTKENVMPIVDALYDASLPFKSPSLSSVQRWHRSLSQNQDNPAVLVSKHHRKGNRNSKVGDDKYFDLALERFLKATRPTAMSAYRYYESQMLIDIENGKYEGRPISQTAFYKRLAKLSSYEVTAKRYGKYKADMKFGYKGGPLKLERPLQRVEIDHTPLDLILLDDETLHPLGRPYLTILKDSLSKCIIGYHLSFQAPSYASASKAICHAMLPKKIKGPDGKPSWECHGKIETLVADNGAEFWSESLEHFCLEAGINIQYNKVGQPWGKGLVERNFLTIQQLILDDLEGKTFSNNVERADYNSVKNAKFKFSRFVKAFETWVAEVFNWEPNQKKTHVPMLEWRKAVNKFPPNELTPPEHEHIKLISGILKKPALQNNGIIFEHLRYDSKELADYRKQFCRDKKIKVTTKVNIDDLGFAYVYLFEYERYLKVPCVDFQYASGLSYEKHKVHITYIRKYNKIHGKSGLDQARAKQHIAEILEDIDASAKESSSKQKKVGGMKKAARVKGVDSVSVQTRREKDSNPVKQPSSLADLEMIWQEDT

SEQ ID NO: 81 - protein, 334 aa, Oceanospirillum linum
MPKLTDAQKANIRQFKDSFCLYYSIKKLLSDLETVFESSEIGGEPLSMLITGDTGSGKSSTINHFIKSKISPQTGRAPILSTRVPSRATAEETTKQMLIDLGVFGSSVSSRKSSDQNLTNRLISAVKDSGIKLIIINEFQELVEFKKPKDQQVISNRLKVISESTEVPLIFVGMPWSDEIRQDPQWSSRLATRSHNIEYFSIIKKPRQFRDFMKALKSHIPIQRSDDMDNMEEDLRIFAATCGEQRQIKALMTEVYRLCLIQEQPISLKIYDEAFRNLYPTANDQPFKGKLEQVNFREIEMSSRYIRGDSMYPAHIEPAKLSEFYSLSELLSKS

SEQ ID NO: 82 - protein, 349 aa, synthetic construct
MPKLTDAQKANIRQFKDSFCLYYSIKKLLSDLETVFESSEIGGEPLSMLITGDTGSGKSSTINHFIKSKISPQTGRAPILSTRVPSRATAEETTKQMLIDLGVFGSSVSSRKSSDQNLTNRLISAVKDSGIKLIIINEFQELVEFKKPKDQQVISNRLKVISESTEVPLIFVGMPWSDEIRQDPQWSSRLATRSHNIEYFSIIKKPRQFRDFMKALKSHIPIQRSDDMDNMEEDLRIFAATCGEQRQIKALMTEVYRLCLIQEQPISLKIYDEAFRNLYPTANDQPFKGKLEQVNFREIEMSSGSGPAAKKKKLDGSGRYIRGDSMYPAHIEPAKLSEFYSLSELLSKS

SEQ ID NO: 83 - protein, 386 aa, Oceanospirillum linum
MPRLPAHIQIYSDESLESYLLRLCQANYFDSFYDFALELKHLLWEQESGAAGGLPTELAAINIYHAQQDSGRRSQALFLVEKMLELKPFTLLDITFKHGTSVDLYQRATVSYQNHIIPRHYLRQNSIPICPVCLQGEQPYIRYLWHLEPVKACVEHNCKLVECCPRCNETLNYMESELITHCFCGFDLRKCEQEPADAKSYWQLNPEAFSAFGDCSFSEKLAVLSLLEQLASDKNQEVLLREGIDFFSRLLEERISEQLTLATKPLSKLSFRTLSAGLIDELSKVSNLPQGLISGVIKAILIKALDTPKTSLGCLGDSLLSPRECAFLLQSSVNDIYRLYETGVLSPAIRLPSKQTIQSYQTIFRLQDIAGFTLSCSSFMAVTSSR

SEQ ID NO: 84 - protein, 429 aa, synthetic construct
MPKKKRKVGSGEQKLISEEDLEQKLISEEDLEQKLISEEDLGSGPRLPAHIQIYSDESLESYLLRLCQANYFDSFYDFALELKHLLWEQESGAAGGLPTELAAINIYHAQQDSGRRSQALFLVEKMLELKPFTLLDITFKHGTSVDLYQRATVSYQNHIIPRHYLRQNSIPICPVCLQGEQPYIRYLWHLEPVKACVEHNCKLVECCPRCNETLNYMESELITHCFCGFDLRKCEQEPADAKSYWQLNPEAFSAFGDCSFSEKLAVLSLLEQLASDKNQEVLLREGIDFFSRLLEERISEQLTLATKPLSKLSFRTLSAGLIDELSKVSNLPQGLISGVIKAILIKALDTPKTSLGCLGDSLLSPRECAFLLQSSVNDIYRLYETGVLSPAIRLPSKQTIQSYQTIFRLQDIAGFTLSCSSFMAVTSSR

SEQ ID NO: 85 - protein, 222 aa, Vibrio anguillarum
MYHTFESLLQVWLFDMKKRILKNSKVKNISRFVSLKTDSVQTTESDLEFDACFHFEFAPQIKTFETQPLGFKYRMNGRLRRYTPDMLCYFHDGYAPYYEVKPKWVTEQDEFKEKFDAQRQQAIANGHDLLVLTEEDIQIYPLLDNLKIIHRYACSDNLDDVQIRLLKLFQNYGEMRISQVLKASQGQSASILPALYDLIAKKILEFDWHCPISHDSLIWRVS

SEQ ID NO: 86 - protein, 624 aa, Vibrio anguillarum
MSDNSEDVHAFGGFFSEKSSVISVPKTSKGAPFGTELQERYQDLFSFDEKRRDEAIHRYNILDYLIELHGPSLTLKKILGSMKGLEDKFYPNVPSPPSIYRYWNTYKKSGFVLSYLVPGVTSGNRAPRKALELEEYIDNAIKSYFSEESPTIQQAFTLLEVELDRHNECNDTQLTFEYESFRKRIVKKPDYERLLIKKGKKAADTFYKKVGHRPETTRVLQRVEADHTRLDLFVIDDARTLPLGRPWLTLLFDTHTKSVVGFYLGFEPPSYLSVSLALENAILPKDYVKELYPDVKNEWPCYGLPEHLIVDNGAEFNSKDFVSACKNLRIKVKKNPVKKPWLKGSVERYFRTINNKLLSGIPGKSFSNIFARGDYNPQKNAIITRSDLMKVIHVWLIDIYQSSPNGLETNIPNLTWADAMRSALPPRPFKGTIDELRFNLGKNAEISLDKNGIRFKKTLRYSSASLAQYFGKHTYDGKSIKVKIKYDPTCMGKIYVLDEDKHEFFAVESVDPDYAYSVSEWLHKVCCDYARDHIRNNYRHHDVIKAWRVIYDIIYEALHLSGNDKQTNIGIREASKFERVREHSERTKSQKRPELSYIDEDDIDWGIDVDTDGWKIDSVRGNQL

SEQ ID NO: 87 - protein, 888 aa, synthetic construct
MYHTFESLLQVWLFDMKKRILKNSKVKNISRFVSLKTDSVQTTESDLEFDACFHFEFAPQIKTFETQPLGFKYRMNGRLRRYTPDMLCYFHDGYAPYYEVKPKWVTEQDEFKEKFDAQRQQAIANGHDLLVLTEEDIQIYPLLDNLKIIHRYACSDNLDDVQIRLLKLFQNYGEMRISQVLKASQGQSASILPALYDLIAKKILEFDWHCPISHDSLIWRVSGSGPKKKRKVGSGYPYDVPDYAYPYDVPDYAYPYDVPDYAGSGSDNSEDVHAFGGFFSEKSSVISVPKTSKGAPFGTELQERYQDLFSFDEKRRDEAIHRYNILDYLIELHGPSLTLKKILGSMKGLEDKFYPNVPSPPSIYRYWNTYKKSGFVLSYLVPGVTSGNRAPRKALELEEYIDNAIKSYFSEESPTIQQAFTLLEVELDRHNECNDTQLTFEYESFRKRIVKKPDYERLLIKKGKKAADTFYKKVGHRPETTRVLQRVEADHTRLDLFVIDDARTLPLGRPWLTLLFDTHTKSVVGFYLGFEPPSYLSVSLALENAILPKDYVKELYPDVKNEWPCYGLPEHLIVDNGAEFNSKDFVSACKNLRIKVKKNPVKKPWLKGSVERYFRTINNKLLSGIPGKSFSNIFARGDYNPQKNAIITRSDLMKVIHVWLIDIYQSSPNGLETNIPNLTWADAMRSALPPRPFKGTIDELRFNLGKNAEISLDKNGIRFKKTLRYSSASLAQYFGKHTYDGKSIKVKIKYDPTCMGKIYVLDEDKHEFFAVESVDPDYAYSVSEWLHKVCCDYARDHIRNNYRHHDVIKAWRVIYDIIYEALHLSGNDKQTNIGIREASKFERVREHSERTKSQKRPELSYIDEDDIDWGIDVDTDGWKIDSVRGNQL

SEQ ID NO: 88 - protein, 317 aa, Vibrio anguillarum
MTASVKMLHQQVKNIFISDAQIDEILADIDECREDSDRISEPECLIVVGDSGSGKTTIIDKYLSDNPRMEANDGSIIPILFTSLPANANPVTASERLLSSMGDPLAFNHGKDPAELMKIVKDLLRECRVELIIIDEFQHMIDRKSKDVLHITADWLKMIIIESKIPVVLFGMPYSTEILRANNQLRGRFESQHHLKPFKVKKTSERIRYKTFLTMLDAALPFSTKSGLASEDLMKRVYVFSKGNMRLIRRLINKAAKFALLENAPCISLMHFARAAPKVSRDACESFNPFDVDIKQLKIIEPSDDVGWENYLAAKGD

SEQ ID NO: 89 - protein, 335 aa, synthetic construct
MTASVKMLHQQVKNIFISDAQIDEILADIDECREDSDRISEPECLIVVGDSGSGKTTIIDKYLSDNPRMEANDGSIIPILFTSLPANANPVTASERLLSSMGDPLAFNHGKDPAELMKIVKDLLRECRVELIIIDEFQHMIDRKSKDVLHITADWLKMIIIESKIPVVLFGMPYSTEILRANNQLRGRFESQHHLKPFKVKKTSERIRYKTFLTMLDAALPFSTKSGLASEDLMKRVYVFSKGNMRLIRRLINKAAKFALLENAPCISLMHFARAAPKVSRDACESFNPFDVDIKQLKIIEPSDDVGSGPAAKKKKLDGSGGWENYLAAKGDGPH

SEQ ID NO: 90 - protein, 394 aa, Vibrio anguillarum
MFLQRPKPYSDESLESFFIRVANKNGYDDVHRFLEATKRFLQDIDHHGYQTFPTDITRINPCSANNSSRARTASLLKLAQLTFNEQPELLGLALNRTNLQYSPSTSAVIRGAEVLPRSLLRTNSISSCPLCLQENGYASYLWHFKGYDHCHIHNIPLINACSCGAEFDYRVCGLKGICNNCKEPITTKNQENSYEATSTVSNWLAGNGSQDLPDIPRSYRWGLIHWWMNLNDNEFDHLSFTHFFSNWPRSFHSMIDDEIEFNLEHAVVSTSELRLKDLLGRLFFHSIRLPERNLQHNIILGELLSHLEKRLWRDKGLIANLKMNALEASVMLNCSLEQIASMVEQRILKPNRRTKPNSPIETTDYLFHFGDIFCLWLAEFQTDEFNRSFYVSRW

SEQ ID NO: 91 - protein, 437 aa, synthetic construct
MPKKKRKVGSGEQKLISEEDLEQKLISEEDLEQKLISEEDLGSGFLQRPKPYSDESLESFFIRVANKNGYDDVHRFLEATKRFLQDIDHHGYQTFPTDITRINPCSANNSSRARTASLLKLAQLTFNEQPELLGLALNRTNLQYSPSTSAVIRGAEVLPRSLLRTNSISSCPLCLQENGYASYLWHFKGYDHCHIHNIPLINACSCGAEFDYRVCGLKGICNNCKEPITTKNQENSYEATSTVSNWLAGNGSQDLPDIPRSYRWGLIHWWMNLNDNEFDHLSFTHFFSNWPRSFHSMIDDEIEFNLEHAVVSTSELRLKDLLGRLFFHSIRLPERNLQHNIILGELLSHLEKRLWRDKGLIANLKMNALEASVMLNCSLEQIASMVEQRILKPNRRTKPNSPIETTDYLFHFGDIFCLWLAEFQTDEFNRSFYVSRW

SEQ ID NO: 92 - protein, 207 aa, Halomonas sp.
MYRRKLRHSRVKNLYKFASFKTATAHTVESSLEFDACYHFEYSPHVKSFIAQPMGFTYSIHGKTNPYTPDFKIINNNQKIAFIEIKPHSKTLHPEFVQKFQAKKEAACQLGFELSLVTELQIRKYPILNNYKLLHRYAGFQSHCELYDSVYSLVKRHSPIFLHEICALYDIGFRPRVIRSLVSLIASGKLKANILEKEIGDDLLLWA

SEQ ID NO: 93 - protein, 631 aa, Halomonas sp.
MLEDPFFDESLAGIGFSHTACKKSRDEIEDVAFITIDDLDEECADKALFKYKVIKLVNKRLNGGWTKKNVEPIIYELYNEGFIDKKPGWQSVARWNAKYRVDKNLLYLVDRRAIKNNFCDFNSFSKDTFFWDAIEKKYLTRVRGSVATTYQFYKDLILIHNNENPDNKFVAVGRSAFYDRVKKLPPYICDLKRYGKRYADKKYRLINSFKKSTRVMERVEIDHTALDLILLDDTLNIPIGRPFITVLIDTFSKCIVGFYLSFRGPSYNSVRCAIINACLDKEDVLKKYPDVEKDWPCQGRIETLVVDNGAEFWSKDLERFSASIGMSIEYNPVGKPWKKPLVERIFNTYNTKFVHQIPGKTFSSAKDLEGYEPQKDALLPFSEFLYLLHIWVIDIYNQQSNSRKTHIPALSWQVGYEEFPPVIYQGLEKQRFKIESFPTVYRDLRPIGIEVDHISYSNEALVEFRKNNPPPLGQTKHKLCVKRDPSDVSYVYVYLPNLEKYIKVDATSQDFSLEGVSIFQYQVMRKALTRYIDANVDHAGLALANMKLSERMDDISNLALANKKSRSRGMKSVAAFVGIDSEGETSFESVHNNLKNKKDTKLSFFEGETLDDKKLKSIDDWNEIADNLEPY

SEQ ID NO: 94 - protein, 880 aa, synthetic construct
MYRRKLRHSRVKNLYKFASFKTATAHTVESSLEFDACYHFEYSPHVKSFIAQPMGFTYSIHGKTNPYTPDFKIINNNQKIAFIEIKPHSKTLHPEFVQKFQAKKEAACQLGFELSLVTELQIRKYPILNNYKLLHRYAGFQSHCELYDSVYSLVKRHSPIFLHEICALYDIGFRPRVIRSLVSLIASGKLKANILEKEIGDDLLLWAGSGPKKKRKVGSGYPYDVPDYAYPYDVPDYAYPYDVPDYAGSGLEDPFFDESLAGIGFSHTACKKSRDEIEDVAFITIDDLDEECADKALFKYKVIKLVNKRLNGGWTKKNVEPIIYELYNEGFIDKKPGWQSVARWNAKYRVDKNLLYLVDRRAIKNNFCDFNSFSKDTFFWDAIEKKYLTRVRGSVATTYQFYKDLILIHNNENPDNKFVAVGRSAFYDRVKKLPPYICDLKRYGKRYADKKYRLINSFKKSTRVMERVEIDHTALDLILLDDTLNIPIGRPFITVLIDTFSKCIVGFYLSFRGPSYNSVRCAIINACLDKEDVLKKYPDVEKDWPCQGRIETLVVDNGAEFWSKDLERFSASIGMSIEYNPVGKPWKKPLVERIFNTYNTKFVHQIPGKTFSSAKDLEGYEPQKDALLPFSEFLYLLHIWVIDIYNQQSNSRKTHIPALSWQVGYEEFPPVIYQGLEKQRFKIESFPTVYRDLRPIGIEVDHISYSNEALVEFRKNNPPPLGQTKHKLCVKRDPSDVSYVYVYLPNLEKYIKVDATSQDFSLEGVSIFQYQVMRKALTRYIDANVDHAGLALANMKLSERMDDISNLALANKKSRSRGMKSVAAFVGIDSEGETSFESVHNNLKNKKDTKLSFFEGETLDDKKLKSIDDWNEIADNLEPY

SEQ ID NO: 95 - protein, 339 aa, Halomonas sp.
MNIIHSECNQRRLYKFLNCFVQHAAMKKTLNSLYRLKNNQILGGEQQCMLITGDTGSGKSALIKEFSSAFPSYEENGVLIQPVLVSRIPSKPDVEKMMIELMNDLGQFGSEARKGRRREIGLAEALVKMLKVCKTQIIIINEFQELIEFKSVEDRQRIANRLKLISEQAGIPVVLVGMPWASEISNEPQWASRLMCKIELPYFKFLNEDDRKEFTCFVKGLACRMGYEKPPKFEIDEILFPLFSATRGEARKVKHILSEALSLALWRGENTVHQQHLAEVMDSAFFYEDNPFKLPLNEVPLCEVSKYASYNRYSTVESDMFVSTQFTPKIPTKVLFSKS

SEQ ID NO: 96 - protein, 354 aa, synthetic construct
MNIIHSECNQRRLYKFLNCFVQHAAMKKTLNSLYRLKNNQILGGEQQCMLITGDTGSGKSALIKEFSSAFPSYEENGVLIQPVLVSRIPSKPDVEKMMIELMNDLGQFGSEARKGRRREIGLAEALVKMLKVCKTQIIIINEFQELIEFKSVEDRQRIANRLKLISEQAGIPVVLVGMPWASEISNEPQWASRLMCKIELPYFKFLNEDDRKEFTCFVKGLACRMGYEKPPKFEIDEILFPLFSATRGEARKVKHILSEALSLALWRGENTVHQQHLAEVMDSAFFYEDNPFKLPLNEVPLCEVSKYAGSGPAAKKKKLDGSGSYNRYSTVESDMFVSTQFTPKIPTKVLFSKS

SEQ ID NO: 97 - protein, 412 aa, Halomonas sp.
MKLLVRPRPFINESLESYMLRLSQENFFEYYQQLSRAIKDWLQLHDHEAAGAFPEELSRLNVYHAAQSSSRRIRALKLVESLTDNEKLPLLHLAVMHSSEKFCSRYSSVFYAGSHVPRALVRQKGIPVCPDCLTEANYIRQEWHWMPYEACINHGKQMLHECPKCEEKLNYTHSECLHTCRCGFDLRNADTEPADEWQLIASRLVVGEPSPSNHPLLDIRSVSLRLACLLWYQLYAYKTLDASDQVPTLTIERAIEYFTHWPEVFTQELEQQAALSGDKLVCDYNKTSLRDVFGNIVGISRLLLKAYPESDFVLTPLENFLVRLVDQNPQSRVPNVADLLISMPEAAILLGTSYEQAYRLYEEGYLKCAVKFKSHEKLVNGIGVFYLREIMELRQSRMPVETSSYNNYLPAW

SEQ ID NO: 98 - protein, 455 aa, synthetic construct
MPKKKRKVGSGEQKLISEEDLEQKLISEEDLEQKLISEEDLGSGKLLVRPRPFINESLESYMLRLSQENFFEYYQQLSRAIKDWLQLHDHEAAGAFPEELSRLNVYHAAQSSSRRIRALKLVESLTDNEKLPLLHLAVMHSSEKFCSRYSSVFYAGSHVPRALVRQKGIPVCPDCLTEANYIRQEWHWMPYEACINHGKQMLHECPKCEEKLNYTHSECLHTCRCGFDLRNADTEPADEWQLIASRLVVGEPSPSNHPLLDIRSVSLRLACLLWYQLYAYKTLDASDQVPTLTIERAIEYFTHWPEVFTQELEQQAALSGDKLVCDYNKTSLRDVFGNIVGISRLLLKAYPESDFVLTPLENFLVRLVDQNPQSRVPNVADLLISMPEAAILLGTSYEQAYRLYEEGYLKCAVKFKSHEKLVNGIGVFYLREIMELRQSRMPVETSSYNNYLPAW

SEQ ID NO: 99 - protein, 211 aa, Vibrio sp.
MYPHTIDKPHAKKNIFKFISVKNKAIIMCESSLEFDACFHLEYHPDVASFESQPFYLEYQLEDGSHSYTPDFLVTLNDGKKYLQEVKNSKLCLTPEYLHVFEAMQRGSEDIGFPLYLVTERQIRKAFILDNLKLIHRYAGSKHICSFKQSLLEVIQNQGLSSSEKLAGIFGKSIGFMNRELLELMSLGLVSAHFETSMFDERTAIWVSEQV

SEQ ID NO: 100 - protein, 638 aa, Vibrio sp.
MSGPFVDESKGEPPNNGEGGLVQDVNIGDVTTDSSDLCYRPLNTLTVYERDLDSFPEELKNEALERFKLLSLIGKEFDGPWPFKQIQKLIEKYKNDVSIPTPSPRTVQRWRERYEKSNGDLKSLIVRNYAKGNRKPKIIGDEYYFDLAVQSWLEAERPNITRAYERYCDSIEVANESIVVGKIPSASYKSFSRRLKQLPPYAVALQRHGKYFADLWFRHNAKHKPPTRILERVEIDHTQLDLMLLHDEYLVPIGRPCLTMLIDVFSGCIIGFHLGFHAPGYATVAKALLNAMKPKDYVKDLPIELNNEWICEGKIEKLVMDNGAEFWSKSIDDACKELNIAVQYNPVKKPWLKPFIERSFGILNKTLLSTIPGKTFSNVLEKGDYDAANKAVMKFSTFVEELHRWIIDVHNAKPDSRNNRLPNLYWSQGVKTLPPARLPIKDSEQLSIIMGILVKRKLTEKGIQYEDLFYRSQALADYRARFPQTKESAIKTIKVDPDDLSRIFIFLEELNGYIKVPCDDPEGYTKHLSLHEHIIIKRAHKQYIKGHVDTLSLAKARLALAARMEEETEELRSFKRKRKPPKNIKKMAEYSGLSSAAIESKSPALDKMSSRKSNAEEPKDIANFLDDWEAILGDLSDD

SEQ ID NO: 101 - protein, 891 aa, synthetic construct
MYPHTIDKPHAKKNIFKFISVKNKAIIMCESSLEFDACFHLEYHPDVASFESQPFYLEYQLEDGSHSYTPDFLVTLNDGKKYLQEVKNSKLCLTPEYLHVFEAMQRGSEDIGFPLYLVTERQIRKAFILDNLKLIHRYAGSKHICSFKQSLLEVIQNQGLSSSEKLAGIFGKSIGFMNRELLELMSLGLVSAHFETSMFDERTAIWVSEQVGSGPKKKRKVGSGYPYDVPDYAYPYDVPDYAYPYDVPDYAGSGSGPFVDESKGEPPNNGEGGLVQDVNIGDVTTDSSDLCYRPLNTLTVYERDLDSFPEELKNEALERFKLLSLIGKEFDGPWPFKQIQKLIEKYKNDVSIPTPSPRTVQRWRERYEKSNGDLKSLIVRNYAKGNRKPKIIGDEYYFDLAVQSWLEAERPNITRAYERYCDSIEVANESIVVGKIPSASYKSFSRRLKQLPPYAVALQRHGKYFADLWFRHNAKHKPPTRILERVEIDHTQLDLMLLHDEYLVPIGRPCLTMLIDVFSGCIIGFHLGFHAPGYATVAKALLNAMKPKDYVKDLPIELNNEWICEGKIEKLVMDNGAEFWSKSIDDACKELNIAVQYNPVKKPWLKPFIERSFGILNKTLLSTIPGKTFSNVLEKGDYDAANKAVMKFSTFVEELHRWIIDVHNAKPDSRNNRLPNLYWSQGVKTLPPARLPIKDSEQLSIIMGILVKRKLTEKGIQYEDLFYRSQALADYRARFPQTKESAIKTIKVDPDDLSRIFIFLEELNGYIKVPCDDPEGYTKHLSLHEHIIIKRAHKQYIKGHVDTLSLAKARLALAARMEEETEELRSFKRKRKPPKNIKKMAEYSGLSSAAIESKSPALDKMSSRKSNAEEPKDIANFLDDWEAILGDLSDD

SEQ ID NO: 102 - protein, 339 aa, Vibrio sp.
MTSNSENVQRLVSNFNQSFALFPPFDAILSDLEKLRNKSAREGFKPSMLIYGDTGAGKSALLEHFTKESKSKTGRKVLRTRVRPSLQETLSWTLHVLNPLRRNNRFVKNASEIGLTDMLIRELKQANIGIMIIDECQEFVEIRSNDDKKEISIRLKMISEEASVSMIFVGMPWSKEITRDSQWESRIRLVREIPYFKVINENGSNNKKEMKRFALSLMEISKLMPLDKQPQLELPEFSFPLLAYSRGEMRALKDILSDALEIALNEGAKELTRYHLQEAAKFSVEGENPFDEKVNMIQIQTIQQYTRFELDDKTGRRERFDRAFTALQQIPINKLLSKR

SEQ ID NO: 103 - protein, 354 aa, synthetic construct
MTSNSENVQRLVSNFNQSFALFPPFDAILSDLEKLRNKSAREGFKPSMLIYGDTGAGKSALLEHFTKESKSKTGRKVLRTRVRPSLQETLSWTLHVLNPLRRNNRFVKNASEIGLTDMLIRELKQANIGIMIIDECQEFVEIRSNDDKKEISIRLKMISEEASVSMIFVGMPWSKEITRDSQWESRIRLVREIPYFKVINENGSNNKKEMKRFALSLMEISKLMPLDKQPQLELPEFSFPLLAYSRGEMRALKDILSDALEIALNEGAKELTRYHLQEAAKFSVEGENPFDEKVNMIQIQTIQQYTGSGPAAKKKKLDGSGRFELDDKTGRRERFDRAFTALQQIPINKLLSKR

SEQ ID NO: 104 - protein, 403 aa, Vibrio sp.
VITNIQLYPDESLESFLLRLSQEQGYERFSHFAEDIWYQTLNENEAMSGAFPLELNCINIYHGHTTSEMRARVLIDLERRIKLNDFGVLRLALMHSKANFSPKFKAVHRFGMDYPFSFLRKRFTPICPMCLGDAPYIRQNWQFIPVQSCAEHGCKLLHQCPECGCRLEYQNSERIQYCECGSNLAEAEAKVSFESELMVARWLAGKSPMEEGVMSKDMTTSERYGFLLWYVNRYGDLEDISFHAFVKYCAEWPKPLHHELDKLVDKADVIRVKQWRKVFFREVFGELLKECRELPSRQLSKNIVLVEILHYLTRLVADSSSSPKGNIADVLLSPFEASTLLSCSTDEVYRLYNFGEIQAAFRPKIHTKLARHEPVFTLRGMIETKLVRMCSESDGLSVYLSNW

SEQ ID NO: 105 - protein, 446 aa, synthetic construct
MPKKKRKVGSGEQKLISEEDLEQKLISEEDLEQKLISEEDLGSGITNIQLYPDESLESFLLRLSQEQGYERFSHFAEDIWYQTLNENEAMSGAFPLELNCINIYHGHTTSEMRARVLIDLERRIKLNDFGVLRLALMHSKANFSPKFKAVHRFGMDYPFSFLRKRFTPICPMCLGDAPYIRQNWQFIPVQSCAEHGCKLLHQCPECGCRLEYQNSERIQYCECGSNLAEAEAKVSFESELMVARWLAGKSPMEEGVMSKDMTTSERYGFLLWYVNRYGDLEDISFHAFVKYCAEWPKPLHHELDKLVDKADVIRVKQWRKVFFREVFGELLKECRELPSRQLSKNIVLVEILHYLTRLVADSSSSPKGNIADVLLSPFEASTLLSCSTDEVYRLYNFGEIQAAFRPKIHTKLARHEPVFTLRGMIETKLVRMCSESDGLSVYLSNW

SEQ ID NO: 106 - protein, 208 aa, Photobacterium aquae
MYIRNLRKPSPNKNIYKFASSKNRKTVMCEGGLEKDCCYHFEYDPEVVCYESQPEGYYYEFCGKQLPYTPDFLVHYIGGYQCFVESKPYGQTLSKEFKQQFQARKSAAERLGFDLILVTDRQIRKGYYLENCKVVHRYSGCIKGDNLPDALYDQLLDTKPIKIIDLALKVELSVGVVFAAVLRLVTLGKALIDLDSAKLNETTLVMVK

SEQ ID NO: 107 - protein, 641 aa, Photobacterium aquae
MAGRFKDEFDANYSEDDEKEFLESPESKRNRLQYGSLDSAKIIERDLDSFPEEQKTKALERYKLLSLVSKELVGGWTPKNLNPLIDKYFEKTTLTQKPSYKTLIRWHNSFNQAKGSFTGLVDKHHQKGNRTARVVGDESYYEKALERFLDAVRPSIRAAYNVYCDNITVANENIVSGKIPQVSYQTFKNRIQKEQPYSVALARHGKYYADKLYNYYQSVDMPTRILERVEMDHTPLDLILLHDDLLVPLGRAHLTLLVDIFSGCIIGFHLGFKHPSYVSASKAIIHATKNKDYISGLPIEFENKWLCEGKIENLVVDNGPEFWSKSLEDSCLEAGINVVFNKVRKPWLKPFVERKFGEIIQGIVGWVPGKTFSNVLEKEDYNPEKDAVMRFSVFVEELHRWIVDVHNASADSRKARIPNLYWRKSYEVMPPLKLLPENEHTFTIAMGSLHHRKLTSKGIKFKHIDYDSTALAQYRKEYPQTKASAIKKIKVDPDDISTIYIYLEELNGYVEVPSKDSKGYTRKLSLCEHEKLVKAHRDYIDGEIDVLSLAKARLALHERIQSEQENLQHMSLSERKRKAKATKKIAELSSVNSDTPKAQLTDKLPPNPSMSGCSGSPAEKEINPIENFRSKWNKRRKERNG

SEQ ID NO: 108 - protein, 891 aa, synthetic construct
MYIRNLRKPSPNKNIYKFASSKNRKTVMCEGGLEKDCCYHFEYDPEVVCYESQPEGYYYEFCGKQLPYTPDFLVHYIGGYQCFVESKPYGQTLSKEFKQQFQARKSAAERLGFDLILVTDRQIRKGYYLENCKVVHRYSGCIKGDNLPDALYDQLLDTKPIKIIDLALKVELSVGVVFAAVLRLVTLGKALIDLDSAKLNETTLVMVKGSGPKKKRKVGSGYPYDVPDYAYPYDVPDYAYPYDVPDYAGSGAGRFKDEFDANYSEDDEKEFLESPESKRNRLQYGSLDSAKIIERDLDSFPEEQKTKALERYKLLSLVSKELVGGWTPKNLNPLIDKYFEKTTLTQKPSYKTLIRWHNSFNQAKGSFTGLVDKHHQKGNRTARVVGDESYYEKALERFLDAVRPSIRAAYNVYCDNITVANENIVSGKIPQVSYQTFKNRIQKEQPYSVALARHGKYYADKLYNYYQSVDMPTRILERVEMDHTPLDLILLHDDLLVPLGRAHLTLLVDIFSGCIIGFHLGFKHPSYVSASKAIIHATKNKDYISGLPIEFENKWLCEGKIENLVVDNGPEFWSKSLEDSCLEAGINVVFNKVRKPWLKPFVERKFGEIIQGIVGWVPGKTFSNVLEKEDYNPEKDAVMRFSVFVEELHRWIVDVHNASADSRKARIPNLYWRKSYEVMPPLKLLPENEHTFTIAMGSLHHRKLTSKGIKFKHIDYDSTALAQYRKEYPQTKASAIKKIKVDPDDISTIYIYLEELNGYVEVPSKDSKGYTRKLSLCEHEKLVKAHRDYIDGEIDVLSLAKARLALHERIQSEQENLQHMSLSERKRKAKATKKIAELSSVNSDTPKAQLTDKLPPNPSMSGCSGSPAEKEINPIENFRSKWNKRRKERNG

SEQ ID NO: 109 - protein, 327 aa, Photobacterium aquae
MPDSNLELSIDTTLATYHASFTIYPEVEKVFSGLDWLVKRRCFGSFVPSMLLTGGTGSGKSALIKHYISKCLSENEVLLTRVRPTLKETLLWIVNEIDKYKKYRAKGSVLGLIDYVIRCVKRTELKLLVIEECQELFECTSHKERQEIRDKLKMISDECRLPIVFVGIPSAKLILEDSQWDRRIMVKRELPYFKITDEASIDRYLDLLEAMERAVPLPFDVDLMDVEIAMRLLAASHGMLGMLKELIAVGLESALISNKAAIQLEDFILGYEMIFGLDEINPFSVDINELVIKQIESYEEYVPDAETGELKFVGQIFNALTIKQLLG

SEQ ID NO: 110 - protein, 342 aa, synthetic construct
MPDSNLELSIDTTLATYHASFTIYPEVEKVFSGLDWLVKRRCFGSFVPSMLLTGGTGSGKSALIKHYISKCLSENEVLLTRVRPTLKETLLWIVNEIDKYKKYRAKGSVLGLIDYVIRCVKRTELKLLVIEECQELFECTSHKERQEIRDKLKMISDECRLPIVFVGIPSAKLILEDSQWDRRIMVKRELPYFKITDEASIDRYLDLLEAMERAVPLPFDVDLMDVEIAMRLLAASHGMLGMLKELIAVGLESALISNKAAIQLEDFILGYEMIFGLDEINPFSVDINELVIKQIESYEGSGPAAKKKKLDGSGEYVPDAETGELKFVGQIFNALTIKQLLG

SEQ ID NO: 111 - protein, 403 aa, Photobacterium aquae
MNTDIQFYPDESLESFLLRLSHHQGYERFAYFAEDIWYQTRDQHEAIAGAFPLELNRVNVYHAHTTSQMRVRVLMHLENQLNLDDFRVLHIVLAHSKSQFSPDFKAVHRCGVDYPFAFLRKRFMPVCPLCLAESAYVRQHWHFIPIQACEQHGCKLIHRCPACDGLLEYQSTECMTHCECGFNLLSTPTTSASASELLISRWLTGTQLDVAGLMGKALSISERYGFLLWYVNRYGDLEDISFDVFVEYCTTWPKSLRKDLDECVQKADAIRVKRWKQVFFSEAFGALLKGCRQLPSRQLSKNCVLVEILNYFKRLVADNPKSSKGNIADVLLSPLEASTLLSCTTDEVYRLYEFGEIKAAMRPKIHTKIASHESAFTLRSVVETRLTRMCSENDGLSVYLPEW

SEQ ID NO: 112 - protein, 446 aa, synthetic construct
MPKKKRKVGSGEQKLISEEDLEQKLISEEDLEQKLISEEDLGSGNTDIQFYPDESLESFLLRLSHHQGYERFAYFAEDIWYQTRDQHEAIAGAFPLELNRVNVYHAHTTSQMRVRVLMHLENQLNLDDFRVLHIVLAHSKSQFSPDFKAVHRCGVDYPFAFLRKRFMPVCPLCLAESAYVRQHWHFIPIQACEQHGCKLIHRCPACDGLLEYQSTECMTHCECGFNLLSTPTTSASASELLISRWLTGTQLDVAGLMGKALSISERYGFLLWYVNRYGDLEDISFDVFVEYCTTWPKSLRKDLDECVQKADAIRVKRWKQVFFSEAFGALLKGCRQLPSRQLSKNCVLVEILNYFKRLVADNPKSSKGNIADVLLSPLEASTLLSCTTDEVYRLYEFGEIKAAMRPKIHTKIASHESAFTLRSVVETRLTRMCSENDGLSVYLPEW

SEQ ID NO: 113 - protein, 208 aa, Enterovibrio coralii
MYRRNLKHSRVKNLFKFCSLKNGSVLTVESALEFDTCFHLEYCKDIVCFEAQPEGFYYQFEGKKLPYTPDFRVSYEDRREVFLEIKPASKIEGDEFRRKFVGKMEVAKSLGCPLSLVTDNQIRVNPVLYNLKLLHRYTGIVGINAIQAQLLKVVRASGLVSINDLSQRVHVSSGELKANALALISRGQLQAELNKEKFGMHSVVWIGA

SEQ ID NO: 114 - protein, 626 aa, Enterovibrio coralii
MNSSDDDDSLPLFSNEFSPSSSSEYKPNSSPPKEPQKLIERDLDSYPAHLKEEAIKRFRLLAFINKNLNGGWTPKNLNPLIQQHFEETGQSDPPKSRVVCNWRKSYELSGGKITALVPKHHRKGNYELKNTGDGAIFHDALERFLNARRPSMTTAYEYYKDQILLSNERLVEGVIKPLSYSGFKKRIKQLPPYQVAVARHGKFMADQWYGYFSAHKPPTRILEKVEIDHTPLDLILIDDELFVPFGRPYLTLLIDVFSSCIVGFHLGYKAPSYDSVSKAIIHATKPKDYLDSIASDFQHDWPCCGKIETLVVDNGAEFWSESLAQACLESGINIQFNPVRKPWLKPFVERLFGTINQKFLDPFPGKTFSSVLEKEEYNPEKDAVIRFSTFIELFHRWIVDVYHHDADSRKTRIPAKLWQQGYEDYPPLAMSQEDIDKLTVVMGVKWQPTLTRLGFKIKHLRYDCPELSEYRKRYPQTESSRKKLVKIDPDDISRIFVYLEELDGYLEVPCEDPIGYTKNLSWHQHQVLAHSHHKFIEGSIDVLSLAKARLAIHQRVQQEQEEYRLLPSKVKRERGQRKLAEFSGVEQGGNSTVALPSKAAKKDSKDEGVKGLLDDWDDMISNLDGY

SEQ ID NO: 115 - protein, 876 aa, synthetic construct
MYRRNLKHSRVKNLFKFCSLKNGSVLTVESALEFDTCFHLEYCKDIVCFEAQPEGFYYQFEGKKLPYTPDFRVSYEDRREVFLEIKPASKIEGDEFRRKFVGKMEVAKSLGCPLSLVTDNQIRVNPVLYNLKLLHRYTGIVGINAIQAQLLKVVRASGLVSINDLSQRVHVSSGELKANALALISRGQLQAELNKEKFGMHSVVWIGAGSGPKKKRKVGSGYPYDVPDYAYPYDVPDYAYPYDVPDYAGSGNSSDDDDSLPLFSNEFSPSSSSEYKPNSSPPKEPQKLIERDLDSYPAHLKEEAIKRFRLLAFINKNLNGGWTPKNLNPLIQQHFEETGQSDPPKSRVVCNWRKSYELSGGKITALVPKHHRKGNYELKNTGDGAIFHDALERFLNARRPSMTTAYEYYKDQILLSNERLVEGVIKPLSYSGFKKRIKQLPPYQVAVARHGKFMADQWYGYFSAHKPPTRILEKVEIDHTPLDLILIDDELFVPFGRPYLTLLIDVFSSCIVGFHLGYKAPSYDSVSKAIIHATKPKDYLDSIASDFQHDWPCCGKIETLVVDNGAEFWSESLAQACLESGINIQFNPVRKPWLKPFVERLFGTINQKFLDPFPGKTFSSVLEKEEYNPEKDAVIRFSTFIELFHRWIVDVYHHDADSRKTRIPAKLWQQGYEDYPPLAMSQEDIDKLTVVMGVKWQPTLTRLGFKIKHLRYDCPELSEYRKRYPQTESSRKKLVKIDPDDISRIFVYLEELDGYLEVPCEDPIGYTKNLSWHQHQVLAHSHHKFIEGSIDVLSLAKARLAIHQRVQQEQEEYRLLPSKVKRERGQRKLAEFSGVEQGGNSTVALPSKAAKKDSKDEGVKGLLDDWDDMISNLDGY

SEQ ID NO: 116 - protein, 315 aa, Enterovibrio coralii
MTEIMGDFDRLRQNRLLGGDQQCMLLTGDTGCGKSHLIRYYQSREQSEPKGRFDSSPILVSRIPSKLSLEETVLQLLKDLGQFGTTTRGRSRITTDSSLTHSLVELLRKKQVELIIINEFQELIEYKSAEKKQAIANRLKYISEEAGVPIVLVGMPWAEMIAEEPQWSSRLVTRRSLPYFKLSEDPVHFVQFLKGLAKKMPFDKPPKLEDKKTSISLFAASRGELRALRHLINDAVKDAVLEDEREFNVQRLHHSFTKLNPQVRNPFELPLNEIKLSEIEHYSGYNPRAMSNDDALTNRMFSENIPLKELLKKKG

SEQ ID NO: 117 - protein, 354 aa, synthetic construct
MKAMTEEQSVKLKAFLNCFVEYPLLTEIMGDFDRLRQNRLLGGDQQCMLLTGDTGCGKSHLIRYYQSREQSEPKGRFDSSPILVSRIPSKLSLEETVLQLLKDLGQFGTTTRGRSRITTDSSLTHSLVELLRKKQVELIIINEFQELIEYKSAEKKQAIANRLKYISEEAGVPIVLVGMPWAEMIAEEPQWSSRLVTRRSLPYFKLSEDPVHFVQFLKGLAKKMPFDKPPKLEDKKTSISLFAASRGELRALRHLINDAVKDAVLEDEREFNVQRLHHSFTKLNPQVRNPFELPLNEIKLSEIEHYSGSGPAAKKKKLDGSGGYNPRAMSNDDALTNRMFSENIPLKELLKKKG

SEQ ID NO: 118 - protein, 411 aa, Enterovibrio coralii
MSKLSIRIEHRIDESLESYLLRLSQANYFESYQLLSRAVKDWLYEHDEEAFGAFPLQFKTVNVYHAAQSSGFRVRALRLIDWLADTELPLLQLALLGSSTRFCFAHASVFRQGTHIPLCFVRKAGVPICPECLKESEHIPQVWHFLPYIACHKHHLDLIETCPSCGALVDYLTSEKVSECECGFDLKNAPTHKADPLRVLLSCLAVGDPFDFDETALGQCNQSTRFGALLWYHLEFVGNLEQGNAINVEGLSGAIGFFNKWPESFHTAMDRRLATWEASRYIEYNHTPFRKIFGDVLLHSSRLPSKDLSHNFVLRELLAYLSHLVLRHPKSKTANAGDVLLTLSETASLLSTSYEQVERLYQEGFLKLIYRPHQQTTIPPHKPAFRLRNVIELGVARMQTDVSSDVYLPAW

SEQ ID NO: 119 - protein, 454 aa, synthetic construct
MPKKKRKVGSGEQKLISEEDLEQKLISEEDLEQKLISEEDLGSGSKLSIRIEHRIDESLESYLLRLSQANYFESYQLLSRAVKDWLYEHDEEAFGAFPLQFKTVNVYHAAQSSGFRVRALRLIDWLADTELPLLQLALLGSSTRFCFAHASVFRQGTHIPLCFVRKAGVPICPECLKESEHIPQVWHFLPYIACHKHHLDLIETCPSCGALVDYLTSEKVSECECGFDLKNAPTHKADPLRVLLSCLAVGDPFDFDETALGQCNQSTRFGALLWYHLEFVGNLEQGNAINVEGLSGAIGFFNKWPESFHTAMDRRLATWEASRYIEYNHTPFRKIFGDVLLHSSRLPSKDLSHNFVLRELLAYLSHLVLRHPKSKTANAGDVLLTLSETASLLSTSYEQVERLYQEGFLKLIYRPHQQTTIPPHKPAFRLRNVIELGVARMQTDVSSDVYLPAW

SEQ ID NO: 120 - protein, 229 aa, Vibrio chagasii
MSALPSLSTATLIALESAFDTPARSLTKSRGKNIHRYVSAKMGKRVTVESFLECAACYHFDFEPSIVRFCSQPIRLSYCLNGKTHTYVPDFLVQFDTGDYKLYEVKSDMESSKEEFHCEWEAKVQGAFGIGLDLELVTEEEILNEVIFSNLKLLHRYASRDHLNDFHQTLLATLKPNGTQTARSLGHHLGLSGRKILPILCDLLSRNLLQTNLETPLSLESEFELVCYD

SEQ ID NO: 121 - protein, 603 aa, Vibrio chagasii
MTKKSFSSFHRKSVLHQEKLEQNDRVVDINDVAEATYKDISAFPEKIVVEITFRLSILRLLGRKCEKIVPKSIEPHRVDLQRSHDRKIPSAITIYRWWLTFRESDYNPVSLAPDFKSRGNRDPKVAPIVDAIMKQAVESVISGRKININSAYRRVKRKVRQYNLTHSTKYKYPEYESVRIRVKKKTPFEILAAKKGERVAKREFRRMGRKILTSSVLERVEIDHTVLDLFAVHEEHRIPLGRPWLTQLVDCYSKAVIGFYLGFEPPSYMSVSLALKNAIQRKDTLLSSYPSIENEWLCYGIPDLLVTDNGKEFLSKAFDKACESLLINVHQNKVETPDNKPHVERNYGTINTSLLDDLPGKAFSQYLQREGYDSVSEATLTLDEIKEIYLIWLVDIYHRKPNQRGTNCPNVAWRQGCQNWEPEEFLGSKDELDFKFAIEDHKQLTKAGITVSKGLTYSSERLAGYMGKKGNHKVQFKYNPECMAVIWVLDEDVNEYFTVNAIDYESARRVSLWQHKYNMKYQAELNSAEYDEDKEIDAEIKIEEIADRSILETKKIRSRRRGARHQENSARAKSISNTKLVPPQKDEEEIVIVDNEDWDIDYV

SEQ ID NO: 122 - protein, 874 aa, synthetic construct
MSALPSLSTATLIALESAFDTPARSLTKSRGKNIHRYVSAKMGKRVTVESFLECAACYHFDFEPSIVRFCSQPIRLSYCLNGKTHTYVPDFLVQFDTGDYKLYEVKSDMESSKEEFHCEWEAKVQGAFGIGLDLELVTEEEILNEVIFSNLKLLHRYASRDHLNDFHQTLLATLKPNGTQTARSLGHHLGLSGRKILPILCDLLSRNLLQTNLETPLSLESEFELVCYDGSGPKKKRKVGSGYPYDVPDYAYPYDVPDYAYPYDVPDYAGSGTKKSFSSFHRKSVLHQEKLEQNDRVVDINDVAEATYKDISAFPEKIVVEITFRLSILRLLGRKCEKIVPKSIEPHRVDLQRSHDRKIPSAITIYRWWLTFRESDYNPVSLAPDFKSRGNRDPKVAPIVDAIMKQAVESVISGRKININSAYRRVKRKVRQYNLTHSTKYKYPEYESVRIRVKKKTPFEILAAKKGERVAKREFRRMGRKILTSSVLERVEIDHTVLDLFAVHEEHRIPLGRPWLTQLVDCYSKAVIGFYLGFEPPSYMSVSLALKNAIQRKDTLLSSYPSIENEWLCYGIPDLLVTDNGKEFLSKAFDKACESLLINVHQNKVETPDNKPHVERNYGTINTSLLDDLPGKAFSQYLQREGYDSVSEATLTLDEIKEIYLIWLVDIYHRKPNQRGTNCPNVAWRQGCQNWEPEEFLGSKDELDFKFAIEDHKQLTKAGITVSKGLTYSSERLAGYMGKKGNHKVQFKYNPECMAVIWVLDEDVNEYFTVNAIDYESARRVSLWQHKYNMKYQAELNSAEYDEDKEIDAEIKIEEIADRSILETKKIRSRRRGARHQENSARAKSISNTKLVPPQKDEEEIVIVDNEDWDIDYV

SEQ ID NO: 123 - protein, 329 aa, Vibrio chagasii
MDEDRETRISKAKRAFVSTPSVTKILGYMDRCRELSDFESEPTCMMVFGASGVGKTTIIKKYLSQNKRDSEARGDVVPVLHIELPDNAKPVDAARELLLEMRDPLALYETDLARLTKRLTDLIPVTGVKLIIIDEFQHLVEERSNRVLTQVGNWLKMILNRTKCPIVLFGMPYSKVVLKANSQLHGRFSIQFELRPFNYQNGEGVFKTFLEHLDKALPFEKEVGLVEQGLQKKLYAFSQGNMRSLRNLIYQASVEAIDKQHETITEQDLIFASKLTSGDKSDRWENPFEKGVKVTEGMLRSPPKDIGWEDYYHHVTSLNAKRNGGNMFE

SEQ ID NO: 124 - protein, 344 aa, synthetic construct
MDEDRETRISKAKRAFVSTPSVTKILGYMDRCRELSDFESEPTCMMVFGASGVGKTTIIKKYLSQNKRDSEARGDVVPVLHIELPDNAKPVDAARELLLEMRDPLALYETDLARLTKRLTDLIPVTGVKLIIIDEFQHLVEERSNRVLTQVGNWLKMILNRTKCPIVLFGMPYSKVVLKANSQLHGRFSIQFELRPFNYQNGEGVFKTFLEHLDKALPFEKEVGLVEQGLQKKLYAFSQGNMRSLRNLIYQASVEAIDKQHETITEQDLIFASKLTSGDKSDRWENPFEKGVKVTEGMLRSPPKDIGSGPAAKKKKLDGSGGWEDYYHHVTSLNAKRNGGNMFE

SEQ ID NO: 125 - protein, 394 aa, Vibrio chagasii
MLLQRPKPHSNESLESFFIRVANKNGYEDVNRFLMATKRYLQDIDFSGFQTFPTNICKINPASAKSSSSARIASLLKLAQLTFNEPPDLLGLAINRTNLKYSPSTSAVIRGSEVFPRSLLRTKSIPCCPLCLQQNDYASYLWHFEGYDHCHIHDAPLLNSCRCGAEYDYRVSGLSGMCGECKKTISTKSSENSHKATSTVSSWLAGNESKDLPDVPKSYRWGLIHWWVHISKNEFDHVSFIQFFSKWPSSFHSMIDNEIEFNLEHAIVGRRELRIKDLLGRIFFSSVRLPERNLQHNIVLGELLRHTEMHLWDNNGLIANLRMNALETTVFLNCSKDELASMVEQRILKPNRKTKPNMPLAVNDYLFYFGDIFCLWLAEFQTDEFNRSFYVSRW

SEQ ID NO: 126 - protein, 437 aa, synthetic construct
MPKKKRKVGSGEQKLISEEDLEQKLISEEDLEQKLISEEDLGSGLLQRPKPHSNESLESFFIRVANKNGYEDVNRFLMATKRYLQDIDFSGFQTFPTNICKINPASAKSSSSARIASLLKLAQLTFNEPPDLLGLAINRTNLKYSPSTSAVIRGSEVFPRSLLRTKSIPCCPLCLQQNDYASYLWHFEGYDHCHIHDAPLLNSCRCGAEYDYRVSGLSGMCGECKKTISTKSSENSHKATSTVSSWLAGNESKDLPDVPKSYRWGLIHWWVHISKNEFDHVSFIQFFSKWPSSFHSMIDNEIEFNLEHAIVGRRELRIKDLLGRIFFSSVRLPERNLQHNIVLGELLRHTEMHLWDNNGLIANLRMNALETTVFLNCSKDELASMVEQRILKPNRKTKPNMPLAVNDYLFYFGDIFCLWLAEFQTDEFNRSFYVSRW

SEQ ID NO: 127 - protein, 230 aa, Vibrio rotiferianus
MLCQYDSFSEDITLALDNAFHNPARKLTKSRGKNIHRYASAKMGKRVTVESALECDACYHFDFEKDIIRFCSQPIRYSYYYNGKWHTYVPDFLVQFDTGEYVLYEIKPDDIASSPDFLDEWSAKQQAAEEMGLELELVEEKQIRNKTLLKNLKLMYRYASRDCLTDTHNLVLNILRDNGPQSAQHLIHKAGLTRRAIMPVLCNLLSRNLLETELDSPLSLKSEFKVNCYA

SEQ ID NO: 128 - protein, 608 aa, Vibrio rotiferianus
MPKKSFSNFNRKAKLEVSDYQEDLVDIDSSLNDALAEDITYKDLTAFPDKVANEISYRLKVLKYLGKECDKITPKTIEPHRVELQRCNDKKIPSAITIYRWWLNFSQSDFNPTCLAPDFKGRGNREPKVPKIVDALMEQAVEGVISGKKINISSAYRRVRRKVRQYNVKNGTKHKYPKYESLRKRVNKKTPFEILSAKKGVRVAKREFRKMGKKILTSYALERVEVDHTVLDVFVVHEEYRIPLGRPYLTQLVDCYSKAVVGFYLGFEPPSYVSVSLALKNAIQRKDSLLSSYPSVKNEWLCYGIMDLLVTDNGKEFLSKAFDAACETLLITVHQNKVETPDNKPHVERNYGTVNTNVLDDLPGKAFSHYIQREGYDSIGEATLTLSELKEVYLIWLVDKYHRKPNQRGTNCPNVAWKRGCEEWEPEEFTGTAAELDFKFAILDKKKLNKSGITVYVDLTYTSDRLAEYRGRKGNHVVTFKYNPECMGHIWVLDEDANEYFTVPAIDYEYASSISLWQHKFNIKYQRNLNSADYDEDAEIDAEIRMEEVAEESIVKTKKIRNRRRGARYQENTERAKSQNQKSLEKAEQGHHQEEDVYDENAWGIDYL

SEQ ID NO: 129 - protein, 880 aa, synthetic construct
MLCQYDSFSEDITLALDNAFHNPARKLTKSRGKNIHRYASAKMGKRVTVESALECDACYHFDFEKDIIRFCSQPIRYSYYYNGKWHTYVPDFLVQFDTGEYVLYEIKPDDIASSPDFLDEWSAKQQAAEEMGLELELVEEKQIRNKTLLKNLKLMYRYASRDCLTDTHNLVLNILRDNGPQSAQHLIHKAGLTRRAIMPVLCNLLSRNLLETELDSPLSLKSEFKVNCYAGSGPKKKRKVGSGYPYDVPDYAYPYDVPDYAYPYDVPDYAGSGPKKSFSNFNRKAKLEVSDYQEDLVDIDSSLNDALAEDITYKDLTAFPDKVANEISYRLKVLKYLGKECDKITPKTIEPHRVELQRCNDKKIPSAITIYRWWLNFSQSDFNPTCLAPDFKGRGNREPKVPKIVDALMEQAVEGVISGKKINISSAYRRVRRKVRQYNVKNGTKHKYPKYESLRKRVNKKTPFEILSAKKGVRVAKREFRKMGKKILTSYALERVEVDHTVLDVFVVHEEYRIPLGRPYLTQLVDCYSKAVVGFYLGFEPPSYVSVSLALKNAIQRKDSLLSSYPSVKNEWLCYGIMDLLVTDNGKEFLSKAFDAACETLLITVHQNKVETPDNKPHVERNYGTVNTNVLDDLPGKAFSHYIQREGYDSIGEATLTLSELKEVYLIWLVDKYHRKPNQRGTNCPNVAWKRGCEEWEPEEFTGTAAELDFKFAILDKKKLNKSGITVYVDLTYTSDRLAEYRGRKGNHVVTFKYNPECMGHIWVLDEDANEYFTVPAIDYEYASSISLWQHKFNIKYQRNLNSADYDEDAEIDAEIRMEEVAEESIVKTKKIRNRRRGARYQENTERAKSQNQKSLEKAEQGHHQEEDVYDENAWGIDYL

SEQ ID NO: 130 - protein, 330 aa, Vibrio rotiferianus
MDDSRDIRIARAKKAFVITPSVAKVLRYMDRCRDFSDMDSEPTCMIVYGASGVGKTTIIKKYLKKNEGDSDIDGDTIPVVHIELPDNAKPVDAARELLLKMGDPLALYDTDLARLTKRIVELIPALGVKLIIIDEFQHLVEESSNKILTQVGNWLKGILNKSKCPIVLFGMPYSKLVLQANSQLHGRFSIQFDLRPFSYQEGEGTFKTFLQHLDEALPFEKQAGLANEGLQKKLYAFSQGNMRSLRDLIYHASIEAIDNHHESITKDDFLFASQLTSGNKSTFWKNPFIEGVKVTKDMLRSPPKSIGWEDYYQQNNSRKKKGKGRPDFFD

SEQ ID NO: 131 - protein, 345 aa, synthetic construct
MDDSRDIRIARAKKAFVITPSVAKVLRYMDRCRDFSDMDSEPTCMIVYGASGVGKTTIIKKYLKKNEGDSDIDGDTIPVVHIELPDNAKPVDAARELLLKMGDPLALYDTDLARLTKRIVELIPALGVKLIIIDEFQHLVEESSNKILTQVGNWLKGILNKSKCPIVLFGMPYSKLVLQANSQLHGRFSIQFDLRPFSYQEGEGTFKTFLQHLDEALPFEKQAGLANEGLQKKLYAFSQGNMRSLRDLIYHASIEAIDNHHESITKDDFLFASQLTSGNKSTFWKNPFIEGVKVTKDMLRSPPKSIGSGPAAKKKKLDGSGGWEDYYQQNNSRKKKGKGRPDFFD

SEQ ID NO: 132 - protein, 395 aa, Vibrio rotiferianus
MLLQRPKTYPDESLESFFIRVANKNGYDDIQRFLEALKRFLIDKNPRQFQTFPTNICKINPYSSKNHSISRTNALLELSHMTFNEPANLLGMALNRNQMKFSPSTTALIRGAEVIPRSLLLKDSVPCCPMCLHEKGYANYRWHFSGYDYCHEHNVKLVSHCTCGSTYDYRTAGLSGICPECGDIIASAQVHDDSSGVKIASWLSGFDVDPLPIIPQSYRWGLIHWWSQMFGATQTSDSEKFVTFWEQWPNSFHDMIETEIETGFEYAVVSHTELRIKNVLGKILFSSIKLPDRNFRSNIILKELFQYLEAHLWDNDGRLANLRLNTSDICIVLNCSKEQVASMVEQRILIPTRHPKSRGILIDTNYVYYFGDIYCLWLSEFQTDEFNRSFYVSRW

SEQ ID NO: 133 - protein, 438 aa, synthetic construct
MPKKKRKVGSGEQKLISEEDLEQKLISEEDLEQKLISEEDLGSGLLQRPKTYPDESLESFFIRVANKNGYDDIQRFLEALKRFLIDKNPRQFQTFPTNICKINPYSSKNHSISRTNALLELSHMTFNEPANLLGMALNRNQMKFSPSTTALIRGAEVIPRSLLLKDSVPCCPMCLHEKGYANYRWHFSGYDYCHEHNVKLVSHCTCGSTYDYRTAGLSGICPECGDIIASAQVHDDSSGVKIASWLSGFDVDPLPIIPQSYRWGLIHWWSQMFGATQTSDSEKFVTFWEQWPNSFHDMIETEIETGFEYAVVSHTELRIKNVLGKILFSSIKLPDRNFRSNIILKELFQYLEAHLWDNDGRLANLRLNTSDICIVLNCSKEQVASMVEQRILIPTRHPKSRGILIDTNYVYYFGDIYCLWLSEFQTDEFNRSFYVSRW

SEQ ID NO: 134 - protein, 207 aa, Vibrio parahaemolyticus
MKKRIIKNSKVKNISRFVSLKTDSVQTTESDLEFDACFHFEFASHVKSFETQPLGFEYRLNGRLRRYTPDMLCYFNDGYATYYEVKPKWVTERDEFKKKFDAQKQQAIANGYDLLVLTEDDIQTYPLLDNLKIIHRYACSDSLDDVQVRILKLFQNYGEMRISQVINASQGQSASILPALYDLIAKKILEFDWHCPISHDSLVWRVS

SEQ ID NO: 135 - protein, 624 aa, Vibrio parahaemolyticus
MSDDSENLYAFGSFFPEKHSNTSVPKTSKGTRFGIELQESYQDLFSFDEKRRDEAIHRYNILDYLIELHGPSLTLKKISGSMKGLADKFHPNVPSAPSIYRYWTTFKKSGFVLSSLIPGVTRGNTKQRKTLELEEYIERAIKSYFSAESPTIQQAFTLLETEIDRHNECNDTQLSFEYESFRKRIVKKTDYERLLIKKGKKAADTYYKKVGQRPETTRVLQRVEADHTRLDLFVIDDARKLPLGRPWLTLLFDTHTKSVVGFYLGFEPPGYLSVSLALENAILPKYYVKELYPEVKGEWPCYGLPEHLIVDNGAEFNSKDFVTACKNLRIKVKKNPVKKPWLKGSVERYFRTINNKLLSGIPGKSFSNIFARGDYNPQKNAIITRSDLMKVIHVWLIDIYQSSPNGLENNIPNLSWADAMRSAFPPRSFNGSIDELRFNLGKHVEISLDRNGIRLKKTLRYTSSYLAQYFGKHTYDGKSIKVKIKYNPICMGSIYVLDEDKHEFFAVESVDPDYAYSVSEWLHKVCCDYARNHIRNNYRHNDVIKAWRVIYDIIDEALHLSGNGKQANVGIRQASKLERVREHAERTKSHQKPELHMSSNDDIDWDVEVNTDGWKIDSVRGTNK

SEQ ID NO: 136 - protein, 873 aa, synthetic construct
MKKRIIKNSKVKNISRFVSLKTDSVQTTESDLEFDACFHFEFASHVKSFETQPLGFEYRLNGRLRRYTPDMLCYFNDGYATYYEVKPKWVTERDEFKKKFDAQKQQAIANGYDLLVLTEDDIQTYPLLDNLKIIHRYACSDSLDDVQVRILKLFQNYGEMRISQVINASQGQSASILPALYDLIAKKILEFDWHCPISHDSLVWRVSGSGPKKKRKVGSGYPYDVPDYAYPYDVPDYAYPYDVPDYAGSGSDDSENLYAFGSFFPEKHSNTSVPKTSKGTRFGIELQESYQDLFSFDEKRRDEAIHRYNILDYLIELHGPSLTLKKISGSMKGLADKFHPNVPSAPSIYRYWTTFKKSGFVLSSLIPGVTRGNTKQRKTLELEEYIERAIKSYFSAESPTIQQAFTLLETEIDRHNECNDTQLSFEYESFRKRIVKKTDYERLLIKKGKKAADTYYKKVGQRPETTRVLQRVEADHTRLDLFVIDDARKLPLGRPWLTLLFDTHTKSVVGFYLGFEPPGYLSVSLALENAILPKYYVKELYPEVKGEWPCYGLPEHLIVDNGAEFNSKDFVTACKNLRIKVKKNPVKKPWLKGSVERYFRTINNKLLSGIPGKSFSNIFARGDYNPQKNAIITRSDLMKVIHVWLIDIYQSSPNGLENNIPNLSWADAMRSAFPPRSFNGSIDELRFNLGKHVEISLDRNGIRLKKTLRYTSSYLAQYFGKHTYDGKSIKVKIKYNPICMGSIYVLDEDKHEFFAVESVDPDYAYSVSEWLHKVCCDYARNHIRNNYRHNDVIKAWRVIYDIIDEALHLSGNGKQANVGIRQASKLERVREHAERTKSHQKPELHMSSNDDIDWDVEVNTDGWKIDSVRGTNK

SEQ ID NO: 137 - protein, 317 aa, Vibrio parahaemolyticus
VNHFARAPHQQVKSIFISNSQIDEILSDIEECREESDGISEPECLIVVGDSGSGKTTIIDKYLVDNPRMEANDGSIIPILFTSLPANANPVTASERLLSSMGDPLAFSHGKDPAELMKIVNDLLRECRVELIIIDEFQHMIDRKSKDVLHSTADWLKMIIIDSKIPVVLFGMPYSTEILRVNNQLRGRFESQHHLKPFRVKDTSELIRYKTFMTMLDAALPFLEESGLASEDIMKRVYIFSKGNMRLIRRLINKAAKFALLENAPCISLKHFARAAPKVSRDACKSFNPFDTDTKKLKIIEPPEDVGWENYLAAKGD

SEQ ID NO: 138 - protein, 332 aa, synthetic construct
MNHFARAPHQQVKSIFISNSQIDEILSDIEECREESDGISEPECLIVVGDSGSGKTTIIDKYLVDNPRMEANDGSIIPILFTSLPANANPVTASERLLSSMGDPLAFSHGKDPAELMKIVNDLLRECRVELIIIDEFQHMIDRKSKDVLHSTADWLKMIIIDSKIPVVLFGMPYSTEILRVNNQLRGRFESQHHLKPFRVKDTSELIRYKTFMTMLDAALPFLEESGLASEDIMKRVYIFSKGNMRLIRRLINKAAKFALLENAPCISLKHFARAAPKVSRDACKSFNPFDTDTKKLKIIEPPEDVGSGPAAKKKKLDGSGGWENYLAAKGD

SEQ ID NO: 139 - protein, 396 aa, Vibrio parahaemolyticus
MMLLQRPKSYPDESLESFFIRVANKNGYNDVHWFLVAVKRYLLDIDPRKFQTFPTDICCINPYSSKKHSISRTHALHHLSQLTFNEPVDLLGIALNRNQMQFSPSTTALIRGAEVIPRSLLRKGAIPCCPCCLGEHGYASYRWHFSGYEYCHEHDVKLIERCSCGAIYDYRYAGLSGVCTECGENISASQENHEPKATRIASWLAGDDVKPLPDVPLSYRWGFMHWWSQISSSCKTRNNGEFLAFWEHWPNSFHKLIGKEIDFNFEYCVLSKNDLRVKDILGKILFSSIQLPDRNFRSNIILKEMFQYIETHLWDDNGKLANLRMNMLEICVLLNCSREQVTSMIEQGLLPPNRQLGKREILIVTEYAFYLGDVYCLWLSEFQSDEFNRSFYLSRW

SEQ ID NO: 140 - protein, 439 aa, synthetic construct
MPKKKRKVGSGEQKLISEEDLEQKLISEEDLEQKLISEEDLGSGMLLQRPKSYPDESLESFFIRVANKNGYNDVHWFLVAVKRYLLDIDPRKFQTFPTDICCINPYSSKKHSISRTHALHHLSQLTFNEPVDLLGIALNRNQMQFSPSTTALIRGAEVIPRSLLRKGAIPCCPCCLGEHGYASYRWHFSGYEYCHEHDVKLIERCSCGAIYDYRYAGLSGVCTECGENISASQENHEPKATRIASWLAGDDVKPLPDVPLSYRWGFMHWWSQISSSCKTRNNGEFLAFWEHWPNSFHKLIGKEIDFNFEYCVLSKNDLRVKDILGKILFSSIQLPDRNFRSNIILKEMFQYIETHLWDDNGKLANLRMNMLEICVLLNCSREQVTSMIEQGLLPPNRQLGKREILIVTEYAFYLGDVYCLWLSEFQSDEFNRSFYLSRW

SEQ ID NO: 141 - protein, 229 aa, Vibrio parahaemolyticus
MLCQDSFSENVVLALEQAFHNPARKLTKSRGKNIHRFASAKMGKRVTVESALECDACYCFDFEKDIIRFCAQPIRYSYYYNGKWRTYVPDFLVQFDTGEYVLYEVKPDNIASSSDFLDEWNAKQQAAQTRGLELELVEEKQIRVKNLLKNLKLMHRYASRDCLSDKHNLVLNILRKNGSQSAQYLSDKTGLSRRAIMPVLCNLLSRNLLETDLDTPISFQSEFELVSYG

SEQ ID NO: 142 - protein, 608 aa, Vibrio parahaemolyticus
MAKKSFSNFNRKAKRDDVSHQEETLYIDRALNDTLDEDATYTDLTAFPDKVAIEISFRLKILRYLGRVNDKIVPKTIEPHRVTLQRCNDKNIPSAITIYRWWLNFSQSGYNPTSLAPKFKGRGNRAPKVSEIVDALMAQAVEAVISGRKINVSSAHRRVRRKVRQYNLKHGTKYKYPRYESVRKRVKKKTPFEVLVAKKGERVAKREFRRMGKKILTSYALERVEVDHTVVDLFAVHKEYRLPLGRPYLTQLVDCYSKAVVGFYLGFEPPSYVSVALALKNAIQRKDSLLSSYPTVKNEWLCYGIPDLLVTDNGKEFLSKAFDAACETLLITVHQNKVDTPDNKPDVERKYGTVNTTLLDDLPGKAFSQYLHREGYDSIDEATLTLDEIKEIYLIWLVDMYHKHPNQRGTNCPNVAWKRGCEEWEPEEFTGTTAELDFKFAVLDEKKLSKSGITVYVDLTYSSDRLAEYRGTHGNHMVTFKYNPECMGVIWVLDEDVDEYFTVPAIDYDYASGVSLWQHKYNIKYQRSLNLSEYDEDFEVDAEIRIEDIAEESIVKTKKLRNRRRGARYQENAERAKAQNQNAIIKTEQEDPQEEEVDDENAWGIDYL

SEQ ID NO: 143 - protein, 879 aa, synthetic construct
MLCQDSFSENVVLALEQAFHNPARKLTKSRGKNIHRFASAKMGKRVTVESALECDACYCFDFEKDIIRFCAQPIRYSYYYNGKWRTYVPDFLVQFDTGEYVLYEVKPDNIASSSDFLDEWNAKQQAAQTRGLELELVEEKQIRVKNLLKNLKLMHRYASRDCLSDKHNLVLNILRKNGSQSAQYLSDKTGLSRRAIMPVLCNLLSRNLLETDLDTPISFQSEFELVSYGGSGPKKKRKVGSGYPYDVPDYAYPYDVPDYAYPYDVPDYAGSGAKKSFSNFNRKAKRDDVSHQEETLYIDRALNDTLDEDATYTDLTAFPDKVAIEISFRLKILRYLGRVNDKIVPKTIEPHRVTLQRCNDKNIPSAITIYRWWLNFSQSGYNPTSLAPKFKGRGNRAPKVSEIVDALMAQAVEAVISGRKINVSSAHRRVRRKVRQYNLKHGTKYKYPRYESVRKRVKKKTPFEVLVAKKGERVAKREFRRMGKKILTSYALERVEVDHTVVDLFAVHKEYRLPLGRPYLTQLVDCYSKAVVGFYLGFEPPSYVSVALALKNAIQRKDSLLSSYPTVKNEWLCYGIPDLLVTDNGKEFLSKAFDAACETLLITVHQNKVDTPDNKPDVERKYGTVNTTLLDDLPGKAFSQYLHREGYDSIDEATLTLDEIKEIYLIWLVDMYHKHPNQRGTNCPNVAWKRGCEEWEPEEFTGTTAELDFKFAVLDEKKLSKSGITVYVDLTYSSDRLAEYRGTHGNHMVTFKYNPECMGVIWVLDEDVDEYFTVPAIDYDYASGVSLWQHKYNIKYQRSLNLSEYDEDFEVDAEIRIEDIAEESIVKTKKLRNRRRGARYQENAERAKAQNQNAIIKTEQEDPQEEEVDDENAWGIDYL

SEQ ID NO: 144 - protein, 331 aa, Vibrio parahaemolyticus
MDDSRDIRVAKAKKAFVITPSVAKVLRYMDRCRDLSDMDSEPTCMMVYGSHGVGKTAIIKKYLKQNEGDSDTEGDTIPVIHIEMPDNAKPVDAARELLLQMEDPLALYDTDLARLTKRIVELIPLLGVKLIIIDEFQHLVDESSNKILTQVGNWLKGILNKSKCPIVLFGMPYSKLVLQANSQLHSRFSIQFNLRPFNYQEGEGTFKTFLQHLDEALPFEKQTGLAKEGLQEKLYAFSQGNMRSLRDLIYQASIEAIDNHHESITKDDFLFASQLTSGNKPTFWKNPFIEGVKVTKEMLRSPPRSIGWEDYYQQNNSRKKKGKGRPDFFDK

SEQ ID NO: 145 - protein, 346 aa, synthetic construct
MDDSRDIRVAKAKKAFVITPSVAKVLRYMDRCRDLSDMDSEPTCMMVYGSHGVGKTAIIKKYLKQNEGDSDTEGDTIPVIHIEMPDNAKPVDAARELLLQMEDPLALYDTDLARLTKRIVELIPLLGVKLIIIDEFQHLVDESSNKILTQVGNWLKGILNKSKCPIVLFGMPYSKLVLQANSQLHSRFSIQFNLRPFNYQEGEGTFKTFLQHLDEALPFEKQTGLAKEGLQEKLYAFSQGNMRSLRDLIYQASIEAIDNHHESITKDDFLFASQLTSGNKPTFWKNPFIEGVKVTKEMLRSPPRSIGSGPAAKKKKLDGSGGWEDYYQQNNSRKKKGKGRPDFFDK

SEQ ID NO: 146 - protein, 395 aa, Vibrio parahaemolyticus
MLLQRPKPYPDESLESFFIRVANKNGYSDVNWFLLAVKRYLLGIDPRKFQTFPTDICRINPHSSKKHSISRTHALHHLSQLTFNEPVDLLGIALNRNQMQFSTSTTAVIRGAEVIPRSLLRKGVIPCCPSCLGEHGYASYRWHFSGYEYCHEHDVKLIERCSCGAVYDYRYAGLSGVCTECGENISASQENHEPKATRIASWLAGDDVKPLPDVPLSYRWGFMHWWSQISSSCKTRNNGEFLAFWEHWPNSFHKLIGKEIDFNFEYCVLSKNDLRVKDILGKILFSSIQLPDRNFRSNIILKEMFQYIETHLWSDNGRLANLRVNTLEICVLLNCSREQVTSMIEQGLLRPNRQLGKQETLIVTEYAFYLGDVYCLWLSEFQSDEFNRSFYLSRW

SEQ ID NO: 147 - protein, 438 aa, synthetic construct
MPKKKRKVGSGEQKLISEEDLEQKLISEEDLEQKLISEEDLGSGLLQRPKPYPDESLESFFIRVANKNGYSDVNWFLLAVKRYLLGIDPRKFQTFPTDICRINPHSSKKHSISRTHALHHLSQLTFNEPVDLLGIALNRNQMQFSTSTTAVIRGAEVIPRSLLRKGVIPCCPSCLGEHGYASYRWHFSGYEYCHEHDVKLIERCSCGAVYDYRYAGLSGVCTECGENISASQENHEPKATRIASWLAGDDVKPLPDVPLSYRWGFMHWWSQISSSCKTRNNGEFLAFWEHWPNSFHKLIGKEIDFNFEYCVLSKNDLRVKDILGKILFSSIQLPDRNFRSNIILKEMFQYIETHLWSDNGRLANLRVNTLEICVLLNCSREQVTSMIEQGLLRPNRQLGKQETLIVTEYAFYLGDVYCLWLSEFQSDEFNRSFYLSRW

SEQ ID NO: 148 - protein, 206 aa, Vibrio fischeri
MYVRTLKQSQVKNISKFMSLKNDSIIRTESMLEFDMCFHLEYSPDVVSFESQPQGFYYKYQGKHLPYTPDFLITHSSGLQQLLEIKPLSKTQRPDFQSKFIQKQQAAQKLNLSLILITEKQIRTGHLLNNFKLLHRYSGLHSISATQKAIINLIQKVNKIQISQIANSLNISNGEALTGVLSWLSKGALQTDYSNEAITGNSYVWL

SEQ ID NO: 149 - protein, 616 aa, Vibrio fischeri
MNFPFDDEFQKIINISGEQNKVIRNEEANSIQLSLDSYSHDIKMEVLRRISFIKWIKPRLKGGLTEKNLKPLLSDASIHLKMKAPCTSTFIAWCNRYRLSGEKVSSLIPQHSQKGNRKLKTSSEFYIAKAINEKYLTRNQCSIIQAFKYYCDLIIIENRSTPTNKIKKISQRTFYNRINALPKYEVALKRYGKRYADINYRKVGKIREATRPLEYVEIDHTPLDLILLDDELEIPLGRPYLTILIDRYSKCIIGYNISFRPPSFESIRHAFCNACLDKSSITQQYPHLKNDWPMAGKIENLVVDNGAEFWSNSLEDSLRPFATNILFNKVGKPWMKPLVEKFFDVLNKELVHSLPGTTRSRVEQLKGYNPKKDAAITFSLFLELFHTWIIDIYHMTPDTRGVSIPYFKWQEGIKNLPPLSFSNEEAQQLLIEFGILNTRTLTIHGISIHNKRYQSDELIEYRKKYGNIKENNLRLKTKTNPSNISYIFVYLPNEARYIKVPCTDGDSYIKNLTLYQHNVISKLTRTKTSLQENKEDQADSRMYIDKRIGKQLEKIQENKKNIGKIKHISKIACYQNIGSHTQKSLQFPTLNDNTKSEYKDRILNNWNEQFDDLEGF

SEQ ID NO: 150 - protein, 864 aa, synthetic construct
MYVRTLKQSQVKNISKFMSLKNDSIIRTESMLEFDMCFHLEYSPDVVSFESQPQGFYYKYQGKHLPYTPDFLITHSSGLQQLLEIKPLSKTQRPDFQSKFIQKQQAAQKLNLSLILITEKQIRTGHLLNNFKLLHRYSGLHSISATQKAIINLIQKVNKIQISQIANSLNISNGEALTGVLSWLSKGALQTDYSNEAITGNSYVWLGSGPKKKRKVGSGYPYDVPDYAYPYDVPDYAYPYDVPDYAGSGNFPFDDEFQKIINISGEQNKVIRNEEANSIQLSLDSYSHDIKMEVLRRISFIKWIKPRLKGGLTEKNLKPLLSDASIHLKMKAPCTSTFIAWCNRYRLSGEKVSSLIPQHSQKGNRKLKTSSEFYIAKAINEKYLTRNQCSIIQAFKYYCDLIIIENRSTPTNKIKKISQRTFYNRINALPKYEVALKRYGKRYADINYRKVGKIREATRPLEYVEIDHTPLDLILLDDELEIPLGRPYLTILIDRYSKCIIGYNISFRPPSFESIRHAFCNACLDKSSITQQYPHLKNDWPMAGKIENLVVDNGAEFWSNSLEDSLRPFATNILFNKVGKPWMKPLVEKFFDVLNKELVHSLPGTTRSRVEQLKGYNPKKDAAITFSLFLELFHTWIIDIYHMTPDTRGVSIPYFKWQEGIKNLPPLSFSNEEAQQLLIEFGILNTRTLTIHGISIHNKRYQSDELIEYRKKYGNIKENNLRLKTKTNPSNISYIFVYLPNEARYIKVPCTDGDSYIKNLTLYQHNVISKLTRTKTSLQENKEDQADSRMYIDKRIGKQLEKIQENKKNIGKIKHISKIACYQNIGSHTQKSLQFPTLNDNTKSEYKDRILNNWNEQFDDLEGF

SEQ ID NO: 151 - protein, 336 aa, Vibrio fischeri
MHALSSAQKEQLINFNQCFIEYPIITHIYSIFDDLRLNQGLGAEPQCMLLLGDTGSGKSALINNYLLQQPSSNFSALSSLPVLHTRIPRRVNNEQTMYQLLTDLGQSPSGTRRTKRSEIALAEGVVRALKRKKTELIIINEFQELIEFSSARERQNVANTLKYISEEARVSIVLVGMPYADIIAKEPQWGSRLAWKTQIEYFSLKNDMKTYVQFLKGLANRMGYAEVPSLHSKELAIPLFSICRGELRQLKNFCSDAMLESFKQNKNTLTHYVLSATFKYKYPTKKNPFEMNVEDIPIQEVISYSKYNLDEMDDNKRLISTKYSDALPLTVILSQS

SEQ ID NO: 152 - protein, 351 aa, synthetic construct
MHALSSAQKEQLINFNQCFIEYPIITHIYSIFDDLRLNQGLGAEPQCMLLLGDTGSGKSALINNYLLQQPSSNFSALSSLPVLHTRIPRRVNNEQTMYQLLTDLGQSPSGTRRTKRSEIALAEGVVRALKRKKTELIIINEFQELIEFSSARERQNVANTLKYISEEARVSIVLVGMPYADIIAKEPQWGSRLAWKTQIEYFSLKNDMKTYVQFLKGLANRMGYAEVPSLHSKELAIPLFSICRGELRQLKNFCSDAMLESFKQNKNTLTHYVLSATFKYKYPTKKNPFEMNVEDIPIQEVISYSGSGPAAKKKKLDGSGKYNLDEMDDNKRLISTKYSDALPLTVILSQS

SEQ ID NO: 153 - protein, 399 aa, Vibrio fischeri
MLNPIELYEDESLESCLLRISQNNYYDSFQDFSDEVWFHVKEEDREVRGTFPATLNTVNLYHSHTSSDLKLKALIKIEQWLEINNFPLLKSALSRSSNTFLRQHSAVFRNGVDIPRILLRKNGIPVCPECLKENEYIRQEWHFITHDVCTKHKTDLLHHCPECKTSINYQESENITDCQCGFKFSDHLTPQANSNALLIAQWLNSENTKLANVWGEHQAISSRFGVLLWYINRYNLTDDFSTSFVKYSLNWPTNFYSELDEQIDKAKTVQIKPFNKIFFNEIFNRLLLDCRHLPTREFKTNSILSHIYQYFLSRYQIQPNSGVFSILLSPLEASTLLSCTTDQIYRLYELGFLKLGVRPKLHQKIASHQSVFTLSSIILVKLSNMQSSQDELHHYLSAW

SEQ ID NO: 154 - protein, 442 aa, synthetic construct
MPKKKRKVGSGEQKLISEEDLEQKLISEEDLEQKLISEEDLGSGLNPIELYEDESLESCLLRISQNNYYDSFQDFSDEVWFHVKEEDREVRGTFPATLNTVNLYHSHTSSDLKLKALIKIEQWLEINNFPLLKSALSRSSNTFLRQHSAVFRNGVDIPRILLRKNGIPVCPECLKENEYIRQEWHFITHDVCTKHKTDLLHHCPECKTSINYQESENITDCQCGFKFSDHLTPQANSNALLIAQWLNSENTKLANVWGEHQAISSRFGVLLWYINRYNLTDDFSTSFVKYSLNWPTNFYSELDEQIDKAKTVQIKPFNKIFFNEIFNRLLLDCRHLPTREFKTNSILSHIYQYFLSRYQIQPNSGVFSILLSPLEASTLLSCTTDQIYRLYELGFLKLGVRPKLHQKIASHQSVFTLSSIILVKLSNMQSSQDELHHYLSAW

SEQ ID NO: 155 - protein, 229 aa, Vibrio parahaemolyticus
MSALPSPSTATLIALESAFDTPARNLTKSRGKNIHRYVSAKMGKRVTVESFLECAACYHFDFEPSIVRFCSQPIRLSYCLNGKAHTYVPDFLVQFDTGEYTLYEVKSDMESSKSEFQCEWEAKVQGAFELGLELELVTEEEILDEVIFSNLRLLHRYASRDNLNHFHQTLLTTLKLNGTQTAKSLGHHLGLNERKIFPFLCDLLSRNLLQTSLETPLSLESEFELGCYA

SEQ ID NO: 156 - protein, 603 aa, Vibrio parahaemolyticus
MPKKSFSSFHRKSALQQEKPEPDERVVDTSDVDEETYRDISAFPDNIATQITFRLSILRYLASKCEKIIPKTIEPHRVALQRLHDRNIPSAISIYRWWLVFRASDCNPVSLAPRNKDKGNSKVKLPKFVDALMKQAVERVISGRKVRIRSAYKRVRRKLRQHNLNNGTKYKYPTYESLRKRVKKKTPFELLAAKKGERVAKREFRRMGKKILTSYVLERVEIDHTVVDLFAVHEEHRVPLGRPWLTQLVDCYSKAVIGFYLGFEPPSYVSVSLALKNAILRKDDLLSSFDSVENEWLCYGIPDLLVTDNGKEFLSKAFDKACESLLINVHQNRVETPDNKPHVERNYGTINTSLLDDLPGKAFSQYLHREGYDSVGEATLTLDEIKEIYLIWLVDIYHKNSNQRGTNCPNVAWKRGSQEWEPEEFTGSKDELDFKFAIVEHKQLTKAGVTVYKELTYSSERLAEYRGKKGNHKVQFKYNPECMAVIWVLDEDQNEYFTVNAIDYEYASRVSLWQHKYNMKYQAELNSAEYDEDKEIDADIKIEEIADRSIVKTNKIRARRRGARHQENSARAKSISDAKPVPPQKHEEETVIFDNEDWDIDYV

SEQ ID NO: 157 - protein, 874 aa, synthetic construct
MSALPSPSTATLIALESAFDTPARNLTKSRGKNIHRYVSAKMGKRVTVESFLECAACYHFDFEPSIVRFCSQPIRLSYCLNGKAHTYVPDFLVQFDTGEYTLYEVKSDMESSKSEFQCEWEAKVQGAFELGLELELVTEEEILDEVIFSNLRLLHRYASRDNLNHFHQTLLTTLKLNGTQTAKSLGHHLGLNERKIFPFLCDLLSRNLLQTSLETPLSLESEFELGCYAGSGPKKKRKVGSGYPYDVPDYAYPYDVPDYAYPYDVPDYAGSGPKKSFSSFHRKSALQQEKPEPDERVVDTSDVDEETYRDISAFPDNIATQITFRLSILRYLASKCEKIIPKTIEPHRVALQRLHDRNIPSAISIYRWWLVFRASDCNPVSLAPRNKDKGNSKVKLPKFVDALMKQAVERVISGRKVRIRSAYKRVRRKLRQHNLNNGTKYKYPTYESLRKRVKKKTPFELLAAKKGERVAKREFRRMGKKILTSYVLERVEIDHTVVDLFAVHEEHRVPLGRPWLTQLVDCYSKAVIGFYLGFEPPSYVSVSLALKNAILRKDDLLSSFDSVENEWLCYGIPDLLVTDNGKEFLSKAFDKACESLLINVHQNRVETPDNKPHVERNYGTINTSLLDDLPGKAFSQYLHREGYDSVGEATLTLDEIKEIYLIWLVDIYHKNSNQRGTNCPNVAWKRGSQEWEPEEFTGSKDELDFKFAIVEHKQLTKAGVTVYKELTYSSERLAEYRGKKGNHKVQFKYNPECMAVIWVLDEDQNEYFTVNAIDYEYASRVSLWQHKYNMKYQAELNSAEYDEDKEIDADIKIEEIADRSIVKTNKIRARRRGARHQENSARAKSISDAKPVPPQKHEEETVIFDNEDWDIDYV

SEQ ID NO: 158 - protein, 335 aa, Vibrio parahaemolyticus
MLRNHQMNETREARISKAKRAFVSTPSVTKILCYMDRCRDLSDFDSEPTCMMVYGASGVGKTTIIKKYLNQNRRDSDVGGDVIPVLHIELPDNAKPVDAARELLVEMGDPLAIYETDLARLTKKLVDLIPVVGVKLIIIDEFQHLVEERSNRVLTQVGNWLKRILNKTKCPIVLFGMPYSKVVLQANSQLHGRFSIQFELRPFSYQGGKGVFNTFLEYLDKALPFERQAGLANESLQKKLYAFSQGNMRSLRNLIYQASIEAIDNQHATITEEDFVFASKLTSGDKPITWKNPFDEGVKVTEDMLRPPPKDIGWEDYYHNVKPKNQRRKGGNIFE

SEQ ID NO: 159 - protein, 350 aa, synthetic construct
MLRNHQMNETREARISKAKRAFVSTPSVTKILCYMDRCRDLSDFDSEPTCMMVYGASGVGKTTIIKKYLNQNRRDSDVGGDVIPVLHIELPDNAKPVDAARELLVEMGDPLAIYETDLARLTKKLVDLIPVVGVKLIIIDEFQHLVEERSNRVLTQVGNWLKRILNKTKCPIVLFGMPYSKVVLQANSQLHGRFSIQFELRPFSYQGGKGVFNTFLEYLDKALPFERQAGLANESLQKKLYAFSQGNMRSLRNLIYQASIEAIDNQHATITEEDFVFASKLTSGDKPITWKNPFDEGVKVTEDMLRPPPKDIGSGPAAKKKKLDGSGGWEDYYHNVKPKNQRRKGGNIFE

SEQ ID NO: 160 - protein, 394 aa, Vibrio parahaemolyticus
MLLQRPKPHSDESLESYLIRVANKNGYESTGRFLISLKSYLCDIDSHRFASFPTDIRLIHPYSSQRSSSTRSHALQHISQLTFTEAPELLGLAISRSPLKYSPSTTSLIRADEIFPKSLIRTKHVPCCTSCLNEQGYANYLWHFEGYNCCHIHEKPLTYQCECGEPYDYRIYGLKLVCPSCGSILTHQGGEPESTSVEIAQWLAGLTTEPFPEIPASYRWGLIHWWMKIQNTEALDTGSFSTFWQQWPESFHNLIEQTLNHNQEYSVLAPHQWRLKDLVGELLFSSINLPSRNLKYNLPLRELFCYLENHLWEYNGLIANLKLNAFDAATVLNCDTEQIASMAEQGVLVPLWSRKREELISYTDYLFHFGDVFCLWLAEFQTDEFNRSFYTSRW

SEQ ID NO: 161 - protein, 437 aa, synthetic construct
MPKKKRKVGSGEQKLISEEDLEQKLISEEDLEQKLISEEDLGSGLLQRPKPHSDESLESYLIRVANKNGYESTGRFLISLKSYLCDIDSHRFASFPTDIRLIHPYSSQRSSSTRSHALQHISQLTFTEAPELLGLAISRSPLKYSPSTTSLIRADEIFPKSLIRTKHVPCCTSCLNEQGYANYLWHFEGYNCCHIHEKPLTYQCECGEPYDYRIYGLKLVCPSCGSILTHQGGEPESTSVEIAQWLAGLTTEPFPEIPASYRWGLIHWWMKIQNTEALDTGSFSTFWQQWPESFHNLIEQTLNHNQEYSVLAPHQWRLKDLVGELLFSSINLPSRNLKYNLPLRELFCYLENHLWEYNGLIANLKLNAFDAATVLNCDTEQIASMAEQGVLVPLWSRKREELISYTDYLFHFGDVFCLWLAEFQTDEFNRSFYTSRW

SEQ ID NO: 162 - protein, 207 aa, Vibrio cholerae
MYVRNLRKPSATKNVYKFASSKNRSVILCESSLERDCCYHLEYSKDVFSFQSQPEGFYYSSGNKRCPYTPDFLVRNQDGSEYYLEVKPLAKTFSEDFKRSFALKRIAAQHQGKLLILVTDKQIRNGVYLENLNLIHRYSGLVDFSLSSTKIVEELSAAGRMCIRSLADNLKLSIGEVIAVVFRLIGLGRVNVPLDSAINEMSVISVN

SEQ ID NO: 163 - protein, 637 aa, Vibrio cholerae
MVMPFDDEFESINDDTQAEYDSTSEAKLVRKQYLPLDSVTIHERDLSSFSEEQKNKALERYKLISAVAKEISGGWTPKNINPLIDKYGLNLSIKRPSYKSVIRWYKSFCGSDGNIVCLVDHNHSKGNRTKRIIDDESFFVEATERFLDAKRPNYSQAYQFYCDRIEIENSNIISGQISKVSYQAFKERLKKLPPYEVALKRFGPNYANKLFNYYQSSVPTTRILERVELDHTPLDLILLDDDLLIPLGRAYLTLLVDVFSGCIVGFHLGFNPPSYVSVAKAIIHSVKSKDYVHDLNIELTNDWLCHGKMETLVVDNGAEFWSKSLDQACMEAGIHYEYCKVGQPWEKPRVERKFLEIIQGIVGWVPGKTFSNILEKDRYDPQKDAVMRFSSFVEELHRWIIDVHNASPDSRNTKIPNYHWKKSEEALPPAALSDRDEKQFRIIMGVIHEGVVTTKGIKYKHLMYDNVALEQYRKQYPQTKESRKKTIKIDPDDLSSIFVYLEEIGGYIEVPCKYDPLGYTKNLSLSEHVRITKIHRDFIKGQVDALSLAKARQALHERIKTEQEHLSLMSVESRAKKAKHGKKMAALSGISNEQPMSIQNALENKNKPLDDNFDEPTPVDNLKSLWNKRKAMKRSKE

SEQ ID NO: 164 - protein, 886 aa, synthetic construct
MYVRNLRKPSATKNVYKFASSKNRSVILCESSLERDCCYHLEYSKDVFSFQSQPEGFYYSSGNKRCPYTPDFLVRNQDGSEYYLEVKPLAKTFSEDFKRSFALKRIAAQHQGKLLILVTDKQIRNGVYLENLNLIHRYSGLVDFSLSSTKIVEELSAAGRMCIRSLADNLKLSIGEVIAVVFRLIGLGRVNVPLDSAINEMSVISVNGSGPKKKRKVGSGYPYDVPDYAYPYDVPDYAYPYDVPDYAGSGVMPFDDEFESINDDTQAEYDSTSEAKLVRKQYLPLDSVTIHERDLSSFSEEQKNKALERYKLISAVAKEISGGWTPKNINPLIDKYGLNLSIKRPSYKSVIRWYKSFCGSDGNIVCLVDHNHSKGNRTKRIIDDESFFVEATERFLDAKRPNYSQAYQFYCDRIEIENSNIISGQISKVSYQAFKERLKKLPPYEVALKRFGPNYANKLFNYYQSSVPTTRILERVELDHTPLDLILLDDDLLIPLGRAYLTLLVDVFSGCIVGFHLGFNPPSYVSVAKAIIHSVKSKDYVHDLNIELTNDWLCHGKMETLVVDNGAEFWSKSLDQACMEAGIHYEYCKVGQPWEKPRVERKFLEIIQGIVGWVPGKTFSNILEKDRYDPQKDAVMRFSSFVEELHRWIIDVHNASPDSRNTKIPNYHWKKSEEALPPAALSDRDEKQFRIIMGVIHEGVVTTKGIKYKHLMYDNVALEQYRKQYPQTKESRKKTIKIDPDDLSSIFVYLEEIGGYIEVPCKYDPLGYTKNLSLSEHVRITKIHRDFIKGQVDALSLAKARQALHERIKTEQEHLSLMSVESRAKKAKHGKKMAALSGISNEQPMSIQNALENKNKPLDDNFDEPTPVDNLKSLWNKRKAMKRSKE

SEQ ID NO: 165 - protein, 328 aa, Vibrio cholerae
MNLSAKQEIAVDELLTQYHNSFVIYPDVQQIFDGLDWIVRRSQFGNFTPSMLITGGTGAGKTSLINHYAKYHFNDNEVLITRVRPSFIETLIWAIDKLGIPYNTRSKRSEIGLQDYFINSVKKSNLKLLVIEEAQELFECASPKERQKIRDRLKMISDECRLPIVFIGIPTAKLILEDSQWDRRIMVKRELPYIRITSESSLDVYIDLLEELEKQLPISVQPELSEMDIAMRLLSATKGMLGAIKELVGYALELALLSGKSAITNDEFALGFERINGPDVTNPFTTELEKLLVPQVIEYEGFIIDPENGEIKFTKQIFKDIPLAALLG

SEQ ID NO: 166 - protein, 343 aa, synthetic construct
MNLSAKQEIAVDELLTQYHNSFVIYPDVQQIFDGLDWIVRRSQFGNFTPSMLITGGTGAGKTSLINHYAKYHFNDNEVLITRVRPSFIETLIWAIDKLGIPYNTRSKRSEIGLQDYFINSVKKSNLKLLVIEEAQELFECASPKERQKIRDRLKMISDECRLPIVFIGIPTAKLILEDSQWDRRIMVKRELPYIRITSESSLDVYIDLLEELEKQLPISVQPELSEMDIAMRLLSATKGMLGAIKELVGYALELALLSGKSAITNDEFALGFERINGPDVTNPFTTELEKLLVPQVIEYEGSGPAAKKKKLDGSGGFIIDPENGEIKFTKQIFKDIPLAALLG

SEQ ID NO: 167 - protein, 403 aa, Vibrio cholerae
VETDIQLYPDESLESFLLRLSQEQGYERFSHFAEDIWFDTLDQHEAIPGAFPLELNRINIYHAQTTSQMRVRVLIHLENQLKLNNFGALRLALSHSKAQFSPEYKAVHRFEADYPFVFLAKRFTPICPLCISEAPYIRQQWQFLSQQACERHGCKLVHHCPECQSRLEYQTTESISQCECGFELRNSPVEDAPVAALLVARWLSGNDSKPLGLLKAEMTLSERYGFLLWYVNRYGDIENISFESFVEYCSCWPRVLQEELDELVNKADLIRVKDWKKTFFNEVFGALLKDCRQLPSRQLNRNSVLTQVLAYFTKLMATLPSSSKGNVGDVLLSPLEVSTLLSCTTDEVYRLYEFGEIKAAIRPRMHTKIASHESAFTLRSVIETKLTRMCSENDGLSVYLPEW

SEQ ID NO: 168 - protein, 446 aa, synthetic construct
MPKKKRKVGSGEQKLISEEDLEQKLISEEDLEQKLISEEDLGSGETDIQLYPDESLESFLLRLSQEQGYERFSHFAEDIWFDTLDQHEAIPGAFPLELNRINIYHAQTTSQMRVRVLIHLENQLKLNNFGALRLALSHSKAQFSPEYKAVHRFEADYPFVFLAKRFTPICPLCISEAPYIRQQWQFLSQQACERHGCKLVHHCPECQSRLEYQTTESISQCECGFELRNSPVEDAPVAALLVARWLSGNDSKPLGLLKAEMTLSERYGFLLWYVNRYGDIENISFESFVEYCSCWPRVLQEELDELVNKADLIRVKDWKKTFFNEVFGALLKDCRQLPSRQLNRNSVLTQVLAYFTKLMATLPSSSKGNVGDVLLSPLEVSTLLSCTTDEVYRLYEFGEIKAAIRPRMHTKIASHESAFTLRSVIETKLTRMCSENDGLSVYLPEW

SEQ ID NO: 169 - protein, 206 aa, Agarivorans gilvus
MKSRVIGPSTHKSIFKFASPKMGKMVKVESSLEYDACFHFEYSPSITSFIAQPCGVDYQLNGRTQTFYPDFLVEDKEFGKRFFEIKPSSKVRKPEFRVKFALRREAALSQSIPLIVVTEKQICLNPILNNLKLLHRYAGNYSLTPLHFWVLDAVKSLGRITVRDLVDESDCAPGDVFASALTWISRGHLQADISDNELGVNSLVWC

SEQ ID NO: 170 - protein, 631 aa, Agarivorans gilvus
MASSRHTLGLFDDEYDSLSAESIESFSKETSDLDNDHLSPDFDSYSKEQQREALRRYALIQWVDKRLKGGWTEKKLSPLLEQATIEFDFTLPNWRTLSRWYSSYINSGHSLEALLPKHHKKGGTGARKMEDGFFFEKAIEEYYLTRERPTIADCYELYKSWIVLENSKLISGKLKPVCQRTFYNRINKLSPYLVALRRFGKPYADRHFRTVKQLKKPSNVLERVEIDHTPLDLILVDDELLLPLGRPYLTALMDSYSGCIVGFYIGYREPSYDSVRRALSCAYLPKHWVKERFPSIKKEWPCEGKIGMLVVDNAAEFWSSSLDDACAGIVQNVDYNQVARPWLKPMIERFFSTVNKKLLISIPGKTFSSIQELKDYKPEKDAVMRFSTFMELFHKWLIDEYHYRPDTRETKIPIVQWCKGTSLVSPPTYEANEAERLLIELAKVNERSVLHDGIHIHKLRYVSDELTEYRKRKSPETGAKHLKVKVKTIHTSIAYIFVFLQSEQRYIKVPCVDQEYASGLSLLQHQTNQRFVRSYVRSSVDTEHLAECKVYLHERIRKEAEALSQKVKRKNPKIGGMKKMAKYHNIGSDSGNGSITAAQAIQTQTLLANNTKPTDIEDLDWENFELEDGAY

SEQ ID NO: 171 - protein, 879 aa, synthetic construct
MKSRVIGPSTHKSIFKFASPKMGKMVKVESSLEYDACFHFEYSPSITSFIAQPCGVDYQLNGRTQTFYPDFLVEDKEFGKRFFEIKPSSKVRKPEFRVKFALRREAALSQSIPLIVVTEKQICLNPILNNLKLLHRYAGNYSLTPLHFWVLDAVKSLGRITVRDLVDESDCAPGDVFASALTWISRGHLQADISDNELGVNSLVWCGSGPKKKRKVGSGYPYDVPDYAYPYDVPDYAYPYDVPDYAGSGASSRHTLGLFDDEYDSLSAESIESFSKETSDLDNDHLSPDFDSYSKEQQREALRRYALIQWVDKRLKGGWTEKKLSPLLEQATIEFDFTLPNWRTLSRWYSSYINSGHSLEALLPKHHKKGGTGARKMEDGFFFEKAIEEYYLTRERPTIADCYELYKSWIVLENSKLISGKLKPVCQRTFYNRINKLSPYLVALRRFGKPYADRHFRTVKQLKKPSNVLERVEIDHTPLDLILVDDELLLPLGRPYLTALMDSYSGCIVGFYIGYREPSYDSVRRALSCAYLPKHWVKERFPSIKKEWPCEGKIGMLVVDNAAEFWSSSLDDACAGIVQNVDYNQVARPWLKPMIERFFSTVNKKLLISIPGKTFSSIQELKDYKPEKDAVMRFSTFMELFHKWLIDEYHYRPDTRETKIPIVQWCKGTSLVSPPTYEANEAERLLIELAKVNERSVLHDGIHIHKLRYVSDELTEYRKRKSPETGAKHLKVKVKTIHTSIAYIFVFLQSEQRYIKVPCVDQEYASGLSLLQHQTNQRFVRSYVRSSVDTEHLAECKVYLHERIRKEAEALSQKVKRKNPKIGGMKKMAKYHNIGSDSGNGSITAAQAIQTQTLLANNTKPTDIEDLDWENFELEDGAY

SEQ ID NO: 172 - protein, 347 aa, Agarivorans gilvus
MAQLLEMQQSQFDSFLDCFIEHPTVTTIYEIFDRLRFHFHSHQRISAGAAADVPCMLLTGDSGSGKSSLVRHYRQQAQASPDSQLNVTPVLVTRIPDTPSLDLTILEMLSTLGHFGTSFRYKASNSLSLTASLLKALAYKKTELIIINEVQELFEFKSLKECTAISNRLKYISEESGIPFVLVGMPWADKITDDPQWDSRLIHKQFLPYFNLSSKSDLKEFSRLINGFCLRMGFDVPPKLNDKHTIRALFSACSGQMRSLKSLLSEALFLALKDRALTIELKHLEEAFIFQKPGVSNPFKMAFEEIPVPKVKEYSKLNHAASTLDEQIIRTQFVDGLPISQLLKKNS

SEQ ID NO: 173 - protein, 362 aa, synthetic construct
MAQLLEMQQSQFDSFLDCFIEHPTVTTIYEIFDRLRFHFHSHQRISAGAAADVPCMLLTGDSGSGKSSLVRHYRQQAQASPDSQLNVTPVLVTRIPDTPSLDLTILEMLSTLGHFGTSFRYKASNSLSLTASLLKALAYKKTELIIINEVQELFEFKSLKECTAISNRLKYISEESGIPFVLVGMPWADKITDDPQWDSRLIHKQFLPYFNLSSKSDLKEFSRLINGFCLRMGFDVPPKLNDKHTIRALFSACSGQMRSLKSLLSEALFLALKDRALTIELKHLEEAFIFQKPGVSNPFKMAFEEIPVPKVKEYSGSGPAAKKKKLDGSGKLNHAASTLDEQIIRTQFVDGLPISQLLKKNS

SEQ ID NO: 174 - protein, 406 aa, Agarivorans gilvus
MFLIPEDYHEDESLESYLLRISQANGFESYALLSGAVKEFLRQHDAEAYGAFPLELSLVNIYHAKLSSSFRVRAIRLMEELIGLSTWQLNRLALKHTAQTIVGSYTILVRQKEFLPRAFLRQGSVPVCPQCLSVQPYIRQNWHFLPCTACNLHQTKLLCHCPECGEALNYQKTELIEYCQCGYDLRSVRTNVASKAECQLSAIFDKSREASNNPLLVCRHTSIRTGALLWYCLWRNVELDELVVDKNHAQDCIGFFERWPDEINKELAAIAEAAEQRLVEPFNKTAFSAVFGGLLNRSRVAPLSMSSEDFIHQSVIQFLVHLVMDNPKSKQPNIADLQLTVPEVAALLNCSREQVYRYYEEGMLELTFRLRLHNTLSLNKPAFFLRQAVELAISLTSGSGDPLPAW

SEQ ID NO: 175 - protein, 449 aa, synthetic construct
MPKKKRKVGSGEQKLISEEDLEQKLISEEDLEQKLISEEDLGSGFLIPEDYHEDESLESYLLRISQANGFESYALLSGAVKEFLRQHDAEAYGAFPLELSLVNIYHAKLSSSFRVRAIRLMEELIGLSTWQLNRLALKHTAQTIVGSYTILVRQKEFLPRAFLRQGSVPVCPQCLSVQPYIRQNWHFLPCTACNLHQTKLLCHCPECGEALNYQKTELIEYCQCGYDLRSVRTNVASKAECQLSAIFDKSREASNNPLLVCRHTSIRTGALLWYCLWRNVELDELVVDKNHAQDCIGFFERWPDEINKELAAIAEAAEQRLVEPFNKTAFSAVFGGLLNRSRVAPLSMSSEDFIHQSVIQFLVHLVMDNPKSKQPNIADLQLTVPEVAALLNCSREQVYRYYEEGMLELTFRLRLHNTLSLNKPAFFLRQAVELAISLTSGSGDPLPAW

SEQ ID NO: 176 - protein, 207 aa, Vibrio cholerae; identical to SEQ ID NO: 15

SEQ ID NO: 177 - protein, 621 aa, Vibrio cholerae; identical to SEQ ID NO: 16

SEQ ID NO: 178 - protein, 870 aa, synthetic construct
MFDQTKKSSHVHNICKFMSLKNDAVVRTLSILEFDFCFHLEYNPNIKSFTSQPFGFHYLFNNRKCRYTPDFLAIGHNEQSTFFEVKHSSQIPKPDFRERFEEKQRVALSEFNRRLVLVTEKQIRMGPTLDNFKLLHRYSGLRTVTEFQKRVLAFIQRKQMVKLQEVSLYFGLSEQDTLISTLPWISSGHVKTDLNTIGFGLETCVWCGSGPKKKRKVGSGYPYDVPDYAYPYDVPDYAYPYDVPDYAGSGPPDSNSIFGFFDEFEASEEESQLLPKELILEPVEISSTIDSLPAKIQEEVLRRIKVITFVEKRLKGGWTEKNLNPILSLVESELQLTPPSWRTVATWKKSYAEAGREASALIPKHTFKGNRQKEMDSQSLIDEAIQNVYLTRERLSVAEAYRYYKSRVIQMNRGIVEGKIKPIAERSFYNRINELPPYEVAIARFGKRYADREYRSVGQQVVATKPMEFVEIDHTPVPVILIDDELDIPLGRPYLTMLYDRFSKCIVGCSINFREPSFDSVRKALLNSLLDKSWLKAKYPSIENEWPCHGKIDCLVVDNGAEFWSQSLEDSLRPLVSDIQYSQAAKPWRKSGIEKLFDQMNKGLVNALPGKTFTNPTQLQDYNPKKDAVVRVSVFLELLHKWIVDYYHMAPDSREREIPYHKWHQSKWTPSYYDGAEKEQLRVELGLLRHRTIGVAGIRLHNLRYQSAELIEYRKYCTPNNGKQLFVKTKTDPSDISYIHVYLESEKKYIKVPAVDNSGYTNGLSLFEHQRIQKVRRLNTKDLADDEALADTFLYMKKRIHEETDRFRRVKSSKPNLPKTGNTSRLAKFNDVGSEGPNSINVTPVRLKSEVVSDASEYLDDDDFEDIEGY

SEQ ID NO: 179 - protein, 353 aa, Vibrio cholerae
LNLTPKQLEQLKSFETCFIEYPAITEIYSIFDQLRFNHSLGGEPESFLLTGEAGSGKTALINNYLSRFQSGSTWGKQPVLSTRVPSRINEQNTLTQFLVDLDCKSGGRGIRRRNEIALGEAVVKQLKRKSVELIIVNEIQELVEFSTAEQRQVIANTFKYMSEEARVSFVLVGMPYADVIATEPQWNSRLSWRRKIDYFKLLKANSHSSKTASYGFDLEQKKHFARFVVGLSSRMGFDEPPVLTKNELLYPLFAMCRGECRALKHFLKDALLTSFNDNADTIDKAILSRTFAFKFPYLDNPFDRPLEQLSLHQIDSGSAYHLNAITTEDKIVAPRFTDAIPLSMLLSKNGLKA

SEQ ID NO: 180 - protein, 380 aa, synthetic construct
MTMILKILKGISLNLTPKQLEQLKSFETCFIEYPAITEIYSIFDQLRFNHSLGGEPESFLLTGEAGSGKTALINNYLSRFQSGSTWGKQPVLSTRVPSRINEQNTLTQFLVDLDCKSGGRGIRRRNEIALGEAVVKQLKRKSVELIIVNEIQELVEFSTAEQRQVIANTFKYMSEEARVSFVLVGMPYADVIATEPQWNSRLSWRRKIDYFKLLKANSHSSKTASYGFDLEQKKHFARFVVGLSSRMGFDEPPVLTKNELLYPLFAMCRGECRALKHFLKDALLTSFNDNADTIDKAILSRTFAFKFPYLDNPFDRPLEQLSLHQIDSGSGSGPAAKKKKLDGSGAYHLNAITTEDKIVAPRFTDAIPLSMLLSKNGLKA

SEQ ID NO: 181 - protein, 403 aa, Vibrio cholerae
VKTDIQHYSDESLESFLLRLSQEQGYERFSHFAEDIWFDTMEQHEAIAGAFPLELNRINIYHAQTTSQMRVRVLIHLENQLKLNNFGVLRLALSHSKAQFSPEYKAVHRLGSDYPFVFLGKRFTPICPLCISEAPYIRQQWQFLSQQACERHGCKLVHHCPECQSRLEYQTTESISQCECGFELRNSPVEDAPVAALLVARWLSGNDSKPLGLLKAEMTLSERYGFLLWYVNRYGDIENISFESFVEYCSCWPRVLKEELDELVNKADLIRIKDWKKTFFNEVFGALLKDCRQLPSRQLECNSVLTQVLAYFTKLMAAIPSSSKGNVGDVLLSPLEASTLLSCTTDEVYRLYEFGEIKAAIRPRMHTKIASHESAFTLRSVIETKLTRMCSENDGLSVYLPEW

SEQ ID NO: 182 - protein, 446 aa, synthetic construct; identical to SEQ ID NO: 21

SEQ ID NO: 183 - protein, 208 aa, Vibrio hyugaensis
MYIRNLRKPSPNKNIFKFSSLKNRDAVMCEGSLEKDCCYHFEYDPDVVRYESQPEGFYYDFNGKKRPYTPDFLVTYHDGTFEYVEVKPHTKTLSKTFKQEFSARKEAANRRGVSLVLVTDKQIRDGYFLKNTELVHRYSGCIAGDELAIKVYSYLIAQNTMKISDLADSIGESVGRVFASVLRLIAVGKAGVDLDIAQLSESTTVSVR

SEQ ID NO: 184 - protein, 638 aa, Vibrio hyugaensis
MVGRFHDEFEPEYDEDSDLKHEFLPAAQTESLKYSRLQSTQIIERDLSSYPEEQKNKALERYKLLCLVANELNGGWTSKNLTPLIEKHFDKTCLPKKPSYKSLQRWHNSFVDSDGSFTSLVDKNHLKGNRDARVVGDEKYYDEALKMFLDARRQSIRAAHAFYCDRITVANEAIVAGRIPKVSYEAFKKRIRKEEPYSVVLARHGKYYADKLFNYYQSVEMPTRILERVEMDHTPLDLILLHDELMVPLGRAHLTLLVDVFSGCIIGFHLGFKAPSYVSASRAVIHATKSKSYISEMPISFNNEWLCEGKIENLVVDNGAEFWSKSWEDACLEVGINVVYNKVRKPWLKPFIERKFGEIVQGIVGWVPGKTFSNVLEKEDYKPEKDAVMRFSTFVEEFHRWIVDVHNANADSRYKRIPNLYWKQSYDALPPLKLLPEHEQAFRVVMGILQYRKLTDKGIKFMHLEYDCVALSDYRKTYPQTNESSKKKIKVDPDDLSAIYVYLDELQGYVKVPSKDPMGYTVRLSVCEHEKILAAHRTYIKGEMDVLSLAKARLALHDRIESEQADLMQLTHTERKRKAKSTKKVAEISSVNSDTPHSKLSDRTPKPNKKVAESEKSSDTTPLESFRAKWDERRNLRK

SEQ ID NO: 185 - protein, 888 aa, synthetic construct
MYIRNLRKPSPNKNIFKFSSLKNRDAVMCEGSLEKDCCYHFEYDPDVVRYESQPEGFYYDFNGKKRPYTPDFLVTYHDGTFEYVEVKPHTKTLSKTFKQEFSARKEAANRRGVSLVLVTDKQIRDGYFLKNTELVHRYSGCIAGDELAIKVYSYLIAQNTMKISDLADSIGESVGRVFASVLRLIAVGKAGVDLDIAQLSESTTVSVRGSGPKKKRKVGSGYPYDVPDYAYPYDVPDYAYPYDVPDYAGSGVGRFHDEFEPEYDEDSDLKHEFLPAAQTESLKYSRLQSTQIIERDLSSYPEEQKNKALERYKLLCLVANELNGGWTSKNLTPLIEKHFDKTCLPKKPSYKSLQRWHNSFVDSDGSFTSLVDKNHLKGNRDARVVGDEKYYDEALKMFLDARRQSIRAAHAFYCDRITVANEAIVAGRIPKVSYEAFKKRIRKEEPYSVVLARHGKYYADKLFNYYQSVEMPTRILERVEMDHTPLDLILLHDELMVPLGRAHLTLLVDVFSGCIIGFHLGFKAPSYVSASRAVIHATKSKSYISEMPISFNNEWLCEGKIENLVVDNGAEFWSKSWEDACLEVGINVVYNKVRKPWLKPFIERKFGEIVQGIVGWVPGKTFSNVLEKEDYKPEKDAVMRFSTFVEEFHRWIVDVHNANADSRYKRIPNLYWKQSYDALPPLKLLPEHEQAFRVVMGILQYRKLTDKGIKFMHLEYDCVALSDYRKTYPQTNESSKKKIKVDPDDLSAIYVYLDELQGYVKVPSKDPMGYTVRLSVCEHEKILAAHRTYIKGEMDVLSLAKARLALHDRIESEQADLMQLTHTERKRKAKSTKKVAEISSVNSDTPHSKLSDRTPKPNKKVAESEKSSDTTPLESFRAKWDERRNLRK

SEQ ID NO: 186 - protein, 338 aa, Vibrio hyugaensis
MGRAQKSKEIVVTAARRNLNRDEVLANYHDSFSIYPEVEKVLSGLEWIIKRRKFGTFAPSMLLTAGTGAGKTATINHFIEKNLSRNEVLITRVRPSLLETLLWMAKELGAYRNSRAKPSEIGLTDCVIETSKRVGLKLLVIEECQELFERTSHNQRQDIRDRLKMISDECHLPIVFVGLHSAGLILEDSQWNRRIMVRRTLPYIKITDESAIDNYLDVLQALEKTVPLPFKVPLTDVDFAMRLLSASKGILGEIKELIAAALDVALAKNKDYIGEEDFAAVYEKINDPNDINPFTVQIDALTIEQIASYENYVTDAETGELRFVKQVFSKLSIQQLIG

SEQ ID NO: 187 - protein, 353 aa, synthetic construct
MGRAQKSKEIVVTAARRNLNRDEVLANYHDSFSIYPEVEKVLSGLEWIIKRRKFGTFAPSMLLTAGTGAGKTATINHFIEKNLSRNEVLITRVRPSLLETLLWMAKELGAYRNSRAKPSEIGLTDCVIETSKRVGLKLLVIEECQELFERTSHNQRQDIRDRLKMISDECHLPIVFVGLHSAGLILEDSQWNRRIMVRRTLPYIKITDESAIDNYLDVLQALEKTVPLPFKVPLTDVDFAMRLLSASKGILGEIKELIAAALDVALAKNKDYIGEEDFAAVYEKINDPNDINPFTVQIDALTIEQIASYEGSGPAAKKKKLDGSGNYVTDAETGELRFVKQVFSKLSIQQLIG

SEQ ID NO: 188 - protein, 403 aa, Vibrio hyugaensis
VETDIQLYPDESLESFLLRLSQEQGYERFSHFAEDIWFDTLNQHEAIAGAFPLELNRVNIYHAQTTSQMRVRVLIHLENQLKLNNFGVLRLALSHSKAQFSSQYKAVHRFGSDYPYAFLRKRFTPICPLCVDEAPYIRQQWQLISHQACEHHGCKLVHHCPECKSRLEYQSTESISQCECGYELRNSPVEDAPEAEVLVARWLSGNDSKPLGLLTGEMTLSERYGFLLWYINRYGDIDDLSFESFIEYCCAWPTALWQDLDALKEKAELVRVKDWKKMFFNEAFDTLLKGCRQLPSRQLSHNTVLTQVLAYFTQLMATVPSSAKGNIGDALLSPLEASTLLSCTTDEVYRLYEFGEIKAAIRPRMHTKIASHESAFTLRSVIETKLTRMSSESDGLSVYLPEW

SEQ ID NO: 189 - protein, 446 aa, synthetic construct
MPKKKRKVGSGEQKLISEEDLEQKLISEEDLEQKLISEEDLGSGETDIQLYPDESLESFLLRLSQEQGYERFSHFAEDIWFDTLNQHEAIAGAFPLELNRVNIYHAQTTSQMRVRVLIHLENQLKLNNFGVLRLALSHSKAQFSSQYKAVHRFGSDYPYAFLRKRFTPICPLCVDEAPYIRQQWQLISHQACEHHGCKLVHHCPECKSRLEYQSTESISQCECGYELRNSPVEDAPEAEVLVARWLSGNDSKPLGLLTGEMTLSERYGFLLWYINRYGDIDDLSFESFIEYCCAWPTALWQDLDALKEKAELVRVKDWKKMFFNEAFDTLLKGCRQLPSRQLSHNTVLTQVLAYFTQLMATVPSSAKGNIGDALLSPLEASTLLSCTTDEVYRLYEFGEIKAAIRPRMHTKIASHESAFTLRSVIETKLTRMSSESDGLSVYLPEW

SEQ ID NO: 190 - protein, 207 aa, Vibrio crassostreae
MYDQTKKSSAVHNICKFMSLKNDSVVRTMSMLEYDFCFHAEYNPQIVRYESQPHGFEYYFNGRYCRYTPDFQLFDSIDTPSLIEVKHSSQILKPDFRARFKEKQLVAQAEYGKKLILVTEKQIRTGFLLSNLKLLHGYSGIRTITDIQKHVLQFVQANRSVTLHHLAHQLKISPDETLTAALCWLSSGEIQTDFNQKKFDLENSVWC

SEQ ID NO: 191 - protein, 620 aa, Vibrio crassostreae
MSDDLFGFSDEFNSFDNDVADDKTLSTEFLAEYENLELAFGDLPNKETALFRLDLIRYLERRVKGGWTPKNLDKLLEEYALLKKTSVPSSRTIADWKKLYYESGKDLASLIPGHSKKGNRKLKNDSSDLVTEAIQTKFLTKERVSVNTAYEYYKYRVIEENRQLDQVKIAPISQRTFYNRVNALPPYEVALARYGKRYADNKFKTVGSIIPATRPMEYVEIDHTTAPVILLDDDLELPLGRPHLTILYDRYSTCIVGLSVNYRDPSYETVRAAFLNSVLKKDWIKEKYPSIESDWPCYGKITNLIVDNGAEFWSDSLESALKPLVTDIQYNQRGKPWRKAGVEKSFDTFYKKLFSRFPGKTFTNPTQLKDYNPKQDAVINVSDFLELLHKWLIDVYHKKADTRYKRVPYQKWTESQGTIIFCEGPEAEQLKIELGAVNHRTIRRGAIELYSLKYQSDELEEYGKQYSSRARKSAYVKIKTDPNDISSIYVYLEEEKRYIKVPAVDHTGYTKGRSLYEHQRINSLRRLKVRLGEQDESLADASLYLDRAMDEAIERMSRSKSKKSALPKTTHASKIAKQRGVGSEGPSTIVTTSPKPIIEVPKEVIDMGTTSDDLSDIEGY

SEQ ID NO: 192 - protein, 869 aa, synthetic construct
MYDQTKKSSAVHNICKFMSLKNDSVVRTMSMLEYDFCFHAEYNPQIVRYESQPHGFEYYFNGRYCRYTPDFQLFDSIDTPSLIEVKHSSQILKPDFRARFKEKQLVAQAEYGKKLILVTEKQIRTGFLLSNLKLLHGYSGIRTITDIQKHVLQFVQANRSVTLHHLAHQLKISPDETLTAALCWLSSGEIQTDFNQKKFDLENSVWCGSGPKKKRKVGSGYPYDVPDYAYPYDVPDYAYPYDVPDYAGSGSDDLFGFSDEFNSFDNDVADDKTLSTEFLAEYENLELAFGDLPNKETALFRLDLIRYLERRVKGGWTPKNLDKLLEEYALLKKTSVPSSRTIADWKKLYYESGKDLASLIPGHSKKGNRKLKNDSSDLVTEAIQTKFLTKERVSVNTAYEYYKYRVIEENRQLDQVKIAPISQRTFYNRVNALPPYEVALARYGKRYADNKFKTVGSIIPATRPMEYVEIDHTTAPVILLDDDLELPLGRPHLTILYDRYSTCIVGLSVNYRDPSYETVRAAFLNSVLKKDWIKEKYPSIESDWPCYGKITNLIVDNGAEFWSDSLESALKPLVTDIQYNQRGKPWRKAGVEKSFDTFYKKLFSRFPGKTFTNPTQLKDYNPKQDAVINVSDFLELLHKWLIDVYHKKADTRYKRVPYQKWTESQGTIIFCEGPEAEQLKIELGAVNHRTIRRGAIELYSLKYQSDELEEYGKQYSSRARKSAYVKIKTDPNDISSIYVYLEEEKRYIKVPAVDHTGYTKGRSLYEHQRINSLRRLKVRLGEQDESLADASLYLDRAMDEAIERMSRSKSKKSALPKTTHASKIAKQRGVGSEGPSTIVTTSPKPIIEVPKEVIDMGTTSDDLSDIEGY

SEQ ID NO: 193 - protein, 356 aa, Vibrio crassostreae
LVNILSELQIEQYTSFRECFLEYPQLTEIYNVFDRMVLNSSLGGEQESLLLTGDTGVGKTAMIDNYVARFAIKGSRWAEMPVLKTRIPSKVREQNTLERLLIDLDSRASSRRRRPYKEGALEQGVIKSLIEKKVKLVIVNEVQELMEFKDANERQTIANTFKMISEEAQVSFVLVGMPYATMLAEEDQWNSRLGWKRHLSYFHLSKLSEADKKGYIPDAEGKRHFASFVAGLAGRMGFEKRPNLTGDEILLPLFSVCRGECRVLKHFLADALLNALQSSKDTIDKPLLSACFDTKYPYAKQNPFECKLTELKLVELKTETSYNKGAQFKEDRLIGRSFTDLLPVHMLLSKTPLKAQ

SEQ ID NO: 194 - protein, 370 aa, synthetic construct
MNILSELQIEQYTSFRECFLEYPQLTEIYNVFDRMVLNSSLGGEQESLLLTGDTGVGKTAMIDNYVARFAIKGSRWAEMPVLKTRIPSKVREQNTLERLLIDLDSRASSRRRRPYKEGALEQGVIKSLIEKKVKLVIVNEVQELMEFKDANERQTIANTFKMISEEAQVSFVLVGMPYATMLAEEDQWNSRLGWKRHLSYFHLSKLSEADKKGYIPDAEGKRHFASFVAGLAGRMGFEKRPNLTGDEILLPLFSVCRGECRVLKHFLADALLNALQSSKDTIDKPLLSACFDTKYPYAKQNPFECKLTELKLVELKTETGSGPAAKKKKLDGSGSYNKGAQFKEDRLIGRSFTDLLPVHMLLSKTPLKAQ

SEQ ID NO: 195 - protein, 363 aa, Vibrio crassostreae
MDQHEAIAGAFPLDLNLVNIYHAQTTSQMRVRVLIHLENQLKLNNFGVLRLALSHSKAQFSPQYKAVHRFGSDYPYAFLRKRFTPICPLCIDEAPYIRQQWQFISHQACEHHGCKLIHHCPECKLRLEYQSTESSSQCECGFELRNSPVEGAPEVEVLVAQWLSGNDSKPLGLLKGEMTLSERYGFLLWYVNRHGDIDDLSFESFIEYCGSWPTALWQDLDALKEKAELIRVKDWKKMFFNEAFGALLKDCRQLPSRQLSHNIVLTRVLAYFAKLMATVPSSAKGNIGDVLLSPLEASTLLSCTTDEVYRLYEFGEIKAAIRPRMHTKIASHESAFTLRSVIETKLIRMSSESDGLSVYLPEW

SEQ ID NO: 196 - protein, 406 aa, synthetic construct
MPKKKRKVGSGEQKLISEEDLEQKLISEEDLEQKLISEEDLGSGDQHEAIAGAFPLDLNLVNIYHAQTTSQMRVRVLIHLENQLKLNNFGVLRLALSHSKAQFSPQYKAVHRFGSDYPYAFLRKRFTPICPLCIDEAPYIRQQWQFISHQACEHHGCKLIHHCPECKLRLEYQSTESSSQCECGFELRNSPVEGAPEVEVLVAQWLSGNDSKPLGLLKGEMTLSERYGFLLWYVNRHGDIDDLSFESFIEYCGSWPTALWQDLDALKEKAELIRVKDWKKMFFNEAFGALLKDCRQLPSRQLSHNIVLTRVLAYFAKLMATVPSSAKGNIGDVLLSPLEASTLLSCTTDEVYRLYEFGEIKAAIRPRMHTKIASHESAFTLRSVIETKLIRMSSESDGLSVYLPEW

SEQ ID NO: 197 - protein, 207 aa, Aeromonas salmonicida
MYRRHLKHSRVKNLFKFVSAKMNTVFTVESSLEFDTCFHLEYSPAVKAFEAQPEGFYYTFEGRDCPYTPDFRVLNENGSVGYLEVKPSAKVLESDFLQRFPFKQQRATELSCPLKLITERQIRIDPILGNLKLLHRYSGFQSFTPLHMQLLGLVKDFGRVSLARLSGSTGAPPGEVLATVLSLIARGLIHSDLAEHEMGFSTIVWMR

SEQ ID NO: 198 - protein, 629 aa, Aeromonas salmonicida
MCAQPTTEVPSDLFEDEFTHPHPPESPNLAATTPTVLSATVDSFPADLKAQALHRLDYIRWIEDNLAGGWTEKNLAPLLVEAAKVLPPPAPNWRTLARWQKNYNQHGRKLMALIPKHQAKGNVKSRLPSSDEVFFEQAVHCFLVGEQPSIASVYQYYTDIICIENLNVVENPIKAISYTAFFNRLKKLPAYQVIKSRKGSYMADVEFMAISSHIPPSCVMERVEIDHTPLDLILLDDDLLVPLGRPCLTLLIDSYSHCVVGFNLSFNQPGYESVRNALLNSIPPKNYVKDKYPSVEHEWPCYGKPATLVVDNGVEFWSKSLEQSCRELNINTQYNPVRKPWLKPMVERMFGTINRKLLESIPGKTFSNLLERGEYDPQKDAVMRFSTFLEIFHRWIIDVYHYEPDSRRRYIPIQSWQYGCNKLPPAPVVGDDLAKLEVILSISLQCTHRRGGIQRFHLRYDSDELASYRMNYPDKTHGKRKVLVKLNPRDISYVFVFIKEAGSFIRVPCIDPEGYTKGLSLQEHQINMKLHRDFIDTQMDVVSLAKARTYINSRIQSELSEVRQTLKKRNTKGINKIARYRDIGSQTTTGLLSGPQLSESKDDVPIQPKTTPPQLEDDWDSFTSGLEPY

SEQ ID NO: 199 - protein, 878 aa, synthetic construct
MYRRHLKHSRVKNLFKFVSAKMNTVFTVESSLEFDTCFHLEYSPAVKAFEAQPEGFYYTFEGRDCPYTPDFRVLNENGSVGYLEVKPSAKVLESDFLQRFPFKQQRATELSCPLKLITERQIRIDPILGNLKLLHRYSGFQSFTPLHMQLLGLVKDFGRVSLARLSGSTGAPPGEVLATVLSLIARGLIHSDLAEHEMGFSTIVWMRGSGPKKKRKVGSGYPYDVPDYAYPYDVPDYAYPYDVPDYAGSGCAQPTTEVPSDLFEDEFTHPHPPESPNLAATTPTVLSATVDSFPADLKAQALHRLDYIRWIEDNLAGGWTEKNLAPLLVEAAKVLPPPAPNWRTLARWQKNYNQHGRKLMALIPKHQAKGNVKSRLPSSDEVFFEQAVHCFLVGEQPSIASVYQYYTDIICIENLNVVENPIKAISYTAFFNRLKKLPAYQVIKSRKGSYMADVEFMAISSHIPPSCVMERVEIDHTPLDLILLDDDLLVPLGRPCLTLLIDSYSHCVVGFNLSFNQPGYESVRNALLNSIPPKNYVKDKYPSVEHEWPCYGKPATLVVDNGVEFWSKSLEQSCRELNINTQYNPVRKPWLKPMVERMFGTINRKLLESIPGKTFSNLLERGEYDPQKDAVMRFSTFLEIFHRWIIDVYHYEPDSRRRYIPIQSWQYGCNKLPPAPVVGDDLAKLEVILSISLQCTHRRGGIQRFHLRYDSDELASYRMNYPDKTHGKRKVLVKLNPRDISYVFVFIKEAGSFIRVPCIDPEGYTKGLSLQEHQINMKLHRDFIDTQMDVVSLAKARTYINSRIQSELSEVRQTLKKRNTKGINKIARYRDIGSQTTTGLLSGPQLSESKDDVPIQPKTTPPQLEDDWDSFTSGLEPY

SEQ ID NO: 200 - protein, 335 aa, Aeromonas salmonicida
MELSSTDADKLKSFIECYVETPLLRIIQDDFDRLRYDKQFAGEPICMLLTGDSGTGKSSLIRHYMAQFPEQHGHGFVRKPLLVSRIPSKPTLESTMVELLKDLGQWGSEYRLHRSSAESLTEALIKCLTRCETELIIIDEFQELIENKTREKRNQIANRLKYISETAKIPIVLVGMPWAAKIAEEPQWASRLMVQRTIPFFKLSEDAESFVRFVMGLARRMPFATPPKLEAKHTIFALFAFSYGCVRRLKHLLDESVKQALAAHSETLLHEHIAVAFGLFYPDQENPFLQSIDEIKACEVTQYSRYEINESGTEEVLNPLKFTDKIPISQLLKKR

SEQ ID NO: 201 - protein, 350 aa, synthetic construct
MELSSTDADKLKSFIECYVETPLLRIIQDDFDRLRYDKQFAGEPICMLLTGDSGTGKSSLIRHYMAQFPEQHGHGFVRKPLLVSRIPSKPTLESTMVELLKDLGQWGSEYRLHRSSAESLTEALIKCLTRCETELIIIDEFQELIENKTREKRNQIANRLKYISETAKIPIVLVGMPWAAKIAEEPQWASRLMVQRTIPFFKLSEDAESFVRFVMGLARRMPFATPPKLEAKHTIFALFAFSYGCVRRLKHLLDESVKQALAAHSETLLHEHIAVAFGLFYPDQENPFLQSIDEIKACEVTQYSGSGPAAKKKKLDGSGRYEINESGTEEVLNPLKFTDKIPISQLLKKR

SEQ ID NO: 202 - protein, 410 aa, Aeromonas salmonicida
MQLLVRPAPFSDESLESYLLRLSQENGFERYALLSGAMRDALLQQDHQAAGAFPLELARVNVFHANRSSSLRVRALHLIEQLTDLAPHSLLQLALIRSAMPIGAGHACVQRGGVDIPLRLVRTRQIPVCPVCLSESAYIRQHWHYAPYVACHLHGHELLSVCPSCGKALDYQCNESFTHCRCGFDLRHSITPPASNQAIQISALICGARWESTNPLLICPHPSQLFGAIFWYWCRYHAEAAGQPASHSLVQTIDYFAAWPANFHAELDQWAQRGLLRQTRLLNETPFGEVFGAVLSDCRQLPFQDLGANFILRALSDYLTALVVNHPKTRQPNLGDILLSASDAAALLSTSVEQVFRLQQEGYLTLAYRLRRHAGLTPYDPMFHLRQVIEYRLAHGAMYPPAFYSFLPAW

SEQ ID NO: 203 - protein, 453 aa, synthetic construct
MPKKKRKVGSGEQKLISEEDLEQKLISEEDLEQKLISEEDLGSGQLLVRPAPFSDESLESYLLRLSQENGFERYALLSGAMRDALLQQDHQAAGAFPLELARVNVFHANRSSSLRVRALHLIEQLTDLAPHSLLQLALIRSAMPIGAGHACVQRGGVDIPLRLVRTRQIPVCPVCLSESAYIRQHWHYAPYVACHLHGHELLSVCPSCGKALDYQCNESFTHCRCGFDLRHSITPPASNQAIQISALICGARWESTNPLLICPHPSQLFGAIFWYWCRYHAEAAGQPASHSLVQTIDYFAAWPANFHAELDQWAQRGLLRQTRLLNETPFGEVFGAVLSDCRQLPFQDLGANFILRALSDYLTALVVNHPKTRQPNLGDILLSASDAAALLSTSVEQVFRLQQEGYLTLAYRLRRHAGLTPYDPMFHLRQVIEYRLAHGAMYPPAFYSFLPAW

SEQ ID NO: 204 - protein, 209 aa, Klebsiella oxytoca
MYRRHLHHSRVKNLFKFASVRMGIVLTLESSLEFDTCFQLEYSPAVKTYISQPEGFYYEFEGKSYPYTPDFLVKDQNDQEFLLEVKPSSQIDDIDFLQRFPAKQKKAKELASPLILITEKQIRSTPLLDNLKLVHRYAGFHSIMPSCNEIMELLREQKEVAIFNLCESIDIPQGEMYSSILLLLSRGLISGNLMESEFGLVTLLKYAQQ

SEQ ID NO: 205 - protein, 622 aa, Klebsiella oxytoca
NSLFICSFPFEDEFTLSQENEVKMSTDESSDIILPATLDCYSEILKEESVRRLNYIQWVEKRIIGGWTEKNITPLINEVAQTLRPPAPHWRQLVRWHKKYLQHRRQITALVPNHKNKGNKTQRVSSREEIFIENAILKFQSKERPSISSMYCFYCDSVRIFNLSNSTERIKTVSLNTFYRRIKKLSVYQVMNARDGRVAANMEFQAIDSFLPTSRVLERVEIDHTPLDLILLDDELLLPLGRPSLTLLIDVYSHCAVGFNLCFTQPGYESVRCALLHSLVRKDYVQEQYPCIENSWISYGKPETLVVDNGAEFWSSSLEHACLELGINTQYNPVRKPWLKPLIERMFGTINRKFLESIPGKTFSNILDKADYNPQKDAVMRFSVFLEIFHHWLLDVYHYEPDSRYRYVPALAWKYGCKVYPPATIEKNELKKLEIILSISLRRLHRRGGIHLHHLRYDSKELSALRMQYSLEEKGKKKVLVKLNPADMSYIYVYIDKIKSYIRVPCVDPCKYTQNLSLQQHLINLRFHRDFINENINLDSLSKARIYISERIQGEIDNVRQYAKRSSKKGMKKIASHQGVTSQNKKTIASDTIHFPAQKGKNRDTHTLPDDWDDFTSDLEPF

SEQ ID NO: 206 - protein, 874 aa, synthetic construct
MYRRHLHHSRVKNLFKFASVRMGIVLTLESSLEFDTCFQLEYSPAVKTYISQPEGFYYEFEGKSYPYTPDFLVKDQNDQEFLLEVKPSSQIDDIDFLQRFPAKQKKAKELASPLILITEKQIRSTPLLDNLKLVHRYAGFHSIMPSCNEIMELLREQKEVAIFNLCESIDIPQGEMYSSILLLLSRGLISGNLMESEFGLVTLLKYAQQGSGPKKKRKVGSGYPYDVPDYAYPYDVPDYAYPYDVPDYAGSGNSLFICSFPFEDEFTLSQENEVKMSTDESSDIILPATLDCYSEILKEESVRRLNYIQWVEKRIIGGWTEKNITPLINEVAQTLRPPAPHWRQLVRWHKKYLQHRRQITALVPNHKNKGNKTQRVSSREEIFIENAILKFQSKERPSISSMYCFYCDSVRIFNLSNSTERIKTVSLNTFYRRIKKLSVYQVMNARDGRVAANMEFQAIDSFLPTSRVLERVEIDHTPLDLILLDDELLLPLGRPSLTLLIDVYSHCAVGFNLCFTQPGYESVRCALLHSLVRKDYVQEQYPCIENSWISYGKPETLVVDNGAEFWSSSLEHACLELGINTQYNPVRKPWLKPLIERMFGTINRKFLESIPGKTFSNILDKADYNPQKDAVMRFSVFLEIFHHWLLDVYHYEPDSRYRYVPALAWKYGCKVYPPATIEKNELKKLEIILSISLRRLHRRGGIHLHHLRYDSKELSALRMQYSLEEKGKKKVLVKLNPADMSYIYVYIDKIKSYIRVPCVDPCKYTQNLSLQQHLINLRFHRDFINENINLDSLSKARIYISERIQGEIDNVRQYAKRSSKKGMKKIASHQGVTSQNKKTIASDTIHFPAQKGKNRDTHTLPDDWDDFTSDLEPF

SEQ ID NO: 207 - protein, 334 aa, Klebsiella oxytoca
MKLSSLKEEKLISFINCFVETPFLNEIEKDFDRLRYNRFLGGEPQCMLLTGDTGTGKTFLLHHYMSKYPAQNGSGYLRKPLLVSRIPSKPSLESTMVELLKDLGQWGSNYRRNRSSAENLTESLIKCMMRCETELILIDEFQELIENKTRERRNQIANRLKYISETARIPIVLVGMPWAAKISEEPQWSSRLLIRKTIPYFKLTDGLSIFVRVIKGFAARMPFRKPPEIEGKHTILGLYSASQGRMRTLKFLLNEAVKQALSEDSETLTHEHIGKAFHIFYPEHENPFYIPLENIKIYEVREYSGYEIDGAGKEDRLIPQQLTDRIPINQLLRK

SEQ ID NO: 208 - protein, 349 aa, synthetic construct
MKLSSLKEEKLISFINCFVETPFLNEIEKDFDRLRYNRFLGGEPQCMLLTGDTGTGKTFLLHHYMSKYPAQNGSGYLRKPLLVSRIPSKPSLESTMVELLKDLGQWGSNYRRNRSSAENLTESLIKCMMRCETELILIDEFQELIENKTRERRNQIANRLKYISETARIPIVLVGMPWAAKISEEPQWSSRLLIRKTIPYFKLTDGLSIFVRVIKGFAARMPFRKPPEIEGKHTILGLYSASQGRMRTLKFLLNEAVKQALSEDSETLTHEHIGKAFHIFYPEHENPFYIPLENIKIYEVREYSGSGPAAKKKKLDGSGGYEIDGAGKEDRLIPQQLTDRIPINQLLRK

SEQ ID NO: 209 - protein, 407 aa, Klebsiella oxytoca
MHFLIRPEPVCDESLESYLLRLSQDNGFEHYRILSGSLKERLLQSDYEAAGAFPLELAKVNIFHASYSSYLRIRALCLIADLTGQPHTNLLKVTLMHSTVTFGRGHKAVSRDNTHIPLCFIRTNSIPCCPECLAEHGYVRQLWHYKPYTACHRHRRKLLTRCPACHESLNYLYSELLTHCSCGYDLRQAFTPPTSSDDLQLSSMVSDDKCEALSPASASQDKSLRYGALLWFIMRYGESSNNEEGMLSAMHYFRAWPDNFTAELLDMMAAATIKQTKSFNHMSLTDVFGKTLSDCLYLPARDTHRNFILHAFLDYLTNLVMENPRSNIANPGDLLLSIRDAACLLSTSNAQVYRLLDDGFLKVAIRPRAGMKVKISTPVLHLRQVIEFRLTHIPGPHDKGHTYLSAR

SEQ ID NO: 210 - protein, 450 aa, synthetic construct
MPKKKRKVGSGEQKLISEEDLEQKLISEEDLEQKLISEEDLGSGHFLIRPEPVCDESLESYLLRLSQDNGFEHYRILSGSLKERLLQSDYEAAGAFPLELAKVNIFHASYSSYLRIRALCLIADLTGQPHTNLLKVTLMHSTVTFGRGHKAVSRDNTHIPLCFIRTNSIPCCPECLAEHGYVRQLWHYKPYTACHRHRRKLLTRCPACHESLNYLYSELLTHCSCGYDLRQAFTPPTSSDDLQLSSMVSDDKCEALSPASASQDKSLRYGALLWFIMRYGESSNNEEGMLSAMHYFRAWPDNFTAELLDMMAAATIKQTKSFNHMSLTDVFGKTLSDCLYLPARDTHRNFILHAFLDYLTNLVMENPRSNIANPGDLLLSIRDAACLLSTSNAQVYRLLDDGFLKVAIRPRAGMKVKISTPVLHLRQVIEFRLTHIPGPHDKGHTYLSAR

SEQ ID NO: 211 - protein, 209 aa, Pseudoalteromonas arctica
MYIRNLRKPSPNKNVFKFASTKVGNVIMCESTLEFNACFHNEYNDLIESYGSQPEGFKYEFMGKSLPYTPDTVVVYKDKCVKYHEYKYETETAEPLFRERFSAKRAACLKMGVQLILVTENQITKGLALNNFKLLHRYSGVYGIKNIQSEMLNFINKSGAINLVDVKSQFNLSIGEARSFLYALLHKGLLKADLEDDDLSNNPTLWVTP

SEQ ID NO: 212 - protein, 624 aa, Pseudoalteromonas arctica
MGYTMTDFFDEFNESLAPLKPQTPTRYLKLDDANLIKRDLDTFSNTLKNEALQRYKLIISIDKKLSAGWTQRNLDPILDEIFKEDEQARPNWRTVARWRKKYIESNGDLASLVVKNHKMGNRNKRIEGDESFFDKALERFLDAKRPTIATAYQYYKDLIVIENESIVEGKIPIISYTAFNKRIKAIPPYAVAVARHGKFKADQWFAYCAAHVPPTRILERVEIDHTPLDLILLDDELLIPIGRPYLTLLIDVFSGCVLGFHLSYKSPSYVSAAKAIAHAIKPKSLDALNIQLQNDWPCFGKFENLVVDNGAEFWSKNLEHACQSAGINIQYNPVRKPWLKPFIERFFGVMNQYFLPEVPGKTFSNILEKEEYKPEKDAIMRFSTFVEEFHRWIVDVYHQDSNSRETRIPIKRWQQGFDVYPPLTMNEEDEARFTMLMRISDSRTLTRNGIKYQELMYDSTALADYRKHYPQTKETLKKLIKVDPDDISKIYVYLEELESYLEVPCTDPTGYTDGLSIYEHKTIKKVNRETIRESKNSLGLAKARMAIHERVKQEQEVFIASKTKAKITAVKKQAQIADVSNTGKGTIKVSEESAAPVHKNISNDAFDDWDDDLEAFESEQIDNO

SEQ ID NO: 213 - protein, 864 aa, synthetic construct
MYIRNLRKPSPNKNVFKFASTKVGNVIMCESTLEFNACFHNEYNDLIESYGSQPEGFKYEFMGKSLPYTPDTVVVYKDKCVKYHEYKYETETAEPLFRERFSAKRAACLKMGVQLILVTENQITKGLALNNFKLLHRYSGVYGIKNIQSEMLNFINKSGAINLVDVKSQFNLSIGEARSFLYALLHKGLLKADLEDDDLSNNPTLWVTPGSGPKKKRKVGSGYPYDVPDYAYPYDVPDYAYPYDVPDYAGSGTDFFDEFNESLAPLKPQTPTRYLKLDDANLIKRDLDTFSNTLKNEALQRYKLIISIDKKLSAGWTQRNLDPILDEIFKEDEQARPNWRTVARWRKKYIESNGDLASLVVKNHKMGNRNKRIEGDESFFDKALERFLDAKRPTIATAYQYYKDLIVIENESIVEGKIPIISYTAFNKRIKAIPPYAVAVARHGKFKADQWFAYCAAHVPPTRILERVEIDHTPLDLILLDDELLIPIGRPYLTLLIDVFSGCVLGFHLSYKSPSYVSAAKAIAHAIKPKSLDALNIQLQNDWPCFGKFENLVVDNGAEFWSKNLEHACQSAGINIQYNPVRKPWLKPFIERFFGVMNQYFLPEVPGKTFSNILEKEEYKPEKDAIMRFSTFVEEFHRWIVDVYHQDSNSRETRIPIKRWQQGFDVYPPLTMNEEDEARFTMLMRISDSRTLTRNGIKYQELMYDSTALADYRKHYPQTKETLKKLIKVDPDDISKIYVYLEELESYLEVPCTDPTGYTDGLSIYEHKTIKKVNRETIRESKNSLGLAKARMAIHERVKQEQEVFIASKTKAKITAVKKQAQIADVSNTGKGTIKVSEESAAPVHKNISNDAFDDWDDDLEAFE

SEQ ID NO: 214 - protein, 333 aa, Pseudoalteromonas arctica
MNALTEIQIEQLRNFSDCIVMHPQIKAIFNDFDELRLNRKFQSDQQGMLLIGDTGVGKSHTINHYKKRVLATQNYSRNTMPVLISRISRGKGLDATLIQMLADLELFGSSQMKKRGYKTELTKKLVESLIKAQVELLIINEFQELIEFKSVQERQQIANGLKFISEEAKVPIVLVGMPWAAKIAEEPQWASRLVRKRKLEYFSLKNDSKYFRQYLMGLVKQMPFDEPPKLESKHTTMALFAACRGENRALKHLLMEALKLALSCNEYLENKHFIAVYEKFDFFNDKDSLKLKNPFKQDIKDIIIYEVTKNSSYNPNALDPEDMLTGRKFAIVK

SEQ ID NO: 215 - protein, 348 aa, synthetic construct
MNALTEIQIEQLRNFSDCIVMHPQIKAIFNDFDELRLNRKFQSDQQGMLLIGDTGVGKSHTINHYKKRVLATQNYSRNTMPVLISRISRGKGLDATLIQMLADLELFGSSQMKKRGYKTELTKKLVESLIKAQVELLIINEFQELIEFKSVQERQQIANGLKFISEEAKVPIVLVGMPWAAKIAEEPQWASRLVRKRKLEYFSLKNDSKYFRQYLMGLVKQMPFDEPPKLESKHTTMALFAACRGENRALKHLLMEALKLALSCNEYLENKHFIAVYEKFDFFNDKDSLKLKNPFKQDIKDIIIYEVTKNSGSGPAAKKKKLDGSGSYNPNALDPEDMLTGRKFAIVK

SEQ ID NO: 216 - protein, 409 aa, Pseudoalteromonas arctica
MAFLFSPKARAFSDESLESYLLRVVSENFFDSYEGLSLAIREELHELDFEAHGAFPIDLKRLNVYHAKHNSHFRMRALGLLETLLDLPRYELQKLALLKSDIKFNSSAALYKNGVDIPQKFIRYHTEAAVDSIPVCPQCLAEEAYIKQSWHIKWVDACTKHQCTLAHNCPECCAPINYIENESITHCSCGFELTWASTSPVNALSIEHLNKLLDKSERNDSHSLFNNTTLTERFAALLWYQGRYSQTDNFCLDDAVDYFSMWPAVFYKELDELSKNAEMKLIDLFNKTEFKFIFGDAILACPSTQMQRELHFIYRALLDYLVTLVEGNPKAKKPNTADLLVSVLEAATLLGTSVEQVYRLYQDGILQTAFRHKMNQRINPYKGVFFLRHAIEYKTSFGNDKARMYLSAW

SEQ ID NO: 217 - protein, 452 aa, synthetic construct
MPKKKRKVGSGEQKLISEEDLEQKLISEEDLEQKLISEEDLGSGAFLFSPKARAFSDESLESYLLRVVSENFFDSYEGLSLAIREELHELDFEAHGAFPIDLKRLNVYHAKHNSHFRMRALGLLETLLDLPRYELQKLALLKSDIKFNSSAALYKNGVDIPQKFIRYHTEAAVDSIPVCPQCLAEEAYIKQSWHIKWVDACTKHQCTLAHNCPECCAPINYIENESITHCSCGFELTWASTSPVNALSIEHLNKLLDKSERNDSHSLFNNTTLTERFAALLWYQGRYSQTDNFCLDDAVDYFSMWPAVFYKELDELSKNAEMKLIDLFNKTEFKFIFGDAILACPSTQMQRELHFIYRALLDYLVTLVEGNPKAKKPNTADLLVSVLEAATLLGTSVEQVYRLYQDGILQTAFRHKMNQRINPYKGVFFLRHAIEYKTSFGNDKARMYLSAW

SEQ ID NO: 218 - protein, 209 aa, Pseudoalteromonas translucida
MYRRKLKYSRVKNLHKFASQKNKSTCLVESSLEFDACFHFEFSPPIAAFEAQPLGYEYEFDNRICRYTPDFLLTHTDGTQKFIEVKPQSKIADEDFRARFIEKQAIAKQDGRDLILVTDKQIRVYPTLNNLKLLHRYSGFQSLTELQASVLELVKQYGSIKVGQLIRYLKVTAGELLATVLRLLSLGQLFADLTTNEISIETAIWSNNV

SEQ ID NO: 219 - protein, 607 aa, Pseudoalteromonas translucida
MFNNDLFDDEFNQPLPKAETKLPQNYTKDLQALPEKIKTTTFAKLKYIQWLEANIQGGWTQKNLEPLLKLMPDVEGEKKPSWRTAARWYSAYTNADKNIMALIPSHQKKGNRERDTTTDKFFEKALERYLVKEKPSVASAYKFYKDLVIIENDSVVDSVLKPLTYKAFKNRIDNLPQYEVMIARYGKRLADIAYNKVEGHKRPIRVLEKVEIDHTPLDLILLDDELHIPLGRPTLTMLVDVYSHCIVGYYFSFSEPSYDAVRRAMLNAMKPKSEVAKLYPDTINEWKCAGKIETLVVDNGAEFWSNSLELACEEIGINTQYNPVAKPWLKPFVERMFGTINTELLDPVPGKTFSNILQKHEYNPKKDAIMRFTTFMQLFHKWVVDVYHQDADSRFKYIPSQLWDQGFNTLPPTMLSDADLQQLDVVLSISNHRVLRKGGIRLENLSYDSTELANYRKQFSHKVSQEVLIKLNPDDISYIYVYLDKLEHYIKVPCIDPNGYTQNLSLNQHKINIRIHRDFISGSIDNVGLAKARMFIHNKIQNEFEELKNAPKHSKVKGGKALAKHQNISSDSQKSITHSKPVEAKKVTPKEQPTDSWDDFISDLDGF

SEQ ID NO: 220 - protein, 858 aa, synthetic construct
MYRRKLKYSRVKNLHKFASQKNKSTCLVESSLEFDACFHFEFSPPIAAFEAQPLGYEYEFDNRICRYTPDFLLTHTDGTQKFIEVKPQSKIADEDFRARFIEKQAIAKQDGRDLILVTDKQIRVYPTLNNLKLLHRYSGFQSLTELQASVLELVKQYGSIKVGQLIRYLKVTAGELLATVLRLLSLGQLFADLTTNEISIETAIWSNNVGSGPKKKRKVGSGYPYDVPDYAYPYDVPDYAYPYDVPDYAGSGFNNDLFDDEFNQPLPKAETKLPQNYTKDLQALPEKIKTTTFAKLKYIQWLEANIQGGWTQKNLEPLLKLMPDVEGEKKPSWRTAARWYSAYTNADKNIMALIPSHQKKGNRERDTTTDKFFEKALERYLVKEKPSVASAYKFYKDLVIIENDSVVDSVLKPLTYKAFKNRIDNLPQYEVMIARYGKRLADIAYNKVEGHKRPIRVLEKVEIDHTPLDLILLDDELHIPLGRPTLTMLVDVYSHCIVGYYFSFSEPSYDAVRRAMLNAMKPKSEVAKLYPDTINEWKCAGKIETLVVDNGAEFWSNSLELACEEIGINTQYNPVAKPWLKPFVERMFGTINTELLDPVPGKTFSNILQKHEYNPKKDAIMRFTTFMQLFHKWVVDVYHQDADSRFKYIPSQLWDQGFNTLPPTMLSDADLQQLDVVLSISNHRVLRKGGIRLENLSYDSTELANYRKQFSHKVSQEVLIKLNPDDISYIYVYLDKLEHYIKVPCIDPNGYTQNLSLNQHKINIRIHRDFISGSIDNVGLAKARMFIHNKIQNEFEELKNAPKHSKVKGGKALAKHQNISSDSQKSITHSKPVEAKKVTPKEQPTDSWDDFISDLDGF

SEQ ID NO: 221 - protein, 333 aa, Pseudoalteromonas translucida
MLTDKQKEKLNEFRDVFIEYPIITTIFNDFDRLRLGKGLTGEKPCMLLNGDTGTGKTALIKQYKERHLPQFINGVMNHPVLVSRIPSNPTLESTLAELLKDLGQVGSTERKLRINGTRLTTSLIKCLKTCGTELIIIDEFQELIEHNQGKKRREIANRLKYINDEAGVSIVLVGMPWAEKIADEPQWSSRLLIRRQLPYFKLSENPKHFVQLIIGLANRMPFAEKPNLSEQATVFTLFSLSKGCFRTLKYFLDDAVLYALMDNAKTLTTKHLVKAFEVLFPDVPNLFTLPVAEITASEVERYSLYKPESSQDEDPFIATKFTDRMPISQLLRK

SEQ ID NO: 222 - protein, 348 aa, synthetic construct
MLTDKQKEKLNEFRDVFIEYPIITTIFNDFDRLRLGKGLTGEKPCMLLNGDTGTGKTALIKQYKERHLPQFINGVMNHPVLVSRIPSNPTLESTLAELLKDLGQVGSTERKLRINGTRLTTSLIKCLKTCGTELIIIDEFQELIEHNQGKKRREIANRLKYINDEAGVSIVLVGMPWAEKIADEPQWSSRLLIRRQLPYFKLSENPKHFVQLIIGLANRMPFAEKPNLSEQATVFTLFSLSKGCFRTLKYFLDDAVLYALMDNAKTLTTKHLVKAFEVLFPDVPNLFTLPVAEITASEVERYSGSGPAAKKKKLDGSGLYKPESSQDEDPFIATKFTDRMPISQLLRK

SEQ ID NO: 223 - protein, 391 aa, Pseudoalteromonas translucida
MHFLVQTKSYPDEALESYLLRLARDNSYNGYSELADILWQWLAEQDNELEGALPLALSKVDVYHARQASSFRIRALKLVAQLADVNAGDILALAWRRSNFKFGNLAAVSRNELAIPLELLRTDNIPVCIKCLSESSHIPFYWHLKPYKACHKHKSQLITRCKECYDLIDYRASEAFLECVCGCKITNSEQLNDADFKIAIALASSNSQKIVGLISWFAKVKQLDVSDADFNCAFVDYFNTWPESLTTELDLLTNNARLKQLNPFNKTKFSSVYGDLIRDGQIAATSNRKNKVIDEIISYFVELVDSNPKAKHPNIGDLLLCTFDAAVLLNTTTEQVYRLHQEAFLNCAYSQKKHEQLRADSHVFYLRQVIELQQAFAAEKPLTKKQFIAPW

SEQ ID NO: 224 - protein, 434 aa, synthetic construct
MPKKKRKVGSGEQKLISEEDLEQKLISEEDLEQKLISEEDLGSGHFLVQTKSYPDEALESYLLRLARDNSYNGYSELADILWQWLAEQDNELEGALPLALSKVDVYHARQASSFRIRALKLVAQLADVNAGDILALAWRRSNFKFGNLAAVSRNELAIPLELLRTDNIPVCIKCLSESSHIPFYWHLKPYKACHKHKSQLITRCKECYDLIDYRASEAFLECVCGCKITNSEQLNDADFKIAIALASSNSQKIVGLISWFAKVKQLDVSDADFNCAFVDYFNTWPESLTTELDLLTNNARLKQLNPFNKTKFSSVYGDLIRDGQIAATSNRKNKVIDEIISYFVELVDSNPKAKHPNIGDLLLCTFDAAVLLNTTTEQVYRLHQEAFLNCAYSQKKHEQLRADSHVFYLRQVIELQQAFAAEKPLTKKQFIAPW

SEQ ID NO: 225 - protein, 209 aa, Shewanella piezotolerans
MYIRNLRKPSPNKNVFKFASAKVSETIMCESTLEFDACFHHEYNETIETFGSQPKGFYYRFEGKRLPYTPDAILHYIDGTTKFHEYKPYSKTFDPIFRAKFVAKKEAAQALGTELILVTDKQIRVNPILNNLKLLHRYSGIYGVTDIQRELLQLVRKSDNIQLADVASEYNLPIAETRSFLYSLINKGLIKADLNQDDLSCNPSVWCHA

SEQ ID NO: 226 - protein, 614 aa, Shewanella piezotolerans
MDFADEFTESTSAKKPETPAQYVKLDDAELLKRDLDTFPDFLKEKALDKYKLISFIEQENSGGWTQKKLDPILDRLFEGNTEKRPNWRTVVRWRKSYIDSNGDLASLVVKRHKMGNRKKRVEGDEVFFERALSRFLDAKRPKVTTAYQYYKDVITIENETIVDGKIPIISYTAFNQRIKSLPPYPIAVARHGKFKADQWFAYCSSHIPPTRILERVEIDHTPLDLILLDDELLIPLGRPYLTLIVDVFSNCVLGFHLSYKAPSYVSAAKAIVHAIKPKTLSNIGIELQNDWPCYGKFETLVVDNGAEFWSKSLDHACKEAGINIQYNPVRKPWLKPFVERFFGMINQYFLTEIPGKTFSNILEKEDYKPEKDAIMRFSVFVEEFHRWIVDIYHQDSDSRDTRIPIKQWQHGFDIYPPLQMEVEDEKRFNVLMGIADERTLTRNGFKFEELMYDSTALADYRKHYPQTKDTIKKLIKIDPDDLSSIHVYLEELEGYLKVPCTDTTGYTQGLSLHEHKVTKKINREIIRESKDNLGLAKARMAIHARVQQEQELFNESKTKTKLSGVKKKAQLADISSTGKSTIVLPESEPQKSINCNQVEAEMEDDDWDMDLEGY

SEQ ID NO: 227 - protein, 865 aa, synthetic construct
MYIRNLRKPSPNKNVFKFASAKVSETIMCESTLEFDACFHHEYNETIETFGSQPKGFYYRFEGKRLPYTPDAILHYIDGTTKFHEYKPYSKTFDPIFRAKFVAKKEAAQALGTELILVTDKQIRVNPILNNLKLLHRYSGIYGVTDIQRELLQLVRKSDNIQLADVASEYNLPIAETRSFLYSLINKGLIKADLNQDDLSCNPSVWCHAGSGPKKKRKVGSGYPYDVPDYAYPYDVPDYAYPYDVPDYAGSGDFADEFTESTSAKKPETPAQYVKLDDAELLKRDLDTFPDFLKEKALDKYKLISFIEQENSGGWTQKKLDPILDRLFEGNTEKRPNWRTVVRWRKSYIDSNGDLASLVVKRHKMGNRKKRVEGDEVFFERALSRFLDAKRPKVTTAYQYYKDVITIENETIVDGKIPIISYTAFNQRIKSLPPYPIAVARHGKFKADQWFAYCSSHIPPTRILERVEIDHTPLDLILLDDELLIPLGRPYLTLIVDVFSNCVLGFHLSYKAPSYVSAAKAIVHAIKPKTLSNIGIELQNDWPCYGKFETLVVDNGAEFWSKSLDHACKEAGINIQYNPVRKPWLKPFVERFFGMINQYFLTEIPGKTFSNILEKEDYKPEKDAIMRFSVFVEEFHRWIVDIYHQDSDSRDTRIPIKQWQHGFDIYPPLQMEVEDEKRFNVLMGIADERTLTRNGFKFEELMYDSTALADYRKHYPQTKDTIKKLIKIDPDDLSSIHVYLEELEGYLKVPCTDTTGYTQGLSLHEHKVTKKINREIIRESKDNLGLAKARMAIHARVQQEQELFNESKTKTKLSGVKKKAQLADISSTGKSTIVLPESEPQKSINCNQVEAEMEDDDWDMDLEGY

SEQ ID NO: 228 - protein, 330 aa, Shewanella piezotolerans
MTKLTLQQDTALKEFGLCFIELPIVSETFQDFDDLRFNRDYQSDPQCMMLTGETGSGKTRLIQEYRRRVNANSGFRHSDVPVLITNISSNKGLENTLVQILSDLDTFGCHQKKRGMKTDLTKKVVRNLIAANVELLIINEFHDLIKFKNYQEIQIITSALKFISEAANIPIVLVGMPWMKDIINDSEWGSRLRRRKHLEYFSYIRKEDREHFRLLLVGFSKRMSFDTRPVLHSKELTRALFAVCRGEFRQLMVFLYEACKMALQNNDHTLNEKTLAETFDKLGCEHLSSNPFTIKFKEIPIPVLSIPSRYNPNALEEKDEIIDRVFEYIY

SEQ ID NO: 229 - protein, 345 aa, synthetic construct
MTKLTLQQDTALKEFGLCFIELPIVSETFQDFDDLRFNRDYQSDPQCMMLTGETGSGKTRLIQEYRRRVNANSGFRHSDVPVLITNISSNKGLENTLVQILSDLDTFGCHQKKRGMKTDLTKKVVRNLIAANVELLIINEFHDLIKFKNYQEIQIITSALKFISEAANIPIVLVGMPWMKDIINDSEWGSRLRRRKHLEYFSYIRKEDREHFRLLLVGFSKRMSFDTRPVLHSKELTRALFAVCRGEFRQLMVFLYEACKMALQNNDHTLNEKTLAETFDKLGCEHLSSNPFTIKFKEIPIPVLSIPSGSGPAAKKKKLDGSGRYNPNALEEKDEIIDRVFEYIY

SEQ ID NO: 230 - protein, 411 aa, Shewanella piezotolerans
MAFLFSPKSLAFSGESLESYLLRVVAENFFDSYQQLSLAIREELHELDFEAHGAFPIELKRLNVYHAKHNSHFRMRALSLLESLLDLPPHELQKLALLRSNRRFVGGMSAVHRNGIDIPLSFIRCADKDGIESVPICPQCLKEGPYIRQAWHIKPIEVCAKHGCELINHCPDCQQPINYIENESITHCACGFDFTTASSVKADSQAVLLSRSLFDGDALSNNPLLFMGTSVTHRFAALIWYQKCHARNTECMAHRAVGYFEDWPTSFYRELDAVTTGAEARLIDLFNRTSFRSIYGELILDSQCLLPEDKDPHFIYLALMEYISKLVESHPKSKKPNVADMLVTVAEIAVLLSTTHEQVYRLYQDGVLTAGMRSKIRTRISPHIGVFYLRQVIEYKTSFGNDKQGMYLSAW

SEQ ID NO: 231 - protein, 454 aa, synthetic construct
MPKKKRKVGSGEQKLISEEDLEQKLISEEDLEQKLISEEDLGSGAFLFSPKSLAFSGESLESYLLRVVAENFFDSYQQLSLAIREELHELDFEAHGAFPIELKRLNVYHAKHNSHFRMRALSLLESLLDLPPHELQKLALLRSNRRFVGGMSAVHRNGIDIPLSFIRCADKDGIESVPICPQCLKEGPYIRQAWHIKPIEVCAKHGCELINHCPDCQQPINYIENESITHCACGFDFTTASSVKADSQAVLLSRSLFDGDALSNNPLLFMGTSVTHRFAALIWYQKCHARNTECMAHRAVGYFEDWPTSFYRELDAVTTGAEARLIDLFNRTSFRSIYGELILDSQCLLPEDKDPHFIYLALMEYISKLVESHPKSKKPNVADMLVTVAEIAVLLSTTHEQVYRLYQDGVLTAGMRSKIRTRISPHIGVFYLRQVIEYKTSFGNDKQGMYLSAW

SEQ ID NO: 232 - protein, 209 aa, Vibrio azureus
MYIRNLRKPSPNKNIFKFASAKNQGSIMCEGSLERDCCYHFEYDPNVVSFESQPRGFFYDFDGKQLPYTPDFFVVYDDGCHSFMEIKPYSKTLSKEFKLKFQSRKRAAELLGFNLILVTDRQIRAGYFLKNSQMVHRYSGCIADDSLIDIVFAELLLSEVVKISVLARRISGFTLGEVFASVLRLIAVGRAKIDLDLELLNENSTVSVY

SEQ ID NO: 233 - protein, 635 aa, Vibrio azureus
MSRRIKDEFDPAYSEAIEQEFLSHPETIRTQLQYNSLAKTQTYERDLASFPPEQKEKALERYKLLCLIENELRGGWTPRNLDPLIDKYSSNVSIPKPSYKTLIRWQKNFTKSDGNLISLVDKNYLKGNRVARKTGDEAFYERALERFLDSVRPSISAAYQFYCDEITIANEQVISGQVPIVSYQTFKKRIKKEQPYNIVLARHGKYYADKLYHYYQSVKMPTRILERVEIDHTPLDLILLHDDLLIPLGRAYLTLLVDVFSGCIIGFHLGFNAPSYVSVSKAIIHSIKNKDYISNLPIKFENEWLCNGKIENLVVDNGPEFWSKSLDDACTECGINITFNRVKKPWLKPFIERKFGEIIQGIVGWVPGKTFSNVLEKEDYKPDKDAVMRFSVFVEELHRWIVDVHNAKADSRHTRIPNLSWKNSFECLPTKQLSADQEKSFSITMGLLHIGTLTSKGIKYKHLEYDSVALEQYRKQYPQTKESKKKKIKIDPDDLSTIFVFLEELSIYIEVPSKNADGYTDKLSLCVHQRLVKIHREYIKGEINALSLAKARIALHERIQSEQANLKAMSLPERKRKAKGTKKAAKLTGLNSDSSSRTSVNDISMVNEQESSLTKVEPIDDFRSKWNQRRKERSS

SEQ ID NO: 234 - protein, 886 aa, synthetic construct
MYIRNLRKPSPNKNIFKFASAKNQGSIMCEGSLERDCCYHFEYDPNVVSFESQPRGFFYDFDGKQLPYTPDFFVVYDDGCHSFMEIKPYSKTLSKEFKLKFQSRKRAAELLGFNLILVTDRQIRAGYFLKNSQMVHRYSGCIADDSLIDIVFAELLLSEVVKISVLARRISGFTLGEVFASVLRLIAVGRAKIDLDLELLNENSTVSVYGSGPKKKRKVGSGYPYDVPDYAYPYDVPDYAYPYDVPDYAGSGSRRIKDEFDPAYSEAIEQEFLSHPETIRTQLQYNSLAKTQTYERDLASFPPEQKEKALERYKLLCLIENELRGGWTPRNLDPLIDKYSSNVSIPKPSYKTLIRWQKNFTKSDGNLISLVDKNYLKGNRVARKTGDEAFYERALERFLDSVRPSISAAYQFYCDEITIANEQVISGQVPIVSYQTFKKRIKKEQPYNIVLARHGKYYADKLYHYYQSVKMPTRILERVEIDHTPLDLILLHDDLLIPLGRAYLTLLVDVFSGCIIGFHLGFNAPSYVSVSKAIIHSIKNKDYISNLPIKFENEWLCNGKIENLVVDNGPEFWSKSLDDACTECGINITFNRVKKPWLKPFIERKFGEIIQGIVGWVPGKTFSNVLEKEDYKPDKDAVMRFSVFVEELHRWIVDVHNAKADSRHTRIPNLSWKNSFECLPTKQLSADQEKSFSITMGLLHIGTLTSKGIKYKHLEYDSVALEQYRKQYPQTKESKKKKIKIDPDDLSTIFVFLEELSIYIEVPSKNADGYTDKLSLCVHQRLVKIHREYIKGEINALSLAKARIALHERIQSEQANLKAMSLPERKRKAKGTKKAAKLTGLNSDSSSRTSVNDISMVNEQESSLTKVEPIDDFRSKWNQRRKERSS

SEQ ID NO: 235 - protein, 327 aa, Vibrio azureus
MPNSALNYPIDLILSDYHDSFTIYPEVEKVFAGLDWLVRRRNFGSFVPSMLLTGGTGSGKSASIKHYIDNNLSDSEVLLTRVRPTLHETLLWMAKNLNAYRNSRAKPSDIGLMDRVIGCLKKANLKLLIIEECQELFECTSHKERQDIRDRLKMISDDCKLPIVFVGIPSAKLILEDSQWQRRIMVKRELPYVKITDDSSIDRYLDLLEAMQASVPIPFEVDLTDVDSAVRLLAASRGILSNMKELIASAIESSLHLGRQTIRLDDFRLGYEAIYGVDEANPFSINADELVIKQIESYEEYVVDAANGELKFVQQIFNELTIEQLLG

SEQ ID NO: 236 - protein, 342 aa, synthetic construct
MPNSALNYPIDLILSDYHDSFTIYPEVEKVFAGLDWLVRRRNFGSFVPSMLLTGGTGSGKSASIKHYIDNNLSDSEVLLTRVRPTLHETLLWMAKNLNAYRNSRAKPSDIGLMDRVIGCLKKANLKLLIIEECQELFECTSHKERQDIRDRLKMISDDCKLPIVFVGIPSAKLILEDSQWQRRIMVKRELPYVKITDDSSIDRYLDLLEAMQASVPIPFEVDLTDVDSAVRLLAASRGILSNMKELIASAIESSLHLGRQTIRLDDFRLGYEAIYGVDEANPFSINADELVIKQIESYEGSGPAAKKKKLDGSGEYVVDAANGELKFVQQIFNELTIEQLLG

SEQ ID NO: 237 - protein, 363 aa, Vibrio azureus
MDQHEAIAGAFPLELNRVNIYHAQTTSQMRVRVLIHLENQFKLNNFGVLRLALSHSKAQFSPQYKAVHRFGVDYPYAFLRKRFTPICPLCIDEAPYIRQQWQFISDQVCQYHGCKLIHRCPECKSRLEYQSAESINQCECGYELRNSPIEDAPEAELLVAQWLSGNNSKPLWLLKAEMTISERYGFLLWYVNRYGEFDELSFESFIEYCSDWPTVLWQELDGLKEKAEVVRVKNWKKMFFNEAFGSLLKDCRQLPSRQLNHNIVLKQVLAYFTRLIATVPSSAKGNIGDLLLSPLEASTLLSCTTDEVYRLYEFGEIKAAIRPRIHTKIANHESAFTLRSVIETKLTRMSSESDGLNVYLPEW

SEQ ID NO: 238 - protein, 406 aa, synthetic construct
MPKKKRKVGSGEQKLISEEDLEQKLISEEDLEQKLISEEDLGSGDQHEAIAGAFPLELNRVNIYHAQTTSQMRVRVLIHLENQFKLNNFGVLRLALSHSKAQFSPQYKAVHRFGVDYPYAFLRKRFTPICPLCIDEAPYIRQQWQFISDQVCQYHGCKLIHRCPECKSRLEYQSAESINQCECGYELRNSPIEDAPEAELLVAQWLSGNNSKPLWLLKAEMTISERYGFLLWYVNRYGEFDELSFESFIEYCSDWPTVLWQELDGLKEKAEVVRVKNWKKMFFNEAFGSLLKDCRQLPSRQLNHNIVLKQVLAYFTRLIATVPSSAKGNIGDLLLSPLEASTLLSCTTDEVYRLYEFGEIKAAIRPRIHTKIANHESAFTLRSVIETKLTRMSSESDGLNVYLPEW

SEQ ID NO: 239 - protein, 207 aa, Vibrio fluvialis
MYVRNLRKPSANKNVYKFVSLKNGCTIMCESSLEYDCCYYLEYSDDVVRYQSQPKGYRFPYRGKQHPYTPDFLVHKKDGTSYLLEVKPLSKTFSSEFQDMFHQKQIMASELGVPLLLVTDRQIRNDVHLNNLKLVHRYSGFIENSSHLESVWSAVSQSSSICIKALPEILNLTIGEVFASVLRLIGLGKAKTKLDVLLDENSLISVA

SEQ ID NO: 240 - protein, 635 aa, Vibrio fluvialis
MSFGPFEDEFGSITNDVQQQYDASPEAKLSRLKYSPLESSKVIERDLSSFPEEQKLKALERYKLISLIAKEINGGWTPKNLIPLIDKHIEKLSIPKPSDRTVKRWYKAFCESDGDIKSLVDSHHLKGNRQPRIEDDEPLFIEAVERFLDAVRPSYSKAYQVYCDRIEIENSSIVSGEIAKVSYEAFKKRIKKLPPYTIALKRHGKYYADKLFNYYEAVKMPTRILERVEIDHTPLDLILLDDELLVPLGRAYLTLLVDVFSGCIIGFHLGFKAPSYTAVSKAIIHSVKSKEYVNELPIGLSNQWICHGKIENLVVDNGAEFWSKSLDQACIEAGINIIYNKVRKPWLKPFVERKFGELIQGIVGWIPGRTFSNVLEKEDYDPQKDAVMRFSVFVEELHRWIIDVHNASADSRHTRIPNYHWKKSEEVMPPPALTERDEIQFRVIMGVVHKGALTSKGIKFKHLMYDNVALEHYRKQYPQSKDSRIKTIKIDPDDLSRIFVFLEEREGYIEVPCKCDPLGYTKKLSLCEHLRTVKVHRDFIKGQVDSLSLAKARQALHERIKQEHENLRQMSLPQRAKKAKNGKKMAELAGVSSDSPKSITTDYPIEDIIQPHESTPVDDLQSLWNKRRALRKSSK

SEQ ID NO: 241 - protein, 884 aa, synthetic construct
MYVRNLRKPSANKNVYKFVSLKNGCTIMCESSLEYDCCYYLEYSDDVVRYQSQPKGYRFPYRGKQHPYTPDFLVHKKDGTSYLLEVKPLSKTFSSEFQDMFHQKQIMASELGVPLLLVTDRQIRNDVHLNNLKLVHRYSGFIENSSHLESVWSAVSQSSSICIKALPEILNLTIGEVFASVLRLIGLGKAKTKLDVLLDENSLISVAGSGPKKKRKVGSGYPYDVPDYAYPYDVPDYAYPYDVPDYAGSGSFGPFEDEFGSITNDVQQQYDASPEAKLSRLKYSPLESSKVIERDLSSFPEEQKLKALERYKLISLIAKEINGGWTPKNLIPLIDKHIEKLSIPKPSDRTVKRWYKAFCESDGDIKSLVDSHHLKGNRQPRIEDDEPLFIEAVERFLDAVRPSYSKAYQVYCDRIEIENSSIVSGEIAKVSYEAFKKRIKKLPPYTIALKRHGKYYADKLFNYYEAVKMPTRILERVEIDHTPLDLILLDDELLVPLGRAYLTLLVDVFSGCIIGFHLGFKAPSYTAVSKAIIHSVKSKEYVNELPIGLSNQWICHGKIENLVVDNGAEFWSKSLDQACIEAGINIIYNKVRKPWLKPFVERKFGELIQGIVGWIPGRTFSNVLEKEDYDPQKDAVMRFSVFVEELHRWIIDVHNASADSRHTRIPNYHWKKSEEVMPPPALTERDEIQFRVIMGVVHKGALTSKGIKFKHLMYDNVALEHYRKQYPQSKDSRIKTIKIDPDDLSRIFVFLEEREGYIEVPCKCDPLGYTKKLSLCEHLRTVKVHRDFIKGQVDSLSLAKARQALHERIKQEHENLRQMSLPQRAKKAKNGKKMAELAGVSSDSPKSITTDYPIEDIIQPHESTPVDDLQSLWNKRRALRKSSK

SEQ ID NO: 242 - protein, 328 aa, Vibrio fluvialis
MTLLQPTNNDVDTLLADFHQSFVVYPDVEKVFEGLDWIVRRSQFGKFAPSMLITGGTGAGKTSVVETYLNNHFSASEVLVTRVRPSFVETLVWAIEKLNVPYNSRSKRSEIGLQDYFISSVKKSKLKLLVIEEAQELFECASPKERQKIRDRLKMISDECRLPIVFIGIPTAKLILEDSQWDRRIMVKRDLPYIRITNEESLDIYIALLEGLEKTLSISVVPELSDMDMAMRLLAASKGMIGLIKELVGYALELALLEGKRQITQNEFIQAFKSIFGPDISNPFEIELDKLLISQIIEYEGYILDSDSGDIKFTHQIFEDIPLTELLR

SEQ ID NO: 243 - protein, 343 aa, synthetic construct
MTLLQPTNNDVDTLLADFHQSFVVYPDVEKVFEGLDWIVRRSQFGKFAPSMLITGGTGAGKTSVVETYLNNHFSASEVLVTRVRPSFVETLVWAIEKLNVPYNSRSKRSEIGLQDYFISSVKKSKLKLLVIEEAQELFECASPKERQKIRDRLKMISDECRLPIVFIGIPTAKLILEDSQWDRRIMVKRDLPYIRITNEESLDIYIALLEGLEKTLSISVVPELSDMDMAMRLLAASKGMIGLIKELVGYALELALLEGKRQITQNEFIQAFKSIFGPDISNPFEIELDKLLISQIIEYEGSGPAAKKKKLDGSGGYILDSDSGDIKFTHQIFEDIPLTELLR

SEQ ID NO: 244 - protein, 403 aa, Vibrio fluvialis
MDTEIEVYPDESLESFLLRLSKYQGYERFSHFAEDIWQSTIQQHQAISGAFPFELSRINIYKAQTTSQMRVRVLIDLERRLKLSDFGILRLALAHSNANFSSDYKAVHRYGVDYPQAFLRKRFIPVCPKCLDEAPYIRQLWHFVPYQACHKHHGQLVQRCPECGKLFDYQSSELIEHCECGLSLTNIEPEQESDSTFIVARWLAGEKYIEPGLMSQQLTLSSRYGFLLWYINRYSELDEISFDNFVECCKTWPQKLDADLDSIVLKADIVRTRTWSKTYFGEVFGPLLKECRNLPSRELSKNPVLQSIVQYFSRLVANYPRDRTANIGDVLVSPLEASTLVSCSTDEIYRLYQFGELKAQLTPKLHTKIENHHSVFTLRSIIELKFSRMCSETDGLNHYLPEW

SEQ ID NO: 245 - protein, 446 aa, synthetic construct
MPKKKRKVGSGEQKLISEEDLEQKLISEEDLEQKLISEEDLGSGDTEIEVYPDESLESFLLRLSKYQGYERFSHFAEDIWQSTIQQHQAISGAFPFELSRINIYKAQTTSQMRVRVLIDLERRLKLSDFGILRLALAHSNANFSSDYKAVHRYGVDYPQAFLRKRFIPVCPKCLDEAPYIRQLWHFVPYQACHKHHGQLVQRCPECGKLFDYQSSELIEHCECGLSLTNIEPEQESDSTFIVARWLAGEKYIEPGLMSQQLTLSSRYGFLLWYINRYSELDEISFDNFVECCKTWPQKLDADLDSIVLKADIVRTRTWSKTYFGEVFGPLLKECRNLPSRELSKNPVLQSIVQYFSRLVANYPRDRTANIGDVLVSPLEASTLVSCSTDEIYRLYQFGELKAQLTPKLHTKIENHHSVFTLRSIIELKFSRMCSETDGLNHYLPEW

SEQ ID NO: 246 - protein, 208 aa, Vibrio natriegens
MYIRNLRKPSPNKNIFKFSSLKNRDAVMCEGSLEKDCCYHFEYDPDVVRYESQPEGFYYDFNGKKRPYTPDFLVTYHDGTFEYVEVKPYSKTLSKTFKQEFSARKEAANRRGVGLVLVTDKQIRDGYFLKNTELVHRYSGCIAGDELAIKVYSNLVAQNTMKISDLADSIGESFGRVFASVLRLIAVGKAGADLDIAQLSESTTVSVR

SEQ ID NO: 247 - protein, 638 aa, Vibrio natriegens
MVGRFHDEFEPENNEDSDRKHEFLPETQTERLKYSRLQSTQIIERDLSSYPEEQKNKALERYKLLCLVANELSGGWTPKNLTPLIEKHFDKTRLTKKPSYKSLQRWHNSFVDSDGSFTSLVDKNHLKGNRGARVVGDEKYYDEALKMFLDARRQSIRAAHAFYCDRITVANEAIVAGRIPKVSYEAFKDRIRKEEPYSVALARHGKYYADKLFNYYQSVEMPTRILERVEMDHTPLDLILLHDDLMVPLGRAHLTLLVDVFSGCIIGFHLGFKAPSYVSASRAVIHATKSKTYISEMPIVFNNEWLCEGKIENLVVDNGAEFWSKSWEDACLEVGINVVYNKVRKPWLKPFVERKFGEIVQGIVGWVPGKTFSNVLEKEDYRPEKDAVMRFSTFVEEFHRWIVDVHNVNADSRYKRIPNLYWKQSYDVLPPLKLLPDQEQAFSVVMGILHHRKLTDKGIKFMHLEYDCVALSDYRKTYPQTNESSKKKIKVDPDDLSAIYVYLDELQGYVKVPSKDPIGYTVRLSVCEHEKILAAHRTYIKGEMDVLSLAKARLALHDRIESEQADLMQLTHNERKRKAKSTKKIAEISSVNSDTPHSKLSDRTPKPNVSISESESNSDTTPLESFRSKWNERKNRRE

SEQ ID NO: 248 - protein, 888 aa, synthetic construct
MYIRNLRKPSPNKNIFKFSSLKNRDAVMCEGSLEKDCCYHFEYDPDVVRYESQPEGFYYDFNGKKRPYTPDFLVTYHDGTFEYVEVKPYSKTLSKTFKQEFSARKEAANRRGVGLVLVTDKQIRDGYFLKNTELVHRYSGCIAGDELAIKVYSNLVAQNTMKISDLADSIGESFGRVFASVLRLIAVGKAGADLDIAQLSESTTVSVRGSGPKKKRKVGSGYPYDVPDYAYPYDVPDYAYPYDVPDYAGSGVGRFHDEFEPENNEDSDRKHEFLPETQTERLKYSRLQSTQIIERDLSSYPEEQKNKALERYKLLCLVANELSGGWTPKNLTPLIEKHFDKTRLTKKPSYKSLQRWHNSFVDSDGSFTSLVDKNHLKGNRGARVVGDEKYYDEALKMFLDARRQSIRAAHAFYCDRITVANEAIVAGRIPKVSYEAFKDRIRKEEPYSVALARHGKYYADKLFNYYQSVEMPTRILERVEMDHTPLDLILLHDDLMVPLGRAHLTLLVDVFSGCIIGFHLGFKAPSYVSASRAVIHATKSKTYISEMPIVFNNEWLCEGKIENLVVDNGAEFWSKSWEDACLEVGINVVYNKVRKPWLKPFVERKFGEIVQGIVGWVPGKTFSNVLEKEDYRPEKDAVMRFSTFVEEFHRWIVDVHNVNADSRYKRIPNLYWKQSYDVLPPLKLLPDQEQAFSVVMGILHHRKLTDKGIKFMHLEYDCVALSDYRKTYPQTNESSKKKIKVDPDDLSAIYVYLDELQGYVKVPSKDPIGYTVRLSVCEHEKILAAHRTYIKGEMDVLSLAKARLALHDRIESEQADLMQLTHNERKRKAKSTKKIAEISSVNSDTPHSKLSDRTPKPNVSISESESNSDTTPLESFRSKWNERKNRRE

SEQ ID NO: 249 - protein, 338 aa, Vibrio natriegens
MERAQKPEGIVVTTARRNLDRDEVLADYHDSFSVYPEVEKVLSGLEWIIKRRKFGTFAPSMLLTAGTGAGKTATINHFIEKNLSRNEVLITRVKPSLLETLLWMAKELGAYRNSRAKPSEIGLTDCVIETSKRVGLKLLVIEECQELFERTSHNQRQDIRDRLKMISDECHLPIVFVGLHSAGLILEDSQWNRRIMVRRTLPYIKITDESAIDNYLDVLQALEKTVPLPFKVPLTDVDFAMRLLSASKGILGEIKELIAAALEVTLEKNKDCIDEEDFAAVYEKINDPNDINPFTVQIDALTIEQIASYENYVTDAETGELRFVKQVFSKLSIQQLVG

SEQ ID NO: 250 - protein, 353 aa, synthetic construct
MERAQKPEGIVVTTARRNLDRDEVLADYHDSFSVYPEVEKVLSGLEWIIKRRKFGTFAPSMLLTAGTGAGKTATINHFIEKNLSRNEVLITRVKPSLLETLLWMAKELGAYRNSRAKPSEIGLTDCVIETSKRVGLKLLVIEECQELFERTSHNQRQDIRDRLKMISDECHLPIVFVGLHSAGLILEDSQWNRRIMVRRTLPYIKITDESAIDNYLDVLQALEKTVPLPFKVPLTDVDFAMRLLSASKGILGEIKELIAAALEVTLEKNKDCIDEEDFAAVYEKINDPNDINPFTVQIDALTIEQIASYEGSGPAAKKKKLDGSGNYVTDAETGELRFVKQVFSKLSIQQLVG

SEQ ID NO: 251 - protein, 403 aa, Vibrio natriegens
VETDIQLYPDESLESFLLRLSQEQSYERFSHFAEDIWQNTLLQHEAISGAFPFELSRINIYKAQTTSQMRVRVLIDLEKQLGLTNFGVLRLALAHSKASFSPEYKAVHRFGVDYPQAFLRKRFAPVCSQCLEESPYIRQLWQFIPYQACHKHHCKLVHQCPECGNRLEYQHSELIEHCDCGFRLASCQAETANHASLTVAQWLAGEEVDKSGIFNQLLTQSSRFGFLLWYVNRYGDVDNISLEDFVRCCETWPQRLNEDLDAIVEKADMLRIQPWHKTYFCEVFSELLKECRHLPSREIGKNPVLQSVVQYFTELVTKYPRTKAANIADMLLSPLEASTLLSCSTDEILRLYQFGQLKAQFTPKLHGKIENHHSVFILRSIIELKLSRMCSETDGLMHYLPEW

SEQ ID NO: 252 - protein, 446 aa, synthetic construct
MPKKKRKVGSGEQKLISEEDLEQKLISEEDLEQKLISEEDLGSGETDIQLYPDESLESFLLRLSQEQSYERFSHFAEDIWQNTLLQHEAISGAFPFELSRINIYKAQTTSQMRVRVLIDLEKQLGLTNFGVLRLALAHSKASFSPEYKAVHRFGVDYPQAFLRKRFAPVCSQCLEESPYIRQLWQFIPYQACHKHHCKLVHQCPECGNRLEYQHSELIEHCDCGFRLASCQAETANHASLTVAQWLAGEEVDKSGIFNQLLTQSSRFGFLLWYVNRYGDVDNISLEDFVRCCETWPQRLNEDLDAIVEKADMLRIQPWHKTYFCEVFSELLKECRHLPSREIGKNPVLQSVVQYFTELVTKYPRTKAANIADMLLSPLEASTLLSCSTDEILRLYQFGQLKAQFTPKLHGKIENHHSVFILRSIIELKLSRMCSETDGLMHYLPEW

SEQ ID NO: 253 - protein, 700 aa, Aeromonas salmonicida
MHIEELLDIEDHGERDRQLRRYLAPYSAEIGVDGAEKMALVVLLNLTLKRDRVESLCDEGLARQLLSDEGHITNCLHTVRWLHTHNLKYPDARVSGERLIINAPPLIPGVISSAGLPMRMGWAHDSSDINLAKLFGTSFRYRDDSTNLALQLVARSKTWEQALIGLGLTQQQLDIWCQLLASNLENNTFPTVVSPFSKQVRFLYQGNYCVVTPVVSHALLAQLQNVVHEKKLQCTYIHHDHPASVGSLVGALGGKVAVLDYPPPVSPDKARSFSQARKHRLANGQSLFDRSVFNDHVFIDALKHVISRPGLTRKQQRQLRLSALRYLRRQLAIWLGPIIEWRDEIVSSGRGEPGNLPSGGLELELITQPKKMLPELMLQVAGRFHLELQNHSAGRRFAFHPALMAPIKSQILWLLRQLADDEEKDEPHPPTSCYYLHLSGLTVYDASALANPYLCGIPSLSALAGFCHDYERRLQSLIGQSVYFRGLAWYLGRYSLVTGKHLPEPSKSADPKSVSAIRRPGLLDGRYCDLGMDLIIEVHIPTGGSLPFTTCLDLLRVALPARFAGGCLHPPSLYEEYNWCTVYQDKSTLFTVLSRLPRYGCWIYPSDADLRSFEELSEALALDRRLRPVATGFVFLEEPVERAGSIEGQHVYAESAIGTALCINPVEMRLAGKKRFFGAGFWQLNDAKGAILMNGSANTG

SEQ ID NO: 254 - protein, 737 aa, synthetic construct
MPKKKRKVGSGDYKDDDDKDYKDDDDKDYKDDDDKGSGHIEELLDIEDHGERDRQLRRYLAPYSAEIGVDGAEKMALVVLLNLTLKRDRVESLCDEGLARQLLSDEGHITNCLHTVRWLHTHNLKYPDARVSGERLIINAPPLIPGVISSAGLPMRMGWAHDSSDINLAKLFGTSFRYRDDSTNLALQLVARSKTWEQALIGLGLTQQQLDIWCQLLASNLENNTFPTVVSPFSKQVRFLYQGNYCVVTPVVSHALLAQLQNVVHEKKLQCTYIHHDHPASVGSLVGALGGKVAVLDYPPPVSPDKARSFSQARKHRLANGQSLFDRSVFNDHVFIDALKHVISRPGLTRKQQRQLRLSALRYLRRQLAIWLGPIIEWRDEIVSSGRGEPGNLPSGGLELELITQPKKMLPELMLQVAGRFHLELQNHSAGRRFAFHPALMAPIKSQILWLLRQLADDEEKDEPHPPTSCYYLHLSGLTVYDASALANPYLCGIPSLSALAGFCHDYERRLQSLIGQSVYFRGLAWYLGRYSLVTGKHLPEPSKSADPKSVSAIRRPGLLDGRYCDLGMDLIIEVHIPTGGSLPFTTCLDLLRVALPARFAGGCLHPPSLYEEYNWCTVYQDKSTLFTVLSRLPRYGCWIYPSDADLRSFEELSEALALDRRLRPVATGFVFLEEPVERAGSIEGQHVYAESAIGTALCINPVEMRLAGKKRFFGAGFWQLNDAKGAILMNGSANTG

SEQ ID NO: 255 - protein, 347 aa, Aeromonas salmonicida
MELCTHLSYSRSLSPGKAVFFYKTAESDFVPLRIEVAKISGQKCGYTEGFDANLKPKNIERYELAYSNPQTIEACYVPPNVDELYCRFSLRVEANSMRPYVCSNPDVLRVMIGLAQAYQRLGGYNELARRYSANVLRGIWLWRNQYTQGTKIEIKTSLGSTYHIPDARRLSWSGDWPELEQKQLEQLTSEMAKALSQPDIFWFADVTASLKTGFCQEIFPSQKFTERPDDHSVASRQLATVECSDGQLAACINPQKIGAALQKIDDWWANDADLPLRVHEYGANHEALTALRHPATGQDFYHLLTKAEQFVTVLESSEGGGVELPGEVHYLMAVLVKGGLFQKGKGR

SEQ ID NO: 256 - protein, 357 aa, synthetic construct
MPKKKRKVGSGELCTHLSYSRSLSPGKAVFFYKTAESDFVPLRIEVAKISGQKCGYTEGFDANLKPKNIERYELAYSNPQTIEACYVPPNVDELYCRFSLRVEANSMRPYVCSNPDVLRVMIGLAQAYQRLGGYNELARRYSANVLRGIWLWRNQYTQGTKIEIKTSLGSTYHIPDARRLSWSGDWPELEQKQLEQLTSEMAKALSQPDIFWFADVTASLKTGFCQEIFPSQKFTERPDDHSVASRQLATVECSDGQLAACINPQKIGAALQKIDDWWANDADLPLRVHEYGANHEALTALRHPATGQDFYHLLTKAEQFVTVLESSEGGGVELPGEVHYLMAVLVKGGLFQKGKGR

SEQ ID NO: 257 - protein, 206 aa, Aeromonas salmonicida
MTENRYFFAIRYLSDDVDCGLLAGRCISILHGFRQAHPGIQIGVAFPEWSDRDLGRSIAFVSTNKSLLERFRERSYFQVMQADNFFALSLVLEVPDTCQNVRFIRNQNLAKLFVGERRRRLARAKRRAKARGEAFQPHMPDETKVVGVFHSVFMQSASSGQSYILHIQKHRYERSEDSGYSSYGLASNDLYTGYVPDLGAIFSTLF

SEQ ID NO: 258 - protein, 216 aa, synthetic construct
MPKKKRKVGSGTENRYFFAIRYLSDDVDCGLLAGRCISILHGFRQAHPGIQIGVAFPEWSDRDLGRSIAFVSTNKSLLERFRERSYFQVMQADNFFALSLVLEVPDTCQNVRFIRNQNLAKLFVGERRRRLARAKRRAKARGEAFQPHMPDETKVVGVFHSVFMQSASSGQSYILHIQKHRYERSEDSGYSSYGLASNDLYTGYVPDLGAIFSTLF

SEQ ID NO: 259 - protein, 640 aa, Vibrio cholerae
MQTLKELIASNPDDLTTELKRAFRPLTPHIAIDGNELDALTILVNLTDKTDDQKDLLDRAKCKQKLRDEKWWASCINCVNYRQSHNPKFPDIRSEGVIRTQALGELPSFLLSSSKIPPYHWSYSHDSKYVNKSAFLTNEFCWDGEISCLGELLKDADHPLWNTLKKLGCSQKTCKAMAKQLADITLTTINVTLAPNYLTQISLPDSDTSYISLSPVASLSMQSHFHQRLQDENRHSAITRFSRTTNMGVTAMTCGGAFRMLKSGAKFSSPPHHRLNSKRSWLTSEHVQSLKQYQRLNKSLIPENSRIALRRKYKIELQNMVRSWFAMQDHTLDSNILIQHLNHDLSYLGATKRFAYDPAMTKLFTELLKRELSNSINNGEQHTNGSFLVLPNIRVCGATALSSPVTVGIPSLTAFFGFVHAFERNINRTTSSFRVESFAICVHQLHVEKRGLTAEFVEKGDGTISAPATRDDWQCDVVFSLILNTNFAQHIDQDTLVTSLPKRLARGSAKIAIDDFKHINSFSTLETAIESLPIEAGRWLSLYAQSNNNLSDLLAAMTEDHQLMASCVGYHLLEEPKDKPNSLRGYKHAIAECIIGLINSITFSSETDPNTIFWSLKNYQNYLVVQPRSINDETTDKSSL

SEQ ID NO: 260 - protein, 677 aa, synthetic construct
MPKKKRKVGSGDYKDDDDKDYKDDDDKDYKDDDDKGSGQTLKELIASNPDDLTTELKRAFRPLTPHIAIDGNELDALTILVNLTDKTDDQKDLLDRAKCKQKLRDEKWWASCINCVNYRQSHNPKFPDIRSEGVIRTQALGELPSFLLSSSKIPPYHWSYSHDSKYVNKSAFLTNEFCWDGEISCLGELLKDADHPLWNTLKKLGCSQKTCKAMAKQLADITLTTINVTLAPNYLTQISLPDSDTSYISLSPVASLSMQSHFHQRLQDENRHSAITRFSRTTNMGVTAMTCGGAFRMLKSGAKFSSPPHHRLNSKRSWLTSEHVQSLKQYQRLNKSLIPENSRIALRRKYKIELQNMVRSWFAMQDHTLDSNILIQHLNHDLSYLGATKRFAYDPAMTKLFTELLKRELSNSINNGEQHTNGSFLVLPNIRVCGATALSSPVTVGIPSLTAFFGFVHAFERNINRTTSSFRVESFAICVHQLHVEKRGLTAEFVEKGDGTISAPATRDDWQCDVVFSLILNTNFAQHIDQDTLVTSLPKRLARGSAKIAIDDFKHINSFSTLETAIESLPIEAGRWLSLYAQSNNNLSDLLAAMTEDHQLMASCVGYHLLEEPKDKPNSLRGYKHAIAECIIGLINSITFSSETDPNTIFWSLKNYQNYLVVQPRSINDETTDKSSL

SEQ ID NO: 261 - protein, 352 aa, Vibrio cholerae
MKLPTNLAYERSIDPSDVCFFVVWPDDRKTPLTYNSRTLLGQMEAASLAYDVSGQPIKSATAEALAQGNPHQVDFCHVPYGASHIECSFSVSFSSELRQPYKCNSSKVKQTLVQLVELYETKIGWTELATRYLMNICNGKWLWKNTRKAYCWNIVLTPWPWNGEKVGFEDIRTNYTSRQDFKNNKNWSAIVEMIKTAFSSTDGLAIFEVRATLHLPTNAMVRPSQVFTEKESGSKSKSKTQNSRVFQSTTIDGERSPILGAFKTGAAIATIDDWYPEATEPLRVGRFGVHREDVTCYRHPSTGKDFFSILQQAEHYIEVLSANKTPAQETINDMHFLMANLIKGGMFQHKGD

SEQ ID NO: 262 - protein, 362 aa, synthetic construct
MPKKKRKVGSGKLPTNLAYERSIDPSDVCFFVVWPDDRKTPLTYNSRTLLGQMEAASLAYDVSGQPIKSATAEALAQGNPHQVDFCHVPYGASHIECSFSVSFSSELRQPYKCNSSKVKQTLVQLVELYETKIGWTELATRYLMNICNGKWLWKNTRKAYCWNIVLTPWPWNGEKVGFEDIRTNYTSRQDFKNNKNWSAIVEMIKTAFSSTDGLAIFEVRATLHLPTNAMVRPSQVFTEKESGSKSKSKTQNSRVFQSTTIDGERSPILGAFKTGAAIATIDDWYPEATEPLRVGRFGVHREDVTCYRHPSTGKDFFSILQQAEHYIEVLSANKTPAQETINDMHFLMANLIKGGMFQHKGD

SEQ ID NO: 263 - protein, 199 aa, Vibrio cholerae
MKWYYKTITFLPELCNNESLAAKCLRVLHGFNYQYETRNIGVSFPLWCDATVGKKISFVSKNKIELDLLLKQHYFVQMEQLQYFHISNTVLVPEDCTYVSFRRCQSIDKLTAAGLARKIRRLEKRALSRGEQFDPSSFAQKEHTAIAHYHSLGESSKQTNRNFRLNIRMLSEQPREGNSIFSSYGLSNSENSFQPVPLI

SEQ ID NO: 264 - protein, 209 aa, synthetic construct
MPKKKRKVGSGKWYYKTITFLPELCNNESLAAKCLRVLHGFNYQYETRNIGVSFPLWCDATVGKKISFVSKNKIELDLLLKQHYFVQMEQLQYFHISNTVLVPEDCTYVSFRRCQSIDKLTAAGLARKIRRLEKRALSRGEQFDPSSFAQKEHTAIAHYHSLGESSKQTNRNFRLNIRMLSEQPREGNSIFSSYGLSNSENSFQPVPLI

SEQ ID NO: 265 - protein, 695 aa, Vibrio cholerae
MTKLSDLLAIEDEAIKQTALKKMFMPYTEDVCVDGYEQETLTILLNLSSSHQADRCSDWLDVARAQRYLKDRENLDASLAEIQWFHTHNLKFPDCRVKDQRIIARPLSTAEEFISSAVLDQRLGWAHNSAVYRHTLWLLNPFKWQSQPVCILLLIQQKNPVWLDLLTEFGLDVKSLARLQRAIEEQLPENSFPDSVSTYSKQLRFPWGDDYVSITPVVSHALQCELEIRARSPENKFSFVSSSLPNSASIGNLCGSLGGYMRVLNYPLGVKQAKGGTLTENRQKSGHYFDDYQVTNAKICQVLNRLIGSEPSKTQRQRERARKVRSKILRKQIALWMLPLIELRDIAESEPNQQQLEHDDTLAQAFLSLPEWELGSLAGEFNRRLHLAFQNNIYSAKFAYHPKLMQVAKAQVTWVLEQLSKPINNQDTVTGEQYIYLSSMRVQDAVAMSNPCLCGVPSLTAIWGFMHDYQRQFNQLVNNDSPVEFSSFAFYVRNENIQSTAKLTEPNSIAKARTVSNAKRPTIRSKRLADLEIDLVIRVHSESRISDFRSALKTALPVAFAGGALYQPQLSTQIEWLRTFTGRSELFHVLKGLPAYGRWLYPSEKQPTNFDELERLLTQDDDNLLVSLGYHLLEHPTKRDNAITGCHAYAENAIGLAKRINPIEVRFSGRDHFLNHAFWSIECSSETILIKNYRD

SEQ ID NO: 266 - protein, 732 aa, synthetic construct
MPKKKRKVGSGDYKDDDDKDYKDDDDKDYKDDDDKGSGTKLSDLLAIEDEAIKQTALKKMFMPYTEDVCVDGYEQETLTILLNLSSSHQADRCSDWLDVARAQRYLKDRENLDASLAEIQWFHTHNLKFPDCRVKDQRIIARPLSTAEEFISSAVLDQRLGWAHNSAVYRHTLWLLNPFKWQSQPVCILLLIQQKNPVWLDLLTEFGLDVKSLARLQRAIEEQLPENSFPDSVSTYSKQLRFPWGDDYVSITPVVSHALQCELEIRARSPENKFSFVSSSLPNSASIGNLCGSLGGYMRVLNYPLGVKQAKGGTLTENRQKSGHYFDDYQVTNAKICQVLNRLIGSEPSKTQRQRERARKVRSKILRKQIALWMLPLIELRDIAESEPNQQQLEHDDTLAQAFLSLPEWELGSLAGEFNRRLHLAFQNNIYSAKFAYHPKLMQVAKAQVTWVLEQLSKPINNQDTVTGEQYIYLSSMRVQDAVAMSNPCLCGVPSLTAIWGFMHDYQRQFNQLVNNDSPVEFSSFAFYVRNENIQSTAKLTEPNSIAKARTVSNAKRPTIRSKRLADLEIDLVIRVHSESRISDFRSALKTALPVAFAGGALYQPQLSTQIEWLRTFTGRSELFHVLKGLPAYGRWLYPSEKQPTNFDELERLLTQDDDNLLVSLGYHLLEHPTKRDNAITGCHAYAENAIGLAKRINPIEVRFSGRDHFLNHAFWSIECSSETILIKNYRD

SEQ ID NO: 267 - protein, 343 aa, Vibrio cholerae
MELCTQLNYVRSLSAGKAYFYYLSESGEMCPLDVDRTRLRAPKGSYSEAYKGNKFVDKNVAPQDLAYSNPQFIEECYVKPGVDEIYCAFSLRIRANSLTPDMCSDDEVRSKLSMLAKIYKDLNGYKELAHRYAKNILLGTWLWRNRECRNITIEVTTSELDTFVVEHAQKLSWYGHWDGDSTECLERLTAYLERALSDPTEYFYMDVKAKMRVGWGDEVYPSQEFLDSREDGIPTKQLATVELLSGKETVAFHGQKVGAALQSIDDWWNENADKPLRVNEYGADREYVIARRHVTHGNDFYQLVRNTENWIETMTASRTIPNDVHFIMSVLIKGGLFNCAKAN

SEQ ID NO: 268 - protein, 353 aa, synthetic construct
MPKKKRKVGSGELCTQLNYVRSLSAGKAYFYYLSESGEMCPLDVDRTRLRAPKGSYSEAYKGNKFVDKNVAPQDLAYSNPQFIEECYVKPGVDEIYCAFSLRIRANSLTPDMCSDDEVRSKLSMLAKIYKDLNGYKELAHRYAKNILLGTWLWRNRECRNITIEVTTSELDTFVVEHAQKLSWYGHWDGDSTECLERLTAYLERALSDPTEYFYMDVKAKMRVGWGDEVYPSQEFLDSREDGIPTKQLATVELLSGKETVAFHGQKVGAALQSIDDWWNENADKPLRVNEYGADREYVIARRHVTHGNDFYQLVRNTENWIETMTASRTIPNDVHFIMSVLIKGGLFNCAKAN

SEQ ID NO: 269 - protein, 209 aa, Vibrio cholerae
MSQRYYFLIRYTNANADYGLLAGRCISQMHLFMVNHHQAMNRVGVSFPDWNESSVGQTIAFVSEDKEMMIGLSFQPYFSLMVNEGLFEISSVYEVPDTSAEVRFVRNQTIGKNFLGSKKRRIKRSMARAELFGVEQSLPVTNEDRVIDSFHRIPISSGSSRQDFILFIQKELADERAKSGFNSYGFATNQEKRATVPDLRFNLFEEDSF

SEQ ID NO: 270 - protein, 219 aa, synthetic construct
MPKKKRKVGSGSQRYYFLIRYTNANADYGLLAGRCISQMHLFMVNHHQAMNRVGVSFPDWNESSVGQTIAFVSEDKEMMIGLSFQPYFSLMVNEGLFEISSVYEVPDTSAEVRFVRNQTIGKNFLGSKKRRIKRSMARAELFGVEQSLPVTNEDRVIDSFHRIPISSGSSRQDFILFIQKELADERAKSGFNSYGFATNQEKRATVPDLRFNLFEEDSF

SEQ ID NO: 271 - protein, 689 aa, Aliivibrio wodanis
MEFTDILIIQDVKERNRAFKVAFAHYSSAIFIDDHEVEAITCLLNLCTPKTEDYLDKTSASLFLNNHDNIQKCLDELKWFHSHNVKYPDCRVKGQSIISLPIDSVSNTINSNVVPYRLGWSHDSGKVNYTHFLLSCFKWRGVQTTLSQLFITDTLFWLDIIKKIQCNWTKKQTEQFIHSIQKEMPAKTLPEDISPYSKQILFPYKNDYLTLTPVTSNSIQTWLEHQSRKPNDIRWIKRESKHPASVGALSSSIGGYHSLLSSLPSTSQSPHSYHDNMTSKTECREAFCASAITEKSTTDALQRLISSEVRMNVKHRKQIRKSGIHFIRQKIALWLTPLIRWRDHIDNNQIQITNDHPSLVNLFLSSPIANFPDLLTPLHNHLNQTLGNNKYTKRFAYHPDLMPIFKSQISWILNKLTQDENINQQPVLTRTQFIHLKNLRLYNGNALSSPYVCGLPSLTGFWGFMHDFERRLKTKIEENIHFEAFSLFVHQYELQSSPPLCEASDVYKKRELSPAKRLLTQPSYSCDMRFDLIIKVHTEVNLSDISQRMQSAMPARCVGGTLHQPSLHESLEWLRTYTSSEHLFEELACLPNSGRWIYPPSETFNTPDEFLSILGNSTHLAICNGYSFLEDPTYRENVSLNQHVFCEPLIGLAEQVIPIDMRLNRQKHYFSNAFWSINSDFNSILISKA

SEQ ID NO: 272 - protein, 726 aa, synthetic construct
MPKKKRKVGSGDYKDDDDKDYKDDDDKDYKDDDDKGSGEFTDILIIQDVKERNRAFKVAFAHYSSAIFIDDHEVEAITCLLNLCTPKTEDYLDKTSASLFLNNHDNIQKCLDELKWFHSHNVKYPDCRVKGQSIISLPIDSVSNTINSNVVPYRLGWSHDSGKVNYTHFLLSCFKWRGVQTTLSQLFITDTLFWLDIIKKIQCNWTKKQTEQFIHSIQKEMPAKTLPEDISPYSKQILFPYKNDYLTLTPVTSNSIQTWLEHQSRKPNDIRWIKRESKHPASVGALSSSIGGYHSLLSSLPSTSQSPHSYHDNMTSKTECREAFCASAITEKSTTDALQRLISSEVRMNVKHRKQIRKSGIHFIRQKIALWLTPLIRWRDHIDNNQIQITNDHPSLVNLFLSSPIANFPDLLTPLHNHLNQTLGNNKYTKRFAYHPDLMPIFKSQISWILNKLTQDENINQQPVLTRTQFIHLKNLRLYNGNALSSPYVCGLPSLTGFWGFMHDFERRLKTKIEENIHFEAFSLFVHQYELQSSPPLCEASDVYKKRELSPAKRLLTQPSYSCDMRFDLIIKVHTEVNLSDISQRMQSAMPARCVGGTLHQPSLHESLEWLRTYTSSEHLFEELACLPNSGRWIYPPSETFNTPDEFLSILGNSTHLAICNGYSFLEDPTYRENVSLNQHVFCEPLIGLAEQVIPIDMRLNRQKHYFSNAFWSINSDFNSILISKA

SEQ ID NO: 273 - protein, 344 aa, Aliivibrio wodanis
MKLCNNLNYTRSLSPGKAVFYYESKDGQMNPIKCEQTHLRAPKAGFSEAFNSDYSTKNTAPQDLSFSNPQFIEECYVPVGIDEIKIRFSLRIEANSLQPDKCSDIQIREILQAFATKYKENGGYQELGERYAKNLLSGTWLWRNEHNLGTSISIKTTSNQEFNINNAFKLSRKTSAKDKKTISKLGSEIASALSDPDHYYFADITATINVAFCQEIYPSQEFLDTKEKGKPSKVYAKTSLQTGEKTIAFHAQKIGAAIQLIDDWWADDADIPLRVNEFGADHHNVIARRHPSHRNDFYTLIQNADNYCAQLNENSDITDDMHYVMAVLVKGGLFQKSASSKKGK

SEQ ID NO: 274 - protein, 354 aa, synthetic construct
MPKKKRKVGSGKLCNNLNYTRSLSPGKAVFYYESKDGQMNPIKCEQTHLRAPKAGFSEAFNSDYSTKNTAPQDLSFSNPQFIEECYVPVGIDEIKIRFSLRIEANSLQPDKCSDIQIREILQAFATKYKENGGYQELGERYAKNLLSGTWLWRNEHNLGTSISIKTTSNQEFNINNAFKLSRKTSAKDKKTISKLGSEIASALSDPDHYYFADITATINVAFCQEIYPSQEFLDTKEKGKPSKVYAKTSLQTGEKTIAFHAQKIGAAIQLIDDWWADDADIPLRVNEFGADHHNVIARRHPSHRNDFYTLIQNADNYCAQLNENSDITDDMHYVMAVLVKGGLFQKSASSKKGK

SEQ ID NO: 275 - protein, 213 aa, Aliivibrio wodanis
MLTHYFSITYVPDDCDNELLAGRCIAEFHKFISSLRLIENNSFAIGFPNWSEQSIGNEFAIFSDNSELLSAIKYQPYFNLMKSEELFSITDIKPVPNNLPQIRFIRNQSIGKIFIGSKKRRIQRSITRNNKEHTPISNEDREFDTFHKVSCSSKSKQQQFILHIQKDITPRTTDSNDSYNSYGLATNSKHLGTVPDLSKIPFYCEDKLSNKDQ

SEQ ID NO: 276 - protein, 223 aa, synthetic construct
MPKKKRKVGSGLTHYFSITYVPDDCDNELLAGRCIAEFHKFISSLRLIENNSFAIGFPNWSEQSIGNEFAIFSDNSELLSAIKYQPYFNLMKSEELFSITDIKPVPNNLPQIRFIRNQSIGKIFIGSKKRRIQRSITRNNKEHTPISNEDREFDTFHKVSCSSKSKQQQFILHIQKDITPRTTDSNDSYNSYGLATNSKHLGTVPDLSKIPFYCEDKLSNKDQ

SEQ ID NO: 277 - protein, 694 aa, Parashewanella spongiae
MLTINELLEIADIEERNKAIRSRLRPFHEPLNVDGSEKEILIVLLNLGYSSKEQVDLLEQKSAQQFLKGEELFGKTISEAEWIHTHNLKYPDIRVSKQTIRATLPEDVEGVCSKDILESIELGWSHNATFVGKVTPLITEFKWQGKVTCLINLLLSESAFWVNLLITLGVSKRWVNRTKIQLADITANSFPEEVDRYSPQLRFYNQRGYVSVTPVTNHKLLSEIQKRCFNKEFRCRKVKHPRATCAGHLITSLGGYVSVLAYYPDRGFNRNINQYIDDKTDSNFFNSKYLNNHNFLEALGELVFSPKRETLKLTRIARVAAIKSIRQTLYWWLAKATDYKKHANISSDVSSNAKLFKRYLNQGESKNELASELSNLIHEQLAQANQTKQFAYHSKLISPIKRQLQFLLKNRANSETEQQEQRVFYLHLKRLRVEDLETLSCPYLWGMPSIIAFAGFAHKFELNLKKLGFHNIRVMGVACFVHLYQVTAKTSLPAYSHLKKEKQSDQLRPTRPALVSAPKSQMLFDLVLRLWNGGNEYNLESLPNPVQIREALPTRYAGGTIFPTIRKLEERFTTSHNLTELFNSLSFMPAKGCWLYPSQFKVHSLDELHKALDTDLNLRPVAIGYQYLEEPKYRDGGISELHCYAENLLGLTRCTNSVDVRVGGAQRFLREAFWAQKTTDSEVLMVKSRFEFKL

SEQ ID NO: 278 - protein, 731 aa, synthetic construct
MPKKKRKVGSGDYKDDDDKDYKDDDDKDYKDDDDKGSGLTINELLEIADIEERNKAIRSRLRPFHEPLNVDGSEKEILIVLLNLGYSSKEQVDLLEQKSAQQFLKGEELFGKTISEAEWIHTHNLKYPDIRVSKQTIRATLPEDVEGVCSKDILESIELGWSHNATFVGKVTPLITEFKWQGKVTCLINLLLSESAFWVNLLITLGVSKRWVNRTKIQLADITANSFPEEVDRYSPQLRFYNQRGYVSVTPVTNHKLLSEIQKRCFNKEFRCRKVKHPRATCAGHLITSLGGYVSVLAYYPDRGFNRNINQYIDDKTDSNFFNSKYLNNHNFLEALGELVFSPKRETLKLTRIARVAAIKSIRQTLYWWLAKATDYKKHANISSDVSSNAKLFKRYLNQGESKNELASELSNLIHEQLAQANQTKQFAYHSKLISPIKRQLQFLLKNRANSETEQQEQRVFYLHLKRLRVEDLETLSCPYLWGMPSIIAFAGFAHKFELNLKKLGFHNIRVMGVACFVHLYQVTAKTSLPAYSHLKKEKQSDQLRPTRPALVSAPKSQMLFDLVLRLWNGGNEYNLESLPNPVQIREALPTRYAGGTIFPTIRKLEERFTTSHNLTELFNSLSFMPAKGCWLYPSQFKVHSLDELHKALDTDLNLRPVAIGYQYLEEPKYRDGGISELHCYAENLLGLTRCTNSVDVRVGGAQRFLREAFWAQKTTDSEVLMVKSRFEFKL

SEQ ID NO: 279 - protein, 344 aa, Parashewanella spongiae
MKIPTHLSYMRSLSPSPALFFYKTDESDFNPIEVFSEGINGRMSGSAVAYNKDGKLKNVTANDLGHANLHASEYCYVPPKIKEFYCKFSLTIAPNSLSPYICNDQDLVMYLEKLAQCYAEKGGYQELATRYAKNILNGLWLWRNKKSPKVDISVYDFLSEQEVANTAGVQSLSWDGNWGKYHDELQKLSKIIAQALHNNEACELEVVATIRNRFMQEIYPSQLLPEENKVHKQLATTRVEDGSETTCLGRFKVGAAIQIIDDWHGGDKPLRVSSYGSVPERLVALRTPSNKKDVYSLLPKIIDYINFLESNNLGENETSNEINYLMAMLVKGDVLGMGSEKKSK

SEQ ID NO: 280 - protein, 354 aa, synthetic construct
MPKKKRKVGSGKIPTHLSYMRSLSPSPALFFYKTDESDFNPIEVFSEGINGRMSGSAVAYNKDGKLKNVTANDLGHANLHASEYCYVPPKIKEFYCKFSLTIAPNSLSPYICNDQDLVMYLEKLAQCYAEKGGYQELATRYAKNILNGLWLWRNKKSPKVDISVYDFLSEQEVANTAGVQSLSWDGNWGKYHDELQKLSKIIAQALHNNEACELEVVATIRNRFMQEIYPSQLLPEENKVHKQLATTRVEDGSETTCLGRFKVGAAIQIIDDWHGGDKPLRVSSYGSVPERLVALRTPSNKKDVYSLLPKIIDYINFLESNNLGENETSNEINYLMAMLVKGDVLGMGSEKKSK

SEQ ID NO: 281 - protein, 199 aa, Parashewanella spongiae
MRSYFYITYLPENVNNELLAARCVNVLHGFVAKEDVVDIGISFPAWSEHTVGNQLAFVSTSKSKLTRILHHNYFSMMKEDGLFYISNIEPVPTGLKEIQFLRNNTIAKTTLGEKRRRNKRAFERAEARGDEYAPVQNNQAQFIHNYHILNCTSGSKNMSFPLYIQKREDTSHQNCDFNHYGLASNKLYSGTVPEFNFDQ

SEQ ID NO: 282 - protein, 209 aa, synthetic construct
MPKKKRKVGSGRSYFYITYLPENVNNELLAARCVNVLHGFVAKEDVVDIGISFPAWSEHTVGNQLAFVSTSKSKLTRILHHNYFSMMKEDGLFYISNIEPVPTGLKEIQFLRNNTIAKTTLGEKRRRNKRAFERAEARGDEYAPVQNNQAQFIHNYHILNCTSGSKNMSFPLYIQKREDTSHQNCDFNHYGLASNKLYSGTVPEFNFDQ

SEQ ID NO: 283 - protein, 683 aa, Pseudoalteromonas sp.
MNLQDAFAIESLKEKTTALRKLFTPYMSHVAVDGFEEQALTVLINLVYKRSEIDDLTSTRTAKSVLRDEVLLSKCINEVKWFHTHNLKYPDIRVSHQRLISKVVSEDIAGICSRSLPLSFGWSHNSAEINHAKLFLTSFTWQGEVTCLANLLINEEPVWINLIRTYGFTKKAVLGIAGKIKQLLPVAELPLEVSSFSPQLQMPFQQSYLAVTPVVSHAMLAKIQQLTTDRKLNFGLVEHSRPANVGDLASSVGGNIRVLRYFPKTYSKAVNCSEVFNNDSEKAFKIRALLNSQFQQALLVLVGIKQFNTLRQKRLARVAAIRQVRVSLQLWLDNILEAKNNAQGQAYPEWAKHYLDQSITNCISQFSNVLNESLGNLSKLKRFAYHPNLMGVFKTQLNYVFTHCIPDEETLNDEQIVYVHCQDMRVFDAEAMANPYIQGMPSLTALNGLAHNFERKLKNFIDPSIKCIGSAINIESYQLHTGKPLPEPSKLKQVAGRSHVIRSGIIDKPKCDITLDLVFRLFVPNIKLLDKLNSQLVKPALPSMFAGGTMHPPSLYQNIDWCHLHTKPSELFKNIKAKSLNGSWLYPSKKVVKSFEQLIDALNGNFNLRPAAIGFAALEEPIKRDVALHEYHCYAEPVIGLLECVSNTSVKYAGAKQFFHDAFWVMDVQKESMLMKKSKFEYE

SEQ ID NO: 284 - protein, 720 aa, synthetic construct
MPKKKRKVGSGDYKDDDDKDYKDDDDKDYKDDDDKGSGNLQDAFAIESLKEKTTALRKLFTPYMSHVAVDGFEEQALTVLINLVYKRSEIDDLTSTRTAKSVLRDEVLLSKCINEVKWFHTHNLKYPDIRVSHQRLISKVVSEDIAGICSRSLPLSFGWSHNSAEINHAKLFLTSFTWQGEVTCLANLLINEEPVWINLIRTYGFTKKAVLGIAGKIKQLLPVAELPLEVSSFSPQLQMPFQQSYLAVTPVVSHAMLAKIQQLTTDRKLNFGLVEHSRPANVGDLASSVGGNIRVLRYFPKTYSKAVNCSEVFNNDSEKAFKIRALLNSQFQQALLVLVGIKQFNTLRQKRLARVAAIRQVRVSLQLWLDNILEAKNNAQGQAYPEWAKHYLDQSITNCISQFSNVLNESLGNLSKLKRFAYHPNLMGVFKTQLNYVFTHCIPDEETLNDEQIVYVHCQDMRVFDAEAMANPYIQGMPSLTALNGLAHNFERKLKNFIDPSIKCIGSAINIESYQLHTGKPLPEPSKLKQVAGRSHVIRSGIIDKPKCDITLDLVFRLFVPNIKLLDKLNSQLVKPALPSMFAGGTMHPPSLYQNIDWCHLHTKPSELFKNIKAKSLNGSWLYPSKKVVKSFEQLIDALNGNFNLRPAAIGFAALEEPIKRDVALHEYHCYAEPVIGLLECVSNTSVKYAGAKQFFHDAFWVMDVQKESMLMKKSKFEYE

SEQ ID NO: 285 - protein, 342 aa, Pseudoalteromonas sp.
MQLPRHLSYTRSLSPSKAVFFYKTSESDFEPLQIEQNKLVGQKSGFGDAYQKQNVAKNLAPQDLAFGNPQTIDVCYVPPAVNELFCRFSLRVEANSNEPHVCDDPKVIYWLKRFFETYKKHNGLNEVATRYAKNILMGNWLWRNRQSPNVDIEILTEHAAPIIVEGAQKLKWQGNWQNNQTALITLSEAIQEGLSNPQNYCYLDITAKIKNAFSQEVHPSQKFVDNVEQGMSSKQLAYTQVGDKKAASLNSQKVGAAIQTIDDWYEGGYKPLRTHEYGADKQILVAHRTPKSHSDFYSLLPRIALHIKHMEKHGLEQSEESNAVHFIAAVLIKGGLFQRSKA

SEQ ID NO: 286 - protein, 352 aa, synthetic construct
MPKKKRKVGSGQLPRHLSYTRSLSPSKAVFFYKTSESDFEPLQIEQNKLVGQKSGFGDAYQKQNVAKNLAPQDLAFGNPQTIDVCYVPPAVNELFCRFSLRVEANSNEPHVCDDPKVIYWLKRFFETYKKHNGLNEVATRYAKNILMGNWLWRNRQSPNVDIEILTEHAAPIIVEGAQKLKWQGNWQNNQTALITLSEAIQEGLSNPQNYCYLDITAKIKNAFSQEVHPSQKFVDNVEQGMSSKQLAYTQVGDKKAASLNSQKVGAAIQTIDDWYEGGYKPLRTHEYGADKQILVAHRTPKSHSDFYSLLPRIALHIKHMEKHGLEQSEESNAVHFIAAVLIKGGLFQRSKA

SEQ ID NO: 287 - protein, 199 aa, Pseudoalteromonas sp.
MKRYYFTITYLPKNCDVSLLAGRCIGILHGFMSSREISNIGVCFPKWNEQEIGNELAFVSTDKKQLTNLSQQSYFEMMAQDKLFGLSKILEVPTNQNEVMFIRNQSVAKAFVGEKQRRLKRAKKRAEARGEVYNPEYQFEAKDIGHFHSIPVSSKANGQSYVLHIQKIENTNATENQFNNYGFATNQTFQGTVPSLNTQ

SEQ ID NO: 288 - protein, 209 aa, synthetic construct
MPKKKRKVGSGKRYYFTITYLPKNCDVSLLAGRCIGILHGFMSSREISNIGVCFPKWNEQEIGNELAFVSTDKKQLTNLSQQSYFEMMAQDKLFGLSKILEVPTNQNEVMFIRNQSVAKAFVGEKQRRLKRAKKRAEARGEVYNPEYQFEAKDIGHFHSIPVSSKANGQSYVLHIQKIENTNATENQFNNYGFATNQTFQGTVPSLNTQ

SEQ ID NO: 289 - protein, 696 aa, Vibrio diazotrophicus
MTTLQDLIDIEDSKLRFIEIKKAFMPYTRPVEVDGSEKQALIVLLNLSLSKPEVKDWLDFPRALDYFADSDNLSAAEQEIQWFHTHNLKFPDCRVSEQRIIATPLYTETPTLTSQSLNRAYGWAHNSAVYKHTIWLLNEFRWRGRVENLLNLICGGDDFWLELLADMGLKPKAQIQLKDLIEHQLPLTHFPDEVNRYSKQLRFPWRGDYLSVTPVVSHAIQQQLSVLSRQGECSLRFKTMTYPNSASIGNLCGSLGGYINVLNYPIDVIANRHQTLGASRSRTKRYFDDFQLTSKSTCSVLAHLTGFEQPQMRKAQKHVRQYQLKIIRKQIALWLLPLIELRDNSVTDPIGFYDEPDDELAKRFLTINELDFIELTTSLNQRLNIALQNNRFASRFAYHPKLMRVLKTELIWVLTQLSQPEPEPPTVSDSKVQYLYLSSMRVFDAAAMSCPYLSGAPSLTAVWGFVHRYQRELQDLLSDGEGQFEFKDFAFFIRDESVQTSAKLTEPSVIAKARSISQVKRTTIIREDCSDLIFDIVIAIESDQRISDYQSQFKAALPTNFAGGALFQPEINSGINWLRTFVSKSELFQAVKGLPGYGTWLSPDSFQPQNLAELQECLTIDSSLIPVSNGFHFLGSPQERKGALTKLHCYAENNIALAKRTNPIEVRFAGSDHFFEQVFWSLEVTEQTILIKNKRI

SEQ ID NO: 290 - protein, 733 aa, synthetic construct
MPKKKRKVGSGDYKDDDDKDYKDDDDKDYKDDDDKGSGTTLQDLIDIEDSKLRFIEIKKAFMPYTRPVEVDGSEKQALIVLLNLSLSKPEVKDWLDFPRALDYFADSDNLSAAEQEIQWFHTHNLKFPDCRVSEQRIIATPLYTETPTLTSQSLNRAYGWAHNSAVYKHTIWLLNEFRWRGRVENLLNLICGGDDFWLELLADMGLKPKAQIQLKDLIEHQLPLTHFPDEVNRYSKQLRFPWRGDYLSVTPVVSHAIQQQLSVLSRQGECSLRFKTMTYPNSASIGNLCGSLGGYINVLNYPIDVIANRHQTLGASRSRTKRYFDDFQLTSKSTCSVLAHLTGFEQPQMRKAQKHVRQYQLKIIRKQIALWLLPLIELRDNSVTDPIGFYDEPDDELAKRFLTINELDFIELTTSLNQRLNIALQNNRFASRFAYHPKLMRVLKTELIWVLTQLSQPEPEPPTVSDSKVQYLYLSSMRVFDAAAMSCPYLSGAPSLTAVWGFVHRYQRELQDLLSDGEGQFEFKDFAFFIRDESVQTSAKLTEPSVIAKARSISQVKRTTIIREDCSDLIFDIVIAIESDQRISDYQSQFKAALPTNFAGGALFQPEINSGINWLRTFVSKSELFQAVKGLPGYGTWLSPDSFQPQNLAELQECLTIDSSLIPVSNGFHFLGSPQERKGALTKLHCYAENNIALAKRTNPIEVRFAGSDHFFEQVFWSLEVTEQTILIKNKRI

SEQ ID NO: 291 - protein, 345 aa, Vibrio diazotrophicus
MELCSQLNYVRSLSPGRAYFYYLDEDNKMRPLQIDRTHLRAPKSGYSEAFSGNFKSKNIAPQDLSYSNPQFIEECYVPPGVNDIYCAFSLRVRANSLSPEVCVDNEVRDILCNFAALYKELGGYRELARRYAKNILMGTWVWRNRECRNIRVEVKTEDKEWVITDARFLDWYGSWEKDSQLALDEFTDYLSQALSDRTCYFNMDIKAKLTVGWGDEVYPSQEFLDVKEAGKPSKLLAKVTVNGEESAAFHSQKVGAAIQRIDDWWDENADKPLRVNEYGADKEYAIARRHSSRHRDFYSLIAHTESYVELMLETNLISDDVHFIMAVLTKGGVFSGASKKSKKDE

SEQ ID NO: 292 - protein, 355 aa, synthetic construct
MPKKKRKVGSGELCSQLNYVRSLSPGRAYFYYLDEDNKMRPLQIDRTHLRAPKSGYSEAFSGNFKSKNIAPQDLSYSNPQFIEECYVPPGVNDIYCAFSLRVRANSLSPEVCVDNEVRDILCNFAALYKELGGYRELARRYAKNILMGTWVWRNRECRNIRVEVKTEDKEWVITDARFLDWYGSWEKDSQLALDEFTDYLSQALSDRTCYFNMDIKAKLTVGWGDEVYPSQEFLDVKEAGKPSKLLAKVTVNGEESAAFHSQKVGAAIQRIDDWWDENADKPLRVNEYGADKEYAIARRHSSRHRDFYSLIAHTESYVELMLETNLISDDVHFIMAVLTKGGVFSGASKKSKKDE

SEQ ID NO: 293 - protein, 203 aa, Vibrio diazotrophicus
MESRYYFSIRYIPEHVDNELLAGRCISNMHGFLSHERNTQFKNSVGICFPLWNEQTVGNVITFVSTNESILTGLSYQPYFSTMMNENLFEISGIRIVPDDAKDVRFVFNKTIQKIFNGSKKRRIKRAMKRAEEFGHTFTPISVEVREFELFHEIPINSKSSGRDFVLHIQRQNPVEAEIGQGFNGYGFASNQLWRRTVPLILF

SEQ ID NO: 294 - protein, 213 aa, synthetic construct
MPKKKRKVGSGESRYYFSIRYIPEHVDNELLAGRCISNMHGFLSHERNTQFKNSVGICFPLWNEQTVGNVITFVSTNESILTGLSYQPYFSTMMNENLFEISGIRIVPDDAKDVRFVFNKTIQKIFNGSKKRRIKRAMKRAEEFGHTFTPISVEVREFELFHEIPINSKSSGRDFVLHIQRQNPVEAEIGQGFNGYGFASNQLWRRTVPLILF

SEQ ID NO: 295 - protein, 699 aa, Shewanella sp.
MVDKLKFHELLDIDDISERNIALRRAFTGYTVPMDVTGNEASALTILLNLTYPRKRVDDLLDKRLAKQTLNTDAHLDASIDEVQWLHTHNLKYPDIRVSKQRLITASPLSHSHILSSANCISTLGWSHDSAKVNLAKLFSCHFNWQDRVCCLATLLSDPPKIWKEAFQALGMLVKDFMNLCGRIKASLPSYESPSRVDKYSIQVRLPYRDGYLAITPVVSHALQAEIQQAAMAKQCRYTNFEFTRPAAVSELSASLGGNVKALNYPPRIGNAVHGLSDSWLLKFQAGQTVLNQGALSQPRFKRALEGLLSNGFELALKQRRLHKVASMRQIRATLTEWLSPLLEWRLEVEENKNNVSELACIHGSFEYQFLTAQKENLVGLLNPMFSLLNTILSNSNTLQKYAFHQRLMRPLKCSLKWLLDNLSKESNAIDSDEDNQQRYLYLKGIRVFDAQALSNPYCAGLPSLTAVWGMVHNYQRRLNKRLGTQLRLTSFSWFIRQYSSVAGKKLPEYGMQGQKENQFRRAGIVDNKHCDLVFDLVVHIDGYEEDLDAIDNSTDAIKASFPATFAGGVMHPPEIGSVDEWCELYPSETSLYSKLRRLPASGKWVMPTRYQMDSLDGLLQLLKLNVALCPVMSGYLMLGPPESRKNSLEPLHCYAEPAIGVVECATAIDIRLQGMSNFFRRAFWMLDIKETSMLMKRI

SEQ ID NO: 296 - protein, 736 aa, synthetic construct
MPKKKRKVGSGDYKDDDDKDYKDDDDKDYKDDDDKGSGVDKLKFHELLDIDDISERNIALRRAFTGYTVPMDVTGNEASALTILLNLTYPRKRVDDLLDKRLAKQTLNTDAHLDASIDEVQWLHTHNLKYPDIRVSKQRLITASPLSHSHILSSANCISTLGWSHDSAKVNLAKLFSCHFNWQDRVCCLATLLSDPPKIWKEAFQALGMLVKDFMNLCGRIKASLPSYESPSRVDKYSIQVRLPYRDGYLAITPVVSHALQAEIQQAAMAKQCRYTNFEFTRPAAVSELSASLGGNVKALNYPPRIGNAVHGLSDSWLLKFQAGQTVLNQGALSQPRFKRALEGLLSNGFELALKQRRLHKVASMRQIRATLTEWLSPLLEWRLEVEENKNNVSELACIHGSFEYQFLTAQKENLVGLLNPMFSLLNTILSNSNTLQKYAFHQRLMRPLKCSLKWLLDNLSKESNAIDSDEDNQQRYLYLKGIRVFDAQALSNPYCAGLPSLTAVWGMVHNYQRRLNKRLGTQLRLTSFSWFIRQYSSVAGKKLPEYGMQGQKENQFRRAGIVDNKHCDLVFDLVVHIDGYEEDLDAIDNSTDAIKASFPATFAGGVMHPPEIGSVDEWCELYPSETSLYSKLRRLPASGKWVMPTRYQMDSLDGLLQLLKLNVALCPVMSGYLMLGPPESRKNSLEPLHCYAEPAIGVVECATAIDIRLQGMSNFFRRAFWMLDIKETSMLMKRI

SEQ ID NO: 297 - protein, 365 aa, Shewanella sp.
MELCNVLKYDRSLYPGKAVFFYKTAESDFVPLEAEINRIRGQKAGFTEAFTPQFKSKNLAPQDLAHCNPLILEECYVPPNVEYIYCRFSLRVQANSLKPAGCSEPTVFALLEEFAAIFKACGGYKELATRYCKNVLLGTWLWRNQNTGNSQIDIKTSAGNCYQIANTRQLAWDSRWPADAQQVLEELSDEVHQALTDPTVFWHANITAKIETAFCQEIYPSQSFGEKAAQGEASKQFAKVKCVDGRYAVSFNSVKIGAALQLIDDWWDVDDSKRLRIHEYGADKELGVARRAPESKQSFYSLFINTELYLAELNQQLAEDEYSISPNIYYLFAVLIKGGMFQKKAEAKSKSKAETSTAKITPAKA

SEQ ID NO: 298 - protein, 375 aa, synthetic construct
MPKKKRKVGSGELCNVLKYDRSLYPGKAVFFYKTAESDFVPLEAEINRIRGQKAGFTEAFTPQFKSKNLAPQDLAHCNPLILEECYVPPNVEYIYCRFSLRVQANSLKPAGCSEPTVFALLEEFAAIFKACGGYKELATRYCKNVLLGTWLWRNQNTGNSQIDIKTSAGNCYQIANTRQLAWDSRWPADAQQVLEELSDEVHQALTDPTVFWHANITAKIETAFCQEIYPSQSFGEKAAQGEASKQFAKVKCVDGRYAVSFNSVKIGAALQLIDDWWDVDDSKRLRIHEYGADKELGVARRAPESKQSFYSLFINTELYLAELNQQLAEDEYSISPNIYYLFAVLIKGGMFQKKAEAKSKSKAETSTAKITPAKA

SEQ ID NO: 299 - protein, 199 aa, Shewanella sp.
MHRYYFMVRFLPEQANLALLMGRCISIMHGFICKHDIQGLGVSFPAWSDASIGNMIAFVHTDIAALNELKLQGYFQDMQECGVFKVDNVEAVPDDCVEVRFKRNQGIAKMFVGEARRRLKRLEKRALARGEVFNPNKNDEPRELDCFHCIAIGSTSTEQDFLLHVQKEIVQKYEEPEFNQYGLATNKLLRGTVPEFSEF

SEQ ID NO: 300 - protein, 209 aa, synthetic construct
MPKKKRKVGSGHRYYFMVRFLPEQANLALLMGRCISIMHGFICKHDIQGLGVSFPAWSDASIGNMIAFVHTDIAALNELKLQGYFQDMQECGVFKVDNVEAVPDDCVEVRFKRNQGIAKMFVGEARRRLKRLEKRALARGEVFNPNKNDEPRELDCFHCIAIGSTSTEQDFLLHVQKEIVQKYEEPEFNQYGLATNKLLRGTVPEFSEF

SEQ ID NO: 301 - protein, 695 aa, Pseudoalteromonas sp.
MHLKELLEITDTTERDRSLRRAFSPYTAMIDITGSEAVALIILLNLTYRKNQVDDLLDKKLAKQALKSEDHINKCIKEIAWFHTHNLKYPDIRVSKQNLAVEPPTLHSYVLSSANYPKAYGWSHNSAKVNFAKLFVSYFKWQNQVSWLAQVLATNSDNWKSAFTSLGLSVKAFKSLCVTVKNSLPEEAIPDSVDRYSRQIRMPYHDGYLAVTPVISHVVQSKIQQAAIDKRARFSNVEFTRPAAVSMLAASLGGVINVLNYPPYIRSKYHGLSNSRAFKLNNGQTVFNVEALLKPELIKALEGIIFSNNALALKQRRQQKVKNIKELRNTLLEWFSPVFEWRLDAIENGYDLEQLESASERLEYKILSLPDNELPSLTIPLFRLLNEMLGGVSMTQRYAFHPKLMSPLKAALQWLLVNLTDQKHVLIEEDDEHYRYLHLSGIRVFDAQALSNPYCSGIPSLTAVWGMIHSYQRKLNEALGTNVRFTSFSWFIRNYSAVAGKKLPELSLQGAQQSRLKRPGIIDGKYCDLVFDLIIHIDGYEDDLQAVDSKPDILKAHFPSNFAGGVMHQPELNSNINWCCLYSNENQLFEKLRRLPLSGCWVMPTEHKIQDLDELLLLLNSDSKLSPSMMGYMLLTEPMARVGSLERLHCYAEPAIGVVKYEAATSVRLKGIGNYFNSAFWMLDAQEKFMLMKKV

SEQ ID NO: 302 - protein, 732 aa, synthetic construct
MPKKKRKVGSGDYKDDDDKDYKDDDDKDYKDDDDKGSGHLKELLEITDTTERDRSLRRAFSPYTAMIDITGSEAVALIILLNLTYRKNQVDDLLDKKLAKQALKSEDHINKCIKEIAWFHTHNLKYPDIRVSKQNLAVEPPTLHSYVLSSANYPKAYGWSHNSAKVNFAKLFVSYFKWQNQVSWLAQVLATNSDNWKSAFTSLGLSVKAFKSLCVTVKNSLPEEAIPDSVDRYSRQIRMPYHDGYLAVTPVISHVVQSKIQQAAIDKRARFSNVEFTRPAAVSMLAASLGGVINVLNYPPYIRSKYHGLSNSRAFKLNNGQTVFNVEALLKPELIKALEGIIFSNNALALKQRRQQKVKNIKELRNTLLEWFSPVFEWRLDAIENGYDLEQLESASERLEYKILSLPDNELPSLTIPLFRLLNEMLGGVSMTQRYAFHPKLMSPLKAALQWLLVNLTDQKHVLIEEDDEHYRYLHLSGIRVFDAQALSNPYCSGIPSLTAVWGMIHSYQRKLNEALGTNVRFTSFSWFIRNYSAVAGKKLPELSLQGAQQSRLKRPGIIDGKYCDLVFDLIIHIDGYEDDLQAVDSKPDILKAHFPSNFAGGVMHQPELNSNINWCCLYSNENQLFEKLRRLPLSGCWVMPTEHKIQDLDELLLLLNSDSKLSPSMMGYMLLTEPMARVGSLERLHCYAEPAIGVVKYEAATSVRLKGIGNYFNSAFWMLDAQEKFMLMKKV

SEQ ID NO: 303 - protein, 349 aa, Pseudoalteromonas sp.
MELCNILKYDRSLYPGKAVFFYKTADSDFVPLEADINKIRGPKSGFTEAFTPQFSPKNISPQDLTHNNILTLEECYVPPNVEHIFCRFSLRVQANSLVPSGCSDPEVFSLLKELAETFKECGGYKELAVRYCRNILIGTWLWRNQNTGNTQIEIKTSKGSCYLIDNTRKLAWESKWASDDLKVLEELSNEIESALTDPNVFWSADITAKIEASFCQEIYPSQILNDKVKQGEASKQFVKAKCADGRYAVSFNSVKIGAALQSIDDWWDEDASKRLRVHEFGADKEIGVARRPPDSEQNFYSIFKNTEWYLSALKNCITNKNEKIDPAIYYLFSVLIKGGMFQKKAEAKK

SEQ ID NO: 304 - protein, 359 aa, synthetic construct
MPKKKRKVGSGELCNILKYDRSLYPGKAVFFYKTADSDFVPLEADINKIRGPKSGFTEAFTPQFSPKNISPQDLTHNNILTLEECYVPPNVEHIFCRFSLRVQANSLVPSGCSDPEVFSLLKELAETFKECGGYKELAVRYCRNILIGTWLWRNQNTGNTQIEIKTSKGSCYLIDNTRKLAWESKWASDDLKVLEELSNEIESALTDPNVFWSADITAKIEASFCQEIYPSQILNDKVKQGEASKQFVKAKCADGRYAVSFNSVKIGAALQSIDDWWDEDASKRLRVHEFGADKEIGVARRPPDSEQNFYSIFKNTEWYLSALKNCITNKNEKIDPAIYYLFSVLIKGGMFQKKAEAKK

SEQ ID NO: 305 - protein, 203 aa, Pseudoalteromonas sp.
MQRYYFTVHFLPKQANLALLTGRCISIMHGFILKHNIEGMGVTFPAWSDSSIGNEIAFVYTDKEILNTLKDQAYFVDMQDCGFFKVSQVLAVPDSCEEVRFIRNQAVAKIFTGESRRRLKRLQKRALARGEDFNPKKIEAPREIDIFHRVAMTSKSSQEDYILHIQKQDVDCQAEPYFSNYGLASNEKFKGTVPDLSPSIDRN

SEQ ID NO: 306 - protein, 213 aa, synthetic construct
MPKKKRKVGSGQRYYFTVHFLPKQANLALLTGRCISIMHGFILKHNIEGMGVTFPAWSDSSIGNEIAFVYTDKEILNTLKDQAYFVDMQDCGFFKVSQVLAVPDSCEEVRFIRNQAVAKIFTGESRRRLKRLQKRALARGEDFNPKKIEAPREIDIFHRVAMTSKSSQEDYILHIQKQDVDCQAEPYFSNYGLASNEKFKGTVPDLSPSIDRN

SEQ ID NO: 307 - protein, 698 aa, Vibrio parahaemolyticus
MIKLGDVLAIEEDEVKQATLKKVFMPYSENIDIDGREREALTVLINLSSHHKGSKCTDWLDIDRAKSYLSQEANVDLSLAEIKWFHTHNLKYPDCRVSAQRIIAEPLPAEDAFISSSGLPPSLGWAHNSASYRHTIWLLSSFCWQSRTFSIVSLIQQQNPVWLDLLQEFGLSVKSLNLISEEIELQLLSTAFPTEVNTYSKQLRFPWNGDYLSVTPVVSHAMQSELEHRQRSEDSHLKFVTMLLPNSASIGNLCGSVGGYMKVLNYPLDISPKVNRASSEQTLGASRQRNGRCFDDYQITNIRICEILNRLVGAEPLKTHKQRVKARKDQSKILRKQIALWMLPLIELRDRMVNDERERTMHGDQLIHDFLFLPERELSSLATSLNQKLHLVLQGNKFTRKFAYHPRLMQLIKAQIVWILDVLSKPQQQEGGCGAEEQYIYLSSLRVQDALAVSSPYLCGVPSLTAIWGFVHQYQRDFNTLTNGDAFYDFTGFAFYVRSQNIIATAKLTEPCSLAKARTLSNAKRSTIRGDRLTDLEIDLVIRVQSRGRLSDCSSELKNALPVSFAGGSVFQPRISSKIDWLRTFCSRSSLLHILKGLPAYGSWLYPSERQPESFDELELMLLENENYLPVSNGYHLLEVPTQRKNSLTDLHAYVENTLSVANQVNPIEMRFSGRAPFFEQAFWSLECRPTTILIKKL

SEQ ID NO: 308 - protein, 735 aa, synthetic construct
MPKKKRKVGSGDYKDDDDKDYKDDDDKDYKDDDDKGSGIKLGDVLAIEEDEVKQATLKKVFMPYSENIDIDGREREALTVLINLSSHHKGSKCTDWLDIDRAKSYLSQEANVDLSLAEIKWFHTHNLKYPDCRVSAQRIIAEPLPAEDAFISSSGLPPSLGWAHNSASYRHTIWLLSSFCWQSRTFSIVSLIQQQNPVWLDLLQEFGLSVKSLNLISEEIELQLLSTAFPTEVNTYSKQLRFPWNGDYLSVTPVVSHAMQSELEHRQRSEDSHLKFVTMLLPNSASIGNLCGSVGGYMKVLNYPLDISPKVNRASSEQTLGASRQRNGRCFDDYQITNIRICEILNRLVGAEPLKTHKQRVKARKDQSKILRKQIALWMLPLIELRDRMVNDERERTMHGDQLIHDFLFLPERELSSLATSLNQKLHLVLQGNKFTRKFAYHPRLMQLIKAQIVWILDVLSKPQQQEGGCGAEEQYIYLSSLRVQDALAVSSPYLCGVPSLTAIWGFVHQYQRDFNTLTNGDAFYDFTGFAFYVRSQNIIATAKLTEPCSLAKARTLSNAKRSTIRGDRLTDLEIDLVIRVQSRGRLSDCSSELKNALPVSFAGGSVFQPRISSKIDWLRTFCSRSSLLHILKGLPAYGSWLYPSERQPESFDELELMLLENENYLPVSNGYHLLEVPTQRKNSLTDLHAYVENTLSVANQVNPIEMRFSGRAPFFEQAFWSLECRPTTILIKKL

SEQ ID NO: 309 - protein, 345 aa, Vibrio parahaemolyticus
MELCSQLNYVRSLSAGKACFYYLTPSGDMCPLSIDKTRLRAPKGGYSEAYRGSQFHQKNVAPQDLAYANPQFIEECYVPPSTDEIVCEFSLRVKANSLHPEVCNDDSVREQLALLAATYKNLNGYQELAYRYAKNILLGTWLWRNRECRGVAIEVTTSDGEIILISDATRLSWYGHWDEKSTESLERLTSYLSRALSDNAQYFYMDVKAVLAVGRGDEVYPSQEFLDDKQEGVPTKQLAKVRLDDGRETAAFHAQKIGAALQSIDDWWHEEADKPLRVNEYGADREYVIARRHTQSGNDFYQLIRRTEAWTEEMEKLKSIPNDVHFIMSVLIKGGLFNSSKSTAK

SEQ ID NO: 310 - protein, 355 aa, synthetic construct
MPKKKRKVGSGELCSQLNYVRSLSAGKACFYYLTPSGDMCPLSIDKTRLRAPKGGYSEAYRGSQFHQKNVAPQDLAYANPQFIEECYVPPSTDEIVCEFSLRVKANSLHPEVCNDDSVREQLALLAATYKNLNGYQELAYRYAKNILLGTWLWRNRECRGVAIEVTTSDGEIILISDATRLSWYGHWDEKSTESLERLTSYLSRALSDNAQYFYMDVKAVLAVGRGDEVYPSQEFLDDKQEGVPTKQLAKVRLDDGRETAAFHAQKIGAALQSIDDWWHEEADKPLRVNEYGADREYVIARRHTQSGNDFYQLIRRTEAWTEEMEKLKSIPNDVHFIMSVLIKGGLFNSSKSTAK

SEQ ID NO: 311 - protein, 200 aa, Vibrio parahaemolyticus
MSKRYYFSIRYIPLHADFGLLAGRCIQQMHMFIVNNPQVKNKVGVCFPRWNVTNIGDTIAFVMDDKEMLSGLSFQPYFSMMVKEGVFEVSRVCEVPVDSPEVRFVRNQIIGKSFVASKQRRMKRSMLRADLSATEHTPIAKEERVVDHFHRVPISSASSGQEYLLHIQKEFVESREQANFNSYGLATNQEKRGTVPDLSI

SEQ ID NO: 312 - protein, 210 aa, synthetic construct
MPKKKRKVGSGSKRYYFSIRYIPLHADFGLLAGRCIQQMHMFIVNNPQVKNKVGVCFPRWNVTNIGDTIAFVMDDKEMLSGLSFQPYFSMMVKEGVFEVSRVCEVPVDSPEVRFVRNQIIGKSFVASKQRRMKRSMLRADLSATEHTPIAKEERVVDHFHRVPISSASSGQEYLLHIQKEFVESREQANFNSYGLATNQEKRGTVPDLSI

SEQ ID NO: 313 - protein, 644 aa, Aliiglaciecola sp.
MASNEITSLLNIENHTDRNVAWKKALSPITPPLDVTGNEKLACVVLANLTWKLSLINNVFDSNDARAKLRDKNWIQRCIKTFRYRHTHNLKYPDYRAKGAIRLSPIGVIPKGCFSSSKLISSRLGWSQNSADINYATFLCADFVWQGELLTLGEAIIGENISFTKSLIESGMFKKDLKLIRNELSQIPINQTESEYLSHQLTNLRFPKHSDGYVCLTPVPSHIVQVAIHSWSVSNFRQSETMYCPRSSSVGSLPACVGGKIKVLKSLPKGLNSKHTKDTQKSSWLTAENLAILHSLSSSRDWLLPENKKKKRYKELVAKLGAMLVRWMSFNRKSLEQLLESEFPSKQITQLFHADLSRLKSTDDIAYNPTFIKIVEQEFKIILENEKEDYPLVIPQQKHTHLVLPGLRVSNANAESCAYLVGLPSMIGIFGFIHNLQRQLDSRFGLSAGFEQFAICMHEYSFHKRGLTKEQVQISKKQLRSPAIIDSRQCDFALSLVIKTSAILQREEVLAALPQKICGGAVHIPLSELEGINTHHSFESAVNAIPVKNGKWITPSFNSLSTTNFIDFLDKTSVSYNLNIACVGYHYLETPFKKNSASDDPVHAFAEPILAGVQLNCIASFGNIERFFWHYSETSTSLYLGSKI

SEQ ID NO: 314 - protein, 681 aa, synthetic construct
MPKKKRKVGSGDYKDDDDKDYKDDDDKDYKDDDDKGSGASNEITSLLNIENHTDRNVAWKKALSPITPPLDVTGNEKLACVVLANLTWKLSLINNVFDSNDARAKLRDKNWIQRCIKTFRYRHTHNLKYPDYRAKGAIRLSPIGVIPKGCFSSSKLISSRLGWSQNSADINYATFLCADFVWQGELLTLGEAIIGENISFTKSLIESGMFKKDLKLIRNELSQIPINQTESEYLSHQLTNLRFPKHSDGYVCLTPVPSHIVQVAIHSWSVSNFRQSETMYCPRSSSVGSLPACVGGKIKVLKSLPKGLNSKHTKDTQKSSWLTAENLAILHSLSSSRDWLLPENKKKKRYKELVAKLGAMLVRWMSFNRKSLEQLLESEFPSKQITQLFHADLSRLKSTDDIAYNPTFIKIVEQEFKIILENEKEDYPLVIPQQKHTHLVLPGLRVSNANAESCAYLVGLPSMIGIFGFIHNLQRQLDSRFGLSAGFEQFAICMHEYSFHKRGLTKEQVQISKKQLRSPAIIDSRQCDFALSLVIKTSAILQREEVLAALPQKICGGAVHIPLSELEGINTHHSFESAVNAIPVKNGKWITPSFNSLSTTNFIDFLDKTSVSYNLNIACVGYHYLETPFKKNSASDDPVHAFAEPILAGVQLNCIASFGNIERFFWHYSETSTSLYLGSKI

SEQ ID NO: 315 - protein, 343 aa, Aliiglaciecola sp.
MRLPNRLSYQRSISPGIAVFYSVDEQGNQKPLEINTVKILGQKGGPSEAFANDMSLKKGVDNKKLAEGNPHTIDYCYAPADAKHTLCKFSLNVDASSIEPRACNDDGVRSLLTNFAAEYRKLGGYRYLAERYLNNVLSGNWLWRNQRTLDTTIKIQSSGGLQCSIKGVNRKRFEPNWIDEITEFDGLVNEFENALVDPKKYLFLEVTAELSLPLASEIYPSQAFVEQANKLERSRTYQNTIVEGKRTAIIGAYKIGAAIASIDDWFEGADIPVRVGSFAVDRDRATVYRHPESKKDFYTLLSGLEQLNSRLKSKKKMKSSELNDAHFIAANLVKGGLFSLGSK

SEQ ID NO: 316 - protein, 353 aa, synthetic construct
MPKKKRKVGSGRLPNRLSYQRSISPGIAVFYSVDEQGNQKPLEINTVKILGQKGGPSEAFANDMSLKKGVDNKKLAEGNPHTIDYCYAPADAKHTLCKFSLNVDASSIEPRACNDDGVRSLLTNFAAEYRKLGGYRYLAERYLNNVLSGNWLWRNQRTLDTTIKIQSSGGLQCSIKGVNRKRFEPNWIDEITEFDGLVNEFENALVDPKKYLFLEVTAELSLPLASEIYPSQAFVEQANKLERSRTYQNTIVEGKRTAIIGAYKIGAAIASIDDWFEGADIPVRVGSFAVDRDRATVYRHPESKKDFYTLLSGLEQLNSRLKSKKKMKSSELNDAHFIAANLVKGGLFSLGSK

SEQ ID NO: 317 - protein, 201 aa, Aliiglaciecola sp.
MASRYYRKITFIPADSNHNFLIGKCLKVLHGVNCRHRLNSIGVTFPDWSDESPGNSIAFVSVDSACIDLLIDQHYYQQMQDLEYFEISALKPVPENGSEEIMFSRNQAVDELTPAGVRRKLRRCARRAKQRGENYNAAYLSSSEKVFPHFHKIPMNSKSSDRNFSLNIQLEMAQNVTYGNYTSYGLSNKSSRKASVPKNLD

SEQ ID NO: 318 - protein, 211 aa, synthetic construct
MPKKKRKVGSGASRYYRKITFIPADSNHNFLIGKCLKVLHGVNCRHRLNSIGVTFPDWSDESPGNSIAFVSVDSACIDLLIDQHYYQQMQDLEYFEISALKPVPENGSEEIMFSRNQAVDELTPAGVRRKLRRCARRAKQRGENYNAAYLSSSEKVFPHFHKIPMNSKSSDRNFSLNIQLEMAQNVTYGNYTSYGLSNKSSRKASVPKNLD

SEQ ID NO: 319 - protein, 675 aa, Oceanospirillum linum
MLKDLLEKKEGTRAEFNHKVKRCFEPYTPLIEADGAELECVIILANLASRAAETLDDRASAKSSLTTDNFWKKVLQSAQQLHTHNLKFPDARVHYKNRIRVINPQDQFPVLGWSGNSSDYNFARFLNSAFQWQNERHTLLTVLLDDLPAWRNAFSRLGVFKAQWHQLRQQLKQIFQTSTFPDTVDIYSPQLRLPWRGRHLIAITPVVNHTLQLKIQSSAKELPSIKISYPRPSAIGQLCGALGGNLRYLHYHPIPKGLIGFQQQLSVDRESLLSQRSLSGKHPESVYKSLIDRRINASLRLARLARRDALRQFDLILENWLKALMDVRQYFLETGCLHYKNLNRVEESFVRDEASSNDLRKYLNTSFHKSLRLNPYTQDFAYHPGLTATLNQRLKQLLHQENAPSAAEELPEMGYASLHNVSVTDGNALNNPYCAGMPSMTGLWGFCKNLEMQLKESGFAVSVQRVALMCHEFSANRSTLIPEPSRPSPQKGSQTVKRSGLLPQFTFSGQFSVVIEYRKSAGRLSELTTDDLRNHLPDRLWGGSLMLQESANNHGIHLTDEFDPLYRKLIRQFRRGVWLVPDSSEVIEQNSLFDLLLEDKKRAPLLTGFKALEEPKIREGALCGLHFYAEPAIGICRRETMFRLTKSPDYFLNKAFWGLTPATNNDESIHLIRRV

SEQ ID NO: 320 - protein, 712 aa, synthetic construct
MPKKKRKVGSGDYKDDDDKDYKDDDDKDYKDDDDKGSGLKDLLEKKEGTRAEFNHKVKRCFEPYTPLIEADGAELECVIILANLASRAAETLDDRASAKSSLTTDNFWKKVLQSAQQLHTHNLKFPDARVHYKNRIRVINPQDQFPVLGWSGNSSDYNFARFLNSAFQWQNERHTLLTVLLDDLPAWRNAFSRLGVFKAQWHQLRQQLKQIFQTSTFPDTVDIYSPQLRLPWRGRHLIAITPVVNHTLQLKIQSSAKELPSIKISYPRPSAIGQLCGALGGNLRYLHYHPIPKGLIGFQQQLSVDRESLLSQRSLSGKHPESVYKSLIDRRINASLRLARLARRDALRQFDLILENWLKALMDVRQYFLETGCLHYKNLNRVEESFVRDEASSNDLRKYLNTSFHKSLRLNPYTQDFAYHPGLTATLNQRLKQLLHQENAPSAAEELPEMGYASLHNVSVTDGNALNNPYCAGMPSMTGLWGFCKNLEMQLKESGFAVSVQRVALMCHEFSANRSTLIPEPSRPSPQKGSQTVKRSGLLPQFTFSGQFSVVIEYRKSAGRLSELTTDDLRNHLPDRLWGGSLMLQESANNHGIHLTDEFDPLYRKLIRQFRRGVWLVPDSSEVIEQNSLFDLLLEDKKRAPLLTGFKALEEPKIREGALCGLHFYAEPAIGICRRETMFRLTKSPDYFLNKAFWGLTPATNNDESIHLIRRV

SEQ ID NO: 321 - protein, 355 aa, Oceanospirillum linum
MNLPNQLTYKRSLHPGPAVFFYEDAEEKQHPLTIERTKIRGSKSGFAEAYQVKKDKAAESGINISLKPDATTQKLSSGNPHTIDTCYLPPEAETLICKFSLRIAANSLKPDTCSDAECWNSLTNFTALYKKAGGYFELAERYAKNILSGAWLWRNRDTAAFEITVETSEGNTYTLPNAHLQFPDIPWKKDTAKILKGLATEIETALASPRYYWSAEITARLKPGFCAEIFPSQCFTDPSDSDASKVLATINYQGAKTACMTADKVNAAIQRVDNWYSDDPNASPLRVNEYGSDSHRNIACRHPSTQLDFYTLLQGIDEQISVLEKAKSLKDIPASTHYITSVLTKGGMFQGGKAK

SEQ ID NO: 322 - protein, 365 aa, synthetic construct
MPKKKRKVGSGNLPNQLTYKRSLHPGPAVFFYEDAEEKQHPLTIERTKIRGSKSGFAEAYQVKKDKAAESGINISLKPDATTQKLSSGNPHTIDTCYLPPEAETLICKFSLRIAANSLKPDTCSDAECWNSLTNFTALYKKAGGYFELAERYAKNILSGAWLWRNRDTAAFEITVETSEGNTYTLPNAHLQFPDIPWKKDTAKILKGLATEIETALASPRYYWSAEITARLKPGFCAEIFPSQCFTDPSDSDASKVLATINYQGAKTACMTADKVNAAIQRVDNWYSDDPNASPLRVNEYGSDSHRNIACRHPSTQLDFYTLLQGIDEQISVLEKAKSLKDIPASTHYITSVLTKGGMFQGGKAK

SEQ ID NO: 323 - protein, 200 aa, Oceanospirillum linum
MKWHYFIIRYIPSDADEFLLAGRCILALHHFLYRNKANSIGIHFPDWSDRSVGKRIAFMSENEDLLTWFKKERYFLTMAENDLFEMTEIVQTSLTDKKGVAFVRNQKAGKLTSASKARRIRRAKRRAEARGEVYKSRNQESDRELDHFHSIHMESTSTGKAFTLFVGKVEEPGTGLSQKEFNSYGLSSQNQQMVLLPIIS

SEQ ID NO: 324 - protein, 210 aa, synthetic construct
MPKKKRKVGSGKWHYFIIRYIPSDADEFLLAGRCILALHHFLYRNKANSIGIHFPDWSDRSVGKRIAFMSENEDLLTWFKKERYFLTMAENDLFEMTEIVQTSLTDKKGVAFVRNQKAGKLTSASKARRIRRAKRRAEARGEVYKSRNQESDRELDHFHSIHMESTSTGKAFTLFVGKVEEPGTGLSQKEFNSYGLSSQNQQMVLLPIIS

SEQ ID NO: 325 - protein, 640 aa, Vibrio anguillarum
MQTLKELIESTPDDLTTVLKRAFRPLTPHIAIDGNELDALTILVNLTDKTDDQKDLLDRAKCKQKLRDEKWWASCLNCVNYRQSHNPKFPDIRSEGIIRTEALGELPSFLLSSSKIPPYHWSYAHDSKYVNKSALLTNEFCWNGVISCLAELLKNVDHPLWKTLTKLGCYQKTRKAMAKKLASIAHITISMPLAPNYLTQISLPNSDTSYISLSPVASLSMQSHFYQGLQDEYRHASTTRFSRATNMGVTAMTCGGAFRMLKSNTKFSITPHHRLNSKRSWLTSENVQSLKQYQRLNKRLIPENARKALRRKYKIEIQNMVSVWLAMQDHTLDSIILVQHLNHDLSCLGATKRFAYNPVMTKLFTELLKRALSNSLNDSTHYSNGSFLVLPNIRVCGATALSSPVTVGIPSLTAFFGFVHAFERKLNRLNPTFRVESFAICVHQLHVEKRGLTAEFVEKGNGTISAPATRDDWQCDVVFSLILNTNFAQRIDQSTLITLLPKRFARGSAKIAIDDFKHINSFSTLEAAIQSLPIEAGRWLSLYAQPNNNLGDLLAAMKEDHQLMASCVGYHLLEEPKDKPNSLRSYKHAFAECIIGLINSITFSSETDANTIFWSLNNHQNYLVVQPRIINDETTDKSSL

SEQ ID NO: 326 - protein, 677 aa, synthetic construct
MPKKKRKVGSGDYKDDDDKDYKDDDDKDYKDDDDKGSGQTLKELIESTPDDLTTVLKRAFRPLTPHIAIDGNELDALTILVNLTDKTDDQKDLLDRAKCKQKLRDEKWWASCLNCVNYRQSHNPKFPDIRSEGIIRTEALGELPSFLLSSSKIPPYHWSYAHDSKYVNKSALLTNEFCWNGVISCLAELLKNVDHPLWKTLTKLGCYQKTRKAMAKKLASIAHITISMPLAPNYLTQISLPNSDTSYISLSPVASLSMQSHFYQGLQDEYRHASTTRFSRATNMGVTAMTCGGAFRMLKSNTKFSITPHHRLNSKRSWLTSENVQSLKQYQRLNKRLIPENARKALRRKYKIEIQNMVSVWLAMQDHTLDSIILVQHLNHDLSCLGATKRFAYNPVMTKLFTELLKRALSNSLNDSTHYSNGSFLVLPNIRVCGATALSSPVTVGIPSLTAFFGFVHAFERKLNRLNPTFRVESFAICVHQLHVEKRGLTAEFVEKGNGTISAPATRDDWQCDVVFSLILNTNFAQRIDQSTLITLLPKRFARGSAKIAIDDFKHINSFSTLEAAIQSLPIEAGRWLSLYAQPNNNLGDLLAAMKEDHQLMASCVGYHLLEEPKDKPNSLRSYKHAFAECIIGLINSITFSSETDANTIFWSLNNHQNYLVVQPRIINDETTDKSSL

SEQ ID NO: 327 - protein, 352 aa, Vibrio anguillarum
MKLPTSLAYERSIDPSDVCFFVVWPDDKKTPLTYTSRTLLGQMETASLAYDASGQPIKSATAEALAQGNPHQVDICRVPFGASHVECCFSVSFSCELRKPYKCNSSSVKQTLVQLIELYEMKIGWTELATRYLINICNGAWLWENTRKAYCWNIELAPWPWNGNKVKFEDIRSSYRSRQDFESHKDWSAITKMIKTAFSSSNGLAIFEVKATLHLPTNAMVRPSQAFTEKESGSKSKSKSQNSRVFQSTTIDGERSPILGAFKTGAAIATIDDWYPGATESLRVGRFGVHREDVTCYRHPSTGKDLFSILQQAEHYIEVLNANKTPDQETINDMHFLLANLIKGGMFQHKGD

SEQ ID NO: 328 - protein, 362 aa, synthetic construct
MPKKKRKVGSGKLPTSLAYERSIDPSDVCFFVVWPDDKKTPLTYTSRTLLGQMETASLAYDASGQPIKSATAEALAQGNPHQVDICRVPFGASHVECCFSVSFSCELRKPYKCNSSSVKQTLVQLIELYEMKIGWTELATRYLINICNGAWLWENTRKAYCWNIELAPWPWNGNKVKFEDIRSSYRSRQDFESHKDWSAITKMIKTAFSSSNGLAIFEVKATLHLPTNAMVRPSQAFTEKESGSKSKSKSQNSRVFQSTTIDGERSPILGAFKTGAAIATIDDWYPGATESLRVGRFGVHREDVTCYRHPSTGKDLFSILQQAEHYIEVLNANKTPDQETINDMHFLLANLIKGGMFQHKGD

SEQ ID NO: 329 - protein, 199 aa, Vibrio anguillarum
MNWYNKTITFLPERCDNEVLAAKCLSTLHAFNYKYDTRSIGISFPGWCEDTVGKKLTFISTSKVELDLLLKHQYFIQMRKLSYFDISATAQIPDGCEYVSFVRNQSIDKSSAAGQTRKLRRLEKRATARGESFNPALIKQRESIILPHYHSLEIDSQSKKCIFPLNIQMKSEQSFEGDSIFSSYGLSNTDNSFQPVPLI

SEQ ID NO: 330 - protein, 209 aa, synthetic construct
MPKKKRKVGSGNWYNKTITFLPERCDNEVLAAKCLSTLHAFNYKYDTRSIGISFPGWCEDTVGKKLTFISTSKVELDLLLKHQYFIQMRKLSYFDISATAQIPDGCEYVSFVRNQSIDKSSAAGQTRKLRRLEKRATARGESFNPALIKQRESIILPHYHSLEIDSQSKKCIFPLNIQMKSEQSFEGDSIFSSYGLSNTDNSFQPVPLI

SEQ ID NO: 331 - protein, 701 aa, Halomonas sp.
MRQAAIIIIYQRGNVMSLSTLLELDEPNRSEAIRKAFAPYTPLIEVSEDVSVAILVLLNLSHKRKYAPDLLNKKRAIETLKDWQHMESCAQEVQWVHSHNLKHPDTRVAHQRLLVKAEKPSDSIVSSYNSVSRLGWSHNSAAVNKAKLFGANFIFKGVVCCLAAIVLDNNKQWRKEFMNLGMSGDQWAYLQSLFDNYFTKNLSPSYVDRHSVQVTFLYKGKDVSITPVTSHSLLADIQIARRNKCGDLATIKHWHSSSVGDLASSLGGNISALSYPPRLLACSQNKENENSSGIFFVDFHHSSLRSKSFILACNEIVESKSLLTGKKRRDHRRSAIKLLRQSLSEWLSPVSYWRSVGGEVLSERQNNSACLLISAPNEDLLEILPEVNKELHSILVRYPQTQSFAYHPELLIPFKAQLKSLLIGMKIKDDEPMAEEPYHYLHLTNLHVFDAQALSCPYLVGLPSLLAVWGTVYNYQLRLRNILKRNIVFEGVAWFLRQYESSSGAKIPAPYLPPMKPGETPKRPGLIDMRFCDLRMDLVICYRLEDGDDTPLGNDELTMLQSAFPGRFAGGTMQPPPLYEELQWCQLHGDANSLLAAISLLPDEGRWVVDSEKQVQSIDSLVAWLTKHPNHLPAMSGYQLLEEPCYRSGSHRELHAYAEPLVGLTETLSPASVRLNGKADFLKNAFWRLKSQNLTMLMKKA

SEQ ID NO: 332 - protein, 738 aa, synthetic construct
MPKKKRKVGSGDYKDDDDKDYKDDDDKDYKDDDDKGSGRQAAIIIIYQRGNVMSLSTLLELDEPNRSEAIRKAFAPYTPLIEVSEDVSVAILVLLNLSHKRKYAPDLLNKKRAIETLKDWQHMESCAQEVQWVHSHNLKHPDTRVAHQRLLVKAEKPSDSIVSSYNSVSRLGWSHNSAAVNKAKLFGANFIFKGVVCCLAAIVLDNNKQWRKEFMNLGMSGDQWAYLQSLFDNYFTKNLSPSYVDRHSVQVTFLYKGKDVSITPVTSHSLLADIQIARRNKCGDLATIKHWHSSSVGDLASSLGGNISALSYPPRLLACSQNKENENSSGIFFVDFHHSSLRSKSFILACNEIVESKSLLTGKKRRDHRRSAIKLLRQSLSEWLSPVSYWRSVGGEVLSERQNNSACLLISAPNEDLLEILPEVNKELHSILVRYPQTQSFAYHPELLIPFKAQLKSLLIGMKIKDDEPMAEEPYHYLHLTNLHVFDAQALSCPYLVGLPSLLAVWGTVYNYQLRLRNILKRNIVFEGVAWFLRQYESSSGAKIPAPYLPPMKPGETPKRPGLIDMRFCDLRMDLVICYRLEDGDDTPLGNDELTMLQSAFPGRFAGGTMQPPPLYEELQWCQLHGDANSLLAAISLLPDEGRWVVDSEKQVQSIDSLVAWLTKHPNHLPAMSGYQLLEEPCYRSGSHRELHAYAEPLVGLTETLSPASVRLNGKADFLKNAFWRLKSQNLTMLMKKA

SEQ ID NO: 333 - protein, 349 aa, Halomonas sp.
MMNSFRHLSYERSLNPGKAVFYYRTDSSEFEPLQAEVTRFRGPKATFSDGYMASGTARAKETSDLGFSNPIMLETCYVPPLVDTLYCRFSLRIIANSLEPNICDNAEATKALKEFSDTYRNLGGYQELATRYAKNILSAEWLWKNKVSRGIAVVVSTSNLKNYCVKDAQYKEWGSSWEGDELKSLEGLAVEFEEALSCPQKFLFADVTAKIKTEFCQEIFPSQLFVEKDDRGNGSASRKFMKSTMNDGRQAVSFGAYKVGAAIQKIDDWWLDEGAEYPLRVSEYGADRSRVLAMREPVTKKDFYSLLNEIINITEEMIKTRQASPNAHYVMSVLVKGGMFQKGIKKGEK

SEQ ID NO: 334 - protein, 359 aa, synthetic construct
MPKKKRKVGSGMNSFRHLSYERSLNPGKAVFYYRTDSSEFEPLQAEVTRFRGPKATFSDGYMASGTARAKETSDLGFSNPIMLETCYVPPLVDTLYCRFSLRIIANSLEPNICDNAEATKALKEFSDTYRNLGGYQELATRYAKNILSAEWLWKNKVSRGIAVVVSTSNLKNYCVKDAQYKEWGSSWEGDELKSLEGLAVEFEEALSCPQKFLFADVTAKIKTEFCQEIFPSQLFVEKDDRGNGSASRKFMKSTMNDGRQAVSFGAYKVGAAIQKIDDWWLDEGAEYPLRVSEYGADRSRVLAMREPVTKKDFYSLLNEIINITEEMIKTRQASPNAHYVMSVLVKGGMFQKGIKKGEK

SEQ ID NO: 335 - protein, 202 aa, Halomonas sp.
MRYFFYIKYLMPSANHAFLAGRCIACLHGFISGPKITNSGIGVSFPSWATGTVGDSIAFVSKDINSLSYLSSARYFKNMADEGFIDVSDIKMVPETLEEVRFIRNQHIAKSFPGEIKRRLIRSKNRAEKRGETFMPSSAVSDRFVDQCHVIPIDSRSSGQRFPLYVQLEALGEESKYDNYNSYGLATQHTHSGTVPNLKQIT

SEQ ID NO: 336 - protein, 212 aa, synthetic construct
MPKKKRKVGSGRYFFYIKYLMPSANHAFLAGRCIACLHGFISGPKITNSGIGVSFPSWATGTVGDSIAFVSKDINSLSYLSSARYFKNMADEGFIDVSDIKMVPETLEEVRFIRNQHIAKSFPGEIKRRLIRSKNRAEKRGETFMPSSAVSDRFVDQCHVIPIDSRSSGQRFPLYVQLEALGEESKYDNYNSYGLATQHTHSGTVPNLKQIT

SEQ ID NO: 337 - protein, 699 aa, Vibrio sp.
MKLSDVLRIEDEVLKQTTFKKVFMPYSEDIEIDGCEKEALIILLNLSYYPKGTKHINWLDDERALDYLTEQDNLTASLAEVQWFHTHNLKYPDCRVSKQKIIGEPLPADDVFISSATLKPILGWAHNSAAYRYTIWLLNSFIWQSQPTNILTLIEQQNPIWLDLLRAFGLREKSLELLRTEIELQLSSQSFPRYVDSYSKQLRFPWNGDYLSVTPVVSHAMQRELEHRYRNAESHLKFVTLSFPNSASIGNLCGSVGGNMQVLNYPLDVPSSTNRSTLRKTLADSRLASGRYFDDFQLTNERICKVLSRLTGTETSTTHKRRIKSRKDQSRILRKQVALWMLPLIELRDRFDSDEREGVIEEHESLVQDFLTLSESDLPVLVSQFNQRLHYVFQENKFTRKFAYHPKLLQVVKSQIVWVLNKLSKPQEDEVSGQGEQYIYLSSLRVQDSLAMSCPYLCGVPSLTAIWGFVHHYQREFNRSINSDVFYEFAGFSIYVRSQSITVGAKLTEPNSVEKVRTLSNAKRPTIRTDRFADLEIDLVICVKSNGRLSDYRAALKSVLPLSLAGGSLFQPLISSKIDWLRTFDSQSSLFHALKGLPAYGRWLYPCELQPDSFDELESTLDQNSGCLPVSNGYHFLEIPIHRNNALTALHTYAENTLTVAKQVIPIEMRFAGSKQFFQEAFWSLECSSTTILVKKYKE

SEQ ID NO: 338 - protein, 736 aa, synthetic construct
MPKKKRKVGSGDYKDDDDKDYKDDDDKDYKDDDDKGSGKLSDVLRIEDEVLKQTTFKKVFMPYSEDIEIDGCEKEALIILLNLSYYPKGTKHINWLDDERALDYLTEQDNLTASLAEVQWFHTHNLKYPDCRVSKQKIIGEPLPADDVFISSATLKPILGWAHNSAAYRYTIWLLNSFIWQSQPTNILTLIEQQNPIWLDLLRAFGLREKSLELLRTEIELQLSSQSFPRYVDSYSKQLRFPWNGDYLSVTPVVSHAMQRELEHRYRNAESHLKFVTLSFPNSASIGNLCGSVGGNMQVLNYPLDVPSSTNRSTLRKTLADSRLASGRYFDDFQLTNERICKVLSRLTGTETSTTHKRRIKSRKDQSRILRKQVALWMLPLIELRDRFDSDEREGVIEEHESLVQDFLTLSESDLPVLVSQFNQRLHYVFQENKFTRKFAYHPKLLQVVKSQIVWVLNKLSKPQEDEVSGQGEQYIYLSSLRVQDSLAMSCPYLCGVPSLTAIWGFVHHYQREFNRSINSDVFYEFAGFSIYVRSQSITVGAKLTEPNSVEKVRTLSNAKRPTIRTDRFADLEIDLVICVKSNGRLSDYRAALKSVLPLSLAGGSLFQPLISSKIDWLRTFDSQSSLFHALKGLPAYGRWLYPCELQPDSFDELESTLDQNSGCLPVSNGYHFLEIPIHRNNALTALHTYAENTLTVAKQVIPIEMRFAGSKQFFQEAFWSLECSSTTILVKKYKE

SEQ ID NO: 339 - protein, 351 aa, Vibrio sp.
MELCRQLNYLRSISPGKAYFYYLASNGDRCPLAIDKTHIRAPKGGYAEAYQGSSFVKKNVAPQDLSYSNPQFIEECYVPPLTNEIICEFSLRIRANSLHPDVCSDEKVREQLMSLAKVYKELNGYQELAYRYAKNILLGSWLWRNKDCRGVTIQVMTSDGESIEVYDATKLSWYGHWDEQSTQSLEQLTSYLSRALSDRSQCFYMDVKAVMSVGRGDEVYPSQEFIDVKQEGIPTRQLAKVPLNYEQETAAFHAQKIGAALQSIDDWWHENADKPLRVNEYGADREYVIARRHSLLGNDFYQLIRRTEKWIEEMDKSKSIPNDVHFILSVLIKGGLFNCSKTKSKSKSKSK

SEQ ID NO: 340 - protein, 361 aa, synthetic construct
MPKKKRKVGSGELCRQLNYLRSISPGKAYFYYLASNGDRCPLAIDKTHIRAPKGGYAEAYQGSSFVKKNVAPQDLSYSNPQFIEECYVPPLTNEIICEFSLRIRANSLHPDVCSDEKVREQLMSLAKVYKELNGYQELAYRYAKNILLGSWLWRNKDCRGVTIQVMTSDGESIEVYDATKLSWYGHWDEQSTQSLEQLTSYLSRALSDRSQCFYMDVKAVMSVGRGDEVYPSQEFIDVKQEGIPTRQLAKVPLNYEQETAAFHAQKIGAALQSIDDWWHENADKPLRVNEYGADREYVIARRHSLLGNDFYQLIRRTEKWIEEMDKSKSIPNDVHFILSVLIKGGLFNCSKTKSKSKSKSK

SEQ ID NO: 341 - protein, 200 aa, Vibrio sp.
MERRYYFSIRYVPSYADFGLLAGRCIYQMHLFSVNNPEVKNKVGVCFPRWNSKDVGDMIAFVMEDKEALLGLAFQPYFSRMTKEGVFELSKVDEVPKSSSEVRFVRNQAIGKSFIASKKRRIKRSMTRAELLDFEHTPVAVEERVVEHYHRIPISSGSSGQDYILHIQKERVESRGQQDFSSYGLATKQEKRGTVPALYI

SEQ ID NO: 342 - protein, 210 aa, synthetic construct
MPKKKRKVGSGERRYYFSIRYVPSYADFGLLAGRCIYQMHLFSVNNPEVKNKVGVCFPRWNSKDVGDMIAFVMEDKEALLGLAFQPYFSRMTKEGVFELSKVDEVPKSSSEVRFVRNQAIGKSFIASKKRRIKRSMTRAELLDFEHTPVAVEERVVEHYHRIPISSGSSGQDYILHIQKERVESRGQQDFSSYGLATKQEKRGTVPALYI

SEQ ID NO: 343 - protein, 695 aa, Photobacterium aquae
MKKLCDVLQIEDNTEKQATLKKVFMPYSACIDIDGCEKEALTVLLNLSTHRKGSPCGDWLDIERAKSYLKDQADIDASLAEIKWFHTHNLKFPDCRVKEQRLIAKPLSTSESFISSVSLDQGLGWAHNSAVYRHTLWLLNSFNWQSESVNILSLVQEENPVWLELLQEFGLNIKQQDLLLKTIELQIPASTFPDSVSPYSKQLRFPWNNDYLSVTPVVSHAIQREIEVKARDKASKLSFVTSALPNSASIGNLCGSLGGYMKALNYPLDVKSVAEQTLAASRNKSGKYFDDFQVTNYKICQVLNRLIGAEPLKNQKQREKARKVQSKILRKQIALWMLPLIELRDIEDAEPHNQQLEHDDPLVKSFLSLPESEFPSLVHELNQRLHFVFQENKFTAKFAYHPKLIQVVKAQIVWVLEQLSKPSDHEDAAREQQYIYLSSLRVQDAVAMSSPYLCGAPSLTAIWGFMHHYQREFNKLVNSDSPFEFSRFAFYVRTENIQSTAKLTEPNSLAKSRTLSNAKRPTIRSERLADLEIDLVIRVDSDSRISDFLSELRAALPAAFAGGALYQPLILSQIDWLRTFSSKSELFHVLKGIPAYGSWLYPSEKQPTNFNELEHLITEDADNLPVSIGYHLLEHPTERENSITDCHAYAENALGIAKRLNPIEVRFSGRDHFFDNAFWALESTSATILIKNDRN

SEQ ID NO: 344 - protein, 732 aa, synthetic construct
MPKKKRKVGSGDYKDDDDKDYKDDDDKDYKDDDDKGSGKKLCDVLQIEDNTEKQATLKKVFMPYSACIDIDGCEKEALTVLLNLSTHRKGSPCGDWLDIERAKSYLKDQADIDASLAEIKWFHTHNLKFPDCRVKEQRLIAKPLSTSESFISSVSLDQGLGWAHNSAVYRHTLWLLNSFNWQSESVNILSLVQEENPVWLELLQEFGLNIKQQDLLLKTIELQIPASTFPDSVSPYSKQLRFPWNNDYLSVTPVVSHAIQREIEVKARDKASKLSFVTSALPNSASIGNLCGSLGGYMKALNYPLDVKSVAEQTLAASRNKSGKYFDDFQVTNYKICQVLNRLIGAEPLKNQKQREKARKVQSKILRKQIALWMLPLIELRDIEDAEPHNQQLEHDDPLVKSFLSLPESEFPSLVHELNQRLHFVFQENKFTAKFAYHPKLIQVVKAQIVWVLEQLSKPSDHEDAAREQQYIYLSSLRVQDAVAMSSPYLCGAPSLTAIWGFMHHYQREFNKLVNSDSPFEFSRFAFYVRTENIQSTAKLTEPNSLAKSRTLSNAKRPTIRSERLADLEIDLVIRVDSDSRISDFLSELRAALPAAFAGGALYQPLILSQIDWLRTFSSKSELFHVLKGIPAYGSWLYPSEKQPTNFNELEHLITEDADNLPVSIGYHLLEHPTERENSITDCHAYAENALGIAKRLNPIEVRFSGRDHFFDNAFWALESTSATILIKNDRN

SEQ ID NO: 345 - protein, 345 aa, Photobacterium aquae
MELCNQLNYVRSLSAGKAYFYHLSKGGEMCPLEIDRTRLRAPKGGYAEAYKGSKFVQKNVAPQDLAYANPQFIEECYVKPGVDDIYCAFSLRIKANSLGPDVCCDDEVRSKLSSLAKSYKELSGYSELAHRYAKNILLGTWLWRNRECRRLSIEVTTSDSETLIVENATKLTWYDHWDKDAAECLDKLTAYLTRALSDPTEYFYMDVKAKIAVGWGDEVYPSQEFLDNREDGVPTKQLATVELENGRETVAFHGQKVGAALQSIDDWWHEKADKPLRVNEYGADREYVIARRHVSLKNDFYQLLRNTENWIESMNTSNIIPNDVHFIMSVLVKGGLFNCSKSKSK

SEQ ID NO: 346 - protein, 355 aa, synthetic construct
MPKKKRKVGSGELCNQLNYVRSLSAGKAYFYHLSKGGEMCPLEIDRTRLRAPKGGYAEAYKGSKFVQKNVAPQDLAYANPQFIEECYVKPGVDDIYCAFSLRIKANSLGPDVCCDDEVRSKLSSLAKSYKELSGYSELAHRYAKNILLGTWLWRNRECRRLSIEVTTSDSETLIVENATKLTWYDHWDKDAAECLDKLTAYLTRALSDPTEYFYMDVKAKIAVGWGDEVYPSQEFLDNREDGVPTKQLATVELENGRETVAFHGQKVGAALQSIDDWWHEKADKPLRVNEYGADREYVIARRHVSLKNDFYQLLRNTENWIESMNTSNIIPNDVHFIMSVLVKGGLFNCSKSKSK

SEQ ID NO: 347 - protein, 201 aa, Photobacterium aquae
MTTRYYFTIQYIPTHADFGLLAGRCIYQMHKFMVNNPLAMNQIGVSFPMWEDGSVGNIIAFISEDKELMVGLLFQPYFSLMVKEGLFEISSVCEVPTDSPEVRFVRNQTIGKSFIGSKKRRIKRSMARAELSGAEYSLPVAVEERVVDHFHRVPISSGSSGHDYILHIQKEVASERSVANFNSYGLATNQEKRGTVPDLCI

SEQ ID NO: 348 - protein, 211 aa, synthetic construct
MPKKKRKVGSGTTRYYFTIQYIPTHADFGLLAGRCIYQMHKFMVNNPLAMNQIGVSFPMWEDGSVGNIIAFISEDKELMVGLLFQPYFSLMVKEGLFEISSVCEVPTDSPEVRFVRNQTIGKSFIGSKKRRIKRSMARAELSGAEYSLPVAVEERVVDHFHRVPISSGSSGHDYILHIQKEVASERSVANFNSYGLATNQEKRGTVPDLCI

SEQ ID NO: 349 - protein, 687 aa, Enterovibrio coralii
MKTLRDVLEDEEPDIALRKAFAAYSELVDVTGEETQTLIVLLNLTLKRDEVESLTSRKSARAVLKDEAHIDSCLEEVRWLHSHNLKYPDTRVQAQRILCGDLPLIAGVLGSANCERRLGWSHNSSQVNKAKLFCSGFIWEGSSTCLAESVIKNSDAWRRAFREFGLTKTKFEEWRLQLKQVMNTDHFPSEVSDYSKQVRFPWLSDYFAITPVVSSAVLAKIQQLRTQRLGHFRQIDHCHPASVGDFAASRGGGVTVLNYPLNIVWRNHVSLNQSRIRRVESDKSAFNSWALLNERFIGVLNSLIHLDEEPVLRRRRRRRVSLVRQLRRGIAEWLLPIMEWRDSLRDGADTLAAIRETERALLTEPLSDNTKLLKLVNQRFHTTLQDAGYRNTEYAYHPKLLEPVRNQLRWILDTLGNDQFGQRNTQFEVIHLENLRVFDALSLANPYLVGIPSLTALWGFIHAFDRKLKTLLGCEFTFESVAWHVRESSSVSGLKLPSPALERKRSDHLKRPGMIESKHCDLVMDLAIRVHSTEQFLQTRDELVDLIKAALPSRFAGGVIHPPSLYESRDWCSLRTTQSLHEHVSRLPATGRWIVPATTTPKSFENLCELVELNSDLKPAMLGYQLLEEPIERPNSVASLHAYAEPLIGLCDCKSSIDIRLKGEKYFNANFFWKMDTATSSILMRRA

SEQ ID NO: 350 - protein, 724 aa, synthetic construct
MPKKKRKVGSGDYKDDDDKDYKDDDDKDYKDDDDKGSGKTLRDVLEDEEPDIALRKAFAAYSELVDVTGEETQTLIVLLNLTLKRDEVESLTSRKSARAVLKDEAHIDSCLEEVRWLHSHNLKYPDTRVQAQRILCGDLPLIAGVLGSANCERRLGWSHNSSQVNKAKLFCSGFIWEGSSTCLAESVIKNSDAWRRAFREFGLTKTKFEEWRLQLKQVMNTDHFPSEVSDYSKQVRFPWLSDYFAITPVVSSAVLAKIQQLRTQRLGHFRQIDHCHPASVGDFAASRGGGVTVLNYPLNIVWRNHVSLNQSRIRRVESDKSAFNSWALLNERFIGVLNSLIHLDEEPVLRRRRRRRVSLVRQLRRGIAEWLLPIMEWRDSLRDGADTLAAIRETERALLTEPLSDNTKLLKLVNQRFHTTLQDAGYRNTEYAYHPKLLEPVRNQLRWILDTLGNDQFGQRNTQFEVIHLENLRVFDALSLANPYLVGIPSLTALWGFIHAFDRKLKTLLGCEFTFESVAWHVRESSSVSGLKLPSPALERKRSDHLKRPGMIESKHCDLVMDLAIRVHSTEQFLQTRDELVDLIKAALPSRFAGGVIHPPSLYESRDWCSLRTTQSLHEHVSRLPATGRWIVPATTTPKSFENLCELVELNSDLKPAMLGYQLLEEPIERPNSVASLHAYAEPLIGLCDCKSSIDIRLKGEKYFNANFFWKMDTATSSILMRRA

SEQ ID NO: 351 - protein, 162 aa, Enterovibrio coralii
MNGLTGELASALSGEEPFWLADIKANVSASFMQEIFPSQLFSDAKDGSNLGREYAKVRSGDGQIWPSLNAEKIGAAIQLIDDWWADEADKRLRVHEYGGDKKYHIAHRIPSSGIDAYSLLKSVDDKAALLDSLKCSDEIPSDIHYLMAILVKGGLFQKSRSA

SEQ ID NO: 352 - protein, 172 aa, synthetic construct
MPKKKRKVGSGNGLTGELASALSGEEPFWLADIKANVSASFMQEIFPSQLFSDAKDGSNLGREYAKVRSGDGQIWPSLNAEKIGAAIQLIDDWWADEADKRLRVHEYGGDKKYHIAHRIPSSGIDAYSLLKSVDDKAALLDSLKCSDEIPSDIHYLMAILVKGGLFQKSRSA

SEQ ID NO: 353 - protein, 200 aa, Enterovibrio coralii
MKRYYFVITYLPEQASQEILAGRCISTLHDFLVFHHIGGIGVGFPKWTEQSLGNQIMFCSTNQQRLSQLHQSKYFTMMFDQGLFAVTDVEPVPADTAEVRFYRNQGIAKLFTGEKRRRLERAKRRAAERGEMFDPERIGSNQPIGMFHRILMDSQSTQQRFVLHVQKEDVAEASGTDFNGYGLATNRAYRGTVPDIRIPV

SEQ ID NO: 354 - protein, 210 aa, synthetic construct
MPKKKRKVGSGKRYYFVITYLPEQASQEILAGRCISTLHDFLVFHHIGGIGVGFPKWTEQSLGNQIMFCSTNQQRLSQLHQSKYFTMMFDQGLFAVTDVEPVPADTAEVRFYRNQGIAKLFTGEKRRRLERAKRRAAERGEMFDPERIGSNQPIGMFHRILMDSQSTQQRFVLHVQKEDVAEASGTDFNGYGLATNRAYRGTVPDIRIPV

SEQ ID NO: 355 - protein, 640 aa, Vibrio chagasii
MESLKELLQSRPDDLSVDLKRAFRPLTPHINIDGKELDALTVLVNLTDKTADQKDLLDKVKCKQKLRDEKWWARCLKTVEYRQSHNLKFPDIRSEGVIRATPLGQLPDFLLSSSKLEPHNWAYSHDSSDVNKSALLTNEFRWNGVISCLGDLLRDVEHPLWQKFNTLGCYQKTRKAIAKKLAQISQTTINVSLAPNYLTQLSLPDNDSSYISLSPVASQSMQSHCYQALENEYRYTALTRYSRSTNMGVLPMTCGGALKMLKAVPNFSLAPHYQINIGKFWLTSSHIQSLKQYQRHTRYLMPENKRIAYRRTVENEIHEMVKAWLATQDNTMDVNTLVQHLNDDLSRFKSAKCFAYEPNITKLLLGLIKRELTEPTTVSTNICRSEEKNSFFAIPNIRVCGASALSSPITVGLPSLTAFLGFTHAFERNLNESFPTLAIDSFAICIHQLHIEKRGLTKEYVQKANHTISPPATHDDWQCDLVFSLVIKFNRSLNVDENTIVRALPKRFARGSAKIAIADFKYIRSFSTLEKTIQSFPQKAGKWLSMHTEPIKNMSDILSEVKENRKLTPSCVGYHFLEEPTDKPNSLRGYKHAFSECIIGLIEPITFDQNTDINTILWHHKCYQNYLSVQPRSTYHGTTD

SEQ ID NO: 356 - protein, 677 aa, synthetic construct
MPKKKRKVGSGDYKDDDDKDYKDDDDKDYKDDDDKGSGESLKELLQSRPDDLSVDLKRAFRPLTPHINIDGKELDALTVLVNLTDKTADQKDLLDKVKCKQKLRDEKWWARCLKTVEYRQSHNLKFPDIRSEGVIRATPLGQLPDFLLSSSKLEPHNWAYSHDSSDVNKSALLTNEFRWNGVISCLGDLLRDVEHPLWQKFNTLGCYQKTRKAIAKKLAQISQTTINVSLAPNYLTQLSLPDNDSSYISLSPVASQSMQSHCYQALENEYRYTALTRYSRSTNMGVLPMTCGGALKMLKAVPNFSLAPHYQINIGKFWLTSSHIQSLKQYQRHTRYLMPENKRIAYRRTVENEIHEMVKAWLATQDNTMDVNTLVQHLNDDLSRFKSAKCFAYEPNITKLLLGLIKRELTEPTTVSTNICRSEEKNSFFAIPNIRVCGASALSSPITVGLPSLTAFLGFTHAFERNLNESFPTLAIDSFAICIHQLHIEKRGLTKEYVQKANHTISPPATHDDWQCDLVFSLVIKFNRSLNVDENTIVRALPKRFARGSAKIAIADFKYIRSFSTLEKTIQSFPQKAGKWLSMHTEPIKNMSDILSEVKENRKLTPSCVGYHFLEEPTDKPNSLRGYKHAFSECIIGLIEPITFDQNTDINTILWHHKCYQNYLSVQPRSTYHGTTD

SEQ ID NO: 357 - protein, 353 aa, Vibrio chagasii
MELPTNLAYERSIDPSDVCFLVVWPDGRKTPLTYTSRTVLGQMETAALAYDPSGKIKESATAEILAQGNLHQVDFCHAPFGASHIECYFSVSFSSELRKPYKCNSSTVKHTLMQLIKAYEENIGWNELVSRYLVNICNGSWLWKNTKKAYCWDIELTPWPWAGGAVKFQDIRANYLERSDFENHKDWEAIAQMTRNAFSHSNGLAIFEVKATLRLPTNKQIFPSQAFTENESNNTNKSKKKSKGRIFQSTTVDGERSPILGIYKTGAAIATIDDWYPDATEALRVGRFGVHKEDVTCYRHPSTQKDFFSILKQTESYIEALTSSDKPNQETINDLHFLVANIIKGGMFQHKGD

SEQ ID NO: 358 - protein, 363 aa, synthetic construct
MPKKKRKVGSGELPTNLAYERSIDPSDVCFLVVWPDGRKTPLTYTSRTVLGQMETAALAYDPSGKIKESATAEILAQGNLHQVDFCHAPFGASHIECYFSVSFSSELRKPYKCNSSTVKHTLMQLIKAYEENIGWNELVSRYLVNICNGSWLWKNTKKAYCWDIELTPWPWAGGAVKFQDIRANYLERSDFENHKDWEAIAQMTRNAFSHSNGLAIFEVKATLRLPTNKQIFPSQAFTENESNNTNKSKKKSKGRIFQSTTVDGERSPILGIYKTGAAIATIDDWYPDATEALRVGRFGVHKEDVTCYRHPSTQKDFFSILKQTESYIEALTSSDKPNQETINDLHFLVANIIKGGMFQHKGD

SEQ ID NO: 359 - protein, 199 aa, Vibrio chagasii
MKWYYKTVTFLPARCNNESLAAKCLRILHGFNYEYETRNIGVSFPLWSDDTIGNKISFVSTNKIELDLLLKQHYFTQMKDLHYFDISNTKVVPDGCEYVSFKRCQSIDKATPAGQARKAKRLKKRAEERGEEFDLSSFKQHEVVALHHYHSLEEDSKSRGGSFRLNIRIFKEARLDGDALFSSYGLANTENTSQPVPII

SEQ ID NO: 360 - protein, 209 aa, synthetic construct
MPKKKRKVGSGKWYYKTVTFLPARCNNESLAAKCLRILHGFNYEYETRNIGVSFPLWSDDTIGNKISFVSTNKIELDLLLKQHYFTQMKDLHYFDISNTKVVPDGCEYVSFKRCQSIDKATPAGQARKAKRLKKRAEERGEEFDLSSFKQHEVVALHHYHSLEEDSKSRGGSFRLNIRIFKEARLDGDALFSSYGLANTENTSQPVPII

SEQ ID NO: 361 - protein, 640 aa, Vibrio rotiferianus
MRTLAEILKSETDDLNRDLRRAFRPLSPPVDISDFPSEALTILINLTDTVKEQKELLDRSKCKEKLRDEKWWLSCLKTVKYRQSHNPKFPDIRASGIIRAIPMGDIPPFMLSSSKLARCNWAYANDSSQVNKSSFLTSEFIWHNRVHFLGELLTDIEHPLWNILKNLGCYVKTSKEISKKLALIPPHEISTPLARNYLTQISLPDNEDSYISLSPVTSQSIQNNCYETLKEHYRFSSLTRFSRATNMGTLAMSCGGNFRMIHSLPPIEKYKHHHLTDAEQWLTKKSVKALREYTESTHWIISPNKLAKKRKSIIENIRLMLTQWLNTISEREYSNKKELTERFNADLAKTKFASRYAYDPQLTQLIYNSIGSIIQSPPQEVPKPEGTEENYLLLPNLKISGASAMNTPVSIGLPSMTAFYGFVHAFERNLQTVIPNFKIESFAVCIHNLHTENRGLTREWALNTKDEIKAPATRDDWQSDLNVSLILQCSNYSQLVPRDFMYQLPRRLARGKVTVAISAIERLGRSLSLAEAIKTIPVDTGRWLSLNSEAVLNGIQDIIDELKENRMQTVNCIGYHLLELPIEKRCSLRSYKHAFAETILGVMKLFAISENTNPDQYFWKYHYSKQGPILLPRSLSDEAS

SEQ ID NO: 362 - protein, 677 aa, synthetic construct
MPKKKRKVGSGDYKDDDDKDYKDDDDKDYKDDDDKGSGRTLAEILKSETDDLNRDLRRAFRPLSPPVDISDFPSEALTILINLTDTVKEQKELLDRSKCKEKLRDEKWWLSCLKTVKYRQSHNPKFPDIRASGIIRAIPMGDIPPFMLSSSKLARCNWAYANDSSQVNKSSFLTSEFIWHNRVHFLGELLTDIEHPLWNILKNLGCYVKTSKEISKKLALIPPHEISTPLARNYLTQISLPDNEDSYISLSPVTSQSIQNNCYETLKEHYRFSSLTRFSRATNMGTLAMSCGGNFRMIHSLPPIEKYKHHHLTDAEQWLTKKSVKALREYTESTHWIISPNKLAKKRKSIIENIRLMLTQWLNTISEREYSNKKELTERFNADLAKTKFASRYAYDPQLTQLIYNSIGSIIQSPPQEVPKPEGTEENYLLLPNLKISGASAMNTPVSIGLPSMTAFYGFVHAFERNLQTVIPNFKIESFAVCIHNLHTENRGLTREWALNTKDEIKAPATRDDWQSDLNVSLILQCSNYSQLVPRDFMYQLPRRLARGKVTVAISAIERLGRSLSLAEAIKTIPVDTGRWLSLNSEAVLNGIQDIIDELKENRMQTVNCIGYHLLELPIEKRCSLRSYKHAFAETILGVMKLFAISENTNPDQYFWKYHYSKQGPILLPRSLSDEAS

SEQ ID NO: 363 - protein, 348 aa, Vibrio rotiferianus
MKLPNSLSYMRSIDPSDTVFFVNWPNGKRTPLPYSSRTALGRKEGTSSAYKNDDEINEDVTEYSLAHGNPHEIDYCCVPYGAESIECEFSVSFASSLRKPFKCSDPQVKRTLIQLIELYEQKVGWEELATRFLENICNGRWLWRNNERTYSTSISIKPWPWKDEEVIISFNDIRRNYTDINKFRDHEDWEALIKLITDAFSKPNGLCIFEVNATFRLGKNAPIYPSQVFKESIQGEKNRIYQKTEVCGEKSPILGCYKTGAAIATIDDWYHPDAEEPLRISHYGAHKEDVYCYRHPNTGKDLFTLLQRADEYVEQLDAGDVLSDETINDLHFVVANLIKGGLLQRKGS

SEQ ID NO: 364 - protein, 358 aa, synthetic construct
MPKKKRKVGSGKLPNSLSYMRSIDPSDTVFFVNWPNGKRTPLPYSSRTALGRKEGTSSAYKNDDEINEDVTEYSLAHGNPHEIDYCCVPYGAESIECEFSVSFASSLRKPFKCSDPQVKRTLIQLIELYEQKVGWEELATRFLENICNGRWLWRNNERTYSTSISIKPWPWKDEEVIISFNDIRRNYTDINKFRDHEDWEALIKLITDAFSKPNGLCIFEVNATFRLGKNAPIYPSQVFKESIQGEKNRIYQKTEVCGEKSPILGCYKTGAAIATIDDWYHPDAEEPLRISHYGAHKEDVYCYRHPNTGKDLFTLLQRADEYVEQLDAGDVLSDETINDLHFVVANLIKGGLLQRKGS

SEQ ID NO: 365 - protein, 198 aa, Vibrio rotiferianus
MDWYYKTITFLPEYRNNEAIAAKCLKELHSFNYEYKTRSIGISFPLWNQETVGQKITFVSTNKMELDFLLSRRYFTQMTKLGYFSISTAQIVPDDCSYALFRRKQSIDKATPAGQARELKRLERRALERGEIFEPANYSQNTTHAFHNYHSLEENSSGGNGFRLNIQMEQLEDTLSTGKFSSYGLGNTDNSLQVVPLI

SEQ ID NO: 366 - protein, 208 aa, synthetic construct
MPKKKRKVGSGDWYYKTITFLPEYRNNEAIAAKCLKELHSFNYEYKTRSIGISFPLWNQETVGQKITFVSTNKMELDFLLSRRYFTQMTKLGYFSISTAQIVPDDCSYALFRRKQSIDKATPAGQARELKRLERRALERGEIFEPANYSQNTTHAFHNYHSLEENSSGGNGFRLNIQMEQLEDTLSTGKFSSYGLGNTDNSLQVVPLI

SEQ ID NO: 367 - protein, 640 aa, Vibrio parahaemolyticus
MATLAEILDNKTDDLNKDLRRAFRPLSAPVDISDTPIEALTILVNLTDRVIEQKNLLDRQKCKDKLRDEKWWANCFRTVKYRQSHNPKFPDIRANGVIRAAPVGHLPACMLSSSKLPQNSWAYANDSSQMNKSCFLTSEFIWNGDVHCLGQLLTELEHPLWNVLRKLGCYVKTAKYISKELALIPPLEINTSLVRNYLAQISLPNNEDSYISLSPVVSQSMQEDCYQVLSEHYRFSAITRFSRATNMGTLAMSCGGKFKMIRSLPPIEKYQHHHLDSVNWLTKRSVRAIRDYTESSVWVISPNKLALRKKSIIGDIKMMLSQWLRTTPTHEEKLDIRKLTERFNVDLAKTKFANRYAYDPLLTQLIYNCIGSIIHSPPQYAPKCEGNDDKYLLLPNLRISGASAMNTSVSIGIPSMMAFYGFVHAFQRNVQTANPNFKIESFAVCIHNIHVENRGLTREWVPNTKGQITAPATRDDWQCDVAVSLILRCSHYSQLIPRDFIRLLPGRIARGKVTVSISDIKHLGRCLSLADAIKAIPVETGRWLSLNNEVTLNSIQDVIDELKNNKLQTVNCIGYHRLETPCEKRGSLHGYKHAFVETILGIIKFLTISENTNPSQYFWQYHYSKQGPILLPRSVSDETS

SEQ ID NO: 368 - protein, 677 aa, synthetic construct
MPKKKRKVGSGDYKDDDDKDYKDDDDKDYKDDDDKGSGATLAEILDNKTDDLNKDLRRAFRPLSAPVDISDTPIEALTILVNLTDRVIEQKNLLDRQKCKDKLRDEKWWANCFRTVKYRQSHNPKFPDIRANGVIRAAPVGHLPACMLSSSKLPQNSWAYANDSSQMNKSCFLTSEFIWNGDVHCLGQLLTELEHPLWNVLRKLGCYVKTAKYISKELALIPPLEINTSLVRNYLAQISLPNNEDSYISLSPVVSQSMQEDCYQVLSEHYRFSAITRFSRATNMGTLAMSCGGKFKMIRSLPPIEKYQHHHLDSVNWLTKRSVRAIRDYTESSVWVISPNKLALRKKSIIGDIKMMLSQWLRTTPTHEEKLDIRKLTERFNVDLAKTKFANRYAYDPLLTQLIYNCIGSIIHSPPQYAPKCEGNDDKYLLLPNLRISGASAMNTSVSIGIPSMMAFYGFVHAFQRNVQTANPNFKIESFAVCIHNIHVENRGLTREWVPNTKGQITAPATRDDWQCDVAVSLILRCSHYSQLIPRDFIRLLPGRIARGKVTVSISDIKHLGRCLSLADAIKAIPVETGRWLSLNNEVTLNSIQDVIDELKNNKLQTVNCIGYHRLETPCEKRGSLHGYKHAFVETILGIIKFLTISENTNPSQYFWQYHYSKQGPILLPRSVSDETS

SEQ ID NO: 369 - protein, 347 aa, Vibrio parahaemolyticus
MKLPNGLSYMKSIEASDVIFLVNWPDGRKTPLPYTSRVALGMKEGSKSAYKYDGQIDADVTAYSLAQGNPHEIDFCCVPYGAESIECEFSVSFASSLRKPFKCSDPEVKRTLVQLIKLYEEKVGWEELANRFLENICNGRWLWRNNECTYSTSIGIKPWPWEDEKAISPFHDIRKNYAGTNHFRDHKDWDNLIKLITDAFSQPNGLCIFEVSATFRLGTNAPIYPSQVFKDSVKGEKNRIYQSTDVDGESSPILGCYKTGAAIATIDDWYPDADKPIRISHYGAHREDVYCYRHPNTGKDLFTLLEKADQYLEQLQATDVLPDEMINDLHFIVANLIKGGLLQQKGT

SEQ ID NO: 370 - protein, 357 aa, synthetic construct
MPKKKRKVGSGKLPNGLSYMKSIEASDVIFLVNWPDGRKTPLPYTSRVALGMKEGSKSAYKYDGQIDADVTAYSLAQGNPHEIDFCCVPYGAESIECEFSVSFASSLRKPFKCSDPEVKRTLVQLIKLYEEKVGWEELANRFLENICNGRWLWRNNECTYSTSIGIKPWPWEDEKAISPFHDIRKNYAGTNHFRDHKDWDNLIKLITDAFSQPNGLCIFEVSATFRLGTNAPIYPSQVFKDSVKGEKNRIYQSTDVDGESSPILGCYKTGAAIATIDDWYPDADKPIRISHYGAHREDVYCYRHPNTGKDLFTLLEKADQYLEQLQATDVLPDEMINDLHFIVANLIKGGLLQQKGT

SEQ ID NO: 371 - protein, 198 aa, Vibrio parahaemolyticus
MDWHYRTITFLPEYRNNEAIAAKCIKELHRFNYKYETRSIGVSFPLWGQETVGRKITFVSTNKMELDFLISRRYFVQMTKLGYFSISTTQTVPDDCSYVLFKRAHSIDKGTFAGRARELKRLERRALERGEIFDPIAYSKTTSHAFQSYHSLEEDSSSGNKFRLNIQMKERSGTVGTGKFSSYGLGNTDNSLQVVPLI

SEQ ID NO: 372 - protein, 208 aa, synthetic construct
MPKKKRKVGSGDWHYRTITFLPEYRNNEAIAAKCIKELHRFNYKYETRSIGVSFPLWGQETVGRKITFVSTNKMELDFLISRRYFVQMTKLGYFSISTTQTVPDDCSYVLFKRAHSIDKGTFAGRARELKRLERRALERGEIFDPIAYSKTTSHAFQSYHSLEEDSSSGNKFRLNIQMKERSGTVGTGKFSSYGLGNTDNSLQVVPLI

SEQ ID NO: 373 - protein, 640 aa, Vibrio parahaemolyticus
MATLAEILDNKTDDLNKDLRRAFRPLSAPVDISDTPIEALTILVNLTDRVIEQKDLLDRKKCKDKLRDEKWWADCFRTVKYRQSHNPKFPDIRANGVIRAAPVGHLPPFMLSSSKLPQNSWAYANDSGQVNKSCFLTSEFIWNGDVLCLGQLLTELEHPLWNVLRKLGCYVKTAKYISKELALIPPLEINTSLVRNYLAQISLPNDEDSYISLSPVASQSMQEDCYQVLSEHCRFSAITRFSRATNMGTLAMSCGGKFKMIRSLPPIEKYQHHHLDSVNWLTKRSVRAIRDYTESSVWVISPNKLALRKKSIIEDIKIMLSQWLRTTPTHEEKLDIRKLTERFNVDLAKTEFANRYAYDPLLTQLIYNCIGSIIHSPPQDAPKCEGNDDKYLLLPNLRISGASAMNTSVSIGIPSMMAFYGFVHAFQRNVQTANPNFKIESFAVCIHNIHVENRGLTREWVPNTKGQITAPATRDDWQCDVAVSLILRCSHYSQLIPRDFIRLLPGRIARGKVTVSISDIKHLGRCLSLADAIKAIPVETGRWLSLNNEVTLNSIQDVIDELKNNRLQTVSCIGYQLLEPPCEKRGSLHGYKHAFVETILGIIKLLAISKNTNPDQYFWQYHYSKQGPILLLKSISDETS

SEQ ID NO: 374 - protein, 677 aa, synthetic construct
MPKKKRKVGSGDYKDDDDKDYKDDDDKDYKDDDDKGSGATLAEILDNKTDDLNKDLRRAFRPLSAPVDISDTPIEALTILVNLTDRVIEQKDLLDRKKCKDKLRDEKWWADCFRTVKYRQSHNPKFPDIRANGVIRAAPVGHLPPFMLSSSKLPQNSWAYANDSGQVNKSCFLTSEFIWNGDVLCLGQLLTELEHPLWNVLRKLGCYVKTAKYISKELALIPPLEINTSLVRNYLAQISLPNDEDSYISLSPVASQSMQEDCYQVLSEHCRFSAITRFSRATNMGTLAMSCGGKFKMIRSLPPIEKYQHHHLDSVNWLTKRSVRAIRDYTESSVWVISPNKLALRKKSIIEDIKIMLSQWLRTTPTHEEKLDIRKLTERFNVDLAKTEFANRYAYDPLLTQLIYNCIGSIIHSPPQDAPKCEGNDDKYLLLPNLRISGASAMNTSVSIGIPSMMAFYGFVHAFQRNVQTANPNFKIESFAVCIHNIHVENRGLTREWVPNTKGQITAPATRDDWQCDVAVSLILRCSHYSQLIPRDFIRLLPGRIARGKVTVSISDIKHLGRCLSLADAIKAIPVETGRWLSLNNEVTLNSIQDVIDELKNNRLQTVSCIGYQLLEPPCEKRGSLHGYKHAFVETILGIIKLLAISKNTNPDQYFWQYHYSKQGPILLLKSISDETS

SEQ ID NO: 375 - protein, 347 aa, Vibrio parahaemolyticus
MKLPNNLSYIKSIEPSDVIFLVNWPDGRKTPLPYTSRVALGMKEGSKSAYKDDGQIDMDATAHSLAHGNAHEIDFCCVPYGAESIECEFSVSFASSLRKPFKCSDPEVKRTLVQLIKLYEEKVGWEELANRFLENICNGRWLWRNNECTYSTSIGIKPWPWEDEKAISPFHDIRKNYAGTNHFRDHKDWDKLIKLITDAFSQPNGLCIFEVSATFRLGTNAPIYPSQVFKDSVKGEKNRIYQSTNVDGESSPILGCYKTGAAIATIDDWYPDADKPIRISHYGAHKEDVYCYRHPNTGKDLFTLLEKADQYLEQLQATEVLPDEMINDLHFIVANLIKGGLLQRKGT

SEQ ID NO: 376 - protein, 357 aa, synthetic construct
MPKKKRKVGSGKLPNNLSYIKSIEPSDVIFLVNWPDGRKTPLPYTSRVALGMKEGSKSAYKDDGQIDMDATAHSLAHGNAHEIDFCCVPYGAESIECEFSVSFASSLRKPFKCSDPEVKRTLVQLIKLYEEKVGWEELANRFLENICNGRWLWRNNECTYSTSIGIKPWPWEDEKAISPFHDIRKNYAGTNHFRDHKDWDKLIKLITDAFSQPNGLCIFEVSATFRLGTNAPIYPSQVFKDSVKGEKNRIYQSTNVDGESSPILGCYKTGAAIATIDDWYPDADKPIRISHYGAHKEDVYCYRHPNTGKDLFTLLEKADQYLEQLQATEVLPDEMINDLHFIVANLIKGGLLQRKGT

SEQ ID NO: 377 - protein, 198 aa, Vibrio parahaemolyticus
MDWYYRTITFLPEYRNNEAIAAKCIKELHRFNYKYETRSIGVSFPLWGQETVGRKITFVSTNKMELDFLISRRYFVQMTKLGYFSISTTQTVPDDCSYVLFKRAHSIDKGTSAGRARELKRLERRALERGEIFDPMAYSKTTSHAFQSYHSLEEDSSSGNKFRLNIQMKERSGTVDTGTFSSYGLGNTDNSLQVVPLI

SEQ ID NO: 378 - protein, 208 aa, synthetic construct
MPKKKRKVGSGDWYYRTITFLPEYRNNEAIAAKCIKELHRFNYKYETRSIGVSFPLWGQETVGRKITFVSTNKMELDFLISRRYFVQMTKLGYFSISTTQTVPDDCSYVLFKRAHSIDKGTSAGRARELKRLERRALERGEIFDPMAYSKTTSHAFQSYHSLEEDSSSGNKFRLNIQMKERSGTVDTGTFSSYGLGNTDNSLQVVPLI

SEQ ID NO: 379 - protein, 690 aa, Vibrio fischeri
MEFTDILIIQDVKERNRALKVAFAHYSSAICIDEHEVEAITCLLNLCTPKTEDYLDKTSASLFLNNHDNIQKCLDELKWFHSHNVKYPDCRVKGQSIISLPIDSVSNTINSNVVPYRLGWSHDSGKVNYTHFLLSCFKWRGVQTTLSQLFITDTLFWLDIIKKIQCNWTKKQTEQFIHSIQKEMPAKTLPENISPYSKQILFPYKNDYLTLTPVTSNSVQTWLEHQSRKPDDIRWIKRESKHSASVGALSSSIGGYHSLLFSPPSTSQSPHSYHDNMASKTGCREAFCTSAITEKSTTDALQRLISSEVRMNVKHRKKIRKSGVHFIRQKIALWLTPLIRWRDHIDNNQIQITNDHPSLVNLFLSSPIASFPDLLAPLHNHLNQTLGKNKYTKRFAYHPDLMPIFKSQLSWILNKLAQDENINQQPVLPRTQFIHLKNLRLYNGNALSSPYVCGLPSLTGFWGFMHDFERRLKTKIEENIHFEAFSLFVHQYELQSSPPLCEASDIYKKRELSPAKRLLTQPSYSCDMRFDLIIKVHTEVNLSDISQRMLSAMPARCVGGTLHQSSLHESLEWLTSYASSEHLYEELACLPNSGRWIYPPSETFNTPDEFLSILGNSTHLAICNGYSFLEDPTNRENVSLNQHVFCEPLIGLAEQVIPIDMRLNRQKYYFSNAFWSINSDFNSILIQKHE

SEQ ID NO: 380 - protein, 727 aa, synthetic construct
MPKKKRKVGSGDYKDDDDKDYKDDDDKDYKDDDDKGSGEFTDILIIQDVKERNRALKVAFAHYSSAICIDEHEVEAITCLLNLCTPKTEDYLDKTSASLFLNNHDNIQKCLDELKWFHSHNVKYPDCRVKGQSIISLPIDSVSNTINSNVVPYRLGWSHDSGKVNYTHFLLSCFKWRGVQTTLSQLFITDTLFWLDIIKKIQCNWTKKQTEQFIHSIQKEMPAKTLPENISPYSKQILFPYKNDYLTLTPVTSNSVQTWLEHQSRKPDDIRWIKRESKHSASVGALSSSIGGYHSLLFSPPSTSQSPHSYHDNMASKTGCREAFCTSAITEKSTTDALQRLISSEVRMNVKHRKKIRKSGVHFIRQKIALWLTPLIRWRDHIDNNQIQITNDHPSLVNLFLSSPIASFPDLLAPLHNHLNQTLGKNKYTKRFAYHPDLMPIFKSQLSWILNKLAQDENINQQPVLPRTQFIHLKNLRLYNGNALSSPYVCGLPSLTGFWGFMHDFERRLKTKIEENIHFEAFSLFVHQYELQSSPPLCEASDIYKKRELSPAKRLLTQPSYSCDMRFDLIIKVHTEVNLSDISQRMLSAMPARCVGGTLHQSSLHESLEWLTSYASSEHLYEELACLPNSGRWIYPPSETFNTPDEFLSILGNSTHLAICNGYSFLEDPTNRENVSLNQHVFCEPLIGLAEQVIPIDMRLNRQKYYFSNAFWSINSDFNSILIQKHE

SEQ ID NO: 381 - protein, 344 aa, Vibrio fischeri
MKLCNNLNYTRSLSPGKAVFYYESKDGQMNPIKCEQTHLRAPKAGFSEAFNSDYSTKNTAPQDLSFSNPQFIEECYVPVGIDEIKIRFSLRIEANSLQPDKCSDVQIREILQAFATKYKENGGYQELGERYAKNLLSGTWLWRNEHNLGTSISIKTTSNQEFNIDNAFKLSRKTSAKDKKTISKLGSEIASALSDPDHYYFADITATINVAFCQEIYPSQEFLDTKEKGKPSKVYAKTSLLTDEKTVALHAQKIGAAIQLIDDWWADDADIPLRVNEFGADHHNVIARRHPSHRNDFYTLIQNADNYCAQLDENSDITDDMHYVMAVLVKGGLFQKSASSKKGK

SEQ ID NO: 382 - protein, 354 aa, synthetic construct
MPKKKRKVGSGKLCNNLNYTRSLSPGKAVFYYESKDGQMNPIKCEQTHLRAPKAGFSEAFNSDYSTKNTAPQDLSFSNPQFIEECYVPVGIDEIKIRFSLRIEANSLQPDKCSDVQIREILQAFATKYKENGGYQELGERYAKNLLSGTWLWRNEHNLGTSISIKTTSNQEFNIDNAFKLSRKTSAKDKKTISKLGSEIASALSDPDHYYFADITATINVAFCQEIYPSQEFLDTKEKGKPSKVYAKTSLLTDEKTVALHAQKIGAAIQLIDDWWADDADIPLRVNEFGADHHNVIARRHPSHRNDFYTLIQNADNYCAQLDENSDITDDMHYVMAVLVKGGLFQKSASSKKGK

SEQ ID NO: 383 - protein, 213 aa, Vibrio fischeri
MLTHYFSITYVPDDCDNELLAGRCIAEFHKFISSLRLIENNSFAIGFPNWSEQSVGNEFAIFSDNSELLSAIKYQPYFNLMRNEELFSITDIKPVPNNLPQIRFIRNQSIGKIFIGSKKRRIQRSITRNNKEHTPISNEDREFDTFHKVSCSSKSKQQQYILHIQKDITPRTIDSKGSYNSYGLATNSKHLGTVPDLSKIPFYCEEKLSNKDQ

SEQ ID NO: 384 - protein, 223 aa, synthetic construct
MPKKKRKVGSGLTHYFSITYVPDDCDNELLAGRCIAEFHKFISSLRLIENNSFAIGFPNWSEQSVGNEFAIFSDNSELLSAIKYQPYFNLMRNEELFSITDIKPVPNNLPQIRFIRNQSIGKIFIGSKKRRIQRSITRNNKEHTPISNEDREFDTFHKVSCSSKSKQQQYILHIQKDITPRTIDSKGSYNSYGLATNSKHLGTVPDLSKIPFYCEEKLSNKDQ

SEQ ID NO: 385 - protein, 642 aa, Vibrio parahaemolyticus
MTLDELLAATDLEELVSSTKRAFRPLSPLIDITQNPLNALTILINLTEKGISNKNLLDRTRCKEKLRDDKWWAAVLKPAQYRHSHNVKFPDIRSTGTIRTIAPDNLPAYFITSSKLPNVGWTYSKDSSDINRCLFFTSEFLWAGQACCLAKTLTDSEHPLWSTLKKMGCYEKHKNLAVKLLSQIPDELIDVDLSGNYLSQVSFPDGHDSYLSFSPVASQAMQSCVYQSLEQHYRQTALMGFDRATNMGLLAASCGGRFRLIETKTYIKDKRHHYISEQPNWLTKEAIQSIEQFLSSEQWLVTHNDKPRNMAIVKSSIRTMVNRWLSTRTITEDLSPAALTEQLNADMASIRIIKRYAYQPKLTRLFIQLIESAVEDNDYKEDREATTNSQYLLIPELRISGGSAKSSSASVGLFSMMSLYGFIHAFERNMRHVLTNFTINSFAICIHDYHLEKRGLTKEPIKKAKVSRDEKEKIAPPAIYDDYQFDSCISLIIKTSESKTIPAEKIVALLPKRFARGSIRLFIDGIKNIAPFPEPLPAIQAINNPHGSWLSFEPDLSLTSTDSLVDITINRSNLLLTVMGYQYLEPPTTKPGSLRDYPHALVENILGFVKPRTVTQSTNLDDLFWRYQVTHFGVCLLPRSIK

SEQ ID NO: 386 - protein, 679 aa, synthetic construct
MPKKKRKVGSGDYKDDDDKDYKDDDDKDYKDDDDKGSGTLDELLAATDLEELVSSTKRAFRPLSPLIDITQNPLNALTILINLTEKGISNKNLLDRTRCKEKLRDDKWWAAVLKPAQYRHSHNVKFPDIRSTGTIRTIAPDNLPAYFITSSKLPNVGWTYSKDSSDINRCLFFTSEFLWAGQACCLAKTLTDSEHPLWSTLKKMGCYEKHKNLAVKLLSQIPDELIDVDLSGNYLSQVSFPDGHDSYLSFSPVASQAMQSCVYQSLEQHYRQTALMGFDRATNMGLLAASCGGRFRLIETKTYIKDKRHHYISEQPNWLTKEAIQSIEQFLSSEQWLVTHNDKPRNMAIVKSSIRTMVNRWLSTRTITEDLSPAALTEQLNADMASIRIIKRYAYQPKLTRLFIQLIESAVEDNDYKEDREATTNSQYLLIPELRISGGSAKSSSASVGLFSMMSLYGFIHAFERNMRHVLTNFTINSFAICIHDYHLEKRGLTKEPIKKAKVSRDEKEKIAPPAIYDDYQFDSCISLIIKTSESKTIPAEKIVALLPKRFARGSIRLFIDGIKNIAPFPEPLPAIQAINNPHGSWLSFEPDLSLTSTDSLVDITINRSNLLLTVMGYQYLEPPTTKPGSLRDYPHALVENILGFVKPRTVTQSTNLDDLFWRYQVTHFGVCLLPRSIK

SEQ ID NO: 387 - protein, 345 aa, Vibrio parahaemolyticus
MKLPIHLAYERSISPSDVAFLVVWPDGNKKPLPCYSRTILGLNEGSHVGYDDSGTVRNNLKMNTLVDGNIHELDYCSVPYGAKSIECCFSVSFSSELLKPYKCSDADVKKTLREFINLYNQRVELDELIIKYLTNIALGTWLWHNTKRSYCVSIEVRPWPWEGEPIIIDDIRKYLKGESDTNDLLNWKKLIKQVKEAFTDPMGLCILEVKANLIKPSMAQLYPSQMFKEAAKKENNRLYQSTIIDGIKSPIMGCYKTGAAIAKIDTWYPDAEEPIRVGHYGVDRENSTAYRHPSTGKDFFSILKRTDEFVDRLKDSEELNQDNLNDMHFLMANLIKGGLFQEKGE

SEQ ID NO: 388 - protein, 355 aa, synthetic construct
MPKKKRKVGSGKLPIHLAYERSISPSDVAFLVVWPDGNKKPLPCYSRTILGLNEGSHVGYDDSGTVRNNLKMNTLVDGNIHELDYCSVPYGAKSIECCFSVSFSSELLKPYKCSDADVKKTLREFINLYNQRVELDELIIKYLTNIALGTWLWHNTKRSYCVSIEVRPWPWEGEPIIIDDIRKYLKGESDTNDLLNWKKLIKQVKEAFTDPMGLCILEVKANLIKPSMAQLYPSQMFKEAAKKENNRLYQSTIIDGIKSPIMGCYKTGAAIAKIDTWYPDAEEPIRVGHYGVDRENSTAYRHPSTGKDFFSILKRTDEFVDRLKDSEELNQDNLNDMHFLMANLIKGGLFQEKGE

SEQ ID NO: 389 - protein, 197 aa, Vibrio parahaemolyticus
MMLYYRTVTFLPKIKNNEALIGHCLKVLHGVCTKYTINTIGVSFPEWGKESIGDKISFISPKPLELDFLLQQNYFAEMTALGYFSISESTTVPEECNLAVFRRNQKIDQATPNGQRIRAERLAKRAMNRGDSPIRFIPKDHVFEHYHSIPITSTQSGKSFRLNLQYQQLGTVTDGEWAFSSYGLANQKLKSSPVPVI

SEQ ID NO: 390 - protein, 207 aa, synthetic construct
MPKKKRKVGSGMLYYRTVTFLPKIKNNEALIGHCLKVLHGVCTKYTINTIGVSFPEWGKESIGDKISFISPKPLELDFLLQQNYFAEMTALGYFSISESTTVPEECNLAVFRRNQKIDQATPNGQRIRAERLAKRAMNRGDSPIRFIPKDHVFEHYHSIPITSTQSGKSFRLNLQYQQLGTVTDGEWAFSSYGLANQKLKSSPVPVI

SEQ ID NO: 391 - protein, 695 aa, Vibrio cholerae
MTKLSDLLVIEDEAIKQTALKKMFMPYTEDVCVDGYEQETLTILLNLSSSHQADRCSDWLDVARAQRYLKDRENLDASLAEIQWFHTHNLKFPDCRVKDQRIIARPLSTAEEFISSAVLDQRLGWAHNSAVYRHTLWLLNPFKWQSQPVCILSLIQQKNPVWLDLLTEFGLDVKSLARLQRAIEEQLPENSFPNSVSAYSKQLRFPWGDDYVSITPVVSHALQCELEIRARSPENKFSFVSSSLPNSASIGNLCGSLGGYMRVLNYPLGVKQAKGGTLTGNRQKSGHYFDDYQVTNAKICQVLNRLIGSEPSKTQRQRERARQVRGKILRKQIALWMLPLIELRDIAESEPNQQQLEHDDTLAQAFLSLPELELGSLAGEFNRRLHLTFQNNIYSAKFAYHPKLMQVAKAQVTWVLEQLSKPINNQDKVTGEQYIYLSSMRVQDAVAMSNPCLCGVPSLTAIWGVMHDYQRKFNQLVNNGSPVEFSSFAFYVRNENIQSTAKLTEPNSVAKARTVSNAKRPTIRSERLSDLEIDLVIRVHSESRISDFRSALKTALPVAFAGGALYQPHLSTQIEWLRTFTGRSELFHVLKGLPAYGRWLYPSEKQPTNFDELERLLTQDDDNLPVSLGYHLLEHPTKRDNAITGCHAYAENAIGLAKRINPIEVRFSGRDHFLNHAFWSIECSSETILIKNYRD

SEQ ID NO: 392 - protein, 732 aa, synthetic construct
MPKKKRKVGSGDYKDDDDKDYKDDDDKDYKDDDDKGSGTKLSDLLVIEDEAIKQTALKKMFMPYTEDVCVDGYEQETLTILLNLSSSHQADRCSDWLDVARAQRYLKDRENLDASLAEIQWFHTHNLKFPDCRVKDQRIIARPLSTAEEFISSAVLDQRLGWAHNSAVYRHTLWLLNPFKWQSQPVCILSLIQQKNPVWLDLLTEFGLDVKSLARLQRAIEEQLPENSFPNSVSAYSKQLRFPWGDDYVSITPVVSHALQCELEIRARSPENKFSFVSSSLPNSASIGNLCGSLGGYMRVLNYPLGVKQAKGGTLTGNRQKSGHYFDDYQVTNAKICQVLNRLIGSEPSKTQRQRERARQVRGKILRKQIALWMLPLIELRDIAESEPNQQQLEHDDTLAQAFLSLPELELGSLAGEFNRRLHLTFQNNIYSAKFAYHPKLMQVAKAQVTWVLEQLSKPINNQDKVTGEQYIYLSSMRVQDAVAMSNPCLCGVPSLTAIWGVMHDYQRKFNQLVNNGSPVEFSSFAFYVRNENIQSTAKLTEPNSVAKARTVSNAKRPTIRSERLSDLEIDLVIRVHSESRISDFRSALKTALPVAFAGGALYQPHLSTQIEWLRTFTGRSELFHVLKGLPAYGRWLYPSEKQPTNFDELERLLTQDDDNLPVSLGYHLLEHPTKRDNAITGCHAYAENAIGLAKRINPIEVRFSGRDHFLNHAFWSIECSSETILIKNYRD

SEQ ID NO: 393 - protein, 343 aa, Vibrio cholerae
MELCTQLNYVRSLSAGKAYFYYLSESGEMCPLNVDKTRLRAPKGSYSEAYKGNKFVDKNVAPQDLAYSNPQFIEECYVKPGVDEIYCAFSLRIRANSLTPDICSDDEVRSKLSMFSKIYKELNGYKELANRYAKNILLGTWLWRNRECRNITIEVTTSELDTFVVEHAQKLSWYGHWDGDSTECLERLTAYLERALSDPTEYFYMDVKAKMRVGWGDEVYPSQEFLDSREDGIPTKQLATVELLRGKETVAFHGQKVGAALQSIDDWWHEEADKPLRVNEYGADREYVIARRHVTHGNDFYQLVRNTENWIEAMTASQTIPNDVHFIMSVLIKGGLFNCAKAN

SEQ ID NO: 394 - protein, 353 aa, synthetic construct
MPKKKRKVGSGELCTQLNYVRSLSAGKAYFYYLSESGEMCPLNVDKTRLRAPKGSYSEAYKGNKFVDKNVAPQDLAYSNPQFIEECYVKPGVDEIYCAFSLRIRANSLTPDICSDDEVRSKLSMFSKIYKELNGYKELANRYAKNILLGTWLWRNRECRNITIEVTTSELDTFVVEHAQKLSWYGHWDGDSTECLERLTAYLERALSDPTEYFYMDVKAKMRVGWGDEVYPSQEFLDSREDGIPTKQLATVELLRGKETVAFHGQKVGAALQSIDDWWHEEADKPLRVNEYGADREYVIARRHVTHGNDFYQLVRNTENWIEAMTASQTIPNDVHFIMSVLIKGGLFNCAKAN

SEQ ID NO: 395 - protein, 201 aa, Vibrio cholerae
MSQRYYFLIRYTNANADYGLLAGRCISQTHLFMVNNHQAMNRVGVSFPDWNESSVGQTIAFVSEDKEMMIGLSFQPYFSLMVKEGLFELSSICEVPDNLGEVRFVRNQTINKSFLGSKKRRIKRSMVRAELSGAEQRLPVTNEDRVIDSFHRIPISSGSSRQDFILFIQKELADERAESGFNSYALATNQERRGTVPDLRF

SEQ ID NO: 396 - protein, 211 aa, synthetic construct
MPKKKRKVGSGSQRYYFLIRYTNANADYGLLAGRCISQTHLFMVNNHQAMNRVGVSFPDWNESSVGQTIAFVSEDKEMMIGLSFQPYFSLMVKEGLFELSSICEVPDNLGEVRFVRNQTINKSFLGSKKRRIKRSMVRAELSGAEQRLPVTNEDRVIDSFHRIPISSGSSRQDFILFIQKELADERAESGFNSYALATNQERRGTVPDLRF

SEQ ID NO: 397 - protein, 689 aa, Agarivorans gilvus
MTLADIITTQNIAERNRALKRAFAPDSNGVEVVGKEQEALVVLLNLSLRKEEVDDLCDQTLATTTLRNQKHLQLCCSEIQWLHSHNLKFPNARVSHQRLLTSPQVPVSGTLSSANFPVRYGWSHDSARIRKASLFCAEFKWNGLWTCLAKELDERDHIWQKVFFELGFSRRDFQALTAMVGELLGEETFPQEVSPFSSQIRVPFKNSYCSVTPVVSHSLQSAIQNLDYILKKGKFKRLQHEHSASIGNLCAAHGGRVSSLFYPPHIIKYQHVTLSSSLEKRSKSDSVFNRKAINNKIFHNALRALINPSVEITLKKRRQRRLSALRYVRKELAAWLAPVMEWRDSLEETEGTLNELEQDSLVYRLLTFEPCDFPVLLNQLNICLHEELQTSFYGAEFAFHPRLIHPLKSQLLWLLNYLGKDDDESDVESDVQYIYFSNLRVFDADAMANPYLCGIPSLTAVWGMCHRFQLQLNKLLPESVSVDGFTWFVHQYSLSAGRKLPEPSRYIRNELKRPGFIAGQHCDLTIDLILKISAREDFRLSDDDIPLIQASLPAKLAGGSVHPPSLYERREWCSLYSVQHELFDRLARLPTGGRWVFPTHQEVHSLEELMDIITSDYSIKPAMLGYLLLEEPTLREGALTSMHAYAEPLLGLVQTLSAIDVRIMKPKVFWAAAFWQLKVSERAMLMKSL

SEQ ID NO: 398 - protein, 726 aa, synthetic construct
MPKKKRKVGSGDYKDDDDKDYKDDDDKDYKDDDDKGSGTLADIITTQNIAERNRALKRAFAPDSNGVEVVGKEQEALVVLLNLSLRKEEVDDLCDQTLATTTLRNQKHLQLCCSEIQWLHSHNLKFPNARVSHQRLLTSPQVPVSGTLSSANFPVRYGWSHDSARIRKASLFCAEFKWNGLWTCLAKELDERDHIWQKVFFELGFSRRDFQALTAMVGELLGEETFPQEVSPFSSQIRVPFKNSYCSVTPVVSHSLQSAIQNLDYILKKGKFKRLQHEHSASIGNLCAAHGGRVSSLFYPPHIIKYQHVTLSSSLEKRSKSDSVFNRKAINNKIFHNALRALINPSVEITLKKRRQRRLSALRYVRKELAAWLAPVMEWRDSLEETEGTLNELEQDSLVYRLLTFEPCDFPVLLNQLNICLHEELQTSFYGAEFAFHPRLIHPLKSQLLWLLNYLGKDDDESDVESDVQYIYFSNLRVFDADAMANPYLCGIPSLTAVWGMCHRFQLQLNKLLPESVSVDGFTWFVHQYSLSAGRKLPEPSRYIRNELKRPGFIAGQHCDLTIDLILKISAREDFRLSDDDIPLIQASLPAKLAGGSVHPPSLYERREWCSLYSVQHELFDRLARLPTGGRWVFPTHQEVHSLEELMDIITSDYSIKPAMLGYLLLEEPTLREGALTSMHAYAEPLLGLVQTLSAIDVRIMKPKVFWAAAFWQLKVSERAMLMKSL

SEQ ID NO: 399 - protein, 344 aa, Agarivorans gilvus
MQLCKQLKYERSIQPGKAVFFYKTEDSEFVPLEADIKRIRGQKTSFSEAYASIAKPKNVAVQDLAYSNPIRMETVTVPPLVEAIYCRFNLRIFANSLEPSVCDDLDTHNILKQLANGYRQKEGYKELAKRYAKNLLLGQWLFRNQQTYPVSIELLTSNNSIFSVNDVHQFDWNSRSNSYINQVEKLAAELAGAFSEPRRYWSAEVTAKISAQMGEEIFPSQQLTEKVEKGEISKLFCKLAMPDGREAVILNMEKVGAGIQMIDDWYTDEADYRLRVHEYGADPKHVIAQRRPETHSDFYSLVSQAEAHLEVLKQAVSSSDIPAEIHYVMSVLIKGGMFQRGKEG

SEQ ID NO: 400 - protein, 354 aa, synthetic construct
MPKKKRKVGSGQLCKQLKYERSIQPGKAVFFYKTEDSEFVPLEADIKRIRGQKTSFSEAYASIAKPKNVAVQDLAYSNPIRMETVTVPPLVEAIYCRFNLRIFANSLEPSVCDDLDTHNILKQLANGYRQKEGYKELAKRYAKNLLLGQWLFRNQQTYPVSIELLTSNNSIFSVNDVHQFDWNSRSNSYINQVEKLAAELAGAFSEPRRYWSAEVTAKISAQMGEEIFPSQQLTEKVEKGEISKLFCKLAMPDGREAVILNMEKVGAGIQMIDDWYTDEADYRLRVHEYGADPKHVIAQRRPETHSDFYSLVSQAEAHLEVLKQAVSSSDIPAEIHYVMSVLIKGGMFQRGKEG

SEQ ID NO: 401 - protein, 195 aa, Agarivorans gilvus
VIERYYFIVRYLPKRADCSLLAGRCIKELHHIFSQTEESIAVSFPEWTVGSLGPSIGFVSSSVKYLEALRNRSYFIDMQEIGAFELTKVLTVPNEVGEVRFIRNQRVAKLFSGEFRRRYARGKKRPKLGGKALIRNTCQRMLRSPHSIRLLYRVVQVSSISFFIYKKSLPKLLKPQGFVVTALLRHARKGELFQT

SEQ ID NO: 402 - protein, 205 aa, synthetic construct
MPKKKRKVGSGIERYYFIVRYLPKRADCSLLAGRCIKELHHIFSQTEESIAVSFPEWTVGSLGPSIGFVSSSVKYLEALRNRSYFIDMQEIGAFELTKVLTVPNEVGEVRFIRNQRVAKLFSGEFRRRYARGKKRPKLGGKALIRNTCQRMLRSPHSIRLLYRVVQVSSISFFIYKKSLPKLLKPQGFVVTALLRHARKGELFQT

SEQ ID NO: 403 - protein, 695 aa, Vibrio cholerae; identical to SEQ ID NO: 265

SEQ ID NO: 404 - protein, 732 aa, synthetic construct; identical to SEQ ID NO: 266

SEQ ID NO: 405 - protein, 343 aa, Vibrio cholerae; identical to SEQ ID NO: 267

SEQ ID NO: 406 - protein, 353 aa, synthetic construct; identical to SEQ ID NO: 268

SEQ ID NO: 407 - protein, 209 aa, Vibrio cholerae; identical to SEQ ID NO: 269

SEQ ID NO: 408 - protein, 219 aa, synthetic construct; identical to SEQ ID NO: 270

SEQ ID NO: 409 - protein, 695 aa, Vibrio hyugaensis
MTKLSDLLAIEDEAVKQVTLKKMFMPYTEDVCVEGCEKEALTILLNLSSSHQADRCSDWLDLARAKRHLKAAENLEASLDEIKWFHTHNLKFPDCRVKDQRIVAQALTTTEVFISSGVLEQRLGWAHNSAVYRHTLWLLNPFSWQSQPVCILSLIKQESSIWIELLKEFGLSAKSLARLKHTIEEQLPDNHFPDNVSSYSKQLRFPWGDNYISLTPVVSHAIQSELEVRSRNRESKLSFVSSSLPNSASIGNLCGSLGGNMKALNYPLDVKPARGGTLPESRKKSGHYFDDYQVTNTKVCQVLNHLIGSEPSKTQKQRESARKVRSKILRKQIALWMLPLIELRDIVDADPNQQQLEHDDTLAQAFLTQPESDLGSLASEFNRHLHLTFQNNKYAAKFAYHPKLMQLVKAQIVWILEQLSKPTGNADKVTGEQYIYLSSMKVQDAVAMSSPYLCGAPSLTAIWGFMHRYQREFNKLVNCNSLFEFSSFSFYVRSEKIQPTAKLTEPNSVAKARTVSNAKRPTIRSERLADLEIDLVIRVHSDSRISDFKAALKTALPVAFAGGALYQPQLSTQVEWLKTFTSRSELFHVIKGLPAYGRWLYPSESQPSNFDELERLITKDADNLPVSIGYHLLECPTKRCNSITDCHAYAENAIGLAKKVNPIEVRFSGRDHFFNHAFWSIECSSETILIKNYRD

SEQ ID NO: 410 - protein, 732 aa, synthetic construct
MPKKKRKVGSGDYKDDDDKDYKDDDDKDYKDDDDKGSGTKLSDLLAIEDEAVKQVTLKKMFMPYTEDVCVEGCEKEALTILLNLSSSHQADRCSDWLDLARAKRHLKAAENLEASLDEIKWFHTHNLKFPDCRVKDQRIVAQALTTTEVFISSGVLEQRLGWAHNSAVYRHTLWLLNPFSWQSQPVCILSLIKQESSIWIELLKEFGLSAKSLARLKHTIEEQLPDNHFPDNVSSYSKQLRFPWGDNYISLTPVVSHAIQSELEVRSRNRESKLSFVSSSLPNSASIGNLCGSLGGNMKALNYPLDVKPARGGTLPESRKKSGHYFDDYQVTNTKVCQVLNHLIGSEPSKTQKQRESARKVRSKILRKQIALWMLPLIELRDIVDADPNQQQLEHDDTLAQAFLTQPESDLGSLASEFNRHLHLTFQNNKYAAKFAYHPKLMQLVKAQIVWILEQLSKPTGNADKVTGEQYIYLSSMKVQDAVAMSSPYLCGAPSLTAIWGFMHRYQREFNKLVNCNSLFEFSSFSFYVRSEKIQPTAKLTEPNSVAKARTVSNAKRPTIRSERLADLEIDLVIRVHSDSRISDFKAALKTALPVAFAGGALYQPQLSTQVEWLKTFTSRSELFHVIKGLPAYGRWLYPSESQPSNFDELERLITKDADNLPVSIGYHLLECPTKRCNSITDCHAYAENAIGLAKKVNPIEVRFSGRDHFFNHAFWSIECSSETILIKNYRD

SEQ ID NO: 411 - protein, 343 aa, Vibrio hyugaensis
MELCTQLNYVRSLSAGKAYFYYLSKSGEMCPLEIDRTRLRAPKGGYAEAYKGSKFVEKNVAPQDLAYSNPQFIEECYVKPGVDDIYCAFPLRIRANSLTPDTCSDDEVRSKLSLLANTYKELNGYQELAHRYAKNILLGTWLWRNRECRQLSIEVTTSDSQTLIEENATRLSWYGHWDEASAECLEKLTAYLMRALSDPTEYFYMDVKAKIGVGWGDEVYPSQEFLDDQENGAPTKQLATVELLNGKETAAFHGQKIGAALQSIDDWWHEEADKPLRVNEYGADREYVIARRHVSYGNDFYQLVRNTENWIETMTASQTIPNDVHFIMSVLIKGGLFNCSKAK

SEQ ID NO: 412 - protein, 353 aa, synthetic construct
MPKKKRKVGSGELCTQLNYVRSLSAGKAYFYYLSKSGEMCPLEIDRTRLRAPKGGYAEAYKGSKFVEKNVAPQDLAYSNPQFIEECYVKPGVDDIYCAFPLRIRANSLTPDTCSDDEVRSKLSLLANTYKELNGYQELAHRYAKNILLGTWLWRNRECRQLSIEVTTSDSQTLIEENATRLSWYGHWDEASAECLEKLTAYLMRALSDPTEYFYMDVKAKIGVGWGDEVYPSQEFLDDQENGAPTKQLATVELLNGKETAAFHGQKIGAALQSIDDWWHEEADKPLRVNEYGADREYVIARRHVSYGNDFYQLVRNTENWIETMTASQTIPNDVHFIMSVLIKGGLFNCSKAK

SEQ ID NO: 413 - protein, 201 aa, Vibrio hyugaensis
MTKRYYFCIRYTPVQADYELLAGRCISQMHLFMVNNRQSINKIGVSFPDWSDVTVGQTIAFVAEDKEMMIGLSFQPYFSLMVNEGLFEISSVCEVPDNAIEVRFTRNQTIGKSFLGSKKRRIKRSMARAELSGVEPSLPATNEERVVDSFHRIPISSASSGEDYILFLQKELVGERGAANFNSYGLATNQERKGTVPELRF

SEQ ID NO: 414 - protein, 211 aa, synthetic construct
MPKKKRKVGSGTKRYYFCIRYTPVQADYELLAGRCISQMHLFMVNNRQSINKIGVSFPDWSDVTVGQTIAFVAEDKEMMIGLSFQPYFSLMVNEGLFEISSVCEVPDNAIEVRFTRNQTIGKSFLGSKKRRIKRSMARAELSGVEPSLPATNEERVVDSFHRIPISSASSGEDYILFLQKELVGERGAANFNSYGLATNQERKGTVPELRF

SEQ ID NO: 415 - protein, 695 aa, Vibrio crassostreae
MTKLSDLLTIEDEAVKQSALKKMFMPYTEDVCVEGCEKEALTILLNLSSSHQADRCSDWLDVARAKRHLKAAENLEASLDEIKWFHTHNLKFPDCRVKDQRIIAQPLVTTEAFISNAVLEQRLGWAHNSAVYRHTLWLLNPFRWQSQSVSLLSLVQQETSVWVELLKEFGLGIKSLARLKHTIEEQLPENSFPDSVSTYSKQLRFPWGDDYVSVTPVVSHAIQRELEVRSRSRESKLSFVSSSLPNSASIGNLCGSLGGHMKVLNYPLDVKPAQGGTLTESRKKSGHYFDDYQVTNAKICQVLNHLIGSEPSKTQKQRESARKVRSKILRKQIALWMLPLIELRDIVDADPNQQQLEHDGSLVQSFLALPESDLGSLASEFNRRLHLTFQNNKYAAKFAYHPKLMQVVKAQIVWILEQLSKPNGNEDKVTGEQYIYLSSMRVQDAVAMSSPYLCGAPSLAAIWGFMHHYQREFNKLVNCDSPFEFSSFSFYVRSENIQSIAKLTEPNSVAKARTVSNAKRPTIRSERLADLEIDLVIRIHSDSRISDFKSALKTALPVAFAGGALYQPQLSTQIEWLRTFTSRSELFHVLKGLPAYGRWLYPSENQSSDFDDLEHLITKDADNLPVSIGYHLLERPTKRDNSITSCHAYAENVIGLALRVSPIEVRFSGRDHFLNHAFWSIECSSETILIKNYRD

SEQ ID NO: 416 - protein, 732 aa, synthetic construct
MPKKKRKVGSGDYKDDDDKDYKDDDDKDYKDDDDKGSGTKLSDLLTIEDEAVKQSALKKMFMPYTEDVCVEGCEKEALTILLNLSSSHQADRCSDWLDVARAKRHLKAAENLEASLDEIKWFHTHNLKFPDCRVKDQRIIAQPLVTTEAFISNAVLEQRLGWAHNSAVYRHTLWLLNPFRWQSQSVSLLSLVQQETSVWVELLKEFGLGIKSLARLKHTIEEQLPENSFPDSVSTYSKQLRFPWGDDYVSVTPVVSHAIQRELEVRSRSRESKLSFVSSSLPNSASIGNLCGSLGGHMKVLNYPLDVKPAQGGTLTESRKKSGHYFDDYQVTNAKICQVLNHLIGSEPSKTQKQRESARKVRSKILRKQIALWMLPLIELRDIVDADPNQQQLEHDGSLVQSFLALPESDLGSLASEFNRRLHLTFQNNKYAAKFAYHPKLMQVVKAQIVWILEQLSKPNGNEDKVTGEQYIYLSSMRVQDAVAMSSPYLCGAPSLAAIWGFMHHYQREFNKLVNCDSPFEFSSFSFYVRSENIQSIAKLTEPNSVAKARTVSNAKRPTIRSERLADLEIDLVIRIHSDSRISDFKSALKTALPVAFAGGALYQPQLSTQIEWLRTFTSRSELFHVLKGLPAYGRWLYPSENQSSDFDDLEHLITKDADNLPVSIGYHLLERPTKRDNSITSCHAYAENVIGLALRVSPIEVRFSGRDHFLNHAFWSIECSSETILIKNYRD

SEQ ID NO: 417 - protein, 343 aa, Vibrio crassostreae
MELCTQLNYVRSLSAGKAYFYYLSKSGEMCPLEIDRTRLRAPKGGYAEAYKGGKFVGKNVAPQDLAYSNPQFIEECYVKPGVDDIYCAFPLRIRANSLTPDTCSDDEVRSKLSLLAKTYEELNGYQELALRYAKNILLGRWLWRNRECRKLSIEVTTSDSQILIVENATRLSWYGHWGEASEECLEKLTAYLMRALSDPTEYFYMDVKAKIGVGWGDEVYPSQEFLGSREDGVPTKQLATVELLNGKETVAFHGQKVGAALQSIDDWWHENADKPLRVNEYGADREYVIARRHVSYGNDFYQLVRNTENWIETMTASQTIPNDVHFIMSVLIKGGLFNCSKAK

SEQ ID NO: 418 - protein, 353 aa, synthetic construct
MPKKKRKVGSGELCTQLNYVRSLSAGKAYFYYLSKSGEMCPLEIDRTRLRAPKGGYAEAYKGGKFVGKNVAPQDLAYSNPQFIEECYVKPGVDDIYCAFPLRIRANSLTPDTCSDDEVRSKLSLLAKTYEELNGYQELALRYAKNILLGRWLWRNRECRKLSIEVTTSDSQILIVENATRLSWYGHWGEASEECLEKLTAYLMRALSDPTEYFYMDVKAKIGVGWGDEVYPSQEFLGSREDGVPTKQLATVELLNGKETVAFHGQKVGAALQSIDDWWHENADKPLRVNEYGADREYVIARRHVSYGNDFYQLVRNTENWIETMTASQTIPNDVHFIMSVLIKGGLFNCSKAK

SEQ ID NO: 419 - protein, 201 aa, Vibrio crassostreae
MTTRYYFCIRYTPVQADYELLAGRCISQMHLFMVNNRQAINKIGVSFPDWSDVTVGQTIAFVAEDKEMMVGLSFQPYFSVMVNEGLFEISSVCEVPDTAVEVRFTRNQTIGKSFLGSKKRRIKRSMARAELSGVESSLPVTNEERVIDSFHRIPISSGSSAQDYILFVQKESVGERVAANFNSYGLATNQESRGTVPDLRF

SEQ ID NO: 420 - protein, 211 aa, synthetic construct
MPKKKRKVGSGTTRYYFCIRYTPVQADYELLAGRCISQMHLFMVNNRQAINKIGVSFPDWSDVTVGQTIAFVAEDKEMMVGLSFQPYFSVMVNEGLFEISSVCEVPDTAVEVRFTRNQTIGKSFLGSKKRRIKRSMARAELSGVESSLPVTNEERVIDSFHRIPISSGSSAQDYILFVQKESVGERVAANFNSYGLATNQESRGTVPDLRF

SEQ ID NO: 421 - protein, 706 aa, Aeromonas salmonicida
MQLREWFNTSDKAERDKALRRAFVPFTPDIEIAGDEWLALVVLLNLTLKRGQGDELTDKRHAKALLLDQKHLEKCVKQVRWLHSHNLKYPDSRVSHQRLVIASPPQIPGVVTSAGLPMRLGWANNSADINHAKLFCSSFLYHGVTTNLALQLATDVPAPAWTTAFRKLGLADSAIAALQSQLAQLLATSTVPAEVSPYSKQVRFWYQGDYCAITPVVSHGLMSQLHQLIYEKRIPHLIISHDHPASVGSLVGAVGGKIAVLHYPPPVSVEKRRNFSQSRATRINQGDSLFDRTILRDQIFIHALEHLIAPSGLTRRQRKQSHLSALRYLRRQLACWIAPLIEWRDEVEQNQGALPSIDPSRVEWQVLSCPQSELPSLGIALAESCHLALQSHPATRRLAFHPRLLMPIKTQLRWLLNKLALDESVPPQTATCCYLHLSGLRVYDAVALANPYLCGIPSLSALAGFCHDYERRLTAVLKRSVRLTGVAWYLRDCHLQPAKNLPEPSSPLSAHEVSAIRRPGLIDSKHCDLGMDLVLALHVDADHPAFSADEQNLLQAAFPSRFAGGCLHPPSLYEGQPWCNIYTNRGALFSTLSRLPRSGCWVYPHLSQVTDLEDFFETFSTDRRLRPISAGYVFLEPPQLRAGSVEKHHAYAESALGLALCINPVEMRLTGNNHFFKHGFWQLNVSNGAMLMTGVGNREPPHRGTM

SEQ ID NO: 422 - protein, 743 aa, synthetic construct
MPKKKRKVGSGDYKDDDDKDYKDDDDKDYKDDDDKGSGQLREWFNTSDKAERDKALRRAFVPFTPDIEIAGDEWLALVVLLNLTLKRGQGDELTDKRHAKALLLDQKHLEKCVKQVRWLHSHNLKYPDSRVSHQRLVIASPPQIPGVVTSAGLPMRLGWANNSADINHAKLFCSSFLYHGVTTNLALQLATDVPAPAWTTAFRKLGLADSAIAALQSQLAQLLATSTVPAEVSPYSKQVRFWYQGDYCAITPVVSHGLMSQLHQLIYEKRIPHLIISHDHPASVGSLVGAVGGKIAVLHYPPPVSVEKRRNFSQSRATRINQGDSLFDRTILRDQIFIHALEHLIAPSGLTRRQRKQSHLSALRYLRRQLACWIAPLIEWRDEVEQNQGALPSIDPSRVEWQVLSCPQSELPSLGIALAESCHLALQSHPATRRLAFHPRLLMPIKTQLRWLLNKLALDESVPPQTATCCYLHLSGLRVYDAVALANPYLCGIPSLSALAGFCHDYERRLTAVLKRSVRLTGVAWYLRDCHLQPAKNLPEPSSPLSAHEVSAIRRPGLIDSKHCDLGMDLVLALHVDADHPAFSADEQNLLQAAFPSRFAGGCLHPPSLYEGQPWCNIYTNRGALFSTLSRLPRSGCWVYPHLSQVTDLEDFFETFSTDRRLRPISAGYVFLEPPQLRAGSVEKHHAYAESALGLALCINPVEMRLTGNNHFFKHGFWQLNVSNGAMLMTGVGNREPPHRGTM

SEQ ID NO: 423 - protein, 339 aa, Aeromonas salmonicida
MSYSRSLSPGKAVFFYTTPECDFVPLRVEVARVLGQKCGFSEGFDAHFQPKTLERHELAYGNPQTIEVCYVPPNVHEIYCRFSLRVKANALGPTVCSDSEVMQTLVNLSRCYQDRGGFIELARRYSRNLIMATWLWRNRQSQGTRIEIHTSQGSRYMIDDVRHLDWQGQWPASAQEQWLQLADEMATALTRPDLFWFADVTAVMKTAFCQEIYPSQAFTERPDNHTEPSKKLATVECTDGQLAACLTAQKLGAALQKIDDWWGEEVDEPLRVHEYAADPKHQTSMRHPVSGLDFYHLLSRTDELVAQMESSPESSDIHRDIHYLMAVLVKGGLFQKGRS

SEQ ID NO: 424 - protein, 349 aa, synthetic construct
MPKKKRKVGSGSYSRSLSPGKAVFFYTTPECDFVPLRVEVARVLGQKCGFSEGFDAHFQPKTLERHELAYGNPQTIEVCYVPPNVHEIYCRFSLRVKANALGPTVCSDSEVMQTLVNLSRCYQDRGGFIELARRYSRNLIMATWLWRNRQSQGTRIEIHTSQGSRYMIDDVRHLDWQGQWPASAQEQWLQLADEMATALTRPDLFWFADVTAVMKTAFCQEIYPSQAFTERPDNHTEPSKKLATVECTDGQLAACLTAQKLGAALQKIDDWWGEEVDEPLRVHEYAADPKHQTSMRHPVSGLDFYHLLSRTDELVAQMESSPESSDIHRDIHYLMAVLVKGGLFQKGRS

SEQ ID NO: 425 - protein, 207 aa, Aeromonas salmonicida
MNNERFFFVVRYLPSRADSALLAGRCISQLHGYLLRNSHVQIGVSFPDWSDTQLGSYIGFVSAEKDHLDHFRQRAYFQIMQEDGLFSLTTTLEVPIGCAEVRFVRNQGLAKLFAGERRRRLARAKRRAEARGDVFLPQSPPEHRDVLQFHRVLMQSQSNNQDFVMHIEKEPYDNSDSNTGFNNYGLACRVQHRGSVPELASIVATLF

SEQ ID NO: 426 - protein, 217 aa, synthetic construct
MPKKKRKVGSGNNERFFFVVRYLPSRADSALLAGRCISQLHGYLLRNSHVQIGVSFPDWSDTQLGSYIGFVSAEKDHLDHFRQRAYFQIMQEDGLFSLTTTLEVPIGCAEVRFVRNQGLAKLFAGERRRRLARAKRRAEARGDVFLPQSPPEHRDVLQFHRVLMQSQSNNQDFVMHIEKEPYDNSDSNTGFNNYGLACRVQHRGSVPELASIVATLF

SEQ ID NO: 427 - protein, 688 aa, Klebsiella oxytoca
MHIRELLKIKDHSERDRALRHGFSPIREKIDMEGFEYETLVVLLNMTLKRDLVHNLFDVRLARQLLFDKNHLAHCVNAVRWLHTHNLKYPDSRVRGQRLIICSPAVIPGIVSSADLPQEMGWANNGADINFARLFCSFFRHNGSITCLAKLLTEGCSGIVKALERLGTSTDDICLLRVAIANNISESVIPSDVSIYSRQLRGFLQGKDVAITPVVSHALMARLQQLIYQQRKPHIIIRHDHPASMGNLVASTGGNIAVMYYPPLVSVHKERSFIHSRVGLLQEREHLFDNNVLREKELFNALQNLVSHNGGSQRQIRQQRLSALRYLRYQLVIWLKPVIECIDALEENREDILSLPESIEKKVLTQSVNRLDELSSELAGHFHLSLQHHPLFRRFAFHSELVVSVESQLKWILKNISRSDPDTPITQSCREFYLHLSGLNIYDASAMSNPYLCGIPSLTALAGFCHDYERRVSALMEQKVCFTEVAWYIGHYNLISGRQLPAAMIPERKNTISSLRRPGITDEKCCDMGIELVIKLQFPEECKLPESGLLYAASPSRFAGGVLHPPSFSGEKSWCQLYSDQDALYSVLSRLPGSGCWIYPVRTTITTLEEMFTELSSDYRLRPVSSGFILLEEMQYRAGSLASQHVYAESALGLARCHNPIEIRLAGKKNFYNQGFWPLDYEDRTIIT

SEQ ID NO: 428 - protein, 725 aa, synthetic construct
MPKKKRKVGSGDYKDDDDKDYKDDDDKDYKDDDDKGSGHIRELLKIKDHSERDRALRHGFSPIREKIDMEGFEYETLVVLLNMTLKRDLVHNLFDVRLARQLLFDKNHLAHCVNAVRWLHTHNLKYPDSRVRGQRLIICSPAVIPGIVSSADLPQEMGWANNGADINFARLFCSFFRHNGSITCLAKLLTEGCSGIVKALERLGTSTDDICLLRVAIANNISESVIPSDVSIYSRQLRGFLQGKDVAITPVVSHALMARLQQLIYQQRKPHIIIRHDHPASMGNLVASTGGNIAVMYYPPLVSVHKERSFIHSRVGLLQEREHLFDNNVLREKELFNALQNLVSHNGGSQRQIRQQRLSALRYLRYQLVIWLKPVIECIDALEENREDILSLPESIEKKVLTQSVNRLDELSSELAGHFHLSLQHHPLFRRFAFHSELVVSVESQLKWILKNISRSDPDTPITQSCREFYLHLSGLNIYDASAMSNPYLCGIPSLTALAGFCHDYERRVSALMEQKVCFTEVAWYIGHYNLISGRQLPAAMIPERKNTISSLRRPGITDEKCCDMGIELVIKLQFPEECKLPESGLLYAASPSRFAGGVLHPPSFSGEKSWCQLYSDQDALYSVLSRLPGSGCWIYPVRTTITTLEEMFTELSSDYRLRPVSSGFILLEEMQYRAGSLASQHVYAESALGLARCHNPIEIRLAGKKNFYNQGFWPLDYEDRTIIT

SEQ ID NO: 429 - protein, 345 aa, Klebsiella oxytoca
MELCTHLSYMRSISPGKAVFYYKRPECEFVPLEIQTSKIRGQKCSYSEGFRENLQPRKLQQHDLAYANPLTIEICYVPADVNEIYCRFTLRIEANSLRPYVCGDPHVLNTLTELALEYKKHDGYKELAKRYSTNLLMGSWLWRNRFTQSTQLEIKTSLNSTYRILDSRELNWSEAWPESEQRQRELLEREIETALSEPGVFWGADVIATLQTSFCQEIYPSQKFIEKTVDYSIASRQLATTECSNGKQAACITAQKIGAALQRIDDWWSADADYPLRVHEYGAEPERLTARRHPVSGHDFYHLLTKADIFLNDFKSKKMKKISGDIHFLMSVLVKGGLFQKGRGA

SEQ ID NO: 430 - protein, 355 aa, synthetic construct
MPKKKRKVGSGELCTHLSYMRSISPGKAVFYYKRPECEFVPLEIQTSKIRGQKCSYSEGFRENLQPRKLQQHDLAYANPLTIEICYVPADVNEIYCRFTLRIEANSLRPYVCGDPHVLNTLTELALEYKKHDGYKELAKRYSTNLLMGSWLWRNRFTQSTQLEIKTSLNSTYRILDSRELNWSEAWPESEQRQRELLEREIETALSEPGVFWGADVIATLQTSFCQEIYPSQKFIEKTVDYSIASRQLATTECSNGKQAACITAQKIGAALQRIDDWWSADADYPLRVHEYGAEPERLTARRHPVSGHDFYHLLTKADIFLNDFKSKKMKKISGDIHFLMSVLVKGGLFQKGRGA

SEQ ID NO: 431 - protein, 206 aa, Klebsiella oxytoca
MRMTRYFFSVYYLPEDADYPLLAGRCISTLHGYTSHHPDTRIGVSFPDWTDTTLGRTIAFVSVNRSHLEQLKERAYFKILKEEKIFSISPVLKVPEYCPDVMFIRNQTIAKCFVKERKRRLERAKRRAEARGEVFQPRVNSPLRSIEAFHGIFMQSISNGCSFLLHIQKKEARIQSNHMYCSYGLASNEVYTGHVPDLSSVVKKLF

SEQ ID NO: 432 - protein, 216 aa, synthetic construct
MPKKKRKVGSGRMTRYFFSVYYLPEDADYPLLAGRCISTLHGYTSHHPDTRIGVSFPDWTDTTLGRTIAFVSVNRSHLEQLKERAYFKILKEEKIFSISPVLKVPEYCPDVMFIRNQTIAKCFVKERKRRLERAKRRAEARGEVFQPRVNSPLRSIEAFHGIFMQSISNGCSFLLHIQKKEARIQSNHMYCSYGLASNEVYTGHVPDLSSVVKKLF

SEQ ID NO: 433 - protein, 708 aa, Pseudoalteromonas arctica
MIKLECICRHGEYMHLKELLEITDIAERDRLIRRAFNPYTTTIDITGCEGNTLIILLNLTYRKNQVDDLLDKQLAKQALKSEEHINKCIKEIAWFHTHNLKYPDIRVSKQNLAVAPPLLDSYVLSSANYPKAYGWSHDSAKVNFAKLFVSYFKWQNQDSCLAQVLATNSDNWKAAFTSLGLSVKAFKSLCVTVKKSLPEEAIPDSVDRYSRQIRMPYHDGYLAVTPVISHVVQSKIQQAAIDKRARFSNVEFTRPAAVSLLAASLGGVVNVLNYPPKILNKYHGLSSSRQFKLNNGQTVFNVGALLKPEFIKALEGIIFSNNALALKQRRQQKVKNIRDVRSTLLEWFSPIYEWRLDIIETEVGLEQLEGTSDQLEYKILSLSDDELPLLTIPLFRLLNEMLSDVSMTQRYAFHPQLMSPLKAALQWLLINLTDQKNELIEEDDEHYRYLHLSGIRVFDAQALSNPYCSGIPSLTAVWGMLHSYQRKLNEALGINVRFTSFSWFIRDYSAVAGKKLPELSLQGAQQNKLKRPGIIDGKYCDLIFDLIIHIDGYEDDLQTVDSEPDILKAYFPSTFAGGVMHQPQLSSNVNWCYLYSNENQLFEKLKRLPLSGCWVMPNDHKIEDLDELLLLLNNDSKLSPSMMGYMLLTEPMARVGALERLHCYAEPAIGVVKYETAISVRLKGIGNYFNSAFWVLDAQEKFMLMKKV

SEQ ID NO: 434 - protein, 745 aa, synthetic construct
MPKKKRKVGSGDYKDDDDKDYKDDDDKDYKDDDDKGSGIKLECICRHGEYMHLKELLEITDIAERDRLIRRAFNPYTTTIDITGCEGNTLIILLNLTYRKNQVDDLLDKQLAKQALKSEEHINKCIKEIAWFHTHNLKYPDIRVSKQNLAVAPPLLDSYVLSSANYPKAYGWSHDSAKVNFAKLFVSYFKWQNQDSCLAQVLATNSDNWKAAFTSLGLSVKAFKSLCVTVKKSLPEEAIPDSVDRYSRQIRMPYHDGYLAVTPVISHVVQSKIQQAAIDKRARFSNVEFTRPAAVSLLAASLGGVVNVLNYPPKILNKYHGLSSSRQFKLNNGQTVFNVGALLKPEFIKALEGIIFSNNALALKQRRQQKVKNIRDVRSTLLEWFSPIYEWRLDIIETEVGLEQLEGTSDQLEYKILSLSDDELPLLTIPLFRLLNEMLSDVSMTQRYAFHPQLMSPLKAALQWLLINLTDQKNELIEEDDEHYRYLHLSGIRVFDAQALSNPYCSGIPSLTAVWGMLHSYQRKLNEALGINVRFTSFSWFIRDYSAVAGKKLPELSLQGAQQNKLKRPGIIDGKYCDLIFDLIIHIDGYEDDLQTVDSEPDILKAYFPSTFAGGVMHQPQLSSNVNWCYLYSNENQLFEKLKRLPLSGCWVMPNDHKIEDLDELLLLLNNDSKLSPSMMGYMLLTEPMARVGALERLHCYAEPAIGVVKYETAISVRLKGIGNYFNSAFWVLDAQEKFMLMKKV

SEQ ID NO: 435 - protein, 350 aa, Pseudoalteromonas arctica
MELCNILKYDRSLYPSKAVFFYKTADSDFVPLEADINKVRGPKSGFTEAFTPQFLPKNISPQDLTHNNILTLEECYVPPNVEHIFCRFSLRVQANSLAPSGCSDPEVFSLLKELATIFKECGGYKELATRYCRNILLGTWLWRNQNTGNTQIEIKTSKGNRYLIDNTRKLAWESKWASDDQRVLEELSNEIESALTDPNVFWSADITAKIEASFCQEVYPSQILNDKVKQGEASKQFVKSKCADGRYAVSFNSVKIGAALQSIDDWWDEDASKRLRVHEFGADKEIGIARRPPDSEQNFYSIFKNTEWYLSALKNCITNKNENIDPAIYYLFSVLIKGGMFQKKAEAKKA

SEQ ID NO: 436 - protein, 360 aa, synthetic construct
MPKKKRKVGSGELCNILKYDRSLYPSKAVFFYKTADSDFVPLEADINKVRGPKSGFTEAFTPQFLPKNISPQDLTHNNILTLEECYVPPNVEHIFCRFSLRVQANSLAPSGCSDPEVFSLLKELATIFKECGGYKELATRYCRNILLGTWLWRNQNTGNTQIEIKTSKGNRYLIDNTRKLAWESKWASDDQRVLEELSNEIESALTDPNVFWSADITAKIEASFCQEVYPSQILNDKVKQGEASKQFVKSKCADGRYAVSFNSVKIGAALQSIDDWWDEDASKRLRVHEFGADKEIGIARRPPDSEQNFYSIFKNTEWYLSALKNCITNKNENIDPAIYYLFSVLIKGGMFQKKAEAKKA

SEQ ID NO: 437 - protein, 203 aa, Pseudoalteromonas arctica
MQRYYFTVHFLPKQANLALLTGRCISIMHGFILKHNIEGMGVTFPAWSDSSIGNVIAFVHKDMEVLNSLKEQAYFVDMQDCGFFKISQISTVPDSCQEVRFIRNQSVAKIFTGESRRRLKRLQKRALARGEDFNPKKLEAPREIDIFHRVAMTSKSSQEDYILHIQKQDADCQAEPVLSNYGFSSNEKFKGTVPDLSPLIESN

SEQ ID NO: 438 - protein, 213 aa, synthetic construct
MPKKKRKVGSGQRYYFTVHFLPKQANLALLTGRCISIMHGFILKHNIEGMGVTFPAWSDSSIGNVIAFVHKDMEVLNSLKEQAYFVDMQDCGFFKISQISTVPDSCQEVRFIRNQSVAKIFTGESRRRLKRLQKRALARGEDFNPKKLEAPREIDIFHRVAMTSKSSQEDYILHIQKQDADCQAEPVLSNYGFSSNEKFKGTVPDLSPLIESN

SEQ ID NO: 439 - protein, 683 aa, Pseudoalteromonas translucida
MNLQDALAIEPLKEKTTALRKLFVPYTSHVEVDGFEELALTVLINLVYKRSEIDDLTSARTAKSVLRDEVLLSKCINEVKWFHTHNLKYPDIRVSHQRLISEVVSEDIAGICSRSLPLSFGWSHNSAEINHAKLFLTSFNWQGEVTCLARLLINEEPVWINLIRAYGFTKKAVLEISGKIKQQLPVAEFPLEVSSFSPQLQMPFQQSYLVVTPVVSHAMLAKIQQLTTDRKLNFALVEHSRPANVGDLASSVGGNIRVLRYFPKTYSKAVNRSKVANNDIEKAFKIRALLSSQFQQALLVLVGIKQFNTLRQKRLARVAAIRQVRVSLQLWLDNILEAKNNAQNQVYPEWVRHYLDQSITNCISQFSNVLNESLGNLSKLKRFAYHPNLMGLFKAQLNYVFTHCAAEQEILNDEQIVYVHCQDMRVFDAEAMANPYIQGMPSLTALNGLAHNFERKLKNFIDPSIKCIGSAIYIENYQLHTGKPLPEPSKLKQVAGRSHVIRSGIIDKPKCDITLDLVFRLFVPNTELLDKLNSQLIKPALPSSFAGGTMHPPSLYQNIDWCHVHTKPSELFKKLKAKSSNGSWLYPSKKVVKSFEQLIDALNSNFNLRPAAIGLAALEEPVKRDAALHEYHCYAEPVIGLLECVSNTSVKYAGAKQFFHDAFWVMDVQKESMLMKKSKFEYE

SEQ ID NO: 440 - protein, 720 aa, synthetic construct
MPKKKRKVGSGDYKDDDDKDYKDDDDKDYKDDDDKGSGNLQDALAIEPLKEKTTALRKLFVPYTSHVEVDGFEELALTVLINLVYKRSEIDDLTSARTAKSVLRDEVLLSKCINEVKWFHTHNLKYPDIRVSHQRLISEVVSEDIAGICSRSLPLSFGWSHNSAEINHAKLFLTSFNWQGEVTCLARLLINEEPVWINLIRAYGFTKKAVLEISGKIKQQLPVAEFPLEVSSFSPQLQMPFQQSYLVVTPVVSHAMLAKIQQLTTDRKLNFALVEHSRPANVGDLASSVGGNIRVLRYFPKTYSKAVNRSKVANNDIEKAFKIRALLSSQFQQALLVLVGIKQFNTLRQKRLARVAAIRQVRVSLQLWLDNILEAKNNAQNQVYPEWVRHYLDQSITNCISQFSNVLNESLGNLSKLKRFAYHPNLMGLFKAQLNYVFTHCAAEQEILNDEQIVYVHCQDMRVFDAEAMANPYIQGMPSLTALNGLAHNFERKLKNFIDPSIKCIGSAIYIENYQLHTGKPLPEPSKLKQVAGRSHVIRSGIIDKPKCDITLDLVFRLFVPNTELLDKLNSQLIKPALPSSFAGGTMHPPSLYQNIDWCHVHTKPSELFKKLKAKSSNGSWLYPSKKVVKSFEQLIDALNSNFNLRPAAIGLAALEEPVKRDAALHEYHCYAEPVIGLLECVSNTSVKYAGAKQFFHDAFWVMDVQKESMLMKKSKFEYE

SEQ ID NO: 441 - protein, 342 aa, Pseudoalteromonas translucida
MQLPRHLSYTRSLSPSKAVFFYKTPESDFEPLQIEQNKLVGQKSGFGDAYQKQNVAKNLAPQDLAFGNPQTIDVCYVPPTVNELFCRFSLRVEANCIEPHVCDDPKVIYWLKRFFETYKKHNGLNEVATRYAKNILMGNWLWRNRQSPNVDIEILTEHAAPIVVEGAQKLKWQGNWQNNQTALLTLSESIQEGLSNPQNYCYLDITAKIKNAFSQEVHPSQKFVDNVEQGMSSKQLAYTQVGDKKAASLNSQKVGAAIQTIDDWYEEGYKPLRTHEYGADKQILVAHRTPKSHSDFYSLLPRIALHIKHMEKHGLEQSEQSNSIHFIAAVLIKGGLFQRSKG

SEQ ID NO: 442 - protein, 352 aa, synthetic construct
MPKKKRKVGSGQLPRHLSYTRSLSPSKAVFFYKTPESDFEPLQIEQNKLVGQKSGFGDAYQKQNVAKNLAPQDLAFGNPQTIDVCYVPPTVNELFCRFSLRVEANCIEPHVCDDPKVIYWLKRFFETYKKHNGLNEVATRYAKNILMGNWLWRNRQSPNVDIEILTEHAAPIVVEGAQKLKWQGNWQNNQTALLTLSESIQEGLSNPQNYCYLDITAKIKNAFSQEVHPSQKFVDNVEQGMSSKQLAYTQVGDKKAASLNSQKVGAAIQTIDDWYEEGYKPLRTHEYGADKQILVAHRTPKSHSDFYSLLPRIALHIKHMEKHGLEQSEQSNSIHFIAAVLIKGGLFQRSKG

SEQ ID NO: 443 - protein, 200 aa, Pseudoalteromonas translucida
MKRYYFTITYLPQSCDVSLLAGRCIGILHGFMSSREISNIGVCFPKWNEQTIGNELAFVSTNKKQLTNLSQQSYFEMMAHDKLFGLSKILEVPVNQSEVMFVRNQSVAKAFVGEKQRRLKRAKKRAEARGEVYNPEYKFEAKDIGHFHSIPVSSKGNGQSYVLHIQKNENAESIKNQFNNYGFATNQIFLGTVPSLNTLL

SEQ ID NO: 444 - protein, 210 aa, synthetic construct
MPKKKRKVGSGKRYYFTITYLPQSCDVSLLAGRCIGILHGFMSSREISNIGVCFPKWNEQTIGNELAFVSTNKKQLTNLSQQSYFEMMAHDKLFGLSKILEVPVNQSEVMFVRNQSVAKAFVGEKQRRLKRAKKRAEARGEVYNPEYKFEAKDIGHFHSIPVSSKGNGQSYVLHIQKNENAESIKNQFNNYGFATNQIFLGTVPSLNTLL

SEQ ID NO: 445 - protein, 701 aa, Shewanella piezotolerans
MVDKLKFQELLDIDDISERNIVLRRAFTAYTVPLDVTGNEAAALTILLNLTYPRKRVDDLLDMRLAKQTLNTDAHVDACIGEVQWLHTHNLKYPDIRVSKQRLIAASPLLHPHVLSSANCINTLGWSHDSAKVNLAKLFSCHFIWQERVCCLATLLADAPKGWKEAFQALGMLVKDFMNLCGRIKASLPNDDTPNHVDKYSIQVRLPYQDGYLAITPVVSHALQAEIQQAAMAKQGRYTNIEFTRPAGVSELSASLGGNVKALNYPPRIENAEHGLSDSWALKVQSGQTVLNQGALSQPRFKRALEGLLSKNFELALKQRRQQKVACMRQIRATLTEWLSPLLEWRLEVEENKVNTSELGCIHGSFEYQFLTTQKENFVELLSPMFSLLNTVLSNSNTLQKYAFHQHLMKPLKNSLKWLLDNLSKESNAVAIDSDEDNQQRYLYLKGIRVFDAQALSNPYCAGIPSLTAVWGMMHNYQRRLNERLGTQLRLTSFSWFIRQYSSLAGKKLPEYGMQGQKENQFRRAGIVDNKHSDLVFDLVVHIDGYEEDLDAIDNSIDAIKASFPATFAGGVMHPPEIGSVDEWCELYCSEASLYSKLRRLPASGKWIMPTRYQMDSLDGLLQLLKLNVALCPVMSGYLMLGSAESRNYSLEPLHCYAEPAIGVVECATAIDIRLQGMSNFFRRAFWMLDIKETSMLMKRI

SEQ ID NO: 446 - protein, 738 aa, synthetic construct
MPKKKRKVGSGDYKDDDDKDYKDDDDKDYKDDDDKGSGVDKLKFQELLDIDDISERNIVLRRAFTAYTVPLDVTGNEAAALTILLNLTYPRKRVDDLLDMRLAKQTLNTDAHVDACIGEVQWLHTHNLKYPDIRVSKQRLIAASPLLHPHVLSSANCINTLGWSHDSAKVNLAKLFSCHFIWQERVCCLATLLADAPKGWKEAFQALGMLVKDFMNLCGRIKASLPNDDTPNHVDKYSIQVRLPYQDGYLAITPVVSHALQAEIQQAAMAKQGRYTNIEFTRPAGVSELSASLGGNVKALNYPPRIENAEHGLSDSWALKVQSGQTVLNQGALSQPRFKRALEGLLSKNFELALKQRRQQKVACMRQIRATLTEWLSPLLEWRLEVEENKVNTSELGCIHGSFEYQFLTTQKENFVELLSPMFSLLNTVLSNSNTLQKYAFHQHLMKPLKNSLKWLLDNLSKESNAVAIDSDEDNQQRYLYLKGIRVFDAQALSNPYCAGIPSLTAVWGMMHNYQRRLNERLGTQLRLTSFSWFIRQYSSLAGKKLPEYGMQGQKENQFRRAGIVDNKHSDLVFDLVVHIDGYEEDLDAIDNSIDAIKASFPATFAGGVMHPPEIGSVDEWCELYCSEASLYSKLRRLPASGKWIMPTRYQMDSLDGLLQLLKLNVALCPVMSGYLMLGSAESRNYSLEPLHCYAEPAIGVVECATAIDIRLQGMSNFFRRAFWMLDIKETSMLMKRI

SEQ ID NO: 447 - protein, 370 aa, Shewanella piezotolerans
MELCNVLKYDRSLYPSKAVFFYKTAESNFVPLEAEINRIRGQKAGFTEAFTPQFKSKNLAPQDLAHCNPLILEECYVPPNVEHIYCRFSLRVQANSLKPAGCSEPTVFALLEEFAATFKACGGYKELATRYCKNVLLGTWLWRNQNTGNSQIEIKTSSGNCYQIANTRQLAWDSSWPADAQQVLEELSHEVHQALTDPAVFWHAKITAKIETAFCQEIYPSQSFGEKAAQGEASKQFAKVKCVDGRYAVSFNSVKIGAALQLIDDWWDVDGSKRLRIHEYGADKEIGVARRAPESKQSFYSLFVNAELYLAELKQQLAEGEYSISPNIYYLFAVLIKGGMFQKKAEAKSKSKAEPTTAKTTTSKATPVKA

SEQ ID NO: 448 - protein, 380 aa, synthetic construct
MPKKKRKVGSGELCNVLKYDRSLYPSKAVFFYKTAESNFVPLEAEINRIRGQKAGFTEAFTPQFKSKNLAPQDLAHCNPLILEECYVPPNVEHIYCRFSLRVQANSLKPAGCSEPTVFALLEEFAATFKACGGYKELATRYCKNVLLGTWLWRNQNTGNSQIEIKTSSGNCYQIANTRQLAWDSSWPADAQQVLEELSHEVHQALTDPAVFWHAKITAKIETAFCQEIYPSQSFGEKAAQGEASKQFAKVKCVDGRYAVSFNSVKIGAALQLIDDWWDVDGSKRLRIHEYGADKEIGVARRAPESKQSFYSLFVNAELYLAELKQQLAEGEYSISPNIYYLFAVLIKGGMFQKKAEAKSKSKAEPTTAKTTTSKATPVKA

SEQ ID NO: 449 - protein, 199 aa, Shewanella piezotolerans
MQRYYFMVRFLPEQANLALLTGRCISVMHGFICKHEIQGLGVSFPAWSDVSIGNMIAFVHTDIAVLNELRLQGYFQDMQEYGAFNIGDVEAVPDSCTEVRFKRNQAIAKMFVGETRRRLKRLEKRALARGEVFNPSKSYEPRELDSFHCIAVGSTSTEQDFLLHVQKENVQKREGAEFSQLGLATNQLLRGTVPEFDMF

SEQ ID NO: 450 - protein, 209 aa, synthetic construct
MPKKKRKVGSGQRYYFMVRFLPEQANLALLTGRCISVMHGFICKHEIQGLGVSFPAWSDVSIGNMIAFVHTDIAVLNELRLQGYFQDMQEYGAFNIGDVEAVPDSCTEVRFKRNQAIAKMFVGETRRRLKRLEKRALARGEVFNPSKSYEPRELDSFHCIAVGSTSTEQDFLLHVQKENVQKREGAEFSQLGLATNQLLRGTVPEFDMF

SEQ ID NO: 451 - protein, 695 aa, Vibrio azureus
MTKLSDLLAIEDEVLKQATLKKMFMPYTEDVCVEGFEKEALTILLNLSSNHQADKCADWLDDARAKNYLNDSKNLKSSLDEIQWFHTHNLKFPDCRVKDSRIIAKPLITSESFISSAALEESWGWSHNSAVYRFTLWLLTPFRWQSQSVNLLSMIKSSNHTWMVLLQDFGLGVEQLADIKELSYIEMPEESFPNRVSEYSKQIRLPRKGHYLTITPVVSHSIQRELEIRSRNKESQLRFISSYLPNPASIGGLCGSLGGYIKILDYSLGIKADSKQTLIRYHQKRSRFFDDYQLTNNKICQTLNRLIGFEPLKTHKQRNASRRIQTKLLRKQIALWMLPLIELRDLQDAEPNQQKMEYQDSLAQAFLAKPELEFTSLVNDFNQRLHLAFQENKFTTQFAYHPKLMQAAKAQIKWVLTQLSKTEQQEDTSHTEQYIYLSSLRVQDVVAMSCPYLSGFPSLTAIWGFVHQYQREFNKRIDSENHVEFSGFSLFVRSEYIQSSAKLSEPNSVATKRTISNVKRPTTLGQRQSDLEMDLVIRVDSKNRLSDYLSELKATFPLVFAGGAVYQPLMSLQIEWLKVFSSKSSFFNRIKGLPANGRWVLPSDEQPNCFDDLEQLLNQDMDNMPISIGFHLLEPPKARENALTEFHAYAENALGIAKRLSPIDVRFAGRDHFFNHAFWSLELTDETILIKNLRD

SEQ ID NO: 452 - protein, 732 aa, synthetic construct
MPKKKRKVGSGDYKDDDDKDYKDDDDKDYKDDDDKGSGTKLSDLLAIEDEVLKQATLKKMFMPYTEDVCVEGFEKEALTILLNLSSNHQADKCADWLDDARAKNYLNDSKNLKSSLDEIQWFHTHNLKFPDCRVKDSRIIAKPLITSESFISSAALEESWGWSHNSAVYRFTLWLLTPFRWQSQSVNLLSMIKSSNHTWMVLLQDFGLGVEQLADIKELSYIEMPEESFPNRVSEYSKQIRLPRKGHYLTITPVVSHSIQRELEIRSRNKESQLRFISSYLPNPASIGGLCGSLGGYIKILDYSLGIKADSKQTLIRYHQKRSRFFDDYQLTNNKICQTLNRLIGFEPLKTHKQRNASRRIQTKLLRKQIALWMLPLIELRDLQDAEPNQQKMEYQDSLAQAFLAKPELEFTSLVNDFNQRLHLAFQENKFTTQFAYHPKLMQAAKAQIKWVLTQLSKTEQQEDTSHTEQYIYLSSLRVQDVVAMSCPYLSGFPSLTAIWGFVHQYQREFNKRIDSENHVEFSGFSLFVRSEYIQSSAKLSEPNSVATKRTISNVKRPTTLGQRQSDLEMDLVIRVDSKNRLSDYLSELKATFPLVFAGGAVYQPLMSLQIEWLKVFSSKSSFFNRIKGLPANGRWVLPSDEQPNCFDDLEQLLNQDMDNMPISIGFHLLEPPKARENALTEFHAYAENALGIAKRLSPIDVRFAGRDHFFNHAFWSLELTDETILIKNLRD

SEQ ID NO: 453 - protein, 346 aa, Vibrio azureus
MRLCNQLNYLRSLSTGKAYFYSLSSDGTINPIGLDRTRLRAPKGGYSEAYQGNNFSPKNVAPQDLAYANPQFIEECYVRPGVDEIYCAFSLRISANSLTPQICNDDDVRTQLSQLARVYKELGGYSELANRYAKNILLGTWLWRNRGPRNIKIEVRTSDSDLFVIDNALRLSWYGQWDNKSSECLKKLTDYFARALSEPTEYFYLDVKAEITVGWGDEIYPSQKFLDTKEHDMPTKQFATIELESGQQTVALHGQKVGAALQLIDDWWHEEADKPLRVNEYGADREYVIARRHPKFKNDFYHLIQNTEAWVEDMVVSQTIPNEVHFIMSILVKGGLFNGSSPKKDK

SEQ ID NO: 454 - protein, 356 aa, synthetic construct
MPKKKRKVGSGRLCNQLNYLRSLSTGKAYFYSLSSDGTINPIGLDRTRLRAPKGGYSEAYQGNNFSPKNVAPQDLAYANPQFIEECYVRPGVDEIYCAFSLRISANSLTPQICNDDDVRTQLSQLARVYKELGGYSELANRYAKNILLGTWLWRNRGPRNIKIEVRTSDSDLFVIDNALRLSWYGQWDNKSSECLKKLTDYFARALSEPTEYFYLDVKAEITVGWGDEIYPSQKFLDTKEHDMPTKQFATIELESGQQTVALHGQKVGAALQLIDDWWHEEADKPLRVNEYGADREYVIARRHPKFKNDFYHLIQNTEAWVEDMVVSQTIPNEVHFIMSILVKGGLFNGSSPKKDK

SEQ ID NO: 455 - protein, 201 aa, Vibrio azureus
MTKRYYFSVKYLPAGADHDLLAGRCIHEMHLFMINNPQAMNKIGVTFPDWGFTSVGQRIAFVAESKEMLTALSFQNYFSLMVSDGLFELSGVLEVPKTVRELRFVRNQSIGKSFRGSKLRRMKRSIARASALGHALKIPQAREERSIEHFHRVPISSGSSGQTYFLFTQKQVVNERSEANFSSYGLATAQERRGTVPDLDL

SEQ ID NO: 456 - protein, 211 aa, synthetic construct
MPKKKRKVGSGTKRYYFSVKYLPAGADHDLLAGRCIHEMHLFMINNPQAMNKIGVTFPDWGFTSVGQRIAFVAESKEMLTALSFQNYFSLMVSDGLFELSGVLEVPKTVRELRFVRNQSIGKSFRGSKLRRMKRSIARASALGHALKIPQAREERSIEHFHRVPISSGSSGQTYFLFTQKQVVNERSEANFSSYGLATAQERRGTVPDLDL

SEQ ID NO: 457 - protein, 696 aa, Vibrio fluvialis
MTTLQQLIEIDDDKLRFSELKKAFMPYTRPIEIDGNEKQALTILLNLSLGKPVAKDSLDISRAERYFADPENLAKAEQEIQWFHTHNLKFPDCRVAEQRILATPLPSETPTLTSQSLEQAYGWAHNSAVYKHTVWSLNTFLWRGKTENVLSLIRLGDEFWQALLAEFGFTPTGQFQFKTLVERQLPGTHFPEEVSRYSKQVRFPWRNDYLSVTPVVSHAMQQELAVLSRHRECSLRFKSMNYPNSASIGNLCGSLAGHINVLNYPVDVVPDSYQTLAASRERTSRYFDDYQLTSKRTCDVLAHLAGFEQLKSRKAQKHVRQYQLKIIRKQIARWLLPLIELRDNLVTEPLGINYEFDDQLAKQFLTIKEDDFLDWTTSLNQRLNLALQNNRFSSRFAYHPKLMRVLKTELIWVLTQLSRPEPGLPNISNDSVQYIYLSSMRAFDVAALSCPYLSGAPSMTAIWGFIHRYQKELEAQMSDEQCRISFNEFAFFIRHESVQTSAKLTEPSVLAKAREVSPVKRTTIIREDYADLVFDLVIRVESNQRISDYHDQLKAALPTNFAGGTLLQPEIDLNIPWLRTYTTKSELFQVVKGLPGYGTWLSPYSYQPQNLTELENTLAKDASLIPIVNGFHLLEKPINRKNGLTNRHAYAENNIALAKRVNPIEVRFGGRDHFFEQAFWSLDVTEQTILIKNLRN

SEQ ID NO: 458 - protein, 733 aa, synthetic construct
MPKKKRKVGSGDYKDDDDKDYKDDDDKDYKDDDDKGSGTTLQQLIEIDDDKLRFSELKKAFMPYTRPIEIDGNEKQALTILLNLSLGKPVAKDSLDISRAERYFADPENLAKAEQEIQWFHTHNLKFPDCRVAEQRILATPLPSETPTLTSQSLEQAYGWAHNSAVYKHTVWSLNTFLWRGKTENVLSLIRLGDEFWQALLAEFGFTPTGQFQFKTLVERQLPGTHFPEEVSRYSKQVRFPWRNDYLSVTPVVSHAMQQELAVLSRHRECSLRFKSMNYPNSASIGNLCGSLAGHINVLNYPVDVVPDSYQTLAASRERTSRYFDDYQLTSKRTCDVLAHLAGFEQLKSRKAQKHVRQYQLKIIRKQIARWLLPLIELRDNLVTEPLGINYEFDDQLAKQFLTIKEDDFLDWTTSLNQRLNLALQNNRFSSRFAYHPKLMRVLKTELIWVLTQLSRPEPGLPNISNDSVQYIYLSSMRAFDVAALSCPYLSGAPSMTAIWGFIHRYQKELEAQMSDEQCRISFNEFAFFIRHESVQTSAKLTEPSVLAKAREVSPVKRTTIIREDYADLVFDLVIRVESNQRISDYHDQLKAALPTNFAGGTLLQPEIDLNIPWLRTYTTKSELFQVVKGLPGYGTWLSPYSYQPQNLTELENTLAKDASLIPIVNGFHLLEKPINRKNGLTNRHAYAENNIALAKRVNPIEVRFGGRDHFFEQAFWSLDVTEQTILIKNLRN

SEQ ID NO: 459 - protein, 346 aa, Vibrio fluvialis
MELCSQLNYVRSLSPGKAYFYYLDDNQRMCPLQIDRTHLRAPKSGYAEAYTGNFKAKNVAPQDLAFSNPQYIEECYVPPGVDDIYCAFSLRIRANSLFPEVCADAATRETLTGLAETYKELDGYKELAKRYAKNILIATWVWRNRECRNIEIEVKTEKKNWKIADARHLEWYGTWDRKSQSALDGLTDYLEKALSDRSDYFNMDIKAKLTVGWGDEVYPSQEFLDVKESGKPTKQLAKVVLNGEEESAAYHSQKVGAAIQLIDDWWDEEADKPLRVNEYGADKEYVIARRHSSLKRDFYSLISKTEDHIESMRKSNDISNDIHFIMAVLAKGGVFSGASKKSKKEE

SEQ ID NO: 460 - protein, 356 aa, synthetic construct
MPKKKRKVGSGELCSQLNYVRSLSPGKAYFYYLDDNQRMCPLQIDRTHLRAPKSGYAEAYTGNFKAKNVAPQDLAFSNPQYIEECYVPPGVDDIYCAFSLRIRANSLFPEVCADAATRETLTGLAETYKELDGYKELAKRYAKNILIATWVWRNRECRNIEIEVKTEKKNWKIADARHLEWYGTWDRKSQSALDGLTDYLEKALSDRSDYFNMDIKAKLTVGWGDEVYPSQEFLDVKESGKPTKQLAKVVLNGEEESAAYHSQKVGAAIQLIDDWWDEEADKPLRVNEYGADKEYVIARRHSSLKRDFYSLISKTEDHIESMRKSNDISNDIHFIMAVLAKGGVFSGASKKSKKEE

SEQ ID NO: 461 - protein, 203 aa, Vibrio fluvialis
MEPRYYFSIRFIPEHTDNELLAGRCVSNMHGFLSHERNRAFKNSLGVCFPRWSDKTVGNEIAFVSPHESILTGLSYQPYFSTMVNEGLFDISDIKIVPDDVEEVRFVFNKRIQKIFNGSKKRRIKRSMQRAEMQGRIYTPISTEEREFELFHEIPISSQSSGHAFVLHIQRQFPVYPEIGNSFNGYGFAANQRWRGTVPLVTF

SEQ ID NO: 462 - protein, 213 aa, synthetic construct
MPKKKRKVGSGEPRYYFSIRFIPEHTDNELLAGRCVSNMHGFLSHERNRAFKNSLGVCFPRWSDKTVGNEIAFVSPHESILTGLSYQPYFSTMVNEGLFDISDIKIVPDDVEEVRFVFNKRIQKIFNGSKKRRIKRSMQRAEMQGRIYTPISTEEREFELFHEIPISSQSSGHAFVLHIQRQFPVYPEIGNSFNGYGFAANQRWRGTVPLVTF

SEQ ID NO: 463 - protein, 704 aa, Vibrio natriegens
MTTLQDLIDIEDSKLRFIAIKKAFMPYTQPVEIDGNEKQALIVLINLSLSKPEAQDWLDLSRAMGYFANSDNLTTAKREIQWFHTHNLKFPDCRVSEQRIIAMPLYSETPTLTSQSLNRVYGWAHNSTVYKHTIWLLNEFRWRGRVENLLNLIRVGEHFWLELLADIGLKPEVQLQIKELIERQLPSTHFPDEVNRYSKQLRFPWKDEYLSVTPVVSHAIQQQLSVLSRQHSCSFHFKTMNFPHSASIGNLCGSLGGNMDILNYPIGVIANRHQTLGASRSRTNRYFDDFQLTSKRTCGVLAHLTGFEQPQMRKAQKHVRQYQLKIIRRQIALWLLPLIELRDNLVTEPIGFYDESDDELAKRFLTINELDFIVLTTSLNQRLNLALQNNRFASRFAYHPKLMRVLKTELIWVLTQLSRPEPACSATSDSTVQYLYLPSMRVFDAAALSCPYLSGAPSLTAVFGFVHRYQRELRDLLPDKEGKLKFKDFAIFIRDESVQTSAKLTEPSVIAKARGISPVKRTTIIREDCSDLVFDIVITIESDQRLSDYLNQLRAALPTNFAGGTLLQPETSLGIDWLSIFVSESDLFQAVKGLPGYGTWLSPYSFQPQNLMELQERLSNDGSLIPVANGFHFLELPQEREGALTNLHCYAENNIALAKRVSPIEVRIAGRDHFFEQVFWSLEVTEQTILIKKGSNRLWNSAVS

SEQ ID NO: 464 - protein, 741 aa, synthetic construct
MPKKKRKVGSGDYKDDDDKDYKDDDDKDYKDDDDKGSGTTLQDLIDIEDSKLRFIAIKKAFMPYTQPVEIDGNEKQALIVLINLSLSKPEAQDWLDLSRAMGYFANSDNLTTAKREIQWFHTHNLKFPDCRVSEQRIIAMPLYSETPTLTSQSLNRVYGWAHNSTVYKHTIWLLNEFRWRGRVENLLNLIRVGEHFWLELLADIGLKPEVQLQIKELIERQLPSTHFPDEVNRYSKQLRFPWKDEYLSVTPVVSHAIQQQLSVLSRQHSCSFHFKTMNFPHSASIGNLCGSLGGNMDILNYPIGVIANRHQTLGASRSRTNRYFDDFQLTSKRTCGVLAHLTGFEQPQMRKAQKHVRQYQLKIIRRQIALWLLPLIELRDNLVTEPIGFYDESDDELAKRFLTINELDFIVLTTSLNQRLNLALQNNRFASRFAYHPKLMRVLKTELIWVLTQLSRPEPACSATSDSTVQYLYLPSMRVFDAAALSCPYLSGAPSLTAVFGFVHRYQRELRDLLPDKEGKLKFKDFAIFIRDESVQTSAKLTEPSVIAKARGISPVKRTTIIREDCSDLVFDIVITIESDQRLSDYLNQLRAALPTNFAGGTLLQPETSLGIDWLSIFVSESDLFQAVKGLPGYGTWLSPYSFQPQNLMELQERLSNDGSLIPVANGFHFLELPQEREGALTNLHCYAENNIALAKRVSPIEVRIAGRDHFFEQVFWSLEVTEQTILIKKGSNRLWNSAVS

SEQ ID NO: 465 - protein, 345 aa, Vibrio natriegens
MELCSQLNYLRSLSPGKAYFYYLDEDNKMRPLQIDRTHLRAPKSGYSEAFSGNFKSKNIAPQDLSYSNPQFIEECYVPPGVDDIYCAFSLRVRANSLSPEVCVDNEVRDILCNFAALYKELGGYRELARRYAQNILMATWVWRNRECRSIRVEVKTEDKEWVITDARFLDWYGSWDKDSQLALDEFTGYLSQALSDRTSYFNMDIKAKLTVGWGDEVYPSQEFLDVKEAGKPTKQLAKVLVNGAESAAFHSQKIGAAIQLIDDWWDENADKPLRVNEYGADKEYVIARRHSSLKRDFYSLAAKTESYVESMRETNLIPDDVHFIMAVLTKGGVFSGASKKGKKDE

SEQ ID NO: 466 - protein, 355 aa, synthetic construct
MPKKKRKVGSGELCSQLNYLRSLSPGKAYFYYLDEDNKMRPLQIDRTHLRAPKSGYSEAFSGNFKSKNIAPQDLSYSNPQFIEECYVPPGVDDIYCAFSLRVRANSLSPEVCVDNEVRDILCNFAALYKELGGYRELARRYAQNILMATWVWRNRECRSIRVEVKTEDKEWVITDARFLDWYGSWDKDSQLALDEFTGYLSQALSDRTSYFNMDIKAKLTVGWGDEVYPSQEFLDVKEAGKPTKQLAKVLVNGAESAAFHSQKIGAAIQLIDDWWDENADKPLRVNEYGADKEYVIARRHSSLKRDFYSLAAKTESYVESMRETNLIPDDVHFIMAVLTKGGVFSGASKKGKKDE

SEQ ID NO: 467 - protein, 203 aa, Vibrio natriegens
MGSRCYFSIRYVPDYADNELLAGRCISNMHGFLSHERNKPFKNSVGICFPVWNEQTVGNVITFVSTNESILTGLSYQPYFSRMVNENLFEISDIKAVPDDAEEVRFVFNKTIQKIFNGSKKRRIKRAMKRAEEFGHAFTPISVEEREFELFHEIPISSKSSGHDFVLHIQRQYPVVAEIEQHFNGYGFASNQLWQGTVPLISF

SEQ ID NO: 468 - protein, 213 aa, synthetic construct
MPKKKRKVGSGGSRCYFSIRYVPDYADNELLAGRCISNMHGFLSHERNKPFKNSVGICFPVWNEQTVGNVITFVSTNESILTGLSYQPYFSRMVNENLFEISDIKAVPDDAEEVRFVFNKTIQKIFNGSKKRRIKRAMKRAEEFGHAFTPISVEEREFELFHEIPISSKSSGHDFVLHIQRQYPVVAEIEQHFNGYGFASNQLWQGTVPLISF

SEQ ID NO: 469 - protein, 50 aa, Vibrio rotiferianus
STFWKNPFIEGVKVTKDMLRSPPKSIGWEDYYQQNNSRKKKGKGRPDFFD

SEQ ID NO: 470 - protein, 51 aa, Vibrio parahaemolyticus
PTFWKNPFIEGVKVTKEMLRSPPRSIGWEDYYQQNNSRKKKGKGRPDFFDK

SEQ ID NO: 471 - protein, 49 aa, Vibrio chagasii
SDRWENPFEKGVKVTEGMLRSPPKDIGWEDYYHHVTSLNAKRNGGNMFE

SEQ ID NO: 472 - protein, 50 aa, Vibrio cholerae
PNSWKNPFEEGVEVTEDMLRPPPKDIGWEDYLRHSTPRVSKPGRNKNFFE

SEQ ID NO: 473 - protein, 49 aa, Vibrio parahaemolyticus
PITWKNPFDEGVKVTEDMLRPPPKDIGWEDYYHNVKPKNQRRKGGNIFE

SEQ ID NO: 474 - protein, 54 aa, Vibrio parahaemolyticus
SDTVNPFNVSHINDVAIDEPDLDIGWEDFKNKPGEILVGKSSRQFTVGDIFATR

SEQ ID NO: 475 - protein, 34 aa, Vibrio anguillarum
CESFNPFDVDIKQLKIIEPSDDVGWENYLAAKGD

SEQ ID NO: 476 - protein, 34 aa, Vibrio parahaemolyticus
CKSFNPFDTDTKKLKIIEPPEDVGWENYLAAKGD

SEQ ID NO: 477 - protein, 49 aa, Oceanospirillum linum
NDQPFKGKLEQVNFREIEMSSRYIRGDSMYPAHIEPAKLSEFYSLSELL

SEQ ID NO: 478 - protein, 53 aa, Vibrio parahaemolyticus
DKRDSINPFKISVDKLMVNEVIDYATYKYDEESASEVKFDTRFADKISINDLL

SEQ ID NO: 479 - protein, 48 aa, Aliivibrio wodanis
KNPFEMNVADVPIQEVESYSKYNLNAMTDDERLTATKFLDAMSLSSLL

SEQ ID NO: 480 - protein, 48 aa, Vibrio fischeri
KNPFEMNVEDIPIQEVISYSKYNLDEMDDNKRLISTKYSDALPLTVIL

SEQ ID NO: 481 - protein, 49 aa, Vibrio crassostreae
KQNPFECKLTELKLVELKTETSYNKGAQFKEDRLIGRSFTDLLPVHMLL

SEQ ID NO: 482 - protein, 48 aa, Vibrio parahaemolyticus
ANPFACSLSEIKLSQIDTNSMYNTTAIATEDRILAPRFTDDFPLSMLL

SEQ ID NO: 483 - protein, 48 aa, Vibrio cholerae
DNPFDRPLEQLSLHQIDSGSAYHLNAITTEDKIVAPRFTDAIPLSMLL

SEQ ID NO: 484 - protein, 48 aa, Vibrio cholerae; identical to SEQ ID NO: 483

SEQ ID NO: 485 - protein, 48 aa, Halomonas sp.
DNPFKLPLNEVPLCEVSKYASYNRYSTVESDMFVSTQFTPKIPTKVLF

SEQ ID NO: 486 - protein, 48 aa, Agarivorans gilvus
SNPFKMAFEEIPVPKVKEYSKLNHAASTLDEQIIRTQFVDGLPISQLL

SEQ ID NO: 487 - protein, 44 aa, Shewanella piezotolerans
LSSNPFTIKFKEIPIPVLSIPSRYNPNALEEKDEIIDRVFEYIY

SEQ ID NO: 488 - protein, 53 aa, Shewanella sp.
AASVFFNPFLEPLDKVLISEVVKPSRYNPNAMTPDEMLIKREFSAPSTLAQLL

SEQ ID NO: 489 - protein, 47 aa, Vibrio parahaemolyticus
EKLKSKNPFKQDIKDIEIYEVIKNSSYNPNALDPEDMLTDRVFAIVK

SEQ ID NO: 490 - protein, 47 aa, Pseudoalteromonas arctica
DSLKLKNPFKQDIKDIIIYEVTKNSSYNPNALDPEDMLTGRKFAIVK

SEQ ID NO: 491 - protein, 48 aa, Enterovibrio coralii
RNPFELPLNEIKLSEIEHYSGYNPRAMSNDDALTNRMFSENIPLKELL

SEQ ID NO: 492 - protein, 48 aa, Pseudoalteromonas sp.
PNLFTLPVAEITASEVERYSLYKLESAQDEDPFIATKFTDQMPISQLL

SEQ ID NO: 493 - protein, 48 aa, Pseudoalteromonas translucida
PNLFTLPVAEITASEVERYSLYKPESSQDEDPFIATKFTDRMPISQLL

SEQ ID NO: 494 - protein, 48 aa, Klebsiella oxytoca
ENPFYIPLENIKIYEVREYSGYEIDGAGKEDRLIPQQLTDRIPINQLL

SEQ ID NO: 495 - protein, 48 aa, Aeromonas salmonicida
VNPFLQPIDEIKACEVKQYSRYEIDAAGKEEVLNPLQFTDKIPISQLL

SEQ ID NO: 496 - protein, 48 aa, Aeromonas salmonicida
ENPFLQSIDEIKACEVTQYSRYEINESGTEEVLNPLKFTDKIPISQLL

SEQ ID NO: 497 - protein, 51 aa, Vibrio sp.
GENPFDEKVNMIQIQTIQQYTRFELDDKTGRRERFDRAFTALQQIPINKLL

SEQ ID NO: 498 - protein, 48 aa, Aeromonas salmonicida
VTNPFTTELEKLLVPQVIEYEGFIIDPENGEIKFTKQIFKDIPLAALL

SEQ ID NO: 499 - protein, 48 aa, Vibrio diazotrophicus
ISNPFEIELDKLLIPQIIEYEGYLLDSDSGDIKFTHQIFEDIPLTELL

SEQ ID NO: 500 - protein, 48 aa, Vibrio fluvialis
ISNPFEIELDKLLISQIIEYEGYILDSDSGDIKFTHQIFEDIPLTELL

SEQ ID NO: 501 - protein, 48 aa, Vibrio hyugaensis
DINPFTVQIDALTIEQIASYENYVTDAETGELRFVKQVFSKLSIQQLI

SEQ ID NO: 502 - protein, 48 aa, Vibrio natriegens
DINPFTVQIDALTIEQIASYENYVTDAETGELRFVKQVFSKLSIQQLV

SEQ ID NO: 503 - protein, 48 aa, Photobacterium aquae
EINPFSVDINELVIKQIESYEEYVPDAETGELKFVGQIFNALTIKQLL

SEQ ID NO: 504 - protein, 48 aa, Vibrio azureus
EANPFSINADELVIKQIESYEEYVVDAANGELKFVQQIFNELTIEQLL

SEQ ID NO: 505 - protein, 52 aa, Oceanospirillum linum
NDQPFKGKLEQVNFREIEMSSRYIRGDSMYPAHIEPAKLSEFYSLSELLSKS

SEQ ID NO: 506 - protein, 54 aa, Vibrio parahaemolyticus
KRDSINPFKISVDKLMVNEVIDYATYKYDEESASEVKFDTRFADKISINDLLRK

SEQ ID NO: 507 - protein, 51 aa, Aliivibrio wodanis
KNPFEMNVADVPIQEVESYSKYNLNAMTDDERLTATKFLDAMSLSSLLSKT

SEQ ID NO: 508 - protein, 51 aa, Vibrio fischeri
KNPFEMNVEDIPIQEVISYSKYNLDEMDDNKRLISTKYSDALPLTVILSQS

SEQ ID NO: 509 - protein, 55 aa, Vibrio crassostreae
KQNPFECKLTELKLVELKTETSYNKGAQFKEDRLIGRSFTDLLPVHMLLSKTPLK

SEQ ID NO: 510 - protein, 54 aa, Vibrio parahaemolyticus
ANPFACSLSEIKLSQIDTNSMYNTTAIATEDRILAPRFTDDFPLSMLLSKSGVK

SEQ ID NO: 511 - protein, 54 aa, Vibrio cholerae
DNPFDRPLEQLSLHQIDSGSAYHLNAITTEDKIVAPRFTDAIPLSMLLSKNGLK

SEQ ID NO: 512 - protein, 54 aa, Vibrio cholerae; identical to SEQ ID NO: 511

SEQ ID NO: 513 - protein, 51 aa, Halomonas sp.
DNPFKLPLNEVPLCEVSKYASYNRYSTVESDMFVSTQFTPKIPTKVLFSKS

SEQ ID NO: 514 - protein, 52 aa, Agarivorans gilvus
SNPFKMAFEEIPVPKVKEYSKLNHAASTLDEQIIRTQFVDGLPISQLLKKNS

SEQ ID NO: 515 - protein, 44 aa, Shewanella piezotolerans; identical to SEQ ID NO: 487

SEQ ID NO: 516 - protein, 54 aa, Shewanella sp.
ASVFFNPFLEPLDKVLISEVVKPSRYNPNAMTPDEMLIKREFSAPSTLAQLLSK

SEQ ID NO: 517 - protein, 46 aa, Vibrio parahaemolyticus
KLKSKNPFKQDIKDIEIYEVIKNSSYNPNALDPEDMLTDRVFAIVK

SEQ ID NO: 518 - protein, 46 aa, Pseudoalteromonas arctica
SLKLKNPFKQDIKDIIIYEVTKNSSYNPNALDPEDMLTGRKFAIVK

SEQ ID NO: 519 - protein, 52 aa, Enterovibrio coralii
RNPFELPLNEIKLSEIEHYSGYNPRAMSNDDALTNRMFSENIPLKELLKKKG

SEQ ID NO: 520 - protein, 50 aa, Pseudoalteromonas sp.
PNLFTLPVAEITASEVERYSLYKLESAQDEDPFIATKFTDQMPISQLLRK

SEQ ID NO: 521 - protein, 50 aa, Pseudoalteromonas translucida
PNLFTLPVAEITASEVERYSLYKPESSQDEDPFIATKFTDRMPISQLLRK

SEQ ID NO: 522 - protein, 50 aa, Klebsiella oxytoca
ENPFYIPLENIKIYEVREYSGYEIDGAGKEDRLIPQQLTDRIPINQLLRK

SEQ ID NO: 523 - protein, 51 aa, Aeromonas salmonicida
VNPFLQPIDEIKACEVKQYSRYEIDAAGKEEVLNPLQFTDKIPISQLLKKR

SEQ ID NO: 524 - protein, 51 aa, Aeromonas salmonicida
ENPFLQSIDEIKACEVTQYSRYEINESGTEEVLNPLKFTDKIPISQLLKKR

SEQ ID NO: 525 - protein, 54 aa, Vibrio sp.
GENPFDEKVNMIQIQTIQQYTRFELDDKTGRRERFDRAFTALQQIPINKLLSKR

SEQ ID NO: 526 - protein, 49 aa, Aeromonas salmonicida
VTNPFTTELEKLLVPQVIEYEGFIIDPENGEIKFTKQIFKDIPLAALLG

SEQ ID NO: 527 - protein, 49 aa, Vibrio diazotrophicus
ISNPFEIELDKLLIPQIIEYEGYLLDSDSGDIKFTHQIFEDIPLTELLR

SEQ ID NO: 528 - protein, 49 aa, Vibrio fluvialis
ISNPFEIELDKLLISQIIEYEGYILDSDSGDIKFTHQIFEDIPLTELLR

SEQ ID NO: 529 - protein, 49 aa, Vibrio hyugaensis
DINPFTVQIDALTIEQIASYENYVTDAETGELRFVKQVFSKLSIQQLIG

SEQ ID NO: 530 - protein, 49 aa, Vibrio natriegens
DINPFTVQIDALTIEQIASYENYVTDAETGELRFVKQVFSKLSIQQLVG

SEQ ID NO: 531 - protein, 49 aa, Photobacterium aquae
EINPFSVDINELVIKQIESYEEYVPDAETGELKFVGQIFNALTIKQLLG

SEQ ID NO: 532 - protein, 49 aa, Vibrio azureus
EANPFSINADELVIKQIESYEEYVVDAANGELKFVQQIFNELTIEQLLG

SEQ ID NO: 533 - protein, 7 aa, synthetic construct
PKKKRKV

SEQ ID NO: 534 - protein, 30 aa, synthetic construct
EQKLISEEDLEQKLISEEDLEQKLISEEDL

SEQ ID NO: 535 - protein, 24 aa, synthetic construct
DYKDDDDKDYKDDDDKDYKDDDDK

SEQ ID NO: 536 - protein, 17 aa, synthetic construct
EGRGSLLTCGDVEENPG

SEQ ID NO: 537 - protein, 19 aa, synthetic construct
QCTNYALLKLAGDVESNPG

SEQ ID NO: 538 - protein, 18 aa, synthetic construct
ATNFSLLKQAGDVEENPG

SEQ ID NO: 539 - protein, 9 aa, synthetic construct
PAAKKKKLD

SEQ ID NO: 540 - protein, 16 aa, synthetic construct
KRPAATKKAGQAKKKK

SEQ ID NO: 541 - protein, 27 aa, synthetic construct
YPYDVPDYAYPYDVPDYAYPYDVPDYA

SEQ ID NO: 542 - protein, 14 aa, synthetic construct
GKPIPNPLLGLDST

SEQ ID NO: 543 - protein, 8 aa, synthetic construct
WSHPQFEK

SEQ ID NO: 544 - protein, 6 aa, synthetic construct
GSGGSG

SEQ ID NO: 545 - protein, 10 aa, synthetic construct
EQKLISEEDL

SEQ ID NO: 546 - protein, 8 aa, synthetic construct
DYKDDDDK

SEQ ID NO: 547 - protein, 14 aa, synthetic construct
KYAQQNSLFICSFP

SEQ ID NO: 548 - protein, 215 aa, Aeromonas hydrophila
MYRRHLKHSRVKNLFKFVSAKMNTVFTVESALEFDTCFHLEYSPSVKFYEAQPEGFYYEFAGRQCPYTPDFRLVDQNDSVSFLEIKPSDKVADPDFLHRFPLKQQRAIELSSPLKLVTEKQIRIDPILGNLKLLHRYSGFQSFTPLHMQLLGLVQKLGRVSLLRLSDSIDAPPEEVLASALSLIARGIMQSDLTVQKIGISSFVWAGGHSGIDHG

SEQ ID NO: 549 - protein, 624 aa, Aeromonas salmonicida; identical to SEQ ID NO: 2

SEQ ID NO: 550 - protein, 624 aa, Aeromonas salmonicida
MDKHNGGLFEDEFVIPQPSTSTSPIDAIQAVLPATVDSFPYVLKVEALHRRDYILWVEKNLAGGWTEKNLTPLLADAALVLPPPTPNWRTLARWRKIYIQHGRKLVSLIPKHQAKGNARSRLPPSDELFFEQAVHRYLVGEQPSIASAFQLYSDSIRIENLGVVENSIKTISYMAFYNRIKKLPAYQVMKSRKGSYIADVEFKAIASHKPPSRIMERVEIDHTPLDLLLLDDDLLVPLGRPSLTLLIDAYSHCVVGFNLNFNQPSYESVRNALLSSISKKDYVKNKYPSIEHEWPCYGKPETLVVDNGVEFWSASLAQSCLELGINIQYNPVRKPWLKPMIERMFGIINRKLLEPIPGKTFSNIQEKGDYDPQKDAVMRFSTFLEIFHHWVIDVYHYEPDSRYRYIPIISWQHGNKDAPPAPIIGDDLTKLEVILSLSLHCTHRRGGIQRYHLRYDSDELASYRMNYPDQTRGKRKVLVKLNPRDISYVYVFLEDLGSYIRVPCIDPIGYTKGLSLQEHQINVKLHRDFINEQMDVVSLSKARIYLNDRIKNELIEVRRNIRQRNVKGVNKIAKYRNVGSHAETSIVHELNHPATNEVISKMESASQPEHCDDWDNFTSGLEPY

SEQ ID NO: 551 - protein, 335 aa, Aeromonas salmonicida; identical to SEQ ID NO: 4

SEQ ID NO: 552 - protein, 335 aa, Aeromonas salmonicida
MDLSCHDADKLRSFIECYVETPLLRAIQEDFDRLRFNKQFAGEPQCMLLTGDTGTGKSSLIRHYAAKHPEQVRHGFIHKPLLVSRIPSRPTLESTMVELLKDLGQFGSSDRIHKSSAESLTEALIKCLKRCETELIIIDAFQELIENKTREKRNQIANRLKYISETAKIPIVLVGMPWATKIAEEPQWSSRLLIRRSIPYFKLSDDRENFIRLIMGLANRMPFETQARLETKHTIYALFAACYGSLRALKQLLDESVKQALAAHAETLKHEHIAVAYALFYPDQVNPFLQPIDEIKACEVKQYSRYEIDAAGKEEVLNPLQFTDKIPISQLLKKR

SEQ ID NO: 553 - protein, 335 aa, Aeromonas salmonicida
MDLSCHDADKLRSFIECYVETPLLRAIQEDFDRLRFNKQFAGEPQCMLLTGDTGTGKSSLIRHYAAKHPEQVRHGFIHKPLLVSRIPSRPTLESTMVELLKDLGQFGSSDRIHKSSAESLTEALIKCLKRCETELIIIDQFQELIENKTREKRNQIANRLKYISETAKIPIVLVGMPWATKIAEEPQWSSRLLIRRSIPYFKLSDDRENFIRLIMGLANRMPFETQARLETKHTIYALFAACYGSLRALKQLLDESVKQALAAHAETLKHEHIAVAYALFYPDQVNPFLQPIDEIKACEVKQYSRYEIDAAGKEEVLNPLQFTDKIPISQLLKKR

SEQ ID NO: 554 - protein, 216 aa, synthetic construct; identical to SEQ ID NO: 258

SEQ ID NO: 555 - protein, 357 aa, synthetic construct; identical to SEQ ID NO: 256

SEQ ID NO: 556 - protein, 714 aa, synthetic construct
MPKKKRKVGSGVTIMHIEELLDIEDHGERDRQLRRYLAPYSAEIGVDGAEKMALVVLLNLTLKRDRVESLCDEGLARQLLSDEGHITNCLHTVRWLHTHNLKYPDARVSGERLIINAPPLIPGVISSAGLPMRMGWAHDSSDINLAKLFGTSFRYRDDSTNLALQLVARSKTWEQALIGLGLTQQQLDIWCQLLASNLENNTFPTVVSPFSKQVRFLYQGNYCVVTPVVSHALLAQLQNVVHEKKLQCTYIHHDHPASVGSLVGALGGKVAVLDYPPPVSPDKARSFSQARKHRLANGQSLFDRSVFNDHVFIDALKHVISRPGLTRKQQRQLRLSALRYLRRQLAIWLGPIIEWRDEIVSSGRGEPGNLPSGGLELELITQPKKMLPELMLQVAGRFHLELQNHSAGRRFAFHPALMAPIKSQILWLLRQLADDEEKDEPHPPTSCYYLHLSGLTVYDASALANPYLCGIPSLSALAGFCHDYERRLQSLIGQSVYFRGLAWYLGRYSLVTGKHLPEPSKSADPKSVSAIRRPGLLDGRYCDLGMDLIIEVHIPTGGSLPFTTCLDLLRVALPARFAGGCLHPPSLYEEYNWCTVYQDKSTLFTVLSRLPRYGCWIYPSDADLRSFEELSEALALDRRLRPVATGFVFLEEPVERAGSIEGQHVYAESAIGTALCINPVEMRLAGKKRFFGAGFWQLNDAKGAILMNGSANTG

SEQ ID NO: 557 - protein, 882 aa, synthetic construct; identical to SEQ ID NO: 3

SEQ ID NO: 558 - protein, 345 aa, synthetic construct
MDLSCHDADKLRSFIECYVETPLLRAIQEDFDRLRFNKQFAGEPQCMLLTGDTGTGKSSLIRHYAAKHPEQVRHGFIHKPLLVSRIPSRPTLESTMVELLKDLGQFGSSDRIHKSSAESLTEALIKCLKRCETELIIIDEFQELIENKTREKRNQIANRLKYISETAKIPIVLVGMPWATKIAEEPQWSSRLLIRRSIPYFKLSDDRENFIRLIMGLANRMPFETQARLETKHTIYALFAACYGSLRALKQLLDESVKQALAAHAETLKHEHIAVAYALFYPDQVNPFLQPIDEIKACEVKQYSRYEIDAAGKEEVLNPLQFTDKIPISQLLKKRGSGPKKKRKV

SEQ ID NO: 559 - protein, 453 aa, synthetic construct; identical to SEQ ID NO: 7

(19) United States
(12) Patent Application Publication
(10) Pub. No.: US 2025/0197458 A1
(43) Pub. Date: Jun. 19, 2025

(54) ADAPTATIONS FOR HIGH EFFICIENCY I-F3-CRISPR-CAS SYSTEMS FOR GUIDE RNA-DIRECTED TRANSPOSITION IN HUMAN CELLS

(71) Applicants: Cornell University, Ithaca, NY (US); Graphite Bio, Inc., South San Francisco, CA (US)

(72) Inventors: Joseph E. PETERS, Ithaca, NY (US); Robert WINGO, Ithaca, NY (US); Michael PETASSI, Brooklyn, NY (US); Daniel DEVER, South San Francisco, CA (US); Beeke WIENERT, South San Francisco, CA (US)

(21) Appl. No.: 18/835,977

(22) PCT Filed: Feb. 9, 2023
(86) PCT No.: PCT/US2023/062327

§ 371 Date: Aug. 5, 2024

Related U.S. Application Data
(60) Provisional application No. 63/308,451, filed on Feb. 9, 2022.

(51) Int. Cl. C07K 14/195; C12N 9/22; C12N 15/11; C12N 15/90

(52) U.S. Cl. CPC C07K 14/195; C12N 9/22; C12N 15/11; C12N 15/902; C12N 2310/20

(57) ABSTRACT

Provided are compositions and methods for modifying DNA substrates. The compositions include modified I-F3 proteins for use in a CRISPR systems to modify a DNA substrate. The modified proteins include I-F3 TnsC, TniQ, TnsA, TnsB and fusion proteins containing TnsA and TnsB, Cas8, Cas5, Cas7, and Cas6 modified proteins. The CRISPR systems include a guide RNA. Protein modifications provide for a higher transposition frequency than unmodified I-F3 CRISPR systems.

### CROSS-REFERENCE TO RELATED APPLICATION

This application claims priority to U.S. provisional application No. 63/308,451, filed Feb. 9, 2022, the entire disclosure of which is incorporated herein by reference.

### FIELD

The present disclosure relates generally to approaches for modifying DNA, and more particularly, to improved compositions and methods for CRISPR-based editing that involve modified proteins.

### SEQUENCE LISTING

The instant application contains a sequence listing which has been submitted in .xml format and is hereby incorporated by reference in its entirety. Said .xml file is named “018617_01398_ST26.xml”, was created on Feb. 9, 2023, and is 697,220 bytes in size.

### BACKGROUND

Despite the brisk activity with engineering new Clustered Regularly Interspaced Short Palindromic Repeats (CRISPR)-Cas genome modification tools, unmet challenges remain. This is particularly true where insertion of large DNA cargos is desired. Many available strategies for integrating DNA cargo involve making a DNA double strand break with a CRISPR-Cas system and provoking the host to carry out repair using the DNA cargo with sufficient flanking homology to allow integration of the genetic information. This is an inefficient process that can also introduce unwanted ancillary mutations and additional damaging effects from inducing the host DNA damage response. There is an ongoing need for improved methods of using CRISPR systems to introduce DNA cargos into selected locations. The present disclosure is pertinent to this need.

### BRIEF SUMMARY

The present disclosure provides improved compositions and methods for modifying DNA substrates, such as chromosomes, plasmids and organelle DNA. The composition include modified I-F3 proteins for use in CRISPR systems to modify a DNA substrate. The modified proteins include TnsC proteins comprising an insertion or substitution of one or more amino acids; TnsA proteins comprising an insertion or substitution of one or more amino acids; TnsB protein comprising an insertion or substitution of one or more amino acids; and a single protein comprising the amino acid sequence of a TnsA protein and the amino acid sequence of a TnsB protein. The single protein may comprise a modified TnsA segment, a modified TnsB segment, and/or an insertion of one or more amino acids between the TnsA and TnsB segments. Modified Cas8, Cas5, Cas7, and Cas6 proteins are also provided. In embodiments, CRISPR systems that include a guide RNA and one or more modified proteins exhibit a higher transposition frequency relative to an I-F3 system comprising the same guide RNA and I-F3 proteins in unmodified form. The described compositions and methods may be used to insert a DNA template into a target chromosome or plasmid in a guide RNA-directed manner.

Polynucleotides encoding one or more of the described proteins, and methods of using the polynucleotides and the proteins for modifying prokaryotic and eukaryotic cells are also provided. Cells modified to comprise the modified proteins and polynucleotides are also provided.

### BRIEF DESCRIPTION OF THE FIGURES

FIG. 1—Analysis of protein tags for the effect on Guide RNA directed transposition with the Tn6900 element from Aeromonas salmonicida S44—Transposition was monitored by the mate-out assay. In the assay the target is on a mobilizable plasmid and a mini element (mini element=the left and right transposon ends flanking an antibiotic resistance gene for use as a genetic marker) resides at a neutral position in the chromosome. Transposition was monitored after inducing transposition with the TnsA, TnsB, TnsC, TniQ, Cas8-5, Cas7, and Cas6 proteins and guide RNA with atypical repeats flanking a spacer matching a site in lacZ (lacZ4). In panel A, TnsA, TnsB, TnsC are encoded as an operon on an expression plasmid (pACYClac), Cas8-5, Cas7, and Cas6 are encoded as an operon on an expression plasmid (pBAD322), TniQ or tagged derivatives are encoded on an expression plasmid (pBBRara), and guide RNA is encoded on an expression plasmid (pCDFara). In panel B, TnsA, TnsB, TnsC are encoded as an operon on an expression plasmid (pACYClac), TniQ, Cas7, and Cas6 are encoded as an operon on an expression plasmid (pBAD322), Cas8-5 or tagged derivatives are encoded on an expression plasmid (pBBRara), and guide RNA is encoded on an expression plasmid (pCDFara). A genetic marker on the mobile plasmid was used to mate the plasmid into a new host where a genetic marker on the transposon is used to determine the percent of the mobile plasmids that were targeted for transposition. Each example was tested three times with the mean+standard deviation graphed. For tagged derivatives, percent activity is also shown with respect to the untagged protein (TniQ or Cas8-5). Tags that were tested are an SV40 Nuclear Localization Sequence (NLS)=PKKKRKV (SEQ ID NO:533), 3×Myc=EQKLISEEDLEQKLISEEDLEQKLISEEDL (SEQ ID NO:534), 3×FLAG=DYKDDDDKDYKDDDDKDYKDDDDK (SEQ ID NO:535), T2A=EGRGSLLTCGDVEENPG (SEQ ID NO:536), E2A=QCTNYALLKLAGDVESNPG (SEQ ID NO: 537), and P=single proline. All tags are separated by a GSG linker indicated by thick black line.

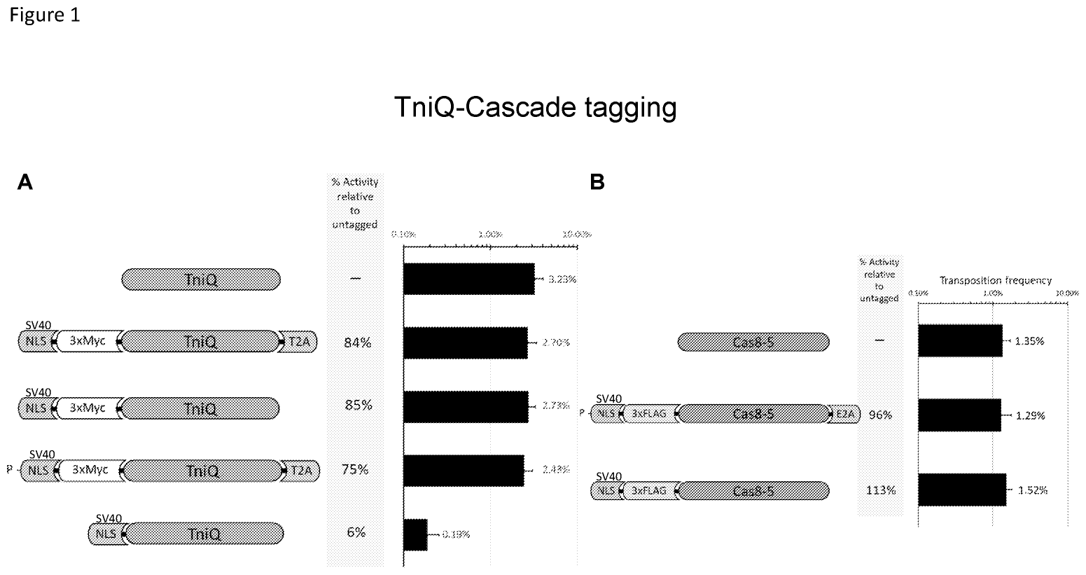

FIG. 2—Analysis of protein tags for the effect on Guide RNA directed transposition with the Tn6900 element from Aeromonas salmonicida S44—Transposition was monitored by the mate-out assay. In the assay the target is on a mobilizable plasmid and a mini element (mini element=the left and right transposon ends flanking an antibiotic resistance gene for use as a genetic marker) resides at a neutral position in the chromosome. Transposition was monitored after inducing transposition with the TnsA, TnsB, TnsC, TniQ, Cas8-5, Cas7, and Cas6 proteins and guide RNA with atypical repeats flanking a spacer matching a site in lacZ (lacZ4). In panel A, TnsA, TnsB, TnsC are encoded as an operon on an expression plasmid (pACYClac), TniQ, Cas8-5, and Cas6 are encoded as an operon on an expression plasmid (pBAD322), Cas7 or tagged derivatives are encoded on an expression plasmid (pBBRara), and guide RNA is encoded on an expression plasmid (pCDFara). In panel B, TnsA, TnsB, TnsC are encoded as an operon on an expression plasmid (pACYClac), TniQ, Cas8-5, and Cas7 are encoded as an operon on an expression plasmid (pBAD322), Cas6 or tagged derivatives are encoded on an expression plasmid (pBBRara), and guide RNA is encoded on an expression plasmid (pCDFara). A genetic marker on the mobile plasmid was used to mate the plasmid into a new host where a genetic marker on the transposon is used to determine the percent of the mobile plasmids that were targeted for transposition. Each example was tested three times with the mean+standard deviation graphed. For tagged derivatives, percent activity is also shown with respect to the untagged protein (Cas7 or Cas6). Tags that were tested are an SV40 Nuclear Localization Sequence (NLS)=PKKKRKV (SEQ ID NO:533), T2A=EGRGSLLTCGDVEENPG (SEQ ID NO: 536), and P2A=ATNFSLLKQAGDVEENPG (SEQ ID NO:538). All tags are separated by a GSG linker indicated by thick black line. Inset shows changes in the overall transposition frequency as a function of vectors used in the assay, with Cas6 either encoded in standard operon form or on a separate plasmid as in the main graph.

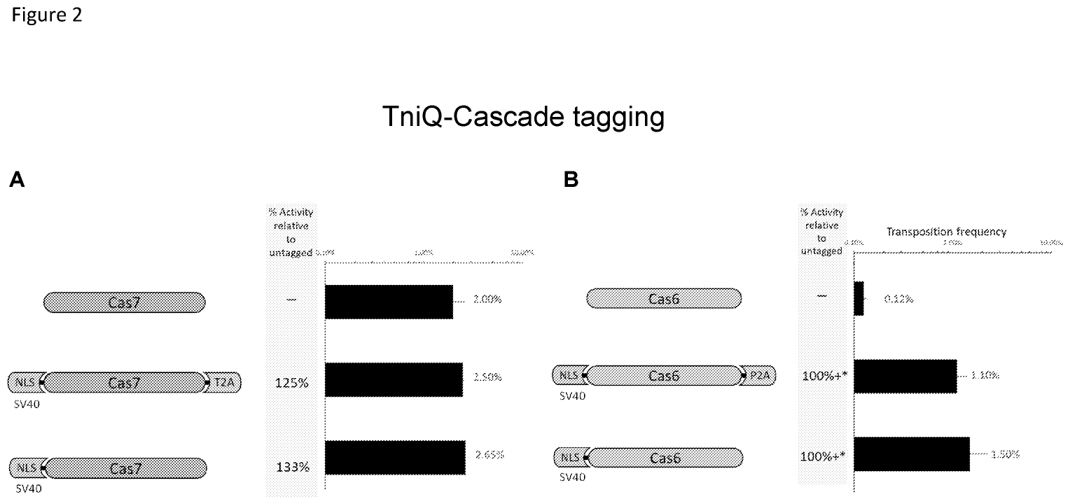

FIG. 3—Analysis of TnsA and TnsB fusions with different protein tags for the effect on guide RNA directed transposition with the Tn6900 element from Aeromonas salmonicida S44—Transposition was monitored by the mate-out assay. In the assay the target is on a mobilizable plasmid and a mini element (mini element=the left and right transposon ends flanking an antibiotic resistance gene for use as a genetic marker) resides at a neutral position in the chromosome. Transposition was monitored after inducing transposition with the TnsAB fusion, TnsC, TniQ, Cas8-5, Cas7, and Cas6 proteins and guide RNA with atypical repeats flanking a spacer matching a site in lacZ (lacZ4). TnsAB fusion, generated by insertion of two bp between coding regions to shift to a continuous reading frame including both proteins, or tagged derivatives are encoded on an expression plasmid (pBBRlac), TnsC is encoded on an expression plasmid (pACYClac), TniQ, Cas8-5, Cas7 and Cas6 are encoded as an operon on an expression plasmid (pBAD322), and guide RNA is encoded on an expression plasmid (pCDFara). A genetic marker on the mobile plasmid was used to mate the plasmid into a new host where a genetic marker on the transposon is used to determine the percent of the mobile plasmids that were targeted for transposition. Each example was tested three times with the mean+standard deviation graphed. Tags that were tested are an SV40 Nuclear Localization Sequence (SV40)=PKKKRKV (SEQ ID NO:533), alternate NLS (NLS^{alt})=PAAKKKKLD (SEQ ID NO:539), Nucleoplasmin NLS (Nucleoplasmin)=KRPAATKKAGQAKKKK (SEQ ID NO:540), and 3×HA=YPYDVPDYAYPYDVPDYAYPYDVPDYA (SEQ ID NO:541). All tags are separated by a GSG linker indicated by thick black line.

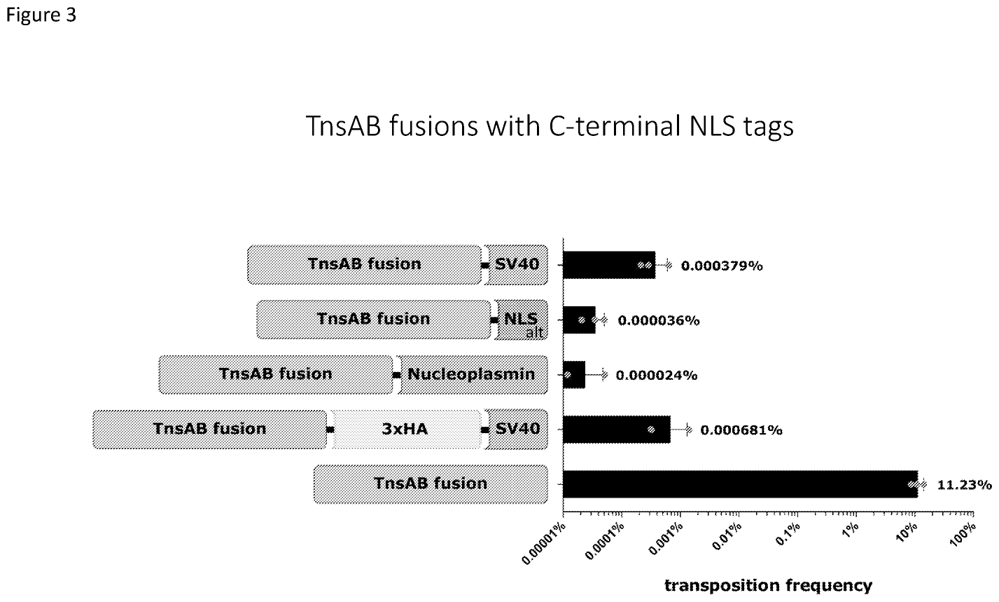

FIG. 4—Analysis of TnsA and TnsB fusion proteins with different protein tags for the effect on Guide RNA directed transposition with the Tn6900 element from Aeromonas salmonicida S44—Transposition was monitored by the mate-out assay. In the assay the target is on a mobilizable plasmid and a mini element (mini element=the left and right transposon ends flanking an antibiotic resistance gene for use as a genetic marker) resides at a neutral position in the chromosome. Transposition was monitored after inducing transposition with the TnsAB fusion, TnsC, TniQ, Cas8-5, Cas7, and Cas6 proteins and guide RNA with atypical repeats flanking a spacer matching a site in lacZ (lacZ4). TnsAB fusion, generated by insertion of two bp between coding regions to shift to a continuous reading frame including both proteins, or tagged derivatives with tags inserted between the proteins as indicated, are encoded on an expression plasmid (pBBRlac), TnsC is encoded on an expression plasmid (pACYClac), TniQ, Cas8-5, Cas7 and Cas6 are encoded as an operon on an expression plasmid (pBAD322), and guide RNA is encoded on an expression plasmid (pCDFara). A genetic marker on the mobile plasmid was used to mate the plasmid into a new host where a genetic marker on the transposon is used to determine the percent of the mobile plasmids that were targeted for transposition. Each example was tested three times with the mean+standard deviation graphed. Tags that were tested are an SV40 Nuclear Localization Sequence (SV40)=PKKKRKV (SEQ ID NO:533), alternate NLS (NLS^{alt})=PAAKKKKLD (SEQ ID NO:539), Nucleoplasmin NLS (Nucleoplasmin)=KRPAATKKAGQAKKKK (SEQ ID NO:540), and 3×HA=YPYDVPDYAYPYDVPDYAYPYDVPDYA (SEQ ID NO: 541). All tags are separated by a GSG linker indicated by thick black line.

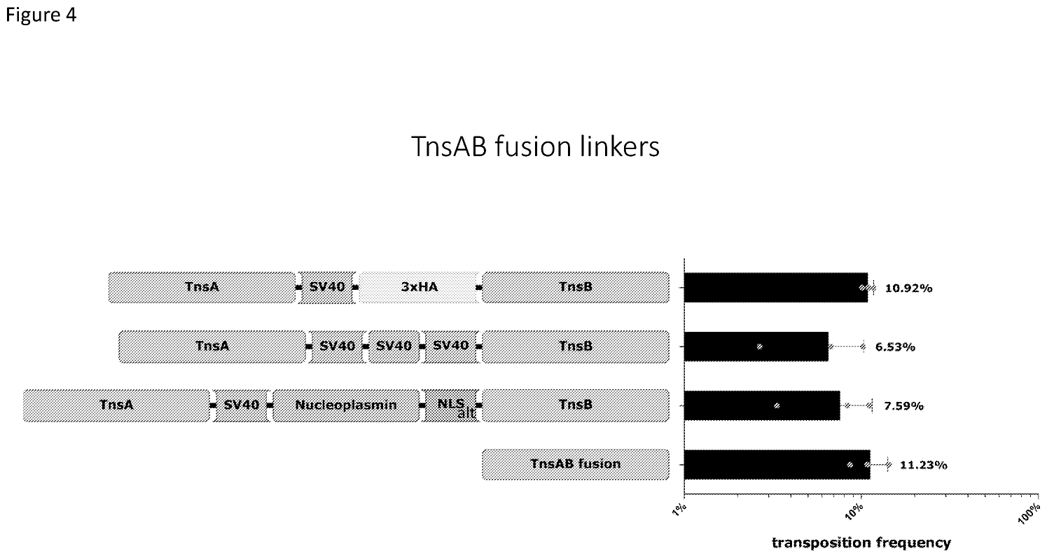

FIG. 5—Analysis of protein tags on TnsC for the effect on Guide RNA directed transposition with the Tn6900 element from Aeromonas salmonicida S44—Transposition was monitored by the mate-out assay. In the assay the target is on a mobilizable plasmid and a mini element (mini element=the left and right transposon ends flanking an antibiotic resistance gene for use as a genetic marker) resides at a neutral position in the chromosome. Transposition was monitored after inducing transposition with the TnsA, TnsB, TnsC, TniQ, Cas8-5, Cas7, and Cas6 proteins and guide RNA with atypical repeats flanking a spacer matching a site in lacZ (lacZ4). TnsA and TnsB are encoded on an expression plasmid (pBBRlac), TnsC or tagged derivative is encoded on an expression plasmid (pACYClac), TniQ, Cas8-5, Cas7 and Cas6 are encoded as an operon on an expression plasmid (pBAD322), and guide RNA is encoded on an expression plasmid (pCDFara). A genetic marker on the mobile plasmid was used to mate the plasmid into a new host where a genetic marker on the transposon is used to determine the percent of the mobile plasmids that were targeted for transposition. Each example was tested three times with the mean+standard deviation graphed. Tags that were tested are 3×FLAG=DYKDDDDKDYKDDDDKDYKDDDDK (SEQ ID NO:535), SV40 Nuclear Localization Sequence (SV40)=PKKKRKV (SEQ ID NO:533), alternate NLS (NLS^{alt})=PAAKKKKLD (SEQ ID NO:539), and Nucleoplasmin NLS (NP NLS)=KRPAATKKAGQAKKKK (SEQ ID NO: 540). All tags are separated by a GSG linker indicated by thick black line.

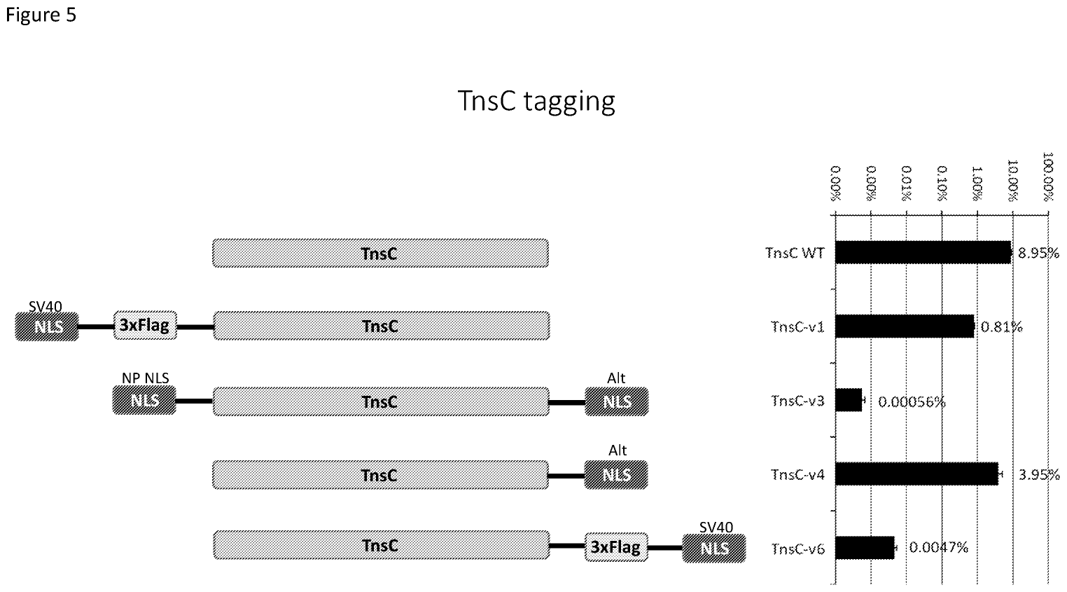

FIG. 6—Analysis of protein tags on TnsC for the effect on Guide RNA directed transposition with the Tn6900 element from Aeromonas salmonicida S44—Transposition was monitored by the mate-out assay. In the assay the target is on a mobilizable plasmid and a mini element (mini element=the left and right transposon ends flanking an antibiotic resistance gene for use as a genetic marker) resides at a neutral position in the chromosome. Transposition was monitored after inducing transposition with the TnsA, TnsB, TnsC, TniQ, Cas8-5, Cas7, and Cas6 proteins and guide RNA with atypical repeats flanking a spacer matching a site in lacZ (lacZ4). TnsA and TnsB are encoded on an expression plasmid (pBBRlac), TnsC or tagged derivative is encoded on an expression plasmid (pACYClac), TniQ, Cas8-5, Cas7 and Cas6 are encoded as an operon on an expression plasmid (pBAD322), and guide RNA is encoded on an expression plasmid (pCDFara). A genetic marker on the mobile plasmid was used to mate the plasmid into a new host where a genetic marker on the transposon is used to determine the percent of the mobile plasmids that were targeted for transposition. Each example was tested three times with the mean+standard deviation graphed. Tags that were tested are Strep=WSHPQFEK (SEQ ID NO:543), SV40 Nuclear Localization Sequence (SV40)=PKKKRKV (SEQ ID NO:533), alternate NLS (NLS^{alt})=PAAKKKKLD (SEQ ID NO:539), and Nucleoplasmin NLS (NP NLS)=KRPAATKKAGQAKKKK (SEQ ID NO:540). All tags are separated by a GSG linker indicated by thick black line.

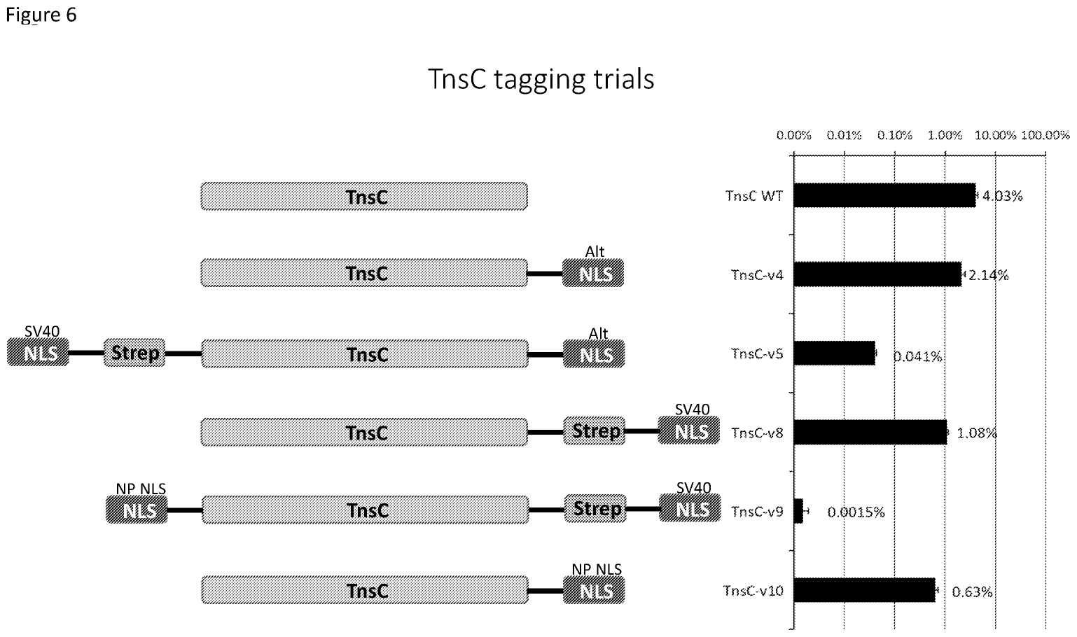

FIG. 7—Analysis of protein tags on TnsC for the effect on Guide RNA directed transposition with the Tn6900 element from Aeromonas salmonicida S44—Transposition was monitored by the mate-out assay. In the assay the target is on a mobilizable plasmid and a mini element (mini element=the left and right transposon ends flanking an antibiotic resistance gene for use as a genetic marker) resides at a neutral position in the chromosome. Transposition was monitored after inducing transposition with the TnsA, TnsB, TnsC, TniQ, Cas8-5, Cas7, and Cas6 proteins and guide RNA with atypical repeats flanking a spacer matching a site in lacZ (lacZ4). TnsA and TnsB are encoded on an expression plasmid (pBBRlac), TnsC or tagged derivative is encoded on an expression plasmid (pACYClac), TniQ, Cas8-5, Cas7 and Cas6 are encoded as an operon on an expression plasmid (pBAD322), and guide RNA is encoded on an expression plasmid (pCDFara). A genetic marker on the mobile plasmid was used to mate the plasmid into a new host where a genetic marker on the transposon is used to determine the percent of the mobile plasmids that were targeted for transposition. Each example was tested three times with the mean+standard deviation graphed. Tags that were tested are V5=GKPIPNPLLGLDST (SEQ ID NO:542), Strep=WSHPQFEK (SEQ ID NO:543), SV40 Nuclear Localization Sequence (SV40)=PKKKRKV (SEQ ID NO:533), alternate NLS (NLS^{alt})=PAAKKKKLD (SEQ ID NO:539), E2A=QCTNYALLKLAGDVESNPG (SEQ ID NO:537), P2A=ATNFSLLKQAGDVEENPG (SEQ ID NO:538), and P=single proline. All tags are separated by a GSG linker indicated by thick black line. Two separated black lines indicate two GSG linkers (GSGGSG) (SEQ ID NO:544).

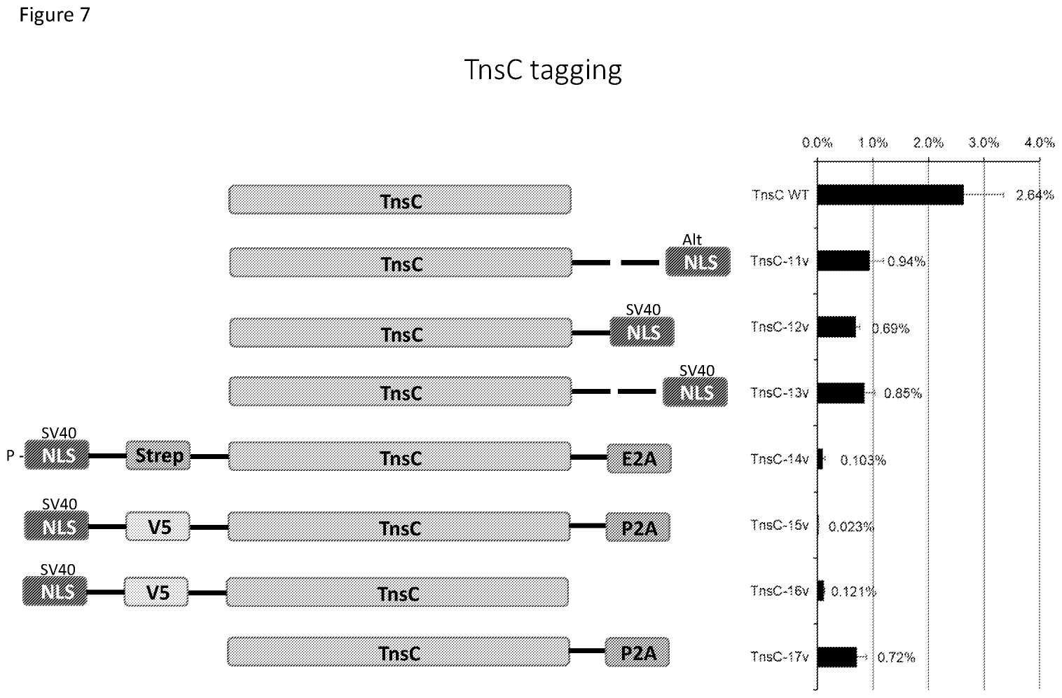

FIG. 8—Analysis of protein tags on TnsC for the effect on Guide RNA directed transposition with the Tn6900 element from Aeromonas salmonicida S44—Transposition was monitored by the mate-out assay. In the assay the target is on a mobilizable plasmid and a mini element (mini element=the left and right transposon ends flanking an antibiotic resistance gene for use as a genetic marker) resides at a neutral position in the chromosome. Transposition was monitored after inducing transposition with the TnsA, TnsB, TnsC, TniQ, Cas8-5, Cas7, and Cas6 proteins and guide RNA with atypical repeats flanking a spacer matching a site in lacZ (lacZ4). TnsA and TnsB are encoded on an expression plasmid (pBBRlac), TnsC or tagged derivative is encoded on an expression plasmid (pACYClac), TniQ, Cas8-5, Cas7 and Cas6 are encoded as an operon on an expression plasmid (pBAD322), and guide RNA is encoded on an expression plasmid (pCDFara). A genetic marker on the mobile plasmid was used to mate the plasmid into a new host where a genetic marker on the transposon is used to determine the percent of the mobile plasmids that were targeted for transposition. Each example was tested three times with the mean+standard deviation graphed. Tags that were tested are SV40 Nuclear Localization Sequence (SV40)=PKKKRKV (SEQ ID NO:533), 3×Myc=EQKLISEEDLEQKLISEEDLEQKLISEEDL (SEQ ID NO: 534), 1×Myc=EQKLISEEDL (SEQ ID NO:545). All tags are separated by a GSG linker indicated by thick black line.

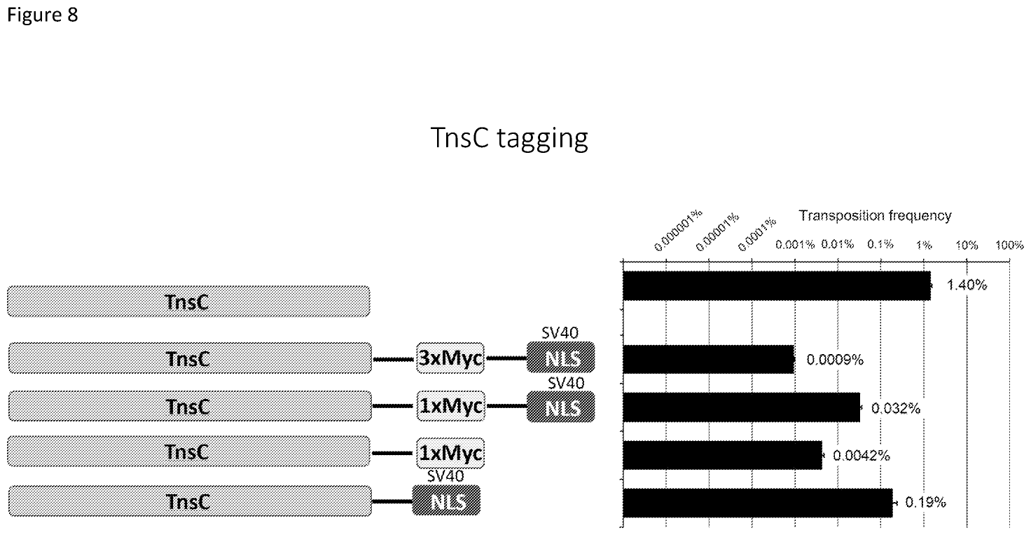

FIG. 9—Analysis of internal positions for the FLAG tag on TnsC for the effect on Guide RNA directed transposition with the Tn6900 element from Aeromonas salmonicida S44—Transposition was monitored by the mate-out assay. In the assay the target is on a mobilizable plasmid and a mini element (mini element=the left and right transposon ends flanking an antibiotic resistance gene for use as a genetic marker) resides at a neutral position in the chromosome. Transposition was monitored after inducing transposition with the TnsA, TnsB, and TnsC proteins, guide RNA with atypical repeats flanking a spacer matching a site in lacZ (lacZ4), testing with and without TniQ-Cascade (Cas8-5, Cas7, and Cas6) proteins. TnsA and TnsB are encoded on an expression plasmid (pBBRlac), TnsC or tagged derivative with FLAG=DYKDDDDK (SEQ ID NO:546) inserted at the indicated amino acid position is encoded on an expression plasmid (pACYClac), TniQ, Cas8-5, Cas7 and Cas6 are encoded as an operon on an expression plasmid (pBAD322), and guide RNA is encoded on an expression plasmid (pCDFara). A genetic marker on the mobile plasmid was used to mate the plasmid into a new host where a genetic marker on the transposon is used to determine the percent of the mobile plasmids that were targeted for transposition. For the S304-FLAG the ability to target the lacZ gene was also monitored, indicated as a percentage on-target (i.e., inactivating lacZ gene by insertion, assessed by X-gal indicator media) versus off-target (i.e., not inactivating the lacZ gene). Each example was tested three times with the mean+standard deviation graphed.

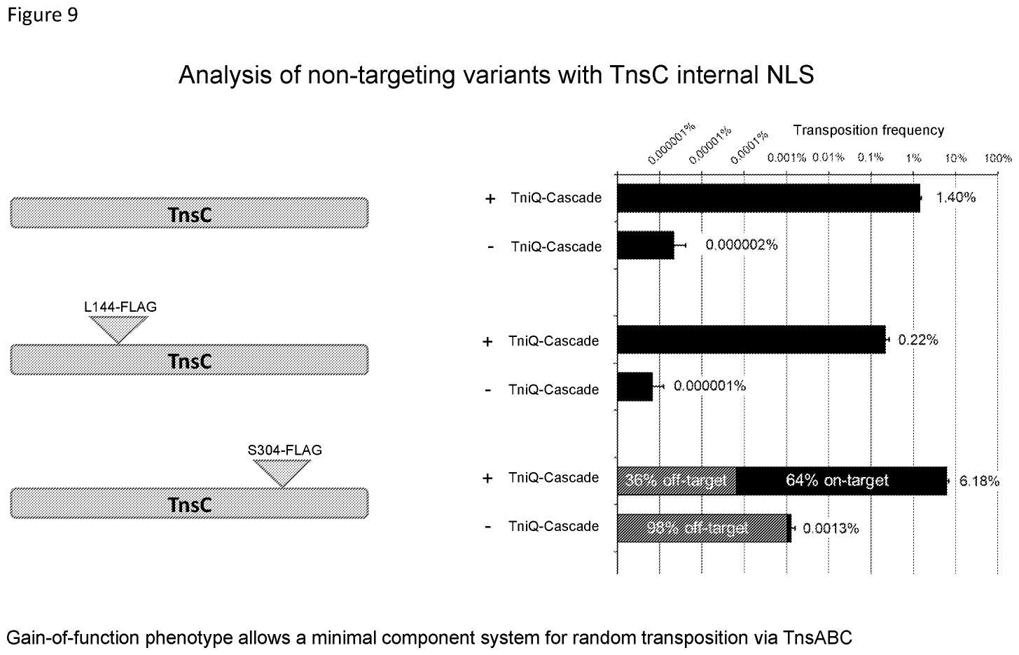

FIG. 10—Analysis at two internal positions for the effect of the NLS or tag within TnsC on Guide RNA directed transposition with the Tn6900 element from Aeromonas salmonicida S44—Transposition was monitored by the mate-out assay. In the assay the target is on a mobilizable plasmid and a mini element (mini element=the left and right transposon ends flanking an antibiotic resistance gene for use as a genetic marker) resides at a neutral position in the chromosome. Transposition is monitored after inducing transposition with the TnsA, TnsB, and TnsC derivatives, guide RNA with atypical repeats flanking a spacer matching a site in lacZ (lacZ4), testing with untagged TniQ-Cascade (Cas8-5, Cas7, and Cas6) proteins. TnsA and TnsB are encoded on an expression plasmid (pBBRlac), TnsC wild type (wt) or with tagged derivatives (Alt, SV40, NP or 3×FLAG). SV40 Nuclear Localization Sequence (SV40)=PKKKRKV (SEQ ID NO:533), Alt NLS (Alt)=PAAKKKKLD (SEQ ID NO:539), 3×FLAG=DYKDDDDKDYKDDDDKDYKDDDDK (SEQ ID NO:535) or Nucleoplasmin NLS (NP)=KRPAATKKAGQAKKKK (SEQ ID NO:540) inserted at the indicated amino acid position is encoded on an expression plasmid (pACYClac), TniQ, Cas8-5, Cas7 and Cas6 are encoded as an operon on an expression plasmid (pBAD322), and guide RNA is encoded on an expression plasmid (pCDFara). A genetic marker on the mobile plasmid is used to mate the plasmid into a new host where a genetic marker on the transposon is used to determine the percent of the mobile plasmids that were targeted for transposition. Each example was tested three times with the mean+standard deviation graphed.

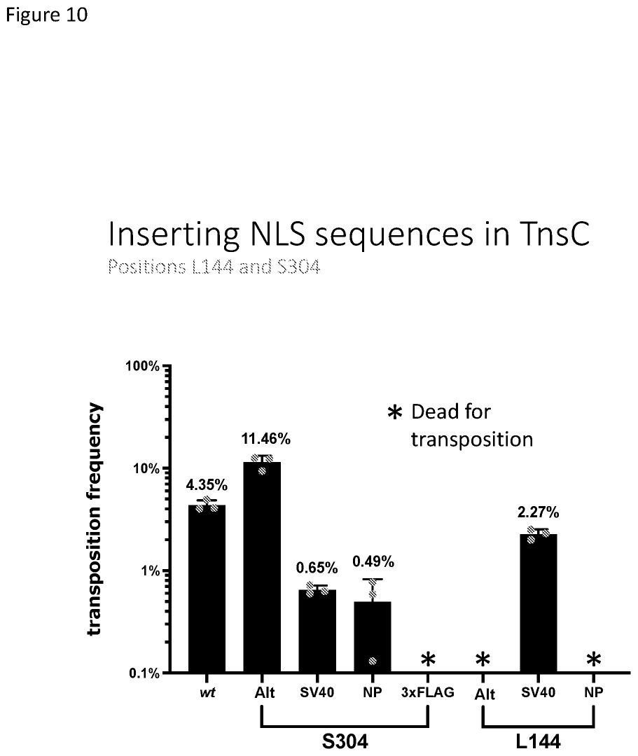

FIG. 11—Analysis of the effect of combining fusions and tags on Guide RNA directed transposition with the Tn6900 element from Aeromonas salmonicida S44—Transposition was monitored by the mate-out assay. In the assay the lacZ target is on a mobilizable plasmid and a mini element (mini element=the left and right transposon ends flanking an antibiotic resistance gene for use as a genetic marker) resides at a neutral position in the chromosome. Transposition is monitored after inducing transposition with the TnsA, TnsB, TnsC, TniQ, Cas8-5, Cas7, and Cas6 proteins and guide RNA with atypical repeats. TnsA and TnsB or tagged fusion protein are encoded on an expression plasmid (pBBRlac), TnsC or tagged derivative is encoded on an expression plasmid (pACYClac), TniQ, Cas8-5, Cas7 and Cas6 (Q-Cascade) are encoded on an expression plasmid (pBAD322), and guide RNA is encoded on an expression plasmid (pCDFara). Guide—Either a lacZ specific guide was tested (lacZ4) or a nontargeting guide (nt) as a control. TnsC—TnsC was either wild-type and untagged (No) or with a C-terminal alternate NLS tag (TnsC-Alt NLS, as in FIG. 6). TnsAB—TnsA and TnsB were either in their wild-type and unfused form (No) or fused with an intervening NLS and 3×HA tag (Tag). Q-Cascade—TniQ, Cas8-5, Cas7, and Cas6 (Q-Cascade) was either in the native operon form as found in the original A. salmonicida host (Native operon), a synthetic operon with reading frames separated by optimized ribosome loading site sequences with wild-type untagged proteins (Synthetic No Tags), or a synthetic operon with reading frames separated by optimized ribosome loading site sequences with tagged proteins (Synthetic Tagged). Synthetic Tagged alleles are as follows—TniQ=SV40NLS-3×Myc-TniQ as in FIG. 1, Cas8-5=SV40NLS-Cas8-5 as in FIG. 1, Cas7=SV40NLS-Cas7 as in FIG. 2, and Cas6=SV40NLS-Cas6 as in FIG. 2. A genetic marker on the mobile plasmid is used to mate the plasmid into a new host where a genetic marker on the transposon is used to determine the percent of the mobile plasmids that were targeted for transposition. Each example was tested three times with the mean+standard deviation graphed.

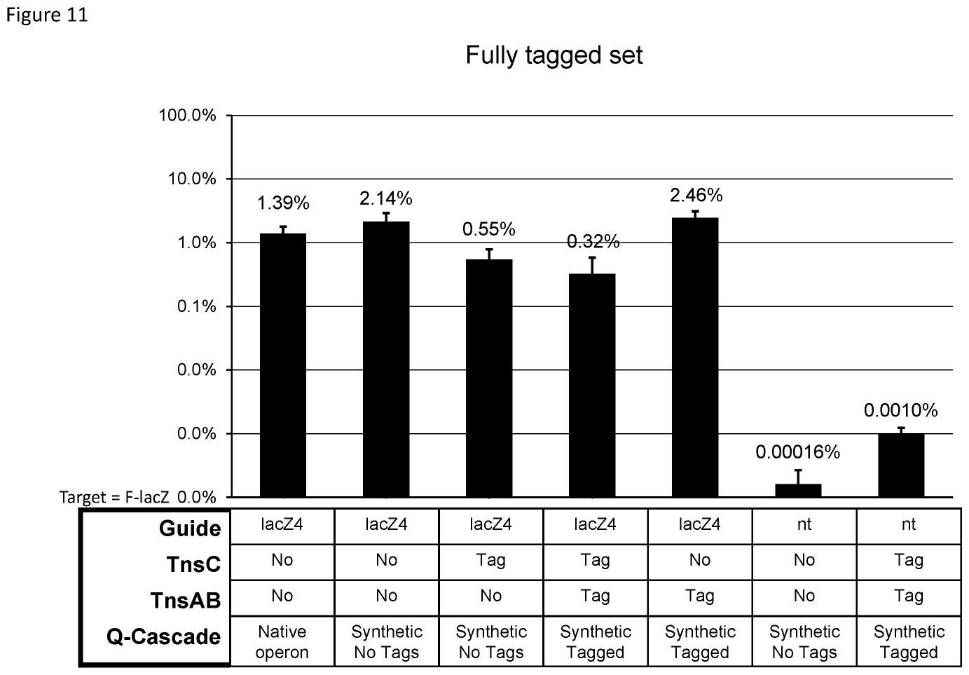

FIG. 12—Representative type I-F3 CRISPR-Cas transposons analyzed with the TnsAB and TnsC fusion strategy. Each element is listed with an internal tracing number 0-42 and either the strain identifier or Tn ####number. Transposon Tn6022 is not a type I-F3 CRISPR-Cas transposon but is from a sister group that was included as an outgroup to make the similarity tree. The similarity tree was constructed with FastTree using the sequence alignments of TnsA, TnsB, TnsC proteins from all elements made with MUSCLE.

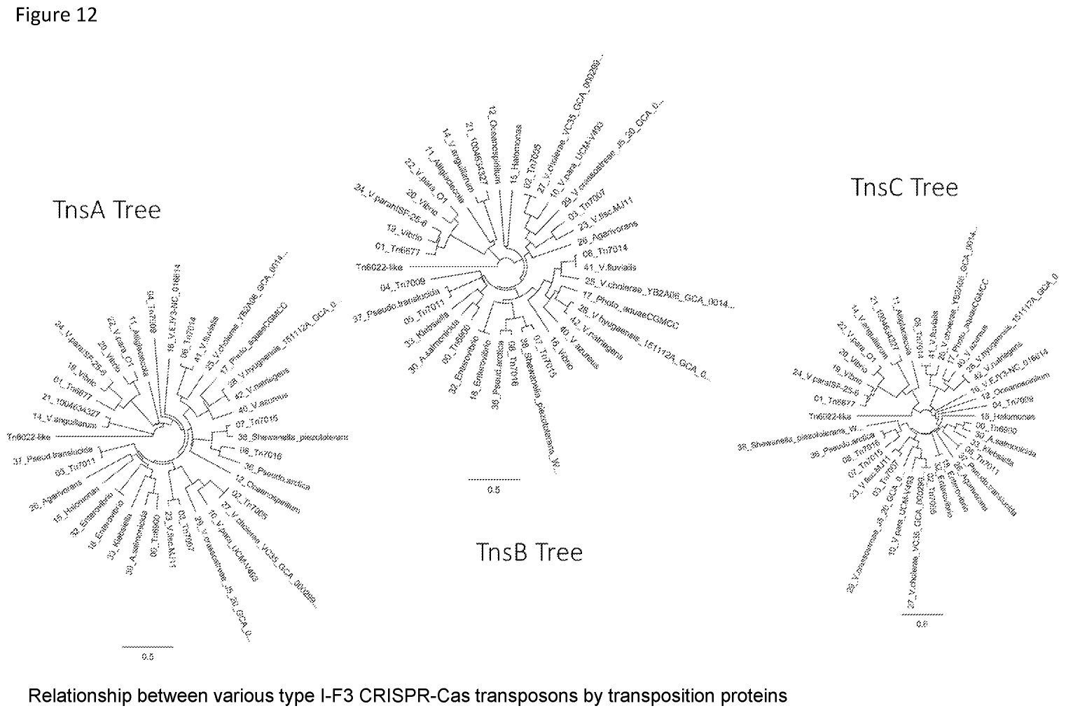

FIG. 13—All of the type I-F3 CRISPR-Cas transposons that were tested with the fusing and tagging strategy to allow minimal transposition with TnsA, TnsB and TnsC—TnsA and TnsB were fused with an intervening NLS and 3×HA tag and NLS was included at the internal S304 position (or equivalent). A previous transposon number (Tn ####) it is included, all are listed by the strain of origin.

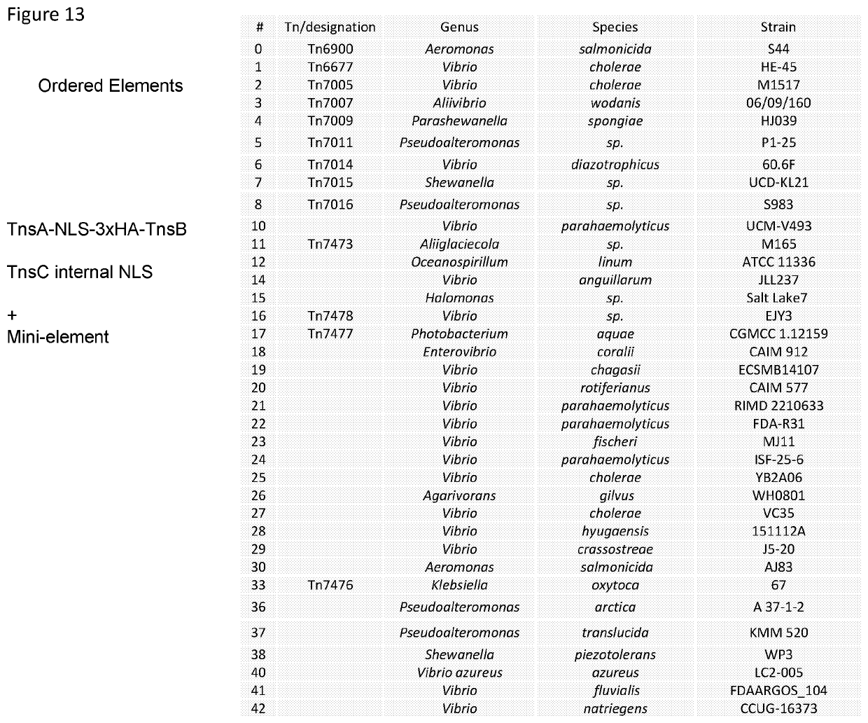

FIG. 14—Type I-F3 CRISPR-Cas transposons that were tested with the fusing and tagging strategy to allow transposition with TnsA, TnsB and TnsC—TnsA and TnsB were fused with an intervening NLS and 3×HA tag and an NLS was included at the internal S304 position (or corresponding position) in TnsC. Transposition was monitored by the mate-out assay. In the assay a mobilizable plasmid is a target for random transposition and a mini element (mini element=the left and right transposon ends flanking an antibiotic resistance marker for use as a genetic marker) resides at a neutral position in the chromosome. Transposition is monitored after inducing transposition with TnsA-NLS-3×HA-TnsB fusion and TnsC with Alt NLS inserted at S304 for Tn6900 or corresponding residue in the alignment for other elements. Altered TnsC and TnsAB are encoded as a synthetic operon in the TnsC-TnsAB order with an optimized ribosome loading site sequence inserted between on an expression plasmid (pBAD322) under an arabinose inducible promoter. A genetic marker on the mobile plasmid is used to mate the plasmid into a new host where a genetic marker on the transposon is used to determine the percent of the mobile plasmids that were targeted for transposition. Each example was tested three times with the mean+standard deviation graphed normalized to transposition frequency with Tn6900. Dashed bars indicate samples where transposition frequency exceeded the upper threshold of the experiment (TMTC—Too Many To Count). Some examples showed no transposition in the assay (Dead).

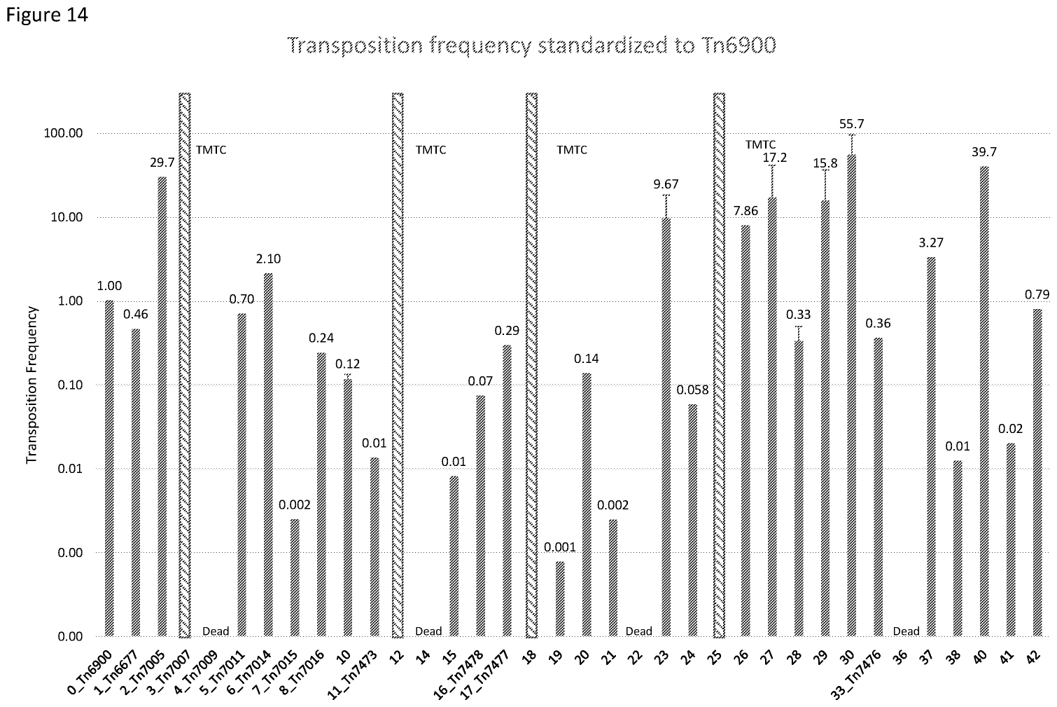

FIG. 15—Analysis of high activity transposons with Cascade with typical/atypical guide RNAs. Transposition was monitored by the mate-out assay. In the assay a mobilizable plasmid is a target for random transposition and a mini element (mini element=the left and right transposon ends flanking an antibiotic resistance gene for use as a genetic marker) resides at a neutral position in the chromosome. Transposition is monitored after inducing transposition with TnsA-NLS-3×HA-TnsB fusion and TnsC with Alt NLS inserted at S304 for Tn6900, or corresponding residue in the alignment for other elements. Altered TnsC and TnsAB of the following elements Tn6900, Tn6677, Tn7005, Tn7011 are encoded as a synthetic operon in the TnsC-TnsAB order with an optimized ribosome loading site sequence inserted between on an expression plasmid (pBAD322) under an arabinose inducible promoter. A genetic marker on the mobile plasmid is used to mate the plasmid into a new host where a genetic marker on the transposon is used to determine the percent of the mobile plasmids that were targeted for transposition. Each example was tested three times with the standard deviation shown. The transposition proteins were tested alone (noCascade) or in combination with TniQ, Cas8-5, Cas7, and Cas6 expressed in a synthetic operon with reading frames separated by optimized ribosome loading site sequences with wild-type untagged proteins-Q-Cascade and typical/atypical guide RNA combinations were expressed under arabinose control in a pCDF vector. The transposition frequency was monitored with the plasmid encoding transposition machinery and with or without the Q-Cascade and typical/atypical guide plasmids. The percentage in bold indicates the frequency of the on-target transposition event (on-target transposition inactivates the lacZ gene giving colonies that are white on media with X-gal indicator instead of blue).

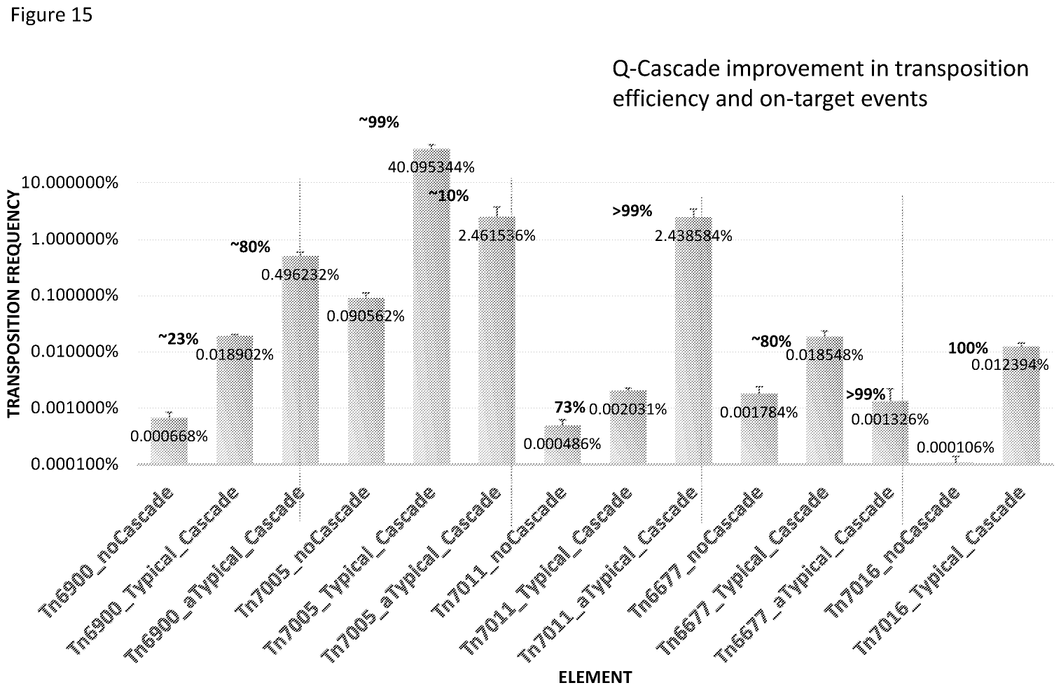

FIG. 16A shows a multiple sequence alignment of 36 full length TnsC protein sequences performed with Clustal Omega (clustalo Version 1.2.4) (Sievers F., Wilm A., Dineen D., Gibson T. J., Karplus K., Li W., Lopez R., McWilliam H., Remmert M., Söding J., Thompson J. D. and Higgins D. G. (2011) Fast, scalable generation of high-quality protein multiple sequence alignments using Clustal Omega. Mol. Syst. Biol. 7:539; the disclosure of which is incorporated herein by reference) for the sequences listed in the left column of the alignment. A portion of the sequence alignment corresponding to proposed insertion sites within TnsC is shown. Organism names and proteins of the disclosure for the TnsC protein sequences are as shown in Table A, which provides sequences for modified Wild type and modified TnsC proteins; Wild-type TnsA, Wild-type TnsB, Modified TnsAB fusion, Wild-type TnsC. Modified TnsC. Wild-type TniQ and Modified TniQ. For each individual aligned sequence the respective number of the first residue in the portion shown appears at the front of the sequence and the number of the last residue in the portion shown appears at the end of the sequence. Alignment adjustments are shown as dashes and added for convenience but do not represent additions, deletions, or gaps in the actual protein sequence. For reference, a consensus guide to #0-Tn6900 appears at the top and bottom of the alignment with the “!” corresponding to Y303, “$” corresponding to S304, and “@” corresponding Y306. Serine residues corresponding to S304 are underlined. The sequences from top to bottom in FIG. 16A are SEQ ID NO's 469-504.

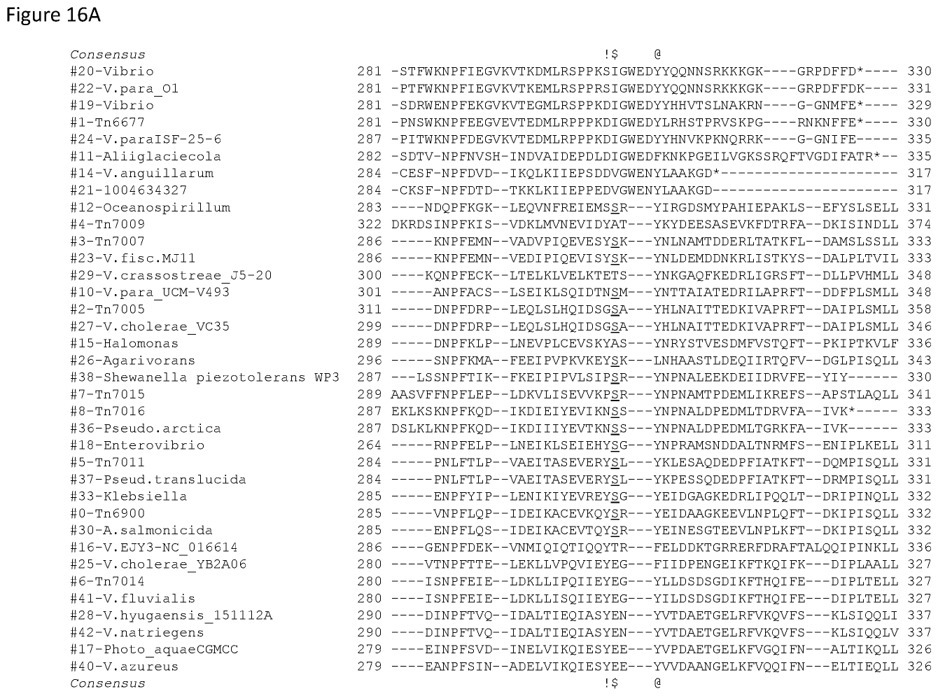

FIG. 16B shows a multiple sequence alignment of 28 full length TnsC protein sequences performed with Clustal Omega. A portion of the sequence alignment corresponding to proposed insertion sites within TnsC is shown. Nomenclature of the TnsC protein sequences is as shown in Table A. For each individual aligned sequence the respective number of the first residue in the portion shown appears at the front of the sequence and the number of the last residue in the portion shown appears at the end of the sequence. Alignment adjustments are shown as dashes and added for convenience but do not represent additions, deletions, or gaps in the actual protein sequence. For reference, a consensus guide to #0-Tn6900 appears at the top and bottom of the alignment with the “!” corresponding to Y303, “$” corresponding to S304, and “@” corresponding Y306. Serine residues corresponding to S304 are underlined. The sequences from top to bottom in FIG. 16B are SEQ ID NO:'s 505-532.

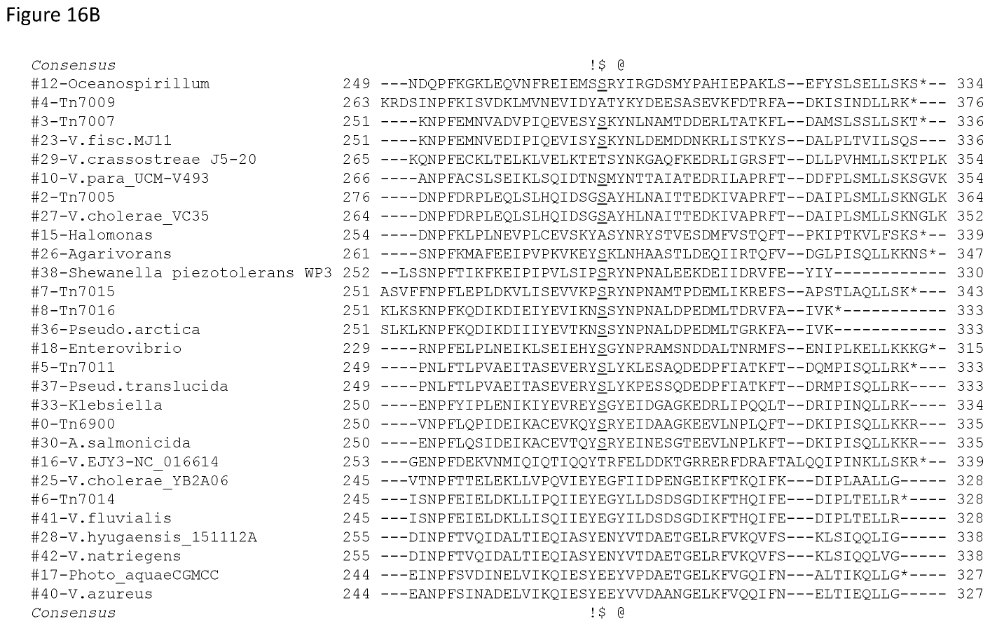

### DETAILED DESCRIPTION

Unless defined otherwise herein, all technical and scientific terms used in this disclosure have the same meaning as commonly understood by one of ordinary skill in the art to which this disclosure pertains.

Every numerical range given throughout this specification includes its upper and lower values, as well as every narrower numerical range that falls within it, as if such narrower numerical ranges were all expressly written herein.

The disclosure includes all polynucleotide and amino acid sequences described herein. Each RNA sequence includes its DNA equivalent, and each DNA sequence includes its RNA equivalent. Complementary and anti-parallel polynucleotide sequences are included. Every DNA and RNA sequence encoding polypeptides disclosed herein is encompassed by this disclosure. Amino acids of all protein sequences and all polynucleotide sequences encoding them are also included, including but not limited to sequences included by way of sequence alignments. Sequences of from 40.00%-99.99% identical to any sequence (amino acids and nucleotide sequences) of this disclosure are included.

The disclosure includes all polynucleotide and all amino acid sequences that are identified herein by way of a database entry. Such sequences are incorporated herein as they exist in the database on the filing date of this application or patent.

As used in the specification and the appended claims, the singular forms “a” “and” and “the” include plural referents unless the context clearly dictates otherwise. Ranges and other values may be expressed herein as from “about” or “approximately” one particular value, and/or to “about” or “approximately” another particular value. When values are expressed as approximations by the use of the antecedent “about” or “approximately” it will be understood that the particular value forms another embodiment. The term “about” and “approximately” in relation to a numerical value encompasses variations of +/−10%, to +/−1%.

The disclosure includes all steps and reagents such as proteins and nucleic acids, and all combinations of steps reagents, described herein, and as depicted on the accompanying figures. The described steps may be performed as described, including but not necessarily sequentially. Any described reagent(s) and step(s) may be excluded from the claims of this disclosure. As such, the described reagents, steps, and systems of this disclosure may comprise or consist of any one or combination of said reagents and steps. The disclosure also includes all periods of time and all temperatures described herein.

The disclosure includes the descriptions of PCT application no. PCT/US2020/22964, filed Mar. 16, 2020, published as PCT publication no. WO 2020/186262, and PCT application no. PCT/US21/22582, filed Mar. 16, 2021, published as PCT publication no. WO 2021/188553, the entire disclosures of each of which are incorporated herein by reference.

For any protein described herein that is encoded genetic information in a particular prokaryote, the disclosure includes homologous and orthologous proteins that are found in other prokaryotes. Such homologous and orthologous proteins can be modified at positions that can be determined by one skilled in the art based on demonstrations of modifications of proteins as described herein. In a non-limiting embodiment a reference sequence by which homologous, and orthologous proteins (i.e. orthologs), and amino acid positions within such proteins, can be identified is Aeromonas salmonicida strain S44, which may include plasmid pS44-1, and/or the Aeromonas salmonicida strain S44 and its Tn6900 element. Representative sources of proteins that can be modified are described herein including but not limited to figures and tables of this disclosure.

Modified proteins that are encompassed by this disclosure include proteins that can participate in modification of a DNA substrate as further described herein. Proteins that are modified may have at least 40%, 50%, 55%, 60%, 65%, 70%, 75%, 80%, 85%, 90%, 95%, 97%, 98%, 99%, or at least 99.5% amino acid sequence identity with a sequence described herein by way of a sequence identifier or reference to a database sequence. Percent sequence identity is defined as the percentage of amino acid residues in a particular sequence that are identical with the amino acid residues in a reference polypeptide sequence, after aligning the sequences and introducing gaps, if necessary, to achieve a maximum percent sequence identity. In one embodiment, a homologous protein has at least 80% sequence identity to a described sequence. In embodiments, an orthologous protein has 40% to 79% sequence identity to a described sequence. In embodiments, a homologous or orthologous protein is modified at an amino position that corresponds to a specific location of an amino acid sequence that is described herein.

The figures that form a part of this disclosure provide representative examples of constructs used for CRISPR-based engineering as described further below, and results obtained using the constructs. The disclosure includes each construct illustrated by the figures, each component of each construct individually, and all combinations thereof. A component of the described proteins may comprise a linker, a protein tag, a nuclear localization signal, and proteins that comprise any of: insertion of amino acids, replacement of amino acids, and addition of amino acids internally and on the N-terminus, C-terminus, and combinations thereof, thereby providing modified proteins

In embodiments, the modified proteins comprises one or more I-F3 proteins, which include I-F3 transposon proteins TnsA, TnsB, TnsC, TniQ, and I-F3b Cas proteins Cas8, Cas5, Cas7, and Cas6. Representative amino acid sequences for wild type and modified TnsA, TnsB, TnsC, TniQ, and TnsA-TnsB fusion proteins are provided in Table A. Representative amino acid sequences for wild type and modified Cas8, Cas5, Cas7, and Cas6 are shown in Table B, with Cas8/5 shown as a fusion protein as further described herein.

In non-limiting embodiments, the proteins of this disclosure comprise at least one protein that is from, or comprises modification of, one or more organisms that include any I-F3 transposons, including but not necessarily limited to the I-F3a and I-F3b subbranch of the I-F3 elements. Representative and non-limiting examples of I-F3 systems are described herein in the specification and the figures.

In embodiments, a protein is derived from an organism by, for example, expressing the protein using an expression vector, or an mRNA that is produced by a user of a described system for modifying a DNA template, as further described herein.

In embodiments, the modified proteins include but are not necessarily limited to TnsC protein, TnsA protein and TnsB protein.

The modifications may comprise insertions, substitutions, or amino acids that are added to the N-terminus or C-Terminus of the described proteins.

In an embodiment, the disclosure provides modified TnsC proteins that comprise an insertion or a replacement of endogenous amino acids. In embodiments, the insertion is internal to the TnsC protein. In embodiments, the replacement is a replacement of endogenous internal TnsC amino acids. By “endogenous” it is meant that a replacement comprises a replacement of a wild type amino acid sequence. By “internal” it is meant an insertion is not located at the C-terminus or N-terminus of the TnsC protein, although the disclosure includes TnsC and other proteins as described herein that have amino acids added to the C-terminus, N-terminus, or both. Insertions, replacements, and amino acid additions, are referred to herein as “modifications.” In non-limiting examples, a modification is made at a position that is at the N-terminus or C-terminus of a described protein. In an example a modification is at least one amino acid from an N or C terminus of a described protein, or at a position that is 2-400 amino acids from an N terminus or a C-terminus of a described protein. In one example a modification is made between amino acids acid 100 and 250 of a described protein. In one example a modification is made between amino acids 130-160 of a described protein. In embodiments, a modification is made between amino acids 140 and 150 of a described protein. In embodiments a modification is made N-terminal or C-terminal relative to position 100 of a described protein. In embodiments a modification is made N-terminal or to position 100 of a described protein. In embodiments a modification is made C-terminal relative to position 100 of a described protein. In embodiments a modification is made N-terminal or C-terminal relative to position 300 of a described protein. In embodiments an insertion is made at the amino acid immediately after or before amino acid 143, 145, or 146 of a described protein. In embodiments an insertion is made immediately after or immediately before after amino acid 303, 304, or 305 described herein. All of the modifications described above pertain and their amino acid positions apply to each and every protein described herein.

In an embodiment the disclosure provides a modified TniQ protein.

In an embodiment the disclosure provides a modified TnsA protein.

In an embodiment the disclosure provides a modified TnsB protein.

In an embodiment the disclosure provides an engineered fusion protein comprising a wild type or modified TnsA protein and a wild type or modified TnsB protein. An engineered fusion protein comprising a wild type TnsA and wild type TnsB protein of this disclosure is a fusion protein comprising TnsA and TnsB proteins that are not fused in an unmodified system, i.e., the TnsA and TnsB proteins are not produced as a single protein by naturally occurring bacteria. In embodiments a TnsA and TnsB fusion protein comprises an insertion of amino acids between the TnsA and TnsB components of the fusion protein.

In embodiments the disclosure comprises a modification of a Cas protein, including but not necessarily limited to Cas5, Cas6, Cas7, Cas8, or Cas8-5. With respect to Cas5 and Cas8, the Cas8 and Cas5 proteins can be found as a fusion protein in some naturally occurring bacteria. The fusion protein may be referred to herein as Cas8/5 or Cas8-5. Within the fusion protein the Cas8 segment, the Cas5 segment, or both may be modified as described herein, including but not limited to amino acid additions and substitutions, representative examples of which are provided in Table B.

In an embodiment the disclosure provides a modified TnsC protein that comprises an insertion in a segment comprising a sequence Xaa₁-Xaa₂-Xaa₃ wherein at least one of the amino acids is a Ser and at least one of the amino acids is a Tyr. In an embodiment one of the amino acids is Ser, one of the amino acids is a Tyr, and the third amino acid is any amino acid. In embodiments, the disclosure provides a modified TnsC protein with an insertion of amino acids beginning at or approximately at position 144 or 304, or a combination thereof, of a TnsC protein, or at a corresponding position in a homologous or orthologous protein. In embodiments, in an unmodified TnsC protein a Ser is present at position 304. In an unmodified the TnsC protein a Leu is at position 144. The stated TnsC positions can be taken in reference to proteins encoded by the Tn6900 element.

In embodiments the disclosure provides a combination of TnsA, TnsB, and TnsC, wherein at least one of the TnsA, TnsB, or the TnsC comprises an insertion or replacement of internal amino acids, and/or wherein the TnsA, and TnsB components are provided as an engineered fusion protein that optionally comprises an insertion between the TnsA and TnsB components. In embodiments, an insertion between a TnsA and TnsB protein is between amino acids 500-700 of the TnsA or TnsB protein.

In embodiments a modification comprises an insertion or replacement of one or more amino acids. In embodiments the modification comprises 2-30 amino acids. In embodiments, the modification comprises a randomized sequence. In embodiments, the modification comprises an introduced protein purification tag, non-limiting examples of which include FLAG-tags, streptavidin, V5 tags, a tag derived from the c-myc gene product (e.g., a myc tag), and the like. In embodiments, only one insertion, only one replacement, or only one addition is made. In embodiments, more than one insertion, replacement, or addition, or a combination thereof, is made. In embodiments, the replacement or insertion comprises linking amino acids that connect a first component to a second component. Suitable amino acid linkers may be mainly composed of relatively small, neutral amino acids, such as glycine, serine, and alanine, and can include multiple copies of a sequence enriched in glycine and serine. In specific and non-limiting embodiments, the linker comprises 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or more amino acids.

In embodiments, the modification comprises a nuclear localization sequence (NLS) that functions in trafficking the modified protein to the nucleus of a cell. Suitable NLS sequence are known in the art and can be adapted for use with the proteins described herein when given the benefit of the present disclosure.

In an embodiment, the NLS comprises an SV40 NLS. In embodiments, the NLS comprises a nucleoplasmin NLS. In embodiments, the NLS comprises the alternate (Alt) sequence. In embodiments, the

In embodiments, an insertion or replacement comprises any one or combination, of a repeated sequence in the following table, which also includes a representative linker:

| | |
|---|---|
| NLS (SV40) | PKKKRKV (SEQ ID NO: 533) |
| NLS (alternate) | PAAKKKKLD (SEQ ID NO: 539) |
| NLS | KRPAATKKAGQAKKKK (SEQ ID NO: 540) |
| (nucleoplasmid) | |
| 3xHA | YPYDVPDYAYPYDVPDYAYPYDVPDYA |
| | (SEQ ID NO: 541) |
| 3xmyc | EQKLISEEDLEQKLISEEDLEQKLISEEDL |
| | SEQ ID NO: 534 |
| GSG linker | GSG |

The constructs in the examples illustrated in the accompanying figures include the following sequences, in which the nuclear localization signal is shown in bold and the linker is shown in italics:

| | |
|---|---|
| NLS(SV40)- | M_PKKKRKV_GSG_TENRYFFAIRYLSDDVDCGLLAGRCISILHG |
| Cas6 | FRQAHPGIQIGVAFPEWSDRDLGRSIAFVSTNKSLLERFRERSYFQ |
| | VMQADNFFALSLVLEVPDTCQNVRFIRNQNLAKLFVGERRRRLA |
| | RAKRRAKARGEAFQPHMPDETKVVGVFHSVFMQSASSGQSYILH |
| | IQKHRYERSEDSGYSSYGLASNDLYTGYVPDLGAIFSTLF* |
| | (SEQ ID NO: 554) |
| NLS(SV40)- | M_PKKKRKV_GSG_ELCTHLSYSRSLSPGKAVFFYKTAESDFVPL |
| Cas7 | RIEVAKISGQKCGYTEGFDANLKPKNIERYELAYSNPQTIEACYV |
| | PPNVDELYCRFSLRVEANSMRPYVCSNPDVLRVMIGLAQAYQRLG |
| | GYNELARRYSANVLRGIWLWRNQYTQGTKIEIKTSLGSTYHIPDA |
| | RRLSWSGDWPELEQKQLEQLTSEMAKALSQPDIFWFADVTASLKT |
| | GFCQEIFPSQKFTERPDDHSVASRQLATVECSDGQLAACINPQKI |
| | GAALQKIDDWWANDADLPLRVHEYGANHEALTALRHPATGQDF |
| | YHLLTKAEQFVTVLESSEGGGVELPGEVHYLMAVLVKGGLFQKG |
| | KGR* (SEQ ID NO: 555) |
| NLS(SV40)- | M_PKKKRKV_GSG_VTIMHIEELLDIEDHGERDRQLRRYLAPYSA |
| Cas8-5 | EIGVDGAEKMALVVLLNLTLKRDRVESLCDEGLARQLLSDEGHIT |
| | NCLHTVRWLHTHNLKYPDARVSGERLIINAPPLIPGVISSAGLPMR |
| | MGWAHDSSDINLAKLFGTSFRYRDDSTNLALQLVARSKTWEQAL |
| | IGLGLTQQQLDIWCQLLASNLENNTFPTVVSPFSKQVRFLYQGNY |
| | CVVTPVVSHALLAQLQNVVHEKKLQCTYIHHDHPASVGSLVGAL |
| | GGKVAVLDYPPPVSPDKARSFSQARKHRLANGQSLFDRSVENDH |
| | VFIDALKHVISRPGLTRKQQRQLRLSALRYLRRQLAIWLGPIIEWR |
| | DEIVSSGRGEPGNLPSGGLELELITQPKKMLPELMLQVAGRFHLEL |
| | QNHSAGRRFAFHPALMAPIKSQILWLLRQLADDEEKDEPHPPTSC |
| | YYLHLSGLTVYDASALANPYLCGIPSLSALAGFCHDYERRLQSLI |
| | GQSVYFRGLAWYLGRYSLVTGKHLPEPSKSADPKSVSAIRRPGLL |
| | DGRYCDLGMDLIIEVHIPTGGSLPFTTCLDLLRVALPARFAGGCLH |
| | PPSLYEEYNWCTVYQDKSTLFTVLSRLPRYGCWIYPSDADLRSFE |
| | ELSEALALDRRLRPVATGFVFLEEPVERAGSIEGQHVYAESAIGTA |
| | LCINPVEMRLAGKKRFFGAGFWQLNDAKGAILMNGSANTG* |
| | (SEQ ID NO: 556) |
| TnsA- | MYRRHLKHSRVKNLFKFVSAKMNTVFTVESALEFDTCFHLEYSP |
| NLS(SV40)- | SVKFYEAQPEGFYYEFAGRQCPYTPDFRLVDQNDSVSFLEIKPSD |
| 3xHA-B | KVADPDFLHRFPLKQQRAIELSSPLKLVTEKQIRIAPILGNLKLLHR |
| | YSGFQSFTPLHMQLLGLVQKLGRVSLLRLSDSIDAPPEEVLASALS |
| | LIARGIMQSDLTVQKIGISSFVWAGGHSGIDHG_GSG_PKKKRKV_ |
| | GSG_YPYDVPDYAYPYDVPDYAYPYDVPDYA_GSG_MDKHNGG |
| | LFEDEFVIPQPSTSTSPIDAIQAVLPATVDSFPYVLKVEALHRRDY |
| | ILWVEKNLAGGWTEKNLTPLLADAALVLPPPTPNWRTLARWRKI |
| | YIQHGRKLVSLIPKHQAKGNARSRLPPSDELFFEQAVHRYLVGEQ |
| | PSIASAFQLYSDSIRIENLGVVENPIKTISYMAFYNRIKKLPAYQVM |
| | KSRKGSYIADVEFKAIASHKPPSRIMERVEIDHTPLDLLLLDDDLL |
| | VPLGRPSLTLLIDAYSHCVVGFNLNFNQPSYESVRNALLSSISKKD |
| | YVKNKYPSIEHEWPCYGKPETLVVDNGVEFWSASLAQSCLELGIN |
| | IQYNPVRKPWLKPMIERMFGIINRKLLEPIPGKTFSNIQEKGDYDP |
| | QKDAVMRFSTFLEIFHHWVIDVYHYEPDSRYRYIPIISWQHGNKD |
| | APPAPIIGDDLTKLEVILSLSLHCTHRRGGIQRYHLRYDSDELASY |
| | RMNYPDQTRGKRKVLVKLNPRDISYVYVFLEDLGSYIRVPCIDPI |
| | GYTKGLSLQEHQINVKLHRDFINEQMDVVSLSKARIYLNDRIKNE |
| | LIEVRRNIRQRNVKGVNKIAKYRNVGSHAETSIVHELNHPATNEVI |
| | SKMESASQPEHCDDWDNFTSGLEPY* (SEQ ID NO: 557) |
| TnsC- | MDLSCHDADKLRSFIECYVETPLLRAIQEDFDRLRFNKQFAGEPQ |
| NLS(SV40) | CMLLTGDTGTGKSSLIRHYAAKHPEQVRHGFIHKPLLVSRIPSRPT |
| | LESTMVELLKDLGQFGSSDRIHKSSAESLTEALIKCLKRCETELIIID |
| | EFQELIENKTREKRNQIANRLKYISETAKIPIVLVGMPWATKIAEEP |
| | QWSSRLLIRRSIPYFKLSDDRENFIRLIMGLANRMPFETQARLETK |
| | HTIYALFAACYGSLRALKQLLDESVKQALAAHAETLKHEHIAVA |
| | YALFYPDQVNPFLQPIDEIKACEVKQYSRYEIDAAGKEEVLNPLQF |
| | TDKIPISQLLKKR_GSG_PKKKRKV_* (SEQ ID NO: 558) |
| NLS(SV40)- | M_PKKKRKV_GSG_EQKLISEEDLEQKLISEEDLEQKLISEEDL_ |
| 3xmyc-TniQ | GSG_HLLVRPEPFADEALESYFLRLSQENGFERYRIFSGSVQDWL |
| | HTTDHAAAGAFPLELSRLNIFHASRSSGLRVRALQLVDRLTDGAP |
| | FRLLQLALCHSAISFGNHYKAVHRSGVDIPLSFIRVHQIPCCPDCLR |
| | ESAYVRQCWHFKPYVGCHRHGGRLIYSCPACGESLNYLASESINH |
| | CQCGFDLRTASTVPAQPDEIQLSALAYGCSFESSNPLLAIGSLSARF |
| | GALYWYQQRYLSDHEAVRDDRALTKAIGHFTAWPDAFWRELQQ |
| | MVDDALVRQTKPLNHTDFVDVFGSVVADCRQIPMRNTGQNFILK |
| | NLIGFLTDLVARHPQCRVANVGDLLLSAVDAATLLSTSVEQVRRL |
| | HHEGFLPLSIRPASRNTVSPHRAVFHLRHVVELRQARMQSHHDHS |
| | STYLPAW* (SEQ ID NO:559) |

In an embodiment, a protein of this disclosure comprises a contiguous sequence that comprises a linker. The linker may separate amino acid sequences of two distinct proteins that are joined in a fusion protein, or may be next to or flank a modification. One linker, or more than one linker may be used. Amino acid linkers may be mainly composed of relatively small, neutral amino acids, such as glycine, serine, and alanine, and can include multiple copies of a sequence enriched in glycine and serine. The linker may comprise from 1-100 amino acids, inclusive, and including all numbers and ranges of numbers there between. In specific and non-limiting embodiments, the linker comprises 3, 4, 5, 6, 7, 8, 9, 10, 11, 12, 13, 14, 15, 16, 17, 18, 19, and 20 amino acids. In a non-limiting embodiment, the linker comprises a segment of a protein from K. oxytoca. In an embodiment, the K. oxytoca linker comprises the sequence KYAQQNSLFICSFP (SEQ ID NO:547).

One or more of the proteins may be fused together, with or without other proteins. In embodiments, Cas8 and Cas5 are present in a single fusion protein.

In embodiments, TnsA and TnsB are present in a single fusion protein, as further described herein. In embodiments, the proteins are fused to one another without linking amino acids. In alternative embodiments, linking amino acids can be included. In embodiments, a fusion protein comprising TnsA and TnsB proteins also comprises an NLS.

In embodiments, proteins described herein may be expressed from a coding sequence that includes a ribosomal skipping sequence. Ribosomal skipping sequences are known in the art and include, in non-limiting embodiments, the ribosomal skipping peptides T2A, P2A, E2A, and F2A.

Representative fusion proteins comprising TnsA and TnsB, and modified TnsC proteins, have been constructed and determined to function for transposition in a standard mate out assay as demonstrated in the accompanying figures.

It will be apparent from the accompanying figures that only some modifications of the described protein result in improved transposition, e.g., more frequent insertion of a co-delivered DNA template. In embodiments, a CRISPR system that includes one or more of the described modified proteins exhibits higher transposition frequency than a control value. The control value may be a transposition frequency obtained using one or more modified proteins that comprises a different modification than the one or more modified proteins that exhibit a higher transposition frequency, as illustrated in the accompanying figures. The modified proteins of this disclosure may also exhibit less off-target transposition than a control value. In embodiments, the described modified proteins when used in a CRISPR system exhibit a gain-of-activity phenotype that permits transposition without a CRISPR-Cas effector.

In embodiments, the disclosure facilitates an increase of transposition efficiency relative to a control, such as transposition from a chromosome to a plasmid, or a plasmid to a chromosome, of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, fold greater than a control value. In a non-limiting embodiment, the control comprises transposition frequency exhibited by a system that uses unmodified proteins that are encoded by Aeromonas salmonicida strain S44.

Transposition efficiency can be determined for transposition events where the transposition comprises transposing an element in cis, e.g., transposition from one location in a chromosome to a different location in the same chromosome. In embodiments, an increase of transposition efficiency is obtained using a system comprising at least a first modified protein of this disclosure comprising an internal modification, relative to transposition efficiency of a system comprising the same first modified protein but with a different modification, such as an addition of amino acids at its N or C terminus.

In embodiments, the disclosure provides systems comprising the described modified proteins. The systems comprise one or more of the modified proteins, a guide RNA that is targeted to a selected location in a chromosome or plasmid, and a DNA cargo sequence.

Any suitable guide RNA may be used with the described modified protein. In embodiments, the guide RNA comprises atypical repeats, such guide RNAs being described in PCT application no. PCT/US2021/22582, from which the description of guide RNAs and atypical repeats, and all organisms, and proteins and CRISPR RNAs encoded by the organisms, is incorporated herein by reference.

The described systems also provide a DNA cargo sequence for use in insertion into a DNA substrate. The DNA cargo sequence can include left and right end transposon sequences. The transposon left and right end sequences may also be inserted with a DNA cargo. The DNA cargo sequence is inserted into a DNA substrate by cooperation of the described proteins and the targeting RNA to produce the DNA editing. Those skilled in the art will be able to understand the terms “left” and “right” transposon sequences, and recognize such sequences.

For use with I-F3 systems, the one or more I-F3 proteins may be obtained from, and modified, from any of organism that encode I-F3 proteins. In embodiments, an I-F3b protein that is used and/or modified according to this disclosure is encoded by the genome of an organism with an attachment site downstream of the ffs gene encoding the signal recognition particle, and those that are downstream of the downstream of the rsmJ gene.

In embodiments, the described modified proteins are obtained, or derived, from type any I-F3 systems, or type I-B Tn7-CRISPR-Cas systems.

The disclosure includes intact proteins described herein, and also includes functional fragments thereof. A “functional fragment” means one or more segments of contiguous amino acids of a polypeptide described herein which retain sufficient capability to participate in target RNA programmed insertion of the DNA insertion template. In embodiments, a functional fragment may therefore comprise or consist of, for example, a core domain, a catalytic domain, a polynucleotide binding domain, and the like. A single domain, or more than one domain, can be present in a functional fragment.

In embodiments, the compositions and methods of this disclosure are functional in a heterologous system. “Heterologous” as used herein means a system, e.g., a cell type, in which one or more of the components of the system are not produced without modification of the cells/system. A non-limiting embodiment of a heterologous system is any bacteria that is not Aeromonas salmonicida, including but not necessarily limited to Aeromonas salmonicida strain S44. In embodiments, a representative and non-limiting heterologous system is any type of E. coli. A heterologous system also includes any eukaryotic cell. In embodiments, the heterologous cell is a member of any group that does not endogenously use an I-F3b system.

In embodiments, the presently described systems are used to insert a DNA insertion template to virtually any position in a bacterial genome, any episomal element, or a eukaryotic chromosome, in an orientation dependent fashion, but in certain instances may require a PAM sequence. In embodiments, the system is targeted via a targeting RNA to a sequence in a chromosome in a eukaryotic cell, or to a DNA extrachromosomal element in a eukaryotic cell, such as a DNA viral genome. Thus, the disclosure includes modifying eukaryotic chromosomes, and eukaryotic extrachromosomal elements, such as DNA in any organelle. Accordingly, the type of extrachromosomal elements that can be modified according to the presently described compositions and methods are not particularly limited.

In embodiments, systems of this disclosure include a DNA cargo for insertion into a eukaryotic chromosome or extrachromosomal element, or in the case of prokaryotes, a chromosome or a plasmid. Thus, instead of transposing an existing segment of a genome in the manner in which transposons ordinarily function, the disclosure provides for insertion of DNA cargo that can be selected by the user of the system. The DNA cargo may be provided, for example, as a circular or linear DNA molecule. The DNA cargo can be introduced into the cell prior to, concurrently, or after introducing a system of the disclosure into a cell. The sequence of the DNA cargo is not particularly limited, other than a requirement for suitable right and left ends that are recognized by proteins of the system. The right and left end sequences that are required for recognition are typically from about 90-150-bp in length. As is known in the art, such 90-150 bp length comprises multiple 22 bp binding sites for the I-F3b TnsB transposase in the element in each of the ends that can be overlapping or spaced.

The minimum length of the DNA cargo is typically about 700 bp, but it is expected that from 700 bp to 120 kb can be used and inserted. The disclosure provides for insertion of a DNA cargo without making a double-stranded break, and without disrupting the existing sequence, except for residual nucleotides at the insertion site, as is known in the art for transposons. In embodiments, the insertion of the DNA cargo occurs at a position that is from approximately 47, 48, or 49 nucleotides from a protospacer in the target (e.g., chromosome or plasmid) sequence.

Without intending to be constrained by any particular theory, it is considered that, other than a requirement for certain sequences to function with the I-F3b sequences as described herein, the presently provided systems are agnostic with respect to the DNA sequence of the DNA insertion template. Accordingly, in embodiments, the DNA insertion template may be devoid of any sequence that can be transcribed, and as such may be transcriptionally inert. Such sequences may be used, for example, to alter a regulatory sequence in a genome, e.g., a promoter, enhancer, miRNA binding site, or transcription factor binding site, to result in knockout of an endogenous gene, or to provide an interval in the dsDNA substrate between two loci, and may be used for a variety of purposes, which include but are not limited to treatment of a genetic disease, enhancement of a desired phenotype, study of gene effects, chromatin modeling, enhancer analysis, DNA binding protein analysis, methylation studies, and the like.

In embodiments, the DNA sequence comprises a sequence that may be transcribed by any RNA polymerase, e.g., a eukaryotic RNA polymerase, e.g., RNA polymerase I, RNA polymerase II, or RNA polymerase III. In embodiments, the RNA that is transcribed may or may not encode a protein, or may comprise a segment that encodes a protein and a noncoding sequence that is functional. For example, functional RNAs include any catalytic RNA, or an RNA that can participate in an RNAi-mediated process. In embodiments, the functional RNA comprises all or a fragment of an siRNA, an shRNA, a tRNA, a spliceosomal RNA, or any type of micro RNA (miRNA), a snoRNA, or the like. In embodiments, the RNA that does not code for a protein encodes a long noncoding RNA (lncRNA).

In embodiments, the functional RNA may comprise a catalytic segment, and thus may be provided as a ribozyme. In embodiments, the ribozyme comprises a hammerhead ribozyme, a hairpin ribozyme, or a Hepatitis Delta Virus ribozyme. Such agents can be used, for example, to modulate any RNA to which they are targeted.

In embodiments, the DNA insertion template includes one or more promoters. The promoter may be constitutive or inducible. The promoter may be operably linked to a sequence that encodes any protein or peptide, or a functional RNA.

In embodiments, the DNA insertion template comprises one or more splice junctions. Thus, the insertion template may comprise a GU near a 5′ end of a coding sequence, and a branch site near the 3′ end of the coding sequence. In embodiments, the DNA insertion templates results in exon skipping, or it provides a mutually exclusive exon, or it provides an alternative 5′ splice junction as a donor site, or an alternative 3′ splice junction as an acceptor site, or a combination thereof. In embodiments, the DNA insertion template reduces or eliminates intron retention.

In embodiments, the DNA insertion template comprises at least one open reading frame, which may be operably linked to a promoter that is included with the DNA insertion template, or the DNA insertion template is linked to an endogenous cell promoter once integrated. The open reading frame, and thus the protein encoded by it, is not limited. In non-limiting embodiments, the DNA insertion template comprises an open reading frame that encodes a peptide, e.g., a peptide that can be translated and which may be, for example, from several to 50 amino acids in length, whereas longer sequences are considered proteins.

In embodiments, a protein encoded by the DNA insertion template includes a cellular localization signal, and thus may be transported to any particular cellular compartment. In embodiments, the encoded protein comprises a secretion signal. In embodiments, the encoded protein comprises a transmembrane domain, and thus may be trafficked to, and anchored in a cell membrane. In embodiments, the anchored protein may comprise either or both of an intracellular domain and an extracellular domain, and may accordingly be displayed on the cells surface, and may further participate in, for example, signal transduction, e.g., the protein comprises a surface receptor. In embodiments, a protein encoded by the DNA integrate template comprise a nuclear localization signal. In embodiments, a protein encoded by the DNA integrate template comprises one or more glycosylation sites.

In embodiments, the protein encoded by the DNA insertion template comprises at least one antigenic determinant, e.g., an epitope, and thus may be used to produce cells, such as antigen presenting cells, that may display a peptide comprising an epitope on the cell surface via MHC (e.g, HLA) presentation.

In embodiments, the protein encoded by the DNA insertion template encodes a binding partner, such as an antibody or antigen binding fragment of an antibody. In embodiments, the binding partner comprises an intact immunoglobulin, or as fragments of an immunoglobulin including but not necessarily limited to antigen-binding (Fab) fragments, Fab′ fragments, (Fab′)₂ fragments, Fd (N-terminal part of the heavy chain) fragments, Fv fragments (two variable domains), dAb fragments, single domain fragments or single monomeric variable antibody domains, isolated CDR regions, single-chain variable fragment (scFv), and other antibody fragments that retain antigen binding function. In embodiments, one or more binding partners are encoded by the DNA insertion template and encode all or a component of a Bi-specific T-cell engager (BiTE), a bispecific killer cell engager (BiKE), or a chimeric antigen receptor (CAR), such as for producing chimeric antigen receptor T cells (e.g. CAR T cells). In embodiments, the binding partners are multivalent, and as such may include tri-specific antibodies or other tri-specific binding partners.

In embodiments, the DNA insertion template encodes a T cell receptor, and thus may encode both an alpha and beta chain T cell receptor, or separate DNA insertion template s may be used.

In embodiments, the DNA insertion template encodes an enzyme; a structural protein; a signaling protein, a regulatory protein; a transport protein; a sensory protein; a motor protein; a defense protein; or a storage protein. In embodiments, the DNA insertion template encodes a protein or peptide hormone. In embodiments, the DNA insertion template encodes hemoglobin. In embodiments, the DNA insertion template encodes all or a segment of dystrophin. In embodiments, the DNA insertion template encodes a rod or cone protein. In embodiments, the DNA insertion template encodes a selectable or detectable marker. In embodiments, the detectable marker comprises a fluorescent protein, such as green fluorescent protein (GFP), enhanced GFP (eGFP), mCherry, and the like. In embodiments, the DNA insertion template encodes an auxotrophic marker, such as for use in yeast. In embodiments, the DNA insertion template encodes one or more proteins that are involved in a metabolic pathway.

In embodiments, the DNA insertion template encodes a peptide or protein that is intended to stimulate an immune response, which may be a humoral and/or cell mediated immune response, and may also include a peptide or protein that is intended to induce tolerance, such as in the case of an autoimmune disease or an allergy. In embodiments, the DNA insertion template encodes a Toll-like-receptor (TLR), or a TLR ligand, which may be an agonist or an antagonistic TLR ligand.

In embodiments, the DNA insertion template comprises a sequence that is intended to disrupt or replace a gene or a segment of a gene. Thus, the disclosure includes producing both knock in and knock out gene modifications in cells, and transgenic non-human animals that contain such cells, as well as prokaryotic cells modified in a similar manner.

In embodiments, the transposable DNA cargo sequence is inserted into the chromosome or extrachromosomal element within a 5 nucleotide sequence that includes the nucleotide that is located 47 nucleotides 3′ relative to the 3′ end of the protospacer. In embodiments, a DNA cargo insertion comprises an insertion at the center of a 5 bp target site duplication (TSD). Thus, in non-limiting embodiments, a suitable guide RNA directs an editing complex to a DNA target comprising a protospacer adjacent motif (PAM) that is cognate to the protospacer, so that precise integration of a DNA cargo can be achieved. In embodiments, the PAM comprises or consists of TACC or CC, NC, or CN (where “N” is any nucleotide). Thus, the location of the modification of DNA, such as insertion of a transposable DNA cargo sequence, is linked to the location of the PAM.

The I-F3b transposon and I-F3b Cas genes, or those from any other suitable system, can be expressed from any of a wide variety of existing mechanism that can replicate separately in the cell or be integrated into the host cell genome. Alternatively, they could be expressed transiently from an expression system that will not be maintained. In certain embodiments, the proteins themselves could be directly transformed into the host strain to allow their function. The disclosure allows for multiple copies of distinct transposon gene cassettes, multiple copies of Cas genes, CRISPR arrays, and multiple distinct cargo coding sequences to be introduced and to modify genetic material in the same cell. In embodiments a first set of I-F3b genes tnsA, tnsB, tnsC, and one or more I-F3b tniQ genes, and I-F3b Cas genes cas8f, cas5f, cas7f, and cas6f, and a sequence encoding at least a first guide RNA that is functional with I-F3b proteins encoded by the Cas genes, wherein at least one of the first set of I-F3b transposon genes, the I-F3b Cas genes, or the sequence encoding the first guide RNA are present within and/or are encoded by a recombinant polynucleotide that is introduced into heterologous bacteria, or eukaryotic cells. The disclosure thus includes second, third, fourth, fifth, or more copies of distinct I-F3b transposon genes, I-F3b Cas genes, and distinct cargo coding sequences.

The delivery vector can be based on any number of plasmid, bacteriophage or another genetic element, when used in prokaryotes. The vector can be engineered so it is maintained, or not maintained (using any number of existing plasmid, bacteriophage or other genetic elements). Delivery of these DNA constructions in bacteria can be by conjugation, bacteriophage or any transformation processes that functions in the bacterial host of interest.

Modifications of this system may include adapting the expression system to allow expression in eukaryotic or archaeal hosts. In embodiments, for eukaryotic cells, the disclosure includes use of at least one NLS in one or more proteins, as described herein and illustrated in the figures.

In embodiments, a system of this disclosure is introduced into eukaryotic cells using, for example, one or more expression vectors, or by direct introduction of ribonucleoproteins (RNPs). In embodiments, expression vectors comprise viral vectors. In embodiments, a viral expression vector is used. Viral expression vectors may be used as naked polynucleotides, or may comprises any of viral particles, including but not limited to defective interfering particles or other replication defective viral constructs, and virus-like particles. In embodiments, the expression vector comprises a modified viral polynucleotide, such as from an adenovirus, a herpesvirus, or a retrovirus, such as a lentiviral vector. In embodiments, a baculovirus vector may be used. In embodiments, any type of a recombinant adeno-associated virus (rAAV) vector may be used. In embodiments, a recombinant adeno-associated virus (rAAV) vector may be used. rAAV vectors are commercially available, such as from TAKARA BIOR and other commercial vendors, and may be adapted for use with the described systems, given the benefit of the present disclosure. In embodiments, for producing rAAV vectors, plasmid vectors may encode all or some of the well-known rep, cap and adeno-helper components. In certain embodiments, the expression vector is a self-complementary adeno-associated virus (scAAV). Suitable ssAAV vectors are commercially available, such as from CELL BIOLABS, INC.® and can be adapted for use in the presently provided embodiments when given the benefit of this disclosure.

Further modification of this approach can include expression and isolation of the proteins required for this process and carrying out some or all of the process in vitro to allow the assembly of novel DNA substrates. These DNA substrates can subsequently be delivered into living host cells or used directly for other procedures. Thus, the disclosure includes compositions, methods, vectors, and kits for use in the present approach to DNA editing.

In one example, the disclosure provides a system for modifying a genetic target in bacteria and/or eukaryotic cells. The system comprises a first set of I-F3b transposon genes tnsA, tnsB, tnsC, one or more I-F3b tniQ, Cas genes cas8f, cas5f, cas7f, and cas6f, wherein at least one of the proteins is modified as described herein, and a sequence encoding a guide RNA as described herein that is functional at least with proteins encoded by the I-F3b Cas genes, wherein at least one of the first set of transposon genes, the Cas genes, and/or or the sequence encoding the first guide RNA are present within and/or are encoded by a recombinant polynucleotide.

In embodiments, use of the described I-F3b systems exhibit a greater transposition frequency than transposition reference frequency. In embodiments, for instance in bacteria, transposition frequency can be determined using, for example, a bacteriophage (i.e. viral) vector that cannot replicate or integrate into the bacterial strain used in the assay. Therefore, while the viral vector injects its DNA into the cell, it is lost during cell replication. Encoded in the phage DNA is a miniature Tn7 element where the Right and Left ends of the element flank a gene that encodes resistance to an antibiotic, such as Kanamycin (KanR). If the transposon remains on the bacteriophage DNA the cell will still be killed by the antibiotic because the bacteriophage cannot be maintained in that particular strain of bacteria. However if the TnsA, TnsB, TnsC and other required I-F3b transposon proteins and nucleotide sequences described herein are added to the cell, transposition will occur because the transposon can move from the bacteriophage DNA into the chromosome (or plasmid) where it will be maintained and allow a colony of bacteria to grow that is antibiotic resistant. Therefore, when the number of infectious bacteriophage particles are in the assay is known, it permits calculation of a frequency of transposition as antibiotic resistant colonies of bacteria per bacteriophage used in the experiment. Thus, in embodiments, using one or a combination of the I-F3b proteins described herein increases transposition frequency. Accordingly, in some embodiments, one or more I-F3b proteins and guide RNA elements as described herein may be used to enhance CRISPR mediated insertion that is accompanied by the transposon-based constructs that are described herein.

In alternative embodiments, detectable markers and selection elements can be used. In embodiments, transposition frequency can be measured, for example, by a change in expression in a reporter gene. Any suitable reporter gene can be used, non-limiting examples of which include adaptations of standard enzymatic reactions which produce visually detectable readouts. In embodiments, adaptations of β-galactosidase (LacZ) assays are used. In embodiments, transposition of an element from one chromosomal location to another, or from a plasmid to a chromosome, or from a chromosome to a plasmid, results in a change in expression of a reporter protein, such as LacZ. In embodiments, use of a system described herein causes a change in expression of LacZ, or any other suitable marker, in a population of cells. In embodiments, transposition efficiency is determined by measuring the number of cells within a population that experience a transposition event, as determined using any suitable approach, such as by reporter expression, and/or by any other suitable marker and/or selection criteria. In embodiments, the disclosure provides for increased transposition, such as within a population of cells, relative to a control. As described above, the control can be any suitable control, such as a reference value, or any value using a control experiment with proteins that have different modifications. In embodiments, the reference value comprises a standardized curve(s), a cutoff or threshold value, and the like. In embodiments, transposition efficiency comprises use of a system of this disclosure to transpose all or a segment of DNA from one location to another within the same or separate chromosomes, from a chromosome to a plasmid, or from a plasmid or other DNA cargo to a chromosome. In embodiments, transposition efficiency is greater than a control value obtained or derived from transposition efficiency using the described system.

In one aspect, the disclosure provides a system for modifying a genetic target in one or more cells, the system comprising a first set of transposon genes tnsA, tnsB, tnsC, and tniQ, Cas genes cas8f, cas5f, cas7f, and cas6f, which encode at least one modified protein as described herein, and wherein at least two of said proteins are within a fusion protein, and a sequence encoding a guide RNA polynucleotide.

In another embodiment the disclosure provides a method comprising expressing a guide RNA in cells comprising transposon genes tnsA, tnsB, tnsC, wherein the encoded TnsC protein comprises a modification, and wherein and optionally the TnsA and TnsB proteins are present in a described fusion protein, non-limiting examples of which are provided by the Figures.

In certain approaches of this disclosure expression vectors, such as plasmids, are used to produce one or more than one construct and/or component of the system, and any of their cloning steps or intermediates. A variety of suitable expression vectors known in the art can be adapted to produce components of this disclosure, including vectors that contain any desirable cargo, but in the context of other components described herein, and atypical repeats.

In embodiments, any protein of this disclosure may be an Aeromonas salmonicida strain S44 protein, or a derivative thereof,

The disclosure allows for multiple copies of distinct transposon gene cassettes, multiple copies of Cas genes, CRISPR arrays, and multiple distinct cargo coding sequences to be introduced and to modify genetic material in the same cell. In embodiments a first set of transposon genes tnsA, tnsB, tnsC, and optionally one or more tniQ genes, Cas genes cas8f, cas5f, cas7f, and cas6f, and a sequence encoding a guide RNA that is functional with proteins encoded by the Cas genes, wherein at least one of the first set of transposon genes, the Cas genes, or the sequence encoding the first guide RNA are present within and/or are encoded by a recombinant polynucleotide that is introduced into bacteria, or eukaryotic cells. The disclosure thus includes second, third, fourth, fifth, or more copies of distinct transposon genes, Cas genes, and distinct cargo coding sequences

In one example, the disclosure provides a system for modifying a genetic target in bacteria and/or eukaryotic cells. The system comprises a first set of transposon genes tnsA, tnsB, tnsC, and optionally one or more tniQ, Cas genes cas8f, cas5f, cas7f, and cas6f, and a sequence encoding a first guide RNA, as described herein, that is functional with proteins encoded by the Cas genes, wherein at least one of the first set of transposon genes, the Cas genes, and/or or the sequence encoding the a guide RNA are present within and/or are encoded by a recombinant polynucleotide

In embodiments, the Tns proteins that are provided by this disclosure comprise mutations relative to a wild type sequence. A “wild type” sequence as used herein means a sequence that preexists in nature without experimentally engineering a change in the sequence. In embodiments, a wild type sequence is the sequence of a transposition element, a non-limiting example of which is the sequence of Aeromonas salmonicida strain S44 plasmid pS44-1, which can be accessed via accession no. CP022176 (Version CP022176.1), such as via www.ncbi.nlm.nih.gov/nuccore/CP022176.

Non-limiting embodiments of amino acid sequences comprising mutations and/or locations of mutations are described herein, and by way of the following amino acid sequences and accession numbers. Enlarged, bold and italicized amino acids signify non-limiting examples of mutations that are encompassed by this disclosure. Enlarged sequences are locations where other mutations may be made, and are also included in this disclosure. The disclosure includes amino acid insertions, replacements, and additions, to any of these sequences or their naturally occurring counterparts, the sequence of which are known in the art.

TnsA (A125D) change from Aeromonas salmonicida
strain S44 plasmid pS44-1 or TnsA(exact from
Aeromonas hydrophila strain AFG_SD03)
(SEQ ID NO: 548)
MYRRHLKHSRVKNLFKFVSAKMNTVFTVESALEFDTCFHLEYSP
SVKFYEAQPEGFYYEFAGRQCPYTPDFRLVDQNDSVSFLEIKPS
DKVADPDFLHRFPLKQQRAIELSSPLKLVTEKQIRL [symbol] PILGNLK
LLHRYSGFQSFTPLHMQLLGLVQKLGRVSLLRLSDSIDAPPEEV
LASALSLIARGIMQSDLTVQKIGISSFVWAGGHSGIDHG
TnsB (from Aeromonas salmonicida strain
S44 plasmid pS44-1)
(SEQ ID NO: 548)
MDKHNGGLFEDEFVIPQPSTSTSPIDAIQAVLPATVDSFPYVLK
VEALHRRDYILWVEKNLAGGWTEKNLTPLLADAALVLPPPTPNW
RTLARWRKIYIQHGRKLVSLIPKHQAKGNARSRLPPSDELFFEQ
AVHRYLVGEQPSIASAFQLYSDSIRIENLGVVEN [symbol] IKTISYMAF
YNRIKKLPAYQVMKSRKGSYIADVEFKAIASHKPPSRIMERVEI
DHTPLDLLLLDDDLLVPLGRPSLTLLIDAYSHCVVGFNLNFNQP
SYESVRNALLSSISKKDYVKNKYPSIEHEWPCYGKPETLVVDNG
VEFWSASLAQSCLELGINIQYNPVRKPWLKPMIERMFGIINRKL
LEPIPGKTFSNIQEKGDYDPQKDAVMRFSTFLEIFHHWVIDVYH
YEPDSRYRYIPIISWQHGNKDAPPAPIIGDDLTKLEVILSLSLH
CTHRRGGIQRYHLRYDSDELASYRMNYPDQTRGKRKVLVKLNPR
DISYVYVFLEDLGSYIRVPCIDPIGYTKGLSLQEHQINVKLHRD
FINEQMDVVSLSKARIYLNDRIKNELIEVRRNIRQRNVKGVNKI
AKYRNVGSHAETSIVHELNHPATNEVISKMESASQPEHCDDWDN
FTSGLEPY
TnsB (P167S) change from Aeromonas salmonicida
strain S44 plasmid pS44-1
(SEQ ID NO: 550)
MDKHNGGLFEDEFVIPQPSTSTSPIDAIQAVLPATVDSFPYVLK
VEALHRRDYILWVEKNLAGGWTEKNLTPLLADAALVLPPPTPNW
RTLARWRKIYIQHGRKLVSLIPKHQAKGNARSRLPPSDELFFEQ
AVHRYLVGEQPSIASAFQLYSDSIRIENLGVVEN [symbol] IKTISYMAF
YNRIKKLPAYQVMKSRKGSYIADVEFKAIASHKPPSRIMERVEI
DHTPLDLLLLDDDLLVPLGRPSLTLLIDAYSHCVVGFNLNFNQP
SYESVRNALLSSISKKDYVKNKYPSIEHEWPCYGKPETLVVDNG
VEFWSASLAQSCLELGINIQYNPVRKPWLKPMIERMFGIINRKL
LEPIPGKTFSNIQEKGDYDPQKDAVMRFSTFLEIFHHWVIDVYH
YEPDSRYRYIPIISWQHGNKDAPPAPIIGDDLTKLEVILSLSLH
CTHRRGGIQRYHLRYDSDELASYRMNYPDQTRGKRKVLVKLNPR
DISYVYVFLEDLGSYIRVPCIDPIGYTKGLSLQEHQINVKLHRD
FINEQMDVVSLSKARIYLNDRIKNELIEVRRNIRQRNVKGVNKI
AKYRNVGSHAETSIVHELNHPATNEVISKMESASQPEHCDDWDN
FTSGLEPY
TnsC (from Aeromonas salmonicida strain
S44 plasmid pS44-1)
(SEQ ID NO: 551)
MDLSCHDADKLRSFIECYVETPLLRAIQEDFDRLRFNKQFAGEP
QCMLLTGDTGTGKSSLIRHYAAKHPEQVRHGFIHKPLLVSRIPS
RPTLESTMVELLKDLGQFGSSDRIHKSSAESLTEALIKCLKRCE
TE [symbol] EFQELIENKTREKRNQIANRLKYISETAKIPIVLVGM
PWATKIAEEPQWSSRLLIRRSIPYFKLSDDRENFIRLIMGLANR
MPFETQARLETKHTIYALFAACYGSLRALKQLLDESVKQALAAH
AETLKHEHIAVAYALFYPDQVNPFLQPIDEIKACEVKQYSRYEI
DAAGKEEVLNPLQFTDKIPISQLLKKR
TnsC (E140A) change from Aeromonas salmonicida
strain S44 plasmid pS44-1
(SEQ ID NO: 552)
MDLSCHDADKLRSFIECYVETPLLRAIQEDFDRLRFNKQFAGEP
QCMLLTGDTGTGKSSLIRHYAAKHPEQVRHGFIHKPLLVSRIPS
RPTLESTMVELLKDLGQFGSSDRIHKSSAESLTEALIKCLKRCE
TE [symbol] FQELIENKTREKRNQIANRLKYISETAKIPIVLVGM
PWATKIAEEPQWSSRLLIRRSIPYFKLSDDRENFIRLIMGLANR
MPFETQARLETKHTIYALFAACYGSLRALKQLLDESVKQALAAH
AETLKHEHIAVAYALFYPDQVNPFLQPIDEIKACEVKQYSRYEI
DAAGKEEVLNPLQFTDKIPISQLLKKR
TnsC (E140Q) change from Aeromonas salmonicida
strain S44 plasmid pS44-1
(SEQ ID NO: 553)
MDLSCHDADKLRSFIECYVETPLLRAIQEDFDRLRFNKQFAGEP
QCMLLTGDTGTGKSSLIRHYAAKHPEQVRHGFIHKPLLVSRIPS
RPTLESTMVELLKDLGQFGSSDRIHKSSAESLTEALIKCLKRCE
TE [symbol] FQELIENKTREKRNQIANRLKYISETAKIPIVLVGMPWAT
KIAEEPQWSSRLLIRRSIPYFKLSDDRENFIRLIMGLANRMPFE
TQARLETKHTIYALFAACYGSLRALKQLLDESVKQALAAHAETL
KHEHIAVAYALFYPDQVNPFLQPIDEIKACEVKQYSRYEIDAAG
KEEVLNPLQFTDKIPISQLLKKR

In addition to any of the foregoing mutations, the disclosure also includes additional amino acid changes, such as changes in TnsC, which may include gain-of-activity mutations, in canonical Tn7 (e.g., homologous proteins), including but not necessarily limited to TnsABC (A225V), TnsABC (E233K), TnsABC (E233A), and TnsABC (E233Q).

Tables A and B provide representative examples of unmodified and modified protein sequences that are included within the scope of the disclosure.

TABLE A

| | | Wild- | Wild- | Modified | | | | |
|---|---|---|---|---|---|---|---|---|
| | | type | type | TnsAB | Wild-type | Modified | Wild-type | Modified |
| # | Qrganism | TnsA | TnsB | fusion | TnsC | TnsC | TniQ | TniQ |
| 0 | Tn6900 | MYRRH | MDKHN | MYRRHLKHS | MDLSCHDA | MDLSCHDA | MHLLVRPEP | MPKKKRKV |
| | | LKHSRV | GGLFED | RVKNLFKFVS | DKLRSFIECY | DKLRSFIEC | FADEALESYF | GSGEQKLIS |
| | | KNLFKF | EFVIPQ | AKMNTVFTV | VETPLLRAIQ | YVETPLLRAI | LRLSQENGF | EEDLEQKLI |
| | | VSAKM | PSTSTSP | ESALEFDTCF | EDFDRLRFN | QEDFDRLR | ERYRIFSGSV | SEEDLEQKL |
| | | NTVFTV | IDAIQA | HLEYSPSVKF | KQFAGEPQC | FNKQFAGE | QDWLHTTD | ISEEDLGSG |
| | | ESALEF | VLPATV | YEAQPEGFY | MLLTGDTGT | PQCMLLTG | HAAAGAFPL | HLLVRPEPF |
| | | DTCFHL | DSFPYV | YEFAGRQCP | GKSSLIRHYA | DTGTGKSSL | ELSRLNIFHA | ADEALESYF |
| | | EYSPSV | LKVEAL | YTPDFRLVD | AKHPEQVRH | IRHYAAKHP | SRSSGLRVRA | LRLSQENGF |
| | | KFYEAQ | HRRDYI | QNDSVSFLEI | GFIHKPLLVS | EQVRHGFI | LQLVDRLTD | ERYRIFSGS |
| | | PEGFYY | LWVEK | KPSDKVADP | RIPSRPTLEST | HKPLLVSRI | GAPFRLLQL | VQDWLHTT |
| | | EFAGRQ | NLAGG | DFLHRFPLK | MVELLKDLG | PSRPTLEST | ALCHSAISFG | DHAAAGAF |
| | | CPYTPD | WTEKN | QQRAIELSSP | QFGSSDRIH | MVELLKDL | NHYKAVHRS | PLELSRLNIF |
| | | FRLVDQ | LTPLLA | LKLVTEKQIRI | KSSAESLTEA | GQFGSSDRI | GVDIPLSFIR | HASRSSGLR |
| | | NDSVSF | DAALVL | APILGNLKLL | LIKCLKRCETE | HKSSAESLT | VHQIPCCPD | VRALQLVD |
| | | LEIKPSD | PPPTPN | HRYSGFQSF | LIIIDEFQELIE | EALIKCLKR | CLRESAYVR | RLTDGAPF |
| | | KVADPD | WRTLA | TPLHMQLLG | NKTREKRNQ | CETELIIIDEF | QCWHFKPY | RLLQLALCH |
| | | FLHRFP | RWRKIY | LVQKLGRVS | IANRLKYISET | QELIENKTR | VGCHRHGG | SAISFGNHY |
| | | LKQQRA | IQHGRK | LLRLSDSIDA | AKIPIVLVGM | EKRNQIAN | RLIYSCPACG | KAVHRSGV |
| | | IELSSPL | LVSLIPK | PPEEVLASAL | PWATKIAEE | RLKYISETAK | ESLNYLASESI | DIPLSFIRVH |
| | | KLVTEK | HQAKG | SLIARGIMQS | PQWSSRLLIR | IPIVLVGMP | NHCQCGFDL | QIPCCPDCL |
| | | QIRIAPI | NARSRL | DLTVQKIGIS | RSIPYFKLSD | WATKIAEE | RTASTVPAQ | RESAYVRQ |
| | | LGNLKL | PPSDEL | SFVWAGGH | DRENFIRLIM | PQWSSRLLI | PDEIQLSALA | CWHFKPYV |
| | | LHRYSG | FFEQAV | SGIDHGGSG | GLANRMPFE | RRSIPYFKLS | YGCSFESSNP | GCHRHGGR |
| | | FQSFTP | HRYLVG | PKKKRKVGS | TQARLETKH | DDRENFIRL | LLAIGSLSAR | LIYSCPACG |
| | | LHMQLL | EQPSIA | GYPYDVPDY | TIYALFAACY | IMGLANR | FGALYWYQ | ESLNYLASE |
| | | GLVQKL | SAFQLY | AYPYDVPDY | GSLRALKQLL | MPFETQAR | QRYLSDHEA | SINHCQCG |
| | | GRVSLL | SDSIRIE | AYPYDVPDY | DESVKQALA | LETKHTIYAL | VRDDRALTK | FDLRTASTV |
| | | RLSDSID | NLGVVE | AGSGMDKH | AHAETLKHE | FAACYGSLR | AIGHFTAWP | PAQPDEIQL |
| | | APPEEV | NPIKTIS | NGGLFEDEF | HIAVAYALFY | ALKQLLDES | DAFWRELQ | SALAYGCSF |
| | | LASALSL | YMAFY | VIPQPSTSTS | PDQVNPFLQ | VKQALAAH | QMVDDALV | ESSNPLLAI |
| | | IARGIM | NRIKKLP | PIDAIQAVLP | PIDEIKACEV | AETLKHEHI | RQTKPLNHT | GSLSARFGA |
| | | QSDLTV | AYQVM | ATVDSFPYVL | KQYSRYEIDA | AVAYALFYP | DFVDVFGSV | LYWYQQRY |
| | | QKIGISS | KSRKGS | KVEALHRRD | AGKEEVLNP | DQVNPFLQ | VADCRQIPM | LSDHEAVR |
| | | FVWAG | YIADVE | YILWVEKNL | LQFTDKIPIS | PIDEIKACE | RNTGQNFIL | DDRALTKAI |
| | | GHSGID | FKAIAS | AGGWTEKN | QLLKKR | VKQYSGSG | KNLIGFLTDL | GHFTAWP |
| | | HG (SEQ | HKPPSR | LTPLLADAAL | (SEQ ID | PAAKKKKL | VARHPQCRV | DAFWRELQ |
| | | ID | IMERVE | VLPPPTPNW | NO: 4) | DGSGRYEID | ANVGDLLLS | QMVDDAL |
| | | NO: 1) | IDHTPL | RTLARWRKI | | AAGKEEVL | AVDAATLLST | VRQTKPLN |
| | | | DLLLLD | YIQHGRKLVS | | NPLQFTDKI | SVEQVRRLH | HTDFVDVF |
| | | | DDLLVP | LIPKHQAKG | | PISQLLKKR | HEGFLPLSIR | GSVVADCR |
| | | | LGRPSL | NARSRLPPS | | (SEQ ID | PASRNTVSP | QIPMRNTG |
| | | | TLLIDAY | DELFFEQAV | | NO: 5) | HRAVFHLRH | QNFILKNLI |
| | | | SHCVVG | HRYLVGEQP | | | VVELRQAR | GFLTDLVAR |
| | | | FNLNFN | SIASAFQLYS | | | MQSHHDHS | HPQCRVAN |
| | | | QPSYES | DSIRIENLGV | | | STYLPAW* | VGDLLLSAV |
| | | | VRNALL | VENPIKTISY | | | (SEQ ID | DAATLLSTS |
| | | | SSISKKD | MAFYNRIKK | | | NO: 6) | VEQVRRLH |
| | | | YVKNKY | LPAYQVMKS | | | | HEGFLPLSI |
| | | | PSIEHE | RKGSYIADVE | | | | RPASRNTV |
| | | | WPCYG | FKAIASHKPP | | | | SPHRAVFH |
| | | | KPETLV | SRIMERVEID | | | | LRHVVELR |
| | | | VDNGV | HTPLDLLLLD | | | | QARMQSH |
| | | | EFWSAS | DDLLVPLGR | | | | HDHSSTYLP |
| | | | LAQSCL | PSLTLLIDAYS | | | | AW* (SEQ |
| | | | ELGINIQ | HCVVGFNLN | | | | ID NO: 7) |
| | | | YNPVRK | FNQPSYESV | | | | |
| | | | PWLKP | RNALLSSISK | | | | |
| | | | MIERM | KDYVKNKYP | | | | |
| | | | FGIINRK | SIEHEWPCY | | | | |
| | | | LLEPIPG | GKPETLVVD | | | | |
| | | | KTFSNI | NGVEFWSAS | | | | |
| | | | QEKGDY | LAQSCLELGI | | | | |
| | | | DPQKD | NIQYNPVRK | | | | |
| | | | AVMRF | PWLKPMIER | | | | |
| | | | STFLEIF | MFGIINRKLL | | | | |
| | | | HHWVI | EPIPGKTFSN | | | | |
| | | | DVYHYE | IQEKGDYDP | | | | |
| | | | PDSRYR | QKDAVMRF | | | | |
| | | | YIPIISW | STFLEIFHHW | | | | |
| | | | QHGNK | VIDVYHYEPD | | | | |
| | | | DAPPAP | SRYRYIPIISW | | | | |
| | | | IIGDDLT | QHGNKDAP | | | | |
| | | | KLEVILS | PAPIIGDDLT | | | | |
| | | | LSLHCT | KLEVILSLSLH | | | | |
| | | | HRRGGI | CTHRRGGIQ | | | | |
| | | | QRYHLR | RYHLRYDSD | | | | |
| | | | YDSDEL | ELASYRMNY | | | | |
| | | | ASYRM | PDQTRGKRK | | | | |
| | | | NYPDQ | VLVKLNPRDI | | | | |
| | | | TRGKRK | SYVYVFLEDL | | | | |
| | | | VLVKLN | GSYIRVPCID | | | | |
| | | | PRDISY | PIGYTKGLSL | | | | |
| | | | VYVFLE | QEHQINVKL | | | | |
| | | | DLGSYIR | HRDFINEQM | | | | |
| | | | VPCIDPI | DVVSLSKARI | | | | |
| | | | GYTKGL | YLNDRIKNEL | | | | |
| | | | SLQEHQ | IEVRRNIRQR | | | | |
| | | | INVKLH | NVKGVNKIA | | | | |
| | | | RDFINE | KYRNVGSHA | | | | |
| | | | QMDVV | ETSIVHELNH | | | | |
| | | | SLSKARI | PATNEVISK | | | | |
| | | | YLNDRI | MESASQPEH | | | | |
| | | | KNELIEV | CDDWDNFT | | | | |
| | | | RRNIRQ | SGLEPY* | | | | |
| | | | RNVKG | (SEQ ID | | | | |
| | | | VNKIAK | NO: 3) | | | | |
| | | | YRNVGS | | | | | |
| | | | HAETSI | | | | | |
| | | | VHELNH | | | | | |
| | | | PATNEV | | | | | |
| | | | ISKMES | | | | | |
| | | | ASQPEH | | | | | |
| | | | CDDWD | | | | | |
| | | | NFTSGL | | | | | |
| | | | EPY | | | | | |
| | | | (SEQ ID | | | | | |
| | | | NO: 2) | | | | | |
| 1 | Tn6677 | MTSLPT | MAKKG | MTSLPTPSAI | MSETREARIS | MSETREARI | MFLQRPKPY | MPKKKRKV |
| | | PSAITTS | FSSFHR | TTSALEYAFH | RAKRAFVST | SRAKRAFVS | SDESLESFFIR | GSGEQKLIS |
| | | ALEYAF | KAVSSQ | TPARNLTKSR | PSVRKILSYM | TPSVRKILSY | VANKNGYG | EEDLEQKLI |
| | | HTPARN | DTLESIE | GKNIHRYVS | DRCRDLSDL | MDRCRDLS | DVHRFLEAT | SEEDLEQKL |
| | | LTKSRG | VSSAN | VKMSKRITV | ESEPTCMM | DLESEPTC | KRFLQDIDH | ISEEDLGSG |
| | | KNIHRY | CLESVT | ESTLECDACY | VYGASGVGK | MMVYGAS | NGYQTFPTD | FLQRPKPYS |
| | | VSVKM | YQDISA | HFDFEPSIVR | TTVIKKYLNQ | GVGKTTVIK | ITRINPYSAK | DESLESFFIR |
| | | SKRITVE | FPETIAV | FCAQPIRFLY | NRRESEAGG | KYLNQNRR | NSSSARTASF | VANKNGYG |
| | | STLECD | EINFRLS | YLNGQSHSY | DIIPVLHIELP | ESEAGGDII | LKLAQLTFNE | DVHRFLEA |
| | | ACYHFD | ILRFLAR | VPDFLVQFD | DNAKPVDAA | PVLHIELPD | PPELLGLAIN | TKRFLQDID |
| | | FEPSIVR | KCETIV | TNEFVLYEVK | RELLVEMGD | NAKPVDAA | RTNMKYSPS | HNGYQTFP |
| | | FCAQPI | AKSIEP | SAYAKNKPD | PLALYETDLA | RELLVEMG | TSAVVRGAE | TDITRINPYS |
| | | RFLYYL | HRVELQ | FDVEWEAKV | RLTKRLTELIP | DPLALYETD | VFPRSLLRTH | AKNSSSART |
| | | NGQSH | QNYSRK | KAATELGLEL | AVGVKLIIIDE | LARLTKRLT | SIPCCPLCLRE | ASFLKLAQL |
| | | SYVPDF | PSAITIY | ELVEESDIRD | FQHLVEERS | ELIPAVGVK | NGYASYLW | TFNEPPELL |
| | | LVQFDT | RWWLA | TVVLNNLKR | NRVLTQVGN | LIIIDEFQHL | HFQGYEYCH | GLAINRTN |
| | | NEFVLY | FRKSDY | MHRYASKDE | WLKMILNKT | VEERSNRVL | SHNVPLITTC | MKYSPSTS |
| | | EVKSAY | NPISLAP | LNNVHNSLL | KCPIVIFGMP | TQVGNWL | SCGKEFDYR | AVVRGAEV |
| | | AKNKPD | NIKDRG | KIIKYNGAQS | YSKVVLQAN | KMILNKTKC | VSGLKGICCK | FPRSLLRTH |
| | | FDVEW | NRETKV | ARCLGEQLG | SQLHGRFSIQ | PIVIFGMPY | CKEPITLTSRE | SIPCCPLCLR |
| | | EAKVKA | STVVDSI | LKGRTVLPIL | VELRPFSYQ | SKVVLQAN | NGHEAACTV | ENGYASYL |
| | | ATELGL | MEQAV | CDLLSRCLLD | GGRGVFKTF | SQLHGRFSI | SNWLAGHES | WHFQGYEY |
| | | ELELVEE | ERVISG | TRLDKPLSLE | LEYLDKALPF | QVELRPFSY | KPLPNLPKSY | CHSHNVPLI |
| | | SDIRDT | RKVNVS | SRFELASYGG | EKQAGLANE | QGGRGVFK | RWGLVHW | TTCSCGKEF |
| | | VVLNNL | SAYKRV | SGPKKKRKV | SLQKKLYAFS | TFLEYLDKA | WMGIKDSEF | DYRVSGLK |
| | | KRMHR | RRKVRQ | GSGYPYDVP | QGNMRSLR | LPFEKQAGL | DHFSFVQFF | GICCKCKEP |
| | | YASKDE | YNLTHG | DYAYPYDVP | NLIYQASIEAI | ANESLQKKL | SNWPRSFHS | ITLTSRENG |
| | | LNNVH | TKYTYP | DYAYPYDVP | DNQHETITE | YAFSQGN | IIEDEVEFNLE | HEAACTVS |
| | | NSLLKI | KYESVR | DYAGSGAKK | EDFVFASKLT | MRSLRNLIY | HAVVSTSELR | NWLAGHES |
| | | KYNGA | KRVKKK | GFSSFHRKA | SGDKPNSW | QASIEAIDN | LKDLLGRLFF | KPLPNLPKS |
| | | QSARCL | TPFELLA | VSSQDTLESI | KNPFEEGVE | QHETITEED | GSIRLPERNL | YRWGLVH |
| | | GEQLGL | AGKGER | ELVSSANCLE | VTEDMLRPP | FVFASKLTS | QHNIILGELL | WWMGIKD |
| | | KGRTVL | VAKREF | SVTYQDISAF | PKDIGWEDY | GDKPNSW | CYLENRLWQ | SEFDHFSFV |
| | | PILCDLL | RRMGK | PETIAVEINF | LRHSTPRVSK | KNPFEEGV | DKGLIANLK | QFFSNWPR |
| | | SRCLLD | KILTSSV | RLSILRFLARK | PGRNKNFFE | EVTEDMLR | MNALEATV | SFHSIIEDEV |
| | | TRLDKP | LERVEID | CETIVAKSIEP | * (SEQ ID | PPPKDIGSG | MLNCSLDQI | EFNLEHAV |
| | | LSLESRF | HTVVDL | HRVELQQNY | NO: 11) | PAAKKKKL | ASMVEQRIL | VSTSELRLK |
| | | ELASYG | FAVHEE | SRKIPSAITIY | | DGSGGWE | KPNRKSKPN | DLLGRLFFG |
| | | * (SEQ | YRIPLGR | RWWLAFRK | | DYLRHSTPR | SPLDVTDYLF | SIRLPERNL |
| | | ID | PWLTQL | SDYNPISLAP | | VSKPGRNK | HFGDIFCLW | QHNIILGEL |
| | | NO: 8) | VDCYSK | NIKDRGNRE | | NFFE* (SEQ | LAEFQSDEF | LCYLENRL |
| | | | AVIGFYL | TKVSTVVDSI | | ID NO: 12) | NRSFYVSRW | WQDKGLIA |
| | | | GFEPPS | MEQAVERVI | | | * (SEQ ID | NLKMNALE |
| | | | YVSVSL | SGRKVNVSS | | | NO: 13) | ATVMLNCS |
| | | | ALKNAI | AYKRVRRKV | | | | LDQIASMV |
| | | | QRKDDL | RQYNLTHGT | | | | EQRILKPNR |
| | | | ISSYESIE | KYTYPKYESV | | | | KSKPNSPLD |
| | | | NEWLC | RKRVKKKTPF | | | | VTDYLFHFG |
| | | | YGIPDLL | ELLAAGKGE | | | | DIFCLWLAE |
| | | | VTDNG | RVAKREFRR | | | | FQSDEFNR |
| | | | KEFLSK | MGKKILTSSV | | | | SFYVSRW* |
| | | | AFDQA | LERVEIDHTV | | | | (SEQ ID |
| | | | CESLLIN | VDLFAVHEE | | | | NO: 14) |
| | | | VHQNK | YRIPLGRPWL | | | | |
| | | | VETPDN | TQLVDCYSK | | | | |
| | | | KPHVER | AVIGFYLGFE | | | | |
| | | | NYGTIN | PPSYVSVSLA | | | | |
| | | | TSLLDD | LKNAIQRKD | | | | |
| | | | LPGKSF | DLISSYESIEN | | | | |
| | | | SQYLQR | EWLCYGIPD | | | | |
| | | | EGYDSV | LLVTDNGKE | | | | |
| | | | GEATLT | FLSKAFDQA | | | | |
| | | | LNEIREI | CESLLINVHQ | | | | |
| | | | YLIWLV | NKVETPDNK | | | | |
| | | | DIYHKK | PHVERNYGT | | | | |
| | | | PNQRG | INTSLLDDLP | | | | |
| | | | TNCPNV | GKSFSQYLQ | | | | |
| | | | AWKKG | REGYDSVGE | | | | |
| | | | CQEWE | ATLTLNEIREI | | | | |
| | | | PEEFSG | YLIWLVDIYH | | | | |
| | | | SKDELD | KKPNQRGTN | | | | |
| | | | FKFAIV | CPNVAWKK | | | | |
| | | | DYKQLT | GCQEWEPEE | | | | |
| | | | KVGITV | FSGSKDELDF | | | | |
| | | | YKELSYS | KFAIVDYKQL | | | | |
| | | | NDRLAE | TKVGITVYKE | | | | |
| | | | YRGKKG | LSYSNDRLAE | | | | |
| | | | NHKVQ | YRGKKGNHK | | | | |
| | | | FKYNPE | VQFKYNPEC | | | | |
| | | | CMAVI | MAVIWVLD | | | | |
| | | | WVLDE | EDMNEYFTV | | | | |
| | | | DMNEY | NAIDYEYASR | | | | |
| | | | FTVNAI | VSLWQHKY | | | | |
| | | | DYEYAS | NMKYQAEL | | | | |
| | | | RVSLW | NSAEYDEDK | | | | |
| | | | QHKYN | EIDAEIKIEEI | | | | |
| | | | MKYQA | ADRSIVKTNK | | | | |
| | | | ELNSAE | IRARRRGAR | | | | |
| | | | YDEDKE | HQENSARAK | | | | |
| | | | IDAEIKI | SISNANPASI | | | | |
| | | | EEIADR | QKHEDEIVS | | | | |
| | | | SIVKTN | ADNDDWDI | | | | |
| | | | KIRARR | DYV* (SEQ | | | | |
| | | | RGARH | ID NO: 10) | | | | |
| | | | QENSAR | | | | | |
| | | | AKSISN | | | | | |
| | | | ANPASI | | | | | |
| | | | QKHEDE | | | | | |
| | | | IVSADN | | | | | |
| | | | DDWDI | | | | | |
| | | | DYV* | | | | | |
| | | | (SEQ ID | | | | | |
| | | | NO: 9) | | | | | |
| 2 | Tn7005 | MFDQT | MPPDS | MFDQTKKSS | MTMILKILKG | MTMILKILK | MKTDIQHYS | MPKKKRKV |
| | | KKSSHV | NSIFGFF | HVHNICKFM | ISLNLTPKQL | GISLNLTPK | DESLESFLLRL | GSGEQKLIS |
| | | HNICKF | DEFEAS | SLKNDAVVR | EQLKSFETCF | QLEQLKSFE | SQEQGYERF | EEDLEQKLI |
| | | MSLKN | EEESQL | TLSILEFDFCF | IEYPAITEIYSI | TCFIEYPAIT | SHFAEDIWF | SEEDLEQKL |
| | | DAVVRT | LPKELIL | HLEYNPNIKS | FDQLRFNHS | EIYSIFDQLR | DTMEQHEAI | ISEEDLGSG |
| | | LSILEFD | EPVEISS | FTSQPFGFH | LGGEPESFLL | FNHSLGGE | AGAFPLELN | KTDIQHYSD |
| | | FCFHLE | TIDSLPA | YLENNRKCR | TGEAGSGKT | PESFLLTGE | RINIYHAQTT | ESLESFLLRL |
| | | YNPNIK | KIQEEVL | YTPDFLAIGH | ALINNYLSRF | AGSGKTALI | SQMRVRVLI | SQEQGYER |
| | | SFTSQP | RRIKVIT | NEQSTFFEV | QSGSTWGK | NNYLSRFQ | HLENQLKLN | FSHFAEDI |
| | | FGFHYL | FVEKRL | KHSSQIPKPD | QPVLSTRVPS | SGSTWGKQ | NFGVLRLALS | WFDTMEQ |
| | | FNNRKC | KGGWT | FRERFEEKQR | RINEQNTLT | PVLSTRVPS | HSKAQFSPE | HEAIAGAFP |
| | | RYTPDF | EKNLNP | VALSEFNRRL | QFLVDLDCK | RINEQNTLT | YKAVHRLGS | LELNRINIY |
| | | LAIGHN | ILSLVES | VLVTEKQIR | SGGRGIRRR | QFLVDLDC | DYPFVFLGKR | HAQTTSQ |
| | | EQSTFF | ELQLTP | MGPTLDNFK | NEIALGEAVV | KSGGRGIRR | FTPICPLCISE | MRVRVLIH |
| | | EVKHSS | PSWRT | LLHRYSGLRT | KQLKRKSVEL | RNEIALGEA | APYIRQQW | LENQLKLN |
| | | QIPKPD | VATWK | VTEFQKRVL | IIVNEIQELVE | VVKQLKRK | QFLSQQACE | NFGVLRLAL |
| | | FRERFE | KSYAEA | AFIQRKQMV | FSTAEQRQVI | SVELIIVNEI | RHGCKLVHH | SHSKAQFSP |
| | | EKQRVA | GREASA | KLQEVSLYFG | ANTFKYMSE | QELVEFSTA | CPECQSRLEY | EYKAVHRL |
| | | LSEFNR | LIPKHTF | LSEQDTLISTL | EARVSFVLV | EQRQVIAN | QTTESISQCE | GSDYPFVFL |
| | | RLVLVT | KGNRQ | PWISSGHVK | GMPYADVIA | TFKYMSEE | CGFELRNSP | GKRFTPICP |
| | | EKQIRM | KEMDS | TDLNTIGFGL | TEPQWNSRL | ARVSFVLV | VEDAPVAAL | LCISEAPYIR |
| | | GPTLDN | QSLIDE | ETCVWCGS | SWRRKIDYF | GMPYADVI | LVARWLSGN | QQWQFLS |
| | | FKLLHR | AIQNVY | GPKKKRKVG | KLLKANSHSS | ATEPQWNS | DSKPLGLLKA | QQACERHG |
| | | YSGLRT | LTRERLS | SGYPYDVPD | KTASYGFDLE | RLSWRRKI | EMTLSERYG | CKLVHHCP |
| | | VTEFQK | VAEAYR | YAYPYDVPD | QKKHFARFV | DYFKLLKAN | FLLWYVNRY | ECQSRLEYQ |
| | | RVLAFI | YYKSRVI | YAYPYDVPD | AGLSSRMGF | SHSSKTASY | GDIENISFESF | TTESISQCE |
| | | QRKQM | QMNRG | YAGSGLTMP | DEPPVLTKN | GFDLEQKK | VEYCSCWPR | CGFELRNSP |
| | | VKLQEV | IVEGKIK | PDSNSIFGFF | ELLYPLFAMC | HFARFVAG | VLKEELDELV | VEDAPVAA |
| | | SLYFGLS | PIAERSF | DEFEASEEES | RGECRALKH | LSSRMGFD | NKADLIRIKD | LLVARWLS |
| | | EQDTLIS | YNRINE | QLLPKELILEP | FLKDALLTSF | EPPVLTKNE | WKKTFFNEV | GNDSKPLG |
| | | TLPWIS | LPPYEV | VEISSTIDSLP | NDNADTIDK | LLYPLFAMC | FGALLKDCR | LLKAEMTLS |
| | | SGHVKT | AIARFG | AKIQEEVLRR | AILSRTFAFKF | RGECRALK | QLPSRQLEC | ERYGFLLW |
| | | DLNTIG | KRYADR | IKVITFVEKRL | PYLDNPFDR | HFLKDALLT | NSVLTQVLA | YVNRYGDIE |
| | | FGLETC | EYRSVG | KGGWTEKN | PLEQLSLHQI | SENDNADTI | YFTKLMAAIP | NISFESFVEY |
| | | VWC* | QQVVA | LNPILSLVESE | DSGSAYHLN | DKAILSRTF | SSSKGNVGD | CSCWPRVL |
| | | (SEQ ID | TKPMEF | LQLTPPSWR | AITTEDKIVA | AFKFPYLDN | VLLSPLEAST | KEELDELVN |
| | | NO: 15) | VEIDHT | TVATWKKSY | PRFTDAIPLS | PFDRPLEQL | LLSCTTDEVY | KADLIRIKD |
| | | | PVPVILI | AEAGREASA | MLLSKNGLK | SLHQIDSGS | RLYEFGEIKA | WKKTFFNE |
| | | | DDELDI | LIPKHTFKGN | A* (SEQ ID | GSGPAAKK | AIRPRMHTKI | VFGALLKDC |
| | | | PLGRPY | RQKEMDSQ | NO: 18) | KKLDGSGA | ASHESAFTLR | RQLPSRQLE |
| | | | LTMLYD | SLIDEAIQNV | | YHLNAITTE | SVIETKLTRM | CNSVLTQV |
| | | | RFSKCIV | YLTRERLSVA | | DKIVAPRFT | CSENDGLSV | LAYFTKLM |
| | | | GCSINF | EAYRYYKSRV | | DAIPLSMLL | YLPEW* | AAIPSSSKG |
| | | | REPSFD | QMNRGIVE | | SKNGLKA* | (SEQ ID | NVGDVLLS |
| | | | SVRKAL | GKIKPIAERSF | | (SEQ ID | NO: 20) | PLEASTLLS |
| | | | LNSLLD | YNRINELPPY | | NO: 19) | | CTTDEVYRL |
| | | | KSWLKA | EVAIARFGKR | | | | YEFGEIKAAI |
| | | | KYPSIEN | YADREYRSV | | | | RPRMHTKI |
| | | | EWPCH | GQQVVATKP | | | | ASHESAFTL |
| | | | GKIDCL | MEFVEIDHT | | | | RSVIETKLTR |
| | | | VVDNG | PVPVILIDDE | | | | MCSENDGL |
| | | | AEFWS | LDIPLGRPYL | | | | SVYLPEW* |
| | | | QSLEDS | TMLYDRFSK | | | | (SEQ ID |
| | | | LRPLVS | CIVGCSINFR | | | | NO: 21) |
| | | | DIQYSQ | EPSFDSVRKA | | | | |
| | | | AAKPW | LLNSLLDKS | | | | |
| | | | RKSGIEK | WLKAKYPSIE | | | | |
| | | | LFDQM | NEWPCHGKI | | | | |
| | | | NKGLV | DCLVVDNGA | | | | |
| | | | NALPGK | EFWSQSLED | | | | |
| | | | TFTNPT | SLRPLVSDIQ | | | | |
| | | | QLQDY | YSQAAKPW | | | | |
| | | | NPKKDA | RKSGIEKLFD | | | | |
| | | | VVRVSV | QMNKGLVN | | | | |
| | | | FLELLHK | ALPGKTFTN | | | | |
| | | | WIVDYY | PTQLQDYNP | | | | |
| | | | HMAPD | KKDAVVRVS | | | | |
| | | | SREREIP | VFLELLHKWI | | | | |
| | | | YHKWH | VDYYHMAP | | | | |
| | | | QSKWT | DSREREIPYH | | | | |
| | | | PSYYDG | KWHQSKWT | | | | |
| | | | AEKEQL | PSYYDGAEK | | | | |
| | | | RVELGL | EQLRVELGLL | | | | |
| | | | LRHRTI | RHRTIGVAGI | | | | |
| | | | GVAGIR | RLHNLRYQS | | | | |
| | | | LHNLRY | AELIEYRKYC | | | | |
| | | | QSAELIE | TPNNGKQLF | | | | |
| | | | YRKYCT | VKTKTDPSDI | | | | |
| | | | PNNGK | SYIHVYLESE | | | | |
| | | | QLFVKT | KKYIKVPAVD | | | | |
| | | | KTDPSD | NSGYTNGLS | | | | |
| | | | ISYIHVY | LFEHQRIQKV | | | | |
| | | | LESEKKY | RRLNTKDLA | | | | |
| | | | IKVPAV | DDEALADTF | | | | |
| | | | DNSGYT | LYMKKRIHEE | | | | |
| | | | NGLSLF | TDRFRRVKSS | | | | |
| | | | EHQRIQ | KPNLPKTGN | | | | |
| | | | KVRRLN | TSRLAKFND | | | | |
| | | | TKDLAD | VGSEGPNSI | | | | |
| | | | DEALAD | NVTPVRLKSE | | | | |
| | | | TFLYMK | VVSDASEYL | | | | |
| | | | KRIHEET | DDDDFEDIE | | | | |
| | | | DRFRRV | GY* (SEQ ID | | | | |
| | | | KSSKPN | NO: 17) | | | | |
| | | | LPKTGN | | | | | |
| | | | TSRLAK | | | | | |
| | | | FNDVGS | | | | | |
| | | | EGPNSI | | | | | |
| | | | NVTPVR | | | | | |
| | | | LKSEVV | | | | | |
| | | | SDASEY | | | | | |
| | | | LDDDDF | | | | | |
| | | | EDIEGY | | | | | |
| | | | * (SEQ | | | | | |
| | | | ID | | | | | |
| | | | NO: 16 | | | | | |
| 3 | Tn7007 | MYVRTL | MDIEFP | MYVRTLKQS | MHTLSSTQK | MHTLSSTQ | MLNPIELYED | MPKKKRKV |
| | | KQSQVK | FTDEFQ | QVKNISKFM | EQLISFNQCF | KEQLISFNQ | ESLESCLLRIS | GSGEQKLIS |
| | | NISKFM | KILTTQS | SLKNDSIIRTE | IEYPIITHIYSI | CFIEYPIITHI | QNNCYDSFQ | EEDLEQKLI |
| | | SLKNDSI | YPAEIT | SMLEFDMCF | FNDLRMNQ | YSIFNDLRM | DFSDEVWF | SEEDLEQKL |
| | | IRTESM | NDKKTE | HLEYSPDVVS | GLGAEPQC | NQGLGAEP | QVKEEDREV | ISEEDLGSG |
| | | LEFDMC | VLTPSL | FESQPQGFH | MLLLGDTGS | QCMLLLGD | RGTFPATLN | LNPIELYED |
| | | FHLEYS | DSYDDA | YEYQGKRLP | GKSALINNYL | TGSGKSALI | TVNIYHSHTS | ESLESCLLRI |
| | | PDVVSF | IKAEVLR | YTPDFLITHS | LRQPPSNFS | NNYLLRQP | SDLKLKALIKI | SQNNCYDS |
| | | ESQPQ | RISFLR | SGQQQLLEV | ALSSLPVLHT | PSNFSALSS | EQWLEINNS | FQDFSDEV |
| | | GFHYEY | WIKPRL | KPLSKTQCP | RIPRRVNNE | LPVLHTRIP | PLLKSALSRS | WFQVKEED |
| | | QGKRLP | KGGWT | DFQSKFIQK | QTMYQLLTD | RRVNNEQT | SSTFLRQHSA | REVRGTFP |
| | | YTPDFLI | EKNLTP | QQAAQKLNL | LGQSPSGSR | MYQLLTDL | VFRNGVDIP | ATLNTVNIY |
| | | THSSGQ | LLNDAE | SLILITEKQIR | RAKRSEIALA | GQSPSGSR | RILLRKNGIP | HSHTSSDLK |
| | | QQLLEV | IDLKVSA | TGHLLNNFK | EGVVRALKR | RAKRSEIAL | VCPECLKENE | LKALIKIEQ |
| | | KPLSKT | PKWRTL | LLHRYAGLHS | KKTELIIINEF | AEGVVRAL | YIRQEWHFIT | WLEINNSPL |
| | | QCPDF | AEWHK | SATQKSIINL | QELIEFSSAK | KRKKTELIII | HDVCTRHKI | LKSALSRSS |
| | | QSKFIQ | NYHKSG | IQTVNKIQIN | ERQNVANTL | NEFQELIEF | GLLHHCPEC | STFLRQHSA |
| | | KQQAA | EKVSSLI | QJAHRLNISN | KYISEEARVSI | SSAKERQN | KASINYQKIE | VFRNGVDI |
| | | QKLNLS | PKHSHK | GEVLAGVLS | VLVGMPYA | VANTLKYIS | NITVCQCGF | PRILLRKNGI |
| | | LILITEK | GNKNM | WLSKGTLQT | DIIAEEPQW | EEARVSIVL | KFSDHLAPQ | PVCPECLKE |
| | | QIRTGH | NTDSDF | VYTNEMING | GSRLTWKTQ | VGMPYADII | ANSNALLIA | NEYIRQEW |
| | | LLNNFK | LITKAIN | NSIVSLGSGP | IEYFSLKNDM | AEEPQWGS | QWLNGENT | HFITHDVCT |
| | | LLHRYA | EKYLTL | KKKRKVGSG | KTYVQFLKGL | RLTWKTQIE | KLANIWGEH | RHKIGLLHH |
| | | GLHSIS | NRCSIS | YPYDVPDYA | ANRMGYDE | YFSLKNDM | QAISSRFGVL | CPECKASIN |
| | | ATQKSII | QTFKYY | YPYDVPDYA | VPSLHSKELA | KTYVQFLKG | LWYINRYNL | YQKIENITV |
| | | NLIQTV | CDLVIIE | YPYDVPDYA | IPLFSICRGEL | LANRMGY | TDDFSTSFVK | CQCGFKFS |
| | | NKIQIN | NRSIPT | GSGMDIEFP | RQLKNFCSD | DEVPSLHSK | YSLNWPSNF | DHLAPQAN |
| | | QIAHRL | KKIKLVS | FTDEFQKILT | AMLESFKQN | ELAIPLFSIC | YSELDEQIDK | SNALLIAQ |
| | | NISNGE | QRTFYN | TQSYPAEITN | KNTLTHDVL | RGELRQLK | AKTVQIKPFN | WLNGENTK |
| | | VLAGVL | RINALP | DKKTEVLTPS | SATFKYKFPT | NFCSDAML | KIFFNEIFDNL | LANIWGEH |
| | | SWLSKG | KYDVAL | LDSYDDAIKA | KKNPFEMNV | ESFKQNKN | LLDCQRLPTR | QAISSRFGV |
| | | TLQTVY | KRYGKR | EVLRRISFLR | ADVPIQEVES | TLTHDVLSA | EFKTNPILSH | LLWYINRY |
| | | TNEMIN | YADINY | WIKPRLKGG | YSKYNLNAM | TFKYKFPTK | VYQYFLSRY | NLTDDFSTS |
| | | GNSIVS | RTVDK | WTEKNLTPL | TDDERLTAT | KNPFEMNV | QIQPNSDVF | FVKYSLNW |
| | | LGLS | MITATR | LNDAEIDLKV | KFLDAMSLS | ADVPIQEVE | SILLSPLEASS | PSNFYSELD |
| | | (SEQ ID | PLERVEI | SAPKWRTLA | SLLSKT* | SYSGSGPA | LLSCTTDQIY | EQIDKAKTV |
| | | NO: 22) | DHTPLD | EWHKNYHK | (SEQ ID | AKKKKLDG | RLYELGFLKL | QIKPFNKIFF |
| | | | LILLDDT | SGEKVSSLIP | NO: 25) | SGKYNLNA | GVRPKLHQK | NEIFDNLLL |
| | | | LEIPLGR | KHSHKGNKN | | MTDDERLT | IASHQSVFTL | DCQRLPTR |
| | | | PYLTILI | MNTDSDFLI | | ATKFLDAM | SSIILVKLSN | EFKTNPILS |
| | | | DSYSKCI | TKAINEKYLT | | SLSSLLSKT* | MQSSQDEL | HVYQYFLSR |
| | | | VGYNLS | LNRCSISQTF | | (SEQ ID | HHYLSAW* | YQIQPNSD |
| | | | FRPPSF | KYYCDLVIIE | | NO: 26) | (SEQ ID | VFSILLSPLE |
| | | | ESIRHAF | NRSIPTKKIKL | | | NO: 27) | ASSLLSCTT |
| | | | CNACLD | VSQRTFYNRI | | | | DQIYRLYEL |
| | | | KSLITQ | NALPKYDVA | | | | GFLKLGVRP |
| | | | QYPHLQ | LKRYGKRYA | | | | KLHQKIASH |
| | | | HDWPV | DINYRTVDK | | | | QSVFTLSSII |
| | | | AGKIEN | MITATRPLER | | | | LVKLSNMQ |
| | | | LVVDN | VEIDHTPLDL | | | | SSQDELHH |
| | | | GAEFW | ILLDDTLEIPL | | | | YLSAW* |
| | | | SNSLED | GRPYLTILIDS | | | | (SEQ ID |
| | | | SLLPFAT | YSKCIVGYNL | | | | NO: 28) |
| | | | NILYNK | SFRPPSFESIR | | | | |
| | | | VGEPW | HAFCNACLD | | | | |
| | | | MKPLVE | KSLITQQYPH | | | | |
| | | | KFFDLL | LQHDWPVA | | | | |
| | | | NKGLVH | GKIENLVVD | | | | |
| | | | SLPGTT | NGAEFWSN | | | | |
| | | | RSRIEQL | SLEDSLLPFA | | | | |
| | | | KGYNPK | TNILYNKVGE | | | | |
| | | | KDAAIT | PWMKPLVE | | | | |
| | | | FSLFLEL | KFFDLLNKGL | | | | |
| | | | FHTWII | VHSLPGTTRS | | | | |
| | | | DIYHMT | RIEQLKGYNP | | | | |
| | | | SDTRET | KKDAAITFSL | | | | |
| | | | AVPYFK | FLELFHTWII | | | | |
| | | | WQEGV | DIYHMTSDT | | | | |
| | | | TALPPL | RETAVPYFK | | | | |
| | | | TYTDEE | WQEGVTAL | | | | |
| | | | AQQLRI | PPLTYTDEEA | | | | |
| | | | ELGILNT | QQLRIELGIL | | | | |
| | | | RTVRLG | NTRTVRLGGI | | | | |
| | | | GIFLHG | FLHGLRYESE | | | | |
| | | | LRYESEE | ELSEYRKIWG | | | | |
| | | | LSEYRKI | AIDKNNLTLK | | | | |
| | | | WGAID | TKTDPSDISH | | | | |
| | | | KNNLTL | IFVYLTNESR | | | | |
| | | | KTKTDP | YIKVPCITDIS | | | | |
| | | | SDISHIF | YTSGLTLFQH | | | | |
| | | | VYLTNE | QTAQKLQRT | | | | |
| | | | SRYIKVP | KTRLQIDHEK | | | | |
| | | | CITDISY | LADSRMYVE | | | | |
| | | | TSGLTLF | NRIAEEVEKI | | | | |
| | | | QHQTA | KSNKKRTAK | | | | |
| | | | QKLQRT | TTHASKIARH | | | | |
| | | | KTRLQI | QDIGSHTQK | | | | |
| | | | DHEKLA | SIQVPNEQS | | | | |
| | | | DSRMY | EIKKLNKNEH | | | | |
| | | | VENRIA | DVLNGWDE | | | | |
| | | | EEVEKIK | QHDDLEGF* | | | | |
| | | | SNKKRT | (SEQ ID | | | | |
| | | | AKTTHA | NO: 24) | | | | |
| | | | SKIARH | | | | | |
| | | | QDIGSH | | | | | |
| | | | TQKSIQ | | | | | |
| | | | VPNEQS | | | | | |
| | | | EIKKLNK | | | | | |
| | | | NEHDVL | | | | | |
| | | | NGWDE | | | | | |
| | | | QHDDL | | | | | |
| | | | EGF* | | | | | |
| | | | (SEQ ID | | | | | |
| | | | NO: 23) | | | | | |
| 4 | Tn7009 | MPISRR | YNNAEF | MPISRRNISH | MARLSTEQC | MARLSTEQ | MRFTVQTEL | MPKKKRKV |
| | | NISHSR | FIDEFVE | SRVKNLSKLS | VLLKNFKNEF | CVLLKNFKN | FKDESLESYL | GSGEQKLIS |
| | | VKNLSK | FDFNKK | NFKNPNSEK | IPHAIAETIH | EFIPHAIAET | LRLAVDNTYI | EEDLEQKLI |
| | | LSNFKN | PAKNEV | RIAESHNEFL | DDFERLREN | IHDDFERLR | DYSEFADVIG | SEEDLEQKL |
| | | PNSEKR | KLFPKD | AAHFLNYFPI | HRLGGEQLC | ENHRLGGE | RWLVDHDH | ISEEDLGSG |
| | | IAESHN | MDIFPE | VKSFQFQPL | MLIYGDEGS | QLCMLIYG | ELEGAFPCSL | RFTVQTELF |
| | | EFLAAH | KYKQEA | AFDYENQDE | GKKSIIKAYE | DEGSGKKSI | DLVNLYHAK | KDESLESYL |
| | | FLNYFPI | LAKKRYI | IHSYTSDFLV | DKCKNEEVI | IKAYEDKCK | DSSIFRVRAL | LRLAVDNTY |
| | | VKSFQF | KWVER | ELETGKFVYI | DEGKFKVPV | NEEVIDEGK | KLFETLTSFK | IDYSEFADV |
| | | QPLAFD | KLVGK | EVKEEKALYS | LFSEVKLPITV | FKVPVLFSE | PSTLLSQSLL | IGRWLVDH |
| | | YENQDE | WTEKNI | EDFKSIFEAK | NSFFTQLLID | VKLPITVNS | RTNYKFAQY | DHELEGAF |
| | | IHSYTSD | NLLLHEI | RAAARQLNK | LGEFAGAYR | FFTQLLIDL | TALKFGSSLIP | PCSLDLVNL |
| | | FLVELET | PNFGV | ELILITENQY | KAEGKNKNK | GEFAGAYR | RVMLRENKA | YHAKDSSIF |
| | | GKFVYIE | NPTPCS | NIPPRIDNIKS | DMSKQLEDI | KAEGKNKN | PIPICPQCIKE | RVRALKLFE |
| | | VKEEKA | RSIMR | LLNVGGFW | LKERLIKLETE | KDMSKQLE | SAYIRQCWH | TLTSFKPSTL |
| | | LYSEDF | WKDAY | ADDNISNLV | LIIIYKFELLLQ | DILKERLIKL | LKPYTFCHKH | LSQSLLRTN |
| | | KSIFEAK | TKGSRK | VGIVKKSETI | FDKKMRIDE | ETELIIIYKFE | NLRLLNECPK | YKFAQYTAL |
| | | RAAAR | LIALVPK | DIEGIAYHLS | LANQLKSMA | LLLQFDKK | CGDEINYIRY | KFGSSLIPR |
| | | QLNKELI | HVSKGR | QFSREEIFKAI | QELGIPLVIIG | MRIDELAN | EVIEKCICGA | VMLRENKA |
| | | LITENQ | TIAVSE | RILILKREIYF | MPCIKRLML | QLKSMAQE | DLSKMAAVH | PIPICPQCIK |
| | | YNIPPRI | HDEFIE | DLSSSELTFD | TSGWRSYIHI | LGIPLVIIG | GDIKYQKCIK | ESAYIRQC |
| | | DNIKSLL | HGISNY | SKVSSDTTQ | CRLIPYFKLS | MPCIKRLM | NLFNEIEGDK | WHLKPYTF |
| | | NVGGF | LTELRLS | GSGPKKKRK | NELEKAFYVK | LTSGWRSYI | SSEIGKLLWF | CHKHNLRLL |
| | | WADDN | INECYK | VGSGYPYDV | VIKGLSNRA | HICRLIPYFK | SKYKNIELDD | NECPKCGD |
| | | ISNLVV | KYETQL | PDYAYPYDV | QKLFSFAPKL | LSNELEKAF | TELLNEFYDY | EINYIRYEVI |
| | | GIVKKS | RTNTDL | PDYAYPYDV | EDKSISYPLF | YVKVIKGLS | FEFWPATYL | EKCICGADL |
| | | ETIDIEG | EPVSYN | PDYAGSGYN | AVSSGCFRTI | NRAQKLFSF | SELEQFELGG | SKMAAVH |
| | | IAYHLS | TFKLRID | NAEFFIDEFV | RNYTNKAVL | APKLEDKSI | INKQIRPFNQ | GDIKYQKCI |
| | | QFSREEI | KLPKYD | EFDFNKKPA | LAVNEGAEE | SYPLFAVSS | TPVNDIWKE | KNLFNEIEG |
| | | FKAIRILI | VKCARE | KNEVKLFPK | LTIEHFSKVF | GCFRTIRNY | QIALSKLASP | DKSSEIGKL |
| | | LKREIYF | GKAAA | DMDIFPEKY | ERDNEYPLL | TNKAVLLA | FKQNNEVLK | LWFSKYKNI |
| | | DLSSSEL | DIDENN | KQEALAKKR | GKVDSTNDD | VNEGAEEL | VLSEYFVDLV | ELDDTELLN |
| | | TFDSKV | YDEHCP | YIKWVERKL | SMKKLNESIK | TIEHFSKVF | YRYPKSETLN | EFYDYFEF |
| | | SSDTTQ | PKRLYE | VGKWTEKNI | DERDKRDSI | ERDNEYPLL | PADTLLTKLE | WPATYLSEL |
| | | (SEQ ID | QVEIDH | NLLLHEIPNF | NPFKISVDKL | GKVDSTND | ASILLRTPLE | EQFELGGIN |
| | | NO: 29) | TVLTVIL | GVNPTPCSR | MVNEVIDYA | DSMKKLNE | QVNRLLNEN | KQIRPFNQT |
| | | | LDSEYLF | SIMRWKDAY | TYKYDEESAS | SIKDERDKR | YLHRAIKPKK | PVNDIWKE |
| | | | PIGRPTL | TKGSRKLIAL | EVKFDTRFA | DSINPFKISV | HEIIEPFKPLL | QIALSKLAS |
| | | | TVLIDKL | VPKHVSKGR | DKISINDLLR | DKLMVNEV | YLRQVIELME | PFKQNNEV |
| | | | SHCICG | TIAVSEHDEF | K* (SEQ ID | IDYAGSGPA | VRGINQAYS | LKVLSEYFV |
| | | | FYVSYE | IEHGISNYLT | NO: 32) | AKKKKLDG | NLYTTTW | DLVYRYPKS |
| | | | PPSYNS | ELRLSINECY | | SGTYKYDEE | (SEQ ID | ETLNPADTL |
| | | | ARQAIL | KKYETQLRT | | SASEVKFDT | NO: 34 | LTKLEASILL |
| | | | HSIKPK | NTDLEPVSY | | RFADKISIN | | RTPLEQVN |
| | | | DYIKNL | NTFKLRIDKL | | DLLRK* | | RLLNENYLH |
| | | | YPSIKNE | PKYDVKCAR | | (SEQ ID | | RAIKPKKHE |
| | | | WNCHG | EGKAAADID | | NO: 33) | | IIEPFKPLLYL |
| | | | KIENLIV | FNNYDEHCP | | | | RQVIELME |
| | | | DNGAEF | PKRLYEQVEI | | | | VRGINQAY |
| | | | WSTNLE | DHTVLTVILL | | | | SNLYTTTW |
| | | | VACEN | DSEYLFPIGR | | | | (SEQ ID |
| | | | WMNIQ | PTLTVLIDKLS | | | | NO: 35) |
| | | | FNPVGK | HCICGFYVSY | | | | |
| | | | PWKKA | EPPSYNSAR | | | | |
| | | | FVERFIG | QAILHSIKPK | | | | |
| | | | TTCREF | DYIKNLYPSIK | | | | |
| | | | TARFKG | NEWNCHGK | | | | |
| | | | KTFSNIL | IENLIVDNGA | | | | |
| | | | EKMKY | EFWSTNLEV | | | | |
| | | | DPKKDA | ACENWMNI | | | | |
| | | | VMRFD | QFNPVGKP | | | | |
| | | | LFLELFH | WKKAFVERF | | | | |
| | | | KWIIDD | IGTTCREFTA | | | | |
| | | | YHQRA | RFKGKTFSNI | | | | |
| | | | DSRFKYI | LEKMKYDPK | | | | |
| | | | PNELW | KDAVMRFDL | | | | |
| | | | QKNYLK | FLELFHKWII | | | | |
| | | | SPVLKL | DDYHQRADS | | | | |
| | | | DQAEEE | RFKYIPNEL | | | | |
| | | | KLENDF | WQKNYLKSP | | | | |
| | | | LCTEWR | VLKLDQAEE | | | | |
| | | | EWRKG | EKLENDFLCT | | | | |
| | | | GIHIFNL | EWREWRKG | | | | |
| | | | RYDSEY | GIHIFNLRYD | | | | |
| | | | LSKVRK | SEYLSKVRKQ | | | | |
| | | | QYVKEG | YVKEGNDKK | | | | |
| | | | NDKKQ | QKILVKYSPE | | | | |
| | | | KILVKYS | NINTIRIYIED | | | | |
| | | | PENINTI | LGKYIEVPCV | | | | |
| | | | RIYIEDL | DSVGYTKGL | | | | |
| | | | GKYIEV | SLFNHQVNL | | | | |
| | | | PCVDSV | RVHRTYIKSK | | | | |
| | | | GYTKGL | IDVVSLAEVR | | | | |
| | | | SLFNHQ | KYVNDRVEE | | | | |
| | | | VNLRVH | EEEFVEKGRK | | | | |
| | | | RTYIKSK | KNLSANKAR | | | | |
| | | | IDVVSL | SRYKSINSKN | | | | |
| | | | AEVRKY | SISKKDNKFE | | | | |
| | | | VNDRV | DIEKSEDASP | | | | |
| | | | EEEEEF | EDWNNFAE | | | | |
| | | | VEKGRK | GLEGF* | | | | |
| | | | KNLSAN | (SEQ ID | | | | |
| | | | KARSRY | NO: 31) | | | | |
| | | | KSINSK | | | | | |
| | | | NSISKK | | | | | |
| | | | DNKFED | | | | | |
| | | | IEKSEDA | | | | | |
| | | | SPEDW | | | | | |
| | | | NNFAE | | | | | |
| | | | GLEGF | | | | | |
| | | | (SEQ ID | | | | | |
| | | | NO: 30) | | | | | |
| 5 | Tn7011 | MYRRKL | MFNDG | MYRRKLKHS | MLTDKQKAK | MLTDKQKA | MHFLVQTKL | MPKKKRKV |
| | | KHSRVK | LFDDEF | RVKNLHKFA | LNEFRDVFIE | KLNEFRDVF | YPDEALESYL | GSGEQKLIS |
| | | NLHKFA | NQPLPK | SQKNKSTCL | YPIITTVEND | IEYPIITTVF | LRLARDNSY | EEDLEQKLI |
| | | SQKNKS | VETKLP | VESSLEFDAC | FDRLRLGKGL | NDFDRLRL | DGYSELADIL | SEEDLEQKL |
| | | TCLVESS | QNYAK | FHFEFSPSIA | AGEKPCMLL | GKGLAGEK | WQWLVEQD | ISEEDLGSG |
| | | LEFDAC | DLQALP | AFEAQPLGY | NGDTGTGKT | PCMLLNGD | HDLEGALPLE | HFLVQTKLY |
| | | FHFEFS | EKIKNTT | EYEFDNRICR | ALIKQYKERH | TGTGKTALI | LGKVDVYHA | PDEALESYL |
| | | PSIAAFE | FAKLKYI | YTPDFLLTHT | LPQFINGVM | KQYKERHL | RQASSFRIRA | LRLARDNSY |
| | | AQPLGY | QWLEA | DGTQKFIEVK | NHPVLVSRIP | PQFINGVM | LKLVAQLAD | DGYSELADI |
| | | EYEFDN | NIQGG | PQSKIADEDF | SNPTLESTLA | NHPVLVSRI | VNAGNILAL | LWQWLVE |
| | | RICRYTP | WTQKN | RARFIEKQTI | ELLKDLGQV | PSNPTLEST | AWRRSNFKF | QDHDLEGA |
| | | DFLLTH | LEPLLKV | AKQDGRDLI | GSTERKLRIN | LAELLKDLG | GNLVAVSRN | LPLELGKVD |
| | | TDGTQK | MPEVE | LVTDKQIRVY | GTRLTTSLIK | QVGSTERK | EQTIPLELLRT | VYHARQAS |
| | | FIEVKP | GEKKPS | PTLNNLKLLH | CLKTCGTELII | LRINGTRLT | DNIPVCIECL | SFRIRALKLV |
| | | QSKIAD | WRTAA | RYSGFQSLTE | IDEFQELIEH | TSLIKCLKTC | FESSYVPFH | AQLADVNA |
| | | EDFRAR | RWYSA | LQASVLELVK | NQGKKRREI | GTELIIIDEF | WHLKPYKTC | GNILALAW |
| | | FIEKQTI | YTNADK | QYGSIKVGQ | ANRLKYINDE | QELIEHNQ | HKHKSQLTT | RRSNFKFG |
| | | AKQDG | NIMALI | LVNFLKVTA | AGVSIVLVG | GKKRREIAN | HCKECHNLI | NLVAVSRN |
| | | RDLILVT | PSHQKK | GELLATVLRL | MPWAEKIA | RLKYINDEA | DYRASEEFLE | EQTIPLELLR |
| | | DKQIRV | GNRER | LSLGQLFADL | DEPQWSSRL | GVSIVLVG | CSCGCKLTN | TDNIPVCIE |
| | | YPTLNN | DTATDK | TTNEISIETAI | LVRRQLPYFK | MPWAEKIA | SEQLNDADF | CLFESSYVP |
| | | LKLLHR | FFEKALE | WSNNVGSG | LSENPKHFV | DEPQWSSR | KIAFALASSN | FHWHLKPY |
| | | YSGFQS | RYLVKE | PKKKRKVGS | QLIIGLANR | LLVRRQLPY | SHKIVGLISW | KTCHKHKS |
| | | LTELQA | KPSVAS | GYPYDVPDY | MPFTEKPKL | FKLSENPKH | FAKVKQLDV | QLTTHCKEC |
| | | SVLELV | AYKYYA | AYPYDVPDY | SEQATVFALF | FVQLIIGLA | SDADFNRTF | HNLIDYRAS |
| | | KQYGSI | DLVIIEN | AYPYDVPDY | SLSKGCFRTL | NRMPFTEK | VDYFSTWPE | EEFLECSCG |
| | | KVGQLV | DSVVGS | AGSGFNDGL | KYFLDDAVLY | PKLSEQATV | SLTTELDLLT | CKLTNSEQL |
| | | NFLKVT | VLKPLTY | FDDEFNQPL | ALMDNAKTL | FALFSLSKG | NNARLKQLN | NDADFKIAF |
| | | AGELLA | KAFKNR | PKVETKLPQ | TTKHLVKAF | CFRTLKYFL | PFNKTKENS | ALASSNSHK |
| | | TVLRLLS | IDNLPQ | NYAKDLQAL | GVLFPDVPN | DDAVLYAL | VYGNVIRDG | IVGLISWFA |
| | | LGQLFA | YDVMV | PEKIKNTTFA | LFTLPVAEIT | MDNAKTLT | QIAATSNRK | KVKQLDVS |
| | | DLTTNEI | SRYGKR | KLKYIQWLE | ASEVERYSLY | TKHLVKAF | NKVLDELIKY | DADFNRTF |
| | | SIETAIW | LADIAF | ANIQGGWT | KLESAQDED | GVLFPDVP | FVELVDSNP | VDYFSTWP |
| | | SNNV* | NKVEG | QKNLEPLLKV | PFIATKFTDQ | NLFTLPVAE | KTKHPNIADL | ESLTTELDLL |
| | | (SEQ ID | HTRPTR | MPEVEGEKK | MPISQLLRK* | ITASEVERY | LLCTFDTAVL | TNNARLKQ |
| | | NO: 36) | VLEKVEI | PSWRTAAR | (SEQ ID | SGSGPAAK | LNTTTEQVY | LNPFNKTKF |
| | | | DHTPLD | WYSAYTNA | NO: 39) | KKKLDGSGL | RLHQEGFLN | NSVYGNV! |
| | | | LILLDDE | DKNIMALIPS | | YKLESAQDE | CAYPQKKHE | RDGQIAAT |
| | | | LHIPLGR | HQKKGNRER | | DPFIATKFT | QLRADSHVF | SNRKNKVL |
| | | | PTLTML | DTATDKFFE | | DQMPISQL | YLRQVIELQQ | DELIKYFVEL |
| | | | VDVYSH | KALERYLVKE | | LRK* (SEQ | AFAAETPQT | VDSNPKTK |
| | | | CIVGFYF | KPSVASAYKY | | ID NO: 40) | KKQFIAPW* | HPNIADLLL |
| | | | SFSEPSY | YADLVIIEND | | | (SEQ ID | CTFDTAVLL |
| | | | DAVRR | SVVGSVLKPL | | | NO: 41) | NTTTEQVY |
| | | | AMLNA | TYKAFKNRID | | | | RLHQEGFL |
| | | | MKPKS | NLPQYDVM | | | | NCAYPQKK |
| | | | DVAKLY | VSRYGKRLA | | | | HEQLRADS |
| | | | PDTINE | DIAFNKVEG | | | | HVFYLRQVI |
| | | | WKCAG | HTRPTRVLEK | | | | ELQQAFAA |
| | | | KIETLVV | VEIDHTPLDL | | | | ETPQTKKQ |
| | | | DNGAEF | ILLDDELHIPL | | | | FIAPW* |
| | | | WSNSLE | GRPTLTMLV | | | | (SEQ ID |
| | | | LACEEIG | DVYSHCIVGF | | | | NO: 42) |
| | | | INTQYN | YFSFSEPSYD | | | | |
| | | | PVAKP | AVRRAMLN | | | | |
| | | | WLKPFV | AMKPKSDVA | | | | |
| | | | ERMFG | KLYPDTINE | | | | |
| | | | TINTELL | WKCAGKIET | | | | |
| | | | DPVPGK | LVVDNGAEF | | | | |
| | | | TFSNILQ | WSNSLELAC | | | | |
| | | | KHEYNP | EEIGINTQYN | | | | |
| | | | KKDAIM | PVAKPWLKP | | | | |
| | | | RFTTFM | FVERMFGTI | | | | |
| | | | QLFHK | NTELLDPVP | | | | |
| | | | WVVDV | GKTFSNILQK | | | | |
| | | | YHQDA | HEYNPKKDA | | | | |
| | | | DSRFKYI | IMRFTTFMQ | | | | |
| | | | PSQLW | LFHKWVVD | | | | |
| | | | EQGFNT | VYHQDADSR | | | | |
| | | | LPPTVLS | FKYIPSQLWE | | | | |
| | | | NADLQ | QGFNTLPPT | | | | |
| | | | QLDVVL | VLSNADLQQ | | | | |
| | | | SISNHR | LDVVLSISNH | | | | |
| | | | VLRKGG | RVLRKGGIRL | | | | |
| | | | IRLENLS | ENLSYDSTEL | | | | |
| | | | YDSTEL | ANYRKQFSH | | | | |
| | | | ANYRK | KVSQEVLIKL | | | | |
| | | | QFSHKV | NPDDISYIYV | | | | |
| | | | SQEVLIK | YLDKLEHYIK | | | | |
| | | | LNPDDI | VPCIDPNGY | | | | |
| | | | SYIYVYL | TQNLSLNQH | | | | |
| | | | DKLEHYI | KINIRIHRDFI | | | | |
| | | | KVPCID | SGSIDNVGL | | | | |
| | | | PNGYT | AKARMFIHN | | | | |
| | | | QNLSLN | KIQNEFEELK | | | | |
| | | | QHKINI | NAPKHSKVK | | | | |
| | | | RIHRDFI | GGKALAKHQ | | | | |
| | | | SGSIDN | NVSSDSQKSI | | | | |
| | | | VGLAKA | AQSEPLEPKK | | | | |
| | | | RMFIHN | VTPKEQPTD | | | | |
| | | | KIQNEF | SWDDFISDL | | | | |
| | | | EELKNA | DGF* (SEQ | | | | |
| | | | PKHSKV | ID NO: 38) | | | | |
| | | | KGGKAL | | | | | |
| | | | AKHQN | | | | | |
| | | | VSSDSQ | | | | | |
| | | | KSIAQS | | | | | |
| | | | EPLEPK | | | | | |
| | | | KVTPKE | | | | | |
| | | | QPTDS | | | | | |
| | | | WDDFIS | | | | | |
| | | | DLDGF* | | | | | |
| | | | (SEQ ID | | | | | |
| | | | NO: 37) | | | | | |
| 6 | Tn7014 | MYVRN | MSFGPF | SFGPFEDEFG | MTSLQPTNN | MTSLQPTN | MDTDIEVYS | MPKKKRKV |
| | | LRKPSA | EDEFGSI | SITNDVQQQ | DVDVLLAEF | NDVDVLLA | DESLESFLLRL | GSGEQKLIS |
| | | NKNVYK | TNDVQ | YEASPEAKLS | HQSFVVYPD | EFHQSFVV | SKFQGYERF | EEDLEQKLI |
| | | FVSVKN | QQYEAS | RLKYSPLETT | VEKVFEGLD | YPDVEKVFE | AHFAEDIWQ | SEEDLEQKL |
| | | GCNIM | PEAKLS | KVIERDLSSF | WIVRRSQFG | GLDWIVRR | TTLLQHEAIP | ISEEDLGSG |
| | | CESSLEY | RLKYSPL | PEEQKLKALE | KFAPSMLITG | SQFGKFAPS | GAFPFELSRI | DTDIEVYSD |
| | | DCCYYL | ETTKVIE | RYKLISLIAKEI | GTGAGKTSV | MLITGGTG | NIYKAQTTS | ESLESFLLRL |
| | | EYSDDV | RDLSSF | NGGWTPKN | VEIYLNNHFS | AGKTSVVEI | QMRVRVLID | SKFQGYERF |
| | | VRYQSQ | PEEQKL | LIPLIDKHIET | SSEVLITRVR | YLNNHFSSS | LEKRLKFNDF | AHFAEDIW |
| | | PKGYRF | KALERY | LNIPKPSDRT | PSFVETLIWA | EVLITRVRP | GVLRLSLAHS | QTTLLQHE |
| | | PYQGKE | KLISLIA | VKRWYKAFC | IEKLNVPYNS | SFVETLIWA | KASFSPDYKA | AIPGAFPFE |
| | | HPYTPD | KEINGG | ESDGDIKSLV | RSKRSEIGLQ | IEKLNVPYN | VNRYGADYP | LSRINIYKA |
| | | FLVHKK | WTPKN | DSHHLKGNR | DYFINSVKKS | SRSKRSEIG | QAFLRKNFT | QTTSQMRV |
| | | DGTSYL | LIPLIDK | QPRIEDDEPF | KLKLLVIEEA | LQDYFINSV | PVCPKCLDE | RVLIDLEKR |
| | | LEVKPLS | HIETLNI | FIEAVERFLD | QELFECASPK | KKSKLKLLVI | AAYIRQLWH | LKFNDFGVL |
| | | KTFSSEF | PKPSDR | AVRPSYSKAY | ERQKIRDRLK | EEAQELFEC | FIPYQVCHK | RLSLAHSKA |
| | | QDVFR | TVKRW | QVYCDRIEIE | MISDECRLPI | ASPKERQKI | HHSQLAQRC | SFSPDYKAV |
| | | QKQIM | YKAFCE | NSTIVSGKIA | VFIGIPTAKLI | RDRLKMIS | PECGKLLNY | NRYGADYP |
| | | ASELGA | SDGDIK | KVSYEAFKKR | LEDSQWDR | DECRLPIVFI | QSSELIENCE | QAFLRKNF |
| | | PLLLVT | SLVDSH | LKKLPPYTVA | RIMVKRDLP | GIPTAKLILE | CGFSLLNGES | TPVCPKCLD |
| | | DRQIRN | HLKGNR | LKRHGKYYA | YIRITNEESLD | DSQWDRRI | EKESCSTLFV | EAAYIRQL |
| | | DVHLN | QPRIED | DKLFNYYEA | VYIALLEGLE | MVKRDLPY | AQWLAGEK | WHFIPYQV |
| | | NLKLVH | DEPFFIE | VKMPTRILER | KTLPISVAPE | IRITNEESLD | PVESGLMSQ | CHKHHSQL |
| | | RYSGCI | AVERFL | VEIDHTPLDL | LTDMDMA | VYIALLEGLE | ELTQSSRFGF | AQRCPECG |
| | | GNSSHL | DAVRPS | ILLDDELLVPL | MRLLAASRG | KTLPISVAP | LLWYINRYG | KLLNYQSSE |
| | | ESVWS | YSKAYQ | GRAYLTLLVD | MLGLIKELVG | ELTDMDM | ELDDISFDGF | LIENCECGF |
| | | AVNQSS | VYCDRI | VFSGCIIGFH | YAFELALLEG | AMRLLAAS | VECCKSWPN | SLLNGESEK |
| | | SICIKAL | EIENSTI | LGFKAPSYTA | KRQITQNEF | RGMLGLIKE | KLNTDLDSIV | ESCSTLFVA |
| | | SAILNLT | VSGKIA | VSKAIIHSVK | VQAFKSIFGP | LVGYAFELA | QKADIVRIQP | QWLAGEKP |
| | | IGEVFA | KVSYEA | SKEYVNELPI | DISNPFEIELD | LLEGKRQIT | WNKIYFSEV | VESGLMSQ |
| | | SVLRLIG | FKKRLK | GLSNQWICH | KLLIPQIIEYE | QNEFVQAF | FGDLLKECRS | ELTQSSRFG |
| | | LGKAKT | KLPPYT | GKIENLVVD | GYLLDSDSG | KSIFGPDIS | LPSRDLSKNP | FLLWYINRY |
| | | KLDVLL | VALKRH | NGAEFWSKS | DIKFTHQIFE | NPFEIELDK | VLKNVVLYFR | GELDDISFD |
| | | DENSLIS | GKYYAD | LDQACIEAGI | DIPLTELLR* | LLIPQIIEYE | ALITNNPKVK | GFVECCKS |
| | | VA* | KLFNYY | NIIYNKVRKP | (SEQ ID | GSGPAAKK | SANIGDVLLS | WPNKLNTD |
| | | (SEQ ID | EAVKM | WLKPFVERK | NO: 46) | KKLDGSGG | PLEASTLLSC | LDSIVQKAD |
| | | NO: 43) | PTRILER | FGELIQGIVG | | YLLDSDSGD | TTDEIYRLYQ | IVRIQPWN |
| | | | VEIDHT | WVPGRTFSN | | IKFTHQIFE | FGQLKAQHT | KIYFSEVFG |
| | | | PLDLILL | VLEKEDYDP | | DIPLTELLR* | PKLESKIENH | DLLKECRSL |
| | | | DDELLV | QKDAVMRF | | (SEQ ID | HSVFTLRSIIE | PSRDLSKNP |
| | | | PLGRAY | SVFVEELHR | | NO: 47) | LKLSSMCSET | VLKNVVLYF |
| | | | LTLLVD | WIIDVHNAS | | | DGLNHYLPE | RALITNNPK |
| | | | VFSGCII | ADSRHTRIP | | | W* (SEQ ID | VKSANIGD |
| | | | GFHLGF | NYHWQKSE | | | NO: 48) | VLLSPLEAS |
| | | | KAPSYT | EVLPPPALTE | | | | TLLSCTTDEI |
| | | | AVSKAII | RDEIQFRVI | | | | YRLYQFGQ |
| | | | HSVKSK | MGMVHKG | | | | LKAQHTPKL |
| | | | EYVNEL | ALTSKGIKFK | | | | QSKIENHHS |
| | | | PIGLSN | HLMYDNVAL | | | | VFTLRSIIEL |
| | | | QWICH | EHYRKQYPQ | | | | KLSSMCSET |
| | | | GKIENL | SKDSRIKTVKI | | | | DGLNHYLP |
| | | | VVDNG | DPDDLSRIFV | | | | EW* (SEQ |
| | | | AEFWSK | FLEEKKGYIE | | | | ID NO: 49) |
| | | | SLDQAC | VPCKYDPLG | | | | |
| | | | IEAGINII | YTKKLSLCEH | | | | |
| | | | YNKVRK | RTVKVHRD | | | | |
| | | | PWLKPF | FIKGQVDSLS | | | | |
| | | | VERKFG | LAKARQALH | | | | |
| | | | ELIQGIV | ERIKQEHENL | | | | |
| | | | GWVPG | RQMSLPHRA | | | | |
| | | | RTFSNV | KKAKNGKK | | | | |
| | | | LEKEDY | MAELAGVN | | | | |
| | | | DPQKD | SDSPKSITTD | | | | |
| | | | AVMRF | YPIEDTIQLH | | | | |
| | | | SVFVEE | ESTPVDDLQ | | | | |
| | | | LHRWII | SLWNKRRAL | | | | |
| | | | DVHNA | RKSGK* | | | | |
| | | | SADSRH | (SEQ ID | | | | |
| | | | TRIPNY | NO: 45) | | | | |
| | | | HWQKS | | | | | |
| | | | EEVLPP | | | | | |
| | | | PALTER | | | | | |
| | | | DEIQFR | | | | | |
| | | | VIMGM | | | | | |
| | | | VHKGAL | | | | | |
| | | | TSKGIKF | | | | | |
| | | | KHLMY | | | | | |
| | | | DNVALE | | | | | |
| | | | HYRKQY | | | | | |
| | | | PQSKDS | | | | | |
| | | | RIKTVKI | | | | | |
| | | | DPDDLS | | | | | |
| | | | RIFVFLE | | | | | |
| | | | EKKGYIE | | | | | |
| | | | VPCKYD | | | | | |
| | | | PLGYTK | | | | | |
| | | | KLSLCE | | | | | |
| | | | HLRTVK | | | | | |
| | | | VHRDFI | | | | | |
| | | | KGQVD | | | | | |
| | | | SLSLAK | | | | | |
| | | | ARQALH | | | | | |
| | | | ERIKQE | | | | | |
| | | | HENLRQ | | | | | |
| | | | MSLPHR | | | | | |
| | | | AKKAKN | | | | | |
| | | | GKKMA | | | | | |
| | | | ELAGVN | | | | | |
| | | | SDSPKSI | | | | | |
| | | | TTDYPIE | | | | | |
| | | | DTIQLH | | | | | |
| | | | ESTPVD | | | | | |
| | | | DLQSL | | | | | |
| | | | WNKRR | | | | | |
| | | | ALRKSG | | | | | |
| | | | K* (SEQ | | | | | |
| | | | ID | | | | | |
| | | | NO: 44) | | | | | |
| 7 | Tn7015 | MYIRNL | MIEFKD | MYIRNLRKP | MNTLTAHQ | MNTLTAH | MAFLFSPKA | MPKKKRKV |
| | | RKPSPN | EFTESTS | SPNKNVFKF | MEQLGRFN | QMEQLGRF | RSFSDESLES | GSGEQKLIS |
| | | KNVFKF | VKKPDT | ASAKVSETI | DCFVMHPQ | NDCFVMH | YLLRVVAENF | EEDLEQKLI |
| | | ASAKVS | PGQYIK | MCESTLEFD | AKVIFNDFD | PQAKVIFN | FDSYQQLSL | SEEDLEQKL |
| | | ETIMCE | LDDAEIL | ACFHHEYNE | DLRLNRNFQ | DFDDLRLN | AIREELHELD | ISEEDLGSG |
| | | STLEFD | KRDLDT | TIETFGSQPK | SDQQCMLLT | RNFQSDQQ | FEAHGAFPV | AFLFSPKAR |
| | | ACFHHE | FPDFLK | GFYYCFEGK | GDTGVGKSH | CMLLTGDT | ELKRLNVYH | SFSDESLES |
| | | YNETIET | EKAFDK | RLPYTPDALL | LINNYKKRVL | GVGKSHLIN | AKHNSHFR | YLLRVVAEN |
| | | FGSQPK | YKLISFIE | HYIDGTTKFH | ASQTYSRTS | NYKKRVLAS | MRALGLLES | FFDSYQQLS |
| | | GFYYCF | QENSG | EYKPYSKTFD | MPVLVTRISS | QTYSRTSM | LLDLPPHELQ | LAIREELHEL |
| | | EGKRLP | GWTQK | PIFRAKFVAK | HKGLDATLR | PVLVTRISS | KLALLRSNKR | DFEAHGAF |
| | | YTPDAL | KLDPILD | KEAAQALGT | QMLTDLESF | HKGLDATL | FVGGMSAV | PVELKRLNV |
| | | LHYIDG | KLFEGN | ELILVTDKQI | GSQQRKGQ | RQMLTDLE | HRNGVDIPL | YHAKHNSH |
| | | TTKFHE | RDKRPN | RVNPILNNLK | NYKIDLKTQL | SFGSQQRK | SFIRCADEDG | FRMRALGL |
| | | YKPYSK | WRTVV | LLHRYSGIYG | VKNLVRANV | GQNYKIDLK | IESLPICPQCL | LESLLDLPP |
| | | TFDPIFR | RWRKS | VTDIQRELLQ | ELLIFNEFQEL | TQLVKNLV | KEEPYIRQA | HELQKLALL |
| | | AKFVAK | YIDSNG | LIRHSGKIQL | IEFKTPKERQ | RANVELLIF | WHIKPIEVC | RSNKRFVG |
| | | KEAAQA | DLASLV | DDVADEYEL | TIANELKFISE | NEFQELIEF | AKHECELIHH | GMSAVHR |
| | | LGTELIL | VKRHK | SVGETRSFLY | EARVPIVLVG | KTPKERQTI | CPDCQQPIS | NGVDIPLSF |
| | | VTDKQI | MGNRK | SLINKGLLEA | MPWTEQIA | ANELKFISE | YIENESITHCS | IRCADEDGI |
| | | RVNPIL | KRVEGD | DLTQDDLSC | EEPQWSSRLI | EARVPIVLV | CGFEFATASS | ESLPICPQC |
| | | NNLKLL | EVFFER | NPFVWCNA | RRRKLEYFSL | GMPWTEQ | EKADSQAVV | LKEEPYIRQ |
| | | HRYSGI | ALSRFL | GSGPKKKRK | QKDSKYYRQ | IAEEPQWS | LSRSLFDGDA | AWHIKPIEV |
| | | YGVTDI | DAKRPK | VGSGYPYDV | YLIGLAKHM | SRLIRRRKLE | LSNNPLLFM | CAKHECELI |
| | | QRELLQ | VTTAYQ | PDYAYPYDV | PFDEPPKIED | YFSLQKDSK | GTSVTHRFA | HHCPDCQ |
| | | LIRHSG | YYKDAI | PDYAYPYDV | KHIAIPLFAA | YYRQYLIGL | ALLWYLKRH | QPISYIENES |
| | | KIQLDD | TIENETI | PDYAGSGIEF | CRGESRVLN | AKHMPFDE | VQNIECKLDE | ITHCSCGFE |
| | | VADEYE | VDGEIPI | KDEFTESTSV | HLLSETLKLV | PPKIEDKHI | SVNYFEAWP | FATASSEKA |
| | | LSVGET | ISYTAFN | KKPDTPGQYI | MVNGDRSL | AIPLFAACR | ENFYQELDEL | DSQAVVLS |
| | | RSFLYSL | QRIKSLP | KLDDAEILKR | DIRHLAQTY | GESRVLNH | LAGAELKLID | RSLFDGDAL |
| | | INKGLLE | PYPIAV | DLDTFPDFLK | RKLYESQESE | LLSETLKLV | LFNRTSLSFIF | SNNPLLFM |
| | | ADLTQD | ARHGKF | EKAFDKYKLI | AASVFFNPFL | MVNGDRSL | GELILQSQCL | GTSVTHRF |
| | | DLSCNP | KADQW | SFIEQENSGG | EPLDKVLISE | DIRHLAQTY | LPEDKTPHFI | AALLWYLK |
| | | FVWCN | FAYCSS | WTQKKLDPI | VVKPSRYNP | RKLYESQES | DMGLMEYL | RHVQNIEC |
| | | A* (SEQ | HIPPTRI | LDKLFEGNR | NAMTPDEM | EAASVFFNP | GKLVESHPKS | KLDESVNYF |
| | | ID | LERVEID | DKRPNWRT | LIKREFSAPST | FLEPLDKVLI | KKPNVADM | EAWPENFY |
| | | NO: 50) | HTPLDLI | VVRWRKSYI | LAQLLSK* | SEVVKPSGS | LVSVTETAVL | QELDELLAG |
| | | | LLDDEL | DSNGDLASL | (SEQ ID | GPAAKKKK | LSTSHEQVYR | AELKLIDLF |
| | | | QLPLGR | VVKRHKMG | NO: 53) | LDGSGRYN | LYQDGVLTA | NRTSLSFIF |
| | | | PYLTLIV | NRKKRVEGD | | PNAMTPDE | GFKQKIRTRI | GELILQSQC |
| | | | DVFSNC | EVFFERALSR | | MLIKREFSA | DPHIGVFYLR | LLPEDKTPH |
| | | | VLGFHL | FLDAKRPKV | | PSTLAQLLS | QVIEYKTSFG | FIDMGLME |
| | | | SYKAPS | TTAYQYYKD | | K* (SEQ ID | NDKQGMYL | YLGKLVESH |
| | | | YVSAAK | AITIENETIVD | | NO: 54) | SAW* (SEQ | PKSKKPNV |
| | | | AIVHAIK | GEIPIISYTAF | | | ID NO: 55) | ADMLVSVT |
| | | | PKTLGIV | NQRIKSLPPY | | | | ETAVLLSTS |
| | | | GIELQN | PIAVARHGK | | | | HEQVYRLY |
| | | | DWPCY | FKADQWFA | | | | QDGVLTAG |
| | | | GKFETL | YCSSHIPPTRI | | | | FKQKIRTRI |
| | | | VVDNG | LERVEIDHTP | | | | DPHIGVFYL |
| | | | AEFWSK | LDLILLDDEL | | | | RQVIEYKTS |
| | | | SLDHAC | QLPLGRPYLT | | | | FGNDKQG |
| | | | KEAGINI | LIVDVFSNCV | | | | MYLSAW* |
| | | | QYNPV | LGFHLSYKAP | | | | (SEQ ID |
| | | | RKPWLK | SYVSAAKAIV | | | | NO: 56) |
| | | | PFVERF | HAIKPKTLGI | | | | |
| | | | FGMIN | VGIELQNDW | | | | |
| | | | QYFLTE | PCYGKFETLV | | | | |
| | | | LPGKTF | VDNGAEFW | | | | |
| | | | SNILEKE | SKSLDHACKE | | | | |
| | | | DYKPEK | AGINIQYNP | | | | |
| | | | DAIMRF | VRKPWLKPF | | | | |
| | | | SVFVEE | VERFFGMIN | | | | |
| | | | FHRWIV | QYFLTELPGK | | | | |
| | | | DIYHQD | TFSNILEKED | | | | |
| | | | SDSRDT | YKPEKDAIM | | | | |
| | | | RIPIKQ | RFSVFVEEFH | | | | |
| | | | WQHGF | RWIVDIYHQ | | | | |
| | | | DVYPPL | DSDSRDTRIP | | | | |
| | | | QMSVE | IKQWQHGF | | | | |
| | | | DEKRFN | DVYPPLQMS | | | | |
| | | | VLMGIT | VEDEKRFNV | | | | |
| | | | DERTLT | LMGITDERTL | | | | |
| | | | RNGFKF | TRNGFKFEEL | | | | |
| | | | EELMYD | MYDSTALAD | | | | |
| | | | STALAD | YRKHYPQTK | | | | |
| | | | YRKHYP | DTIKKLIKIDP | | | | |
| | | | QTKDTI | DDLSNIHVYL | | | | |
| | | | KKLIKID | EELEGYLKVP | | | | |
| | | | PDDLSN | CTDTTGYAN | | | | |
| | | | IHVYLEE | GLSLHEHKVI | | | | |
| | | | LEGYLK | KKINREIIRES | | | | |
| | | | VPCTDT | KDNLGLAKA | | | | |
| | | | TGYAN | RMAIHARVQ | | | | |
| | | | GLSLHE | QEQELFNES | | | | |
| | | | HKVIKKI | KTKAKISAVK | | | | |
| | | | NREIIRE | KQAQLADIS | | | | |
| | | | SKDNLG | NTGQGTIRL | | | | |
| | | | LAKAR | ENSDTLSDIT | | | | |
| | | | MAIHA | NKPESNISDI | | | | |
| | | | RVQQE | LDNWDDNIE | | | | |
| | | | QELFNE | GFE* (SEQ | | | | |
| | | | SKTKAKI | ID NO: 52) | | | | |
| | | | SAVKKQ | | | | | |
| | | | AQLADI | | | | | |
| | | | SNTGQ | | | | | |
| | | | GTIRLE | | | | | |
| | | | NSDTLS | | | | | |
| | | | DITNKP | | | | | |
| | | | ESNISDI | | | | | |
| | | | LDNWD | | | | | |
| | | | DNIEGF | | | | | |
| | | | E* (SEQ | | | | | |
| | | | ID | | | | | |
| | | | NO: 51) | | | | | |
| 8 | Tn7016 | MYIRNL | MTDFF | MYIRNLRKP | MNALTEIQIE | MNALTEIQI | MAFLFSPKA | MPKKKRKV |
| | | RKPSPN | NEFDES | SPNKNVFKF | KLRNFSDCIV | EKLRNFSDC | RAFSDESLES | GSGEQKLIS |
| | | KNVFKF | LVPLKP | ASTKVSSVV | MHPQIKTIF | IVMHPQIKT | YLLRVVSENF | EEDLEQKLI |
| | | ASTKVS | QTPTQY | MCESSLEFD | NDFDELRLN | IFNDFDELR | FDSYEGLSLA | SEEDLEQKL |
| | | SVVMC | VKLDDA | ACFHHEYND | RKFQSDQQC | LNRKFQSD | IREELHELDF | ISEEDLGSG |
| | | ESSLEFD | NLIQRD | LIESFGSQPE | MLLIGDTGV | QQCMLLIG | EAHGAFPVD | AFLFSPKAR |
| | | ACFHHE | LDTFSD | GFKYEFMGK | GKSHTINHY | DTGVGKSH | LKRLNVYHA | AFSDESLES |
| | | YNDLIES | TFKNQA | SLPYTPDALIS | KKRVLATQN | TINHYKKRV | KHNSHFRM | YLLRVVSEN |
| | | FGSQPE | LQRYKLI | YTDKTQKYH | YSRNTMPVL | LATQNYSR | RALGLLETLL | FFDSYEGLS |
| | | GFKYEF | STIDKKL | EYKPYSKIAS | VSRISRGKGL | NTMPVLVS | DLPRYELQKL | LAIREELHEL |
| | | MGKSLP | SRGWT | PLFRAEFAAK | DATLVQMLA | RISRGKGLD | ALLKSDIKFN | DFEAHGAF |
| | | YTPDALI | QRNLDP | RAASLKLGID | DLELFGSSQI | ATLVQMLA | SSVALYNNG | PVDLKRLN |
| | | SYTDKT | ILDELFK | LVLVTDRQIR | KKRGYKTDL | DLELFGSSQ | VDIPLRFIRH | VYHAKHNS |
| | | QKYHEY | GGDVV | VNPILNNLKL | TKKLVESLIK | IKKRGYKTD | HAEEAVDSIP | HFRMRALG |
| | | KPYSKIA | RPNWR | LHRYSGVYGI | AQVELLIINE | LTKKLVESLI | VCSQCLAEE | LLETLLDLPR |
| | | SPLFRA | TVARW | SGIQKELLSFI | FQELIEFKSV | KAQVELLII | AYIKQSWHI | YELQKLALL |
| | | EFAAKR | RKKYIES | HKSGVIKLN | QERQQIANG | NEFQELIEF | KWVNACTK | KSDIKFNSS |
| | | AASLKL | NGDIAS | DISSQVGIPI | LKFISEEAKV | KSVQERQQ | HQCALLHNC | VALYNNGV |
| | | GIDLVL | LADKNH | GETRSFLFGL | PIVLVGMPW | IANGLKFISE | PECYAPINYI | DIPLRFIRH |
| | | VTDRQI | KMGNR | MHKGLVKA | AAKIAEEPQ | EAKVPIVLV | ENESITHCSC | HAEEAVDSI |
| | | RVNPIL | TNRIKG | DLGCDDLTN | WASRLVRKR | GMPWAAK | GFELSCASTS | PVCSQCLA |
| | | NNLKLL | DDKFFD | NPTLWATPG | KLEYFSLKND | IAEEPQWA | PVNTLSIEHL | EEAYIKQS |
| | | HRYSGV | KALERF | SGPKKKRKV | SKYFRQYLM | SRLVRKRKL | NKLLDKGER | WHIKWVN |
| | | YGISGIQ | LDAKRP | GSGYPYDVP | GLAKKMPFD | EYFSLKNDS | NDSNPLFNN | ACTKHQCA |
| | | KELLSFI | TIATAY | DYAYPYDVP | VPPKLESKNT | KYFRQYLM | MTLTERFAA | LLHNCPECY |
| | | HKSGVI | QYYKDL | DYAYPYDVP | TIALFAACRG | GLAKKMPF | LLWYQERYS | APINYIENE |
| | | KLNDISS | IVIENESI | DYAGSGTDF | ENRALKHLLL | DVPPKLESK | QTDNFCLND | SITHCSCGF |
| | | QVGIPI | VEGKIPI | FNEFDESLVP | EALKLALSCN | NTTIALFAA | AVNYFSKWP | ELSCASTSP |
| | | GETRSF | ISYNAF | LKPQTPTQY | EYLENKHFIT | CRGENRAL | AVFNTELDEL | VNTLSIEHL |
| | | LFGLMH | NKRIKAI | VKLDDANLI | AYDKFDFFN | KHLLLEALK | SKNAEMKLI | NKLLDKGE |
| | | KGLVKA | PPYAVA | QRDLDTFSD | DKEKLKSKN | LALSCNEYL | DLFNKTEFKF | RNDSNPLF |
| | | DLGCD | VARHG | TFKNQALQR | PFKQDIKDIEI | ENKHFITAY | IFGDAILACP | NNMTLTER |
| | | DLTNNP | KFKADQ | YKLISTIDKKL | YEVIKNSSYN | DKFDFFND | STQKQSESH | FAALLWYQ |
| | | TLWATP | WFAYC | SRGWTQRN | PNALDPED | KEKLKSKNP | FIYRALLDYL | ERYSQTDN |
| | | * (SEQ | AAHVPP | LDPILDELFK | MLTDRVFAI | FKQDIKDIEI | VTLVESNPKT | FCLNDAVN |
| | | ID | TRILERV | GGDVVRPN | VK* (SEQ ID | YEVIKNSGS | KKPNAADLL | YFSKWPAV |
| | | NO: 57) | EIDHTPL | WRTVARWR | NO: 60) | GPAAKKKK | VSVLEAATLL | FNTELDELS |
| | | | DLILLDD | KKYIESNGDI | | LDGSGSYN | GTSVEQVYR | KNAEMKLI |
| | | | ELLIPIG | ASLADKNHK | | PNALDPED | LYQNGILQT | DLFNKTEFK |
| | | | RPYLTLL | MGNRTNRIK | | MLTDRVFA | AFRHKMNQ | FIFGDAILA |
| | | | IDVFSG | GDDKFFDKA | | IVK* (SEQ | RINPYKGAFF | CPSTQKQS |
| | | | CVLGFH | LERFLDAKRP | | ID NO: 61) | LRHVIEYKTS | ESHFIYRALL |
| | | | LSYKSPS | TIATAYQYYK | | | FGNDKARM | DYLVTLVES |
| | | | YVSAAK | DLIVIENESIV | | | YLSAW* | NPKTKKPN |
| | | | AITHAIK | EGKIPIISYNA | | | (SEQ ID | AADLLVSVL |
| | | | PKSLDA | FNKRIKAIPP | | | NO: 62) | EAATLLGTS |
| | | | LNIELQ | YAVAVARHG | | | | VEQVYRLY |
| | | | NDWPC | KFKADQWF | | | | QNGILQTA |
| | | | FGKFEN | AYCAAHVPP | | | | FRHKMNQ |
| | | | LVVDN | TRILERVEID | | | | RINPYKGAF |
| | | | GAEFW | HTPLDLILLD | | | | FLRHVIEYK |
| | | | SKNLEH | DELLIPIGRPY | | | | TSFGNDKA |
| | | | ACQSA | LTLLIDVFSG | | | | RMYLSAW* |
| | | | GINIQY | CVLGFHLSYK | | | | (SEQ ID |
| | | | NPVRKP | SPSYVSAAKA | | | | NO: 63) |
| | | | WLKPFI | ITHAIKPKSL | | | | |
| | | | ERFFGV | DALNIELQN | | | | |
| | | | MNEYFL | DWPCFGKFE | | | | |
| | | | PELPGK | NLVVDNGAE | | | | |
| | | | TFSNILE | FWSKNLEHA | | | | |
| | | | KEEYKP | CQSAGINIQY | | | | |
| | | | EKDAIM | NPVRKPWLK | | | | |
| | | | RFSTFV | PFIERFFGV | | | | |
| | | | EEFHR | MNEYFLPEL | | | | |
| | | | WIADVY | PGKTFSNILE | | | | |
| | | | HQDSN | KEEYKPEKD | | | | |
| | | | SRETRIP | AIMRFSTFVE | | | | |
| | | | IKRWQ | EFHRWIADV | | | | |
| | | | QGFDA | YHQDSNSRE | | | | |
| | | | YPPLTM | TRIPIKRWQ | | | | |
| | | | NEEEET | QGFDAYPPL | | | | |
| | | | RFSML | TMNEEEETR | | | | |
| | | | MRISDS | FSMLMRISD | | | | |
| | | | RTLTRN | SRTLTRNGFK | | | | |
| | | | GFKYQE | YQELMYDST | | | | |
| | | | LMYDST | ALADYRKHY | | | | |
| | | | ALADYR | PQTKETVKKL | | | | |
| | | | KHYPQT | IKVDPDDISKI | | | | |
| | | | KETVKK | YVYLEELESYL | | | | |
| | | | LIKVDP | EVPCTDPTG | | | | |
| | | | DDISKIY | YTDGLSIYEH | | | | |
| | | | VYLEELE | KTIKKINREVI | | | | |
| | | | SYLEVP | RESKDSLGLA | | | | |
| | | | CTDPTG | KARMAIHER | | | | |
| | | | YTDGLSI | VKQEQEVFIE | | | | |
| | | | YEHKTIK | SKTKAKITAV | | | | |
| | | | KINREVI | KKQAQIADV | | | | |
| | | | RESKDS | SNTGTSTIKV | | | | |
| | | | LGLAKA | SEESAAPVQ | | | | |
| | | | RMAIHE | KHISNDNSD | | | | |
| | | | RVKQE | DWDDDLEA | | | | |
| | | | QEVFIES | FE* (SEQ ID | | | | |
| | | | KTKAKIT | NO: 59) | | | | |
| | | | AVKKQ | | | | | |
| | | | AQIADV | | | | | |
| | | | SNTGTS | | | | | |
| | | | TIKVSEE | | | | | |
| | | | SAAPVQ | | | | | |
| | | | KHISND | | | | | |
| | | | NSDDW | | | | | |
| | | | DDDLEA | | | | | |
| | | | FE* | | | | | |
| | | | (SEQ ID | | | | | |
| | | | NO: 58) | | | | | |
| 10 | V.para_ | MFDQT | MVASEL | MFDQTKKSS | MNITPEQRA | MNITPEQR | MNSNIQLYR | MPKKKRKV |
| | UCM-V493 | KKSSHV | DNFVGF | HVHNICKFM | QLAAYENCFI | AQLAAYEN | DESLESFLLRL | GSGEQKLIS |
| | AHI99014 | HNICKF | FDEME | SLKNDAVVR | EYPEITEIYSIF | CFIEYPEITEI | SQEQGYGRF | EEDLEQKLI |
| | | MSLKN | ASRSEA | TLSILEFDFCF | DQLRFNQSL | YSIFDQLRF | SHFAEELWY | SEEDLEQKL |
| | | DAVVRT | QMESQI | HLEYNPDVE | GGEPESFLLT | NQSLGGEP | QTLDDSSGL | ISEEDLGSG |
| | | LSILEFD | PVELFQ | KYLSQPHGY | GEAGSGKTA | ESFLLTGEA | SGAFPLELSR | NSNIQLYRD |
| | | FCFHLE | SDTDHS | HYQFNNRKC | LIDNYLSRFE | GSGKTALID | VNVYHAQTT | ESLESFLLRL |
| | | YNPDVE | SSFDSLP | RYTPDFLVFD | VSANSWSQ | NYLSRFEVS | SQMRVRVFI | SQEQGYGR |
| | | KYLSQP | EKTQKE | RQERSSFIEIK | QTILSTRIPSR | ANSWSQQ | YLENQLKLSN | FSHFAEEL |
| | | HGYHY | VLRRLKI | HSSQILKPDF | VNEQNTLTQ | TILSTRIPSR | FRVLRLALTH | WYQTLDDS |
| | | QFNNR | IQYVEV | RARFAEKQR | FLIDLDVKSG | VNEQNTLT | SKSHFSPDLK | SGLSGAFPL |
| | | KCRYTP | RLKGG | VAREEHDKR | GRGVRRRNE | QFLIDLDVK | AVHRLGVDY | ELSRVNVY |
| | | DFLVFD | WTEKN | LILITEKQIRIN | IALAEAVVA | SGGRGVRR | PYAFLRKRFT | HAQTTSQ |
| | | RQERSS | LDPILN | PIFNNLKLLH | QLKRKSVELII | RNEIALAEA | PVCPSCLSEA | MRVRVFIYL |
| | | FIEIKHS | MVENA | RYSGLHSVTK | VNEVQELIEF | VVAQLKRK | PYIRQHWHL | ENQLKLSNF |
| | | SQILKP | LELPRPS | VQKTVLGYI | STAQERQVI | SVELIIVNEV | IPHQVCEKH | RVLRLALTH |
| | | DFRARF | WRTLAS | QRKQRVKLY | ANTFKYISEE | QELIEFSTA | GCDLIHRCPE | SKSHFSPDL |
| | | AEKQRV | WKKDY | EVSEYLGLSE | ARVSFVLVG | QERQVIAN | CDALLEYQS | KAVHRLGV |
| | | AREEHD | YESGKK | HETLTSALC | MPYASVLAQ | TFKYISEEAR | VESITQCECG | DYPYAFLRK |
| | | KRLILITE | WLSLIP | WLSSGKVKT | EPQWDSRLS | VSFVLVGM | FHLLEALPKP | RFTPVCPSC |
| | | KQIRINP | KHTQK | DFKSADFSL | WRRNLDYFK | PYASVLAQE | ASESDLLVAR | LSEAPYIRQ |
| | | IFNNLKL | GNRTA | NSYVWCGS | LFKSKINEKN | PQWDSRLS | WLTGNHLEV | HWHLIPHQ |
| | | LHRYSG | HTDSQF | GPKKKRKVG | TARSYEIDTL | WRRNLDYF | VGPMGKAM | VCEKHGCD |
| | | LHSVTK | IIDEAIA | SGYPYDVPD | QKKHFAKFV | KLFKSKINE | SISERYGLLL | IHRCPECD |
| | | VQKTVL | KKYLTR | YAYPYDVPD | AGLASRMGY | KNTARSYEI | WYVNRYGSL | ALLEYQSVE |
| | | GYIQRK | ERLSVA | YAYPYDVPD | DNPPKLTKN | DTLQKKHF | EEFSLGEFVQ | SITQCECGF |
| | | QRVKLY | ETYRYY | YAGSGVASE | DTLYPLFVM | AKFVAGLA | YCAMWPKR | HLLEALPKP |
| | | EVSEYL | KSRVIKT | LDNFVGFFD | CRGECRRLK | SRMGYDNP | LHQDLDML | ASESDLLVA |
| | | GLSEHE | NQTIVE | EMEASRSEA | HFLSDAMIM | PKLTKNDTL | AKKAELVRIK | RWLTGNHL |
| | | TLTSALC | GKIELIS | QMESQIPVE | SFKESTDTID | YPLFVMCR | KWKQTFFYE | EVVGPMG |
| | | WLSSGK | QRAFYD | LFQSDTDHS | KETLSRAFAF | GECRRLKHF | AFGTLLKECR | KAMSISERY |
| | | VKTDFK | RVNGLP | SSFDSLPEKT | KFPHMANPF | LSDAMIMS | YLPSRQLSKN | GLLLWYVN |
| | | SADFSL | AYDVAV | QKEVLRRLKII | ACSLSEIKLS | FKESTDTID | IVLAELLRYF | RYGSLEEFS |
| | | NSYVW | ARYGKR | QYVEVRLKG | QIDTNSMYN | KETLSRAFA | NRLVADHPS | LGEFVQYC |
| | | C (SEQ | YADRHF | GWTEKNLDP | TTAIATEDRIL | FKFPHMAN | SVKGNIVDIL | AMWPKRL |
| | | ID | RSVGQ | ILNMVENAL | APRFTDDFPL | PFACSLSEIK | LSPLEASTLLS | HQDLDML |
| | | NO: 64) | QVSATK | ELPRPSWRT | SMLLSKSGV | LSQIDTNSG | CTTDEIYRLY | AKKAELVRI |
| | | | PMEYVE | LASWKKDYY | KI (SEQ ID | SGPAAKKK | EYGEIKAAVR | KKWKQTFF |
| | | | IDHTPIP | ESGKKWLSLI | NO: 67) | KLDGSGMY | PQMHVKIAS | YEAFGTLLK |
| | | | VILIDDE | PKHTQKGNR | | NTTAIATED | HESVFTLRSV | ECRYLPSRQ |
| | | | LDVPLG | TAHTDSQFII | | RILAPRFTD | VETKLARMC | LSKNIVLAE |
| | | | RPYLTM | DEAIAKKYLT | | DFPLSMLLS | SESDGLSVYL | LLRYFNRLV |
| | | | LYDRFS | RERLSVAETY | | KSGVKI* | PEW* (SEQ | ADHPSSVK |
| | | | KCIVGLS | RYYKSRVIKT | | (SEQ ID | ID NO: 69) | GNIVDILLS |
| | | | VNFREP | NQTIVEGKIE | | NO: 68) | | PLEASTLLS |
| | | | SFDSVR | LISQRAFYDR | | | | CTTDEIYRL |
| | | | KALLNA | VNGLPAYDV | | | | YEYGEIKAA |
| | | | LLNKN | AVARYGKRY | | | | VRPQMHV |
| | | | WVKDK | ADRHFRSVG | | | | KIASHESVF |
| | | | YPSVKN | QQVSATKP | | | | TLRSVVETK |
| | | | DWPCC | MEYVEIDHT | | | | LARMCSES |
| | | | GKIDYL | PIPVILIDDEL | | | | DGLSVYLPE |
| | | | VVDNG | DVPLGRPYLT | | | | W* (SEQ ID |
| | | | AEFWSK | MLYDRFSKCI | | | | NO: 70) |
| | | | SLEDSLK | VGLSVNFRE | | | | |
| | | | PLVLDI | PSFDSVRKAL | | | | |
| | | | QYSQA | LNALLNKNW | | | | |
| | | | AKPWR | VKDKYPSVK | | | | |
| | | | KSGIEKL | NDWPCCGKI | | | | |
| | | | FDQLNK | DYLVVDNGA | | | | |
| | | | GLTNSL | EFWSKSLED | | | | |
| | | | PGKTFT | SLKPLVLDIQ | | | | |
| | | | NPTQLE | YSQAAKPW | | | | |
| | | | DYDPKK | RKSGIEKLFD | | | | |
| | | | ESVVRV | QLNKGLTNS | | | | |
| | | | SVFLELL | LPGKTFTNPT | | | | |
| | | | HKWVI | QLEDYDPKK | | | | |
| | | | DYYHM | ESVVRVSVFL | | | | |
| | | | SPDARE | ELLHKWVID | | | | |
| | | | RDVPYH | YYHMSPDAR | | | | |
| | | | KWHES | ERDVPYHK | | | | |
| | | | RWLPN | WHESRWLP | | | | |
| | | | TYEDEE | NTYEDEEKS | | | | |
| | | | KSRLKIE | RLKIELGLLR | | | | |
| | | | LGLLRH | HRTIGLAGIR | | | | |
| | | | RTIGLA | LHNLRYQSD | | | | |
| | | | GIRLHN | ELIEYRKYCS | | | | |
| | | | LRYQSD | VKYERKLFVK | | | | |
| | | | ELIEYRK | TKTDPSDISSI | | | | |
| | | | YCSVKY | YVYLEFENRY | | | | |
| | | | ERKLFV | IRVPAVDNS | | | | |
| | | | KTKTDP | GYTQGLSLFE | | | | |
| | | | SDISSIY | HERIQRVRRL | | | | |
| | | | VYLEFE | NTKRMVDEE | | | | |
| | | | NRYIRV | ALADTYLYM | | | | |
| | | | PAVDNS | ESRIEAETER | | | | |
| | | | GYTQGL | LRNYGDRKR | | | | |
| | | | SLFEHE | SQPKIGNTSK | | | | |
| | | | RIQRVR | LAKFRDVGT | | | | |
| | | | RLNTKR | TGPSSIITTSV | | | | |
| | | | MVDEE | NEPLTNSYD | | | | |
| | | | ALADTY | GIVTDLDDE | | | | |
| | | | LYMESR | DFDEIEGY* | | | | |
| | | | IEAETER | (SEQ ID | | | | |
| | | | LRNYGD | NO: 66) | | | | |
| | | | RKRSQP | | | | | |
| | | | KIGNTS | | | | | |
| | | | KLAKFR | | | | | |
| | | | DVGTTG | | | | | |
| | | | PSSIITTS | | | | | |
| | | | VNEPLT | | | | | |
| | | | NSYDGI | | | | | |
| | | | VTDLDD | | | | | |
| | | | EDFDEIE | | | | | |
| | | | GY (SEQ | | | | | |
| | | | ID | | | | | |
| | | | NO: 65) | | | | | |
| 11 | Alii- | MKKRKL | MASED | MKKRKLTKS | MSEFGEKLK | MSEFGEKL | MSMLLIRTK | MPKKKRKV |
| | glaci- | TKSAVN | TFSGLF | AVNNIHRFA | LVRELFIAGP | KLVRELFIA | PFLDESLESY | GSGEQKLIS |
| | ecola | NIHRFA | DLVVEE | SFKMDDFIE | YLESLMCEID | GPYLESLM | LLRLSIHNGY | EEDLEQKLI |
| | sp. M165 | SFKMD | NCSMP | VESTLEFDAC | ECKEDSKLG | CEIDECKED | NKFQSFWA | SEEDLEQKL |
| | | DFIEVES | DGLQPT | FHFEYSAKVL | GEAQCMFIT | SKLGGEAQ | GVRSHLNES | ISEEDLGSG |
| | | TLEFDA | EPATFR | EFESQPIGFE | GNTGSGKTT | CMFITGNT | TRGIDSALPS | SMLLIRTKP |
| | | CFHFEY | ALSVFT | YELDGKIRSY | LIRKYMENYP | GSGKTTLIR | ELSKINICHA | FLDESLESYL |
| | | SAKVLE | TIQRDQ | TPDYLARLET | RKELADRTKI | KYMENYPR | NVSSAKRLD | LRLSIHNGY |
| | | FESQPI | AIHRLN | LPSTFYEVKL | PVFFTSLPEN | KELADRTKI | ALRLVSQLTN | NKFQSFWA |
| | | GFEYEL | LIKYLLK | YKKTLSEIFKS | ATPVRASQK | PVFFTSLPE | HEPLPLLSLA | GVRSHLNE |
| | | DGKIRS | AGVRSF | EFKAKQVAA | MLTDLGDPF | NATPVRAS | LFRGGQLFS | STRGIDSAL |
| | | YTPDYL | TEKTITP | EALGGRLELI | SCVSSDLEEL | QKMLTDLG | RKRTSVENN | PSELSKINIC |
| | | ARLETL | LLPDLV | TENNIRVYPL | RIKLICLLVSC | DPFSCVSSD | GVTIPFRFLR | HANVSSAK |
| | | PSTFYE | TEFGND | LDNLKILHRY | GVELIIIDEFQ | LEELRIKLICL | TKGIPICPACI | RLDALRLVS |
| | | VKLYKK | VPSWR | HSAENDLSD | HLIERKNNK | LVSCGVELII | KENVYIRQH | QLTNHEPL |
| | | TLSEIFK | TLARW | QQYQAITILG | VLHRAADW | IDEFQHLIE | WHFSLFEAC | PLLSLALFR |
| | | SEFKAK | WSLFKA | RVERLSILDLI | LKTIIIDSNIP | RKNNKVLH | PEHSVLLRN | GGQLFSRK |
| | | QVAAE | SDFDIV | HRMGQNYR | VVLVGMPYS | RAADWLKT | HCDCGEEIN | RTSVFNNG |
| | | ALGGRL | ALVPQI | EIFPDILSLVA | SVILDVNSQL | IIIDSNIPVV | YLSSHEIAQC | VTIPFRFLRT |
| | | ELITEN | TKGNSN | LDLLKLDMN | NDRMLFKRR | LVGMPYSS | AKCGSNLAD | KGIPICPACI |
| | | NIRVYP | FKADPL | MPISTDSIIW | LPPFRVEEES | VILDVNSQL | LEATVSSAP | KENVYIRQ |
| | | LLDNLKI | LEPLIAE | CSKGSGPKK | ERKVYLQFLK | NDRMLFKR | QREIAHWLS | HWHFSLFE |
| | | LHRYHS | AIGRIM | KRKVGSGYP | VFDLALPFPD | RLPPFRVEE | GRLVEGLPA | ACPEHSVLL |
| | | AENDLS | SAERPN | YDVPDYAYP | SSSLQTREVA | ESERKVYLQ | VIQSHSWGI | RNHCDCGE |
| | | DQQYQ | LAEGHR | YDVPDYAYP | LRLYSHSKGN | FLKVFDLAL | CLWWQETF | EINYLSSHEI |
| | | AITILGR | FLETLVL | YDVPDYAGS | LRKLRELLNQ | PFPDSSSLQ | NDGKDIDSE | AQCAKCGS |
| | | VERLSIL | RYNKG | GASEDTFSG | ASRDALLMS | TREVALRLY | QLHLFLAQW | NLADLEAT |
| | | DLIHRM | NDTQL | LFDLVVEENC | ANCITSEHFK | SHSKGNLR | PDSLRSYLNC | VSSAPQREI |
| | | GQNYR | QCISSE | SMPDGLQPT | SAIDKINGNY | KLRELLNQA | KLAHSKEYAL | AHWLSGRL |
| | | EIFPDIL | ALRLRV | EPATFRALSV | SDTVNPFNV | SRDALLMS | KPFNQLSFK | VEGLPAVIQ |
| | | SLVALD | GKITPFE | FTTIQRDQAI | SHINDVAIDE | ANCITSEHF | DVFGLLLIQA | SHSWGICL |
| | | LLKLDM | EIKARK | HRLNLIKYLL | PDLDIGWED | KSAIDKING | SRLPSTNLSE | WWQETFN |
| | | NMPIST | GLTAAN | KAGVRSFTE | FKNKPGEILV | NYSDTVNP | NIVLKEIVRYL | DGKDIDSE |
| | | DSIIWC | NEFRAI | KTITPLLPDL | GKSSRQFTV | FNVSHIND | EEHVFEPECL | QLHLFLAQ |
| | | SK* | GQKIKT | VTEFGNDVP | GDIFATR* | VAIDEPDLD | LSDLKLNSIE | WPDSLRSY |
| | | (SEQ ID | TRILERV | SWRTLARW | (SEQ ID | IGSGPAAKK | AAIILGTSVE | LNCKLAHSK |
| | | NO: 71) | EVDHTR | WSLFKASDF | NO: 74) | KKLDGSGG | QIAVLVDQG | EYALKPFN |
| | | | LDLFVID | DIVALVPQIT | | WEDFKNKP | ELQTKSRMK | QLSFKDVF |
| | | | DIYFIP | KGNSNFKAD | | GEILVGKSS | ANSVLNAN | GLLLIQASR |
| | | | MGRP | PLLEPLIAEAI | | RQFTVGDIF | WRVLSLGDV | LPSTNLSEN |
| | | | WLTMLI | GRIMSAERP | | ATR* (SEQ | FCLWLAKFQ | IVLKEIVRYL |
| | | | DSFSLS | NLAEGHRFL | | ID NO: 75) | TDNSHSNVFI | EEHVFEPEC |
| | | | VVGFYL | ETLVLRYNKG | | | SRW* (SEQ | LLSDLKLNSI |
| | | | GFEPPS | NDTQLQCISS | | | ID NO: 76) | EAAIILGTSV |
| | | | FVSVSH | EALRLRVGKI | | | | EQIAVLVD |
| | | | ALKNAIL | TPFEEIKARK | | | | QGELQTKS |
| | | | PKSYVK | GLTAANNEF | | | | RMKANSVL |
| | | | ENYPQV | RAIGQKIKTT | | | | NANWRVLS |
| | | | NNEWI | RILERVEVDH | | | | LGDVFCLW |
| | | | CSGLIEL | TRLDLFVIDD | | | | LAKFQTDN |
| | | | LVTDNG | IYFIPMGRP | | | | SHSNVFISR |
| | | | REFDDK | WLTMLIDSF | | | | W* (SEQ ID |
| | | | DFKVAC | SLSVVGFYLG | | | | NO: 77) |
| | | | AELGM | FEPPSFVSVS | | | | |
| | | | HVGKN | HALKNAILPK | | | | |
| | | | PTKKPY | SYVKENYPQ | | | | |
| | | | LKASVE | VNNEWICSG | | | | |
| | | | RFFGTV | LIELLVTDNG | | | | |
| | | | NSRLLA | REFDDKDFK | | | | |
| | | | SPPGKT | VACAELGM | | | | |
| | | | FPNIFER | HVGKNPTKK | | | | |
| | | | DDYDPE | PYLKASVERF | | | | |
| | | | KNAVIS | FGTVNSRLLA | | | | |
| | | | LSKINLLI | SPPGKTFPNI | | | | |
| | | | HKWIID | FERDDYDPE | | | | |
| | | | DYQQD | KNAVISLSKI | | | | |
| | | | PNARW | NLLIHKWIID | | | | |
| | | | TNMPN | DYQQDPNA | | | | |
| | | | LSWSVA | RWTNMPNL | | | | |
| | | | AQSFPP | SWSVAAQSF | | | | |
| | | | ATYNGS | PPATYNGSID | | | | |
| | | | IDELDFK | ELDFKLGRRF | | | | |
| | | | LGRRFE | EPKLRKEGIT | | | | |
| | | | PKLRKE | KDKLRYHSD | | | | |
| | | | GITKDK | RLASYRGRY | | | | |
| | | | LRYHSD | GDHRVIAKQ | | | | |
| | | | RLASYR | DPNNLGRIV | | | | |
| | | | GRYGD | VLDNDKKEY | | | | |
| | | | HRVIAK | FFVPAVDFD | | | | |
| | | | QDPNN | YANGLTLW | | | | |
| | | | LGRIVVL | QHNLHRKYT | | | | |
| | | | DNDKKE | KEFIKANYNH | | | | |
| | | | YFFVPA | QDVVQARSE | | | | |
| | | | VDFDYA | IIDIVEGCMA | | | | |
| | | | NGLTL | EMATGKRKK | | | | |
| | | | WQHNL | ISVTNRVRA | | | | |
| | | | HRKYTK | GRYLEADRR | | | | |
| | | | EFIKAN | RELPSPNTSE | | | | |
| | | | YNHQD | TVERNEKKEI | | | | |
| | | | VVQARS | PFSEESWDE | | | | |
| | | | EIIDIVE | DVDISEWTS | | | | |
| | | | GCMAE | SQVRK* | | | | |
| | | | MATGK | (SEQ ID | | | | |
| | | | RKKISVT | NO: 73) | | | | |
| | | | NRVRA | | | | | |
| | | | GRYLEA | | | | | |
| | | | DRRREL | | | | | |
| | | | PSPNTS | | | | | |
| | | | ETVERN | | | | | |
| | | | EKKEIPF | | | | | |
| | | | SEESWD | | | | | |
| | | | EDVDIS | | | | | |
| | | | EWTSS | | | | | |
| | | | QVRK* | | | | | |
| | | | (SEQ ID | | | | | |
| | | | NO: 72) | | | | | |
| 12 | Qceano- | MYNRN | MFEDEY | MYNRNLRKP | MPKLTDAQK | MPKLTDAQ | MPRLPAHIQ | MPKKKRKV |
| | spir- | LRKPSP | SPEYID | SPVKNVYKF | ANIRQFKDSF | KANIRQFK | IYSDESLESYL | GSGEQKLIS |
| | illum | VKNVYK | NLDGGF | ASRKNHSTI | CLYYSIKKLLS | DSFCLYYSIK | LRLCQANYF | EEDLEQKLI |
| | linum | FASRKN | IEHNEG | MCESSLEFD | DLETVFESSEI | KLLSDLETV | DSFYDFALEL | SEEDLEQKL |
| | ATCC | HSTIMC | EEDTYD | ACFHLEYSDK | GGEPLSMLIT | FESSEIGGE | KHLLWEQES | ISEEDLGSG |
| | 11336 | ESSLEFD | LDCFPK | VVNFASQPT | GDTGSGKSS | PLSMLITGD | GAAGGLPTE | PRLPAHIQI |
| | | ACFHLE | EQQQIA | GIEYFDNAN | TINHFIKSKIS | TGSGKSSTI | LAAINIYHAQ | YSDESLESY |
| | | YSDKVV | VAKTKFI | KKRRYTPDFS | PQTGRAPILS | NHFIKSKISP | QDSGRRSQA | LLRLCQANY |
| | | NFASQP | NIRKKL | VSYQDGTSN | TRVPSRATA | QTGRAPILS | FLVEKMLEL | FDSFYDFAL |
| | | TGIEYF | KDKGW | LIEVKPAKKL | EETTKQMLI | TRVPSRATA | KPFTLLDITFK | ELKHLLWE |
| | | DNANK | TKENV | LSPDFQNDF | DLGVFGSSV | EETTKQMLI | HGTSVDLYQ | QESGAAGG |
| | | KRRYTP | MPIVDA | SQKLNAYKEI | SSRKSSDQN | DLGVFGSS | RATVSYQNH | LPTELAAINI |
| | | DFSVSY | LYDASL | GETLILVTEN | LTNRLISAVK | VSSRKSSDQ | IIPRHYLRQN | YHAQQDSG |
| | | QDGTS | PFKSPSL | QIRSEPTLTN | DSGIKLIIINE | NLTNRLISA | SIPICPVCLQ | RRSQALFLV |
| | | NLIEVKP | SSVQR | YKILHRYASF | FQELVEFKKP | VKDSGIKLIII | GEQPYIRYL | EKMLELKPF |
| | | AKKLLS | WHRSLS | LGDSELQAEI | KDQQVISNR | NEFQELVEF | WHLEPVKAC | TLLDITFKH |
| | | PDFQN | QNQDN | KKRLHETKNL | LKVISESTEV | KKPKDQQV | VEHNCKLVE | GTSVDLYQ |
| | | DFSQKL | PAVLVS | SVARLASLLN | PLIFVGMPW | ISNRLKVISE | CCPRCNETL | RATVSYQN |
| | | NAYKEI | KHHRK | LEEQNLIPVC | SDEIRQDPQ | STEVPLIFV | NYMESELITH | HIIPRHYLR |
| | | GETLILV | GNRNS | AMMLAKGY | WSSRLATRS | GMPWSDEI | CFCGFDLRK | QNSIPICPV |
| | | TENQIR | KVGDD | LTADLQASKF | HNIEYFSIIKK | RQDPQWS | CEQEPADAK | CLQGEQPYI |
| | | SEPTLT | KYFDLA | TELTLTPFED | PRQFRDFMK | SRLATRSHN | SYWQLNPEA | RYLWHLEP |
| | | NYKILH | LERFLK | GSGPKKKRK | ALKSHIPIQR | IEYFSIIKKPR | FSAFGDCSFS | VKACVEHN |
| | | RYASFL | ATRPTA | VGSGYPYDV | SDDMDNME | QFRDFMKA | EKLAVLSLLE | CKLVECCPR |
| | | GDSELQ | MSAYRY | PDYAYPYDV | EDLRIFAATC | LKSHIPIQRS | QLASDKNQE | CNETLNYM |
| | | AEIKKRL | YESQML | PDYAYPYDV | GEQRQIKAL | DDMDNME | VLLREGIDFF | ESELITHCFC |
| | | HETKNL | IDIENGK | PDYAGSGFE | MTEVYRLCLI | EDLRIFAAT | SRLLEERISE | GFDLRKCE |
| | | SVARLA | YEGRPIS | DEYSPEYIDN | QEQPISLKIY | CGEQRQIK | QLTLATKPLS | QEPADAKS |
| | | SLLNLEE | QTAFYK | LDGGFIEHN | DEAFRNLYP | ALMTEVYR | KLSFRTLSAG | YWQLNPEA |
| | | QNLIPV | RLAKLSS | EGEEDTYDL | TANDQPFKG | LCLIQEQPIS | LIDELSKVSN | FSAFGDCSF |
| | | CAMML | YEVTAK | DCFPKEQQQ | KLEQVNFREI | LKIYDEAFR | LPQGLISGVI | SEKLAVLSLL |
| | | AKGYLT | RYGKYK | IAVAKTKFIL | EMSSRYIRG | NLYPTAND | KAILIKALDTP | EQLASDKN |
| | | ADLQAS | ADMKF | NIRKKLKDKG | DSMYPAHIE | QPFKGKLE | KTSLGCLGDS | QEVLLREGI |
| | | KFTELTL | GYKGGP | WTKENVMP | PAKLSEFYSL | QVNFREIE | LLSPRECAFL | DFFSRLLEE |
| | | TPFED | LKLERPL | IVDALYDASL | SELLSKS* | MSSGSGPA | LQSSVNDIYR | RISEQLTLA |
| | | (SEQ ID | QRVEID | PFKSPSLSSV | (SEQ ID | AKKKKLDG | LYETGVLSPA | TKPLSKLSF |
| | | NO: 78) | HTPLDLI | QRWHRSLS | NO: 81) | SGRYIRGDS | IRLPSKQTIQ | RTLSAGLID |
| | | | LLDDET | QNQDNPAV | | MYPAHIEP | SYQTIFRLQD | ELSKVSNLP |
| | | | LHPLGR | LVSKHHRKG | | AKLSEFYSLS | IAGFTLSCSSF | QGLISGVIK |
| | | | PYLTILK | NRNSKVGD | | ELLSKS* | MAVTSSR* | AILIKALDTP |
| | | | DSLSKCI | DKYFDLALER | | (SEQ ID | (SEQ ID | KTSLGCLGD |
| | | | IGYHLSF | FLKATRPTA | | NO: 82) | NO: 83) | SLLSPRECA |
| | | | QAPSYA | MSAYRYYES | | | | FLLQSSVND |
| | | | SASKAIC | QMLIDIENG | | | | IYRLYETGV |
| | | | HAMLP | KYEGRPISQT | | | | LSPAIRLPSK |
| | | | KKIKGP | AFYKRLAKLS | | | | QTIQSYQTI |
| | | | DGKPS | SYEVTAKRY | | | | FRLQDIAGF |
| | | | WECHG | GKYKADMKF | | | | TLSCSSFMA |
| | | | KIETLVA | GYKGGPLKL | | | | VTSSR* |
| | | | DNGAEF | ERPLQRVEID | | | | (SEQ ID |
| | | | WSESLE | HTPLDLILLD | | | | NO: 84) |
| | | | HFCLEA | DETLHPLGR | | | | |
| | | | GINIQY | PYLTILKDSLS | | | | |
| | | | NKVGQ | KCIIGYHLSF | | | | |
| | | | PWGKG | QAPSYASAS | | | | |
| | | | LVERNF | KAICHAMLP | | | | |
| | | | LTIQQLI | KKIKGPDGK | | | | |
| | | | LDDLEG | PSWECHGKI | | | | |
| | | | KTFSNN | ETLVADNGA | | | | |
| | | | VERADY | EFWSESLEH | | | | |
| | | | NSVKNA | FCLEAGINIQ | | | | |
| | | | KFKFSRF | YNKVGQPW | | | | |
| | | | VKAFET | GKGLVERNF | | | | |
| | | | WVAEV | LTIQQLILDD | | | | |
| | | | FNWEP | LEGKTFSNN | | | | |
| | | | NQKKT | VERADYNSV | | | | |
| | | | HVPML | KNAKFKFSRF | | | | |
| | | | EWRKA | VKAFETWVA | | | | |
| | | | VNKFPP | EVENWEPN | | | | |
| | | | NELTPP | QKKTHVPML | | | | |
| | | | EHEHIKL | EWRKAVNKF | | | | |
| | | | ISGILKK | PPNELTPPEH | | | | |
| | | | PALQN | EHIKLISGILK | | | | |
| | | | NGIIFEH | KPALQNNGII | | | | |
| | | | LRYDSK | FEHLRYDSKE | | | | |
| | | | ELADYR | LADYRKQFC | | | | |
| | | | KQFCRD | RDKKIKVTTK | | | | |
| | | | KKIKVTT | VNIDDLGFA | | | | |
| | | | KVNIDD | YVYLFEYERY | | | | |
| | | | LGFAYV | LKVPCVDFQ | | | | |
| | | | YLFEYER | YASGLSYEKH | | | | |
| | | | YLKVPC | KVHITYIRKY | | | | |
| | | | VDFQYA | NKIHGKSGL | | | | |
| | | | SGLSYE | DQARAKQHI | | | | |
| | | | KHKVHI | AEILEDIDAS | | | | |
| | | | TYIRKY | AKESSSKQKK | | | | |
| | | | NKIHGK | VGGMKKAA | | | | |
| | | | SGLDQA | RVKGVDSVS | | | | |
| | | | RAKQHI | VQTRREKDS | | | | |
| | | | AEILEDI | NPVKQPSSL | | | | |
| | | | DASAKE | ADLEMIWQ | | | | |
| | | | SSSKQK | EDT* (SEQ | | | | |
| | | | KVGGM | ID NO: 80) | | | | |
| | | | KKAARV | | | | | |
| | | | KGVDSV | | | | | |
| | | | SVQTRR | | | | | |
| | | | EKDSNP | | | | | |
| | | | VKQPSS | | | | | |
| | | | LADLEM | | | | | |
| | | | IWQEDT | | | | | |
| | | | * (SEQ | | | | | |
| | | | ID | | | | | |
| | | | NO: 79) | | | | | |
| 14 | V. | MYHTFE | MSDNS | MYHTFESLL | MTASVKML | MTASVKML | MFLQRPKPY | MPKKKRKV |
| | angui | SLLQV | EDVHAF | QVWLFDMK | HQQVKNIFIS | HQQVKNIFI | SDESLESFFIR | GSGEQKLIS |
| | llarum | WLFDM | GGFFSE | KRILKNSKVK | DAQIDEILAD | SDAQIDEIL | VANKNGYD | EEDLEQKLI |
| | J360_ | KKRILKN | KSSVISV | NISRFVSLKT | IDECREDSDR | ADIDECRED | DVHRFLEAT | SEEDLEQKL |
| | AZS27374. | SKVKNI | PKTSKG | DSVQTTESD | ISEPECLIVVG | SDRISEPEC | KRFLQDIDH | ISEEDLGSG |
| | 1 | SRFVSL | APFGTE | LEFDACFHFE | DSGSGKTTII | LIVVGDSGS | HGYQTFPTD | FLQRPKPYS |
| | | KTDSVQ | LQERYQ | FAPQIKTFET | DKYLSDNPR | GKTTIIDKYL | ITRINPCSAN | DESLESFFIR |
| | | TTESDL | DLFSFD | QPLGFKYRM | MEANDGSII | SDNPRMEA | NSSRARTASL | VANKNGYD |
| | | EFDACF | EKRRDE | NGRLRRYTP | PILFTSLPAN | NDGSIIPILF | LKLAQLTFNE | DVHRFLEA |
| | | HFEFAP | AIHRYNI | DMLCYFHD | ANPVTASER | TSLPANAN | QPELLGLALN | TKRFLQDID |
| | | QIKTFET | LDYLIEL | GYAPYYEVK | LLSSMGDPL | PVTASERLL | RTNLQYSPST | HHGYQTFP |
| | | QPLGFK | HGPSLT | PKWVTEQD | AFNHGKDPA | SSMGDPLA | SAVIRGAEVL | TDITRINPC |
| | | YRMNG | LKKILGS | EFKEKFDAQ | ELMKIVKDLL | FNHGKDPA | PRSLLRTNSIS | SANNSSRA |
| | | RLRRYT | MKGLE | RQQAIANGH | RECRVELIIID | ELMKIVKDL | SCPLCLQEN | RTASLLKLA |
| | | PDMLC | DKFYPN | DLLVLTEEDI | EFQHMIDRK | LRECRVELIII | GYASYLWHF | QLTFNEQP |
| | | YFHDGY | VPSPPSI | QIYPLLDNLK | SKDVLHITAD | DEFQHMID | KGYDHCHIH | ELLGLALNR |
| | | APYYEV | YRYWN | IIHRYACSDN | WLKMIIIESKI | RKSKDVLHI | NIPLINACSC | TNLQYSPST |
| | | KPKWV | TYKKSG | LDDVQIRLLK | PVVLFGMPY | TADWLKMI | GAEFDYRVC | SAVIRGAEV |
| | | TEQDEF | FVLSYLV | LFQNYGEMR | STEILRANNQ | IIESKIPVVLF | GLKGICNNC | LPRSLLRTN |
| | | KEKFDA | PGVTSG | ISQVLKASQ | LRGRFESQH | GMPYSTEIL | KEPITTKNQE | SISSCPLCLQ |
| | | QRQQAI | NRAPRK | GQSASILPAL | HLKPFKVKKT | RANNQLRG | NSYEATSTVS | ENGYASYL |
| | | ANGHD | ALELEEY | YDLIAKKILEF | SERIRYKTFLT | RFESQHHL | NWLAGNGS | WHFKGYD |
| | | LLVLTEE | IDNAIKS | DWHCPISHD | MLDAALPFS | KPFKVKKTS | QDLPDIPRSY | HCHIHNIPLI |
| | | DIQIYPL | YFSEESP | SLIWRVSGS | TKSGLASEDL | ERIRYKTFLT | RWGLIHWW | NACSCGAE |
| | | LDNLKII | TIQQAF | GPKKKRKVG | MKRVYVFSK | MLDAALPF | MNLNDNEF | FDYRVCGL |
| | | HRYACS | TLLEVEL | SGYPYDVPD | GNMRLIRRLI | STKSGLASE | DHLSFTHFFS | KGICNNCKE |
| | | DNLDD | DRHNE | YAYPYDVPD | NKAAKFALLE | DLMKRVYV | NWPRSFHS | PITTKNQEN |
| | | VQIRLLK | CNDTQL | YAYPYDVPD | NAPCISLMH | FSKGNMRL | MIDDEIEFNL | SYEATSTVS |
| | | LFQNYG | TFEYESF | YAGSGSDNS | FARAAPKVS | IRRLINKAA | EHAVVSTSEL | NWLAGNG |
| | | EMRISQ | RKRIVK | EDVHAFGGF | RDACESFNP | KFALLENAP | RLKDLLGRLF | SQDLPDIPR |
| | | VLKASQ | KPDYER | FSEKSSVISVP | FDVDIKQLKII | CISLMHFAR | FHSIRLPERN | SYRWGLIH |
| | | GQSASI | LLIKKGK | KTSKGAPFG | EPSDDVGW | AAPKVSRD | LQHNIILGEL | WWMNLN |
| | | LPALYD | KAADTF | TELQERYQD | ENYLAAKGD | ACESFNPFD | LSHLEKRLW | DNEFDHLS |
| | | LIAKKIL | YKKVGH | LFSFDEKRRD | * (SEQ ID | VDIKQLKIIE | RDKGLIANLK | FTHFFSNW |
| | | EFDWH | RPETTR | EAIHRYNILD | NO: 88) | PSDDVGSG | MNALEASV | PRSFHSMI |
| | | CPISHD | VLQRVE | YLIELHGPSLT | | PAAKKKKL | MLNCSLEQI | DDEIEFNLE |
| | | SLIWRV | ADHTRL | LKKILGSMK | | DGSGGWE | ASMVEQRIL | HAVVSTSEL |
| | | S (SEQ | DLFVID | GLEDKFYPN | | NYLAAKGD | KPNRRTKPN | RLKDLLGRL |
| | | ID | DARTLP | VPSPPSIYRY | | GPH* (SEQ | SPIETTDYLF | FFHSIRLPER |
| | | NO: 85) | LGRPW | WNTYKKSGF | | ID NO: 89) | HFGDIFCLW | NLQHNIILG |
| | | | LTLLFDT | VLSYLVPGVT | | | LAEFQTDEF | ELLSHLEKR |
| | | | HTKSVV | SGNRAPRKA | | | NRSFYVSRW | LWRDKGLI |
| | | | GFYLGF | LELEEYIDNAI | | | * (SEQ ID | ANLKMNAL |
| | | | EPPSYLS | KSYFSEESPTI | | | NO: 90) | EASVMLNC |
| | | | VSLALE | QQAFTLLEV | | | | SLEQIASMV |
| | | | NAILPK | ELDRHNECN | | | | EQRILKPNR |
| | | | DYVKEL | DTQLTFEYES | | | | RTKPNSPIE |
| | | | YPDVKN | FRKRIVKKPD | | | | TTDYLFHFG |
| | | | EWPCY | YERLLIKKGK | | | | DIFCLWLAE |
| | | | GLPEHLI | KAADTFYKK | | | | FQTDEFNR |
| | | | VDNGA | VGHRPETTR | | | | SFYVSRW* |
| | | | EFNSKD | VLQRVEADH | | | | (SEQ ID |
| | | | FVSACK | TRLDLFVIDD | | | | NO: 91 |
| | | | NLRIKV | ARTLPLGRP | | | | |
| | | | KKNPVK | WLTLLFDTH | | | | |
| | | | KPWLK | TKSVVGFYL | | | | |
| | | | GSVERY | GFEPPSYLSV | | | | |
| | | | FRTINN | SLALENAILP | | | | |
| | | | KLLSGIP | KDYVKELYP | | | | |
| | | | GKSFSN | DVKNEWPC | | | | |
| | | | IFARGD | YGLPEHLIVD | | | | |
| | | | YNPQK | NGAEFNSKD | | | | |
| | | | NAIITRS | FVSACKNLRI | | | | |
| | | | DLMKVI | KVKKNPVKK | | | | |
| | | | HVWLID | PWLKGSVER | | | | |
| | | | IYQSSP | YFRTINNKLL | | | | |
| | | | NGLETN | SGIPGKSFSN | | | | |
| | | | IPNLTW | IFARGDYNP | | | | |
| | | | ADAMR | QKNAIITRSD | | | | |
| | | | SALPPR | LMKVIHVWL | | | | |
| | | | PFKGTI | IDIYQSSPNG | | | | |
| | | | DELRFN | LETNIPNLT | | | | |
| | | | LGKNAE | WADAMRSA | | | | |
| | | | ISLDKN | LPPRPFKGTI | | | | |
| | | | GIRFKKT | DELRFNLGK | | | | |
| | | | LRYSSAS | NAEISLDKN | | | | |
| | | | LAQYFG | GIRFKKTLRY | | | | |
| | | | KHTYDG | SSASLAQYFG | | | | |
| | | | KSIKVKI | KHTYDGKSIK | | | | |
| | | | KYDPTC | VKIKYDPTC | | | | |
| | | | MGKIYV | MGKIYVLDE | | | | |
| | | | LDEDKH | DKHEFFAVE | | | | |
| | | | EFFAVE | SVDPDYAYS | | | | |
| | | | SVDPDY | VSEWLHKVC | | | | |
| | | | AYSVSE | CDYARDHIR | | | | |
| | | | WLHKV | NNYRHHDVI | | | | |
| | | | CCDYAR | KAWRVIYDII | | | | |
| | | | DHIRNN | YEALHLSGN | | | | |
| | | | YRHHD | DKQTNIGIRE | | | | |
| | | | VIKAWR | ASKFERVRE | | | | |
| | | | VIYDIIYE | HSERTKSQK | | | | |
| | | | ALHLSG | RPELSYIDED | | | | |
| | | | NDKQT | DIDWGIDVD | | | | |
| | | | NIGIREA | TDGWKIDSV | | | | |
| | | | SKFERV | RGNQL* | | | | |
| | | | REHSER | (SEQ ID | | | | |
| | | | TKSQKR | NO: 87) | | | | |
| | | | PELSYID | | | | | |
| | | | EDDID | | | | | |
| | | | WGIDV | | | | | |
| | | | DTDGW | | | | | |
| | | | KIDSVR | | | | | |
| | | | GNQL* | | | | | |
| | | | (SEQ ID | | | | | |
| | | | NO: 86) | | | | | |
| 15 | Halo- | MYRRKL | MLEDPF | MYRRKLRHS | MNIIHSECN | MNIIHSECN | MKLLVRPRP | MPKKKRKV |
| | monas | RHSRVK | FDESLA | RVKNLYKFAS | QRRLYKFLN | QRRLYKFLN | FINESLESYM | GSGEQKLIS |
| | sp. Salt | NLYKFA | GIGFSH | FKTATAHTV | CFVQHAAM | CFVQHAA | LRLSQENFFE | EEDLEQKLI |
| | Lake7 | SFKTAT | TACKKS | ESSLEFDACY | KKTLNSLYRL | MKKTLNSL | YYQQLSRAIK | SEEDLEQKL |
| | | AHTVES | RDEIED | HFEYSPHVKS | KNNQILGGE | YRLKNNQIL | DWLQLHDH | ISEEDLGSG |
| | | SLEFDA | VAFITID | FIAQPMGFT | QQCMLITGD | GGEQQCM | EAAGAFPEE | KLLVRPRPFI |
| | | CYHFEY | DLDEEC | YSIHGKTNPY | TGSGKSALIK | LITGDTGSG | LSRLNVYHA | NESLESYML |
| | | SPHVKS | ADKALF | TPDFKIINNN | EFSSAFPSYE | KSALIKEFSS | AQSSSRRIRA | RLSQENFFE |
| | | FIAQPM | KYKVIKL | QKIAFIEIKPH | ENGVLIQPVL | AFPSYEENG | LKLVESLTDN | YYQQLSRAI |
| | | GFTYSIH | VNKRLN | SKTLHPEFVQ | VSRIPSKPDV | VLIQPVLVS | EKLPLLHLAV | KDWLQLH |
| | | GKTNPY | GGWTK | KFQAKKEAA | EKMMIELM | RIPSKPDVE | MHSSEKFCS | DHEAAGAF |
| | | TPDFKII | KNVEPII | CQLGFELSLV | NDLGQFGSE | KMMIELM | RYSSVFYAGS | PEELSRLNV |
| | | NNNQKI | YELYNE | TELQIRKYPIL | ARKGRRREI | NDLGQFGS | HVPRALVRQ | YHAAQSSS |
| | | AFIEIKP | GFIDKK | NNYKLLHRY | GLAEALVKM | EARKGRRR | KGIPVCPDCL | RRIRALKLV |
| | | HSKTLH | PGWQS | AGFQSHCEL | LKVCKTQIIII | EIGLAEALV | TEANYIRQE | ESLTDNEKL |
| | | PEFVQK | VARWN | YDSVYSLVKR | NEFQELIEFK | KMLKVCKT | WHWMPYE | PLLHLAVM |
| | | FQAKKE | AKYRVD | HSPIFLHEICA | SVEDRQRIA | QIIIINEFQE | ACINHGKQ | HSSEKFCSR |
| | | AACQLG | KNLLYL | LYDIGFRPRV | NRLKLISEQA | LIEFKSVED | MLHECPKCE | YSSVFYAGS |
| | | FELSLVT | VDRRAI | IRSLVSLIASG | GIPVVLVGM | RQRIANRLK | EKLNYTHSEC | HVPRALVR |
| | | ELQIRKY | KNNFCD | KLKANILEKEI | PWASEISNE | LISEQAGIP | LHTCRCGFD | QKGIPVCP |
| | | PILNNY | FNSFSK | GDDLLLWA | PQWASRLM | VVLVGMP | LRNADTEPA | DCLTEANYI |
| | | KLLHRY | DTFFW | GSGPKKKRK | CKIELPYFKFL | WASEISNE | DEWQLIASR | RQEWHW |
| | | AGFQS | DAIEKK | VGSGYPYDV | NEDDRKEFT | PQWASRL | LVVGEPSPS | MPYEACIN |
| | | HCELYD | YLTRVR | PDYAYPYDV | CFVKGLACR | MCKIELPYF | NHPLLDIRSV | HGKQMLH |
| | | SVYSLV | GSVATT | PDYAYPYDV | MGYEKPPKF | KFLNEDDR | SLRLACLLWY | ECPKCEEKL |
| | | KRHSPIF | YQFYKD | PDYAGSGLE | EIDEILFPLFS | KEFTCFVKG | QLYAYKTLD | NYTHSECLH |
| | | LHEICAL | LILIHNN | DPFFDESLA | ATRGEARKV | LACRMGYE | ASDQVPTLTI | TCRCGFDLR |
| | | YDIGFR | ENPDN | GIGFSHTACK | KHILSEALSL | KPPKFEIDEI | ERAIEYFTH | NADTEPAD |
| | | PRVIRSL | KFVAVG | KSRDEIEDVA | ALWRGENT | LFPLFSATR | WPEVFTQEL | EWQLIASRL |
| | | VSLIASG | RSAFYD | FITIDDLDEE | VHQQHLAEV | GEARKVKHI | EQQAALSGD | VVGEPSPS |
| | | KLKANIL | RVKKLP | CADKALFKY | MDSAFFYED | LSEALSLAL | KLVCDYNKT | NHPLLDIRS |
| | | EKEIGD | PYICDLK | KVIKLVNKRL | NPFKLPLNEV | WRGENTV | SLRDVFGNIV | VSLRLACLL |
| | | DLLLWA | RYGKRY | NGGWTKKN | PLCEVSKYAS | HQQHLAEV | GISRLLLKAY | WYQLYAYK |
| | | * (SEQ | ADKKYR | VEPIIYELYNE | YNRYSTVES | MDSAFFYE | PESDFVLTPL | TLDASDQV |
| | | ID | LINSFKK | GFIDKKPGW | DMFVSTQFT | DNPFKLPLN | ENFLVRLVD | PTLTIERAIE |
| | | NO: 92) | STRVME | QSVARWNA | PKIPTKVLFS | EVPLCEVSK | QNPQSRVP | YFTHWPEV |
| | | | RVEIDH | KYRVDKNLL | KS* (SEQ ID | YAGSGPAA | NVADLLISM | FTQELEQQ |
| | | | TALDLIL | YLVDRRAIKN | NO: 95) | KKKKLDGS | PEAAILLGTS | AALSGDKL |
| | | | LDDTLN | NFCDFNSFS | | GSYNRYSTV | YEQAYRLYEE | VCDYNKTSL |
| | | | IPIGRPF | KDTFFWDAI | | ESDMFVST | GYLKCAVKF | RDVFGNIV |
| | | | ITVLIDT | EKKYLTRVRG | | QFTPKIPTK | KSHEKLVNGI | GISRLLLKAY |
| | | | FSKCIV | SVATTYQFYK | | VLFSKS* | GVFYLREIME | PESDFVLTP |
| | | | GFYLSF | DLILIHNNEN | | (SEQ ID | LRQSRMPVE | LENFLVRLV |
| | | | RGPSYN | PDNKFVAVG | | NO: 96) | TSSYNNYLPA | DQNPQSRV |
| | | | SVRCAII | RSAFYDRVK | | | W* (SEQ ID | PNVADLLIS |
| | | | NACLDK | KLPPYICDLK | | | NO: 97) | MPEAAILLG |
| | | | EDVLKK | RYGKRYADK | | | | TSYEQAYRL |
| | | | YPDVEK | KYRLINSFKK | | | | YEEGYLKCA |
| | | | DWPCQ | STRVMERVE | | | | VKFKSHEKL |
| | | | GRIETLV | IDHTALDLILL | | | | VNGIGVFYL |
| | | | VDNGA | DDTLNIPIGR | | | | REIMELRQS |
| | | | EFWSK | PFITVLIDTFS | | | | RMPVETSS |
| | | | DLERFS | KCIVGFYLSF | | | | YNNYLPAW |
| | | | ASIGMS | RGPSYNSVR | | | | * (SEQ ID |
| | | | IEYNPV | CAIINACLDK | | | | NO: 98) |
| | | | GKPWK | EDVLKKYPD | | | | |
| | | | KPLVERI | VEKDWPCQ | | | | |
| | | | FNTYNT | GRIETLVVDN | | | | |
| | | | KFVHQI | GAEFWSKDL | | | | |
| | | | PGKTFS | ERFSASIGMS | | | | |
| | | | SAKDLE | IEYNPVGKP | | | | |
| | | | GYEPQK | WKKPLVERIF | | | | |
| | | | DALLPF | NTYNTKFVH | | | | |
| | | | SEFLYLL | QIPGKTFSSA | | | | |
| | | | HIWVID | KDLEGYEPQ | | | | |
| | | | IYNQQS | KDALLPFSEF | | | | |
| | | | NSRKTH | LYLLHIWVIDI | | | | |
| | | | IPALSW | YNQQSNSRK | | | | |
| | | | QVGYEE | THIPALSWQ | | | | |
| | | | FPPVIY | VGYEEFPPVI | | | | |
| | | | QGLEKQ | YQGLEKQRF | | | | |
| | | | RFKIESF | KIESFPTVYR | | | | |
| | | | PTVYRD | DLRPIGIEVD | | | | |
| | | | LRPIGIE | HISYSNEALV | | | | |
| | | | VDHISY | EFRKNNPPP | | | | |
| | | | SNEALV | LGQTKHKLC | | | | |
| | | | EFRKNN | VKRDPSDVS | | | | |
| | | | PPPLGQ | YVYVYLPNLE | | | | |
| | | | TKHKLC | KYIKVDATSQ | | | | |
| | | | VKRDPS | DFSLEGVSIF | | | | |
| | | | DVSYVY | QYQVMRKA | | | | |
| | | | VYLPNL | LTRYIDANVD | | | | |
| | | | EKYIKV | HAGLALAN | | | | |
| | | | DATSQ | MKLSERMD | | | | |
| | | | DFSLEG | DISNLALANK | | | | |
| | | | VSIFQY | KSRSRGMKS | | | | |
| | | | QVMRK | VAAFVGIDSE | | | | |
| | | | ALTRYID | GETSFESVH | | | | |
| | | | ANVDH | NNLKNKKDT | | | | |
| | | | AGLALA | KLSFFEGETL | | | | |
| | | | NMKLSE | DDKKLKSIDD | | | | |
| | | | RMDDIS | WNEIADNLE | | | | |
| | | | NLALAN | PY* (SEQ ID | | | | |
| | | | KKSRSR | NO: 94) | | | | |
| | | | GMKSV | | | | | |
| | | | AAFVGI | | | | | |
| | | | DSEGET | | | | | |
| | | | SFESVH | | | | | |
| | | | NNLKNK | | | | | |
| | | | KDTKLS | | | | | |
| | | | FFEGET | | | | | |
| | | | LDDKKL | | | | | |
| | | | KSIDDW | | | | | |
| | | | NEIADN | | | | | |
| | | | LEPY* | | | | | |
| | | | (SEQ ID | | | | | |
| | | | NO: 93) | | | | | |
| 16 | V.EJY3- | MYPHTI | MSGPF | MYPHTIDKP | MTSNSENV | MTSNSENV | VITNIQLYPD | MPKKKRKV |
| | NC_ | DKPHAK | VDESKG | HAKKNIFKFI | QRLVSNFNQ | QRLVSNFN | ESLESFLLRLS | GSGEQKLIS |
| | 016614 | KNIFKFI | EPPNN | SVKNKAIIMC | SFALFPPFDA | QSFALFPPF | QEQGYERFS | EEDLEQKLI |
| | | SVKNKA | GEGGLV | ESSLEFDACF | ILSDLEKLRN | DAILSDLEK | HFAEDIWYQ | SEEDLEQKL |
| | | IIMCESS | QDVNIG | HLEYHPDVA | KSAREGFKPS | LRNKSARE | TLNENEAMS | ISEEDLGSGI |
| | | LEFDAC | DVTTDS | SFESQPFYLE | MLIYGDTGA | GFKPSMLIY | GAFPLELNCI | TNIQLYPDE |
| | | FHLEYH | SDLCYR | YQLEDGSHS | GKSALLEHFT | GDTGAGKS | NIYHGHTTSE | SLESFLLRLS |
| | | PDVASF | PLNTLT | YTPDFLVTLN | KESKSKTGRK | ALLEHFTKE | MRARVLIDL | QEQGYERF |
| | | ESQPFY | VYERDL | DGKKYLQEV | VLRTRVRPSL | SKSKTGRKV | ERRIKLNDFG | SHFAEDIW |
| | | LEYQLE | DSFPEE | KNSKLCLTPE | QETLSWTLH | LRTRVRPSL | VLRLALMHS | YQTLNENE |
| | | DGSHSY | LKNEAL | YLHVFEAMQ | VLNPLRRNN | QETLSWTL | KANFSPKFK | AMSGAFPL |
| | | TPDFLV | ERFKLLS | RGSEDIGFPL | RFVKNASEIG | HVLNPLRR | AVHRFGMD | ELNCINIYH |
| | | TLNDGK | LIGKEFD | YLVTERQIRK | LTDMLIRELK | NNRFVKNA | YPFSFLRKRF | GHTTSEMR |
| | | KYLQEV | GPWPF | AFILDNLKLIH | QANIGIMIID | SEIGLTDML | TPICPMCLG | ARVLIDLER |
| | | KNSKLC | KQIQKLI | RYAGSKHICS | ECQEFVEIRS | IRELKQANI | DAPYIRQNW | RIKLNDFGV |
| | | LTPEYL | EKYKND | FKQSLLEVIQ | NDDKKEISIR | GIMIIDECQ | QFIPVQSCAE | LRLALMHS |
| | | HVFEA | VSIPTPS | NQGLSSSEKL | LKMISEEASV | EFVEIRSND | HGCKLLHQC | KANFSPKFK |
| | | MQRGS | PRTVQR | AGIFGKSIGF | SMIFVGMP | DKKEISIRLK | PECGCRLEY | AVHRFGM |
| | | EDIGFPL | WRERY | MNRELLELM | WSKEITRDS | MISEEASVS | QNSERIQYC | DYPFSFLRK |
| | | YLVTER | EKSNGD | SLGLVSAHFE | QWESRIRLV | MIFVGMP | ECGSNLAEA | RFTPICPMC |
| | | QIRKAFI | LKSLIVR | TSMFDERTA | REIPYFKVINE | WSKEITRDS | EAKVSFESEL | LGDAPYIRQ |
| | | LDNLKLI | NYAKG | IWVSEQVGS | NGSNNKKE | QWESRIRL | MVARWLAG | NWQFIPVQ |
| | | HRYAGS | NRKPKII | GPKKKRKVG | MKRFALSLM | VREIPYFKVI | KSPMEEGV | SCAEHGCK |
| | | KHICSFK | GDEYYF | SGYPYDVPD | EISKLMPLDK | NENGSNNK | MSKDMTTS | LLHQCPEC |
| | | QSLLEVI | DLAVQS | YAYPYDVPD | QPQLELPEFS | KEMKRFAL | ERYGFLLWY | GCRLEYQN |
| | | QNQGL | WLEAER | YAYPYDVPD | FPLLAYSRGE | SLMEISKLM | VNRYGDLED | SERIQYCEC |
| | | SSSEKLA | PNITRA | YAGSGSGPF | MRALKDILS | PLDKQPQL | ISFHAFVKYC | GSNLAEAE |
| | | GIFGKSI | YERYCD | VDESKGEPP | DALEIALNEG | ELPEFSFPLL | AEWPKPLHH | AKVSFESEL |
| | | GFMNR | SIEVAN | NNGEGGLV | AKELTRYHL | AYSRGEMR | ELDKLVDKA | MVARWLA |
| | | ELLELM | ESIVVG | QDVNIGDVT | QEAAKFSVE | ALKDILSDA | DVIRVKQWR | GKSPMEEG |
| | | SLGLVS | KIPSASY | TDSSDLCYRP | GENPFDEKV | LEIALNEGA | KVFFREVFGE | VMSKDMT |
| | | AHFETS | KSFSRRL | _NTLTVYERD | NMIQIQTIQ | KELTRYHLQ | LLKECRELPS | TSERYGFLL |
| | | MFDER | KQLPPY | LDSFPEELKN | QYTRFELDD | EAAKFSVE | RQLSKNIVLV | WYVNRYG |
| | | TAIWVS | AVALQR | EALERFKLLSL | KTGRRERFD | GENPFDEK | EILHYLTRLV | DLEDISFHA |
| | | EQV* | HGKYFA | GKEFDGPW | RAFTALQQIP | VNMIQIQTI | ADSSSSPKG | FVKYCAEW |
| | | (SEQ ID | DLWFR | PFKQIQKLIE | INKLLSKR* | QQYTGSGP | NIADVLLSPF | PKPLHHELD |
| | | NO: 99) | HNAKH | KYKNDVSIPT | (SEQ ID | AAKKKKLD | EASTLLSCST | KLVDKADVI |
| | | | KPPTRIL | PSPRTVQRW | NO: 102) | GSGRFELD | DEVYRLYNF | RVKQWRK |
| | | | ERVEID | RERYEKSNG | | DKTGRRER | GEIQAAFRP | VFFREVFGE |
| | | | HTQLDL | DLKSLIVRNY | | FDRAFTAL | KIHTKLARHE | LLKECRELP |
| | | | MLLHD | AKGNRKPKII | | QQIPINKLL | PVFTLRGMI | SRQLSKNIV |
| | | | EYLVPIG | GDEYYFDLA | | SKR* (SEQ | ETKLVRMCS | LVEILHYLTR |
| | | | RPCLTM | VQSWLEAER | | ID NO: 103) | ESDGLSVYLS | LVADSSSSP |
| | | | LIDVFSG | PNITRAYERY | | | NW (SEQ ID | KGNIADVLL |
| | | | CIIGFHL | CDSIEVANES | | | NO: 104) | SPFEASTLLS |
| | | | GFHAP | IVVGKIPSAS | | | | CSTDEVYRL |
| | | | GYATVA | YKSFSRRLKQ | | | | YNFGEIQA |
| | | | KALLNA | LPPYAVALQ | | | | AFRPKIHTK |
| | | | MKPKD | RHGKYFADL | | | | LARHEPVFT |
| | | | YVKDLPI | WFRHNAKH | | | | LRGMIETKL |
| | | | ELNNE | KPPTRILERV | | | | VRMCSESD |
| | | | WICEGK | EIDHTQLDL | | | | GLSVYLSN |
| | | | IEKLVM | MLLHDEYLV | | | | W (SEQ ID |
| | | | DNGAEF | PIGRPCLTML | | | | NO: 105) |
| | | | WSKSID | IDVFSGCIIGF | | | | |
| | | | DACKEL | HLGFHAPGY | | | | |
| | | | NIAVQY | ATVAKALLN | | | | |
| | | | NPVKKP | AMKPKDYVK | | | | |
| | | | WLKPFI | DLPIELNNE | | | | |
| | | | ERSFGIL | WICEGKIEKL | | | | |
| | | | NKTLLS | VMDNGAEF | | | | |
| | | | TIPGKTF | WSKSIDDAC | | | | |
| | | | SNVLEK | KELNIAVQY | | | | |
| | | | GDYDA | NPVKKPWLK | | | | |
| | | | ANKAV | PFIERSFGILN | | | | |
| | | | MKFSTF | KTLLSTIPGKT | | | | |
| | | | VEELHR | FSNVLEKGD | | | | |
| | | | WIIDVH | YDAANKAV | | | | |
| | | | NAKPDS | MKFSTFVEEL | | | | |
| | | | RNNRLP | HRWIIDVHN | | | | |
| | | | NLYWS | AKPDSRNNR | | | | |
| | | | QGVKTL | LPNLYWSQG | | | | |
| | | | PPARLPI | VKTLPPARLP | | | | |
| | | | KDSEQL | IKDSEQLSII | | | | |
| | | | SIIMGIL | MGILVKRKLT | | | | |
| | | | VKRKLT | EKGIQYEDLF | | | | |
| | | | EKGIQY | YRSQALADY | | | | |
| | | | EDLFYR | RARFPQTKE | | | | |
| | | | SQALAD | SAIKTIKVDP | | | | |
| | | | YRARFP | DDLSRIFIFLE | | | | |
| | | | QTKESA | ELNGYIKVPC | | | | |
| | | | IKTIKVD | DDPEGYTKH | | | | |
| | | | PDDLSR | LSLHEHIIIKR | | | | |
| | | | IFIFLEEL | AHKQYIKGH | | | | |
| | | | NGYIKV | VDTLSLAKAR | | | | |
| | | | PCDDPE | LALAARMEE | | | | |
| | | | GYTKHL | ETEELRSFKR | | | | |
| | | | SLHEHII | KRKPPKNIKK | | | | |
| | | | IKRAHK | MAEYSGLSS | | | | |
| | | | QYIKGH | AAIESKSPAL | | | | |
| | | | VDTLSL | DKMSSRKSN | | | | |
| | | | AKARLA | AEEPKDIANF | | | | |
| | | | LAARM | LDDWEAILG | | | | |
| | | | EEETEEL | DLSDD* | | | | |
| | | | RSFKRK | (SEQ ID | | | | |
| | | | RKPPKN | NO: 101 | | | | |
| | | | IKKMAE | | | | | |
| | | | YSGLSS | | | | | |
| | | | AAIESKS | | | | | |
| | | | PALDK | | | | | |
| | | | MSSRKS | | | | | |
| | | | NAEEPK | | | | | |
| | | | DIANFL | | | | | |
| | | | DDWEA | | | | | |
| | | | ILGDLSD | | | | | |
| | | | D* (SEQ | | | | | |
| | | | ID | | | | | |
| | | | NO: 100) | | | | | |
| 17 | Photo_ | MYIRNL | MAGRF | MYIRNLRKP | MPDSNLELSI | MPDSNLEL | MNTDIQFYP | MPKKKRKV |
| | aquae | RKPSPN | KDEFDA | SPNKNIYKFA | DTTLATYHA | SIDTTLATY | DESLESFLLRL | GSGEQKLIS |
| | CGMCC | KNIYKF | NYSEDD | SSKNRKTVM | SFTIYPEVEK | HASFTIYPE | SHHQGYERF | EEDLEQKLI |
| | | ASSKNR | EKEFLES | CEGGLEKDC | VFSGLDWLV | VEKVFSGLD | AYFAEDIWY | SEEDLEQKL |
| | | KTVMC | PESKRN | CYHFEYDPE | KRRCFGSFV | WLVKRRCF | QTRDQHEAI | ISEEDLGSG |
| | | EGGLEK | RLQYGS | VVCYESQPE | PSMLLTGGT | GSFVPSML | AGAFPLELN | NTDIQFYPD |
| | | DCCYHF | LDSAKII | GYYYEFCGK | GSGKSALIKH | LTGGTGSG | RVNVYHAHT | ESLESFLLRL |
| | | EYDPEV | ERDLDS | QLPYTPDFLV | YISKCLSENE | KSALIKHYIS | TSQMRVRVL | SHHQGYER |
| | | VCYESQ | FPEEQK | HYIGGYQCF | VLLTRVRPTL | KCLSENEVL | MHLENQLNL | FAYFAEDI |
| | | PEGYYY | TKALER | VESKPYGQT | KETLLWIVNE | LTRVRPTLK | DDFRVLHIVL | WYQTRDQ |
| | | EFCGKQ | YKLLSLV | LSKEFKQQF | IDKYKKYRAK | ETLLWIVNE | AHSKSQFSP | HEAIAGAFP |
| | | LPYTPD | SKELVG | QARKSAAER | GSVLGLIDYV | IDKYKKYRA | DFKAVHRCG | LELNRVNV |
| | | FLVHYI | GWTPK | LGFDLILVTD | IRCVKRTELK | KGSVLGLID | VDYPFAFLRK | YHAHTTSQ |
| | | GGYQCF | NLNPLI | RQIRKGYYLE | LLVIEECQELF | YVIRCVKRT | RFMPVCPLC | MRVRVLM |
| | | VESKPY | DKYFEK | NCKVVHRYS | ECTSHKERQ | ELKLLVIEEC | LAESAYVRQ | HLENQLNL |
| | | GQTLSK | TTLTQK | GCIKGDNLP | EIRDKLKMIS | QELFECTSH | HWHFIPIQA | DDFRVLHIV |
| | | EFKQQF | PSYKTLI | DALYDQLLD | DECRLPIVFV | KERQEIRDK | CEQHGCKLI | LAHSKSQFS |
| | | QARKSA | RWHNS | TKPIKIIDLAL | GIPSAKLILED | LKMISDECR | HRCPACDGL | PDFKAVHR |
| | | AERLGF | FNQAK | KVELSVGVV | SQWDRRIM | LPIVFVGIPS | LEYQSTECM | CGVDYPFA |
| | | DLILVTD | GSFTGL | FAAVLRLVTL | VKRELPYFKI | AKLILEDSQ | THCECGFNL | FLRKRFMP |
| | | RQIRKG | VDKHH | GKALIDLDSA | TDEASIDRYL | WDRRIMV | LSTPTTSASA | VCPLCLAES |
| | | YYLENC | QKGNR | KLNETTLVM | DLLEAMERA | KRELPYFKIT | SELLISRWLT | AYVRQHW |
| | | KVVHRY | TARVVG | VKGSGPKKK | VPLPFDVDL | DEASIDRYL | GTQLDVAGL | HFIPIQACE |
| | | SGCIKG | DESYYE | RKVGSGYPY | MDVEIAMRL | DLLEAMER | MGKALSISER | QHGCKLIH |
| | | DNLPDA | KALERF | DVPDYAYPY | LAASHGMLG | AVPLPFDV | YGFLLWYVN | RCPACDGL |
| | | LYDQLL | LDAVRP | DVPDYAYPY | MLKELIAVGL | DLMDVEIA | RYGDLEDISF | LEYQSTEC |
| | | DTKPIKII | SIRAAY | DVPDYAGSG | ESALISNKAA | MRLLAASH | DVFVEYCTT | MTHCECGF |
| | | DLALKV | NVYCD | AGRFKDEFD | IQLEDFILGYE | GMLGMLK | WPKSLRKDL | NLLSTPTTS |
| | | ELSVGV | NITVAN | ANYSEDDEK | MIFGLDEINP | ELIAVGLES | DECVQKADA | ASASELLISR |
| | | VFAAVL | ENIVSG | EFLESPESKR | FSVDINELVI | ALISNKAAI | IRVKRWKQV | WLTGTQLD |
| | | RLVTLG | KIPQVS | NRLQYGSLD | KQIESYEEYV | QLEDFILGY | FFSEAFGALL | VAGLMGK |
| | | KALIDL | YQTFKN | SAKIIERDLDS | PDAETGELKF | EMIFGLDEI | KGCRQLPSR | ALSISERYG |
| | | DSAKLN | RIQKEQ | FPEEQKTKAL | VGQIFNALTI | NPFSVDINE | QLSKNCVLV | FLLWYVNR |
| | | ETTLVM | PYSVAL | ERYKLLSLVS | KQLLG* | LVIKQIESYE | EILNYFKRLV | YGDLEDISF |
| | | VK* | ARHGKY | KELVGGWTP | (SEQ ID | GSGPAAKK | ADNPKSSKG | DVFVEYCTT |
| | | (SEQ ID | YADKLY | KNLNPLIDKY | NO: 109) | KKLDGSGE | NIADVLLSPL | WPKSLRKD |
| | | NO: 106) | NYYQSV | FEKTTLTQKP | | YVPDAETG | EASTLLSCTT | LDECVQKA |
| | | | DMPTRI | SYKTLIRWH | | ELKFVGQIF | DEVYRLYEFG | DAIRVKRW |
| | | | LERVEM | NSFNQAKGS | | NALTIKQLL | EIKAAMRPKI | KQVFFSEAF |
| | | | DHTPLD | FTGLVDKHH | | G* (SEQ ID | HTKIASHESA | GALLKGCR |
| | | | LILLHDD | QKGNRTARV | | NO: 110) | FTLRSVVETR | QLPSRQLSK |
| | | | LLVPLG | VGDESYYEK | | | LTRMCSEND | NCVLVEILN |
| | | | RAHLTL | ALERFLDAVR | | | GLSVYLPEW | YFKRLVAD |
| | | | LVDIFSG | PSIRAAYNVY | | | * (SEQ ID | NPKSSKGN |
| | | | CIIGFHL | CDNITVANE | | | NO: 111) | ADVLLSPLE |
| | | | GFKHPS | NIVSGKIPQV | | | | ASTLLSCTT |
| | | | YVSASK | SYQTFKNRIQ | | | | DEVYRLYEF |
| | | | AIIHATK | KEQPYSVAL | | | | GEIKAAMR |
| | | | NKDYIS | ARHGKYYAD | | | | PKIHTKIAS |
| | | | GLPIEFE | KLYNYYQSV | | | | HESAFTLRS |
| | | | NKWLC | DMPTRILER | | | | VVETRLTR |
| | | | EGKIEN | VEMDHTPLD | | | | MCSENDGL |
| | | | LVVDN | LILLHDDLLV | | | | SVYLPEW* |
| | | | GPEFW | PLGRAHLTLL | | | | (SEQ ID |
| | | | SKSLED | VDIFSGCIIGF | | | | NO: 112) |
| | | | SCLEAGI | HLGFKHPSY | | | | |
| | | | NVVFNK | VSASKAIIHA | | | | |
| | | | VRKPW | TKNKDYISGL | | | | |
| | | | LKPFVE | PIEFENKWLC | | | | |
| | | | RKFGEII | EGKIENLVVD | | | | |
| | | | QGIVG | NGPEFWSKS | | | | |
| | | | WVPGK | LEDSCLEAGI | | | | |
| | | | TFSNVL | NVVFNKVRK | | | | |
| | | | EKEDYN | PWLKPFVER | | | | |
| | | | PEKDAV | KFGEIIQGIV | | | | |
| | | | MRFSVF | GWVPGKTFS | | | | |
| | | | VEELHR | NVLEKEDYN | | | | |
| | | | WIVDV | PEKDAVMRF | | | | |
| | | | HNASA | SVFVEELHR | | | | |
| | | | DSRKAR | WIVDVHNAS | | | | |
| | | | IPNLYW | ADSRKARIP | | | | |
| | | | RKSYEV | NLYWRKSYE | | | | |
| | | | MPPLKL | VMPPLKLLP | | | | |
| | | | LPENEH | ENEHTFTIA | | | | |
| | | | TFTIAM | MGSLHHRKL | | | | |
| | | | GSLHHR | TSKGIKFKHI | | | | |
| | | | KLTSKGI | DYDSTALAQ | | | | |
| | | | KFKHID | YRKEYPQTK | | | | |
| | | | YDSTAL | ASAIKKIKVD | | | | |
| | | | AQYRKE | PDDISTIYIYL | | | | |
| | | | YPQTKA | EELNGYVEV | | | | |
| | | | SAIKKIK | PSKDSKGYT | | | | |
| | | | VDPDDI | RKLSLCEHEK | | | | |
| | | | STIYIYLE | LVKAHRDYI | | | | |
| | | | ELNGYV | DGEIDVLSLA | | | | |
| | | | EVPSKD | KARLALHERI | | | | |
| | | | SKGYTR | QSEQENLQH | | | | |
| | | | KLSLCE | MSLSERKRK | | | | |
| | | | HEKLVK | AKATKKIAEL | | | | |
| | | | AHRDYI | SSVNSDTPK | | | | |
| | | | DGEIDV | AQLTDKLPP | | | | |
| | | | LSLAKA | NPSMSGCSG | | | | |
| | | | RLALHE | SPAEKEINPIE | | | | |
| | | | RIQSEQ | NFRSKWNK | | | | |
| | | | ENLQH | RRKERNG* | | | | |
| | | | MSLSER | (SEQ ID | | | | |
| | | | KRKAKA | NO: 108) | | | | |
| | | | TKKIAEL | | | | | |
| | | | SSVNSD | | | | | |
| | | | TPKAQL | | | | | |
| | | | TDKLPP | | | | | |
| | | | NPSMS | | | | | |
| | | | GCSGSP | | | | | |
| | | | AEKEIN | | | | | |
| | | | PIENFRS | | | | | |
| | | | KWNKR | | | | | |
| | | | RKERNG | | | | | |
| | | | * (SEQ | | | | | |
| | | | ID | | | | | |
| | | | NO: 107) | | | | | |
| 18 | Entero- | MYRRN | MNSSD | MYRRNLKHS | MTEIMGDF | MKAMTEE | MSKLSIRIEH | MPKKKRKV |
| | vibrio | LKHSRV | DDDSLP | RVKNLFKFCS | DRLRQNRLL | QSVKLKAFL | RIDESLESYLL | GSGEQKLIS |
| | coralii | KNLFKF | LFSNEFS | LKNGSVLTVE | GGDQQCML | NCFVEYPLL | RLSQANYFES | EEDLEQKLI |
| | strain | CSLKNG | PSSSSEY | SALEFDTCFH | LTGDTGCGK | TEIMGDFD | YQLLSRAVK | SEEDLEQKL |
| | CAIM | SVLTVE | KPNSSP | LEYCKDIVCF | SHLIRYYQSR | RLRQNRLL | DWLYEHDEE | ISEEDLGSG |
| | 912 | SALEFD | PKEPQK | EAQPEGFYY | EQSEPKGRF | GGDQQCM | AFGAFPLQF | SKLSIRIEHR |
| | | TCFHLE | LIERDLD | QFEGKKLPYT | DSSPILVSRIP | LLTGDTGC | KTVNVYHAA | IDESLESYLL |
| | | YCKDIV | SYPAHL | PDFRVSYED | SKLSLEETVL | GKSHLIRYY | QSSGFRVRA | RLSQANYFE |
| | | CFEAQP | KEEAIKR | RREVFLEIKP | QLLKDLGQF | QSREQSEP | LRLIDWLAD | SYQLLSRAV |
| | | EGFYYQ | FRLLAFI | ASKIEGDEFR | GTTTRGRSRI | KGRFDSSPI | TELPLLQLAL | KDWLYEHD |
| | | FEGKKL | NKNLN | RKFVGKMEV | TTDSSLTHSL | LVSRIPSKLS | LGSSTRFCFA | EEAFGAFPL |
| | | PYTPDF | GGWTP | AKSLGCPLSL | VELLRKKQVE | LEETVLQLL | HASVFRQGT | QFKTVNVY |
| | | RVSYED | KNLNPLI | VTDNQIRVN | LIIINEFQELIE | KDLGQFGT | HIPLCFVRKA | HAAQSSGF |
| | | RREVFL | QQHFEE | PVLYNLKLLH | YKSAEKKQAI | TTRGRSRIT | GVPICPECLK | RVRALRLID |
| | | EIKPASK | TGQSDP | RYTGIVGINA | ANRLKYISEE | TDSSLTHSL | ESEHIPQVW | WLADTELP |
| | | IEGDEF | PKSRVV | IQAQLLKVVR | AGVPIVLVG | VELLRKKQV | HFLPYIACHK | LLQLALLGS |
| | | RRKFVG | CNWRK | ASGLVSINDL | MPWAEMIA | ELIIINEFQE | HHLDLIETCP | STRFCFAHA |
| | | KMEVA | SYELSG | SQRVHVSSG | EEPQWSSRL | LIEYKSAEKK | SCGALVDYLT | SVFRQGTHI |
| | | KSLGCP | GKITAL | ELKANALALI | VTRRSLPYFK | QAIANRLKY | SEKVSECECG | PLCFVRKAG |
| | | LSLVTD | VPKHHR | SRGQLQAEL | LSEDPVHFV | ISEEAGVPI | FDLKNAPTH | VPICPECLK |
| | | NQIRVN | KGNYEL | NKEKFGMHS | QFLKGLAKK | VLVGMPW | KADPLRVLLS | ESEHIPQV |
| | | PVLYNL | KNTGD | VVWIGAGSG | MPFDKPPKL | AEMIAEEP | CLAVGDPFD | WHFLPYIAC |
| | | KLLHRY | GAIFHD | PKKKRKVGS | EDKKTSISLF | QWSSRLVT | FDETALGQC | HKHHLDLIE |
| | | TGIVGI | ALERFL | GYPYDVPDY | AASRGELRA | RRSLPYFKL | NQSTRFGAL | TCPSCGALV |
| | | NAIQAQ | NARRPS | AYPYDVPDY | LRHLINDAVK | SEDPVHFV | LWYHLEFVG | DYLTSEKVS |
| | | LLKVVR | MTTAYE | AYPYDVPDY | DAVLEDERE | QFLKGLAKK | NLEQGNAIN | ECECGFDLK |
| | | ASGLVSI | YYKDQI | AGSGNSSDD | FNVQRLHHS | MPFDKPPK | VEGLSGAIGF | NAPTHKAD |
| | | NDLSQR | LLSNERL | DDSLPLFSNE | FTKLNPQVR | LEDKKTSISL | FNKWPESFH | PLRVLLSCL |
| | | VHVSSG | VEGVIK | FSPSSSSEYK | NPFELPLNEI | FAASRGELR | TAMDRRLAT | AVGDPFDF |
| | | ELKANA | PLSYSG | PNSSPPKEP | KLSEIEHYSG | ALRHLINDA | WEASRYIEY | DETALGQC |
| | | LALISRG | FKKRIK | QKLIERDLDS | YNPRAMSN | VKDAVLED | NHTPFRKIFG | NQSTREGA |
| | | QLQAEL | QLPPYQ | YPAHLKEEAI | DDALTNRMF | EREFNVQR | DVLLHSSRLP | LLWYHLEF |
| | | NKEKFG | VAVAR | KRFRLLAFIN | SENIPLKELLK | LHHSFTKLN | SKDLSHNFVL | VGNLEQGN |
| | | MHSVV | HGKFM | KNLNGGWT | KKG* (SEQ | PQVRNPFE | RELLAYLSHL | AINVEGLSG |
| | | WIGA* | ADQWY | PKNLNPLIQ | ID NO: 116) | LPLNEIKLSE | VLRHPKSKT | AIGFFNKW |
| | | (SEQ ID | GYFSAH | QHFEETGQS | | IEHYSGSGP | ANAGDVLLT | PESFHTAM |
| | | NO: 113) | KPPTRIL | DPPKSRVVC | | AAKKKKLD | LSETASLLSTS | DRRLATWE |
| | | | EKVEID | NWRKSYELS | | GSGGYNPR | YEQVERLYQ | ASRYIEYNH |
| | | | HTPLDLI | GGKITALVPK | | AMSNDDAL | EGFLKLIYRP | TPFRKIFGD |
| | | | LIDDELF | HHRKGNYEL | | TNRMFSEN | HQQTTIPPH | VLLHSSRLP |
| | | | VPFGRP | KNTGDGAIF | | IPLKELLKKK | KPAFRLRNVI | SKDLSHNFV |
| | | | YLTLLID | HDALERFLN | | G* (SEQ ID | ELGVARMQ | LRELLAYLS |
| | | | VFSSCIV | ARRPSMTTA | | NO: 117) | TDVSSDVYLP | HLVLRHPKS |
| | | | GFHLGY | YEYYKDQILL | | | AW* (SEQ ID | KTANAGDV |
| | | | KAPSYD | SNERLVEGVI | | | NO: 118) | LLTLSETASL |
| | | | SVSKAII | KPLSYSGFKK | | | | LSTSYEQVE |
| | | | HATKPK | RIKQLPPYQV | | | | RLYQEGFLK |
| | | | DYLDSI | AVARHGKF | | | | LIYRPHQQT |
| | | | ASDFQH | MADQWYGY | | | | TIPPHKPAF |
| | | | DWPCC | FSAHKPPTRI | | | | RLRNVIELG |
| | | | GKIETLV | LEKVEIDHTP | | | | VARMQTD |
| | | | VDNGA | LDLILIDDELF | | | | VSSDVYLPA |
| | | | EFWSES | VPFGRPYLTL | | | | W* (SEQ ID |
| | | | LAQACL | LIDVFSSCIVG | | | | NO: 119) |
| | | | ESGINIQ | FHLGYKAPSY | | | | |
| | | | FNPVRK | DSVSKAIIHA | | | | |
| | | | PWLKPF | TKPKDYLDSI | | | | |
| | | | VERLFG | ASDFQHDW | | | | |
| | | | TINQKF | PCCGKIETLV | | | | |
| | | | LDPFPG | VDNGAEFW | | | | |
| | | | KTFSSVL | SESLAQACLE | | | | |
| | | | EKEEYN | SGINIQFNPV | | | | |
| | | | PEKDAV | RKPWLKPFV | | | | |
| | | | IRFSTFIE | ERLFGTINQK | | | | |
| | | | LFHRWI | FLDPFPGKTF | | | | |
| | | | VDVYH | SSVLEKEEYN | | | | |
| | | | HDADS | PEKDAVIRFS | | | | |
| | | | RKTRIP | TFIELFHRWI | | | | |
| | | | AKLWQ | VDVYHHDA | | | | |
| | | | QGYEDY | DSRKTRIPAK | | | | |
| | | | PPLAMS | LWQQGYED | | | | |
| | | | QEDIDK | YPPLAMSQE | | | | |
| | | | LTVVM | DIDKLTVVM | | | | |
| | | | GVKWQ | GVKWQPTLT | | | | |
| | | | PTLTRL | RLGFKIKHLR | | | | |
| | | | GFKIKH | YDCPELSEYR | | | | |
| | | | LRYDCP | KRYPQTESSR | | | | |
| | | | ELSEYRK | KKLVKIDPDD | | | | |
| | | | RYPQTE | ISRIFVYLEEL | | | | |
| | | | SSRKKL | DGYLEVPCE | | | | |
| | | | VKIDPD | DPIGYTKNLS | | | | |
| | | | DISRIFV | WHQHQVLA | | | | |
| | | | YLEELD | HSHHKFIEGS | | | | |
| | | | GYLEVP | IDVLSLAKAR | | | | |
| | | | CEDPIG | LAIHQRVQQ | | | | |
| | | | YTKNLS | EQEEYRLLPS | | | | |
| | | | WHQH | KVKRERGQR | | | | |
| | | | QVLAHS | KLAEFSGVE | | | | |
| | | | HHKFIE | QGGNSTVAL | | | | |
| | | | GSIDVLS | PSKAAKKDS | | | | |
| | | | LAKARL | KDEGVKGLL | | | | |
| | | | AIHQRV | DDWDDMIS | | | | |
| | | | QQEQE | NLDGY* | | | | |
| | | | EYRLLPS | (SEQ ID | | | | |
| | | | KVKRER | NO: 115) | | | | |
| | | | GQRKLA | | | | | |
| | | | EFSGVE | | | | | |
| | | | QGGNS | | | | | |
| | | | TVALPS | | | | | |
| | | | KAAKKD | | | | | |
| | | | SKDEGV | | | | | |
| | | | KGLLDD | | | | | |
| | | | WDDMI | | | | | |
| | | | SNLDGY | | | | | |
| | | | * (SEQ | | | | | |
| | | | ID | | | | | |
| | | | NO: 114) | | | | | |
| 19 | Vibrio | MSALPS | MTKKSF | MSALPSLST | MDEDRETRI | MDEDRETR | MLLQRPKPH | MPKKKRKV |
| | chagasii | LSTATLI | SSFHRK | ATLIALESAF | SKAKRAFVST | ISKAKRAFV | SNESLESFFIR | GSGEQKLIS |
| | strain | ALESAF | SVLHQE | DTPARSLTKS | PSVTKILGYM | STPSVTKIL | VANKNGYED | EEDLEQKLI |
| | ECSMB | DTPARS | KLEQND | RGKNIHRYV | DRCRELSDFE | GYMDRCRE | VNRFLMATK | SEEDLEQKL |
| | 14107 | LTKSRG | RVVDIN | SAKMGKRVT | SEPTCMMV | LSDFESEPT | RYLQDIDFSG | ISEEDLGSG |
| | | KNIHRY | DVAEAT | VESFLECAAC | FGASGVGKT | CMMVFGA | FQTFPTNICK | LLQRPKPHS |
| | | VSAKM | YKDISAF | YHFDFEPSIV | TIIKKYLSQN | SGVGKTTII | INPASAKSSS | NESLESFFIR |
| | | GKRVTV | PEKIVVE | RFCSQPIRLS | KRDSEARGD | KKYLSQNK | SARIASLLKL | VANKNGYE |
| | | ESFLEC | ITFRLSIL | YCLNGKTHT | VVPVLHIELP | RDSEARGD | AQLTFNEPP | DVNRFLMA |
| | | AACYHF | RLLGRK | YVPDFLVQF | DNAKPVDAA | VVPVLHIEL | DLLGLAINRT | TKRYLQDID |
| | | DFEPSIV | CEKIVPK | DTGDYKLYE | RELLLEMRD | PDNAKPVD | NLKYSPSTSA | FSGFQTFPT |
| | | RFCSQP | SIEPHR | VKSDMESSK | PLALYETDLA | AARELLLE | VIRGSEVFPR | NICKINPAS |
| | | IRLSYCL | VDLQRS | EEFHCEWEA | RLTKRLTDLI | MRDPLALY | SLLRTKSIPCC | AKSSSSARI |
| | | NGKTHT | HDRKIP | KVQGAFGIG | PVTGVKLIIID | ETDLARLTK | PLCLQQNDY | ASLLKLAQL |
| | | YVPDFL | SAITIYR | LDLELVTEEEI | EFQHLVEERS | RLTDLIPVT | ASYLWHFEG | TFNEPPDLL |
| | | VQFDT | WWLTF | -NEVIFSNLK | NRVLTQVGN | GVKLIIIDEF | YDHCHIHDA | GLAINRTNL |
| | | GDYKLY | RESDYN | LLHRYASRD | WLKMILNRT | QHLVEERS | PLLNSCRCG | KYSPSTSAV |
| | | EVKSD | PVSLAP | HLNDFHQTL | KCPIVLFGM | NRVLTQVG | AEYDYRVSG | IRGSEVFPR |
| | | MESSKE | DFKSRG | LATLKPNGT | PYSKVVLKA | NWLKMILN | LSGMCGECK | SLLRTKSIPC |
| | | EFHCE | NRDPKV | QTARSLGHH | NSQLHGRFSI | RTKCPIVLF | KTISTKSSEN | CPLCLQQN |
| | | WEAKV | APIVDAI | LGLSGRKILPI | QFELRPFNY | GMPYSKVV | SHKATSTVSS | DYASYLWH |
| | | QGAFGI | MKQAV | LCDLLSRNLL | QNGEGVFKT | LKANSQLH | WLAGNESK | FEGYDHCHI |
| | | GLDLEL | ESVISGR | QTNLETPLSL | FLEHLDKALP | GRFSIQFEL | DLPDVPKSY | HDAPLLNS |
| | | VTEEEIL | KININSA | ESEFELVCYD | FEKEVGLVE | RPFNYQNG | RWGLIHWW | CRCGAEYD |
| | | NEVIFS | YRRVKR | GSGPKKKRK | QGLQKKLYA | EGVFKTFLE | VHISKNEFD | YRVSGLSG |
| | | NLKLLH | KVRQY | VGSGYPYDV | FSQGNMRSL | HLDKALPFE | HVSFIQFFSK | MCGECKKT |
| | | RYASRD | NLTHST | PDYAYPYDV | RNLIYQASVE | KEVGLVEQ | WPSSFHSMI | ISTKSSENS |
| | | HLNDFH | KYKYPE | PDYAYPYDV | AIDKQHETIT | GLQKKLYAF | DNEIEFNLEH | HKATSTVSS |
| | | QTLLAT | YESVRIR | PDYAGSGTK | EQDLIFASKL | SQGNMRSL | AIVGRRELRI | WLAGNESK |
| | | LKPNGT | VKKKTP | KSFSSFHRKS | TSGDKSDRW | RNLIYQASV | KDLLGRIFFS | DLPDVPKSY |
| | | QTARSL | FEILAAK | VLHQEKLEQ | ENPFEKGVK | EAIDKQHET | SVRLPERNL | RWGLIHW |
| | | GHHLGL | KGERVA | NDRVVDIND | VTEGMLRSP | ITEQDLIFAS | QHNIVLGELL | WVHISKNE |
| | | SGRKILP | KREFRR | VAEATYKDIS | PKDIGWEDY | KLTSGDKSD | RHTEMHLW | FDHVSFIQF |
| | | ILCDLLS | MGRKIL | AFPEKIVVEIT | YHHVTSLNA | RWENPFEK | DNNGLIANL | FSKWPSSF |
| | | RNLLQT | TSSVLE | FRLSILRLLGR | KRNGGNMF | GVKVTEGM | RMNALETTV | HSMIDNEIE |
| | | NLETPL | RVEIDH | KCEKIVPKSIE | E* (SEQ ID | LRSPPKDIG | FLNCSKDELA | FNLEHAIVG |
| | | SLESEFE | TVLDLF | PHRVDLQRS | NO: 123) | SGPAAKKK | SMVEQRILK | RRELRIKDLL |
| | | LVCYD | AVHEEH | HDRKIPSAITI | | KLDGSGG | PNRKTKPN | GRIFFSSVR |
| | | (SEQ ID | RIPLGR | YRWWLTFRE | | WEDYYHH | MPLAVNDYL | LPERNLQH |
| | | NO: 120) | PWLTQL | SDYNPVSLA | | VTSLNAKR | FYFGDIFCLW | NIVLGELLR |
| | | | VDCYSK | PDFKSRGNR | | NGGNMFE | LAEFQTDEF | HTEMHLW |
| | | | AVIGFYL | DPKVAPIVD | | * (SEQ ID | NRSFYVSRW | DNNGLIAN |
| | | | GFEPPS | AIMKQAVES | | NO: 124) | * (SEQ ID | LRMNALET |
| | | | YMSVSL | VISGRKININ | | | NO: 125) | TVFLNCSKD |
| | | | ALKNAI | SAYRRVKRK | | | | ELASMVEQ |
| | | | QRKDTL | VRQYNLTHS | | | | RILKPNRKT |
| | | | LSSYPSI | TKYKYPEYES | | | | KPNMPLAV |
| | | | ENEWL | VRIRVKKKTP | | | | NDYLFYFG |
| | | | CYGIPD | FEILAAKKGE | | | | DIFCLWLAE |
| | | | LLVTDN | RVAKREFRR | | | | FQTDEFNR |
| | | | GKEFLS | MGRKILTSSV | | | | SFYVSRW* |
| | | | KAFDKA | LERVEIDHTV | | | | (SEQ ID |
| | | | CESLLIN | LDLFAVHEE | | | | NO: 126) |
| | | | VHQNK | HRIPLGRPW | | | | |
| | | | VETPDN | LTQLVDCYSK | | | | |
| | | | KPHVER | AVIGFYLGFE | | | | |
| | | | NYGTIN | PPSYMSVSL | | | | |
| | | | TSLLDD | ALKNAIQRK | | | | |
| | | | LPGKAF | DTLLSSYPSIE | | | | |
| | | | SQYLQR | NEWLCYGIP | | | | |
| | | | EGYDSV | DLLVTDNGK | | | | |
| | | | SEATLTL | EFLSKAFDKA | | | | |
| | | | DEIKEIY | CESLLINVHQ | | | | |
| | | | LIWLVDI | NKVETPDNK | | | | |
| | | | YHRKPN | PHVERNYGT | | | | |
| | | | QRGTN | INTSLLDDLP | | | | |
| | | | CPNVA | GKAFSQYLQ | | | | |
| | | | WRQGC | REGYDSVSE | | | | |
| | | | QNWEP | ATLTLDEIKEI | | | | |
| | | | EEFLGS | YLIWLVDIYH | | | | |
| | | | KDELDF | RKPNQRGTN | | | | |
| | | | KFAIED | CPNVAWRQ | | | | |
| | | | HKQLTK | GCQNWEPE | | | | |
| | | | AGITVS | EFLGSKDELD | | | | |
| | | | KGLTYS | FKFAIEDHKQ | | | | |
| | | | SERLAG | LTKAGITVSK | | | | |
| | | | YMGKK | GLTYSSERLA | | | | |
| | | | GNHKV | GYMGKKGN | | | | |
| | | | QFKYNP | HKVQFKYNP | | | | |
| | | | ECMAVI | ECMAVIWVL | | | | |
| | | | WVLDE | DEDVNEYFT | | | | |
| | | | DVNEYF | VNAIDYESAR | | | | |
| | | | TVNAID | RVSLWQHKY | | | | |
| | | | YESARR | NMKYQAEL | | | | |
| | | | VSLWQ | NSAEYDEDK | | | | |
| | | | HKYNM | EIDAEIKIEEI | | | | |
| | | | KYQAEL | ADRSILETKKI | | | | |
| | | | NSAEYD | RSRRRGARH | | | | |
| | | | EDKEID | QENSARAKSI | | | | |
| | | | AEIKIEEI | SNTKLVPPQ | | | | |
| | | | ADRSILE | KDEEEIVIVD | | | | |
| | | | TKKIRSR | NEDWDIDYV | | | | |
| | | | RRGAR | * (SEQ ID | | | | |
| | | | HQENS | NO: 122) | | | | |
| | | | ARAKSIS | | | | | |
| | | | NTKLVP | | | | | |
| | | | PQKDEE | | | | | |
| | | | EIVIVDN | | | | | |
| | | | EDWDI | | | | | |
| | | | DYV* | | | | | |
| | | | (SEQ ID | | | | | |
| | | | NO: 121) | | | | | |
| 20 | Vibrio | MLCQY | MPKKSF | MLCQYDSFS | MDDSRDIRI | MDDSRDIRI | MLLQRPKTY | MPKKKRKV |
| | roti- | DSFSEDI | SNFNRK | EDITLALDNA | ARAKKAFVIT | ARAKKAFVI | PDESLESFFIR | GSGEQKLIS |
| | ferianus | TLALDN | AKLEVS | FHNPARKLT | PSVAKVLRY | TPSVAKVLR | VANKNGYD | EEDLEQKLI |
| | CAIM 577 | AFHNPA | DYQEDL | KSRGKNIHR | MDRCRDFS | YMDRCRDF | DIQRFLEALK | SEEDLEQKL |
| | APHW0100 | RKLTKS | VDIDSSL | YASAKMGKR | DMDSEPTC | SDMDSEPT | RFLIDKNPRQ | ISEEDLGSG |
| | 0105 | RGKNIH | NDALAE | VTVESALECD | MIVYGASGV | CMIVYGAS | FQTFPTNICK | LLQRPKTYP |
| | | RYASAK | DITYKDL | ACYHFDFEK | GKTTIIKKYLK | GVGKTTIIK | INPYSSKNHS | DESLESFFIR |
| | | MGKRV | TAFPDK | DIIRFCSQPIR | KNEGDSDID | KYLKKNEG | ISRTNALLELS | VANKNGYD |
| | | TVESAL | VANEIS | YSYYYNGKW | GDTIPVVHIE | DSDIDGDTI | HMTFNEPA | DIQRFLEAL |
| | | ECDACY | YRLKVL | HTYVPDFLV | LPDNAKPVD | PVVHIELPD | NLLGMALNR | KRFLIDKNP |
| | | HFDFEK | KYLGKE | QFDTGEYVL | AARELLLKM | NAKPVDAA | NQMKFSPST | RQFQTFPT |
| | | DIIRFCS | CDKITP | YEIKPDDIAS | GDPLALYDT | RELLLKMG | TALIRGAEVI | NICKINPYS |
| | | QPIRYSY | KTIEPH | SPDFLDEWS | DLARLTKRIV | DPLALYDTD | PRSLLLKDSV | SKNHSISRT |
| | | YYNGK | RVELQR | AKQQAAEE | ELIPALGVKL | LARLTKRIV | PCCPMCLHE | NALLELSH |
| | | WHTYV | CNDKKI | MGLELELVE | IIDEFQHLVE | ELIPALGVK | KGYANYRW | MTFNEPAN |
| | | PDFLVQ | PSAITIY | EKQIRNKTLL | ESSNKILTQV | LIIIDEFQHL | HFSGYDYCH | LLGMALNR |
| | | FDTGEY | RWWLN | KNLKLMYRY | GNWLKGILN | VEESSNKIL | EHNVKLVSH | NQMKFSPS |
| | | VLYEIKP | FSQSDF | ASRDCLTDT | KSKCPIVLFG | TQVGNWL | CTCGSTYDY | TTALIRGAE |
| | | DDIASS | NPTCLA | HNLVLNILRD | MPYSKLVLQ | KGILNKSKC | RTAGLSGICP | VIPRSLLLKD |
| | | PDFLDE | PDFKGR | NGPQSAQH | ANSQLHGRF | PIVLFGMPY | ECGDIIASAQ | SVPCCPMC |
| | | WSAKQ | GNREPK | LIHKAGLTRR | SIQFDLRPFS | SKLVLQANS | VHDDSSGVK | LHEKGYAN |
| | | QAAEE | VPKIVD | AIMPVLCNLL | YQEGEGTFK | QLHGRFSIQ | IASWLSGFD | YRWHFSGY |
| | | MGLELE | ALMEQ | SRNLLETELD | TFLQHLDEAL | FDLRPFSYQ | VDPLPIIPQS | DYCHEHNV |
| | | LVEEKQI | AVEGVI | SPLSLKSEFK | PFEKQAGLA | EGEGTFKTF | YRWGLIHW | KLVSHCTC |
| | | RNKTLL | SGKKINI | VNCYAGSGP | NEGLQKKLY | LQHLDEALP | WSQMFGAT | GSTYDYRT |
| | | KNLKLM | SSAYRR | KKKRKVGSG | AFSQGNMR | FEKQAGLA | QTSDSEKFVT | AGLSGICPE |
| | | YRYASR | VRRKVR | YPYDVPDYA | SLRDLIYHASI | NEGLQKKL | FWEQWPNS | CGDIIASAQ |
| | | DCLTDT | QYNVK | YPYDVPDYA | EAIDNHHESI | YAFSQGN | FHDMIETEIE | VHDDSSGV |
| | | HNLVLN | NGTKH | YPYDVPDYA | TKDDFLFAS | MRSLRDLIY | TGFEYAVVS | KIASWLSGF |
| | | ILRDNG | KYPKYE | GSGPKKSFS | QLTSGNKST | HASIEAIDN | HTELRIKNVL | DVDPLPIIP |
| | | PQSAQ | SLRKRV | NFNRKAKLE | FWKNPFIEG | HHESITKDD | GKILFSSIKLP | QSYRWGLI |
| | | HLIHKA | NKKTPF | VSDYQEDLV | VKVTKDMLR | FLFASQLTS | DRNFRSNIIL | HWWSQM |
| | | GLTRRA | EILSAKK | DIDSSLNDAL | SPPKSIGWE | GNKSTFWK | KELFQYLEAH | FGATQTSD |
| | | IMPVLC | GVRVAK | AEDITYKDLT | DYYQQNNS | NPFIEGVKV | LWDNDGRL | SEKFVTFW |
| | | NLLSRN | REFRKM | AFPDKVANE | RKKKGKGRP | TKDMLRSP | ANLRLNTSDI | EQWPNSFH |
| | | LLETELD | GKKILTS | ISYRLKVLKYL | DFFD* (SEQ | PKSIGSGPA | CIVLNCSKEQ | DMIETEIET |
| | | SPLSLKS | YALERV | GKECDKITPK | ID NO: 130) | AKKKKLDG | VASMVEQRI | GFEYAVVS |
| | | EFKVNC | EVDHTV | TIEPHRVELQ | | SGGWEDYY | LIPTRHPKSR | HTELRIKNV |
| | | YA* | LDVFVV | RCNDKKIPSA | | QQNNSRKK | GILIDTNYVY | LGKILFSSIK |
| | | (SEQ ID | HEEYRIP | ITIYRWWLN | | KGKGRPDF | YFGDIYCLWL | LPDRNFRS |
| | | NO: 127) | LGRPYL | FSQSDFNPT | | FD* (SEQ | SEFQTDEFN | NIILKELFQY |
| | | | TQLVDC | CLAPDFKGR | | ID NO: 131) | RSFYVSRW* | LEAHLWDN |
| | | | YSKAVV | GNREPKVPKI | | | (SEQ ID | DGRLANLR |
| | | | GFYLGF | VDALMEQA | | | NO: 132) | LNTSDICIVL |
| | | | EPPSYV | VEGVISGKKI | | | | NCSKEQVA |
| | | | SVSLAL | NISSAYRRVR | | | | SMVEQRILI |
| | | | KNAIQR | RKVRQYNVK | | | | PTRHPKSR |
| | | | KDSLLSS | NGTKHKYPK | | | | GILIDTNYV |
| | | | YPSVKN | YESLRKRVNK | | | | YYFGDIYCL |
| | | | EWLCY | KTPFEILSAK | | | | WLSEFQTD |
| | | | GIMDLL | KGVRVAKRE | | | | EFNRSFYVS |
| | | | VTDNG | FRKMGKKILT | | | | RW* (SEQ |
| | | | KEFLSK | SYALERVEVD | | | | ID NO: 133) |
| | | | AFDAAC | HTVLDVFVV | | | | |
| | | | ETLLITV | HEEYRIPLGR | | | | |
| | | | HQNKV | PYLTQLVDCY | | | | |
| | | | ETPDNK | SKAVVGFYL | | | | |
| | | | PHVERN | GFEPPSYVSV | | | | |
| | | | YGTVNT | SLALKNAIQR | | | | |
| | | | NVLDDL | KDSLLSSYPS | | | | |
| | | | PGKAFS | VKNEWLCYG | | | | |
| | | | HYIQRE | IMDLLVTDN | | | | |
| | | | GYDSIG | GKEFLSKAFD | | | | |
| | | | EATLTLS | AACETLLITV | | | | |
| | | | ELKEVYL | HQNKVETPD | | | | |
| | | | IWLVDK | NKPHVERNY | | | | |
| | | | YHRKPN | GTVNTNVLD | | | | |
| | | | QRGTN | DLPGKAFSH | | | | |
| | | | CPNVA | YIQREGYDSI | | | | |
| | | | WKRGC | GEATLTLSEL | | | | |
| | | | EEWEPE | KEVYLIWLV | | | | |
| | | | EFTGTA | DKYHRKPNQ | | | | |
| | | | AELDFK | RGTNCPNVA | | | | |
| | | | FAILDKK | WKRGCEEW | | | | |
| | | | KLNKSG | EPEEFTGTAA | | | | |
| | | | ITVYVDL | ELDFKFAILD | | | | |
| | | | TYTSDR | KKKLNKSGIT | | | | |
| | | | LAEYRG | VYVDLTYTSD | | | | |
| | | | RKGNH | RLAEYRGRK | | | | |
| | | | VVTFKY | GNHVVTFKY | | | | |
| | | | NPECM | NPECMGHI | | | | |
| | | | GHIWVL | WVLDEDAN | | | | |
| | | | DEDAN | EYFTVPAIDY | | | | |
| | | | EYFTVP | EYASSISLWQ | | | | |
| | | | AIDYEY | HKFNIKYQR | | | | |
| | | | ASSISL | NLNSADYDE | | | | |
| | | | WQHKF | DAEIDAEIR | | | | |
| | | | NIKYQR | MEEVAEESI | | | | |
| | | | NLNSAD | VKTKKIRNRR | | | | |
| | | | YDEDAE | RGARYQENT | | | | |
| | | | IDAEIR | ERAKSQNQK | | | | |
| | | | MEEVA | SLEKAEQGH | | | | |
| | | | EESIVKT | HQEEDVYDE | | | | |
| | | | KKIRNR | NAWGIDYL* | | | | |
| | | | RRGARY | (SEQ ID | | | | |
| | | | QENTER | NO: 129) | | | | |
| | | | AKSQN | | | | | |
| | | | QKSLEK | | | | | |
| | | | AEQGH | | | | | |
| | | | HQEED | | | | | |
| | | | VYDENA | | | | | |
| | | | WGIDYL | | | | | |
| | | | * (SEQ | | | | | |
| | | | ID | | | | | |
| | | | NO: 128) | | | | | |
| 21 | 1004634 | MKKRII | MSDDS | MKKRIIKNSK | VNHFARAPH | MNHFARA | MMLLQRPK | MPKKKRKV |
| | 327 | KNSKVK | ENLYAF | VKNISRFVSL | QQVKSIFISN | PHQQVKSIF | SYPDESLESF | GSGEQKLIS |
| | RIMD- | NISRFVS | GSFFPE | KTDSVQTTE | SQIDEILSDIE | ISNSQIDEIL | FIRVANKNG | EEDLEQKLI |
| | BA000032. | LKTDSV | KHSNTS | SDLEFDACH | ECREESDGIS | SDIEECREE | YNDVHWFL | SEEDLEQKL |
| | 2 | QTTESD | VPKTSK | HFEFASHVK | EPECLIVVGD | SDGISEPEC | VAVKRYLLDI | ISEEDLGSG |
| | | LEFDAC | GTRFGI | SFETQPLGFE | SGSGKTTIID | LIVVGDSGS | DPRKFQTFP | MLLQRPKS |
| | | FHFEFA | ELQESY | YRLNGRLRR | KYLVDNPRM | GKTTIIDKYL | TDICCINPYS | YPDESLESF |
| | | SHVKSF | QDLFSF | YTPDMLCYF | EANDGSIIPIL | VDNPRME | SKKHSISRTH | FIRVANKN |
| | | ETQPLG | DEKRRD | NDGYATYYE | FTSLPANAN | ANDGSIIPIL | ALHHLSQLTF | GYNDVHW |
| | | FEYRLN | EAIHRY | VKPKWVTER | PVTASERLLS | FTSLPANA | NEPVDLLGIA | FLVAVKRYL |
| | | GRLRRY | NILDYLI | DEFKKKFDA | SMGDPLAFS | NPVTASERL | LNRNQMQF | LDIDPRKFQ |
| | | TPDML | ELHGPS | QKQQAIAN | HGKDPAEL | LSSMGDPL | SPSTTALIRG | TFPTDICCI |
| | | CYFNDG | LTLKKIS | GYDLLVLTED | MKIVNDLLR | AFSHGKDP | AEVIPRSLLR | NPYSSKKHS |
| | | YATYYE | GSMKG | DIQTYPLLDN | ECRVELIIIDE | AELMKIVN | KGAIPCCPCC | ISRTHALHH |
| | | VKPKW | LADKFH | LKIIHRYACS | FQHMIDRKS | DLLRECRVE | LGEHGYASY | LSQLTFNEP |
| | | VTERDE | PNVPSA | DSLDDVQVR | KDVLHSTAD | LIIIDEFQH | RWHFSGYEY | VDLLGIALN |
| | | FKKKFD | PSIYRY | ILKLFQNYGE | WLKMIIIDSK | MIDRKSKD | CHEHDVKLIE | RNQMQFS |
| | | AQKQQ | WTTFKK | MRISQVINA | IPVVLFGMP | VLHSTADW | RCSCGAIYDY | PSTTALIRG |
| | | AIANGY | SGFVLS | SQGQSASILP | YSTEILRVNN | LKMIIIDSKI | RYAGLSGVC | AEVIPRSLL |
| | | DLLVLTE | SLIPGVT | ALYDLIAKKIL | QLRGRFESQ | PVVLFGMP | TECGENISAS | RKGAIPCCP |
| | | DDIQTY | RGNTK | EFDWHCPIS | HHLKPFRVK | YSTEILRVN | QENHEPKAT | CCLGEHGY |
| | | PLLDNL | QRKTLE | HDSLVWRVS | DTSELIRYKTF | NQLRGRFE | RIASWLAGD | ASYRWHFS |
| | | KIIHRYA | LEEYIER | GSGPKKKRK | MTMLDAAL | SQHHLKPF | DVKPLPDVP | GYEYCHEH |
| | | CSDSLD | AIKSYFS | VGSGYPYDV | PFLEESGLAS | RVKDTSEL | LSYRWGFM | DVKLIERCS |
| | | DVQVRI | AESPTI | PDYAYPYDV | EDIMKRVYIF | RYKTFMTM | HWWSQISSS | CGAIYDYRY |
| | | LKLFQN | QQAFTL | PDYAYPYDV | SKGNMRLIR | LDAALPFLE | CKTRNNGEF | AGLSGVCT |
| | | YGEMRI | LETEIDR | PDYAGSGSD | RLINKAAKFA | ESGLASEDI | LAFWEHWP | ECGENISAS |
| | | SQVINA | HNECN | DSENLYAFG | LLENAPCISL | MKRVYIFSK | NSFHKLIGKE | QENHEPKA |
| | | SQGQS | DTQLSF | SFFPEKHSNT | KHFARAAPK | GNMRLIRR | IDFNFEYCVL | TRIASWLA |
| | | ASILPAL | EYESFR | SVPKTSKGTR | VSRDACKSF | LINKAAKFA | SKNDLRVKDI | GDDVKPLP |
| | | YDLIAKK | KRIVKKT | FGIELQESYQ | NPFDTDTKK | LLENAPCISL | LGKILFSSIQL | DVPLSYRW |
| | | ILEFDW | DYERLLI | DLFSFDEKRR | LKIIEPPEDV | KHFARAAP | PDRNFRSNII | GFMHWW |
| | | HCPISH | KKGKKA | DEAIHRYNIL | GWENYLAA | KVSRDACK | LKEMFQYIET | SQISSSCKT |
| | | DSLVW | ADTYYK | DYLIELHGPS | KGD (SEQ ID | SFNPFDTDT | HLWDDNGK | RNNGEFLA |
| | | RVS | KVGQR | LTLKKISGSM | NO: 137) | KKLKIIEPPE | LANLRMNM | FWEHWPN |
| | | (SEQ ID | PETTRV | KGLADKFHP | | DVGSGPAA | LEICVLLNCS | SFHKLIGKEI |
| | | NO: 134) | LQRVEA | NVPSAPSIYR | | KKKKLDGS | REQVTSMIE | DFNFEYCVL |
| | | | DHTRLD | YWTTFKKSG | | GGWENYL | QGLLPPNRQ | SKNDLRVK |
| | | | LFVIDD | FVLSSLIPGV | | AAKGD* | LGKREILIVTE | DILGKILFSSI |
| | | | ARKLPL | TRGNTKQRK | | (SEQ ID | YAFYLGDVY | QLPDRNFR |
| | | | GRPWL | TLELEEYIERA | | NO: 138) | CLWLSEFQS | SNIILKEMF |
| | | | TLLFDT | IKSYFSAESPT | | | DEFNRSFYLS | QYIETHLW |
| | | | HTKSVV | IQQAFTLLET | | | RW (SEQ ID | DDNGKLAN |
| | | | GFYLGF | EIDRHNECN | | | NO: 139) | LRMNMLEI |
| | | | EPPGYL | DTQLSFEYES | | | | CVLLNCSRE |
| | | | SVSLALE | FRKRIVKKTD | | | | QVTSMIEQ |
| | | | NAILPKY | YERLLIKKGK | | | | GLLPPNRQ |
| | | | YVKELY | KAADTYYKK | | | | LGKREILIVT |
| | | | PEVKGE | VGQRPETTR | | | | EYAFYLGDV |
| | | | WPCYG | VLQRVEADH | | | | YCLWLSEF |
| | | | LPEHLIV | TRLDLFVIDD | | | | QSDEFNRS |
| | | | DNGAEF | ARKLPLGRP | | | | FYLSRW |
| | | | NSKDFV | WLTLLFDTH | | | | (SEQ ID |
| | | | TACKNL | TKSVVGFYL | | | | NO: 140) |
| | | | RIKVKK | GFEPPGYLSV | | | | |
| | | | NPVKKP | SLALENAILP | | | | |
| | | | WLKGS | KYYVKELYPE | | | | |
| | | | VERYFR | VKGEWPCY | | | | |
| | | | TINNKLL | GLPEHLIVDN | | | | |
| | | | SGIPGK | GAEFNSKDF | | | | |
| | | | SFSNIFA | VTACKNLRIK | | | | |
| | | | RGDYN | VKKNPVKKP | | | | |
| | | | PQKNAI | WLKGSVERY | | | | |
| | | | ITRSDL | FRTINNKLLS | | | | |
| | | | MKVIHV | GIPGKSFSNI | | | | |
| | | | WLIDIY | FARGDYNPQ | | | | |
| | | | QSSPNG | KNAIITRSDL | | | | |
| | | | LENNIP | MKVIHVWLI | | | | |
| | | | NLSWA | DIYQSSPNGL | | | | |
| | | | DAMRS | ENNIPNLSW | | | | |
| | | | AFPPRS | ADAMRSAFP | | | | |
| | | | FNGSID | PRSFNGSIDE | | | | |
| | | | ELRFNL | LRFNLGKHV | | | | |
| | | | GKHVEI | EISLDRNGIR | | | | |
| | | | SLDRNG | LKKTLRYTSS | | | | |
| | | | IRLKKTL | YLAQYFGKH | | | | |
| | | | RYTSSYL | TYDGKSIKVK | | | | |
| | | | AQYFGK | IKYNPICMGS | | | | |
| | | | HTYDGK | IYVLDEDKHE | | | | |
| | | | SIKVKIK | FFAVESVDP | | | | |
| | | | YNPICM | DYAYSVSEW | | | | |
| | | | GSIYVL | LHKVCCDYA | | | | |
| | | | DEDKHE | RNHIRNNYR | | | | |
| | | | FFAVES | HNDVIKAW | | | | |
| | | | VDPDYA | RVIYDIIDEAL | | | | |
| | | | YSVSEW | HLSGNGKQA | | | | |
| | | | LHKVCC | NVGIRQASK | | | | |
| | | | DYARN | LERVREHAE | | | | |
| | | | HIRNNY | RTKSHQKPE | | | | |
| | | | RHNDVI | LHMSSNDDI | | | | |
| | | | KAWRVI | DWDVEVNT | | | | |
| | | | YDIIDEA | DGWKIDSVR | | | | |
| | | | LHLSGN | GTNK* (SEQ | | | | |
| | | | GKQAN | ID NO: 136) | | | | |
| | | | VGIRQA | | | | | |
| | | | SKLERV | | | | | |
| | | | REHAER | | | | | |
| | | | TKSHQK | | | | | |
| | | | PELHMS | | | | | |
| | | | SNDDID | | | | | |
| | | | WDVEV | | | | | |
| | | | NTDGW | | | | | |
| | | | KIDSVR | | | | | |
| | | | GTNK | | | | | |
| | | | (SEQ ID | | | | | |
| | | | NO: 135) | | | | | |
| 22 | V. | MLCQD | MAKKSF | MLCQDSFSE | MDDSRDIRV | MDDSRDIR | MLLQRPKPY | MPKKKRKV |
| | para_O1 | SFSENV | SNFNRK | NVVLALEQA | AKAKKAFVIT | VAKAKKAF | PDESLESFFIR | GSGEQKLIS |
| | Kuk FDA | VLALEQ | AKRDD | FHNPARKLT | PSVAKVLRY | VITPSVAKV | VANKNGYSD | EEDLEQKLI |
| | R31 GCA | AFHNPA | VSHQEE | KSRGKNIHRF | MDRCRDLSD | LRYMDRCR | VNWFLLAVK | SEEDLEQKL |
| | 0004304 | RKLTKS | TLYIDRA | ASAKMGKR | MDSEPTCM | DLSDMDSE | RYLLGIDPRK | ISEEDLGSG |
| | 051 | RGKNIH | LNDTLD | VTVESALECD | MVYGSHGV | PTCMMVY | FQTFPTDICR | LLQRPKPYP |
| | | RFASAK | EDATYT | ACYCFDFEK | GKTAIIKKYLK | GSHGVGKT | INPHSSKKHS | DESLESFFIR |
| | | MGKRV | DLTAFP | DIIRFCAQPIR | QNEGDSDTE | AIIKKYLKQ | ISRTHALHHL | VANKNGYS |
| | | TVESAL | DKVAIEI | YSYYYNGKW | GDTIPVIHIE | NEGDSDTE | SQLTFNEPV | DVNWFLLA |
| | | ECDACY | SFRLKIL | RTYVPDFLV | MPDNAKPV | GDTIPVIHIE | DLLGIALNRN | VKRYLLGID |
| | | CFDFEK | RYLGRV | QFDTGEYVL | DAARELLLQ | MPDNAKP | QMQFSTSTT | PRKFQTFPT |
| | | DIIRFCA | NDKIVP | YEVKPDNIAS | MEDPLALYD | VDAARELLL | AVIRGAEVIP | DICRINPHS |
| | | QPIRYSY | KTIEPH | SSDFLDEWN | TDLARLTKRI | QMEDPLAL | RSLLRKGVIP | SKKHSISRT |
| | | YYNGK | RVTLQR | AKQQAAQT | VELIPLLGVK | YDTDLARLT | CCPSCLGEH | HALHHLSQ |
| | | WRTYV | CNDKNI | RGLELELVEE | LIIIDEFQHLV | KRIVELIPLL | GYASYRWHF | LTFNEPVDL |
| | | PDFLVQ | PSAITIY | KQIRVKNLLK | DESSNKILTQ | GVKLIIIDEF | SGYEYCHEH | LGIALNRN |
| | | FDTGEY | RWWLN | NLKLMHRYA | VGNWLKGIL | QHLVDESS | DVKLIERCSC | QMQFSTST |
| | | VLYEVK | FSQSGY | SRDCLSDKH | NKSKCPIVLF | NKILTQVG | GAVYDYRYA | TAVIRGAEV |
| | | PDNIAS | NPTSLA | NLVLNILRKN | GMPYSKLVL | NWLKGILN | GLSGVCTEC | IPRSLLRKG |
| | | SSDFLD | PKFKGR | GSQSAQYLS | QANSQLHSR | KSKCPIVLF | GENISASQE | VIPCCPSCL |
| | | EWNAK | GNRAP | DKTGLSRRAI | FSIQFNLRPF | GMPYSKLV | NHEPKATRI | GEHGYASY |
| | | QQAAQ | KVSEIV | MPVLCNLLS | NYQEGEGTF | LQANSQLH | ASWLAGDD | RWHFSGYE |
| | | TRGLEL | DALMA | RNLLETDLDT | KTFLQHLDE | SRFSIQFNL | VKPLPDVPLS | YCHEHDVK |
| | | ELVEEK | QAVEA | PISFQSEFEL | ALPFEKQTGL | RPFNYQEG | YRWGFMH | LIERCSCGA |
| | | QIRVKN | VISGRK | VSYGGSGPK | AKEGLQEKL | EGTFKTFLQ | WWSQISSSC | VYDYRYAG |
| | | LLKNLKL | NVSSAH | KKRKVGSGY | YAFSQGNM | HLDEALPFE | KTRNNGEFL | LSGVCTECG |
| | | MHRYA | RRVRRK | PYDVPDYAY | RSLRDLIYQA | KQTGLAKE | AFWEHWPN | ENISASQEN |
| | | SRDCLS | VRQYNL | PYDVPDYAY | SIEAIDNHHE | GLQEKLYAF | SFHKLIGKEI | HEPKATRIA |
| | | DKHNLV | KHGTKY | PYDVPDYAG | SITKDDFLFA | SQGNMRSL | DFNFEYCVLS | SWLAGDD |
| | | LNILRK | KYPRYE | SGAKKSFSNF | SQLTSGNKP | RDLIYQASIE | KNDLRVKDIL | VKPLPDVPL |
| | | NGSQS | SVRKRV | NRKAKRDDV | TFWKNPFIE | AIDNHHESI | GKILFSSIQLP | SYRWGFM |
| | | AQYLSD | KKKTPF | SHQEETLYID | GVKVTKEML | TKDDFLFAS | DRNFRSNIIL | HWWSQISS |
| | | KTGLSR | EVLVAK | RALNDTLDE | RSPPRSIGW | QLTSGNKP | KEMFQYIET | SCKTRNNG |
| | | RAIMPV | KGERVA | DATYTDLTAF | EDYYQQNNS | TFWKNPFIE | HLWSDNGR | EFLAFWEH |
| | | LCNLLS | KREFRR | PDKVAIEISF | RKKKGKGRP | GVKVTKEM | LANLRVNTLE | WPNSFHKL |
| | | RNLLET | MGKKIL | RLKILRYLGR | DFFDK (SEQ | LRSPPRSIG | ICVLLNCSRE | IGKEIDFNF |
| | | DLDTPIS | TSYALE | VNDKIVPKTI | ID NO: 144) | SGPAAKKK | QVTSMIEQG | EYCVLSKND |
| | | FQSEFE | RVEVDH | EPHRVTLQR | | KLDGSGG | LLRPNRQLG | LRVKDILGKI |
| | | LVSYG | TVVDLF | CNDKNIPSAI | | WEDYYQQ | KQETLIVTEY | LFSSIQLPD |
| | | (SEQ ID | AVHKEY | TIYRWWLNF | | NNSRKKKG | AFYLGDVYCL | RNFRSNIILK |
| | | NO: 141) | RLPLGR | SQSGYNPTS | | KGRPDFFD | WLSEFQSDE | EMFQYIET |
| | | | PYLTQL | LAPKFKGRG | | K* (SEQ ID | FNRSFYLSR | HLWSDNG |
| | | | VDCYSK | NRAPKVSEIV | | NO: 145) | W (SEQ ID | RLANLRVN |
| | | | AVVGFY | DALMAQAV | | | NO: 146) | TLEICVLLNC |
| | | | LGFEPP | EAVISGRKIN | | | | SREQVTSM |
| | | | SYVSVA | VSSAHRRVR | | | | IEQGLLRPN |
| | | | LALKNAI | RKVRQYNLK | | | | RQLGKQET |
| | | | QRKDSL | HGTKYKYPR | | | | LIVTEYAFYL |
| | | | LSSYPTV | YESVRKRVKK | | | | GDVYCLWL |
| | | | KNEWL | KTPFEVLVAK | | | | SEFQSDEFN |
| | | | CYGIPD | KGERVAKRE | | | | RSFYLSRW |
| | | | LLVTDN | FRRMGKKIL | | | | (SEQ ID |
| | | | GKEFLS | TSYALERVEV | | | | NO: 147) |
| | | | KAFDAA | DHTVVDLFA | | | | |
| | | | CETLLIT | VHKEYRLPLG | | | | |
| | | | VHQNK | RPYLTQLVD | | | | |
| | | | VDTPD | CYSKAVVGF | | | | |
| | | | NKPDVE | YLGFEPPSYV | | | | |
| | | | RKYGTV | SVALALKNAI | | | | |
| | | | NTTLLD | QRKDSLLSSY | | | | |
| | | | DLPGKA | PTVKNEWLC | | | | |
| | | | FSQYLH | YGIPDLLVTD | | | | |
| | | | REGYDS | NGKEFLSKAF | | | | |
| | | | IDEATLT | DAACETLLIT | | | | |
| | | | LDEIKEI | VHQNKVDTP | | | | |
| | | | YLIWLV | DNKPDVERK | | | | |
| | | | DMYHK | YGTVNTTLL | | | | |
| | | | HPNQR | DDLPGKAFS | | | | |
| | | | GTNCP | QYLHREGYD | | | | |
| | | | NVAWK | SIDEATLTLD | | | | |
| | | | RGCEE | EIKEIYLIWLV | | | | |
| | | | WEPEEF | DMYHKHPN | | | | |
| | | | TGTTAE | QRGTNCPN | | | | |
| | | | LDFKFA | VAWKRGCE | | | | |
| | | | VLDEKK | EWEPEEFTG | | | | |
| | | | LSKSGIT | TTAELDFKFA | | | | |
| | | | VYVDLT | VLDEKKLSKS | | | | |
| | | | YSSDRL | GITVYVDLTY | | | | |
| | | | AEYRGT | SSDRLAEYR | | | | |
| | | | HGNHM | GTHGNHMV | | | | |
| | | | VTFKYN | TFKYNPECM | | | | |
| | | | PECMG | GVIWVLDED | | | | |
| | | | VIWVLD | VDEYFTVPAI | | | | |
| | | | EDVDEY | DYDYASGVS | | | | |
| | | | FTVPAI | LWQHKYNIK | | | | |
| | | | DYDYAS | YQRSLNLSEY | | | | |
| | | | GVSLW | DEDFEVDAEI | | | | |
| | | | QHKYNI | RIEDIAEESIV | | | | |
| | | | KYQRSL | KTKKLRNRR | | | | |
| | | | NLSEYD | RGARYQENA | | | | |
| | | | EDFEVD | ERAKAQNQ | | | | |
| | | | AEIRIED | NAIIKTEQED | | | | |
| | | | IAEESIV | PQEEEVDDE | | | | |
| | | | KTKKLR | NAWGIDYL* | | | | |
| | | | NRRRG | (SEQ ID | | | | |
| | | | ARYQEN | NO: 143) | | | | |
| | | | AERAKA | | | | | |
| | | | QNQNA | | | | | |
| | | | IIKTEQE | | | | | |
| | | | DPQEEE | | | | | |
| | | | VDDEN | | | | | |
| | | | AWGID | | | | | |
| | | | YL (SEQ | | | | | |
| | | | ID | | | | | |
| | | | NO: 142) | | | | | |
| 23 | V. | MYVRTL | MNFPF | MYVRTLKQS | MHALSSAQK | MHALSSAQ | MLNPIELYED | MPKKKRKV |
| | fisc. | KQSQVK | DDEFQK | QVKNISKFM | EQLINFNQC | KEQLINFN | ESLESCLLRIS | GSGEQKLIS |
| | MJ11 | NISKFM | IINISGE | SLKNDSIIRTE | FIEYPIITHIYS | QCFIEYPIIT | QNNYYDSFQ | EEDLEQKLI |
| | GCA | SLKNDSI | QNKVIR | SMLEFDMCF | IFDDLRLNQ | HIYSIFDDLR | DFSDEVWFH | SEEDLEQKL |
| | 0000208 | IRTESM | NEEANS | HLEYSPDVVS | GLGAEPQC | LNQGLGAE | VKEEDREVR | ISEEDLGSG |
| | 451 | LEFDMC | IQLSLDS | FESQPQGFY | MLLLGDTGS | PQCMLLLG | GTFPATLNT | LNPIELYED |
| | | FHLEYS | YSHDIK | YKYQGKHLP | GKSALINNYL | DTGSGKSA | VNLYHSHTS | ESLESCLLRI |
| | | PDVVSF | MEVLRR | YTPDFLITHS | LQQPSSNFS | LINNYLLQQ | SDLKLKALIKI | SQNNYYDS |
| | | ESQPQ | ISFIKWI | SGLQQLLEIK | ALSSLPVLHT | PSSNFSALS | EQWLEINNF | FQDFSDEV |
| | | GFYYKY | KPRLKG | PLSKTQRPDF | RIPRRVNNE | SLPVLHTRI | PLLKSALSRS | WFHVKEED |
| | | QGKHLP | GLTEKN | QSKFIQKQQ | QTMYQLLTD | PRRVNNEQ | SNTFLRQHS | REVRGTFP |
| | | YTPDFLI | LKPLLSD | AAQKLNLSLI | LGQSPSGTR | TMYQLLTD | AVFRNGVDI | ATLNTVNLY |
| | | THSSGL | ASIHLK | LITEKQIRTG | RTKRSEIALA | LGQSPSGT | PRILLRKNGI | HSHTSSDLK |
| | | QQLLEI | MKAPC | HLLNNFKLLH | EGVVRALKR | RRTKRSEIA | PVCPECLKE | LKALIKIEQ |
| | | KPLSKT | TSTFIA | RYSGLHSISA | KKTELIIINEF | LAEGVVRA | NEYIRQEWH | WLEINNFPL |
| | | QRPDF | WCNRY | TQKAIINLIQ | QELIEFSSAR | LKRKKTELIII | FITHDVCTKH | LKSALSRSS |
| | | QSKFIQ | RLSGEK | KVNKIQISQI | ERQNVANTL | NEFQELIEF | KTDLLHHCP | NTFLRQHS |
| | | KQQAA | VSSLIPQ | ANSLNISNG | KYISEEARVSI | SSARERQN | ECKTSINYQE | AVFRNGVD |
| | | QKLNLS | HSQKG | EALTGVLSW | VLVGMPYA | VANTLKYIS | SENITDCQC | IPRILLRKNG |
| | | LILITEK | NRKLKT | LSKGALQTD | DIIAKEPQW | EEARVSIVL | GFKFSDHLTP | IPVCPECLK |
| | | QIRTGH | SSEFYIA | YSNEAITGNS | GSRLAWKTQ | VGMPYADII | QANSNALLI | ENEYIRQE |
| | | LLNNFK | KAINEK | YVWLGSGPK | IEYFSLKNDM | AKEPQWGS | AQWLNSEN | WHFITHDV |
| | | LLHRYS | YLTRNQ | KKRKVGSGY | KTYVQFLKGL | RLAWKTQI | TKLANVWG | CTKHKTDLL |
| | | GLHSIS | CSIIQAF | PYDVPDYAY | ANRMGYAE | EYFSLKND | EHQAISSRFG | HHCPECKT |
| | | ATQKAII | KYYCDLI | PYDVPDYAY | VPSLHSKELA | MKTYVQFL | VLLWYINRY | SINYQESEN |
| | | NLIQKV | IIENRST | PYDVPDYAG | IPLFSICRGEL | KGLANRM | NLTDDFSTSF | ITDCQCGFK |
| | | NKIQIS | PTNKIK | SGNFPFDDE | RQLKNFCSD | GYAEVPSL | VKYSLNWPT | FSDHLTPQ |
| | | QIANSL | KISQRTF | FQKIINISGE | AMLESFKQN | HSKELAIPLF | NFYSELDEQI | ANSNALLIA |
| | | NISNGE | YNRINA | QNKVIRNEE | KNTLTHYVLS | SICRGELRQ | DKAKTVQIK | QWLNSENT |
| | | ALTGVL | LPKYEV | ANSIQLSLDS | ATFKYKYPTK | LKNFCSDA | PFNKIFFNEIF | KLANVWGE |
| | | SWLSKG | ALKRYG | YSHDIKMEV | KNPFEMNVE | MLESFKQN | NRLLLDCRHL | HQAISSRFG |
| | | ALQTDY | KRYADI | LRRISFIKWIK | DIPIQEVISYS | KNTLTHYVL | PTREFKTNSI | VLLWYINRY |
| | | SNEAIT | NYRKVG | PRLKGGLTEK | KYNLDEMD | SATFKYKYP | LSHIYQYFLS | NLTDDFSTS |
| | | GNSYV | KIREATR | NLKPLLSDAS | DNKRLISTKY | TKKNPFEM | RYQIQPNSG | FVKYSLNW |
| | | WL | PLEYVEI | IHLKMKAPC | SDALPLTVILS | NVEDIPIQE | VFSILLSPLEA | PTNFYSELD |
| | | (SEQ ID | DHTPLD | TSTFIAWCN | QS (SEQ ID | VISYSGSGP | STLLSCTTDQ | EQIDKAKTV |
| | | NO: 148) | LILLDDE | RYRLSGEKVS | NO: 151) | AAKKKKLD | IYRLYELGFLK | QIKPFNKIFF |
| | | | LEIPLGR | SLIPQHSQK | | GSGKYNLD | LGVRPKLHQ | NEIFNRLLL |
| | | | PYLTILI | GNRKLKTSSE | | EMDDNKRL | KIASHQSVFT | DCRHLPTR |
| | | | DRYSKCI | FYIAKAINEK | | ISTKYSDAL | LSSIILVKLSN | EFKTNSILS |
| | | | IGYNISF | YLTRNQCSII | | PLTVILSQS* | MQSSQDEL | HIYQYFLSR |
| | | | RPPSFE | QAFKYYCDLI | | (SEQ ID | HHYLSAW | YQIQPNSG |
| | | | SIRHAF | IIENRSTPTN | | NO: 152) | (SEQ ID | VFSILLSPLE |
| | | | CNACLD | KIKKISQRTFY | | | NO: 153) | ASTLLSCTT |
| | | | KSSITQ | NRINALPKYE | | | | DQIYRLYEL |
| | | | QYPHLK | VALKRYGKR | | | | GFLKLGVRP |
| | | | NDWP | YADINYRKV | | | | KLHQKIASH |
| | | | MAGKIE | GKIREATRPL | | | | QSVFTLSSII |
| | | | NLVVD | EYVEIDHTPL | | | | LVKLSNMQ |
| | | | NGAEF | DLILLDDELEI | | | | SSQDELHH |
| | | | WSNSLE | PLGRPYLTILI | | | | YLSAW |
| | | | DSLRPF | DRYSKCIIGY | | | | (SEQ ID |
| | | | ATNILF | NISFRPPSFE | | | | NO: 154) |
| | | | NKVGKP | SIRHAFCNAC | | | | |
| | | | WMKPL | LDKSSITQQY | | | | |
| | | | VEKFFD | PHLKNDWP | | | | |
| | | | VLNKEL | MAGKIENLV | | | | |
| | | | VHSLPG | VDNGAEFW | | | | |
| | | | TTRSRV | SNSLEDSLRP | | | | |
| | | | EQLKGY | FATNILFNKV | | | | |
| | | | NPKKDA | GKPWMKPL | | | | |
| | | | AITFSLF | VEKFFDVLN | | | | |
| | | | LELFHT | KELVHSLPGT | | | | |
| | | | WIIDIYH | TRSRVEQLK | | | | |
| | | | MTPDT | GYNPKKDAA | | | | |
| | | | RGVSIP | ITFSLFLELFH | | | | |
| | | | YFKWQ | TWIIDIYHMT | | | | |
| | | | EGIKNL | PDTRGVSIPY | | | | |
| | | | PPLSFS | FKWQEGIKN | | | | |
| | | | NEEAQ | LPPLSFSNEE | | | | |
| | | | QLLIEFG | AQQLLIEFGI | | | | |
| | | | ILNTRTL | LNTRTLTIHG | | | | |
| | | | TIHGISI | ISIHNKRYQS | | | | |
| | | | HNKRY | DELIEYRKKY | | | | |
| | | | QSDELIE | GNIKENNLRL | | | | |
| | | | YRKKYG | KTKTNPSNIS | | | | |
| | | | NIKENN | YIFVYLPNEA | | | | |
| | | | LRLKTKT | RYIKVPCTDG | | | | |
| | | | NPSNIS | DSYIKNLTLY | | | | |
| | | | YIFVYLP | QHNVISKLTR | | | | |
| | | | NEARYI | TKTSLQENKE | | | | |
| | | | KVPCTD | DQADSRMYI | | | | |
| | | | GDSYIK | DKRIGKQLEK | | | | |
| | | | NLTLYQ | IQENKKNIGK | | | | |
| | | | HNVISK | IKHISKIACYQ | | | | |
| | | | LTRTKTS | NIGSHTQKSL | | | | |
| | | | LQENKE | QFPTLNDNT | | | | |
| | | | DQADS | KSEYKDRILN | | | | |
| | | | RMYIDK | NWNEQFDD | | | | |
| | | | RIGKQL | LEGF* (SEQ | | | | |
| | | | EKIQEN | ID NO: 150) | | | | |
| | | | KKNIGKI | | | | | |
| | | | KHISKIA | | | | | |
| | | | CYQNIG | | | | | |
| | | | SHTQKS | | | | | |
| | | | LQFPTL | | | | | |
| | | | NDNTKS | | | | | |
| | | | EYKDRIL | | | | | |
| | | | NNWNE | | | | | |
| | | | QFDDLE | | | | | |
| | | | GF (SEQ | | | | | |
| | | | ID | | | | | |
| | | | NO: 149) | | | | | |
| 24 | V. | MSALPS | MPKKSF | MSALPSPST | MLRNHQM | MLRNHQM | MLLQRPKPH | MPKKKRKV |
| | paraISF- | PSTATLI | SSFHRK | ATLIALESAF | NETREARISK | NETREARIS | SDESLESYLIR | GSGEQKLIS |
| | 25-6 | ALESAF | SALQQE | DTPARNLTK | AKRAFVSTPS | KAKRAFVST | VANKNGYES | EEDLEQKLI |
| | | DTPARN | KPEPDE | SRGKNIHRY | VTKILCYMD | PSVTKILCY | TGRFLISLKSY | SEEDLEQKL |
| | | LTKSRG | RVVDTS | VSAKMGKR | RCRDLSDFD | MDRCRDLS | LCDIDSHRFA | ISEEDLGSG |
| | | KNIHRY | DVDEET | VTVESFLECA | SEPTCMMV | DFDSEPTC | SFPTDIRLIHP | LLQRPKPHS |
| | | VSAKM | YRDISAF | ACYHFDFEP | YGASGVGKT | MMVYGAS | YSSQRSSSTR | DESLESYLIR |
| | | GKRVTV | PDNIAT | SIVRFCSQPI | TIIKKYLNQN | GVGKTTIIK | SHALQHISQL | VANKNGYE |
| | | ESFLEC | QITFRLS | RLSYCLNGK | RRDSDVGG | KYLNQNRR | TFTEAPELLG | STGRFLISLK |
| | | AACYHF | ILRYLAS | AHTYVPDFL | DVIPVLHIEL | DSDVGGDV | LAISRSPLKYS | SYLCDIDSH |
| | | DFEPSIV | KCEKIIP | VQFDTGEYT | PDNAKPVDA | IPVLHIELPD | PSTTSLIRAD | RFASFPTDI |
| | | RFCSQP | KTIEPH | LYEVKSDME | ARELLVEMG | NAKPVDAA | EIFPKSLIRTK | RLIHPYSSQ |
| | | IRLSYCL | RVALQR | SSKSEFQCE | DPLAIYETDL | RELLVEMG | HVPCCTSCL | RSSSTRSHA |
| | | NGKAH | LHDRNI | WEAKVQGA | ARLTKKLVDL | DPLAIYETD | NEQGYANYL | LQHISQLTF |
| | | TYVPDF | PSAISIY | FELGLELELV | IPVVGVKLIII | LARLTKKLV | WHFEGYNC | TEAPELLGL |
| | | LVQFDT | RWWLV | TEEEILDEVIF | DEFQHLVEE | DLIPVVGVK | CHIHEKPLTY | AISRSPLKYS |
| | | GEYTLY | FRASDC | SNLRLLHRYA | RSNRVLTQV | LIIIDEFQHL | QCECGEPYD | PSTTSLIRA |
| | | EVKSD | NPVSLA | SRDNLNHFH | GNWLKRILN | VEERSNRVL | YRIYGLKLVC | DEIFPKSLIR |
| | | MESSKS | PRNKDK | QTLLTTLKLN | KTKCPIVLFG | TQVGNWL | PSCGSILTHQ | TKHVPCCTS |
| | | EFQCE | GNSKVK | GTQTAKSLG | MPYSKVVLQ | KRILNKTKC | GGEPESTSVE | CLNEQGYA |
| | | WEAKV | LPKFVD | HHLGLNERKI | ANSQLHGRF | PIVLFGMPY | IAQWLAGLT | NYLWHFEG |
| | | QGAFEL | ALMKQ | FPFLCDLLSR | SIQFELRPFSY | SKVVLQAN | TEPFPEIPAS | YNCCHIHEK |
| | | GLELEL | AVERVI | NLLQTSLETP | QGGKGVFN | SQLHGRFSI | YRWGLIHW | PLTYQCEC |
| | | VTEEEIL | SGRKVR | LSLESEFELG | TFLEYLDKAL | QFELRPFSY | WMKIQNTE | GEPYDYRIY |
| | | DEVIFS | IRSAYKR | CYAGSGPKK | PFERQAGLA | QGGKGVFN | ALDTGSFSTF | GLKLVCPSC |
| | | NLRLLH | VRRKLR | KRKVGSGYP | NESLQKKLYA | TFLEYLDKA | WQQWPESF | GSILTHQG |
| | | RYASRD | QHNLN | YDVPDYAYP | FSQGNMRSL | LPFERQAGL | HNLIEQTLN | GEPESTSVE |
| | | NLNHFH | NGTKYK | YDVPDYAYP | RNLIYQASIE | ANESLQKKL | HNQEYSVLA | IAQWLAGL |
| | | QTLLTTL | YPTYESL | YDVPDYAGS | AIDNQHATIT | YAFSQGN | PHQWRLKD | TTEPFPEIP |
| | | KLINGTQ | RKRVKK | GPKKSFSSFH | EEDFVFASKL | MRSLRNLIY | LVGELLFSSI | ASYRWGLI |
| | | TAKSLG | KTPFELL | RKSALQQEK | TSGDKPITW | QASIEAIDN | NLPSRNLKY | HWWMKIQ |
| | | HHLGLN | AAKKGE | PEPDERVVD | KNPFDEGVK | QHATITEED | NLPLRELFCY | NTEALDTG |
| | | ERKIFPF | RVAKRE | TSDVDEETY | VTEDMLRPP | FVFASKLTS | LENHLWEYN | SFSTFWQQ |
| | | LCDLLSR | FRRMG | RDISAFPDNI | PKDIGWEDY | GDKPITWK | GLIANLKLNA | WPESFHNLI |
| | | NLLQTS | KKILTSY | ATQITFRLSIL | YHNVKPKNQ | NPFDEGVK | FDAATVLNC | EQTLNHNQ |
| | | LETPLSL | VLERVEI | RYLASKCEKII | RRKGGNIFE | VTEDMLRP | DTEQIASMA | EYSVLAPH |
| | | ESEFEL | DHTVV | PKTIEPHRVA | (SEQ ID | PPKDIGSGP | EQGVLVPLW | QWRLKDLV |
| | | GCYA | DLFAVH | LQRLHDRNI | NO: 158) | AAKKKKLD | SRKREELISYT | GELLFSSINL |
| | | (SEQ ID | EEHRVP | PSAISIYRW | | GSGGWED | DYLFHFGDV | PSRNLKYNL |
| | | NO: 155) | LGRPW | WLVFRASDC | | YYHNVKPK | FCLWLAEFQ | PLRELFCYL |
| | | | LTQLVD | NPVSLAPRN | | NQRRKGG | TDEFNRSFYT | ENHLWEYN |
| | | | CYSKAVI | KDKGNSKVK | | NIFE* (SEQ | SRW (SEQ ID | GLIANLKLN |
| | | | GFYLGF | LPKFVDALM | | ID NO: 159) | NO: 160) | AFDAATVL |
| | | | EPPSYV | KQAVERVIS | | | | NCDTEQIAS |
| | | | SVSLAL | GRKVRIRSAY | | | | MAEQGVL |
| | | | KNAILR | KRVRRKLRQ | | | | VPLWSRKR |
| | | | KDDLLS | HNLNNGTKY | | | | EELISYTDYL |
| | | | SFDSVE | KYPTYESLRK | | | | FHFGDVFCL |
| | | | NEWLC | RVKKKTPFEL | | | | WLAEFQTD |
| | | | YGIPDLL | LAAKKGERV | | | | EFNRSFYTS |
| | | | VTDNG | AKREFRRMG | | | | RW (SEQ ID |
| | | | KEFLSK | KKILTSYVLER | | | | NO: 161) |
| | | | AFDKAC | VEIDHTVVDL | | | | |
| | | | ESLLINV | FAVHEEHRV | | | | |
| | | | HQNRV | PLGRPWLTQ | | | | |
| | | | ETPDNK | LVDCYSKAVI | | | | |
| | | | PHVERN | GFYLGFEPPS | | | | |
| | | | YGTINT | YVSVSLALKN | | | | |
| | | | SLLDDL | AILRKDDLLS | | | | |
| | | | PGKAFS | SFDSVENEW | | | | |
| | | | QYLHRE | LCYGIPDLLV | | | | |
| | | | GYDSVG | TDNGKEFLS | | | | |
| | | | EATLTL | KAFDKACESL | | | | |
| | | | DEIKEIY | LINVHQNRV | | | | |
| | | | LIWLVDI | ETPDNKPHV | | | | |
| | | | YHKNSN | ERNYGTINTS | | | | |
| | | | QRGTN | LLDDLPGKAF | | | | |
| | | | CPNVA | SQYLHREGY | | | | |
| | | | WKRGS | DSVGEATLTL | | | | |
| | | | QEWEP | DEIKEIYLIWL | | | | |
| | | | EEFTGS | VDIYHKNSN | | | | |
| | | | KDELDF | QRGTNCPN | | | | |
| | | | KFAIVE | VAWKRGSQ | | | | |
| | | | HKQLTK | EWEPEEFTG | | | | |
| | | | AGVTVY | SKDELDFKFA | | | | |
| | | | KELTYSS | IVEHKQLTKA | | | | |
| | | | ERLAEY | GVTVYKELTY | | | | |
| | | | RGKKG | SSERLAEYRG | | | | |
| | | | NHKVQ | KKGNHKVQF | | | | |
| | | | FKYNPE | KYNPECMAV | | | | |
| | | | CMAVI | IWVLDEDQN | | | | |
| | | | WVLDE | EYFTVNAIDY | | | | |
| | | | DQNEYF | EYASRVSLW | | | | |
| | | | TVNAID | QHKYNMKY | | | | |
| | | | YEYASR | QAELNSAEY | | | | |
| | | | VSLWQ | DEDKEIDADI | | | | |
| | | | HKYNM | KIEEIADRSIV | | | | |
| | | | KYQAEL | KTNKIRARRR | | | | |
| | | | NSAEYD | GARHQENS | | | | |
| | | | EDKEID | ARAKSISDAK | | | | |
| | | | ADIKIEE | PVPPQKHEE | | | | |
| | | | IADRSIV | ETVIFDNED | | | | |
| | | | KTNKIR | WDIDYV* | | | | |
| | | | ARRRGA | (SEQ ID | | | | |
| | | | RHQEN | NO: 157) | | | | |
| | | | SARAKSI | | | | | |
| | | | SDAKPV | | | | | |
| | | | PPQKHE | | | | | |
| | | | EETVIFD | | | | | |
| | | | NEDWD | | | | | |
| | | | IDYV | | | | | |
| | | | (SEQ ID | | | | | |
| | | | NO: 156) | | | | | |
| 25 | V. | MYVRN | MVMPF | MYVRNLRKP | MNLSAKQEI | MNLSAKQE | VETDIQLYPD | MPKKKRKV |
| | cholerae | LRKPSA | DDEFESI | SATKNVYKF | AVDELLTQY | IAVDELLTQ | ESLESFLLRLS | GSGEQKLIS |
| | YB2A06_ | TKNVYK | NDDTQ | ASSKNRSVIL | HNSFVIYPDV | YHNSFVIYP | QEQGYERFS | EEDLEQKLI |
| | GCA_ | FASSKN | AEYDST | CESSLERDCC | QQIFDGLD | DVQQIFDG | HFAEDIWFD | SEEDLEQKL |
| | 001402 | RSVILCE | SEAKLV | YHLEYSKDVF | WIVRRSQFG | LDWIVRRS | TLDQHEAIP | ISEEDLGSG |
| | 375.1 | SSLERD | RKQYLP | SFQSQPEGF | NFTPSMLITG | QFGNFTPS | GAFPLELNRI | ETDIQLYPD |
| | | CCYHLE | LDSVTI | YYSSGNKRC | GTGAGKTSLI | MLITGGTG | NIYHAQTTS | ESLESFLLRL |
| | | YSKDVF | HERDLS | PYTPDFLVR | NHYAKYHFN | AGKTSLINH | QMRVRVLIH | SQEQGYER |
| | | SFQSQP | SFSEEQ | NQDGSEYYL | DNEVLITRVR | YAKYHEND | LENQLKLNN | FSHFAEDI |
| | | EGFYYS | KNKALE | EVKPLAKTFS | PSFIETLIWAI | NEVLITRVR | FGALRLALSH | WFDTLDQ |
| | | SGNKRC | RYKLISA | EDFKRSFALK | DKLGIPYNTR | PSFIETLIW | SKAQFSPEYK | HEAIPGAFP |
| | | PYTPDF | VAKEIS | RIAAQHQGK | SKRSEIGLQD | AIDKLGIPY | AVHRFEADY | LELNRINIY |
| | | LVRNQ | GGWTP | LLILVTDKQIR | YFINSVKKSN | NTRSKRSEI | PFVFLAKRFT | HAQTTSQ |
| | | DGSEYY | KNINPLI | NGVYLENLN | LKLLVIEEAQ | GLQDYFINS | PICPLCISEAP | MRVRVLIH |
| | | LEVKPL | DKYGLN | LIHRYSGLVD | ELFECASPKE | VKKSNLKLL | YIRQQWQFL | LENQLKLN |
| | | AKTFSE | LSIKRPS | FSLSSTKIVEE | RQKIRDRLK | VIEEAQELF | SQQACERHG | NFGALRLAL |
| | | DFKRSF | YKSVIR | LSAAGRMCI | MISDECRLPI | ECASPKER | CKLVHHCPE | SHSKAQFSP |
| | | ALKRIA | WYKSFC | RSLADNLKLS | VFIGIPTAKLI | QKIRDRLK | CQSRLEYQT | EYKAVHRFE |
| | | AQHQG | GSDGNI | IGEVIAVVFR | LEDSQWDR | MISDECRLP | TESISQCECG | ADYPFVFLA |
| | | KLLILVT | VCLVDH | LIGLGRVNVP | RIMVKRELPY | IVFIGIPTAK | FELRNSPVED | KRFTPICPL |
| | | DKQIRN | NHSKG | LDSAINEMS | IRITSESSLDV | LILEDSQW | APVAALLVA | CISEAPYIR |
| | | GVYLEN | NRTKRII | VISVNGSGP | YIDLLEELEK | DRRIMVKR | RWLSGNDSK | QQWQFLS |
| | | LNLIHRY | DDESFF | KKKRKVGSG | QLPISVQPEL | ELPYIRITSE | PLGLLKAEM | QQACERHG |
| | | SGLVDF | VEATER | YPYDVPDYA | SEMDIAMRL | SSLDVYIDLL | TLSERYGFLL | CKLVHHCP |
| | | SLSSTKI | FLDAKR | YPYDVPDYA | LSATKGMLG | EELEKQLPIS | WYVNRYGDI | ECQSRLEYQ |
| | | VEELSA | PNYSQA | YPYDVPDYA | AIKELVGYAL | VQPELSEM | ENISFESFVE | TTESISQCE |
| | | AGRMCI | YQFYCD | GSGVMPFD | ELALLSGKSA | DIAMRLLSA | YCSCWPRVL | CGFELRNSP |
| | | RSLADN | RIEIENS | DEFESINDDT | ITNDEFALGF | TKGMLGAI | QEELDELVN | VEDAPVAA |
| | | LKLSIGE | NIISGQI | QAEYDSTSE | ERINGPDVT | KELVGYALE | KADLIRVKD | LLVARWLS |
| | | VIAVVF | SKVSYQ | AKLVRKQYL | NPFTTELEKL | LALLSGKSAI | WKKTFFNEV | GNDSKPLG |
| | | RLIGLG | AFKERL | PLDSVTIHER | LVPQVIEYEG | TNDEFALG | FGALLKDCR | LLKAEMTLS |
| | | RVNVPL | KKLPPY | DLSSFSEEQK | FIIDPENGEIK | FERINGPDV | QLPSRQLNR | ERYGFLLW |
| | | DSAINE | EVALKR | NKALERYKLI | FTKQIFKDIPL | TNPFTTELE | NSVLTQVLA | YVNRYGDIE |
| | | MSVISV | FGPNYA | SAVAKEISGG | AALLG (SEQ | KLLVPQVIE | YFTKLMATL | NISFESFVEY |
| | | N (SEQ | NKLFNY | WTPKNINPLI | ID NO: 165) | YEGSGPAA | PSSSKGNVG | CSCWPRVL |
| | | ID | YQSSVP | DKYGLNLSIK | | KKKKLDGS | DVLLSPLEVS | QEELDELV |
| | | NO: 162) | TTRILER | RPSYKSVIR | | GGFIIDPEN | TLLSCTTDEV | NKADLIRVK |
| | | | VELDHT | WYKSFCGSD | | GEIKFTKQIF | YRLYEFGEIK | DWKKTFFN |
| | | | PLDLILL | GNIVCLVDH | | KDIPLAALL | AAIRPRMHT | EVFGALLKD |
| | | | DDDLLI | NHSKGNRTK | | G* (SEQ ID | KIASHESAFT | CRQLPSRQ |
| | | | PLGRAY | RIIDDESFFVE | | NO: 166) | LRSVIETKLTR | LNRNSVLT |
| | | | LTLLVD | ATERFLDAK | | | MCSENDGLS | QVLAYFTKL |
| | | | VFSGCI | RPNYSQAYQ | | | VYLPEW | MATLPSSSK |
| | | | VGFHLG | FYCDRIEIEN | | | (SEQ ID | GNVGDVLL |
| | | | FNPPSY | SNIISGQISKV | | | NO: 167) | SPLEVSTLLS |
| | | | VSVAKA | SYQAFKERLK | | | | CTTDEVYRL |
| | | | IIHSVKS | KLPPYEVALK | | | | YEFGEIKAAI |
| | | | KDYVH | RFGPNYANK | | | | RPRMHTKI |
| | | | DLNIELT | LFNYYQSSVP | | | | ASHESAFTL |
| | | | NDWLC | TTRILERVEL | | | | RSVIETKLTR |
| | | | HGKME | DHTPLDLILL | | | | MCSENDGL |
| | | | TLVVDN | DDDLLIPLGR | | | | SVYLPEW |
| | | | GAEFW | AYLTLLVDVF | | | | |
| | | | SKSLDQ | SGCIVGFHLG | | | | |
| | | | ACMEA | FNPPSYVSV | | | | |
| | | | GIHYEY | AKAIIHSVKS | | | | |
| | | | CKVGQ | KDYVHDLNI | | | | |
| | | | PWEKP | ELTNDWLCH | | | | |
| | | | RVERKF | GKMETLVVD | | | | |
| | | | LEIIQGI | NGAEFWSKS | | | | |
| | | | VGWVP | LDQACMEA | | | | |
| | | | GKTFSN | GIHYEYCKVG | | | | |
| | | | ILEKDRY | QPWEKPRVE | | | | |
| | | | DPQKD | RKFLEIIQGIV | | | | |
| | | | AVMRF | GWVPGKTFS | | | | |
| | | | SSFVEEL | NILEKDRYDP | | | | |
| | | | HRWIID | QKDAVMRF | | | | |
| | | | VHNASP | SSFVEELHR | | | | |
| | | | DSRNTK | WIIDVHNAS | | | | |
| | | | IPNYHW | PDSRNTKIPN | | | | |
| | | | KKSEEA | YHWKKSEEA | | | | |
| | | | LPPAAL | LPPAALSDR | | | | |
| | | | SDRDEK | DEKQFRIIM | | | | |
| | | | QFRIIM | GVIHEGVVT | | | | |
| | | | GVIHEG | TKGIKYKHL | | | | |
| | | | VVTTKG | MYDNVALE | | | | |
| | | | IKYKHL | QYRKQYPQT | | | | |
| | | | MYDNV | KESRKKTIKID | | | | |
| | | | ALEQYR | PDDLSSIFVY | | | | |
| | | | KQYPQT | LEEIGGYIEV | | | | |
| | | | KESRKK | PCKYDPLGYT | | | | |
| | | | TIKIDPD | KNLSLSEHVR | | | | |
| | | | DLSSIFV | ITKIHRDFIKG | | | | |
| | | | YLEEIGG | QVDALSLAK | | | | |
| | | | YIEVPCK | ARQALHERIK | | | | |
| | | | YDPLGY | TEQEHLSLM | | | | |
| | | | TKNLSLS | SVESRAKKA | | | | |
| | | | EHVRIT | KHGKKMAA | | | | |
| | | | KIHRDFI | LSGISNEQP | | | | |
| | | | KGQVD | MSIQNALEN | | | | |
| | | | ALSLAK | KNKPLDDNF | | | | |
| | | | ARQALH | DEPTPVDNL | | | | |
| | | | ERIKTE | KSLWNKRKA | | | | |
| | | | QEHLSL | MKRSKE* | | | | |
| | | | MSVESR | (SEQ ID | | | | |
| | | | AKKAKH | NO: 164) | | | | |
| | | | GKKMA | | | | | |
| | | | ALSGIS | | | | | |
| | | | NEQPM | | | | | |
| | | | SIQNAL | | | | | |
| | | | ENKNKP | | | | | |
| | | | LDDNFD | | | | | |
| | | | EPTPVD | | | | | |
| | | | NLKSLW | | | | | |
| | | | NKRKA | | | | | |
| | | | MKRSKE | | | | | |
| | | | (SEQ ID | | | | | |
| | | | NO: 163) | | | | | |
| 26 | Agari- | MKSRVI | MASSR | MKSRVIGPS | MAQLLEMQ | MAQLLEM | MFLIPEDYHE | MPKKKRKV |
| | vorans | GPSTHK | HTLGLF | THKSIFKFAS | QSQFDSFLD | QQSQFDSF | DESLESYLLRI | GSGEQKLIS |
| | gilvus | SIFKFAS | DDEYDS | PKMGKMVK | CFIEHPTVTTI | LDCFIEHPT | SQANGFESY | EEDLEQKLI |
| | strain | PKMGK | LSAESIE | VESSLEYDAC | YEIFDRLRFH | VTTIYEIFDR | ALLSGAVKEF | SEEDLEQKL |
| | WH0801 | MVKVE | SFSKETS | FHFEYSPSITS | FHSHQRISA | LRFHFHSH | LRQHDAEAY | ISEEDLGSG |
| | | SSLEYD | DLDND | FIAQPCGVD | GAAADVPC | QRISAGAA | GAFPLELSLV | FLIPEDYHE |
| | | ACFHFE | HLSPDF | YQLNGRTQT | MLLTGDSGS | ADVPCMLL | NIYHAKLSSS | DESLESYLL |
| | | YSPSITS | DSYSKE | FYPDFLVEDK | GKSSLVRHY | TGDSGSGK | FRVRAIRLM | RISQANGFE |
| | | FIAQPC | QQREAL | EFGKRFFEIK | RQQAQASP | SSLVRHYR | EELIGLSTWQ | SYALLSGAV |
| | | GVDYQL | RRYALI | PSSKVRKPEF | DSQLNVTPV | QQAQASPD | LNRLALKHT | KEFLRQHD |
| | | NGRTQ | QWVDK | RVKFALRRE | LVTRIPDTPS | SQLNVTPVL | AQTIVGSYTI | AEAYGAFPL |
| | | TFYPDF | RLKGG | AALSQSIPLIV | LDLTILEMLS | VTRIPDTPS | LVRQKEFLPR | ELSLVNIYH |
| | | LVEDKE | WTEKKL | VTEKQICLNP | TLGHFGTSFR | LDLTILEML | AFLRQGSVP | AKLSSSFRV |
| | | FGKRFF | SPLLEQ | ILNNLKLLHR | YKASNSLSLT | STLGHFGTS | VCPQCLSVQ | RAIRLMEEL |
| | | EIKPSSK | ATIEFDF | YAGNYSLTPL | ASLLKALAYK | FRYKASNSL | PYIRQNWHF | IGLSTWQL |
| | | VRKPEF | TLPNW | HFWVLDAV | KTELIIINEVQ | SLTASLLKAL | LPCTACNLH | NRLALKHT |
| | | RVKFAL | RTLSRW | KSLGRITVRD | ELFEFKSLKE | AYKKTELIII | QTKLLCHCPE | AQTIVGSYT |
| | | RREAAL | YSSYINS | LVDESDCAP | CTAISNRLKYI | NEVQELFEF | CGEALNYQK | ILVRQKEFL |
| | | SQSIPLI | GHSLEA | GDVFASALT | SEESGIPFVL | KSLKECTAIS | TELIEYCQCG | PRAFLRQG |
| | | VVTEKQ | LLPKHH | WISRGHLQA | VGMPWADK | NRLKYISEES | YDLRSVRTN | SVPVCPQC |
| | | ICLNPIL | KKGGTG | DISDNELGV | ITDDPQWDS | GIPFVLVG | VASKAECQL | LSVQPYIRQ |
| | | NNLKLL | ARKME | NSLVWCGS | RLIHKQFLPY | MPWADKIT | SAIFDKSREA | NWHFLPCT |
| | | HRYAG | DGFFFE | GPKKKRKVG | FNLSSKSDLK | DDPQWDS | SNNPLLVCR | ACNLHQTK |
| | | NYSLTP | KAIEEYY | SGYPYDVPD | EFSRLINGFC | RLIHKQFLP | HTSIRTGALL | LLCHCPECG |
| | | LHFWVL | LTRERP | YAYPYDVPD | LRMGFDVPP | YFNLSSKSD | WYCLWRNV | EALNYQKT |
| | | DAVKSL | TIADCY | YAYPYDVPD | KLNDKHTIRA | LKEFSRLIN | ELDELVVDK | ELIEYCQCG |
| | | GRITVR | ELYKSW | YAGSGASSR | LFSACSGQM | GFCLRMGF | NHAQDCIGF | YDLRSVRTN |
| | | DLVDES | IVLENSK | HTLGLFDDE | RSLKSLLSEAL | DVPPKLND | FERWPDEIN | VASKAECQ |
| | | DCAPG | LISGKLK | YDSLSAESIES | FLALKDRALT | KHTIRALFS | KELAAIAEAA | LSAIFDKSR |
| | | DVFASA | PVCQRT | FSKETSDLDN | IELKHLEEAFI | ACSGQMRS | EQRLVEPFN | EASNNPLLV |
| | | LTWISR | FYNRIN | DHLSPDFDS | FQKPGVSNP | LKSLLSEALF | KTAFSAVFG | CRHTSIRTG |
| | | GHLQA | KLSPYLV | YSKEQQREA | FKMAFEEIPV | ALKDRALT | GLLNRSRVA | ALLWYCLW |
| | | DISDNE | ALRRFG | LRRYALIQW | PKVKEYSKLN | IELKHLEEAF | PLSMSSEDFI | RNVELDELV |
| | | LGVNSL | KPYADR | VDKRLKGG | HAASTLDEQI | IFQKPGVSN | HQSVIQFLV | VDKNHAQ |
| | | VWC | HFRTVK | WTEKKLSPLL | IRTQFVDGLP | PFKMAFEEI | HLVMDNPK | DCIGFFER |
| | | (SEQ ID | QLKKPS | EQATIEFDFT | ISQLLKKNS* | PVPKVKEYS | SKQPNIADL | WPDEINKE |
| | | NO: 169) | NVLERV | LPNWRTLSR | (SEQ ID | GSGPAAKK | QLTVPEVAA | LAAIAEAAE |
| | | | EIDHTPL | WYSSYINSG | NO: 172) | KKLDGSGKL | LLNCSREQV | QRLVEPFN |
| | | | DLILVD | HSLEALLPKH | | NHAASTLD | YRYYEEGML | KTAFSAVFG |
| | | | DELLLPL | HKKGGTGAR | | EQIIRTQFV | ELTFRLRLHN | GLLNRSRV |
| | | | GRPYLT | KMEDGFFFE | | DGLPISQLL | TLSLNKPAFF | APLSMSSE |
| | | | ALMDSY | KAIEEYYLTR | | KKNS* | LRQAVELAIS | DFIHQSVIQ |
| | | | SGCIVG | ERPTIADCYE | | (SEQ ID | LTSGSGDPLP | FLVHLVMD |
| | | | FYIGYRE | LYKSWIVLEN | | NO: 173) | AW (SEQ ID | NPKSKQPNI |
| | | | PSYDSV | SKLISGKLKP | | | NO: 174) | ADLQLTVPE |
| | | | RRALSC | VCQRTFYNRI | | | | VAALLNCSR |
| | | | AYLPKH | NKLSPYLVAL | | | | EQVYRYYEE |
| | | | WVKER | RRFGKPYAD | | | | GMLELTFRL |
| | | | FPSIKKE | RHFRTVKQL | | | | RLHNTLSLN |
| | | | WPCEG | KKPSNVLER | | | | KPAFFLRQA |
| | | | KIGMLV | VEIDHTPLDL | | | | VELAISLTSG |
| | | | VDNAA | ILVDDELLLPL | | | | SGDPLPAW |
| | | | EFWSSS | GRPYLTALM | | | | (SEQ ID |
| | | | LDDACA | DSYSGCIVGF | | | | NO: 175) |
| | | | GIVQNV | YIGYREPSYD | | | | |
| | | | DYNQV | SVRRALSCAY | | | | |
| | | | ARPWL | LPKHWVKER | | | | |
| | | | KPMIER | FPSIKKEWPC | | | | |
| | | | FFSTVN | EGKIGMLVV | | | | |
| | | | KKLLISIP | DNAAEFWSS | | | | |
| | | | GKTFSSI | SLDDACAGI | | | | |
| | | | QELKDY | VQNVDYNQ | | | | |
| | | | KPEKDA | VARPWLKP | | | | |
| | | | VMRFST | MIERFFSTVN | | | | |
| | | | FMELFH | KKLLISIPGKT | | | | |
| | | | KWLIDE | FSSIQELKDY | | | | |
| | | | YHYRPD | KPEKDAVMR | | | | |
| | | | TRETKIP | FSTFMELFH | | | | |
| | | | IVQWC | KWLIDEYHY | | | | |
| | | | KGTSLV | RPDTRETKIP | | | | |
| | | | SPPTYE | IVQWCKGTS | | | | |
| | | | ANEAER | LVSPPTYEAN | | | | |
| | | | LLIELAK | EAERLLIELAK | | | | |
| | | | VNERSV | VNERSVLHD | | | | |
| | | | LHDGIHI | GIHIHKLRYV | | | | |
| | | | HKLRYV | SDELTEYRKR | | | | |
| | | | SDELTE | KSPETGAKH | | | | |
| | | | YRKRKS | LKVKVKTIHT | | | | |
| | | | PETGAK | SIAYIFVFLQS | | | | |
| | | | HLKVKV | EQRYIKVPCV | | | | |
| | | | KTIHTSI | DQEYASGLS | | | | |
| | | | AYIFVFL | LLQHQTNQR | | | | |
| | | | QSEQRY | FVRSYVRSSV | | | | |
| | | | IKVPCV | DTEHLAECK | | | | |
| | | | DQEYAS | VYLHERIRKE | | | | |
| | | | GLSLLQ | AEALSQKVK | | | | |
| | | | HQTNQ | RKNPKIGGM | | | | |
| | | | RFVRSY | KKMAKYHNI | | | | |
| | | | VRSSVD | GSDSGNGSI | | | | |
| | | | TEHLAE | TAAQAIQTQ | | | | |
| | | | CKVYLH | TLLANNTKPT | | | | |
| | | | ERIRKEA | DIEDLDWEN | | | | |
| | | | EALSQK | FELEDGAY* | | | | |
| | | | VKRKNP | (SEQ ID | | | | |
| | | | KIGGMK | NO: 171) | | | | |
| | | | KMAKY | | | | | |
| | | | HNIGSD | | | | | |
| | | | SGNGSI | | | | | |
| | | | TAAQAI | | | | | |
| | | | QTQTLL | | | | | |
| | | | ANNTKP | | | | | |
| | | | TDIEDL | | | | | |
| | | | DWENF | | | | | |
| | | | ELEDGA | | | | | |
| | | | Y* (SEQ | | | | | |
| | | | ID | | | | | |
| | | | NO: 170) | | | | | |
| 27 | V. | MFDQT | MPPDS | MFDQTKKSS | LNLTPKQLE | MTMILKILK | VKTDIQHYS | MPKKKRKV |
| | cholerae | KKSSHV | NSIFGFF | HVHNICKFM | QLKSFETCFI | GISLNLTPK | DESLESFLLRL | GSGEQKLIS |
| | VC35_ | HNICKF | DEFEAS | SLKNDAVVR | EYPAITEIYSIF | QLEQLKSFE | SQEQGYERF | EEDLEQKLI |
| | GCA_ | MSLKN | EEESQL | TLSILEFDFCF | DQLRFNHSL | TCFIEYPAIT | SHFAEDIWF | SEEDLEQKL |
| | 0002994 | DAVVRT | LPKELIL | HLEYNPNIKS | GGEPESFLLT | EIYSIFDQLR | DTMEQHEAI | ISEEDLGSG |
| | 95.2 | LSILEFD | EPVEISS | FTSQPFGFH | GEAGSGKTA | FNHSLGGE | AGAFPLELN | KTDIQHYSD |
| | | FCFHLE | TIDSLPA | YLFNNRKCR | LINNYLSRFQ | PESFLLTGE | RINIYHAQTT | ESLESFLLRL |
| | | YNPNIK | KIQEEVL | YTPDFLAIGH | SGSTWGKQ | AGSGKTALI | SQMRVRVLI | SQEQGYER |
| | | SFTSQP | RRIKVIT | NEQSTFFEV | PVLSTRVPSR | NNYLSRFQ | HLENQLKLN | FSHFAEDI |
| | | FGFHYL | FVEKRL | KHSSQIPKPD | INEQNTLTQ | SGSTWGKQ | NFGVLRLALS | WFDTMEQ |
| | | FNNRKC | KGGWT | FRERFEEKQR | FLVDLDCKS | PVLSTRVPS | HSKAQFSPE | HEAIAGAFP |
| | | RYTPDF | EKNLNP | VALSEFNRRL | GGRGIRRRN | RINEQNTLT | YKAVHRLGS | LELNRINIY |
| | | LAIGHN | ILSLVES | VLVTEKQIR | EIALGEAVVK | QFLVDLDC | DYPFVFLGKR | HAQTTSQ |
| | | EQSTFF | ELQLTP | MGPTLDNFK | QLKRKSVELII | KSGGRGIRR | FTPICPLCISE | MRVRVLIH |
| | | EVKHSS | PSWRT | LLHRYSGLRT | VNEIQELVEF | RNEIALGEA | APYIRQQW | LENQLKLN |
| | | QIPKPD | VATWK | VTEFQKRVL | STAEQRQVI | VVKQLKRK | QFLSQQACE | NFGVLRLAL |
| | | FRERFE | KSYAEA | AFIQRKQMV | ANTFKYMSE | SVELIIVNEI | RHGCKLVHH | SHSKAQFSP |
| | | EKQRVA | GREASA | KLQEVSLYFG | EARVSFVLV | QELVEFSTA | CPECQSRLEY | EYKAVHRL |
| | | LSEFNR | LIPKHTF | LSEQDTLISTL | GMPYADVIA | EQRQVIAN | QTTESISQCE | GSDYPFVFL |
| | | RLVLVT | KGNRQ | PWISSGHVK | TEPQWNSRL | TFKYMSEE | CGFELRNSP | GKRFTPICP |
| | | EKQIRM | KEMDS | TDLNTIGFGL | SWRRKIDYF | ARVSFVLV | VEDAPVAAL | LCISEAPYIR |
| | | GPTLDN | QSLIDE | ETCVWCGS | KLLKANSHSS | GMPYADVI | LVARWLSGN | QQWQFLS |
| | | FKLLHR | AIQNVY | GPKKKRKVG | KTASYGFDLE | ATEPQWNS | DSKPLGLLKA | QQACERHG |
| | | YSGLRT | LTRERLS | SGYPYDVPD | QKKHFARFV | RLSWRRKI | EMTLSERYG | CKLVHHCP |
| | | VTEFQK | VAEAYR | YAYPYDVPD | VGLSSRMGF | DYFKLLKAN | FLLWYVNRY | ECQSRLEYQ |
| | | RVLAFI | YYKSRVI | YAYPYDVPD | DEPPVLTKN | SHSSKTASY | GDIENISFESF | TTESISQCE |
| | | QRKQM | QMNRG | YAGSGPPDS | ELLYPLFAMC | GFDLEQKK | VEYCSCWPR | CGFELRNSP |
| | | VKLQEV | IVEGKIK | NSIFGFFDEF | RGECRALKH | HFARFVVG | VLKEELDELV | VEDAPVAA |
| | | SLYFGLS | PIAERSF | EASEEESQLL | FLKDALLTSF | LSSRMGFD | NKADLIRIKD | LLVARWLS |
| | | EQDTLIS | YNRINE | PKELILEPVEI | NDNADTIDK | EPPVLTKNE | WKKTFFNEV | GNDSKPLG |
| | | TLPWIS | LPPYEV | SSTIDSLPAKI | AILSRTFAFKF | LLYPLFAMC | FGALLKDCR | LLKAEMTLS |
| | | SGHVKT | AIARFG | QEEVLRRIKV | PYLDNPFDR | RGECRALK | QLPSRQLEC | ERYGFLLW |
| | | DLNTIG | KRYADR | ITFVEKRLKG | PLEQLSLHQI | HFLKDALLT | NSVLTQVLA | YVNRYGDIE |
| | | FGLETC | EYRSVG | GWTEKNLN | DSGSAYHLN | SFNDNADTI | YFTKLMAAIP | NISFESFVEY |
| | | VWC | QQVVA | PILSLVESELQ | AITTEDKIVA | DKAILSRTF | SSSKGNVGD | CSCWPRVL |
| | | (SEQ ID | TKPMEF | LTPPSWRTV | PRFTDAIPLS | AFKFPYLDN | VLLSPLEAST | KEELDELVN |
| | | NO: 176) | VEIDHT | ATWKKSYAE | MLLSKNGLK | PFDRPLEQL | LLSCTTDEVY | KADLIRIKD |
| | | | PVPVILI | AGREASALIP | A (SEQ ID | SLHQIDSGS | RLYEFGEIKA | WKKTFFNE |
| | | | DDELDI | KHTFKGNRQ | NO: 179) | GSGPAAKK | AIRPRMHTKI | VFGALLKDC |
| | | | PLGRPY | KEMDSQSLI | | KKLDGSGA | ASHESAFTLR | RQLPSRQLE |
| | | | LTMLYD | DEAIQNVYLT | | YHLNAITTE | SVIETKLTRM | CNSVLTQV |
| | | | RFSKCIV | RERLSVAEAY | | DKIVAPRFT | CSENDGLSV | LAYFTKLM |
| | | | GCSINF | RYYKSRVIQ | | DAIPLSMLL | YLPEW (SEQ | AAIPSSSKG |
| | | | REPSFD | MNRGIVEGK | | SKNGLKA* | ID NO: 181) | NVGDVLLS |
| | | | SVRKAL | IKPIAERSFYN | | (SEQ ID | | PLEASTLLS |
| | | | LNSLLD | RINELPPYEV | | NO: 180) | | CTTDEVYRL |
| | | | KSWLKA | AIARFGKRYA | | | | YEFGEIKAAI |
| | | | KYPSIEN | DREYRSVGQ | | | | RPRMHTKI |
| | | | EWPCH | QVVATKPM | | | | ASHESAFTL |
| | | | GKIDCL | EFVEIDHTPV | | | | RSVIETKLTR |
| | | | VVDNG | PVILIDDELDI | | | | MCSENDGL |
| | | | AEFWS | PLGRPYLTM | | | | SVYLPEW |
| | | | QSLEDS | LYDRFSKCIV | | | | (SEQ ID |
| | | | LRPLVS | GCSINFREPS | | | | NO: 182) |
| | | | DIQYSQ | FDSVRKALL | | | | |
| | | | AAKPW | NSLLDKSWL | | | | |
| | | | RKSGIEK | KAKYPSIENE | | | | |
| | | | LFDQM | WPCHGKIDC | | | | |
| | | | NKGLV | LVVDNGAEF | | | | |
| | | | NALPGK | WSQSLEDSL | | | | |
| | | | TFTNPT | RPLVSDIQYS | | | | |
| | | | QLQDY | QAAKPWRK | | | | |
| | | | NPKKDA | SGIEKLFDQ | | | | |
| | | | VVRVSV | MNKGLVNA | | | | |
| | | | FLELLHK | LPGKTFTNPT | | | | |
| | | | WIVDYY | QLQDYNPKK | | | | |
| | | | HMAPD | DAVVRVSVF | | | | |
| | | | SREREIP | LELLHKWIVD | | | | |
| | | | YHKWH | YYHMAPDSR | | | | |
| | | | QSKWT | EREIPYHKW | | | | |
| | | | PSYYDG | HQSKWTPSY | | | | |
| | | | AEKEQL | YDGAEKEQL | | | | |
| | | | RVELGL | RVELGLLRHR | | | | |
| | | | LRHRTI | TIGVAGIRLH | | | | |
| | | | GVAGIR | NLRYQSAELI | | | | |
| | | | LHNLRY | EYRKYCTPN | | | | |
| | | | QSAELIE | NGKQLFVKT | | | | |
| | | | YRKYCT | KTDPSDISYI | | | | |
| | | | PNNGK | HVYLESEKKY | | | | |
| | | | QLFVKT | IKVPAVDNS | | | | |
| | | | KTDPSD | GYTNGLSLFE | | | | |
| | | | ISYIHVY | HQRIQKVRR | | | | |
| | | | LESEKKY | LNTKDLADD | | | | |
| | | | IKVPAV | EALADTFLY | | | | |
| | | | DNSGYT | MKKRIHEET | | | | |
| | | | NGLSLF | DRFRRVKSS | | | | |
| | | | EHQRIQ | KPNLPKTGN | | | | |
| | | | KVRRLN | TSRLAKFND | | | | |
| | | | TKDLAD | VGSEGPNSI | | | | |
| | | | DEALAD | NVTPVRLKSE | | | | |
| | | | TFLYMK | VVSDASEYL | | | | |
| | | | KRIHEET | DDDDFEDIE | | | | |
| | | | DRFRRV | GY* (SEQ ID | | | | |
| | | | KSSKPN | NO: 178) | | | | |
| | | | LPKTGN | | | | | |
| | | | TSRLAK | | | | | |
| | | | FNDVGS | | | | | |
| | | | EGPNSI | | | | | |
| | | | NVTPVR | | | | | |
| | | | LKSEVV | | | | | |
| | | | SDASEY | | | | | |
| | | | LDDDDF | | | | | |
| | | | EDIEGY | | | | | |
| | | | (SEQ ID | | | | | |
| | | | NO: 177) | | | | | |
| 28 | V. | MYIRNL | MVGRF | MYIRNLRKP | MGRAQKSK | MGRAQKS | VETDIQLYPD | MPKKKRKV |
| | hyu- | RKPSPN | HDEFEP | SPNKNIFKFS | EIVVTAARR | KEIVVTAAR | ESLESFLLRLS | GSGEQKLIS |
| | gaensis_ | KNIFKFS | EYDEDS | SLKNRDAVM | NLNRDEVLA | RNLNRDEV | QEQGYERFS | EEDLEQKLI |
| | 151112A_ | SLKNRD | DLKHEF | CEGSLEKDC | NYHDSFSIYP | LANYHDSFS | HFAEDIWFD | SEEDLEQKL |
| | GCA_0008 | AVMCE | LPAAQT | CYHFEYDPD | EVEKVLSGLE | IYPEVEKVLS | TLNQHEAIA | ISEEDLGSG |
| | 18475.1 | GSLEKD | ESLKYSR | VVRYESQPE | WIIKRRKFGT | GLEWIIKRR | GAFPLELNR | ETDIQLYPD |
| | | CCYHFE | LQSTQII | GFYYDFNGK | FAPSMLLTA | KFGTFAPS | VNIYHAQTT | ESLESFLLRL |
| | | YDPDVV | ERDLSS | KRPYTPDFLV | GTGAGKTAT | MLLTAGTG | SQMRVRVLI | SQEQGYER |
| | | RYESQP | YPEEQK | TYHDGTFEY | INHFIEKNLS | AGKTATIN | HLENQLKLN | FSHFAEDI |
| | | EGFYYD | NKALER | VEVKPHTKTL | RNEVLITRVR | HFIEKNLSR | NFGVLRLALS | WFDTLNQ |
| | | FNGKKR | YKLLCLV | SKTFKQEFSA | PSLLETLLW | NEVLITRVR | HSKAQFSSQ | HEAIAGAFP |
| | | PYTPDF | ANELNG | RKEAANRRG | MAKELGAYR | PSLLETLLW | YKAVHRFGS | LELNRVNIY |
| | | LVTYHD | GWTSK | VSLVLVTDK | NSRAKPSEIG | MAKELGAY | DYPYAFLRKR | HAQTTSQ |
| | | GTFEYV | NLTPLIE | QIRDGYFLK | LTDCVIETSK | RNSRAKPSE | FTPICPLCVD | MRVRVLIH |
| | | EVKPHT | KHFDKT | NTELVHRYS | RVGLKLLVIE | IGLTDCVIET | EAPYIRQQW | LENQLKLN |
| | | KTLSKTF | CLPKKP | GCIAGDELAI | ECQELFERTS | SKRVGLKLL | QLISHQACE | NFGVLRLAL |
| | | KQEFSA | SYKSLQ | KVYSYLIAQN | HNQRQDIRD | VIEECQELF | HHGCKLVHH | SHSKAQFSS |
| | | RKEAAN | RWHNS | TMKISDLAD | RLKMISDEC | ERTSHNQR | CPECKSRLEY | QYKAVHRF |
| | | RRGVSL | FVDSDG | SIGESVGRVF | HLPIVFVGLH | QDIRDRLK | QSTESISQCE | GSDYPYAFL |
| | | VLVTDK | SFTSLV | ASVLRLIAVG | SAGLILEDSQ | MISDECHLP | CGYELRNSP | RKRFTPICP |
| | | QIRDGY | DKNHLK | KAGVDLDIA | WNRRIMVR | IVFVGLHSA | VEDAPEAEV | LCVDEAPYI |
| | | FLKNTE | GNRDA | QLSESTTVSV | RTLPYIKITDE | GLILEDSQ | LVARWLSGN | RQQWQLIS |
| | | LVHRYS | RVVGDE | RGSGPKKKR | SAIDNYLDVL | WNRRIMV | DSKPLGLLTG | HQACEHH |
| | | GCIAGD | KYYDEA | KVGSGYPYD | QALEKTVPLP | RRTLPYIKIT | EMTLSERYG | GCKLVHHC |
| | | ELAIKVY | LKMFLD | VPDYAYPYD | FKVPLTDVD | DESAIDNYL | FLLWYINRY | PECKSRLEY |
| | | SYLIAQ | ARRQSI | VPDYAYPYD | FAMRLLSAS | DVLQALEK | GDIDDLSFES | QSTESISQC |
| | | NTMKIS | RAAHAF | VPDYAGSGV | KGILGEIKELI | TVPLPFKVP | FIEYCCAWP | ECGYELRNS |
| | | DLADSI | YCDRIT | GRFHDEFEP | AAALDVALA | LTDVDFAM | TALWQDLD | PVEDAPEA |
| | | GESVGR | VANEAI | EYDEDSDLK | KNKDYIGEE | RLLSASKGIL | ALKEKAELVR | EVLVARWL |
| | | VFASVL | VAGRIP | HEFLPAAQT | DFAAVYEKIN | GEIKELIAA | VKDWKKMF | SGNDSKPL |
| | | RLIAVG | KVSYEA | ESLKYSRLQS | DPNDINPFT | ALDVALAK | FNEAFDTLLK | GLLTGEMT |
| | | KAGVDL | FKKRIRK | TQIIERDLSSY | VQIDALTIEQ | NKDYIGEED | GCRQLPSRQ | LSERYGFLL |
| | | DIAQLS | EEPYSV | PEEQKNKAL | IASYENYVTD | FAAVYEKIN | LSHNTVLTQ | WYINRYGD |
| | | ESTTVS | VLARHG | ERYKLLCLVA | AETGELRFVK | DPNDINPFT | VLAYFTQLM | IDDLSFESFI |
| | | VR (SEQ | KYYADK | NELNGGWT | QVFSKLSIQQ | VQIDALTIE | ATVPSSAKG | EYCCAWPT |
| | | ID | LFNYYQ | SKNLTPLIEK | LIG (SEQ ID | QIASYEGSG | NIGDALLSPL | ALWQDLD |
| | | NO: 183) | SVEMPT | HFDKTCLPKK | NO: 186) | PAAKKKKL | EASTLLSCTT | ALKEKAELV |
| | | | RILERVE | PSYKSLQRW | | DGSGNYVT | DEVYRLYEFG | RVKDWKK |
| | | | MDHTP | HNSFVDSDG | | DAETGELRF | EIKAAIRPRM | MFFNEAFD |
| | | | LDLILLH | SFTSLVDKN | | VKQVFSKLS | HTKIASHESA | TLLKGCRQL |
| | | | DELMV | HLKGNRDAR | | IQQLIG* | FTLRSVIETKL | PSRQLSHN |
| | | | PLGRAH | VVGDEKYYD | | (SEQ ID | TRMSSESDG | TVLTQVLAY |
| | | | LTLLVD | EALKMFLDA | | NO: 187) | LSVYLPEW | FTQLMATV |
| | | | VFSGCII | RRQSIRAAH | | | (SEQ ID | PSSAKGNIG |
| | | | GFHLGF | AFYCDRITVA | | | NO: 188) | DALLSPLEA |
| | | | KAPSYV | NEAIVAGRIP | | | | STLLSCTTD |
| | | | SASRAV | KVSYEAFKKR | | | | EVYRLYEFG |
| | | | HATKS | IRKEEPYSVV | | | | EIKAAIRPR |
| | | | KSYISE | LARHGKYYA | | | | MHTKIASH |
| | | | MPISFN | DKLFNYYQS | | | | ESAFTLRSVI |
| | | | NEWLC | VEMPTRILER | | | | ETKLTRMSS |
| | | | EGKIEN | VEMDHTPLD | | | | ESDGLSVYL |
| | | | LVVDN | LILLHDELMV | | | | PEW (SEQ |
| | | | GAEFW | PLGRAHLTLL | | | | ID NO: 189) |
| | | | SKSWE | VDVFSGCIIG | | | | |
| | | | DACLEV | FHLGFKAPSY | | | | |
| | | | GINVVY | VSASRAVIHA | | | | |
| | | | NKVRKP | TKSKSYISEM | | | | |
| | | | WLKPFI | PISFNNEWL | | | | |
| | | | ERKFGEI | CEGKIENLVV | | | | |
| | | | VQGIVG | DNGAEFWS | | | | |
| | | | WVPGK | KSWEDACLE | | | | |
| | | | TFSNVL | VGINVVYNK | | | | |
| | | | EKEDYK | VRKPWLKPFI | | | | |
| | | | PEKDAV | ERKFGEIVQ | | | | |
| | | | MRFSTF | GIVGWVPGK | | | | |
| | | | VEEFHR | TFSNVLEKED | | | | |
| | | | WIVDV | YKPEKDAVM | | | | |
| | | | HNANA | RFSTFVEEFH | | | | |
| | | | DSRYKRI | RWIVDVHN | | | | |
| | | | PNLYW | ANADSRYKR | | | | |
| | | | KQSYDA | IPNLYWKQS | | | | |
| | | | LPPLKLL | YDALPPLKLL | | | | |
| | | | PEHEQA | PEHEQAFRV | | | | |
| | | | FRVVM | VMGILQYRK | | | | |
| | | | GILQYR | LTDKGIKFM | | | | |
| | | | KLTDKG | HLEYDCVALS | | | | |
| | | | IKFMHL | DYRKTYPQT | | | | |
| | | | EYDCVA | NESSKKKIKV | | | | |
| | | | LSDYRK | DPDDLSAIYV | | | | |
| | | | TYPQTN | YLDELQGYV | | | | |
| | | | ESSKKKI | KVPSKDPMG | | | | |
| | | | KVDPD | YTVRLSVCEH | | | | |
| | | | DLSAIYV | EKILAAHRTY | | | | |
| | | | YLDELQ | IKGEMDVLS | | | | |
| | | | GYVKVP | LAKARLALH | | | | |
| | | | SKDPM | DRIESEQADL | | | | |
| | | | GYTVRL | MQLTHTERK | | | | |
| | | | SVCEHE | RKAKSTKKV | | | | |
| | | | KILAAH | AEISSVNSDT | | | | |
| | | | RTYIKGE | PHSKLSDRTP | | | | |
| | | | MDVLSL | KPNKKVAES | | | | |
| | | | AKARLA | EKSSDTTPLE | | | | |
| | | | LHDRIES | SFRAKWDER | | | | |
| | | | EQADL | RNLRK* | | | | |
| | | | MQLTH | (SEQ ID | | | | |
| | | | TERKRK | NO: 185) | | | | |
| | | | AKSTKK | | | | | |
| | | | VAEISSV | | | | | |
| | | | NSDTPH | | | | | |
| | | | SKLSDR | | | | | |
| | | | TPKPNK | | | | | |
| | | | KVAESE | | | | | |
| | | | KSSDTT | | | | | |
| | | | PLESFR | | | | | |
| | | | AKWDE | | | | | |
| | | | RRNLRK | | | | | |
| | | | (SEQ ID | | | | | |
| | | | NO: 184) | | | | | |
| 29 | V. | MYDQT | MSDDL | MYDQTKKSS | LVNILSELQIE | MNILSELQI | MDQHEAIA | MPKKKRKV |
| | crass- | KKSSAV | FGFSDE | AVHNICKFM | QYTSFRECFL | EQYTSFREC | GAFPLDLNL | GSGEQKLIS |
| | ostreae_ | HNICKF | FNSFDN | SLKNDSVVR | EYPQLTEIYN | FLEYPQLTEI | VNIYHAQTT | EEDLEQKLI |
| | J5_20_ | MSLKN | DVADD | TMSMLEYDF | VFDRMVLNS | YNVFDRMV | SQMRVRVLI | SEEDLEQKL |
| | GCA_0010 | DSVVRT | KTLSTEF | CFHAEYNPQ | SLGGEQESLL | LNSSLGGE | HLENQLKLN | ISEEDLGSG |
| | 48515.1 | MSMLE | LAEYEN | IVRYESQPH | LTGDTGVGK | QESLLLTGD | NFGVLRLALS | DQHEAIAG |
| | | YDFCFH | LELAFG | GFEYYFNGR | TAMIDNYVA | TGVGKTAM | HSKAQFSPQ | AFPLDLNLV |
| | | AEYNPQ | DLPNKE | YCRYTPDFQ | RFAIKGSRW | IDNYVARFA | YKAVHRFGS | NIYHAQTTS |
| | | IVRYES | TALFRL | LFDSIDTPSLI | AEMPVLKTR | IKGSRWAE | DYPYAFLRKR | QMRVRVLI |
| | | QPHGFE | DLIRYLE | EVKHSSQILK | IPSKVREQNT | MPVLKTRIP | FTPICPLCIDE | HLENQLKL |
| | | YYFNGR | RRVKG | PDFRARFKE | LERLLIDLDSR | SKVREQNT | APYIRQQW | NNFGVLRL |
| | | YCRYTP | GWTPK | KQLVAQAEY | ASSRRRRPYK | LERLLIDLDS | QFISHQACE | ALSHSKAQ |
| | | DFQLFD | NLDKLL | GKKLILVTEK | EGALEQGVI | RASSRRRRP | HHGCKLIHH | FSPQYKAV |
| | | SIDTPSL | EEYALLK | QIRTGFLLSN | KSLIEKKVKL | YKEGALEQ | CPECKLRLEY | HRFGSDYP |
| | | IEVKHSS | KTSVPS | LKLLHGYSGI | VIVNEVQEL | GVIKSLIEKK | QSTESSSQCE | YAFLRKRFT |
| | | QILKPD | SRTIAD | RTITDIQKHV | MEFKDANER | VKLVIVNEV | CGFELRNSP | PICPLCIDEA |
| | | FRARFK | WKKLYY | LQFVQANRS | QTIANTFKMI | QELMEFKD | VEGAPEVEV | PYIRQQWQ |
| | | EKQLVA | ESGKDL | VTLHHLAHQ | SEEAQVSFVL | ANERQTIA | LVAQWLSG | FISHQACEH |
| | | QAEYGK | ASLIPG | LKISPDETLT | VGMPYATM | NTFKMISEE | NDSKPLGLLK | HGCKLIHHC |
| | | KLILVTE | HSKKGN | AALCWLSSG | LAEEDQWN | AQVSFVLV | GEMTLSERY | PECKLRLEY |
| | | KQIRTG | RKLKND | EIQTDFNQK | SRLGWKRHL | GMPYATM | GFLLWYVNR | QSTESSSQC |
| | | FLLSNLK | SSDLVT | KFDLENSVW | SYFHLSKLSE | LAEEDQW | HGDIDDLSFE | ECGFELRNS |
| | | LLHGYS | EAIQTK | CGSGPKKKR | ADKKGYIPD | NSRLGWKR | SFIEYCGSWP | PVEGAPEV |
| | | GIRTITD | FLTKER | KVGSGYPYD | AEGKRHFAS | HLSYFHLSK | TALWQDLD | EVLVAQWL |
| | | IQKHVL | VSVNTA | VPDYAYPYD | FVAGLAGR | LSEADKKGY | ALKEKAELIR | SGNDSKPL |
| | | QFVQA | YEYYKY | VPDYAYPYD | MGFEKRPNL | IPDAEGKRH | VKDWKKMF | GLLKGEMT |
| | | NRSVTL | RVIEEN | VPDYAGSGS | TGDEILLPLFS | FASFVAGLA | FNEAFGALLK | LSERYGFLL |
| | | HHLAH | RQLDQ | DDLFGFSDE | VCRGECRVL | GRMGFEKR | DCRQLPSRQ | WYVNRHG |
| | | QLKISP | VKIAPIS | FNSFDNDVA | KHFLADALL | PNLTGDEIL | LSHNIVLTRV | DIDDLSFES |
| | | DETLTA | QRTFYN | DDKTLSTEFL | NALQSSKDTI | LPLFSVCRG | LAYFAKLMA | FIEYCGSWP |
| | | ALCWLS | RVNALP | AEYENLELAF | DKPLLSACFD | ECRVLKHFL | TVPSSAKGNI | TALWQDLD |
| | | SGEIQT | PYEVAL | GDLPNKETA | TKYPYAKQN | ADALLNAL | GDVLLSPLEA | ALKEKAELI |
| | | DFNQK | ARYGKR | LFRLDLIRYLE | PFECKLTELK | QSSKDTIDK | STLLSCTTDE | RVKDWKK |
| | | KFDLEN | YADNKF | RRVKGGWT | LVELKTETSY | PLLSACFDT | VYRLYEFGEI | MFFNEAFG |
| | | SVWC | KTVGSII | PKNLDKLLEE | NKGAQFKED | KYPYAKQN | KAAIRPRMH | ALLKDCRQL |
| | | (SEQ ID | PATRP | YALLKKTSVP | RLIGRSFTDL | PFECKLTEL | TKIASHESAF | PSRQLSHNI |
| | | NO: 190) | MEYVEI | SSRTIADWK | LPVHMLLSK | KLVELKTET | TLRSVIETKLI | VLTRVLAYF |
| | | | DHTTAP | KLYYESGKDL | TPLKAQ | GSGPAAKK | RMSSESDGL | AKLMATVP |
| | | | VILLDD | ASLIPGHSKK | (SEQ ID | KKLDGSGSY | SVYLPEW | SSAKGNIG |
| | | | DLELPL | GNRKLKNDS | NO: 193) | NKGAQFKE | (SEQ ID | DVLLSPLEA |
| | | | GRPHLT | SDLVTEAIQT | | DRLIGRSFT | NO: 195) | STLLSCTTD |
| | | | ILYDRYS | KFLTKERVSV | | DLLPVHML | | EVYRLYEFG |
| | | | TCIVGLS | NTAYEYYKYR | | LSKTPLKAQ | | EIKAAIRPR |
| | | | VNYRDP | VIEENRQLD | | * (SEQ ID | | MHTKIASH |
| | | | SYETVR | QVKIAPISQR | | NO: 194) | | ESAFTLRSVI |
| | | | AAFLNS | TFYNRVNAL | | | | ETKLIRMSS |
| | | | VLKKD | PPYEVALARY | | | | ESDGLSVYL |
| | | | WIKEKY | GKRYADNKF | | | | PEW (SEQ |
| | | | PSIESD | KTVGSIIPAT | | | | ID NO: 196) |
| | | | WPCYG | RPMEYVEID | | | | |
| | | | KITNLIV | HTTAPVILLD | | | | |
| | | | DNGAEF | DDLELPLGRP | | | | |
| | | | WSDSLE | HLTILYDRYS | | | | |
| | | | SALKPL | TCIVGLSVNY | | | | |
| | | | VTDIQY | RDPSYETVR | | | | |
| | | | NQRGK | AAFLNSVLKK | | | | |
| | | | PWRKA | DWIKEKYPSI | | | | |
| | | | GVEKSF | ESDWPCYGK | | | | |
| | | | DTFYKK | ITNLIVDNGA | | | | |
| | | | LFSRFP | EFWSDSLES | | | | |
| | | | GKTFTN | ALKPLVTDIQ | | | | |
| | | | PTQLKD | YNQRGKPW | | | | |
| | | | YNPKQ | RKAGVEKSF | | | | |
| | | | DAVINV | DTFYKKLFSR | | | | |
| | | | SDFLELL | FPGKTFTNPT | | | | |
| | | | HKWLID | QLKDYNPKQ | | | | |
| | | | VYHKKA | DAVINVSDFL | | | | |
| | | | DTRYKR | ELLHKWLIDV | | | | |
| | | | VPYQK | YHKKADTRY | | | | |
| | | | WTESQ | KRVPYQKWT | | | | |
| | | | GTIIFCE | ESQGTIIFCE | | | | |
| | | | GPEAEQ | GPEAEQLKIE | | | | |
| | | | LKIELGA | LGAVNHRTI | | | | |
| | | | VNHRTI | RRGAIELYSL | | | | |
| | | | RRGAIE | KYQSDELEEY | | | | |
| | | | LYSLKY | GKQYSSRAR | | | | |
| | | | QSDELE | KSAYVKIKTD | | | | |
| | | | EYGKQY | PNDISSIYVYL | | | | |
| | | | SSRARK | EEEKRYIKVP | | | | |
| | | | SAYVKIK | AVDHTGYTK | | | | |
| | | | TDPNDI | GRSLYEHQRI | | | | |
| | | | SSIYVYL | NSLRRLKVRL | | | | |
| | | | EEEKRYI | GEQDESLAD | | | | |
| | | | KVPAVD | ASLYLDRAM | | | | |
| | | | HTGYTK | DEAIERMSR | | | | |
| | | | GRSLYE | SKSKKSALPK | | | | |
| | | | HQRINS | TTHASKIAKQ | | | | |
| | | | LRRLKV | RGVGSEGPS | | | | |
| | | | RLGEQD | TIVTTSPKPII | | | | |
| | | | ESLADA | EVPKEVIDM | | | | |
| | | | SLYLDR | GTTSDDLSDI | | | | |
| | | | AMDEAI | EGY* (SEQ | | | | |
| | | | ERMSRS | ID NO: 192) | | | | |
| | | | KSKKSA | | | | | |
| | | | LPKTTH | | | | | |
| | | | ASKIAK | | | | | |
| | | | QRGVG | | | | | |
| | | | SEGPSTI | | | | | |
| | | | VTTSPK | | | | | |
| | | | PIIEVPK | | | | | |
| | | | EVIDMG | | | | | |
| | | | TTSDDL | | | | | |
| | | | SDIEGY | | | | | |
| | | | (SEQ ID | | | | | |
| | | | NO: 191) | | | | | |
| 30 | A. | MYRRH | MCAQP | MYRRHLKHS | MELSSTDAD | MELSSTDA | MQLLVRPAP | MPKKKRKV |
| | sal- | LKHSRV | TTEVPS | RVKNLFKFVS | KLKSFIECYVE | DKLKSFIECY | FSDESLESYLL | GSGEQKLIS |
| | monicida | KNLFKF | DLFEDE | AKMNTVFTV | TPLLRIIQDD | VETPLLRIIQ | RLSQENGFE | EEDLEQKLI |
| | strain | VSAKM | FTHPHP | ESSLEFDTCF | FDRLRYDKQ | DDFDRLRY | RYALLSGAM | SEEDLEQKL |
| | AJ83 | NTVFTV | PESPNL | HLEYSPAVK | FAGEPICMLL | DKQFAGEPI | RDALLQQDH | ISEEDLGSG |
| | | ESSLEFD | AATTPT | AFEAQPEGF | TGDSGTGKS | CMLLTGDS | QAAGAFPLE | QLLVRPAPF |
| | | TCFHLE | VLSATV | YYTFEGRDC | SLIRHYMAQ | GTGKSSLIR | LARVNVFHA | SDESLESYLL |
| | | YSPAVK | DSFPAD | PYTPDFRVL | FPEQHGHGF | HYMAQFPE | NRSSSLRVRA | RLSQENGF |
| | | AFEAQP | LKAQAL | NENGSVGYL | VRKPLLVSRI | QHGHGFVR | LHLIEQLTDL | ERYALLSGA |
| | | EGFYYT | HRLDYI | EVKPSAKVLE | PSKPTLESTM | KPLLVSRIPS | APHSLLQLAL | MRDALLQ |
| | | FEGRDC | RWIED | SDFLQRFPFK | VELLKDLGQ | KPTLESTM | IRSAMPIGA | QDHQAAG |
| | | PYTPDF | NLAGG | QQRATELSC | WGSEYRLHR | VELLKDLGQ | GHACVQRG | AFPLELARV |
| | | RVLNEN | WTEKN | PLKLITERQIR | SSAESLTEALI | WGSEYRLH | GVDIPLRLVR | NVFHANRS |
| | | GSVGYL | LAPLLVE | IDPILGNLKLL | KCLTRCETELI | RSSAESLTE | TRQIPVCPVC | SSLRVRALH |
| | | EVKPSA | AAKVLP | HRYSGFQSF | IIDEFQELIEN | ALIKCLTRC | LSESAYIRQH | LIEQLTDLA |
| | | KVLESD | PPAPN | TPLHMQLLG | KTREKRNQI | ETELIIIDEF | WHYAPYVA | PHSLLQLAL |
| | | FLQRFP | WRTLA | LVKDFGRVSL | ANRLKYISET | QELIENKTR | CHLHGHELL | IRSAMPIGA |
| | | FKQQR | RWQKN | ARLSGSTGA | AKIPIVLVGM | EKRNQIAN | SVCPSCGKAL | GHACVQR |
| | | ATELSC | YNQHG | PPGEVLATVL | PWAAKIAEE | RLKYISETAK | DYQCNESFT | GGVDIPLRL |
| | | PLKLITE | RKLMAL | SLIARGLIHS | PQWASRLM | IPIVLVGMP | HCRCGFDLR | VRTRQIPVC |
| | | RQIRIDP | IPKHQA | DLAEHEMGF | VQRTIPFFKL | WAAKIAEE | HSITPPASNQ | PVCLSESAYI |
| | | ILGNLKL | KGNVKS | STIVWMRGS | SEDAESFVRF | PQWASRL | AIQISALICGA | RQHWHYA |
| | | LHRYSG | RLPSSD | GPKKKRKVG | VMGLARRM | MVQRTIPF | RWESTNPLLI | PYVACHLH |
| | | FQSFTP | EVFFEQ | SGYPYDVPD | PFATPPKLEA | FKLSEDAES | CPHPSQLFG | GHELLSVCP |
| | | LHMQLL | AVHCFL | YAYPYDVPD | KHTIFALFAF | FVRFVMGL | AIFWYWCRY | SCGKALDY |
| | | GLVKDF | VGEQPS | YAYPYDVPD | SYGCVRRLK | ARRMPFAT | HAEAAGQP | QCNESFTH |
| | | GRVSLA | IASVYQ | YAGSGCAQP | HLLDESVKQ | PPKLEAKHT | ASHSLVQTID | CRCGFDLR |
| | | RLSGST | YYTDIICI | TTEVPSDLFE | ALAAHSETLL | IFALFAFSY | YFAAWPANF | HSITPPASN |
| | | GAPPGE | ENLNVV | DEFTHPHPP | HEHIAVAFG | GCVRRLKH | HAELDQWA | QAIQISALIC |
| | | VLATVL | ENPIKAI | ESPNLAATTP | LFYPDQENP | LLDESVKQA | QRGLLRQTR | GARWESTN |
| | | SLIARGL | SYTAFF | TVLSATVDSF | FLQSIDEIKA | LAAHSETLL | LLNETPFGEV | PLLICPHPS |
| | | IHSDLA | NRLKKL | PADLKAQAL | CEVTQYSRYE | HEHIAVAF | FGAVLSDCR | QLFGAIFW |
| | | EHEMG | PAYQVI | HRLDYIRWIE | INESGTEEVL | GLFYPDQE | QLPFQDLGA | YWCRYHAE |
| | | FSTIVW | KSRKGS | DNLAGGWT | NPLKFTDKIPI | NPFLQSIDE | NFILRALSDY | AAGQPASH |
| | | MR | YMADV | EKNLAPLLVE | SQLLKKR | IKACEVTQY | LTALVVNHP | SLVQTIDYF |
| | | (SEQ ID | EFMAIS | AAKVLPPPA | (SEQ ID | SGSGPAAK | KTRQPNLGD | AAWPANF |
| | | NO: 197) | SHIPPSC | PNWRTLAR | NO: 200) | KKKLDGSG | ILLSASDAAA | HAELDQW |
| | | | VMERV | WQKNYNQH | | RYEINESGT | LLSTSVEQVF | AQRGLLRQ |
| | | | EIDHTPL | GRKLMALIP | | EEVLNPLKF | RLQQEGYLT | TRLLNETPF |
| | | | DLILLDD | KHQAKGNV | | TDKIPISQLL | LAYRLRRHA | GEVFGAVL |
| | | | DLLVPL | KSRLPSSDEV | | KKR* (SEQ | GLTPYDPMF | SDCRQLPF |
| | | | GRPCLT | FFEQAVHCF | | ID NO: 201) | HLRQVIEYRL | QDLGANFIL |
| | | | LLIDSYS | LVGEQPSIAS | | | AHGAMYPP | RALSDYLTA |
| | | | HCVVGF | VYQYYTDIICI | | | AFYSFLPAW | LVVNHPKT |
| | | | NLSFNQ | ENLNVVENP | | | (SEQ ID | RQPNLGDIL |
| | | | PGYESV | IKAISYTAFFN | | | NO: 202) | LSASDAAAL |
| | | | RNALLN | RLKKLPAYQ | | | | LSTSVEQVF |
| | | | SIPPKNY | VIKSRKGSY | | | | RLQQEGYL |
| | | | VKDKYP | MADVEFMA | | | | TLAYRLRRH |
| | | | SVEHE | ISSHIPPSCV | | | | AGLTPYDP |
| | | | WPCYG | MERVEIDHT | | | | MFHLRQVI |
| | | | KPATLV | PLDLILLDDD | | | | EYRLAHGA |
| | | | VDNGV | LLVPLGRPCL | | | | MYPPAFYS |
| | | | EFWSKS | TLLIDSYSHC | | | | FLPAW |
| | | | LEQSCR | VVGFNLSFN | | | | (SEQ ID |
| | | | ELNINT | QPGYESVRN | | | | NO: 203) |
| | | | QYNPV | ALLNSIPPKN | | | | |
| | | | RKPWLK | YVKDKYPSV | | | | |
| | | | PMVER | EHEWPCYGK | | | | |
| | | | MFGTIN | PATLVVDNG | | | | |
| | | | RKLLESI | VEFWSKSLE | | | | |
| | | | PGKTFS | QSCRELNINT | | | | |
| | | | NLLERG | QYNPVRKP | | | | |
| | | | EYDPQK | WLKPMVER | | | | |
| | | | DAVMR | MFGTINRKL | | | | |
| | | | FSTFLEI | LESIPGKTFS | | | | |
| | | | FHRWII | NLLERGEYD | | | | |
| | | | DVYHYE | PQKDAVMR | | | | |
| | | | PDSRRR | FSTFLEIFHR | | | | |
| | | | YIPIQS | WIIDVYHYEP | | | | |
| | | | WQYGC | DSRRRYIPIQ | | | | |
| | | | NKLPPA | SWQYGCNK | | | | |
| | | | PVVGD | LPPAPVVGD | | | | |
| | | | DLAKLE | DLAKLEVILSI | | | | |
| | | | VILSISL | SLQCTHRRG | | | | |
| | | | QCTHRR | GIQRFHLRY | | | | |
| | | | GGIQRF | DSDELASYR | | | | |
| | | | HLRYDS | MNYPDKTH | | | | |
| | | | DELASY | GKRKVLVKL | | | | |
| | | | RMNYP | NPRDISYVFV | | | | |
| | | | DKTHGK | FIKEAGSFIRV | | | | |
| | | | RKVLVK | PCIDPEGYTK | | | | |
| | | | LNPRDI | GLSLQEHQI | | | | |
| | | | SYVFVFI | NMKLHRDFI | | | | |
| | | | KEAGSFI | DTQMDVVS | | | | |
| | | | RVPCID | LAKARTYINS | | | | |
| | | | PEGYTK | RIQSELSEVR | | | | |
| | | | GLSLQE | QTLKKRNTK | | | | |
| | | | HQINM | GINKIARYRD | | | | |
| | | | KLHRDF | IGSQTTTGLL | | | | |
| | | | IDTQM | SGPQLSESK | | | | |
| | | | DVVSLA | DDVPIQPKT | | | | |
| | | | KARTYI | TPPQLEDD | | | | |
| | | | NSRIQS | WDSFTSGLE | | | | |
| | | | ELSEVR | PY* (SEQ ID | | | | |
| | | | QTLKKR | NO: 199) | | | | |
| | | | NTKGIN | | | | | |
| | | | KIARYR | | | | | |
| | | | DIGSQT | | | | | |
| | | | TTGLLS | | | | | |
| | | | GPQLSE | | | | | |
| | | | SKDDVP | | | | | |
| | | | IQPKTT | | | | | |
| | | | PPQLED | | | | | |
| | | | DWDSF | | | | | |
| | | | TSGLEP | | | | | |
| | | | Y (SEQ | | | | | |
| | | | ID | | | | | |
| | | | NO: 198) | | | | | |
| 33 | Kleb- | MYRRH | NSLFICS | MYRRHLHHS | MKLSSLKEEK | MKLSSLKEE | MHFLIRPEP | MPKKKRKV |
| | siella | LHHSRV | FPFEDE | RVKNLFKFAS | LISFINCFVET | KLISFINCFV | VCDESLESYL | GSGEQKLIS |
| | oxytoca | KNLFKF | FTLSQE | VRMGIVLTL | PFLNEIEKDF | ETPFLNEIEK | LRLSQDNGF | EEDLEQKLI |
| | strain | ASVRM | NEVKM | ESSLEFDTCF | DRLRYNRFL | DFDRLRYN | EHYRILSGSL | SEEDLEQKL |
| | 67 | GIVLTLE | STDESS | QLEYSPAVKT | GGEPQCMLL | RFLGGEPQ | KERLLQSDYE | ISEEDLGSG |
| | Ga02272 | SSLEFDT | DIILPAT | YISQPEGFYY | TGDTGTGKT | CMLLTGDT | AAGAFPLEL | HFLIRPEPV |
| | 27119 | CFQLEY | LDCYSEI | EFEGKSYPYT | FLLHHYMSK | GTGKTFLLH | AKVNIFHASY | CDESLESYL |
| | | SPAVKT | LKEESV | PDFLVKDQN | YPAQNGSGY | HYMSKYPA | SSYLRIRALCL | LRLSQDNG |
| | | YISQPE | RRLNYI | DQEFLLEVKP | LRKPLLVSRIP | QNGSGYLR | IADLTGQPH | FEHYRILSG |
| | | GFYYEF | QWVEK | SSQIDDIDFL | SKPSLESTM | KPLLVSRIPS | TNLLKVTLM | SLKERLLQS |
| | | EGKSYP | RIIGGW | QRFPAKQKK | VELLKDLGQ | KPSLESTM | HSTVTFGRG | DYEAAGAF |
| | | YTPDFL | TEKNITP | AKELASPLILI | WGSNYRRN | VELLKDLGQ | HKAVSRDNT | PLELAKVNI |
| | | VKDQN | LINEVA | TEKQIRSTPL | RSSAENLTES | WGSNYRR | HIPLCFIRTNS | FHASYSSYL |
| | | DQEFLL | QTLRPP | LDNLKLVHR | LIKCMMRCE | NRSSAENLT | IPCCPECLAE | RIRALCLIAD |
| | | EVKPSS | APHWR | YAGFHSIMP | TELILIDEFQE | ESLIKCMM | HGYVRQLW | LTGQPHTN |
| | | QIDDID | QLVRW | SCNEIMELLR | LIENKTRERR | RCETELILID | HYKPYTACH | LLKVTLMH |
| | | FLQRFP | HKKYLQ | EQKEVAIFNL | NQIANRLKYI | EFQELIENK | RHRRKLLTRC | STVTFGRG |
| | | AKQKKA | HRRQIT | CESIDIPQGE | SETARIPIVLV | TRERRNQIA | PACHESLNYL | HKAVSRDN |
| | | KELASPL | ALVPNH | MYSSILLLLSR | GMPWAAKI | NRLKYISET | YSELLTHCSC | THIPLCFIRT |
| | | ILITEKQI | KNKGN | GLISGNLME | SEEPQWSSR | ARIPIVLVG | GYDLRQAFT | NSIPCCPEC |
| | | RSTPLL | KTQRVS | SEFGLVTLLK | LLIRKTIPYFK | MPWAAKIS | PPTSSDDLQL | LAEHGYVR |
| | | DNLKLV | SREEIFI | YAQQGSGPK | LTDGLSIFVR | EEPQWSSR | SSMVSDDKC | QLWHYKPY |
| | | HRYAGF | ENAILKF | KKRKVGSGY | VIKGFAARM | LLIRKTIPYF | EALSPASASQ | TACHRHRR |
| | | HSIMPS | QSKERP | PYDVPDYAY | PFRKPPEIEG | KLTDGLSIF | DKSLRYGALL | KLLTRCPAC |
| | | CNEIME | SISSMY | PYDVPDYAY | KHTILGLYSA | VRVIKGFAA | WFIMRYGES | HESLNYLYS |
| | | LLREQK | CFYCDS | PYDVPDYAG | SQGRMRTLK | RMPFRKPP | SNNEEGMLS | ELLTHCSCG |
| | | EVAIFNL | VRIFNLS | SGNSLFICSF | FLLNEAVKQ | EIEGKHTIL | AMHYFRAW | YDLRQAFT |
| | | CESIDIP | NSTERIK | PFEDEFTLSQ | ALSEDSETLT | GLYSASQG | PDNFTAELL | PPTSSDDLQ |
| | | QGEMY | TVSLNT | ENEVKMSTD | HEHIGKAFHI | RMRTLKFLL | DMMAAATI | LSSMVSDD |
| | | SSILLLLS | FYRRIKK | ESSDIILPATL | FYPEHENPFY | NEAVKQAL | KQTKSFNH | KCEALSPAS |
| | | RGLISG | LSVYQV | DCYSEILKEES | IPLENIKIYEV | SEDSETLTH | MSLTDVFGK | ASQDKSLRY |
| | | NLMESE | MNARD | VRRLNYIQW | REYSGYEIDG | EHIGKAFHI | TLSDCLYLPA | GALLWFIM |
| | | FGLVTLL | GRVAA | VEKRIIGGW | AGKEDRLIP | FYPEHENPF | RDTHRNFILH | RYGESSNN |
| | | KYAQQ | NMEFQ | TEKNITPLINE | QQLTDRIPIN | YIPLENIKIY | AFLDYLTNLV | EEGMLSA |
| | | (SEQ ID | AIDSFLP | VAQTLRPPA | QLLRK (SEQ | EVREYSGSG | MENPRSNIA | MHYFRAW |
| | | NO: 204) | TSRVLE | PHWRQLVR | ID NO: 207) | PAAKKKKL | NPGDLLLSIR | PDNFTAELL |
| | | | RVEIDH | WHKKYLQH | | DGSGGYEI | DAACLLSTSN | DMMAAAT |
| | | | TPLDLIL | RRQITALVP | | DGAGKEDR | AQVYRLLDD | KQTKSFNH |
| | | | LDDELLL | NHKNKGNK | | LIPQQLTDR | GFLKVAIRPR | MSLTDVFG |
| | | | PLGRPS | TQRVSSREEI | | IPINQLLRK* | AGMKVKIST | KTLSDCLYL |
| | | | LTLLIDV | FIENAILKFQS | | (SEQ ID | PVLHLRQVIE | PARDTHRN |
| | | | YSHCAV | KERPSISSMY | | NO: 208) | FRLTHIPGPH | FILHAFLDYL |
| | | | GFNLCF | CFYCDSVRIF | | | DKGHTYLSA | TNLVMENP |
| | | | TQPGYE | NLSNSTERIK | | | R (SEQ ID | RSNIANPG |
| | | | SVRCAL | TVSLNTFYRR | | | NO: 209) | DLLLSIRDA |
| | | | LHSLVR | IKKLSVYQV | | | | ACLLSTSNA |
| | | | KDYVQE | MNARDGRV | | | | QVYRLLDD |
| | | | QYPCIE | AANMEFQAI | | | | GFLKVAIRP |
| | | | NSWISY | DSFLPTSRVL | | | | RAGMKVKI |
| | | | GKPETL | ERVEIDHTPL | | | | STPVLHLRQ |
| | | | VVDNG | DLILLDDELLL | | | | VIEFRLTHIP |
| | | | AEFWSS | PLGRPSLTLLI | | | | GPHDKGHT |
| | | | SLEHAC | DVYSHCAVG | | | | YLSAR (SEQ |
| | | | LELGINT | FNLCFTQPG | | | | ID NO: 210) |
| | | | QYNPV | YESVRCALLH | | | | |
| | | | RKPWLK | SLVRKDYVQ | | | | |
| | | | PLIERM | EQYPCIENS | | | | |
| | | | FGTINR | WISYGKPETL | | | | |
| | | | KFLESIP | VVDNGAEF | | | | |
| | | | GKTFSN | WSSSLEHAC | | | | |
| | | | ILDKAD | LELGINTQYN | | | | |
| | | | YNPQK | PVRKPWLKP | | | | |
| | | | DAVMR | LIERMFGTIN | | | | |
| | | | FSVFLEI | RKFLESIPGK | | | | |
| | | | FHHWL | TFSNILDKAD | | | | |
| | | | LDVYHY | YNPQKDAV | | | | |
| | | | EPDSRY | MRFSVFLEIF | | | | |
| | | | RYVPAL | HHWLLDVY | | | | |
| | | | AWKYG | HYEPDSRYR | | | | |
| | | | CKVYPP | YVPALAWKY | | | | |
| | | | ATIEKN | GCKVYPPATI | | | | |
| | | | ELKKLEII | EKNELKKLEII | | | | |
| | | | LSISLRR | LSISLRRLHR | | | | |
| | | | LHRRGG | RGGIHLHHL | | | | |
| | | | IHLHHL | RYDSKELSAL | | | | |
| | | | RYDSKE | RMQYSLEEK | | | | |
| | | | LSALRM | GKKKVLVKL | | | | |
| | | | QYSLEE | NPADMSYIY | | | | |
| | | | KGKKKV | VYIDKIKSYIR | | | | |
| | | | LVKLNP | VPCVDPCKY | | | | |
| | | | ADMSYI | TQNLSLQQH | | | | |
| | | | YVYIDKI | LINLRFHRDF | | | | |
| | | | KSYIRVP | INENINLDSL | | | | |
| | | | CVDPCK | SKARIYISERI | | | | |
| | | | YTQNLS | QGEIDNVRQ | | | | |
| | | | LQQHLI | YAKRSSKKG | | | | |
| | | | NLRFHR | MKKIASHQG | | | | |
| | | | DFINENI | VTSQNKKTIA | | | | |
| | | | NLDSLS | SDTIHFPAQK | | | | |
| | | | KARIYIS | GKNRDTHTL | | | | |
| | | | ERIQGEI | PDDWDDFT | | | | |
| | | | DNVRQ | SDLEPF* | | | | |
| | | | YAKRSS | (SEQ ID | | | | |
| | | | KKGMK | NO: 206) | | | | |
| | | | KIASHQ | | | | | |
| | | | GVTSQ | | | | | |
| | | | NKKTIA | | | | | |
| | | | SDTIHFP | | | | | |
| | | | AQKGK | | | | | |
| | | | NRDTHT | | | | | |
| | | | LPDDW | | | | | |
| | | | DDFTSD | | | | | |
| | | | LEPF | | | | | |
| | | | (SEQ ID | | | | | |
| | | | NO: 205) | | | | | |
| 36 | Pseudo. | MYIRNL | MGYTM | MYIRNLRKP | MNALTEIQIE | MNALTEIQI | MAFLFSPKA | MPKKKRKV |
| | arctica | RKPSPN | TDFFDE | SPNKNVFKF | QLRNFSDCIV | EQLRNFSD | RAFSDESLES | GSGEQKLIS |
| | A 37-1- | KNVFKF | FNESLA | ASTKVGNVI | MHPQIKAIF | CIVMHPQI | YLLRVVSENF | EEDLEQKLI |
| | 2 | ASTKVG | PLKPQT | MCESTLEFN | NDFDELRLN | KAIFNDFDE | FDSYEGLSLA | SEEDLEQKL |
| | chromo- | NVIMCE | PTRYLKL | ACFHNEYND | RKFQSDQQ | LRLNRKFQS | IREELHELDF | ISEEDLGSG |
| | some 1 | STLEFN | DDANLI | LIESYGSQPE | GMLLIGDTG | DQQGMLLI | EAHGAFPIDL | AFLFSPKAR |
| | | ACFHNE | KRDLDT | GFKYEFMGK | VGKSHTINH | GDTGVGKS | KRLNVYHAK | AFSDESLES |
| | | YNDLIES | FSNTLK | SLPYTPDTVV | YKKRVLATQ | HTINHYKKR | HNSHFRMR | YLLRVVSEN |
| | | YGSQPE | NEALQR | VYKDKCVKY | NYSRNTMPV | VLATQNYS | ALGLLETLLD | FFDSYEGLS |
| | | GFKYEF | YKLIISID | HEYKYETETA | LISRISRGKGL | RNTMPVLIS | LPRYELQKLA | LAIREELHEL |
| | | MGKSLP | KKLSAG | EPLFRERFSA | DATLIQMLA | RISRGKGLD | LLKSDIKFNS | DFEAHGAF |
| | | YTPDTV | WTQRN | KRAACLKMG | DLELFGSSQ | ATLIQMLA | SAALYKNGV | PIDLKRLNV |
| | | VVYKDK | LDPILDE | VQLILVTENQ | MKKRGYKTE | DLELFGSSQ | DIPQKFIRYH | YHAKHNSH |
| | | CVKYHE | IFKEDE | ITKGLALNNF | LTKKLVESLIK | MKKRGYKT | TEAAVDSIPV | FRMRALGL |
| | | YKYETE | QARPN | KLLHRYSGVY | AQVELLIINE | ELTKKLVES | CPQCLAEEA | LETLLDLPR |
| | | TAEPLF | WRTVA | GIKNIQSEML | FQELIEFKSV | LIKAQVELLI | YIKQSWHIK | YELQKLALL |
| | | RERFSA | RWRKK | NFINKSGAIN | QERQQIANG | INEFQELIEF | WVDACTKH | KSDIKFNSS |
| | | KRAACL | YIESNG | LVDVKSQFN | LKFISEEAKV | KSVQERQQ | QCTLAHNCP | AALYKNGV |
| | | KMGVQ | DLASLV | LSIGEARSFLY | PIVLVGMPW | IANGLKFISE | ECCAPINYIE | DIPQKFIRY |
| | | LILVTEN | VKNHK | ALLHKGLLKA | AAKIAEEPQ | EAKVPIVLV | NESITHCSCG | HTEAAVDSI |
| | | QITKGL | MGNRN | DLEDDDLSN | WASRLVRKR | GMPWAAK | FELTWASTS | PVCPQCLA |
| | | ALNNFK | KRIEGD | NPTLWVTPG | KLEYFSLKND | IAEEPQWA | PVNALSIEHL | EEAYIKQS |
| | | LLHRYS | ESFFDK | SGPKKKRKV | SKYFRQYLM | SRLVRKRKL | NKLLDKSER | WHIKWVD |
| | | GVYGIK | ALERFL | GSGYPYDVP | GLVKQMPF | EYFSLKNDS | NDSHSLFNN | ACTKHQCT |
| | | NIQSEM | DAKRPT | DYAYPYDVP | DEPPKLESKH | KYFRQYLM | TTLTERFAAL | LAHNCPEC |
| | | LNFINKS | IATAYQ | DYAYPYDVP | TTMALFAAC | GLVKQMPF | LWYQGRYS | CAPINYIEN |
| | | GAINLV | YYKDLIV | DYAGSGTDF | RGENRALKH | DEPPKLESK | QTDNFCLDD | ESITHCSCG |
| | | DVKSQF | IENESIV | FDEFNESLAP | LLMEALKLAL | HTTMALFA | AVDYFSMW | FELTWASTS |
| | | NLSIGE | EGKIPIIS | LKPQTPTRYL | SCNEYLENK | ACRGENRA | PAVFYKELDE | PVNALSIEH |
| | | ARSFLY | YTAFNK | KLDDANLIKR | HFIAVYEKFD | LKHLLMEAL | LSKNAEMKLI | LNKLLDKSE |
| | | ALLHKG | RIKAIPP | DLDTFSNTLK | FFNDKDSLKL | KLALSCNEY | DLFNKTEFKF | RNDSHSLF |
| | | LLKADL | YAVAVA | NEALQRYKLI | KNPFKQDIK | LENKHFIAV | IFGDAILACP | NNTTLTERF |
| | | EDDDLS | RHGKFK | ISIDKKLSAG | DIIIYEVTKNS | YEKFDFFND | STQMQREL | AALLWYQG |
| | | NNPTL | ADQWF | WTQRNLDPI | SYNPNALDP | KDSLKLKNP | HFIYRALLDY | RYSQTDNF |
| | | WVTP | AYCAAH | LDEIFKEDEQ | EDMLTGRKF | FKQDIKDIII | LVTLVEGNP | CLDDAVDY |
| | | (SEQ ID | VPPTRIL | ARPNWRTV | AIVK (SEQ ID | YEVTKNSGS | KAKKPNTAD | FSMWPAV |
| | | NO: 211) | ERVEID | ARWRKKYIE | NO: 214) | GPAAKKKK | LLVSVLEAAT | FYKELDELS |
| | | | HTPLDLI | SNGDLASLV | | LDGSGSYN | LLGTSVEQVY | KNAEMKLI |
| | | | LLDDELL | VKNHKMGN | | PNALDPED | RLYQDGILQT | DLFNKTEFK |
| | | | IPIGRPY | RNKRIEGDES | | MLTGRKFAI | AFRHKMNQ | FIFGDAILA |
| | | | LTLLIDV | FFDKALERFL | | VK* (SEQ | RINPYKGVFF | CPSTQMQR |
| | | | FSGCVL | DAKRPTIATA | | ID NO: 215) | LRHAIEYKTS | ELHFIYRALL |
| | | | GFHLSY | YQYYKDLIVI | | | FGNDKARM | DYLVTLVEG |
| | | | KSPSYV | ENESIVEGKI | | | YLSAW (SEQ | NPKAKKPN |
| | | | SAAKAI | PIISYTAFNKR | | | ID NO: 216) | TADLLVSVL |
| | | | AHAIKP | IKAIPPYAVA | | | | EAATLLGTS |
| | | | KSLDAL | VARHGKFKA | | | | VEQVYRLY |
| | | | NIQLQN | DQWFAYCA | | | | QDGILQTA |
| | | | DWPCF | AHVPPTRILE | | | | FRHKMNQ |
| | | | GKFENL | RVEIDHTPLD | | | | RINPYKGVF |
| | | | VVDNG | LILLDDELLIPI | | | | FLRHAIEYK |
| | | | AEFWSK | GRPYLTLLID | | | | TSFGNDKA |
| | | | NLEHAC | VFSGCVLGF | | | | RMYLSAW |
| | | | QSAGIN | HLSYKSPSYV | | | | (SEQ ID |
| | | | IQYNPV | SAAKAIAHAI | | | | NO: 217) |
| | | | RKPWLK | KPKSLDALNI | | | | |
| | | | PFIERFF | QLQNDWPC | | | | |
| | | | GVMNQ | FGKFENLVV | | | | |
| | | | YFLPEV | DNGAEFWS | | | | |
| | | | PGKTFS | KNLEHACQS | | | | |
| | | | NILEKEE | AGINIQYNP | | | | |
| | | | YKPEKD | VRKPWLKPFI | | | | |
| | | | AIMRFS | ERFFGVMN | | | | |
| | | | TFVEEF | QYFLPEVPG | | | | |
| | | | HRWIV | KTFSNILEKE | | | | |
| | | | DVYHQ | EYKPEKDAI | | | | |
| | | | DSNSRE | MRFSTFVEE | | | | |
| | | | TRIPIKR | FHRWIVDVY | | | | |
| | | | WQQGF | HQDSNSRET | | | | |
| | | | DVYPPL | RIPIKRWQQ | | | | |
| | | | TMNEE | GFDVYPPLT | | | | |
| | | | DEARFT | MNEEDEARF | | | | |
| | | | MLMRIS | TMLMRISDS | | | | |
| | | | DSRTLT | RTLTRNGIKY | | | | |
| | | | RNGIKY | QELMYDSTA | | | | |
| | | | QELMY | LADYRKHYP | | | | |
| | | | DSTALA | QTKETLKKLI | | | | |
| | | | DYRKHY | KVDPDDISKI | | | | |
| | | | PQTKET | YVYLEELESYL | | | | |
| | | | LKKLIKV | EVPCTDPTG | | | | |
| | | | DPDDIS | YTDGLSIYEH | | | | |
| | | | KIYVYLE | KTIKKVNRET | | | | |
| | | | ELESYLE | IRESKNSLGL | | | | |
| | | | VPCTDP | AKARMAIHE | | | | |
| | | | TGYTDG | RVKQEQEVF | | | | |
| | | | LSIYEHK | IASKTKAKIT | | | | |
| | | | TIKKVN | AVKKQAQIA | | | | |
| | | | RETIRES | DVSNTGKGT | | | | |
| | | | KNSLGL | IKVSEESAAP | | | | |
| | | | AKARM | VHKNISNDA | | | | |
| | | | AIHERV | FDDWDDDL | | | | |
| | | | KQEQEV | EAFE* (SEQ | | | | |
| | | | FIASKTK | ID NO: 213) | | | | |
| | | | AKITAV | | | | | |
| | | | KKQAQI | | | | | |
| | | | ADVSNT | | | | | |
| | | | GKGTIK | | | | | |
| | | | VSEESA | | | | | |
| | | | APVHK | | | | | |
| | | | NISNDA | | | | | |
| | | | FDDWD | | | | | |
| | | | DDLEAF | | | | | |
| | | | E (SEQ | | | | | |
| | | | ID | | | | | |
| | | | NO: 212) | | | | | |
| 37 | Pseud. | MYRRKL | MFNND | MYRRKLKYS | MLTDKQKEK | MLTDKQKE | MHFLVQTKS | MPKKKRKV |
| | trans- | KYSRVK | LFDDEF | RVKNLHKFA | LNEFRDVFIE | KLNEFRDVF | YPDEALESYL | GSGEQKLIS |
| | lucida | NLHKFA | NQPLPK | SQKNKSTCL | YPIITTIFNDF | IEYPIITTIFN | LRLARDNSY | EEDLEQKLI |
| | KMM 520 | SQKNKS | AETKLP | VESSLEFDAC | DRLRLGKGL | DFDRLRLGK | NGYSELADIL | SEEDLEQKL |
| | | TCLVESS | QNYTK | FHFEFSPPIA | TGEKPCMLL | GLTGEKPC | WQWLAEQ | ISEEDLGSG |
| | | LEFDAC | DLQALP | AFEAQPLGY | NGDTGTGKT | MLLNGDTG | DNELEGALP | HFLVQTKSY |
| | | FHFEFS | EKIKTTT | EYEFDNRICR | ALIKQYKERH | TGKTALIKQ | LALSKVDVY | PDEALESYL |
| | | PPIAAFE | FAKLKYI | YTPDFLLTHT | LPQFINGVM | YKERHLPQF | HARQASSFRI | LRLARDNSY |
| | | AQPLGY | QWLEA | DGTQKFIEVK | NHPVLVSRIP | INGVMNHP | RALKLVAQL | NGYSELADI |
| | | EYEFDN | NIQGG | PQSKIADEDF | SNPTLESTLA | VLVSRIPSN | ADVNAGDIL | LWQWLAE |
| | | RICRYTP | WTQKN | RARFIEKQAI | ELLKDLGQV | PTLESTLAEL | ALAWRRSNF | QDNELEGA |
| | | DFLLTH | LEPLLKL | AKQDGRDLI | GSTERKLRIN | LKDLGQVG | KFGNLAAVS | LPLALSKVD |
| | | TDGTQK | MPDVE | LVTDKQIRVY | GTRLTTSLIK | STERKLRIN | RNELAIPLELL | VYHARQAS |
| | | FIEVKP | GEKKPS | PTLNNLKLLH | CLKTCGTELII | GTRLTTSLIK | RTDNIPVCIK | SFRIRALKLV |
| | | QSKIAD | WRTAA | RYSGFQSLTE | IDEFQELIEH | CLKTCGTEL | CLSESSHIPFY | AQLADVNA |
| | | EDFRAR | RWYSA | LQASVLELVK | NQGKKRREI | IIIDEFQELIE | WHLKPYKAC | GDILALAW |
| | | FIEKQAI | YTNADK | QYGSIKVGQ | ANRLKYINDE | HNQGKKRR | HKHKSQLITR | RRSNFKFG |
| | | AKQDG | NIMALI | LIRYLKVTAG | AGVSIVLVG | EIANRLKYI | CKECYDLIDY | NLAAVSRN |
| | | RDLILVT | PSHQKK | ELLATVLRLL | MPWAEKIA | NDEAGVSI | RASEAFLECV | ELAIPLELLR |
| | | DKQIRV | GNRER | SLGQLFADLT | DEPQWSSRL | VLVGMPW | CGCKITNSE | TDNIPVCIK |
| | | YPTLNN | DTTTDK | TNEISIETAI | LIRRQLPYFK | AEKIADEPQ | QLNDADFKI | CLSESSHIPF |
| | | LKLLHR | FFEKALE | WSNNVGSG | LSENPKHFV | WSSRLLIRR | AIALASSNSQ | YWHLKPYK |
| | | YSGFQS | RYLVKE | PKKKRKVGS | QLIIGLANR | QLPYFKLSE | KIVGLISWFA | ACHKHKSQ |
| | | LTELQA | KPSVAS | GYPYDVPDY | MPFAEKPNL | NPKHFVQLI | KVKQLDVSD | LITRCKECY |
| | | SVLELV | AYKFYK | AYPYDVPDY | SEQATVFTLF | IGLANRMP | ADFNCAFVD | DLIDYRASE |
| | | KQYGSI | DLVIIEN | AYPYDVPDY | SLSKGCFRTL | FAEKPNLSE | YFNTWPESL | AFLECVCGC |
| | | KVGQLI | DSVVDS | AGSGFNNDL | KYFLDDAVLY | QATVFTLFS | TTELDLLTNN | KITNSEQLN |
| | | RYLKVT | VLKPLTY | FDDEFNQPL | ALMDNAKTL | LSKGCFRTL | ARLKQLNPF | DADFKIAIA |
| | | AGELLA | KAFKNR | PKAETKLPQ | TTKHLVKAFE | KYFLDDAVL | NKTKFSSVY | LASSNSQKI |
| | | TVLRLLS | IDNLPQ | NYTKDLQAL | VLFPDVPNLF | YALMDNAK | GDLIRDGQIA | VGLISWFA |
| | | LGQLFA | YEVMIA | PEKIKTTTFA | TLPVAEITAS | TLTTKHLVK | ATSNRKNKVI | KVKQLDVS |
| | | DLTTNEI | RYGKRL | KLKYIQWLE | EVERYSLYKP | AFEVLFPDV | DEIISYFVELV | DADFNCAF |
| | | SIETAIW | ADIAYN | ANIQGGWT | ESSQDEDPFI | PNLFTLPVA | DSNPKAKHP | VDYFNTWP |
| | | SNNV | KVEGHK | QKNLEPLLKL | ATKFTDRMP | EITASEVER | NIGDLLLCTF | ESLTTELDLL |
| | | (SEQ ID | RPIRVLE | MPDVEGEKK | ISQLLRK | YSGSGPAA | DAAVLLNTT | TNNARLKQ |
| | | NO: 218) | KVEIDH | PSWRTAAR | (SEQ ID | KKKKLDGS | TEQVYRLHQ | LNPFNKTKF |
| | | | TPLDLIL | WYSAYTNA | NO: 221) | GLYKPESSQ | EAFLNCAYS | SSVYGDLIR |
| | | | LDDELH | DKNIMALIPS | | DEDPFIATK | QKKHEQLRA | DGQIAATS |
| | | | IPLGRPT | HQKKGNRER | | FTDRMPIS | DSHVFYLRQ | NRKNKVID |
| | | | LTMLVD | DTTTDKFFEK | | QLLRK* | VIELQQAFA | EIISYFVELV |
| | | | VYSHCI | ALERYLVKEK | | (SEQ ID | AEKPLTKKQF | DSNPKAKH |
| | | | VGYYFS | PSVASAYKFY | | NO: 222) | IAPW (SEQ | PNIGDLLLC |
| | | | FSEPSY | KDLVIIENDS | | | ID NO: 223) | TFDAAVLL |
| | | | DAVRR | VVDSVLKPLT | | | | NTTTEQVY |
| | | | AMLNA | YKAFKNRID | | | | RLHQEAFL |
| | | | MKPKSE | NLPQYEVMI | | | | NCAYSQKK |
| | | | VAKLYP | ARYGKRLADI | | | | HEQLRADS |
| | | | DTINEW | AYNKVEGHK | | | | HVFYLRQVI |
| | | | KCAGKI | RPIRVLEKVEI | | | | ELQQAFAA |
| | | | ETLVVD | DHTPLDLILL | | | | EKPLTKKQF |
| | | | NGAEF | DDELHIPLGR | | | | IAPW (SEQ |
| | | | WSNSLE | PTLTMLVDV | | | | ID NO: 224) |
| | | | LACEEIG | YSHCIVGYYF | | | | |
| | | | INTQYN | SFSEPSYDAV | | | | |
| | | | PVAKP | RRAMLNAM | | | | |
| | | | WLKPFV | KPKSEVAKLY | | | | |
| | | | ERMFG | PDTINEWKC | | | | |
| | | | TINTELL | AGKIETLVVD | | | | |
| | | | DPVPGK | NGAEFWSN | | | | |
| | | | TFSNILQ | SLELACEEIGI | | | | |
| | | | KHEYNP | NTQYNPVAK | | | | |
| | | | KKDAIM | PWLKPFVER | | | | |
| | | | RFTTFM | MFGTINTELL | | | | |
| | | | QLFHK | DPVPGKTFS | | | | |
| | | | WVVDV | NILQKHEYN | | | | |
| | | | YHQDA | PKKDAIMRF | | | | |
| | | | DSRFKYI | TTFMQLFHK | | | | |
| | | | PSQLW | WVVDVYHQ | | | | |
| | | | DQGFN | DADSRFKYIP | | | | |
| | | | TLPPTM | SQLWDQGF | | | | |
| | | | LSDADL | NTLPPTMLS | | | | |
| | | | QQLDV | DADLQQLDV | | | | |
| | | | VLSISN | VLSISNHRVL | | | | |
| | | | HRVLRK | RKGGIRLENL | | | | |
| | | | GGIRLE | SYDSTELANY | | | | |
| | | | NLSYDS | RKQFSHKVS | | | | |
| | | | TELANY | QEVLIKLNPD | | | | |
| | | | RKQFSH | DISYIYVYLDK | | | | |
| | | | KVSQEV | LEHYIKVPCI | | | | |
| | | | LIKLNPD | DPNGYTQNL | | | | |
| | | | DISYIYV | SLNQHKINIR | | | | |
| | | | YLDKLE | IHRDFISGSID | | | | |
| | | | HYIKVP | NVGLAKAR | | | | |
| | | | CIDPNG | MFIHNKIQN | | | | |
| | | | YTQNLS | EFEELKNAPK | | | | |
| | | | LNQHKI | HSKVKGGKA | | | | |
| | | | NIRIHR | LAKHQNISS | | | | |
| | | | DFISGSI | DSQKSITHSK | | | | |
| | | | DNVGL | PVEAKKVTP | | | | |
| | | | AKARM | KEQPTDSW | | | | |
| | | | FIHNKI | DDFISDLDGF | | | | |
| | | | QNEFEE | * (SEQ ID | | | | |
| | | | LKNAPK | NO: 220) | | | | |
| | | | HSKVKG | | | | | |
| | | | GKALAK | | | | | |
| | | | HQNISS | | | | | |
| | | | DSQKSI | | | | | |
| | | | THSKPV | | | | | |
| | | | EAKKVT | | | | | |
| | | | PKEQPT | | | | | |
| | | | DSWDD | | | | | |
| | | | FISDLD | | | | | |
| | | | GF (SEQ | | | | | |
| | | | ID | | | | | |
| | | | NO: 219) | | | | | |
| 38 | Shewan- | MYIRNL | MDFAD | MYIRNLRKP | MTKLTLQQD | MTKLTLQQ | MAFLFSPKSL | MPKKKRKV |
| | ella_ | RKPSPN | EFTESTS | SPNKNVFKF | TALKEFGLCF | DTALKEFGL | AFSGESLESY | GSGEQKLIS |
| | piezo- | KNVFKF | AKKPET | ASAKVSETI | IELPIVSETFQ | CFIELPIVSE | LLRVVAENFF | EEDLEQKLI |
| | tol- | ASAKVS | PAQYVK | MCESTLEFD | DFDDLRFNR | TFQDFDDL | DSYQQLSLAI | SEEDLEQKL |
| | erans_ | ETIMCE | LDDAEL | ACFHHEYNE | DYQSDPQC | RFNRDYQS | REELHELDFE | ISEEDLGSG |
| | WP3 | STLEFD | LKRDLD | TIETFGSQPK | MMLTGETG | DPQCMML | AHGAFPIELK | AFLFSPKSL |
| | uid58745 | ACFHHE | TFPDFL | GFYYRFEGK | SGKTRLIQEY | TGETGSGK | RLNVYHAKH | AFSGESLES |
| | | YNETIET | KEKALD | RLPYTPDAIL | RRRVNANSG | TRLIQEYRR | NSHFRMRAL | YLLRVVAEN |
| | | FGSQPK | KYKLISFI | HYIDGTTKFH | FRHSDVPVLI | RVNANSGF | SLLESLLDLPP | FFDSYQQLS |
| | | GFYYRF | EQENSG | EYKPYSKTFD | TNISSNKGLE | RHSDVPVLI | HELQKLALLR | LAIREELHEL |
| | | EGKRLP | GWTQK | PIFRAKFVAK | NTLVQILSDL | TNISSNKGL | SNRRFVGG | DFEAHGAF |
| | | YTPDAIL | KLDPILD | KEAAQALGT | DTFGCHQKK | ENTLVQILS | MSAVHRNGI | PIELKRLNV |
| | | HYIDGT | RLFEGN | ELILVTDKQI | RGMKTDLTK | DLDTFGCH | DIPLSFIRCA | YHAKHNSH |
| | | TKFHEY | TEKRPN | RVNPILNNLK | KVVRNLIAA | QKKRGMKT | DKDGIESVPI | FRMRALSLL |
| | | KPYSKT | WRTVV | LLHRYSGIYG | NVELLIINEF | DLTKKVVR | CPQCLKEGP | ESLLDLPPH |
| | | FDPIFR | RWRKS | VTDIQRELLQ | HDLIKFKNYQ | NLIAANVEL | YIRQAWHIK | ELQKLALLR |
| | | AKFVAK | YIDSNG | LVRKSDNIQL | EIQIITSALKFI | LIINEFHDLI | PIEVCAKHG | SNRRFVGG |
| | | KEAAQA | DLASLV | ADVASEYNL | SEAANIPIVL | KFKNYQEIQ | CELINHCPDC | MSAVHRN |
| | | LGTELIL | VKRHK | PIAETRSFLYS | VGMPWMK | IITSALKFISE | QQPINYIENE | GIDIPLSFIR |
| | | VTDKQI | MGNRK | LINKGLIKAD | DIINDSEWG | AANIPIVLV | SITHCACGFD | CADKDGIES |
| | | RVNPIL | KRVEGD | LNQDDLSCN | SRLRRRKHLE | GMPWMK | FTTASSVKAD | VPICPQCLK |
| | | NNLKLL | EVFFER | PSVWCHAG | YFSYIRKEDR | DIINDSEW | SQAVLLSRSL | EGPYIRQA |
| | | HRYSGI | ALSRFL | SGPKKKRKV | EHFRLLLVGF | GSRLRRRK | FDGDALSNN | WHIKPIEVC |
| | | YGVTDI | DAKRPK | GSGYPYDVP | SKRMSFDTR | HLEYFSYIRK | PLLFMGTSV | AKHGCELIN |
| | | QRELLQ | VTTAYQ | DYAYPYDVP | PVLHSKELTR | EDREHFRLL | THRFAALIW | HCPDCQQP |
| | | LVRKSD | YYKDVIT | DYAYPYDVP | ALFAVCRGE | LVGFSKRM | YQKCHARNT | INYIENESIT |
| | | NIQLAD | IENETIV | DYAGSGDFA | FRQLMVFLY | SFDTRPVLH | ECMAHRAV | HCACGFDF |
| | | VASEYN | DGKIPII | DEFTESTSAK | EACKMALQ | SKELTRALF | GYFEDWPTS | TTASSVKAD |
| | | LPIAETR | SYTAFN | KPETPAQYV | NNDHTLNEK | AVCRGEFR | FYRELDAVTT | SQAVLLSRS |
| | | SFLYSLI | QRIKSLP | KLDDAELLKR | TLAETFDKLG | QLMVFLYE | GAEARLIDLF | LFDGDALS |
| | | NKGLIK | PYPIAV | DLDTFPDFLK | CEHLSSNPFT | ACKMALQ | NRTSFRSIYG | NNPLLFMG |
| | | ADLNQ | ARHGKF | EKALDKYKLI | IKFKEIPIPVL | NNDHTLNE | ELILDSQCLLP | TSVTHRFA |
| | | DDLSCN | KADQW | SFIEQENSGG | SIPSRYNPNA | KTLAETFDK | EDKDPHFIYL | ALIWYQKC |
| | | PSVWC | FAYCSS | WTQKKLDPI | LEEKDEIIDR | LGCEHLSSN | ALMEYISKLV | HARNTECM |
| | | HA (SEQ | HIPPTRI | LDRLFEGNTE | VFEYIY (SEQ | PFTIKFKEIPI | ESHPKSKKP | AHRAVGYF |
| | | ID | LERVEID | KRPNWRTV | ID NO: 228) | PVLSIPSGS | NVADMLVT | EDWPTSFY |
| | | NO: 225) | HTPLDLI | VRWRKSYID | | GPAAKKKK | VAEIAVLLST | RELDAVTT |
| | | | LLDDELL | SNGDLASLV | | LDGSGRYN | THEQVYRLY | GAEARLIDL |
| | | | IPLGRPY | VKRHKMGN | | PNALEEKDE | QDGVLTAG | FNRTSFRSI |
| | | | LTLIVDV | RKKRVEGDE | | IIDRVFEYIY | MRSKIRTRIS | YGELILDSQ |
| | | | FSNCVL | VFFERALSRF | | (SEQ ID | PHIGVFYLRQ | CLLPEDKDP |
| | | | GFHLSY | LDAKRPKVT | | NO: 229) | VIEYKTSFGN | HFIYLALME |
| | | | KAPSYV | TAYQYYKDVI | | | DKQGMYLS | YISKLVESH |
| | | | SAAKA | TIENETIVDG | | | AW (SEQ ID | PKSKKPNV |
| | | | VHAIKP | KIPIISYTAFN | | | NO: 230) | ADMLVTVA |
| | | | KTLSNIG | QRIKSLPPYPI | | | | EIAVLLSTT |
| | | | IELQND | AVARHGKFK | | | | HEQVYRLY |
| | | | WPCYG | ADQWFAYC | | | | QDGVLTAG |
| | | | KFETLV | SSHIPPTRILE | | | | MRSKIRTRI |
| | | | VDNGA | RVEIDHTPLD | | | | SPHIGVFYL |
| | | | EFWSKS | LILLDDELLIP | | | | RQVIEYKTS |
| | | | LDHACK | LGRPYLTLIV | | | | FGNDKQG |
| | | | EAGINI | DVFSNCVLG | | | | MYLSAW |
| | | | QYNPV | FHLSYKAPSY | | | | (SEQ ID |
| | | | RKPWLK | VSAAKAIVH | | | | NO: 231) |
| | | | PFVERF | AIKPKTLSNI | | | | |
| | | | FGMIN | GIELQNDWP | | | | |
| | | | QYFLTEI | CYGKFETLVV | | | | |
| | | | PGKTFS | DNGAEFWS | | | | |
| | | | NILEKE | KSLDHACKE | | | | |
| | | | DYKPEK | AGINIQYNP | | | | |
| | | | DAIMRF | VRKPWLKPF | | | | |
| | | | SVFVEE | VERFFGMIN | | | | |
| | | | FHRWIV | QYFLTEIPGK | | | | |
| | | | DIYHQD | TFSNILEKED | | | | |
| | | | SDSRDT | YKPEKDAIM | | | | |
| | | | RIPIKQ | RFSVFVEEFH | | | | |
| | | | WQHGF | RWIVDIYHQ | | | | |
| | | | DIYPPL | DSDSRDTRIP | | | | |
| | | | QMEVE | IKQWQHGF | | | | |
| | | | DEKRFN | DIYPPLQME | | | | |
| | | | VLMGIA | VEDEKRFNV | | | | |
| | | | DERTLT | LMGIADERT | | | | |
| | | | RNGFKF | LTRNGFKFEE | | | | |
| | | | EELMYD | LMYDSTALA | | | | |
| | | | STALAD | DYRKHYPQT | | | | |
| | | | YRKHYP | KDTIKKLIKID | | | | |
| | | | QTKDTI | PDDLSSIHVY | | | | |
| | | | KKLIKID | LEELEGYLKV | | | | |
| | | | PDDLSSI | PCTDTTGYT | | | | |
| | | | HVYLEE | QGLSLHEHK | | | | |
| | | | LEGYLK | VTKKINREIIR | | | | |
| | | | VPCTDT | ESKDNLGLA | | | | |
| | | | TGYTQG | KARMAIHAR | | | | |
| | | | LSLHEH | VQQEQELFN | | | | |
| | | | KVTKKI | ESKTKTKLSG | | | | |
| | | | NREIIRE | VKKKAQLAD | | | | |
| | | | SKDNLG | ISSTGKSTIVL | | | | |
| | | | LAKAR | PESEPQKSIN | | | | |
| | | | MAIHA | CNQVEAEM | | | | |
| | | | RVQQE | EDDDWDM | | | | |
| | | | QELFNE | DLEGY* | | | | |
| | | | SKTKTKL | (SEQ ID | | | | |
| | | | SGVKKK | NO: 227) | | | | |
| | | | AQLADI | | | | | |
| | | | SSTGKS | | | | | |
| | | | TIVLPES | | | | | |
| | | | EPQKSI | | | | | |
| | | | NCNQV | | | | | |
| | | | EAEME | | | | | |
| | | | DDDWD | | | | | |
| | | | MDLEG | | | | | |
| | | | Y (SEQ | | | | | |
| | | | ID | | | | | |
| | | | NO: 226) | | | | | |
| 40 | V. | MYIRNL | MSRRIK | MYIRNLRKP | MPNSALNYP | MPNSALNY | MDQHEAIA | MPKKKRKV |
| | azureus | RKPSPN | DEFDPA | SPNKNIFKFA | IDLILSDYHDS | PIDLILSDYH | GAFPLELNR | GSGEQKLIS |
| | strain | KNIFKF | YSEAIE | SAKNQGSIM | FTIYPEVEKV | DSFTIYPEV | VNIYHAQTT | EEDLEQKLI |
| | LC2-005 | ASAKN | QEFLSH | CEGSLERDC | FAGLDWLVR | EKVFAGLD | SQMRVRVLI | SEEDLEQKL |
| | | QGSIMC | PETIRT | CYHFEYDPN | RRNFGSFVP | WLVRRRNF | HLENQFKLN | ISEEDLGSG |
| | | EGSLER | QLQYNS | VVSFESQPR | SMLLTGGTG | GSFVPSML | NFGVLRLALS | DQHEAIAG |
| | | DCCYHF | LAKTQT | GFFYDFDGK | SGKSASIKHY | LTGGTGSG | HSKAQFSPQ | AFPLELNRV |
| | | EYDPNV | YERDLA | QLPYTPDFFV | IDNNLSDSEV | KSASIKHYID | YKAVHRFGV | NIYHAQTTS |
| | | VSFESQ | SFPPEQ | VYDDGCHSF | LLTRVRPTLH | NNLSDSEVL | DYPYAFLRKR | QMRVRVLI |
| | | PRGFFY | KEKALE | MEIKPYSKTL | ETLLWMAK | LTRVRPTLH | FTPICPLCIDE | HLENQFKL |
| | | DFDGK | RYKLLCL | SKEFKLKFQS | NLNAYRNSR | ETLLWMAK | APYIRQQW | NNFGVLRL |
| | | QLPYTP | IENELR | RKRAAELLGF | AKPSDIGLM | NLNAYRNS | QFISDQVCQ | ALSHSKAQ |
| | | DFFVVY | GGWTP | NLILVTDRQI | DRVIGCLKKA | RAKPSDIGL | YHGCKLIHRC | FSPQYKAV |
| | | DDGCH | RNLDPLI | RAGYFLKNS | NLKLLIIEECQ | MDRVIGCL | PECKSRLEYQ | HRFGVDYP |
| | | SFMEIK | DKYSSN | QMVHRYSG | ELFECTSHKE | KKANLKLLII | SAESINQCEC | YAFLRKRFT |
| | | PYSKTLS | VSIPKPS | CIADDSLIDIV | RQDIRDRLK | EECQELFEC | GYELRNSPIE | PICPLCIDEA |
| | | KEFKLKF | YKTLIR | FAELLLSEVV | MISDDCKLPI | TSHKERQDI | DAPEAELLV | PYIRQQWQ |
| | | QSRKRA | WQKNF | KISVLARRIS | VFVGIPSAKL | RDRLKMIS | AQWLSGNN | FISDQVCQY |
| | | AELLGF | TKSDGN | GFTLGEVFAS | ILEDSQWQR | DDCKLPIVF | SKPLWLLKA | HGCKLIHRC |
| | | NLILVTD | LISLVDK | VLRLIAVGRA | RIMVKRELPY | VGIPSAKLIL | EMTISERYGF | PECKSRLEY |
| | | RQIRAG | NYLKGN | KIDLDLELLN | VKITDDSSID | EDSQWQR | LLWYVNRYG | QSAESINQC |
| | | YFLKNS | RVARKT | ENSTVSVYG | RYLDLLEAM | RIMVKRELP | EFDELSFESFI | ECGYELRNS |
| | | QMVHR | GDEAFY | SGPKKKRKV | QASVPIPFEV | YVKITDDSSI | EYCSDWPTV | PIEDAPEAE |
| | | YSGCIA | ERALER | GSGYPYDVP | DLTDVDSAV | DRYLDLLEA | LWQELDGLK | LLVAQWLS |
| | | DDSLIDI | FLDSVR | DYAYPYDVP | RLLAASRGIL | MQASVPIP | EKAEVVRVK | GNNSKPLW |
| | | VFAELLL | PSISAAY | DYAYPYDVP | SNMKELIAS | FEVDLTDV | NWKKMFFN | LLKAEMTIS |
| | | SEVVKIS | QFYCDE | DYAGSGSRRI | AIESSLHLGR | DSAVRLLAA | EAFGSLLKDC | ERYGFLLW |
| | | VLARRIS | ITIANEQ | KDEFDPAYS | QTIRLDDFRL | SRGILSNM | RQLPSRQLN | YVNRYGEF |
| | | GFTLGE | VISGQV | EAIEQEFLSH | GYEAIYGVD | KELIASAIES | HNIVLKQVL | DELSFESFIE |
| | | VFASVL | PIVSYQ | PETIRTQLQY | EANPFSINA | SLHLGRQTI | AYFTRLIATV | YCSDWPTV |
| | | RLIAVG | TFKKRIK | NSLAKTQTY | DELVIKQIES | RLDDFRLGY | PSSAKGNIG | LWQELDGL |
| | | RAKIDL | KEQPYN | ERDLASFPPE | YEEYVVDAA | EAIYGVDEA | DLLLSPLEAS | KEKAEVVR |
| | | DLELLN | IVLARH | QKEKALERY | NGELKFVQQ | NPFSINADE | TLLSCTTDEV | VKNWKKM |
| | | ENSTVS | GKYYAD | KLLCLIENELR | IFNELTIEQLL | LVIKQIESYE | YRLYEFGEIK | FFNEAFGSL |
| | | VY (SEQ | KLYHYY | GGWTPRNL | G (SEQ ID | GSGPAAKK | AAIRPRIHTKI | LKDCRQLPS |
| | | ID | QSVKM | DPLIDKYSSN | NO: 235) | KKLDGSGE | ANHESAFTL | RQLNHNIV |
| | | NO: 232) | PTRILER | VSIPKPSYKTL | | YVVDAANG | RSVIETKLTR | LKQVLAYFT |
| | | | VEIDHT | IRWQKNFTK | | ELKFVQQIF | MSSESDGLN | RLIATVPSS |
| | | | PLDLILL | SDGNLISLVD | | NELTIEQLL | VYLPEW | AKGNIGDLL |
| | | | HDDLLI | KNYLKGNRV | | G* (SEQ ID | (SEQ ID | LSPLEASTLL |
| | | | PLGRAY | ARKTGDEAF | | NO: 236) | NO: 237) | SCTTDEVYR |
| | | | LTLLVD | YERALERFLD | | | | LYEFGEIKA |
| | | | VFSGCII | SVRPSISAAY | | | | AIRPRIHTKI |
| | | | GFHLGF | QFYCDEITIA | | | | ANHESAFTL |
| | | | NAPSYV | NEQVISGQV | | | | RSVIETKLTR |
| | | | SVSKAII | PIVSYQTFKK | | | | MSSESDGL |
| | | | HSIKNK | RIKKEQPYNI | | | | NVYLPEW |
| | | | DYISNLP | VLARHGKYY | | | | (SEQ ID |
| | | | IKFENE | ADKLYHYYQ | | | | NO: 238) |
| | | | WLCNG | SVKMPTRILE | | | | |
| | | | KIENLV | RVEIDHTPLD | | | | |
| | | | VDNGP | LILLHDDLLIP | | | | |
| | | | EFWSKS | LGRAYLTLLV | | | | |
| | | | LDDACT | DVFSGCIIGF | | | | |
| | | | ECGINIT | HLGFNAPSY | | | | |
| | | | FNRVKK | VSVSKAIIHSI | | | | |
| | | | PWLKPF | KNKDYISNLP | | | | |
| | | | IERKFGE | IKFENEWLC | | | | |
| | | | IIQGIVG | NGKIENLVV | | | | |
| | | | WVPGK | DNGPEFWS | | | | |
| | | | TFSNVL | KSLDDACTE | | | | |
| | | | EKEDYK | CGINITFNRV | | | | |
| | | | PDKDAV | KKPWLKPFIE | | | | |
| | | | MRFSVF | RKFGEIIQGI | | | | |
| | | | VEELHR | VGWVPGKT | | | | |
| | | | WIVDV | FSNVLEKEDY | | | | |
| | | | HNAKA | KPDKDAVM | | | | |
| | | | DSRHTR | RFSVFVEELH | | | | |
| | | | IPNLSW | RWIVDVHN | | | | |
| | | | KNSFEC | AKADSRHTRI | | | | |
| | | | LPTKQL | PNLSWKNSF | | | | |
| | | | SADQEK | ECLPTKQLSA | | | | |
| | | | SFSITM | DQEKSFSIT | | | | |
| | | | GLLHIG | MGLLHIGTL | | | | |
| | | | TLTSKGI | TSKGIKYKHL | | | | |
| | | | KYKHLE | EYDSVALEQ | | | | |
| | | | YDSVAL | YRKQYPQTK | | | | |
| | | | EQYRKQ | ESKKKKIKIDP | | | | |
| | | | YPQTKE | DDLSTIFVFL | | | | |
| | | | SKKKKIK | EELSIYIEVPS | | | | |
| | | | IDPDDL | KNADGYTDK | | | | |
| | | | STIFVFL | LSLCVHQRL | | | | |
| | | | EELSIYIE | VKIHREYIKG | | | | |
| | | | VPSKNA | EINALSLAKA | | | | |
| | | | DGYTDK | RIALHERIQS | | | | |
| | | | LSLCVH | EQANLKAMS | | | | |
| | | | QRLVKI | LPERKRKAK | | | | |
| | | | HREYIK | GTKKAAKLT | | | | |
| | | | GEINAL | GLNSDSSSRT | | | | |
| | | | SLAKARI | SVNDISMVN | | | | |
| | | | ALHERI | EQESSLTKVE | | | | |
| | | | QSEQA | PIDDFRSKW | | | | |
| | | | NLKAM | NQRRKERSS | | | | |
| | | | RKAKGT | * (SEQ ID | | | | |
| | | | KKAAKL | NO: 234) | | | | |
| | | | TGLNSD | | | | | |
| | | | SSSRTS | | | | | |
| | | | VNDISM | | | | | |
| | | | VNEQES | | | | | |
| | | | SLTKVE | | | | | |
| | | | PIDDFR | | | | | |
| | | | SKWNQ | | | | | |
| | | | RRKERS | | | | | |
| | | | S (SEQ | | | | | |
| | | | ID | | | | | |
| | | | NO: 233) | | | | | |
| 41 | V. | MYVRN | MSFGPF | MYVRNLRKP | MTLLQPTNN | MTLLQPTN | MDTEIEVYP | MPKKKRKV |
| | flu- | LRKPSA | EDEFGSI | SANKNVYKF | DVDTLLADF | NDVDTLLA | DESLESFLLRL | GSGEQKLIS |
| | vialis | NKNVYK | TNDVQ | VSLKNGCTI | HQSFVVYPD | DFHQSFVV | SKYQGYERFS | EEDLEQKLI |
| | strain | FVSLKN | QQYDA | MCESSLEYD | VEKVFEGLD | YPDVEKVFE | HFAEDIWQS | SEEDLEQKL |
| | FDAARGQS | GCTIMC | SPEAKL | CCYYLEYSDD | WIVRRSQFG | GLDWIVRR | TIQQHQAIS | ISEEDLGSG |
| | _104 | ESSLEYD | SRLKYSP | VVRYQSQPK | KFAPSMLITG | SQFGKFAPS | GAFPFELSRI | DTEIEVYPD |
| | | CCYYLE | LESSKVI | GYRFPYRGK | GTGAGKTSV | MLITGGTG | NIYKAQTTS | ESLESFLLRL |
| | | YSDDVV | ERDLSS | QHPYTPDFL | VETYLNNHF | AGKTSVVE | QMRVRVLID | SKYQGYERF |
| | | RYQSQP | FPEEQK | VHKKDGTSY | SASEVLVTRV | TYLNNHFS | LERRLKLSDF | SHFAEDIW |
| | | KGYRFP | LKALER | LLEVKPLSKT | RPSFVETLV | ASEVLVTRV | GILRLALAHS | QSTIQQHQ |
| | | YRGKQ | YKLISLIA | FSSEFQDMF | WAIEKLNVP | RPSFVETLV | NANFSSDYK | AISGAFPFE |
| | | HPYTPD | KEINGG | HQKQIMASE | YNSRSKRSEI | WAIEKLNV | AVHRYGVDY | LSRINIYKA |
| | | FLVHKK | WTPKN | LGVPLLLVTD | GLQDYFISSV | PYNSRSKRS | PQAFLRKRFI | QTTSQMRV |
| | | DGTSYL | LIPLIDK | RQIRNDVHL | KKSKLKLLVIE | EIGLQDYFIS | PVCPKCLDE | RVLIDLERR |
| | | LEVKPLS | HIEKLSI | NNLKLVHRY | EAQELFECAS | SVKKSKLKL | APYIRQLWH | LKLSDFGILR |
| | | KTFSSEF | PKPSDR | SGFIENSSHL | PKERQKIRDR | LVIEEAQEL | FVPYQACHK | LALAHSNA |
| | | QDMFH | TVKRW | ESVWSAVSQ | LKMISDECRL | FECASPKER | HHGQLVQR | NFSSDYKA |
| | | QKQIM | YKAFCE | SSSICIKALPEI | PIVFIGIPTAK | QKIRDRLK | CPECGKLFDY | VHRYGVDY |
| | | ASELGV | SDGDIK | LNLTIGEVFA | LILEDSQWD | MISDECRLP | QSSELIEHCE | PQAFLRKRF |
| | | PLLLVT | SLVDSH | SVLRLIGLGK | RRIMVKRDL | IVFIGIPTAK | CGLSLTNIEP | PVCPKCLD |
| | | DRQIRN | HLKGNR | AKTKLDVLLD | PYIRITNEESL | LILEDSQW | EQESDSTFIV | EAPYIRQL |
| | | DVHLN | QPRIED | ENSLISVAGS | DIYIALLEGLE | DRRIMVKR | ARWLAGEKY | WHFVPYQ |
| | | NLKLVH | DEPLFIE | GPKKKRKVG | KTLSISVVPEL | DLPYIRITNE | IEPGLMSQQ | ACHKHHG |
| | | RYSGFIE | AVERFL | SGYPYDVPD | SDMDMAM | ESLDIYIALL | LTLSSRYGFLL | QLVQRCPE |
| | | NSSHLE | DAVRPS | YAYPYDVPD | RLLAASKGM | EGLEKTLSIS | WYINRYSEL | CGKLFDYQ |
| | | SVWSA | YSKAYQ | YAYPYDVPD | IGLIKELVGY | VVPELSDM | DEISFDNFVE | SSELIEHCEC |
| | | VSQSSSI | VYCDRI | YAGSGSFGP | ALELALLEGK | DMAMRLL | CCKTWPQKL | GLSLTNIEP |
| | | CIKALPE | EIENSSI | FEDEFGSITN | RQITQNEFIQ | AASKGMIG | DADLDSIVLK | EQESDSTFI |
| | | ILNLTIG | VSGEIA | DVQQQYDA | AFKSIFGPDIS | LIKELVGYA | ADIVRTRTW | VARWLAGE |
| | | EVFASV | KVSYEA | SPEAKLSRLK | NPFEIELDKL | LELALLEGK | SKTYFGEVFG | KYIEPGLMS |
| | | LRLIGLG | FKKRIKK | YSPLESSKVIE | LISQIIEYEGYI | RQITQNEFI | PLLKECRNLP | QQLTLSSRY |
| | | KAKTKL | LPPYTIA | RDLSSFPEEQ | LDSDSGDIKF | QAFKSIFGP | SRELSKNPVL | GFLLWYINR |
| | | DVLLDE | LKRHGK | KLKALERYKLI | THQIFEDIPL | DISNPFEIEL | QSIVQYFSRL | YSELDEISFD |
| | | NSLISVA | YYADKL | SLIAKEINGG | TELLR (SEQ | DKLLISQIIE | VANYPRDRT | NFVECCKT |
| | | (SEQ ID | FNYYEA | WTPKNLIPLI | ID NO: 242) | YEGSGPAA | ANIGDVLVS | WPQKLDA |
| | | NO: 239) | VKMPT | DKHIEKLSIPK | | KKKKLDGS | PLEASTLVSC | DLDSIVLKA |
| | | | RILERVE | PSDRTVKRW | | GGYILDSDS | STDEIYRLYQ | DIVRTRTW |
| | | | IDHTPL | YKAFCESDG | | GDIKFTHQI | FGELKAQLTP | SKTYFGEVF |
| | | | DLILLDD | DIKSLVDSHH | | FEDIPLTELL | KLHTKIENHH | GPLLKECRN |
| | | | ELLVPL | LKGNRQPRI | | R* (SEQ ID | SVFTLRSIIEL | LPSRELSKN |
| | | | GRAYLT | EDDEPLFIEA | | NO: 243) | KFSRMCSET | PVLQSIVQY |
| | | | LLVDVF | VERFLDAVR | | | DGLNHYLPE | FSRLVANYP |
| | | | SGCIIGF | PSYSKAYQV | | | W (SEQ ID | RDRTANIG |
| | | | HLGFKA | YCDRIEIENS | | | NO: 244) | DVLVSPLEA |
| | | | PSYTAV | SIVSGEIAKV | | | | STLVSCSTD |
| | | | SKAIIHS | SYEAFKKRIK | | | | EIYRLYQFG |
| | | | VKSKEY | KLPPYTIALK | | | | ELKAQLTPK |
| | | | VNELPI | RHGKYYADK | | | | LHTKIENHH |
| | | | GLSNQ | LFNYYEAVK | | | | SVFTLRSIIE |
| | | | WICHG | MPTRILERVE | | | | LKFSRMCSE |
| | | | KIENLV | IDHTPLDLILL | | | | TDGLNHYL |
| | | | VDNGA | DDELLVPLG | | | | PEW (SEQ |
| | | | EFWSKS | RAYLTLLVDV | | | | ID NO: 245) |
| | | | LDQACI | FSGCIIGFHL | | | | |
| | | | EAGINII | GFKAPSYTA | | | | |
| | | | YNKVRK | VSKAIIHSVK | | | | |
| | | | PWLKPF | SKEYVNELPI | | | | |
| | | | VERKFG | GLSNQWICH | | | | |
| | | | ELIQGIV | GKIENLVVD | | | | |
| | | | GWIPG | NGAEFWSKS | | | | |
| | | | RTFSNV | LDQACIEAGI | | | | |
| | | | LEKEDY | NIIYNKVRKP | | | | |
| | | | DPQKD | WLKPFVERK | | | | |
| | | | AVMRF | FGELIQGIVG | | | | |
| | | | SVFVEE | WIPGRTFSN | | | | |
| | | | LHRWII | VLEKEDYDP | | | | |
| | | | DVHNA | QKDAVMRF | | | | |
| | | | SADSRH | SVFVEELHR | | | | |
| | | | TRIPNY | WIIDVHNAS | | | | |
| | | | HWKKS | ADSRHTRIP | | | | |
| | | | EEVMP | NYHWKKSEE | | | | |
| | | | PPALTE | VMPPPALTE | | | | |
| | | | RDEIQF | RDEIQFRVI | | | | |
| | | | RVIMGV | MGVVHKGA | | | | |
| | | | VHKGAL | LTSKGIKFKH | | | | |
| | | | TSKGIKF | LMYDNVALE | | | | |
| | | | KHLMY | HYRKQYPQS | | | | |
| | | | DNVALE | KDSRIKTIKID | | | | |
| | | | HYRKQY | PDDLSRIFVF | | | | |
| | | | PQSKDS | LEEREGYIEV | | | | |
| | | | RIKTIKI | PCKCDPLGY | | | | |
| | | | DPDDLS | TKKLSLCEHL | | | | |
| | | | RIFVFLE | RTVKVHRDFI | | | | |
| | | | EREGYIE | KGQVDSLSL | | | | |
| | | | VPCKCD | AKARQALHE | | | | |
| | | | PLGYTK | RIKQEHENLR | | | | |
| | | | KLSLCE | QMSLPQRA | | | | |
| | | | HLRTVK | KKAKNGKK | | | | |
| | | | VHRDFI | MAELAGVSS | | | | |
| | | | KGQVD | DSPKSITTDY | | | | |
| | | | SLSLAK | PIEDIIQPHES | | | | |
| | | | ARQALH | TPVDDLQSL | | | | |
| | | | ERIKQE | WNKRRALRK | | | | |
| | | | HENLRQ | SSK* (SEQ ID | | | | |
| | | | MSLPQ | NO: 241) | | | | |
| | | | RAKKAK | | | | | |
| | | | NGKKM | | | | | |
| | | | AELAGV | | | | | |
| | | | SSDSPK | | | | | |
| | | | SITTDYP | | | | | |
| | | | IEDIIQP | | | | | |
| | | | HESTPV | | | | | |
| | | | DDLQSL | | | | | |
| | | | WNKRR | | | | | |
| | | | ALRKSS | | | | | |
| | | | K (SEQ | | | | | |
| | | | ID | | | | | |
| | | | NO: 240) | | | | | |
| 42 | V. | MYIRNL | MVGRF | MYIRNLRKP | MERAQKPE | MERAQKPE | VETDIQLYPD | MPKKKRKV |
| | nat- | RKPSPN | HDEFEP | SPNKNIFKFS | GIVVTTARR | GIVVTTARR | ESLESFLLRLS | GSGEQKLIS |
| | riegens | KNIFKFS | ENNEDS | SLKNRDAVM | NLDRDEVLA | NLDRDEVL | QEQSYERFS | EEDLEQKLI |
| | strain | SLKNRD | DRKHEF | CEGSLEKDC | DYHDSFSVY | ADYHDSFS | HFAEDIWQ | SEEDLEQKL |
| | CCUG | AVMCE | LPETQT | CYHFEYDPD | PEVEKVLSGL | VYPEVEKVL | NTLLQHEAIS | ISEEDLGSG |
| | 16373 | GSLEKD | ERLKYS | VVRYESQPE | EWIIKRRKFG | SGLEWIIKR | GAFPFELSRI | ETDIQLYPD |
| | | CCYHFE | RLQSTQ | GFYYDFNGK | TFAPSMLLT | RKFGTFAPS | NIYKAQTTS | ESLESFLLRL |
| | | YDPDVV | HIERDLS | KRPYTPDFLV | AGTGAGKTA | MLLTAGTG | QMRVRVLID | SQEQSYERF |
| | | RYESQP | SYPEEQ | TYHDGTFEY | TINHFIEKNL | AGKTATIN | LEKQLGLTNF | SHFAEDIW |
| | | EGFYYD | KNKALE | VEVKPYSKTL | SRNEVLITRV | HFIEKNLSR | GVLRLALAH | QNTLLQHE |
| | | FNGKKR | RYKLLCL | SKTFKQEFSA | KPSLLETLLW | NEVLITRVK | SKASFSPEYK | AISGAFPFE |
| | | PYTPDF | VANELS | RKEAANRRG | MAKELGAYR | PSLLETLLW | AVHRFGVDY | LSRINIYKA |
| | | LVTYHD | GGWTP | VGLVLVTDK | NSRAKPSEIG | MAKELGAY | PQAFLRKRF | QTTSQMRV |
| | | GTFEYV | KNLTPLI | QIRDGYFLK | LTDCVIETSK | RNSRAKPSE | APVCSQCLE | RVLIDLEKQ |
| | | EVKPYS | EKHFDK | NTELVHRYS | RVGLKLLVIE | IGLTDCVIET | ESPYIRQLW | LGLTNFGVL |
| | | KTLSKTF | TRLTKK | GCIAGDELAI | ECQELFERTS | SKRVGLKLL | QFIPYQACH | RLALAHSKA |
| | | KQEFSA | PSYKSL | KVYSNLVAQ | HNQRQDIRD | VIEECQELF | KHHCKLVHQ | SFSPEYKAV |
| | | RKEAAN | QRWHN | NTMKISDLA | RLKMISDEC | ERTSHNQR | CPECGNRLE | HRFGVDYP |
| | | RRGVGL | SFVDSD | DSIGESFGRV | HLPIVFVGLH | QDIRDRLK | YQHSELIEHC | QAFLRKRF |
| | | VLVTDK | GSFTSL | FASVLRLIAV | SAGLILEDSQ | MISDECHLP | DCGFRLASC | APVCSQCL |
| | | QIRDGY | VDKNHL | GKAGADLDI | WNRRIMVR | IVFVGLHSA | QAETANHAS | EESPYIRQL |
| | | FLKNTE | KGNRG | AQLSESTTVS | RTLPYIKITDE | GLILEDSQ | LTVAQWLA | WQFIPYQA |
| | | LVHRYS | ARVVG | VRGSGPKKK | SAIDNYLDVL | WNRRIMV | GEEVDKSGIF | CHKHHCKL |
| | | GCIAGD | DEKYYD | RKVGSGYPY | QALEKTVPLP | RRTLPYIKIT | NQLLTQSSR | VHQCPECG |
| | | ELAIKVY | EALKMF | DVPDYAYPY | FKVPLTDVD | DESAIDNYL | FGFLLWYVN | NRLEYQHS |
| | | SNLVAQ | LDARRQ | DVPDYAYPY | FAMRLLSAS | DVLQALEK | RYGDVDNIS | ELIEHCDCG |
| | | NTMKIS | SIRAAH | DVPDYAGSG | KGILGEIKELI | TVPLPFKVP | LEDFVRCCET | FRLASCQAE |
| | | DLADSI | AFYCDR | VGRFHDEFE | AAALEVTLEK | LTDVDFAM | WPQRLNEDL | TANHASLT |
| | | GESFGR | ITVANE | PENNEDSDR | NKDCIDEED | RLLSASKGIL | DAIVEKADM | VAQWLAG |
| | | VFASVL | AIVAGRI | KHEFLPETQT | FAAVYEKIND | GEIKELIAA | LRIQPWHKT | EEVDKSGIF |
| | | RLIAVG | PKVSYE | ERLKYSRLQS | PNDINPFTV | ALEVTLEKN | YFCEVFSELL | NQLLTQSS |
| | | KAGADL | AFKDRI | TQIIERDLSSY | QIDALTIEQI | KDCIDEEDF | KECRHLPSRE | RFGFLLWY |
| | | DIAQLS | RKEEPY | PEEQKNKAL | ASYENYVTD | AAVYEKIND | IGKNPVLQS | VNRYGDVD |
| | | ESTTVS | SVALAR | ERYKLLCLVA | AETGELRFVK | PNDINPFTV | VVQYFTELVT | NISLEDFVR |
| | | VR (SEQ | HGKYYA | NELSGGWTP | QVFSKLSIQQ | QIDALTIEQI | KYPRTKAANI | CCETWPQR |
| | | ID | DKLFNY | KNLTPLIEKH | LVG (SEQ ID | ASYEGSGP | ADMLLSPLE | LNEDLDAIV |
| | | NO: 246) | YQSVE | FDKTRLTKKP | NO: 249) | AAKKKKLD | ASTLLSCSTD | EKADMLRI |
| | | | MPTRIL | SYKSLQRWH | | GSGNYVTD | EILRLYQFGQ | QPWHKTYF |
| | | | ERVEM | NSFVDSDGS | | AETGELRFV | LKAQFTPKLH | CEVFSELLK |
| | | | DHTPLD | FTSLVDKNHL | | KQVFSKLSI | GKIENHHSV | ECRHLPSRE |
| | | | LILLHDD | KGNRGARV | | QQLVG* | FILRSIIELKLS | IGKNPVLQS |
| | | | LMVPLG | VGDEKYYDE | | (SEQ ID | RMCSETDGL | VVQYFTELV |
| | | | RAHLTL | ALKMFLDAR | | NO: 250) | MHYLPEW | TKYPRTKAA |
| | | | LVDVFS | RQSIRAAHA | | | (SEQ ID | NIADMLLSP |
| | | | GCIIGFH | FYCDRITVAN | | | NO: 251) | LEASTLLSCS |
| | | | LGFKAP | EAIVAGRIPK | | | | TDEILRLYQ |
| | | | SYVSAS | VSYEAFKDRI | | | | FGQLKAQF |
| | | | RAVIHA | RKEEPYSVAL | | | | TPKLHGKIE |
| | | | TKSKTYI | ARHGKYYAD | | | | NHHSVFILR |
| | | | SEMPIV | KLFNYYQSVE | | | | SIIELKLSRM |
| | | | FNNEW | MPTRILERVE | | | | CSETDGLM |
| | | | LCEGKIE | MDHTPLDLI | | | | HYLPEW |
| | | | NLVVD | LLHDDLMVP | | | | (SEQ ID |
| | | | NGAEF | LGRAHLTLLV | | | | NO: 252) |
| | | | WSKSW | DVFSGCIIGF | | | | |
| | | | EDACLE | HLGFKAPSY | | | | |
| | | | VGINVV | VSASRAVIHA | | | | |
| | | | YNKVRK | TKSKTYISEM | | | | |
| | | | PWLKPF | PIVFNNEWL | | | | |
| | | | VERKFG | CEGKIENLVV | | | | |
| | | | EIVQGI | DNGAEFWS | | | | |
| | | | VGWVP | KSWEDACLE | | | | |
| | | | GKTFSN | VGINVVYNK | | | | |
| | | | VLEKED | VRKPWLKPF | | | | |
| | | | YRPEKD | VERKFGEIVQ | | | | |
| | | | AVMRF | GIVGWVPGK | | | | |
| | | | STFVEEF | TFSNVLEKED | | | | |
| | | | HRWIV | YRPEKDAVM | | | | |
| | | | DVHNV | RFSTFVEEFH | | | | |
| | | | NADSRY | RWIVDVHN | | | | |
| | | | KRIPNLY | VNADSRYKRI | | | | |
| | | | WKQSY | PNLYWKASY | | | | |
| | | | DVLPPL | DVLPPLKLLP | | | | |
| | | | KLLPDQ | DQEQAFSVV | | | | |
| | | | EQAFSV | MGILHHRKL | | | | |
| | | | VMGILH | TDKGIKFMH | | | | |
| | | | HRKLTD | LEYDCVALSD | | | | |
| | | | KGIKFM | YRKTYPQTN | | | | |
| | | | HLEYDC | ESSKKKIKVD | | | | |
| | | | VALSDY | PDDLSAIYVY | | | | |
| | | | RKTYPQ | LDELQGYVK | | | | |
| | | | TNESSK | VPSKDPIGYT | | | | |
| | | | KKIKVD | VRLSVCEHEK | | | | |
| | | | PDDLSA | ILAAHRTYIK | | | | |
| | | | IYVYLDE | GEMDVLSLA | | | | |
| | | | LQGYVK | KARLALHDRI | | | | |
| | | | VPSKDP | ESEQADLM | | | | |
| | | | IGYTVRL | QLTHNERKR | | | | |
| | | | SVCEHE | KAKSTKKIAEI | | | | |
| | | | KILAAH | SSVNSDTPH | | | | |
| | | | RTYIKGE | SKLSDRTPKP | | | | |
| | | | MDVLSL | NVSISESESN | | | | |
| | | | AKARLA | SDTTPLESFR | | | | |
| | | | LHDRIES | SKWNERKN | | | | |
| | | | EQADL | RRE* (SEQ | | | | |
| | | | MQLTH | ID NO: 248) | | | | |
| | | | NERKRK | | | | | |
| | | | AKSTKKI | | | | | |
| | | | AEISSV | | | | | |
| | | | NSDTPH | | | | | |
| | | | SKLSDR | | | | | |
| | | | TPKPNV | | | | | |
| | | | SISESES | | | | | |
| | | | NSDTTP | | | | | |
| | | | LESFRSK | | | | | |
| | | | WNERK | | | | | |
| | | | NRRE | | | | | |
| | | | (SEQ ID | | | | | |
| | | | NO: 247) | | | | | |

TABLE B

| | | Wild-type | Modified | Wild-type | Modified | Wild-type | Modified |
|---|---|---|---|---|---|---|---|
| # | Organism | Cas8/5 | Cas8/5 | Cas7 | Cas7 | Cas6 | Cas6 |
| 0 | Tn6900 | MHIEELLDIED | MPKKKRKVGS | MELCTHLSY | MPKKKRKV | MTENRYFFA | MPKKKRKV |
| | | HGERDRQLRR | GDYKDDDDK | SRSLSPGKAV | GSGELCTHL | IRYLSDDVDC | GSGTENRY |
| | | YLAPYSAEIGV | DYKDDDDKD | FFYKTAESDF | SYSRSLSPG | GLLAGRCISIL | FFAIRYLSD |
| | | DGAEKMALV | YKDDDDKGS | VPLRIEVAKIS | KAVFFYKTA | HGFRQAHP | DVDCGLLA |
| | | VLLNLTLKRDR | GHIEELLDIED | GQKCGYTEG | ESDFVPLRI | GIQIGVAFPE | GRCISILHG |
| | | VESLCDEGLA | HGERDRQLRR | FDANLKPKNI | EVAKISGQK | WSDRDLGRS | FRQAHPGI |
| | | RQLLSDEGHIT | YLAPYSAEIGV | ERYELAYSNP | CGYTEGFD | IAFVSTNKSL | QIGVAFPE |
| | | NCLHTVRWL | DGAEKMALV | QTIEACYVPP | ANLKPKNIE | LERFRERSYF | WSDRDLGR |
| | | HTHNLKYPDA | VLLNLTLKRDR | NVDELYCRFS | RYELAYSNP | QVMQADNF | SIAFVSTNK |
| | | RVSGERLIINA | VESLCDEGLA | LRVEANSMR | QTIEACYVP | FALSLVLEVP | SLLERFRER |
| | | PPLIPGVISSA | RQLLSDEGHIT | PYVCSNPDV | PNVDELYC | DTCQNVRFI | SYFQVMQA |
| | | GLPMRMGW | NCLHTVRWL | LRVMIGLAQ | RFSLRVEAN | RNQNLAKLF | DNFFALSLV |
| | | AHDSSDINLA | HTHNLKYPDA | AYQRLGGYN | SMRPYVCS | VGERRRRLA | LEVPDTCQ |
| | | KLFGTSFRYRD | RVSGERLIINA | ELARRYSAN | NPDVLRVM | RAKRRAKAR | NVRFIRNQ |
| | | DSTNLALQLV | PPLIPGVISSA | VLRGIWLWR | GLAQAYQ | GEAFQPHM | NLAKLFVGE |
| | | ARSKTWEQAL | GLPMRMGW | NQYTQGTKI | RLGGYNEL | PDETKVVGV | RRRRLARA |
| | | IGLGLTQQQL | AHDSSDINLA | EIKTSLGSTY | ARRYSANV | FHSVFMQSA | KRRAKARG |
| | | DIWCQLLASN | KLFGTSFRYRD | HIPDARRLS | LRGIWLWR | SSGQSYILHI | EAFQPHMP |
| | | LENNTFPTVV | DSTNLALQLV | WSGDWPEL | NQYTQGTK | QKHRYERSE | DETKVVGV |
| | | SPFSKQVRFLY | ARSKTWEQAL | EQKQLEQLT | IEIKTSLGST | DSGYSSYGL | FHSVFMQS |
| | | QGNYCVVTP | IGLGLTQQQL | SEMAKALSQ | YHIPDARRL | ASNDLYTGY | ASSGQSYIL |
| | | VVSHALLAQL | DIWCQLLASN | PDIFWFADV | SWSGDWP | VPDLGAIFST | HIQKHRYER |
| | | QNVVHEKKL | LENNTFPTVV | TASLKTGFC | ELEQKQLE | LF (SEQ ID | SEDSGYSSY |
| | | QCTYIHHDHP | SPFSKQVRFLY | QEIFPSQKFT | QLTSEMAK | NO: 257) | GLASNDLYT |
| | | ASVGSLVGAL | QGNYCVVTP | ERPDDHSVA | ALSQPDIF | | GYVPDLGAI |
| | | GGKVAVLDYP | VVSHALLAQL | SRQLATVECS | WFADVTAS | | FSTLF (SEQ |
| | | PPVSPDKARS | QNVVHEKKL | DGQLAACIN | LKTGFCQEI | | ID NO: 258) |
| | | FSQARKHRLA | QCTYIHHDHP | PQKIGAALQ | FPSQKFTER | | |
| | | NGQSLFDRSV | ASVGSLVGAL | KIDDWWAN | PDDHSVAS | | |
| | | FNDHVFIDAL | GGKVAVLDYP | DADLPLRVH | RQLATVECS | | |
| | | KHVISRPGLTR | PPVSPDKARS | EYGANHEAL | DGQLAACI | | |
| | | KQQRQLRLSA | FSQARKHRLA | TALRHPATG | NPQKIGAA | | |
| | | LRYLRRQLAI | NGQSLFDRSV | QDFYHLLTK | LQKIDDW | | |
| | | WLGPIIEWRD | FNDHVFIDAL | AEQFVTVLES | WANDADL | | |
| | | EIVSSGRGEPG | KHVISRPGLTR | SEGGGVELP | PLRVHEYG | | |
| | | NLPSGGLELEL | KQQRQLRLSA | GEVHYLMAV | ANHEALTA | | |
| | | ITQPKKMLPEL | LRYLRRQLAI | LVKGGLFQK | LRHPATGQ | | |
| | | MLQVAGRFH | WLGPIIEWRD | GKGR (SEQ | DFYHLLTKA | | |
| | | LELQNHSAGR | EIVSSGRGEPG | ID NO: 255) | EQFVTVLES | | |
| | | RFAFHPALMA | NLPSGGLELEL | | SEGGGVEL | | |
| | | PIKSQILWLLR | ITQPKKMLPEL | | PGEVHYLM | | |
| | | QLADDEEKDE | MLQVAGRFH | | AVLVKGGL | | |
| | | PHPPTSCYYL | LELQNHSAGR | | FQKGKGR | | |
| | | HLSGLTVYDA | RFAFHPALMA | | (SEQ ID | | |
| | | SALANPYLCGI | PIKSQILWLLR | | NO: 256) | | |
| | | PSLSALAGFC | QLADDEEKDE | | | | |
| | | HDYERRLQSLI | PHPPTSCYYL | | | | |
| | | GQSVYFRGLA | HLSGLTVYDA | | | | |
| | | WYLGRYSLVT | SALANPYLCGI | | | | |
| | | GKHLPEPSKS | PSLSALAGFC | | | | |
| | | ADPKSVSAIRR | HDYERRLQSLI | | | | |
| | | PGLLDGRYCD | GQSVYFRGLA | | | | |
| | | LGMDLIIEVHI | WYLGRYSLVT | | | | |
| | | PTGGSLPFTTC | GKHLPEPSKS | | | | |
| | | LDLLRVALPAR | ADPKSVSAIRR | | | | |
| | | FAGGCLHPPS | PGLLDGRYCD | | | | |
| | | LYEEYNWCTV | LGMDLIIEVHI | | | | |
| | | YQDKSTLFTVL | PTGGSLPFTTC | | | | |
| | | SRLPRYGCWI | LDLLRVALPAR | | | | |
| | | YPSDADLRSFE | FAGGCLHPPS | | | | |
| | | ELSEALALDRR | LYEEYNWCTV | | | | |
| | | LRPVATGFVFL | YQDKSTLFTVL | | | | |
| | | EEPVERAGSIE | SRLPRYGCWI | | | | |
| | | GQHVYAESAI | YPSDADLRSFE | | | | |
| | | GTALCINPVE | ELSEALALDRR | | | | |
| | | MRLAGKKRFF | LRPVATGFVFL | | | | |
| | | GAGFWQLND | EEPVERAGSIE | | | | |
| | | AKGAILMNGS | GQHVYAESAI | | | | |
| | | ANTG (SEQ ID | GTALCINPVE | | | | |
| | | NO: 253) | MRLAGKKRFF | | | | |
| | | | GAGFWQLND | | | | |
| | | | AKGAILMNGS | | | | |
| | | | ANTG (SEQ ID | | | | |
| | | | NO: 254) | | | | |
| 1 | Tn6677 | MQTLKELIAS | MPKKKRKVGS | MKLPTNLAY | MPKKKRKV | MKWYYKTIT | MPKKKRKV |
| | | NPDDLTTELK | GDYKDDDDK | ERSIDPSDVC | GSGKLPTNL | FLPELCNNES | GSGKWYYK |
| | | RAFRPLTPHIA | DYKDDDDKD | FFVVWPDD | AYERSIDPS | LAAKCLRVLH | TITFLPELCN |
| | | IDGNELDALTI | YKDDDDKGS | RKTPLTYNSR | DVCFFVVW | GFNYQYETR | NESLAAKCL |
| | | LVNLTDKTDD | GQTLKELIASN | TLLGQMEAA | PDDRKTPLT | NIGVSFPLW | RVLHGFNY |
| | | QKDLLDRAKC | PDDLTTELKR | SLAYDVSGQ | YNSRTLLGQ | CDATVGKKIS | QYETRNIG |
| | | KQKLRDEKW | AFRPLTPHIAI | PIKSATAEAL | MEAASLAY | FVSKNKIELD | VSFPLWCD |
| | | WASCINCVNY | DGNELDALTIL | AQGNPHQV | DVSGQPIKS | LLLKQHYFV | ATVGKKISF |
| | | RQSHNPKFPD | VNLTDKTDDQ | DFCHVPYGA | ATAEALAQ | QMEQLQYF | VSKNKIELD |
| | | IRSEGVIRTQA | KDLLDRAKCK | SHIECSFSVS | GNPHQVDF | HISNTVLVPE | LLLKQHYFV |
| | | LGELPSFLLSSS | QKLRDEKWW | FSSELRQPYK | CHVPYGAS | DCTYVSFRR | QMEQLQYF |
| | | KIPPYHWSYS | ASCINCVNYR | CNSSKVKQT | HIECSFSVSF | CQSIDKLTAA | HISNTVLVP |
| | | HDSKYVNKSA | QSHNPKFPDI | LVQLVELYET | SSELRQPYK | GLARKIRRLE | EDCTYVSFR |
| | | FLTNEFCWDG | RSEGVIRTQAL | KIGWTELAT | CNSSKVKQ | KRALSRGEQ | RCQSIDKLT |
| | | EISCLGELLKD | GELPSFLLSSS | RYLMNICNG | TLVQLVELY | FDPSSFAQK | AAGLARKIR |
| | | ADHPLWNTL | KIPPYHWSYS | KWLWKNTR | ETKIGWTEL | EHTAIAHYHS | RLEKRALSR |
| | | KKLGCSQKTC | HDSKYVNKSA | KAYCWNIVL | ATRYLMNI | LGESSKQTN | GEQFDPSSF |
| | | KAMAKQLADI | FLTNEFCWDG | TPWPWNGE | CNGKWLW | RNFRLNIRM | AQKEHTAI |
| | | TLTTINVTLAP | EISCLGELLKD | KVGFEDIRTN | KNTRKAYC | LSEQPREGN | AHYHSLGE |
| | | NYLTQISLPDS | ADHPLWNTL | YTSRQDFKN | WNIVLTPW | SIFSSYGLSNS | SSKQTNRN |
| | | DTSYISLSPVA | KKLGCSQKTC | NKNWSAIVE | PWNGEKV | ENSFQPVPLI | FRLNIRMLS |
| | | SLSMQSHFH | KAMAKQLADI | MIKTAFSSTD | GFEDIRTNY | (SEQ ID | EQPREGNSI |
| | | QRLQDENRH | TLTTINVTLAP | GLAIFEVRAT | TSRQDFKN | NO: 263) | FSSYGLSNS |
| | | SAITRFSRTTN | NYLTQISLPDS | LHLPTNAMV | NKNWSAIV | | ENSFQPVPL |
| | | MGVTAMTCG | DTSYISLSPVA | RPSQVFTEKE | EMIKTAFSS | | I (SEQ ID |
| | | GAFRMLKSG | SLSMQSHFH | SGSKSKSKTQ | TDGLAIFEV | | NO: 264) |
| | | AKFSSPPHHR | QRLQDENRH | NSRVFQSTTI | RATLHLPTN | | |
| | | LNSKRSWLTS | SAITRFSRTTN | DGERSPILGA | AMVRPSQV | | |
| | | EHVQSLKQYQ | MGVTAMTCG | FKTGAAIATI | FTEKESGSK | | |
| | | RLNKSLIPENS | GAFRMLKSG | DDWYPEATE | SKSKTQNSR | | |
| | | RIALRRKYKIEL | AKFSSPPHHR | PLRVGRFGV | VFQSTTIDG | | |
| | | QNMVRSWF | LNSKRSWLTS | HREDVTCYR | ERSPILGAF | | |
| | | AMQDHTLDS | EHVQSLKQYQ | HPSTGKDFF | KTGAAIATI | | |
| | | NILIQHLNHDL | RLNKSLIPENS | SILQQAEHYI | DDWYPEAT | | |
| | | SYLGATKRFAY | RIALRRKYKIEL | EVLSANKTP | EPLRVGRF | | |
| | | DPAMTKLFTE | QNMVRSWF | AQETINDMH | GVHREDVT | | |
| | | LLKRELSNSIN | AMQDHTLDS | FLMANLIKG | CYRHPSTG | | |
| | | NGEQHTNGS | NILIQHLNHDL | GMFQHKGD | KDFFSILQQ | | |
| | | FLVLPNIRVCG | SYLGATKRFAY | (SEQ ID | AEHYIEVLS | | |
| | | ATALSSPVTV | DPAMTKLFTE | NO: 261) | ANKTPAQE | | |
| | | GIPSLTAFFGF | LLKRELSNSIN | | TINDMHFL | | |
| | | VHAFERNINR | NGEQHTNGS | | MANLIKGG | | |
| | | TTSSFRVESFA | FLVLPNIRVCG | | MFQHKGD | | |
| | | ICVHQLHVEK | ATALSSPVTV | | (SEQ ID | | |
| | | RGLTAEFVEK | GIPSLTAFFGF | | NO: 262) | | |
| | | GDGTISAPAT | VHAFERNINR | | | | |
| | | RDDWQCDVV | TTSSFRVESFA | | | | |
| | | FSLILNTNFAQ | ICVHQLHVEK | | | | |
| | | HIDQDTLVTSL | RGLTAEFVEK | | | | |
| | | PKRLARGSAKI | GDGTISAPAT | | | | |
| | | AIDDFKHINSF | RDDWQCDVV | | | | |
| | | STLETAIESLPI | FSLILNTNFAQ | | | | |
| | | EAGRWLSLYA | HIDQDTLVTSL | | | | |
| | | QSNNNLSDLL | PKRLARGSAKI | | | | |
| | | AAMTEDHQL | AIDDFKHINSF | | | | |
| | | MASCVGYHLL | STLETAIESLPI | | | | |
| | | EEPKDKPNSL | EAGRWLSLYA | | | | |
| | | RGYKHAIAECI | QSNNNLSDLL | | | | |
| | | IGLINSITFSSE | AAMTEDHQL | | | | |
| | | TDPNTIFWSL | MASCVGYHLL | | | | |
| | | KNYQNYLVV | EEPKDKPNSL | | | | |
| | | QPRSINDETT | RGYKHAIAECI | | | | |
| | | DKSSL (SEQ | IGLINSITFSSE | | | | |
| | | ID NO: 259) | TDPNTIFWSL | | | | |
| | | | KNYQNYLVV | | | | |
| | | | QPRSINDETT | | | | |
| | | | DKSSL (SEQ | | | | |
| | | | ID NO: 260) | | | | |
| 2 | Tn7005 | MTKLSDLLAIE | MPKKKRKVGS | MELCTQLNY | MPKKKRKV | MSQRYYFLIR | MPKKKRKV |
| | | DEAIKQTALKK | GDYKDDDDK | VRSLSAGKA | GSGELCTQL | YTNANADYG | GSGSQRYY |
| | | MFMPYTEDV | DYKDDDDKD | YFYYLSESGE | NYVRSLSA | LLAGRCISQ | FLIRYTNAN |
| | | CVDGYEQETL | YKDDDDKGS | MCPLDVDRT | GKAYFYYLS | MHLFMVNH | ADYGLLAG |
| | | TILLNLSSSHQ | GTKLSDLLAIE | RLRAPKGSYS | ESGEMCPL | HQAMNRVG | RCISQMHL |
| | | ADRCSDWLD | DEAIKQTALKK | EAYKGNKFV | DVDRTRLR | VSFPDWNES | FMVNHHQ |
| | | VARAQRYLKD | MFMPYTEDV | DKNVAPQDL | APKGSYSEA | SVGQTIAFVS | AMNRVGV |
| | | RENLDASLAEI | CVDGYEQETL | AYSNPQFIEE | YKGNKFVD | EDKEMMIGL | SFPDWNES |
| | | QWFHTHNLK | TILLNLSSSHQ | CYVKPGVDEI | KNVAPQDL | SFQPYFSLM | SVGQTIAFV |
| | | FPDCRVKDQR | ADRCSDWLD | YCAFSLRIRA | AYSNPQFIE | VNEGLFEISS | SEDKEMMI |
| | | IIARPLSTAEEF | VARAQRYLKD | NSLTPDMCS | ECYVKPGV | VYEVPDTSA | GLSFQPYFS |
| | | ISSAVLDQRLG | RENLDASLAEI | DDEVRSKLS | DEIYCAFSL | EVRFVRNQT | LMVNEGLF |
| | | WAHNSAVYR | QWFHTHNLK | MLAKIYKDL | RIRANSLTP | IGKNFLGSKK | EISSVYEVP |
| | | HTLWLLNPFK | FPDCRVKDQR | NGYKELAHR | DMCSDDEV | RRIKRSMAR | DTSAEVRFV |
| | | WQSQPVCILL | IIARPLSTAEEF | YAKNILLGT | RSKLSMLA | AELFGVEQSL | RNQTIGKN |
| | | LIQQKNPVWL | ISSAVLDQRLG | WLWRNREC | KIYKDLNGY | PVTNEDRVI | FLGSKKRRI |
| | | DLLTEFGLDVK | WAHNSAVYR | RNITIEVTTSE | KELAHRYAK | DSFHRIPISS | KRSMARAE |
| | | SLARLQRAIEE | HTLWLLNPFK | LDTFVVEHA | NILLGTWL | GSSRQDFILF | LFGVEQSLP |
| | | QLPENSFPDS | WQSQPVCILL | QKLSWYGH | WRNRECR | IQKELADERA | VTNEDRVI |
| | | VSTYSKQLRFP | LIQQKNPVWL | WDGDSTECL | NITIEVTTSE | KSGFNSYGF | DSFHRIPISS |
| | | WGDDYVSITP | DLLTEFGLDVK | ERLTAYLERA | LDTFVVEH | ATNQEKRAT | GSSRQDFIL |
| | | VVSHALQCEL | SLARLQRAIEE | LSDPTEYFY | AQKLSWYG | VPDLRFNLFE | FIQKELADE |
| | | EIRARSPENKF | QLPENSFPDS | MDVKAKMR | HWDGDST | EDSF (SEQ | RAKSGENS |
| | | SFVSSSLPNSA | VSTYSKQLRFP | VGWGDEVY | ECLERLTAY | ID NO: 269) | YGFATNQE |
| | | SIGNLCGSLG | WGDDYVSITP | PSQEFLDSRE | LERALSDPT | | KRATVPDL |
| | | GYMRVLNYPL | VVSHALQCEL | DGIPTKQLAT | EYFYMDVK | | RFNLFEEDS |
| | | GVKQAKGGTL | EIRARSPENKF | VELLSGKETV | AKMRVGW | | F (SEQ ID |
| | | TENRQKSGHY | SFVSSSLPNSA | AFHGQKVG | GDEVYPSQ | | NO: 270) |
| | | FDDYQVTNAK | SIGNLCGSLG | AALQSIDDW | EFLDSREDG | | |
| | | ICQVLNRLIGS | GYMRVLNYPL | WNENADKP | IPTKQLATV | | |
| | | EPSKTQRQRE | GVKQAKGGTL | LRVNEYGAD | ELLSGKETV | | |
| | | RARKVRSKILR | TENRQKSGHY | REYVIARRHV | AFHGQKVG | | |
| | | KQIALWMLPL | FDDYQVTNAK | THGNDFYQL | AALQSIDD | | |
| | | IELRDIAESEP | ICQVLNRLIGS | VRNTENWIE | WWNENAD | | |
| | | NQQQLEHDD | EPSKTQRQRE | TMTASRTIP | KPLRVNEY | | |
| | | TLAQAFLSLPE | RARKVRSKILR | NDVHFIMSV | GADREYVIA | | |
| | | WELGSLAGEF | KQIALWMLPL | LIKGGLFNCA | RRHVTHGN | | |
| | | NRRLHLAFQN | IELRDIAESEP | KAN (SEQ ID | DFYQLVRN | | |
| | | NIYSAKFAYHP | NQQQLEHDD | NO: 267) | TENWIETM | | |
| | | KLMQVAKAQ | TLAQAFLSLPE | | TASRTIPND | | |
| | | VTWVLEQLSK | WELGSLAGEF | | VHFIMSVLI | | |
| | | PINNQDTVTG | NRRLHLAFQN | | KGGLFNCA | | |
| | | EQYIYLSSMR | NIYSAKFAYHP | | KAN (SEQ | | |
| | | VQDAVAMSN | KLMQVAKAQ | | ID NO: 268) | | |
| | | PCLCGVPSLTA | VTWVLEQLSK | | | | |
| | | IWGFMHDYQ | PINNQDTVTG | | | | |
| | | RQFNQLVNN | EQYIYLSSMR | | | | |
| | | DSPVEFSSFAF | VQDAVAMSN | | | | |
| | | YVRNENIQST | PCLCGVPSLTA | | | | |
| | | AKLTEPNSIAK | IWGFMHDYQ | | | | |
| | | ARTVSNAKRP | RQFNQLVNN | | | | |
| | | TIRSKRLADLEI | DSPVEFSSFAF | | | | |
| | | DLVIRVHSESR | YVRNENIQST | | | | |
| | | ISDFRSALKTA | AKLTEPNSIAK | | | | |
| | | LPVAFAGGAL | ARTVSNAKRP | | | | |
| | | YQPQLSTQIE | TIRSKRLADLEI | | | | |
| | | WLRTFTGRSE | DLVIRVHSESR | | | | |
| | | LFHVLKGLPAY | ISDFRSALKTA | | | | |
| | | GRWLYPSEKQ | LPVAFAGGAL | | | | |
| | | PTNFDELERLL | YQPQLSTQIE | | | | |
| | | TQDDDNLLVS | WLRTFTGRSE | | | | |
| | | LGYHLLEHPTK | LFHVLKGLPAY | | | | |
| | | RDNAITGCHA | GRWLYPSEKQ | | | | |
| | | YAENAIGLAK | PTNFDELERLL | | | | |
| | | RINPIEVRFSG | TQDDDNLLVS | | | | |
| | | RDHFLNHAF | LGYHLLEHPTK | | | | |
| | | WSIECSSETILI | RDNAITGCHA | | | | |
| | | KNYRD (SEQ | YAENAIGLAK | | | | |
| | | ID NO: 265) | RINPIEVRFSG | | | | |
| | | | RDHFLNHAF | | | | |
| | | | WSIECSSETILI | | | | |
| | | | KNYRD (SEQ | | | | |
| | | | ID NO: 266) | | | | |
| 3 | Tn7007 | MEFTDILIIQD | MPKKKRKVGS | MKLCNNLNY | MPKKKRKV | MLTHYFSITY | MPKKKRKV |
| | | VKERNRAFKV | GDYKDDDDK | TRSLSPGKAV | GSGKLCNN | VPDDCDNEL | GSGLTHYFS |
| | | AFAHYSSAIFI | DYKDDDDKD | FYYESKDGQ | LNYTRSLSP | LAGRCIAEFH | ITYVPDDCD |
| | | DDHEVEAITCL | YKDDDDKGS | MNPIKCEQT | GKAVFYYES | KFISSLRLIEN | NELLAGRCI |
| | | LNLCTPKTEDY | GEFTDILIIQD | HLRAPKAGF | KDGQMNPI | NSFAIGFPN | AEFHKFISSL |
| | | LDKTSASLFLN | VKERNRAFKV | SEAFNSDYST | KCEQTHLR | WSEQSIGNE | RLIENNSFAI |
| | | NHDNIQKCLD | AFAHYSSAIFI | KNTAPQDLS | APKAGFSE | FAIFSDNSEL | GFPNWSEQ |
| | | ELKWFHSHN | DDHEVEAITCL | FSNPQFIEEC | AFNSDYSTK | LSAIKYQPYF | SIGNEFAIFS |
| | | VKYPDCRVKG | LNLCTPKTEDY | YVPVGIDEIKI | NTAPQDLS | NLMKSEELFS | DNSELLSAI |
| | | QSIISLPIDSVS | LDKTSASLFLN | RFSLRIEANS | FSNPQFIEE | ITDIKPVPNN | KYQPYFNL |
| | | NTINSNVVPY | NHDNIQKCLD | LQPDKCSDI | CYVPVGIDE | LPQIRFIRNQ | MKSEELFSI |
| | | RLGWSHDSG | ELKWFHSHN | QIREILQAFA | IKIRFSLRIEA | SIGKIFIGSKK | TDIKPVPNN |
| | | KVNYTHFLLSC | VKYPDCRVKG | TKYKENGGY | NSLQPDKC | RRIQRSITRN | LPQIRFIRN |
| | | FKWRGVQTT | QSIISLPIDSVS | QELGERYAK | SDIQIREILQ | NKEHTPISNE | QSIGKIFIGS |
| | | LSQLFITDTLF | NTINSNVVPY | NLLSGTWL | AFATKYKEN | DREFDTFHK | KKRRIQRSI |
| | | WLDIIKKIQCN | RLGWSHDSG | WRNEHNLG | GGYQELGE | VSCSSKSKQ | TRNNKEHT |
| | | WTKKQTEQFI | KVNYTHFLLSC | TSISIKTTSNQ | RYAKNLLSG | QQFILHIQKD | PISNEDREF |
| | | HSIQKEMPAK | FKWRGVQTT | EFNINNAFKL | TWLWRNE | ITPRTTDSND | DTFHKVSCS |
| | | TLPEDISPYSK | LSQLFITDTLF | SRKTSAKDK | HNLGTSISIK | SYNSYGLAT | SKSKQQQFI |
| | | QILFPYKNDYL | WLDIIKKIQCN | KTISKLGSEIA | TTSNQEFNI | NSKHLGTVP | LHIQKDITP |
| | | TLTPVTSNSIQ | WTKKQTEQFI | SALSDPDHY | NNAFKLSR | DLSKIPFYCE | RTTDSNDS |
| | | TWLEHQSRKP | HSIQKEMPAK | YFADITATIN | KTSAKDKKT | DKLSNKDQ | YNSYGLAT |
| | | NDIRWIKRES | TLPEDISPYSK | VAFCQEIYPS | ISKLGSEIAS | (SEQ ID | NSKHLGTV |
| | | KHPASVGALS | QILFPYKNDYL | QEFLDTKEK | ALSDPDHY | NO: 275) | PDLSKIPFY |
| | | SSIGGYHSLLS | TLTPVTSNSIQ | GKPSKVYAK | YFADITATI | | CEDKLSNK |
| | | SLPSTSQSPHS | TWLEHQSRKP | TSLQTGEKTI | NVAFCQEIY | | DQ (SEQ ID |
| | | YHDNMTSKTE | NDIRWIKRES | AFHAQKIGA | PSQEFLDTK | | NO: 276) |
| | | CREAFCASAIT | KHPASVGALS | AIQLIDDWW | EKGKPSKVY | | |
| | | EKSTTDALQR | SSIGGYHSLLS | ADDADIPLR | AKTSLQTGE | | |
| | | LISSEVRMNV | SLPSTSQSPHS | VNEFGADH | KTIAFHAQK | | |
| | | KHRKQIRKSGI | YHDNMTSKTE | HNVIARRHP | IGAAIQLID | | |
| | | HFIRQKIALWL | CREAFCASAIT | SHRNDFYTLI | DWWADD | | |
| | | TPLIRWRDHI | EKSTTDALQR | QNADNYCA | ADIPLRVNE | | |
| | | DNNQIQITND | LISSEVRMNV | QLNENSDIT | FGADHHNV | | |
| | | HPSLVNLFLSS | KHRKQIRKSGI | DDMHYVMA | IARRHPSHR | | |
| | | PIANFPDLLTP | HFIRQKIALWL | VLVKGGLFQ | NDFYTLIQN | | |
| | | LHNHLNQTLG | TPLIRWRDHI | KSASSKKGK | ADNYCAQL | | |
| | | NNKYTKRFAY | DNNQIQITND | (SEQ ID | NENSDITD | | |
| | | HPDLMPIFKS | HPSLVNLFLSS | NO: 273) | DMHYVMA | | |
| | | QISWILNKLT | PIANFPDLLTP | | VLVKGGLF | | |
| | | QDENINQQP | LHNHLNQTLG | | QKSASSKK | | |
| | | VLTRTQFIHLK | NNKYTKRFAY | | GK (SEQ ID | | |
| | | NLRLYNGNAL | HPDLMPIFKS | | NO: 274) | | |
| | | SSPYVCGLPSL | QISWILNKLT | | | | |
| | | TGFWGFMHD | QDENINQQP | | | | |
| | | FERRLKTKIEE | VLTRTQFIHLK | | | | |
| | | NIHFEAFSLFV | NLRLYNGNAL | | | | |
| | | HQYELQSSPP | SSPYVCGLPSL | | | | |
| | | LCEASDVYKK | TGFWGFMHD | | | | |
| | | RELSPAKRLLT | FERRLKTKIEE | | | | |
| | | QPSYSCDMRF | NIHFEAFSLFV | | | | |
| | | DLIIKVHTEVN | HQYELQSSPP | | | | |
| | | LSDISQRMQS | LCEASDVYKK | | | | |
| | | AMPARCVGG | RELSPAKRLLT | | | | |
| | | TLHQPSLHESL | QPSYSCDMRF | | | | |
| | | EWLRTYTSSE | DLIIKVHTEVN | | | | |
| | | HLFEELACLPN | LSDISQRMQS | | | | |
| | | SGRWIYPPSE | AMPARCVGG | | | | |
| | | TFNTPDEFLSI | TLHQPSLHESL | | | | |
| | | LGNSTHLAIC | EWLRTYTSSE | | | | |
| | | NGYSFLEDPT | HLFEELACLPN | | | | |
| | | YRENVSLNQH | SGRWIYPPSE | | | | |
| | | VFCEPLIGLAE | TFNTPDEFLSI | | | | |
| | | QVIPIDMRLN | LGNSTHLAIC | | | | |
| | | RQKHYFSNAF | NGYSFLEDPT | | | | |
| | | WSINSDFNSIL | YRENVSLNQH | | | | |
| | | ISKA (SEQ ID | VFCEPLIGLAE | | | | |
| | | NO: 271) | QVIPIDMRLN | | | | |
| | | | RQKHYFSNAF | | | | |
| | | | WSINSDFNSIL | | | | |
| | | | ISKA (SEQ ID | | | | |
| | | | NO: 272) | | | | |
| 4 | Tn7009 | MLTINELLEIA | MPKKKRKVGS | MKIPTHLSY | MPKKKRKV | MRSYFYITYL | MPKKKRKV |
| | | DIEERNKAIRS | GDYKDDDDK | MRSLSPSPA | GSGKIPTHL | PENVNNELL | GSGRSYFYI |
| | | RLRPFHEPLN | DYKDDDDKD | LFFYKTDESD | SYMRSLSPS | AARCVNVLH | TYLPENVN |
| | | VDGSEKEILIV | YKDDDDKGS | FNPIEVFSEGI | PALFFYKTD | GFVAKEDVV | NELLAARC |
| | | LLNLGYSSKEQ | GLTINELLEIA | NGRMSGSA | ESDFNPIEV | DIGISFPAWS | VNVLHGFV |
| | | VDLLEQKSAQ | DIEERNKAIRS | VAYNKDGKL | FSEGINGR | EHTVGNQLA | AKEDVVDI |
| | | QFLKGEELFG | RLRPFHEPLN | KNVTANDLG | MSGSAVAY | FVSTSKSKLT | GISFPAWSE |
| | | KTISEAEWIHT | VDGSEKEILIV | HANLHASEY | NKDGKLKN | RILHHNYFS | HTVGNQLA |
| | | HNLKYPDIRVS | LLNLGYSSKEQ | CYVPPKIKEF | VTANDLGH | MMKEDGLF | FVSTSKSKL |
| | | KQTIRATLPED | VDLLEQKSAQ | YCKFSLTIAP | ANLHASEY | YISNIEPVPT | TRILHHNYF |
| | | VEGVCSKDILE | QFLKGEELFG | NSLSPYICND | CYVPPKIKE | GLKEIQFLRN | SMMKEDG |
| | | SIELGWSHNA | KTISEAEWIHT | QDLVMYLEK | FYCKFSLTIA | NTIAKTTLGE | LFYISNIEPV |
| | | TFVGKVTPLIT | HNLKYPDIRVS | LAQCYAEKG | PNSLSPYIC | KRRRNKRAF | PTGLKEIQF |
| | | EFKWQGKVT | KQTIRATLPED | GYQELATRY | NDQDLVM | ERAEARGDE | LRNNTIAKT |
| | | CLINLLLSESAF | VEGVCSKDILE | AKNILNGLW | YLEKLAQCY | YAPVQNNQ | TLGEKRRR |
| | | WVNLLITLGV | SIELGWSHNA | LWRNKKSPK | AEKGGYQE | AQFIHNYHIL | NKRAFERA |
| | | SKRWVNRTKI | TFVGKVTPLIT | VDISVYDFLS | LATRYAKNI | NCTSGSKN | EARGDEYA |
| | | QLADITANSF | EFKWQGKVT | EQEVANTAG | LNGLWLW | MSFPLYIQKR | PVQNNQA |
| | | PEEVDRYSPQ | CLINLLLSESAF | VQSLSWDG | RNKKSPKV | EDTSHQNCD | QFIHNYHIL |
| | | LRFYNQRGYV | WVNLLITLGV | NWGKYHDE | DISVYDFLS | FNHYGLASN | NCTSGSKN |
| | | SVTPVTNHKL | SKRWVNRTKI | LQKLSKIIAQ | EQEVANTA | KLYSGTVPEF | MSFPLYIQK |
| | | LSEIQKRCFNK | QLADITANSF | ALHNNEACE | GVQSLSWD | NFDQ (SEQ | REDTSHQN |
| | | EFRCRKVKHP | PEEVDRYSPQ | LEVVATIRNR | GNWGKYH | ID NO: 281) | CDFNHYGL |
| | | RATCAGHLITS | LRFYNQRGYV | FMQEIYPSQ | DELQKLSKII | | ASNKLYSGT |
| | | LGGYVSVLAY | SVTPVTNHKL | LLPEENKVHK | AQALHNNE | | VPEFNFDQ |
| | | YPDRGFNRNI | LSEIQKRCFNK | QLATTRVED | ACELEVVAT | | (SEQ ID |
| | | NQYIDDKTDS | EFRCRKVKHP | GSETTCLGRF | IRNRFMQEI | | NO: 282) |
| | | NFFNSKYLNN | RATCAGHLITS | KVGAAIQIID | YPSQLLPEE | | |
| | | HNFLEALGEL | LGGYVSVLAY | DWHGGDKP | NKVHKQLA | | |
| | | VFSPKRETLKL | YPDRGFNRNI | LRVSSYGSVP | TTRVEDGSE | | |
| | | TRIARVAAIKSI | NQYIDDKTDS | ERLVALRTPS | TTCLGRFKV | | |
| | | RQTLYWWLA | NFFNSKYLNN | NKKDVYSLLP | GAAIQIIDD | | |
| | | KATDYKKHAN | HNFLEALGEL | KIIDYINFLES | WHGGDKP | | |
| | | ISSDVSSNAKL | VFSPKRETLKL | NNLGENETS | LRVSSYGSV | | |
| | | FKRYLNQGES | TRIARVAAIKSI | NEINYLMA | PERLVALRT | | |
| | | KNELASELSNL | RQTLYWWLA | MLVKGDVL | PSNKKDVY | | |
| | | IHEQLAQANQ | KATDYKKHAN | GMGSEKKSK | SLLPKIIDYI | | |
| | | TKQFAYHSKLI | ISSDVSSNAKL | (SEQ ID | NFLESNNL | | |
| | | SPIKRQLQFLL | FKRYLNQGES | NO: 279) | GENETSNEI | | |
| | | KNRANSETEQ | KNELASELSNL | | NYLMAML | | |
| | | QEQRVFYLHL | IHEQLAQANQ | | VKGDVLG | | |
| | | KRLRVEDLETL | TKQFAYHSKLI | | MGSEKKSK | | |
| | | SCPYLWGMP | SPIKRQLQFLL | | (SEQ ID | | |
| | | SIIAFAGFAHK | KNRANSETEQ | | NO: 280) | | |
| | | FELNLKKLGFH | QEQRVFYLHL | | | | |
| | | NIRVMGVACF | KRLRVEDLETL | | | | |
| | | VHLYQVTAKT | SCPYLWGMP | | | | |
| | | SLPAYSHLKKE | SIIAFAGFAHK | | | | |
| | | KQSDQLRPTR | FELNLKKLGFH | | | | |
| | | PALVSAPKSQ | NIRVMGVACF | | | | |
| | | MLFDLVLRLW | VHLYQVTAKT | | | | |
| | | NGGNEYNLES | SLPAYSHLKKE | | | | |
| | | LPNPVQIREAL | KQSDQLRPTR | | | | |
| | | PTRYAGGTIFP | PALVSAPKSQ | | | | |
| | | TIRKLEERFTTS | MLFDLVLRLW | | | | |
| | | HNLTELFNSLS | NGGNEYNLES | | | | |
| | | FMPAKGCWL | LPNPVQIREAL | | | | |
| | | YPSQFKVHSL | PTRYAGGTIFP | | | | |
| | | DELHKALDTD | TIRKLEERFTTS | | | | |
| | | LNLRPVAIGY | HNLTELFNSLS | | | | |
| | | QYLEEPKYRD | FMPAKGCWL | | | | |
| | | GGISELHCYAE | YPSQFKVHSL | | | | |
| | | NLLGLTRCTN | DELHKALDTD | | | | |
| | | SVDVRVGGA | LNLRPVAIGY | | | | |
| | | QRFLREAFWA | QYLEEPKYRD | | | | |
| | | QKTTDSEVLM | GGISELHCYAE | | | | |
| | | VKSRFEFKL | NLLGLTRCTN | | | | |
| | | (SEQ ID | SVDVRVGGA | | | | |
| | | NO: 277) | QRFLREAFWA | | | | |
| | | | QKTTDSEVLM | | | | |
| | | | VKSRFEFKL | | | | |
| | | | (SEQ ID | | | | |
| | | | NO: 278) | | | | |
| 5 | Tn7011 | MNLQDAFAIE | MPKKKRKVGS | MQLPRHLSY | MPKKKRKV | MKRYYFTITY | MPKKKRKV |
| | | SLKEKTTALRK | GDYKDDDDK | TRSLSPSKAV | GSGQLPRH | LPKNCDVSLL | GSGKRYYFT |
| | | LFTPYMSHVA | DYKDDDDKD | FFYKTSESDF | LSYTRSLSPS | AGRCIGILHG | ITYLPKNCD |
| | | VDGFEEQALT | YKDDDDKGS | EPLQIEQNKL | KAVFFYKTS | FMSSREISNI | VSLLAGRCI |
| | | VLINLVYKRSEI | GNLQDAFAIE | VGQKSGFGD | ESDFEPLQI | GVCFPKWN | GILHGFMS |
| | | DDLTSTRTAK | SLKEKTTALRK | AYQKQNVA | EQNKLVGQ | EQEIGNELAF | SREISNIGV |
| | | SVLRDEVLLSK | LFTPYMSHVA | KNLAPQDLA | KSGFGDAY | VSTDKKQLT | CFPKWNEQ |
| | | CINEVKWFHT | VDGFEEQALT | FGNPQTIDV | QKQNVAK | NLSQQSYFE | EIGNELAFV |
| | | HNLKYPDIRVS | VLINLVYKRSEI | CYVPPAVNE | NLAPQDLA | MMAQDKLF | STDKKQLT |
| | | HQRLISKVVSE | DDLTSTRTAK | LFCRFSLRVE | FGNPQTID | GLSKILEVPT | NLSQQSYFE |
| | | DIAGICSRSLP | SVLRDEVLLSK | ANSNEPHVC | VCYVPPAV | NQNEVMFIR | MMAQDKL |
| | | LSFGWSHNSA | CINEVKWFHT | DDPKVIYWL | NELFCRFSL | NQSVAKAFV | FGLSKILEVP |
| | | EINHAKLFLTS | HNLKYPDIRVS | KRFFETYKKH | RVEANSNE | GEKQRRLKR | TNQNEVM |
| | | FTWQGEVTCL | HQRLISKVVSE | NGLNEVATR | PHVCDDPK | AKKRAEARG | FIRNQSVAK |
| | | ANLLINEEPV | DIAGICSRSLP | YAKNILMGN | VIYWLKRFF | EVYNPEYQF | AFVGEKQR |
| | | WINLIRTYGFT | LSFGWSHNSA | WLWRNRQS | ETYKKHNG | EAKDIGHFH | RLKRAKKRA |
| | | KKAVLGIAGKI | EINHAKLFLTS | PNVDIEILTE | LNEVATRY | SIPVSSKANG | EARGEVYN |
| | | KQLLPVAELPL | FTWQGEVTCL | HAAPIIVEGA | AKNILMGN | QSYVLHIQKI | PEYQFEAK |
| | | EVSSFSPQLQ | ANLLINEEPV | QKLKWQGN | WLWRNRQ | ENTNATENQ | DIGHFHSIP |
| | | MPFQQSYLA | WINLIRTYGFT | WQNNQTAL | SPNVDIEILT | FNNYGFATN | VSSKANGQ |
| | | VTPVVSHAML | KKAVLGIAGKI | ITLSEAIQEGL | EHAAPIIVE | QTFQGTVPS | SYVLHIQKIE |
| | | AKIQQLTTDR | KQLLPVAELPL | SNPQNYCYL | GAQKLKW | LNTQ (SEQ | NTNATENQ |
| | | KLNFGLVEHS | EVSSFSPQLQ | DITAKIKNAF | QGNWQN | ID NO: 287) | FNNYGFAT |
| | | RPANVGDLAS | MPFQQSYLA | SQEVHPSQK | NQTALITLS | | NQTFQGTV |
| | | SVGGNIRVLR | VTPVVSHAML | FVDNVEQG | EAIQEGLSN | | PSLNTQ |
| | | YFPKTYSKAV | AKIQQLTTDR | MSSKQLAYT | PQNYCYLDI | | (SEQ ID |
| | | NCSEVENNDS | KLNFGLVEHS | QVGDKKAAS | TAKIKNAFS | | NO: 288) |
| | | EKAFKIRALLN | RPANVGDLAS | LNSQKVGAA | QEVHPSQK | | |
| | | SQFQQALLVL | SVGGNIRVLR | IQTIDDWYE | FVDNVEQG | | |
| | | VGIKQFNTLR | YFPKTYSKAV | GGYKPLRTH | MSSKQLAY | | |
| | | QKRLARVAAI | NCSEVENNDS | EYGADKQIL | TQVGDKKA | | |
| | | RQVRVSLQL | EKAFKIRALLN | VAHRTPKSH | ASLNSQKV | | |
| | | WLDNILEAKN | SQFQQALLVL | SDFYSLLPRIA | GAAIQTIDD | | |
| | | NAQGQAYPE | VGIKQFNTLR | LHIKHMEKH | WYEGGYKP | | |
| | | WAKHYLDQSI | QKRLARVAAI | GLEQSEESN | LRTHEYGA | | |
| | | TNCISQFSNVL | RQVRVSLQL | AVHFIAAVLI | DKQILVAH | | |
| | | NESLGNLSKLK | WLDNILEAKN | KGGLFQRSK | RTPKSHSDF | | |
| | | RFAYHPNLM | NAQGQAYPE | A (SEQ ID | YSLLPRIALH | | |
| | | GVFKTQLNYV | WAKHYLDQSI | NO: 285) | IKHMEKHG | | |
| | | FTHCIPDEETL | TNCISQFSNVL | | LEQSEESNA | | |
| | | NDEQIVYVHC | NESLGNLSKLK | | VHFIAAVLI | | |
| | | QDMRVFDAE | RFAYHPNLM | | KGGLFQRS | | |
| | | AMANPYIQG | GVFKTQLNYV | | KA (SEQ ID | | |
| | | MPSLTALNGL | FTHCIPDEETL | | NO: 286) | | |
| | | AHNFERKLKN | NDEQIVYVHC | | | | |
| | | FIDPSIKCIGSA | QDMRVFDAE | | | | |
| | | INIESYQLHTG | AMANPYIQG | | | | |
| | | KPLPEPSKLKQ | MPSLTALNGL | | | | |
| | | VAGRSHVIRS | AHNFERKLKN | | | | |
| | | GIIDKPKCDITL | FIDPSIKCIGSA | | | | |
| | | DLVFRLFVPNI | INIESYQLHTG | | | | |
| | | KLLDKLNSQL | KPLPEPSKLKQ | | | | |
| | | VKPALPSMFA | VAGRSHVIRS | | | | |
| | | GGTMHPPSLY | GIIDKPKCDITL | | | | |
| | | QNIDWCHLH | DLVFRLFVPNI | | | | |
| | | TKPSELFKNIK | KLLDKLNSQL | | | | |
| | | AKSLNGSWLY | VKPALPSMFA | | | | |
| | | PSKKVVKSFE | GGTMHPPSLY | | | | |
| | | QLIDALNGNF | QNIDWCHLH | | | | |
| | | NLRPAAIGFA | TKPSELFKNIK | | | | |
| | | ALEEPIKRDVA | AKSLNGSWLY | | | | |
| | | LHEYHCYAEP | PSKKVVKSFE | | | | |
| | | VIGLLECVSNT | QLIDALNGNF | | | | |
| | | SVKYAGAKQF | NLRPAAIGFA | | | | |
| | | FHDAFWVMD | ALEEPIKRDVA | | | | |
| | | VQKESMLMK | LHEYHCYAEP | | | | |
| | | KSKFEYE (SEQ | VIGLLECVSNT | | | | |
| | | ID NO: 283) | SVKYAGAKQF | | | | |
| | | | FHDAFWVMD | | | | |
| | | | VQKESMLMK | | | | |
| | | | KSKFEYE (SEQ | | | | |
| | | | ID NO: 284) | | | | |
| 6 | Tn7014 | MTTLQDLIDIE | MPKKKRKVGS | MELCSQLNY | MPKKKRKV | MESRYYFSIR | MPKKKRKV |
| | | DSKLRFIEIKKA | GDYKDDDDK | VRSLSPGRAY | GSGELCSQL | YIPEHVDNEL | GSGESRYYF |
| | | FMPYTRPVEV | DYKDDDDKD | FYYLDEDNK | NYVRSLSPG | LAGRCISNM | SIRYIPEHV |
| | | DGSEKQALIVL | YKDDDDKGS | MRPLQIDRT | RAYFYYLDE | HGFLSHERN | DNELLAGR |
| | | LNLSLSKPEVK | GTTLQDLIDIE | HLRAPKSGY | DNKMRPL | TQFKNSVGI | CISNMHGF |
| | | DWLDFPRAL | DSKLRFIEIKKA | SEAFSGNFKS | QIDRTHLRA | CFPLWNEQT | LSHERNTQ |
| | | DYFADSDNLS | FMPYTRPVEV | KNIAPQDLSY | PKSGYSEAF | VGNVITFVST | FKNSVGICF |
| | | AAEQEIQWF | DGSEKQALIVL | SNPQFIEECY | SGNFKSKNI | NESILTGLSY | PLWNEQTV |
| | | HTHNLKFPDC | LNLSLSKPEVK | VPPGVNDIY | APQDLSYS | QPYFSTMM | GNVITFVST |
| | | RVSEQRIIATP | DWLDFPRAL | CAFSLRVRA | NPQFIEECY | NENLFEISGI | NESILTGLSY |
| | | LYTETPTLTSQ | DYFADSDNLS | NSLSPEVCV | VPPGVNDI | RIVPDDAKD | QPYFSTM |
| | | SLNRAYGWA | AAEQEIQWF | DNEVRDILC | YCAFSLRVR | VRFVFNKTIQ | MNENLFEIS |
| | | HNSAVYKHTI | HTHNLKFPDC | NFAALYKEL | ANSLSPEVC | KIFNGSKKRR | GIRIVPDDA |
| | | WLLNEFRWR | RVSEQRIIATP | GGYRELARR | VDNEVRDIL | IKRAMKRAE | KDVRFVFN |
| | | GRVENLLNLIC | LYTETPTLTSQ | YAKNILMGT | CNFAALYKE | EFGHTFTPIS | KTIQKIFNG |
| | | GGDDFWLELL | SLNRAYGWA | WVWRNREC | LGGYRELAR | VEVREFELFH | SKKRRIKRA |
| | | ADMGLKPKA | HNSAVYKHTI | RNIRVEVKTE | RYAKNILM | EIPINSKSSG | MKRAEEFG |
| | | QIQLKDLIEHQ | WLLNEFRWR | DKEWVITDA | GTWVWRN | RDFVLHIQR | HTFTPISVE |
| | | LPLTHFPDEV | GRVENLLNLIC | RFLDWYGS | RECRNIRVE | QNPVEAEIG | VREFELFHE |
| | | NRYSKQLRFP | GGDDFWLELL | WEKDSQLAL | VKTEDKEW | QGFNGYGFA | IPINSKSSGR |
| | | WRGDYLSVTP | ADMGLKPKA | DEFTDYLSQ | VITDARFLD | SNQLWRRT | DFVLHIQR |
| | | VVSHAIQQQL | QIQLKDLIEHQ | ALSDRTCYF | WYGSWEK | VPLILF (SEQ | QNPVEAEI |
| | | SVLSRQGECSL | LPLTHFPDEV | NMDIKAKLT | DSQLALDEF | ID NO: 293) | GQGFNGY |
| | | RFKTMTYPNS | NRYSKQLRFP | VGWGDEVY | TDYLSQALS | | GFASNQLW |
| | | ASIGNLCGSLG | WRGDYLSVTP | PSQEFLDVKE | DRTCYFNM | | RRTVPLILF |
| | | GYINVLNYPID | VVSHAIQQQL | AGKPSKLLAK | DIKAKLTVG | | (SEQ ID |
| | | VIANRHQTLG | SVLSRQGECSL | VTVNGEESA | WGDEVYPS | | NO: 294) |
| | | ASRSRTKRYF | RFKTMTYPNS | AFHSQKVGA | QEFLDVKE | | |
| | | DDFQLTSKST | ASIGNLCGSLG | AIQRIDDW | AGKPSKLLA | | |
| | | CSVLAHLTGFE | GYINVLNYPID | WDENADKP | KVTVNGEE | | |
| | | QPQMRKAQK | VIANRHQTLG | LRVNEYGAD | SAAFHSQK | | |
| | | HVRQYQLKIIR | ASRSRTKRYF | KEYAIARRHS | VGAAIQRID | | |
| | | KQIALWLLPLI | DDFQLTSKST | SRHRDFYSLI | DWWDENA | | |
| | | ELRDNSVTDPI | CSVLAHLTGFE | AHTESYVEL | DKPLRVNE | | |
| | | GFYDEPDDEL | QPQMRKAQK | MLETNLISD | YGADKEYAI | | |
| | | AKRFLTINELD | HVRQYQLKIIR | DVHFIMAVL | ARRHSSRH | | |
| | | FIELTTSLNQR | KQIALWLLPLI | TKGGVFSGA | RDFYSLIAH | | |
| | | LNIALQNNRF | ELRDNSVTDPI | SKKSKKDE | TESYVELML | | |
| | | ASRFAYHPKL | GFYDEPDDEL | (SEQ ID | ETNLISDDV | | |
| | | MRVLKTELIW | AKRFLTINELD | NO: 291) | HFIMAVLTK | | |
| | | VLTQLSQPEP | FIELTTSLNQR | | GGVFSGAS | | |
| | | EPPTVSDSKV | LNIALQNNRF | | KKSKKDE | | |
| | | QYLYLSSMRV | ASRFAYHPKL | | (SEQ ID | | |
| | | FDAAAMSCPY | MRVLKTELIW | | NO: 292) | | |
| | | LSGAPSLTAV | VLTQLSQPEP | | | | |
| | | WGFVHRYQR | EPPTVSDSKV | | | | |
| | | ELQDLLSDGE | QYLYLSSMRV | | | | |
| | | GQFEFKDFAF | FDAAAMSCPY | | | | |
| | | FIRDESVQTSA | LSGAPSLTAV | | | | |
| | | KLTEPSVIAKA | WGFVHRYQR | | | | |
| | | RSISQVKRTTII | ELQDLLSDGE | | | | |
| | | REDCSDLIFDI | GQFEFKDFAF | | | | |
| | | VIAIESDQRIS | FIRDESVQTSA | | | | |
| | | DYQSQFKAAL | KLTEPSVIAKA | | | | |
| | | PTNFAGGALF | RSISQVKRTTII | | | | |
| | | QPEINSGINW | REDCSDLIFDI | | | | |
| | | LRTFVSKSELF | VIAIESDQRIS | | | | |
| | | QAVKGLPGYG | DYQSQFKAAL | | | | |
| | | TWLSPDSFQP | PTNFAGGALF | | | | |
| | | QNLAELQECL | QPEINSGINW | | | | |
| | | TIDSSLIPVSN | LRTFVSKSELF | | | | |
| | | GFHFLGSPQE | QAVKGLPGYG | | | | |
| | | RKGALTKLHC | TWLSPDSFQP | | | | |
| | | YAENNIALAK | QNLAELQECL | | | | |
| | | RTNPIEVRFA | TIDSSLIPVSN | | | | |
| | | GSDHFFEQVF | GFHFLGSPQE | | | | |
| | | WSLEVTEQTIL | RKGALTKLHC | | | | |
| | | IKNKRI (SEQ | YAENNIALAK | | | | |
| | | ID NO: 289) | RTNPIEVRFA | | | | |
| | | | GSDHFFEQVF | | | | |
| | | | WSLEVTEQTIL | | | | |
| | | | IKNKRI (SEQ | | | | |
| | | | ID NO: 290) | | | | |
| 7 | Tn7015 | MVDKLKFHEL | MPKKKRKVGS | MELCNVLKY | MPKKKRKV | MHRYYFMV | MPKKKRKV |
| | | LDIDDISERNI | GDYKDDDDK | DRSLYPGKA | GSGELCNV | RFLPEQANL | GSGHRYYF |
| | | ALRRAFTGYT | DYKDDDDKD | VFFYKTAESD | LKYDRSLYP | ALLMGRCISI | MVRFLPEQ |
| | | VPMDVTGNE | YKDDDDKGS | FVPLEAEINRI | GKAVFFYKT | MHGFICKHD | ANLALLMG |
| | | ASALTILLNLTY | GVDKLKFHEL | RGQKAGFTE | AESDFVPLE | IQGLGVSFPA | RCISIMHGF |
| | | PRKRVDDLLD | LDIDDISERN | AFTPQFKSK | AEINRIRGQ | WSDASIGN | ICKHDIQGL |
| | | KRLAKQTLNT | ALRRAFTGYT | NLAPQDLAH | KAGFTEAFT | MIAFVHTDI | GVSFPAWS |
| | | DAHLDASIDE | VPMDVTGNE | CNPLILEECY | PQFKSKNL | AALNELKLQ | DASIGNMI |
| | | VQWLHTHNL | ASALTILLNLTY | VPPNVEYIYC | APQDLAHC | GYFQDMQE | AFVHTDIAA |
| | | KYPDIRVSKQ | PRKRVDDLLD | RFSLRVQAN | NPLILEECY | CGVFKVDNV | LNELKLQGY |
| | | RLITASPLSHS | KRLAKQTLNT | SLKPAGCSEP | VPPNVEYIY | EAVPDDCVE | FQDMQEC |
| | | HILSSANCISTL | DAHLDASIDE | TVFALLEEFA | CRFSLRVQ | VRFKRNQGI | GVFKVDNV |
| | | GWSHDSAKV | VQWLHTHNL | AIFKACGGYK | ANSLKPAG | AKMFVGEA | EAVPDDCV |
| | | NLAKLFSCHF | KYPDIRVSKQ | ELATRYCKN | CSEPTVFAL | RRRLKRLEKR | EVRFKRNQ |
| | | NWQDRVCCL | RLITASPLSHS | VLLGTWLW | LEEFAAIFK | ALARGEVFN | GIAKMFVG |
| | | ATLLSDPPKI | HILSSANCISTL | RNQNTGNS | ACGGYKEL | PNKNDEPRE | EARRRLKRL |
| | | WKEAFQALG | GWSHDSAKV | QIDIKTSAGN | ATRYCKNV | LDCFHCIAIG | EKRALARG |
| | | MLVKDFMNL | NLAKLFSCHF | CYQIANTRQ | LLGTWLWR | STSTEQDFLL | EVFNPNKN |
| | | CGRIKASLPSY | NWQDRVCCL | LAWDSRWP | NQNTGNS | HVQKEIVQK | DEPRELDCF |
| | | ESPSRVDKYSI | ATLLSDPPKI | ADAQQVLEE | QIDIKTSAG | YEEPEFNQY | HCIAIGSTST |
| | | QVRLPYRDGY | WKEAFQALG | LSDEVHQAL | NCYQIANT | GLATNKLLR | EQDFLLHV |
| | | LAITPVVSHAL | MLVKDFMNL | TDPTVFWH | RQLAWDSR | GTVPEFSEF | QKEIVQKYE |
| | | QAEIQQAAM | CGRIKASLPSY | ANITAKIETA | WPADAQQ | (SEQ ID | EPEFNQYG |
| | | AKQCRYTNFE | ESPSRVDKYSI | FCQEIYPSQS | VLEELSDEV | NO: 299) | LATNKLLRG |
| | | FTRPAAVSELS | QVRLPYRDGY | FGEKAAQGE | HQALTDPT | | TVPEFSEF |
| | | ASLGGNVKAL | LAITPVVSHAL | ASKQFAKVK | VFWHANIT | | (SEQ ID |
| | | NYPPRIGNAV | QAEIQQAAM | CVDGRYAVS | AKIETAFCQ | | NO: 300) |
| | | HGLSDSWLLK | AKQCRYTNFE | FNSVKIGAAL | EIYPSQSFG | | |
| | | FQAGQTVLN | FTRPAAVSELS | QLIDDWWD | EKAAQGEA | | |
| | | QGALSQPRFK | ASLGGNVKAL | VDDSKRLRIH | SKQFAKVK | | |
| | | RALEGLLSNG | NYPPRIGNAV | EYGADKELG | CVDGRYAV | | |
| | | FELALKQRRL | HGLSDSWLLK | VARRAPESK | SFNSVKIGA | | |
| | | HKVASMRQIR | FQAGQTVLN | QSFYSLFINT | ALQLIDDW | | |
| | | ATLTEWLSPLL | QGALSQPRFK | ELYLAELNQ | WDVDDSK | | |
| | | EWRLEVEENK | RALEGLLSNG | QLAEDEYSIS | RLRIHEYGA | | |
| | | NNVSELACIH | FELALKQRRL | PNIYYLFAVLI | DKELGVAR | | |
| | | GSFEYQFLTA | HKVASMRQIR | KGGMFQKK | RAPESKQSF | | |
| | | QKENLVGLLN | ATLTEWLSPLL | AEAKSKSKAE | YSLFINTELY | | |
| | | PMFSLLNTILS | EWRLEVEENK | TSTAKITPAK | LAELNQQL | | |
| | | NSNTLQKYAF | NNVSELACIH | A (SEQ ID | AEDEYSISP | | |
| | | HQRLMRPLKC | GSFEYQFLTA | NO: 297) | NIYYLFAVLI | | |
| | | SLKWLLDNLS | QKENLVGLLN | | KGGMFQK | | |
| | | KESNAIDSDE | PMFSLLNTILS | | KAEAKSKSK | | |
| | | DNQQRYLYLK | NSNTLQKYAF | | AETSTAKIT | | |
| | | GIRVFDAQAL | HQRLMRPLKC | | PAKA (SEQ | | |
| | | SNPYCAGLPSL | SLKWLLDNLS | | ID NO: 298) | | |
| | | TAVWGMVH | KESNAIDSDE | | | | |
| | | NYQRRLNKRL | DNQQRYLYLK | | | | |
| | | GTQLRLTSFS | GIRVFDAQAL | | | | |
| | | WFIRQYSSVA | SNPYCAGLPSL | | | | |
| | | GKKLPEYGM | TAVWGMVH | | | | |
| | | QGQKENQFR | NYQRRLNKRL | | | | |
| | | RAGIVDNKHC | GTQLRLTSFS | | | | |
| | | DLVFDLVVHI | WFIRQYSSVA | | | | |
| | | DGYEEDLDAI | GKKLPEYGM | | | | |
| | | DNSTDAIKAS | QGQKENQFR | | | | |
| | | FPATFAGGV | RAGIVDNKHC | | | | |
| | | MHPPEIGSVD | DLVFDLVVHI | | | | |
| | | EWCELYPSET | DGYEEDLDAI | | | | |
| | | SLYSKLRRLPA | DNSTDAIKAS | | | | |
| | | SGKWVMPTR | FPATFAGGV | | | | |
| | | YQMDSLDGLL | MHPPEIGSVD | | | | |
| | | QLLKLNVALC | EWCELYPSET | | | | |
| | | PVMSGYLML | SLYSKLRRLPA | | | | |
| | | GPPESRKNSL | SGKWVMPTR | | | | |
| | | EPLHCYAEPAI | YQMDSLDGLL | | | | |
| | | GVVECATAIDI | QLLKLNVALC | | | | |
| | | RLQGMSNFF | PVMSGYLML | | | | |
| | | RRAFWMLDI | GPPESRKNSL | | | | |
| | | KETSMLMKRI | EPLHCYAEPAI | | | | |
| | | (SEQ ID | GVVECATAIDI | | | | |
| | | NO: 295) | RLQGMSNFF | | | | |
| | | | RRAFWMLDI | | | | |
| | | | KETSMLMKRI | | | | |
| | | | (SEQ ID | | | | |
| | | | NO: 296) | | | | |
| 8 | Tn7016 | MHLKELLEITD | MPKKKRKVGS | MELCNILKY | MPKKKRKV | MQRYYFTVH | MPKKKRKV |
| | | TTERDRSLRR | GDYKDDDDK | DRSLYPGKA | GSGELCNIL | FLPKQANLA | GSGQRYYF |
| | | AFSPYTAMIDI | DYKDDDDKD | VFFYKTADS | KYDRSLYPG | LLTGRCISIM | TVHFLPKQ |
| | | TGSEAVALIILL | YKDDDDKGS | DFVPLEADIN | KAVFFYKTA | HGFILKHNIE | ANLALLTGR |
| | | NLTYRKNQVD | GHLKELLEITD | KIRGPKSGFT | DSDFVPLEA | GMGVTFPA | CISIMHGFIL |
| | | DLLDKKLAKQ | TTERDRSLRR | EAFTPQFSPK | DINKIRGPK | WSDSSIGNEI | KHNIEGMG |
| | | ALKSEDHINKC | AFSPYTAMIDI | NISPQDLTH | SGFTEAFTP | AFVYTDKEIL | VTFPAWSD |
| | | IKEIAWFHTH | TGSEAVALIILL | NNILTLEECY | QFSPKNISP | NTLKDQAYF | SSIGNEIAF |
| | | NLKYPDIRVSK | NLTYRKNQVD | VPPNVEHIFC | QDLTHNNI | VDMQDCGF | VYTDKEILN |
| | | QNLAVEPPTL | DLLDKKLAKQ | RFSLRVQAN | LTLEECYVP | FKVSQVLAV | TLKDQAYF |
| | | HSYVLSSANY | ALKSEDHINKC | SLVPSGCSDP | PNVEHIFCR | PDSCEEVRFI | VDMQDCG |
| | | PKAYGWSHN | IKEIAWFHTH | EVFSLLKELA | FSLRVQAN | RNQAVAKIF | FFKVSQVLA |
| | | SAKVNFAKLF | NLKYPDIRVSK | ETFKECGGY | SLVPSGCSD | TGESRRRLKR | VPDSCEEV |
| | | VSYFKWQNQ | QNLAVEPPTL | KELAVRYCR | PEVFSLLKEL | LQKRALARG | RFIRNQAV |
| | | VSWLAQVLAT | HSYVLSSANY | NILIGTWLW | AETFKECG | EDFNPKKIEA | AKIFTGESR |
| | | NSDNWKSAF | PKAYGWSHN | RNQNTGNT | GYKELAVRY | PREIDIFHRV | RRLKRLQKR |
| | | TSLGLSVKAFK | SAKVNFAKLF | QIEIKTSKGS | CRNILIGTW | AMTSKSSQE | ALARGEDF |
| | | SLCVTVKNSLP | VSYFKWQNQ | CYLIDNTRKL | LWRNQNT | DYILHIQKQD | NPKKIEAPR |
| | | EEAIPDSVDRY | VSWLAQVLAT | AWESKWAS | GNTQIEIKT | VDCQAEPYF | EIDIFHRVA |
| | | SRQIRMPYHD | NSDNWKSAF | DDLKVLEELS | SKGSCYLID | SNYGLASNE | MTSKSSQE |
| | | GYLAVTPVISH | TSLGLSVKAFK | NEIESALTDP | NTRKLAWE | KFKGTVPDLS | DYILHIQKQ |
| | | VVQSKIQQAA | SLCVTVKNSLP | NVFWSADIT | SKWASDDL | PSIDRN (SEQ | DVDCQAEP |
| | | IDKRARFSNV | EEAIPDSVDRY | AKIEASFCQE | KVLEELSNEI | ID NO: 305) | YFSNYGLAS |
| | | EFTRPAAVSM | SRQIRMPYHD | IYPSQILNDK | ESALTDPN | | NEKFKGTV |
| | | LAASLGGVIN | GYLAVTPVISH | VKQGEASKQ | VFWSADIT | | PDLSPSIDR |
| | | VLNYPPYIRSK | VVQSKIQQAA | FVKAKCADG | AKIEASFCQ | | N (SEQ ID |
| | | YHGLSNSRAF | IDKRARFSNV | RYAVSFNSV | EIYPSQILN | | NO: 306) |
| | | KLNNGQTVF | EFTRPAAVSM | KIGAALQSID | DKVKQGEA | | |
| | | NVEALLKPELI | LAASLGGVIN | DWWDEDAS | SKQFVKAK | | |
| | | KALEGIIFSNN | VLNYPPYIRSK | KRLRVHEFG | CADGRYAV | | |
| | | ALALKQRRQQ | YHGLSNSRAF | ADKEIGVAR | SFNSVKIGA | | |
| | | KVKNIKELRNT | KLNNGQTVF | RPPDSEQNF | ALQSIDDW | | |
| | | LLEWFSPVFE | NVEALLKPELI | YSIFKNTEWY | WDEDASKR | | |
| | | WRLDAIENGY | KALEGIIFSNN | LSALKNCITN | LRVHEFGA | | |
| | | DLEQLESASER | ALALKQRRQQ | KNEKIDPAIY | DKEIGVARR | | |
| | | LEYKILSLPDN | KVKNIKELRNT | YLFSVLIKGG | PPDSEQNF | | |
| | | ELPSLTIPLFRL | LLEWFSPVFE | MFQKKAEAK | YSIFKNTEW | | |
| | | LNEMLGGVS | WRLDAIENGY | K (SEQ ID | YLSALKNCI | | |
| | | MTQRYAFHP | DLEQLESASER | NO: 303 | TNKNEKIDP | | |
| | | KLMSPLKAAL | LEYKILSLPDN | | AIYYLFSVLI | | |
| | | QWLLVNLTD | ELPSLTIPLFRL | | KGGMFQK | | |
| | | QKHVLIEEDD | LNEMLGGVS | | KAEAKK | | |
| | | EHYRYLHLSGI | MTQRYAFHP | | (SEQ ID | | |
| | | RVFDAQALSN | KLMSPLKAAL | | NO: 304) | | |
| | | PYCSGIPSLTA | QWLLVNLTD | | | | |
| | | VWGMIHSYQ | QKHVLIEEDD | | | | |
| | | RKLNEALGTN | EHYRYLHLSGI | | | | |
| | | VRFTSFSWFIR | RVFDAQALSN | | | | |
| | | NYSAVAGKKL | PYCSGIPSLTA | | | | |
| | | PELSLQGAQQ | VWGMIHSYQ | | | | |
| | | SRLKRPGIIDG | RKLNEALGTN | | | | |
| | | KYCDLVEDLII | VRFTSFSWFIR | | | | |
| | | HIDGYEDDLQ | NYSAVAGKKL | | | | |
| | | AVDSKPDILKA | PELSLQGAQQ | | | | |
| | | HFPSNFAGGV | SRLKRPGIIDG | | | | |
| | | MHQPELNSNI | KYCDLVEDLII | | | | |
| | | NWCCLYSNE | HIDGYEDDLQ | | | | |
| | | NQLFEKLRRLP | AVDSKPDILKA | | | | |
| | | LSGCWVMPT | HFPSNFAGGV | | | | |
| | | EHKIQDLDELL | MHQPELNSNI | | | | |
| | | LLLNSDSKLSP | NWCCLYSNE | | | | |
| | | SMMGYMLLT | NQLFEKLRRLP | | | | |
| | | EPMARVGSLE | LSGCWVMPT | | | | |
| | | RLHCYAEPAIG | EHKIQDLDELL | | | | |
| | | VVKYEAATSV | LLLNSDSKLSP | | | | |
| | | RLKGIGNYFN | SMMGYMLLT | | | | |
| | | SAFWMLDAQ | EPMARVGSLE | | | | |
| | | EKFMLMKKV | RLHCYAEPAIG | | | | |
| | | (SEQ ID | VVKYEAATSV | | | | |
| | | NO: 301) | RLKGIGNYFN | | | | |
| | | | SAFWMLDAQ | | | | |
| | | | EKFMLMKKV | | | | |
| | | | (SEQ ID | | | | |
| | | | NO: 302) | | | | |
| 10 | V.para_UCM- | MIKLGDVLAIE | MPKKKRKVGS | MELCSQLNY | MPKKKRKV | MSKRYYFSIR | MPKKKRKV |
| | V493 | EDEVKQATLK | GDYKDDDDK | VRSLSAGKA | GSGELCSQL | YIPLHADFGL | GSGSKRYYF |
| | AHI99014 | KVFMPYSENI | DYKDDDDKD | CFYYLTPSGD | NYVRSLSA | LAGRCIQQM | SIRYIPLHAD |
| | | DIDGREREALT | YKDDDDKGS | MCPLSIDKTR | GKACFYYLT | HMFIVNNP | FGLLAGRCI |
| | | VLINLSSHHKG | GIKLGDVLAIE | LRAPKGGYS | PSGDMCPL | QVKNKVGV | QQMHMFI |
| | | SKCTDWLDID | EDEVKQATLK | EAYRGSQFH | SIDKTRLRA | CFPRWNVT | VNNPQVK |
| | | RAKSYLSQEA | KVFMPYSENI | QKNVAPQDL | PKGGYSEA | NIGDTIAFV | NKVGVCFP |
| | | NVDLSLAEIK | DIDGREREALT | AYANPQFIEE | YRGSQFHQ | MDDKEMLS | RWNVTNIG |
| | | WFHTHNLKY | VLINLSSHHKG | CYVPPSTDEI | KNVAPQDL | GLSFQPYFS | DTIAFVMD |
| | | PDCRVSAQRII | SKCTDWLDID | VCEFSLRVKA | AYANPQFIE | MMVKEGVF | DKEMLSGL |
| | | AEPLPAEDAFI | RAKSYLSQEA | NSLHPEVCN | ECYVPPSTD | EVSRVCEVP | SFQPYFSM |
| | | SSSGLPPSLG | NVDLSLAEIK | DDSVREQLA | EIVCEFSLR | VDSPEVRFV | MVKEGVFE |
| | | WAHNSASYR | WFHTHNLKY | LLAATYKNLN | VKANSLHP | RNQIIGKSFV | VSRVCEVP |
| | | HTIWLLSSFC | PDCRVSAQRII | GYQELAYRY | EVCNDDSV | ASKQRRMK | VDSPEVRF |
| | | WQSRTFSIVS | AEPLPAEDAFI | AKNILLGTW | REQLALLAA | RSMLRADLS | VRNQIIGKS |
| | | LIQQQNPVW | SSSGLPPSLG | LWRNRECR | TYKNLNGY | ATEHTPIAKE | FVASKQRR |
| | | LDLLQEFGLSV | WAHNSASYR | GVAIEVTTSD | QELAYRYA | ERVVDHFHR | MKRSMLR |
| | | KSLNLISEEIEL | HTIWLLSSFC | GEIILISDATR | KNILLGTWL | VPISSASSGQ | ADLSATEHT |
| | | QLLSTAFPTEV | WQSRTFSIVS | LSWYGHWD | WRNRECR | EYLLHIQKEF | PIAKEERVV |
| | | NTYSKQLRFP | LIQQQNPVW | EKSTESLERL | GVAIEVTTS | VESREQANF | DHFHRVPIS |
| | | WNGDYLSVT | LDLLQEFGLSV | TSYLSRALSD | DGEIILISDA | NSYGLATNQ | SASSGQEYL |
| | | PVVSHAMQS | KSLNLISEEIEL | NAQYFYMD | TRLSWYGH | EKRGTVPDL | LHIQKEFVE |
| | | ELEHRQRSED | QLLSTAFPTEV | VKAVLAVGR | WDEKSTES | SI (SEQ ID | SREQANFN |
| | | SHLKFVTMLL | NTYSKQLRFP | GDEVYPSQE | LERLTSYLSR | NO: 311) | SYGLATNQ |
| | | PNSASIGNLC | WNGDYLSVT | FLDDKQEGV | ALSDNAQY | | EKRGTVPD |
| | | GSVGGYMKV | PVVSHAMQS | PTKQLAKVR | FYMDVKAV | | LSI (SEQ ID |
| | | LNYPLDISPKV | ELEHRQRSED | LDDGRETAA | LAVGRGDE | | NO: 312) |
| | | NRASSEQTLG | SHLKFVTMLL | FHAQKIGAA | VYPSQEFLD | | |
| | | ASRQRNGRCF | PNSASIGNLC | LQSIDDWW | DKQEGVPT | | |
| | | DDYQITNIRIC | GSVGGYMKV | HEEADKPLR | KQLAKVRL | | |
| | | EILNRLVGAEP | LNYPLDISPKV | VNEYGADRE | DDGRETAA | | |
| | | LKTHKQRVKA | NRASSEQTLG | YVIARRHTQS | FHAQKIGA | | |
| | | RKDQSKILRK | ASRQRNGRCF | GNDFYQLIR | ALQSIDDW | | |
| | | QIALWMLPLI | DDYQITNIRIC | RTEAWTEE | WHEEADKP | | |
| | | ELRDRMVND | EILNRLVGAEP | MEKLKSIPN | LRVNEYGA | | |
| | | ERERTMHGD | LKTHKQRVKA | DVHFIMSVLI | DREYVIARR | | |
| | | QLIHDFLFLPE | RKDQSKILRK | KGGLFNSSKS | HTQSGNDF | | |
| | | RELSSLATSLN | QIALWMLPLI | TAK (SEQ ID | YQLIRRTEA | | |
| | | QKLHLVLQGN | ELRDRMVND | NO: 309) | WTEEMEKL | | |
| | | KFTRKFAYHP | ERERTMHGD | | KSIPNDVHF | | |
| | | RLMQLIKAQI | QLIHDFLFLPE | | IMSVLIKGG | | |
| | | VWILDVLSKP | RELSSLATSLN | | LFNSSKSTA | | |
| | | QQQEGGCGA | QKLHLVLQGN | | K (SEQ ID | | |
| | | EEQYIYLSSLR | KFTRKFAYHP | | NO: 310) | | |
| | | VQDALAVSSP | RLMQLIKAQI | | | | |
| | | YLCGVPSLTAI | VWILDVLSKP | | | | |
| | | WGFVHQYQR | QQQEGGCGA | | | | |
| | | DFNTLTNGDA | EEQYIYLSSLR | | | | |
| | | FYDFTGFAFY | VQDALAVSSP | | | | |
| | | VRSQNIIATAK | YLCGVPSLTAI | | | | |
| | | LTEPCSLAKAR | WGFVHQYQR | | | | |
| | | TLSNAKRSTIR | DFNTLTNGDA | | | | |
| | | GDRLTDLEIDL | FYDFTGFAFY | | | | |
| | | VIRVQSRGRLS | VRSQNIIATAK | | | | |
| | | DCSSELKNALP | LTEPCSLAKAR | | | | |
| | | VSFAGGSVFQ | TLSNAKRSTIR | | | | |
| | | PRISSKIDWLR | GDRLTDLEIDL | | | | |
| | | TFCSRSSLLHIL | VIRVQSRGRLS | | | | |
| | | KGLPAYGSWL | DCSSELKNALP | | | | |
| | | YPSERQPESF | VSFAGGSVFQ | | | | |
| | | DELELMLLEN | PRISSKIDWLR | | | | |
| | | ENYLPVSNGY | TFCSRSSLLHIL | | | | |
| | | HLLEVPTQRK | KGLPAYGSWL | | | | |
| | | NSLTDLHAYV | YPSERQPESF | | | | |
| | | ENTLSVANQV | DELELMLLEN | | | | |
| | | NPIEMRFSGR | ENYLPVSNGY | | | | |
| | | APFFEQAFWS | HLLEVPTQRK | | | | |
| | | LECRPTTILIKK | NSLTDLHAYV | | | | |
| | | L (SEQ ID | ENTLSVANQV | | | | |
| | | NO: 307) | NPIEMRFSGR | | | | |
| | | | APFFEQAFWS | | | | |
| | | | LECRPTTILIKK | | | | |
| | | | L (SEQ ID | | | | |
| | | | NO: 308) | | | | |
| 11 | Aliiglaciecola | MASNEITSLL | MPKKKRKVGS | MRLPNRLSY | MPKKKRKV | MASRYYRKIT | MPKKKRKV |
| | sp. M165 | NIENHTDRNV | GDYKDDDDK | QRSISPGIAV | GSGRLPNR | FIPADSNHN | GSGASRYY |
| | | AWKKALSPIT | DYKDDDDKD | FYSVDEQGN | LSYQRSISP | FLIGKCLKVL | RKITFIPADS |
| | | PPLDVTGNEK | YKDDDDKGS | QKPLEINTVK | GIAVFYSVD | HGVNCRHRL | NHNFLIGKC |
| | | LACVVLANLT | GASNEITSLLN | ILGQKGGPS | EQGNQKPL | NSIGVTFPD | LKVLHGVN |
| | | WKLSLINNVF | IENHTDRNVA | EAFANDMSL | EINTVKILG | WSDESPGNS | CRHRLNSIG |
| | | DSNDARAKLR | WKKALSPITP | KKGVDNKKL | QKGGPSEA | IAFVSVDSAC | VTFPDWSD |
| | | DKNWIQRCIK | PLDVTGNEKL | AEGNPHTID | FANDMSLK | IDLLIDQHYY | ESPGNSIAF |
| | | TFRYRHTHNL | ACVVLANLT | YCYAPADAK | KGVDNKKL | QQMQDLEY | VSVDSACID |
| | | KYPDYRAKGA | WKLSLINNVF | HTLCKFSLNV | AEGNPHTI | FEISALKPVP | LLIDQHYYQ |
| | | IRLSPIGVIPKG | DSNDARAKLR | DASSIEPRAC | DYCYAPAD | ENGSEEIMF | QMQDLEYF |
| | | CFSSSKLISSRL | DKNWIQRCIK | NDDGVRSLL | AKHTLCKFS | SRNQAVDEL | EISALKPVP |
| | | GWSQNSADI | TFRYRHTHNL | TNFAAEYRKL | LNVDASSIE | TPAGVRRKL | ENGSEEIMF |
| | | NYATFLCADF | KYPDYRAKGA | GGYRYLAER | PRACNDDG | RRCARRAKQ | SRNQAVDE |
| | | VWQGELLTLG | IRLSPIGVIPKG | YLNNVLSGN | VRSLLTNFA | RGENYNAAY | LTPAGVRR |
| | | EAIIGENISFTK | CFSSSKLISSRL | WLWRNQRT | AEYRKLGG | LSSSEKVFPH | KLRRCARR |
| | | SLIESGMFKK | GWSQNSADI | LDTTIKIQSS | YRYLAERYL | FHKIPMNSK | AKQRGENY |
| | | DLKLIRNELSQ | NYATFLCADF | GGLQCSIKG | NNVLSGN | SSDRNFSLNI | NAAYLSSSE |
| | | IPINQTESEYLS | VWQGELLTLG | VNRKRFEPN | WLWRNQR | QLEMAQNV | KVFPHFHKI |
| | | HQLTNLRFPK | EAIIGENISFTK | WIDEITEFDG | TLDTTIKIQS | TYGNYTSYG | PMNSKSSD |
| | | HSDGYVCLTP | SLIESGMFKK | LVNEFENAL | SGGLQCSIK | LSNKSSRKAS | RNFSLNIQL |
| | | VPSHIVQVAI | DLKLIRNELSQ | VDPKKYLFLE | GVNRKRFE | VPKNLD | EMAQNVT |
| | | HSWSVSNFR | IPINQTESEYLS | VTAELSLPLA | PNWIDEITE | (SEQ ID | YGNYTSYGL |
| | | QSETMYCPRS | HQLTNLRFPK | SEIYPSQAFV | FDGLVNEF | NO: 317) | SNKSSRKAS |
| | | SSVGSLPACV | HSDGYVCLTP | EQANKLERS | ENALVDPK | | VPKNLD |
| | | GGKIKVLKSLP | VPSHIVQVAI | RTYQNTIVE | KYLFLEVTA | | (SEQ ID |
| | | KGLNSKHTKD | HSWSVSNFR | GKRTAIIGAY | ELSLPLASEI | | NO: 318) |
| | | TQKSSWLTAE | QSETMYCPRS | KIGAAIASID | YPSQAFVE | | |
| | | NLAILHSLSSS | SSVGSLPACV | DWFEGADIP | QANKLERS | | |
| | | RDWLLPENKK | GGKIKVLKSLP | VRVGSFAVD | RTYQNTIVE | | |
| | | KKRYKELVAKL | KGLNSKHTKD | RDRATVYRH | GKRTAIIGA | | |
| | | GAMLVRWM | TQKSSWLTAE | PESKKDFYTL | YKIGAAIASI | | |
| | | SFNRKSLEQLL | NLAILHSLSSS | LSGLEQLNSR | DDWFEGA | | |
| | | ESEFPSKQITQ | RDWLLPENKK | LKSKKKMKS | DIPVRVGSF | | |
| | | LFHADLSRLKS | KKRYKELVAKL | SELNDAHFIA | AVDRDRAT | | |
| | | TDDIAYNPTFI | GAMLVRWM | ANLVKGGLF | VYRHPESKK | | |
| | | KIVEQEFKIILE | SFNRKSLEQLL | SLGSK (SEQ | DFYTLLSGL | | |
| | | NEKEDYPLVIP | ESEFPSKQITQ | ID NO: 315) | EQLNSRLKS | | |
| | | QQKHTHLVLP | LFHADLSRLKS | | KKKMKSSEL | | |
| | | GLRVSNANAE | TDDIAYNPTFI | | NDAHFIAA | | |
| | | SCAYLVGLPS | KIVEQEFKIILE | | NLVKGGLFS | | |
| | | MIGIFGFIHNL | NEKEDYPLVIP | | LGSK (SEQ | | |
| | | QRQLDSRFGL | QQKHTHLVLP | | ID NO: 316) | | |
| | | SAGFEQFAIC | GLRVSNANAE | | | | |
| | | MHEYSFHKR | SCAYLVGLPS | | | | |
| | | GLTKEQVQIS | MIGIFGFIHNL | | | | |
| | | KKQLRSPAIID | QRQLDSRFGL | | | | |
| | | SRQCDFALSL | SAGFEQFAIC | | | | |
| | | VIKTSAILQRE | MHEYSFHKR | | | | |
| | | EVLAALPQKIC | GLTKEQVQIS | | | | |
| | | GGAVHIPLSEL | KKQLRSPAIID | | | | |
| | | EGINTHHSFES | SRQCDFALSL | | | | |
| | | AVNAIPVKNG | VIKTSAILQRE | | | | |
| | | KWITPSFNSLS | EVLAALPQKIC | | | | |
| | | TTNFIDFLDKT | GGAVHIPLSEL | | | | |
| | | SVSYNLNIACV | EGINTHHSFES | | | | |
| | | GYHYLETPFKK | AVNAIPVKNG | | | | |
| | | NSASDDPVHA | KWITPSFNSLS | | | | |
| | | FAEPILAGVQL | TTNFIDFLDKT | | | | |
| | | NCIASFGNIER | SVSYNLNIACV | | | | |
| | | FFWHYSETST | GYHYLETPFKK | | | | |
| | | SLYLGSKI | NSASDDPVHA | | | | |
| | | (SEQ ID | FAEPILAGVQL | | | | |
| | | NO: 313) | NCIASFGNIER | | | | |
| | | | FFWHYSETST | | | | |
| | | | SLYLGSKI | | | | |
| | | | (SEQ ID | | | | |
| | | | NO: 314) | | | | |
| 12 | Oceanospirillum | MLKDLLEKKE | MPKKKRKVGS | MNLPNQLTY | MPKKKRKV | MKWHYFIIR | MPKKKRKV |
| | linum | GTRAEFNHKV | GDYKDDDDK | KRSLHPGPA | GSGNLPNQ | YIPSDADEFL | GSGKWHYF |
| | ATCC | KRCFEPYTPLI | DYKDDDDKD | VFFYEDAEEK | LTYKRSLHP | LAGRCILALH | IIRYIPSDAD |
| | 11336 | EADGAELECVI | YKDDDDKGS | QHPLTIERTK | GPAVFFYE | HFLYRNKAN | EFLLAGRCIL |
| | | ILANLASRAAE | GLKDLLEKKE | IRGSKSGFAE | DAEEKQHP | SIGIHFPDWS | ALHHFLYR |
| | | TLDDRASAKS | GTRAEFNHKV | AYQVKKDKA | LTIERTKIRG | DRSVGKRIAF | NKANSIGIH |
| | | SLTTDNFWKK | KRCFEPYTPLI | AESGINISLK | SKSGFAEAY | MSENEDLLT | FPDWSDRS |
| | | VLQSAQQLHT | EADGAELECVI | PDATTQKLSS | QVKKDKAA | WFKKERYFL | VGKRIAFM |
| | | HNLKFPDARV | ILANLASRAAE | GNPHTIDTC | ESGINISLKP | TMAENDLFE | SENEDLLT |
| | | HYKNRIRVINP | TLDDRASAKS | YLPPEAETLIC | DATTQKLSS | MTEIVQTSLT | WFKKERYF |
| | | QDQFPVLGW | SLTTDNFWKK | KFSLRIAANS | GNPHTIDT | DKKGVAFVR | LTMAENDL |
| | | SGNSSDYNFA | VLQSAQQLHT | LKPDTCSDA | CYLPPEAET | NQKAGKLTS | FEMTEIVQT |
| | | RFLNSAFQW | HNLKFPDARV | ECWNSLTNF | LICKFSLRIA | ASKARRIRRA | SLTDKKGV |
| | | QNERHTLLTV | HYKNRIRVINP | TALYKKAGG | ANSLKPDT | KRRAEARGE | AFVRNQKA |
| | | LLDDLPAWRN | QDQFPVLGW | YFELAERYAK | CSDAECWN | VYKSRNQES | GKLTSASKA |
| | | AFSRLGVFKA | SGNSSDYNFA | NILSGAWLW | SLTNFTALY | DRELDHFHSI | RRIRRAKRR |
| | | QWHQLRQQL | RFLNSAFQW | RNRDTAAFEI | KKAGGYFEL | HMESTSTGK | AEARGEVY |
| | | KQIFQTSTFPD | QNERHTLLTV | TVETSEGNT | AERYAKNIL | AFTLFVGKVE | KSRNQESD |
| | | TVDIYSPQLRL | LLDDLPAWRN | YTLPNAHLQ | SGAWLWR | EPGTGLSQK | RELDHFHSI |
| | | PWRGRHLIAI | AFSRLGVFKA | FPDIPWKKD | NRDTAAFEI | EFNSYGLSSQ | HMESTSTG |
| | | TPVVNHTLQL | QWHQLRQQL | TAKILKGLAT | TVETSEGNT | NQQMVLLPI | KAFTLFVGK |
| | | KIQSSAKELPSI | KQIFQTSTFPD | EIETALASPR | YTLPNAHL | IS (SEQ ID | VEEPGTGLS |
| | | KISYPRPSAIG | TVDIYSPQLRL | YYWSAEITA | QFPDIPWK | NO: 323) | QKEFNSYG |
| | | QLCGALGGNL | PWRGRHLIAI | RLKPGFCAEI | KDTAKILKG | | LSSQNQQ |
| | | RYLHYHPIPKG | TPVVNHTLQL | FPSQCFTDPS | LATEIETAL | | MVLLPIIS |
| | | LIGFQQQLSV | KIQSSAKELPSI | DSDASKVLA | ASPRYYWS | | (SEQ ID |
| | | DRESLLSQRSL | KISYPRPSAIG | TINYQGAKT | AEITARLKP | | NO: 324) |
| | | SGKHPESVYK | QLCGALGGNL | ACMTADKV | GFCAEIFPS | | |
| | | SLIDRRINASL | RYLHYHPIPKG | NAAIQRVDN | QCFTDPSD | | |
| | | RLARLARRDA | LIGFQQQLSV | WYSDDPNA | SDASKVLAT | | |
| | | LRQFDLILEN | DRESLLSQRSL | SPLRVNEYGS | INYQGAKT | | |
| | | WLKALMDVR | SGKHPESVYK | DSHRNIACR | ACMTADKV | | |
| | | QYFLETGCLH | SLIDRRINASL | HPSTQLDFY | NAAIQRVD | | |
| | | YKNLNRVEES | RLARLARRDA | TLLQGIDEQI | NWYSDDP | | |
| | | FVRDEASSND | LRQFDLILEN | SVLEKAKSLK | NASPLRVN | | |
| | | LRKYLNTSFHK | WLKALMDVR | DIPASTHYITS | EYGSDSHR | | |
| | | SLRLNPYTQD | QYFLETGCLH | VLTKGGMF | NIACRHPST | | |
| | | FAYHPGLTAT | YKNLNRVEES | QGGKAK* | QLDFYTLLQ | | |
| | | LNQRLKQLLH | FVRDEASSND | (SEQ ID | GIDEQISVL | | |
| | | QENAPSAAEE | LRKYLNTSFHK | NO: 321) | EKAKSLKDI | | |
| | | LPEMGYASLH | SLRLNPYTQD | | PASTHYITS | | |
| | | NVSVTDGNAL | FAYHPGLTAT | | VLTKGGMF | | |
| | | NNPYCAGMP | LNQRLKQLLH | | QGGKAK* | | |
| | | SMTGLWGFC | QENAPSAAEE | | (SEQ ID | | |
| | | KNLEMQLKES | LPEMGYASLH | | NO: 322) | | |
| | | GFAVSVQRVA | NVSVTDGNAL | | | | |
| | | LMCHEFSANR | NNPYCAGMP | | | | |
| | | STLIPEPSRPSP | SMTGLWGFC | | | | |
| | | QKGSQTVKRS | KNLEMQLKES | | | | |
| | | GLLPQFTFSG | GFAVSVQRVA | | | | |
| | | QFSVVIEYRKS | LMCHEFSANR | | | | |
| | | AGRLSELTTD | STLIPEPSRPSP | | | | |
| | | DLRNHLPDRL | QKGSQTVKRS | | | | |
| | | WGGSLMLQE | GLLPQFTFSG | | | | |
| | | SANNHGIHLT | QFSVVIEYRKS | | | | |
| | | DEFDPLYRKLI | AGRLSELTTD | | | | |
| | | RQFRRGVWL | DLRNHLPDRL | | | | |
| | | VPDSSEVIEQ | WGGSLMLQE | | | | |
| | | NSLFDLLLEDK | SANNHGIHLT | | | | |
| | | KRAPLLTGFK | DEFDPLYRKLI | | | | |
| | | ALEEPKIREGA | RQFRRGVWL | | | | |
| | | LCGLHFYAEP | VPDSSEVIEQ | | | | |
| | | AIGICRRETMF | NSLFDLLLEDK | | | | |
| | | RLTKSPDYFLN | KRAPLLTGFK | | | | |
| | | KAFWGLTPAT | ALEEPKIREGA | | | | |
| | | NNDESIHLIRR | LCGLHFYAEP | | | | |
| | | V (SEQ ID | AIGICRRETMF | | | | |
| | | NO: 319) | RLTKSPDYFLN | | | | |
| | | | KAFWGLTPAT | | | | |
| | | | NNDESIHLIRR | | | | |
| | | | V (SEQ ID | | | | |
| | | | NO: 320) | | | | |
| 14 | V. | MQTLKELIEST | MPKKKRKVGS | MKLPTSLAY | MPKKKRKV | MNWYNKTI | MPKKKRKV |
| | anguillarum | PDDLTTVLKR | GDYKDDDDK | ERSIDPSDVC | GSGKLPTSL | TFLPERCDNE | GSGNWYN |
| | J360_ | AFRPLTPHIAI | DYKDDDDKD | FFVVWPDDK | AYERSIDPS | VLAAKCLSTL | KTITFLPERC |
| | AZS27374.1 | DGNELDALTIL | YKDDDDKGS | KTPLTYTSRT | DVCFFVVW | HAFNYKYDT | DNEVLAAK |
| | | VNLTDKTDDQ | GQTLKELIEST | LLGQMETAS | PDDKKTPLT | RSIGISFPGW | CLSTLHAFN |
| | | KDLLDRAKCK | PDDLTTVLKR | LAYDASGQPI | YTSRTLLGQ | CEDTVGKKL | YKYDTRSIGI |
| | | QKLRDEKWW | AFRPLTPHIAI | KSATAEALA | METASLAY | TFISTSKVELD | SFPGWCED |
| | | ASCLNCVNYR | DGNELDALTIL | QGNPHQVDI | DASGQPIKS | LLLKHQYFIQ | TVGKKLTFI |
| | | QSHNPKFPDI | VNLTDKTDDQ | CRVPFGASH | ATAEALAQ | MRKLSYFDIS | STSKVELDL |
| | | RSEGIIRTEAL | KDLLDRAKCK | VECCFSVSFS | GNPHQVDI | ATAQIPDGC | LLKHQYFIQ |
| | | GELPSFLLSSS | QKLRDEKWW | CELRKPYKCN | CRVPFGAS | EYVSFVRNQ | MRKLSYFDI |
| | | KIPPYHWSYA | ASCLNCVNYR | SSSVKQTLV | HVECCFSVS | SIDKSSAAG | SATAQIPD |
| | | HDSKYVNKSA | QSHNPKFPDI | QLIELYEMKI | FSCELRKPY | QTRKLRRLEK | GCEYVSFVR |
| | | LLTNEFCWNG | RSEGIIRTEAL | GWTELATRY | KCNSSSVK | RATARGESF | NQSIDKSSA |
| | | VISCLAELLKN | GELPSFLLSSS | LINICNGAW | QTLVQLIEL | NPALIKQRES | AGQTRKLR |
| | | VDHPLWKTLT | KIPPYHWSYA | LWENTRKAY | YEMKIGWT | IILPHYHSLEI | RLEKRATAR |
| | | KLGCYQKTRK | HDSKYVNKSA | CWNIELAPW | ELATRYLINI | DSQSKKCIFP | GESFNPALI |
| | | AMAKKLASIA | LLTNEFCWNG | PWNGNKVK | CNGAWLW | LNIQMKSEQ | KQRESIILPH |
| | | HITISMPLAPN | VISCLAELLKN | FEDIRSSYRS | ENTRKAYC | SFEGDSIFSS | YHSLEIDSQ |
| | | YLTQISLPNSD | VDHPLWKTLT | RQDFESHKD | WNIELAPW | YGLSNTDNS | SKKCIFPLNI |
| | | TSYISLSPVASL | KLGCYQKTRK | WSAITKMIK | PWNGNKV | FQPVPLI | QMKSEQSF |
| | | SMQSHFYQG | AMAKKLASIA | TAFSSSNGLA | KFEDIRSSY | (SEQ ID | EGDSIFSSY |
| | | LQDEYRHAST | HITISMPLAPN | IFEVKATLHL | RSRQDFES | NO: 329) | GLSNTDNS |
| | | TRFSRATNM | YLTQISLPNSD | PTNAMVRPS | HKDWSAIT | | FQPVPLI |
| | | GVTAMTCGG | TSYISLSPVASL | QAFTEKESG | KMIKTAFSS | | (SEQ ID |
| | | AFRMLKSNTK | SMQSHFYQG | SKSKSKSQNS | SNGLAIFEV | | NO: 330) |
| | | FSITPHHRLNS | LQDEYRHAST | RVFQSTTIDG | KATLHLPTN | | |
| | | KRSWLTSENV | TRFSRATNM | ERSPILGAFK | AMVRPSQ | | |
| | | QSLKQYQRLN | GVTAMTCGG | TGAAIATIDD | AFTEKESGS | | |
| | | KRLIPENARKA | AFRMLKSNTK | WYPGATESL | KSKSKSQNS | | |
| | | LRRKYKIEIQN | FSITPHHRLNS | RVGRFGVHR | RVFQSTTID | | |
| | | MVSVWLAM | KRSWLTSENV | EDVTCYRHP | GERSPILGA | | |
| | | QDHTLDSIILV | QSLKQYQRLN | STGKDLFSIL | FKTGAAIAT | | |
| | | QHLNHDLSCL | KRLIPENARKA | QQAEHYIEV | IDDWYPGA | | |
| | | GATKRFAYNP | LRRKYKIEIQN | LNANKTPDQ | TESLRVGRF | | |
| | | VMTKLFTELLK | MVSVWLAM | ETINDMHFL | GVHREDVT | | |
| | | RALSNSLNDS | QDHTLDSIILV | LANLIKGGM | CYRHPSTG | | |
| | | THYSNGSFLVL | QHLNHDLSCL | FQHKGD | KDLFSILQQ | | |
| | | PNIRVCGATA | GATKRFAYNP | (SEQ ID | AEHYIEVLN | | |
| | | LSSPVTVGIPS | VMTKLFTELLK | NO: 327) | ANKTPDQE | | |
| | | LTAFFGFVHA | RALSNSLNDS | | TINDMHFL | | |
| | | FERKLNRLNP | THYSNGSFLVL | | LANLIKGG | | |
| | | TFRVESFAICV | PNIRVCGATA | | MFQHKGD | | |
| | | HQLHVEKRGL | LSSPVTVGIPS | | (SEQ ID | | |
| | | TAEFVEKGNG | LTAFFGFVHA | | NO: 328) | | |
| | | TISAPATRDD | FERKLNRLNP | | | | |
| | | WQCDVVFSLI | TFRVESFAICV | | | | |
| | | LNTNFAQRID | HQLHVEKRGL | | | | |
| | | QSTLITLLPKRF | TAEFVEKGNG | | | | |
| | | ARGSAKIAIDD | TISAPATRDD | | | | |
| | | FKHINSFSTLE | WQCDVVFSLI | | | | |
| | | AAIQSLPIEAG | LNTNFAQRID | | | | |
| | | RWLSLYAQPN | QSTLITLLPKRF | | | | |
| | | NNLGDLLAA | ARGSAKIAIDD | | | | |
| | | MKEDHQLMA | FKHINSFSTLE | | | | |
| | | SCVGYHLLEEP | AAIQSLPIEAG | | | | |
| | | KDKPNSLRSY | RWLSLYAQPN | | | | |
| | | KHAFAECIIGLI | NNLGDLLAA | | | | |
| | | NSITFSSETDA | MKEDHQLMA | | | | |
| | | NTIFWSLNNH | SCVGYHLLEEP | | | | |
| | | QNYLVVQPRII | KDKPNSLRSY | | | | |
| | | NDETTDKSSL | KHAFAECIIGLI | | | | |
| | | (SEQ ID | NSITFSSETDA | | | | |
| | | NO: 325) | NTIFWSLNNH | | | | |
| | | | QNYLVVQPRII | | | | |
| | | | NDETTDKSSL | | | | |
| | | | (SEQ ID | | | | |
| | | | NO: 326) | | | | |
| 15 | Halomonas | MRQAAIIIIYQ | MPKKKRKVGS | MMNSFRHL | MPKKKRKV | MRYFFYIKYL | MPKKKRKV |
| | sp. Salt | RGNVMSLSTL | GDYKDDDDK | SYERSLNPGK | GSGMNSFR | MPSANHAFL | GSGRYFFYI |
| | Lake7 | LELDEPNRSEA | DYKDDDDKD | AVFYYRTDSS | HLSYERSLN | AGRCIACLH | KYLMPSAN |
| | | IRKAFAPYTPLI | YKDDDDKGS | EFEPLQAEVT | PGKAVFYY | GFISGPKITN | HAFLAGRCI |
| | | EVSEDVSVAIL | GRQAAIIIIYQ | RFRGPKATFS | RTDSSEFEP | SGIGVSFPS | ACLHGFISG |
| | | VLLNLSHKRKY | RGNVMSLSTL | DGYMASGT | LQAEVTRFR | WATGTVGD | PKITNSGIG |
| | | APDLLNKKRAI | LELDEPNRSEA | ARAKETSDL | GPKATFSD | SIAFVSKDIN | VSFPSWAT |
| | | ETLKDWQHM | IRKAFAPYTPLI | GFSNPIMLET | GYMASGTA | SLSYLSSARY | GTVGDSIAF |
| | | ESCAQEVQW | EVSEDVSVAIL | CYVPPLVDTL | RAKETSDLG | FKNMADEG | VSKDINSLS |
| | | VHSHNLKHPD | VLLNLSHKRKY | YCRFSLRIIAN | FSNPIMLET | FIDVSDIKMV | YLSSARYFK |
| | | TRVAHQRLLV | APDLLNKKRAI | SLEPNICDNA | CYVPPLVDT | PETLEEVRFI | NMADEGFI |
| | | KAEKPSDSIVS | ETLKDWQHM | EATKALKEFS | LYCRFSLRII | RNQHIAKSF | DVSDIKMV |
| | | SYNSVSRLGW | ESCAQEVQW | DTYRNLGGY | ANSLEPNIC | PGEIKRRLIRS | PETLEEVRFI |
| | | SHNSAAVNKA | VHSHNLKHPD | QELATRYAK | DNAEATKA | KNRAEKRGE | RNQHIAKSF |
| | | KLFGANFIFKG | TRVAHQRLLV | NILSAEWLW | LKEFSDTYR | TFMPSSAVS | PGEIKRRLIR |
| | | VVCCLAAIVLD | KAEKPSDSIVS | KNKVSRGIA | NLGGYQEL | DRFVDQCHV | SKNRAEKR |
| | | NNKQWRKEF | SYNSVSRLGW | VVVSTSNLK | ATRYAKNIL | IPIDSRSSGQ | GETFMPSS |
| | | MNLGMSGD | SHNSAAVNKA | NYCVKDAQY | SAEWLWK | RFPLYVQLEA | AVSDRFVD |
| | | QWAYLQSLF | KLFGANFIFKG | KEWGSSWE | NKVSRGIAV | LGEESKYDN | QCHVIPIDS |
| | | DNYFTKNLSP | VVCCLAAIVLD | GDELKSLEGL | VVSTSNLK | YNSYGLATQ | RSSGQRFPL |
| | | SYVDRHSVQV | NNKQWRKEF | AVEFEEALSC | NYCVKDAQ | HTHSGTVPN | YVQLEALGE |
| | | TFLYKGKDVSI | MNLGMSGD | PQKFLFADV | YKEWGSS | LKQIT (SEQ | ESKYDNYN |
| | | TPVTSHSLLAD | QWAYLQSLF | TAKIKTEFCQ | WEGDELKS | ID NO: 335) | SYGLATQH |
| | | IQIARRNKCG | DNYFTKNLSP | EIFPSQLFVE | LEGLAVEFE | | THSGTVPN |
| | | DLATIKHWHS | SYVDRHSVQV | KDDRGNGS | EALSCPQKF | | LKQIT (SEQ |
| | | SSVGDLASSL | TFLYKGKDVSI | ASRKFMKST | LFADVTAKI | | ID NO: 336) |
| | | GGNISALSYPP | TPVTSHSLLAD | MNDGRQAV | KTEFCQEIF | | |
| | | RLLACSQNKE | IQIARRNKCG | SFGAYKVGA | PSQLFVEKD | | |
| | | NENSSGIFFV | DLATIKHWHS | AIQKIDDW | DRGNGSAS | | |
| | | DFHHSSLRSKS | SSVGDLASSL | WLDEGAEYP | RKFMKSTM | | |
| | | FILACNEIVESK | GGNISALSYPP | LRVSEYGAD | NDGRQAVS | | |
| | | SLLTGKKRRD | RLLACSQNKE | RSRVLAMRE | FGAYKVGA | | |
| | | HRRSAIKLLRQ | NENSSGIFFV | PVTKKDFYSL | AIQKIDDW | | |
| | | SLSEWLSPVSY | DFHHSSLRSKS | LNEIINITEE | WLDEGAEY | | |
| | | WRSVGGEVLS | FILACNEIVESK | MIKTRQASP | PLRVSEYGA | | |
| | | ERQNNSACLLI | SLLTGKKRRD | NAHYVMSVL | DRSRVLAM | | |
| | | SAPNEDLLEIL | HRRSAIKLLRQ | VKGGMFQK | REPVTKKDF | | |
| | | PEVNKELHSIL | SLSEWLSPVSY | GIKKGEK | YSLLNEIINI | | |
| | | VRYPQTQSFA | WRSVGGEVLS | (SEQ ID | TEEMIKTR | | |
| | | YHPELLIPFKA | ERQNNSACLLI | NO: 333) | QASPNAHY | | |
| | | QLKSLLIGMKI | SAPNEDLLEIL | | VMSVLVKG | | |
| | | KDDEPMAEE | PEVNKELHSIL | | GMFQKGIK | | |
| | | PYHYLHLTNL | VRYPQTQSFA | | KGEK (SEQ | | |
| | | HVFDAQALSC | YHPELLIPFKA | | ID NO: 334) | | |
| | | PYLVGLPSLLA | QLKSLLIGMKI | | | | |
| | | VWGTVYNYQ | KDDEPMAEE | | | | |
| | | LRLRNILKRNI | PYHYLHLTNL | | | | |
| | | VFEGVAWFLR | HVFDAQALSC | | | | |
| | | QYESSSGAKIP | PYLVGLPSLLA | | | | |
| | | APYLPPMKPG | VWGTVYNYQ | | | | |
| | | ETPKRPGLID | LRLRNILKRNI | | | | |
| | | MRFCDLRMD | VFEGVAWFLR | | | | |
| | | LVICYRLEDGD | QYESSSGAKIP | | | | |
| | | DTPLGNDELT | APYLPPMKPG | | | | |
| | | MLQSAFPGRF | ETPKRPGLID | | | | |
| | | AGGTMQPPP | MRFCDLRMD | | | | |
| | | LYEELQWCQL | LVICYRLEDGD | | | | |
| | | HGDANSLLAA | DTPLGNDELT | | | | |
| | | ISLLPDEGRW | MLQSAFPGRF | | | | |
| | | VVDSEKQVQS | AGGTMQPPP | | | | |
| | | IDSLVAWLTK | LYEELQWCQL | | | | |
| | | HPNHLPAMS | HGDANSLLAA | | | | |
| | | GYQLLEEPCY | ISLLPDEGRW | | | | |
| | | RSGSHRELHA | VVDSEKQVQS | | | | |
| | | YAEPLVGLTET | IDSLVAWLTK | | | | |
| | | LSPASVRLNG | HPNHLPAMS | | | | |
| | | KADFLKNAF | GYQLLEEPCY | | | | |
| | | WRLKSQNLT | RSGSHRELHA | | | | |
| | | MLMKKA | YAEPLVGLTET | | | | |
| | | (SEQ ID | LSPASVRLNG | | | | |
| | | NO: 331) | KADFLKNAF | | | | |
| | | | WRLKSQNLT | | | | |
| | | | MLMKKA | | | | |
| | | | (SEQ ID | | | | |
| | | | NO: 332) | | | | |
| 16 | V.EJY3- | MKLSDVLRIE | MPKKKRKVGS | MELCRQLNY | MPKKKRKV | MERRYYFSIR | MPKKKRKV |
| | NC_016614 | DEVLKQTTFK | GDYKDDDDK | LRSISPGKAY | GSGELCRQ | YVPSYADFG | GSGERRYYF |
| | | KVFMPYSEDI | DYKDDDDKD | FYYLASNGD | LNYLRSISP | LLAGRCIYQ | SIRYVPSYA |
| | | EIDGCEKEALII | YKDDDDKGS | RCPLAIDKTH | GKAYFYYLA | MHLFSVNNP | DFGLLAGR |
| | | LLNLSYYPKGT | GKLSDVLRIED | IRAPKGGYA | SNGDRCPL | EVKNKVGVC | CIYQMHLFS |
| | | KHINWLDDER | EVLKQTTFKK | EAYQGSSFV | AIDKTHIRA | FPRWNSKDV | VNNPEVKN |
| | | ALDYLTEQDN | VFMPYSEDIEI | KKNVAPQDL | PKGGYAEA | GDMIAFVM | KVGVCFPR |
| | | LTASLAEVQW | DGCEKEALIILL | SYSNPQFIEE | YQGSSFVK | EDKEALLGLA | WNSKDVG |
| | | FHTHNLKYPD | NLSYYPKGTK | CYVPPLTNEII | KNVAPQDL | FQPYFSRMT | DMIAFVME |
| | | CRVSKQKIIGE | HINWLDDER | CEFSLRIRAN | SYSNPQFIE | KEGVFELSKV | DKEALLGLA |
| | | PLPADDVFISS | ALDYLTEQDN | SLHPDVCSD | ECYVPPLTN | DEVPKSSSEV | FQPYFSRM |
| | | ATLKPILGWA | LTASLAEVQW | EKVREQLMS | EIICEFSLRIR | RFVRNQAIG | TKEGVFELS |
| | | HNSAAYRYTI | FHTHNLKYPD | LAKVYKELN | ANSLHPDV | KSFIASKKRRI | KVDEVPKSS |
| | | WLLNSFIWQS | CRVSKQKIIGE | GYQELAYRY | CSDEKVRE | KRSMTRAEL | SEVRFVRN |
| | | QPTNILTLIEQ | PLPADDVFISS | AKNILLGSW | QLMSLAKV | LDFEHTPVA | QAIGKSFIA |
| | | QNPIWLDLLR | ATLKPILGWA | LWRNKDCR | YKELNGYQ | VEERVVEHY | SKKRRIKRS |
| | | AFGLREKSLEL | HNSAAYRYTI | GVTIQVMTS | ELAYRYAKN | HRIPISSGSS | MTRAELLD |
| | | LRTEIELQLSS | WLLNSFIWQS | DGESIEVYDA | ILLGSWLW | GQDYILHIQK | FEHTPVAV |
| | | QSFPRYVDSY | QPTNILTLIEQ | TKLSWYGH | RNKDCRGV | ERVESRGQQ | EERVVEHY |
| | | SKQLRFPWN | QNPIWLDLLR | WDEQSTQSL | TIQVMTSD | DFSSYGLATK | HRIPISSGSS |
| | | GDYLSVTPVV | AFGLREKSLEL | EQLTSYLSRA | GESIEVYDA | QEKRGTVPA | GQDYILHIQ |
| | | SHAMQRELE | LRTEIELQLSS | LSDRSQCFY | TKLSWYGH | LYI (SEQ ID | KERVESRG |
| | | HRYRNAESHL | QSFPRYVDSY | MDVKAVMS | WDEQSTQS | NO: 341) | QQDFSSYG |
| | | KFVTLSFPNSA | SKQLRFPWN | VGRGDEVYP | LEQLTSYLS | | LATKQEKR |
| | | SIGNLCGSVG | GDYLSVTPVV | SQEFIDVKQE | RALSDRSQ | | GTVPALYI |
| | | GNMQVLNYP | SHAMQRELE | GIPTRQLAKV | CFYMDVKA | | (SEQ ID |
| | | LDVPSSTNRST | HRYRNAESHL | PLNYEQETA | VMSVGRG | | NO: 342) |
| | | LRKTLADSRLA | KFVTLSFPNSA | AFHAQKIGA | DEVYPSQEF | | |
| | | SGRYFDDFQL | SIGNLCGSVG | ALQSIDDW | IDVKQEGIP | | |
| | | TNERICKVLSR | GNMQVLNYP | WHENADKP | TRQLAKVPL | | |
| | | LTGTETSTTHK | LDVPSSTNRST | LRVNEYGAD | NYEQETAA | | |
| | | RRIKSRKDQSR | LRKTLADSRLA | REYVIARRHS | FHAQKIGA | | |
| | | ILRKQVALW | SGRYFDDFQL | LLGNDFYQLI | ALQSIDDW | | |
| | | MLPLIELRDRF | TNERICKVLSR | RRTEKWIEE | WHENADK | | |
| | | DSDEREGVIEE | LTGTETSTTHK | MDKSKSIPN | PLRVNEYG | | |
| | | HESLVQDFLTL | RRIKSRKDQSR | DVHFILSVLIK | ADREYVIAR | | |
| | | SESDLPVLVSQ | ILRKQVALW | GGLFNCSKT | RHSLLGND | | |
| | | FNQRLHYVFQ | MLPLIELRDRF | KSKSKSKSK | FYQLIRRTE | | |
| | | ENKFTRKFAY | DSDEREGVIEE | (SEQ ID | KWIEEMDK | | |
| | | HPKLLQVVKS | HESLVQDFLTL | NO: 339) | SKSIPNDVH | | |
| | | QIVWVLNKLS | SESDLPVLVSQ | | FILSVLIKGG | | |
| | | KPQEDEVSGQ | FNQRLHYVFQ | | LFNCSKTKS | | |
| | | GEQYIYLSSLR | ENKFTRKFAY | | KSKSKSK | | |
| | | VQDSLAMSC | HPKLLQVVKS | | (SEQ ID | | |
| | | PYLCGVPSLTA | QIVWVLNKLS | | NO: 340) | | |
| | | IWGFVHHYQ | KPQEDEVSGQ | | | | |
| | | REFNRSINSD | GEQYIYLSSLR | | | | |
| | | VFYEFAGFSIY | VQDSLAMSC | | | | |
| | | VRSQSITVGA | PYLCGVPSLTA | | | | |
| | | KLTEPNSVEK | IWGFVHHYQ | | | | |
| | | VRTLSNAKRP | REFNRSINSD | | | | |
| | | TIRTDRFADLE | VFYEFAGFSIY | | | | |
| | | IDLVICVKSNG | VRSQSITVGA | | | | |
| | | RLSDYRAALKS | KLTEPNSVEK | | | | |
| | | VLPLSLAGGSL | VRTLSNAKRP | | | | |
| | | FQPLISSKIDW | TIRTDRFADLE | | | | |
| | | LRTFDSQSSLF | IDLVICVKSNG | | | | |
| | | HALKGLPAYG | RLSDYRAALKS | | | | |
| | | RWLYPCELQP | VLPLSLAGGSL | | | | |
| | | DSFDELESTLD | FQPLISSKIDW | | | | |
| | | QNSGCLPVSN | LRTFDSQSSLF | | | | |
| | | GYHFLEIPIHR | HALKGLPAYG | | | | |
| | | NNALTALHTY | RWLYPCELQP | | | | |
| | | AENTLTVAKQ | DSFDELESTLD | | | | |
| | | VIPIEMRFAGS | QNSGCLPVSN | | | | |
| | | KQFFQEAFWS | GYHFLEIPIHR | | | | |
| | | LECSSTTILVKK | NNALTALHTY | | | | |
| | | YKE (SEQ ID | AENTLTVAKQ | | | | |
| | | NO: 337) | VIPIEMRFAGS | | | | |
| | | | KQFFQEAFWS | | | | |
| | | | LECSSTTILVKK | | | | |
| | | | YKE (SEQ ID | | | | |
| | | | NO: 338) | | | | |
| 17 | Photo_ | MKKLCDVLQI | MPKKKRKVGS | MELCNQLNY | MPKKKRKV | MTTRYYFTIQ | MPKKKRKV |
| | aquaeCGMCC | EDNTEKQATL | GDYKDDDDK | VRSLSAGKA | GSGELCNQ | YIPTHADFGL | GSGTTRYYF |
| | | KKVFMPYSAC | DYKDDDDKD | YFYHLSKGGE | LNYVRSLSA | LAGRCIYQM | TIQYIPTHA |
| | | IDIDGCEKEAL | YKDDDDKGS | MCPLEIDRT | GKAYFYHLS | HKFMVNNP | DFGLLAGR |
| | | TVLLNLSTHRK | GKKLCDVLQIE | RLRAPKGGY | KGGEMCPL | LAMNQIGVS | CIYQMHKF |
| | | GSPCGDWLDI | DNTEKQATLK | AEAYKGSKF | EIDRTRLRA | FPMWEDGS | MVNNPLA |
| | | ERAKSYLKDQ | KVFMPYSACI | VQKNVAPQ | PKGGYAEA | VGNIIAFISED | MNQIGVSF |
| | | ADIDASLAEIK | DIDGCEKEALT | DLAYANPQF | YKGSKFVQ | KELMVGLLF | PMWEDGS |
| | | WFHTHNLKFP | VLLNLSTHRK | IEECYVKPGV | KNVAPQDL | QPYFSLMVK | VGNIIAFISE |
| | | DCRVKEQRLI | GSPCGDWLDI | DDIYCAFSLRI | AYANPQFIE | EGLFEISSVC | DKELMVGL |
| | | AKPLSTSESFIS | ERAKSYLKDQ | KANSLGPDV | ECYVKPGV | EVPTDSPEV | LFQPYFSLM |
| | | SVSLDQGLG | ADIDASLAEIK | CCDDEVRSK | DDIYCAFSL | RFVRNQTIG | VKEGLFEISS |
| | | WAHNSAVYR | WFHTHNLKFP | LSSLAKSYKE | RIKANSLGP | KSFIGSKKRRI | VCEVPTDSP |
| | | HTLWLLNSFN | DCRVKEQRLI | LSGYSELAHR | DVCCDDEV | KRSMARAEL | EVRFVRNQ |
| | | WQSESVNILS | AKPLSTSESFIS | YAKNILLGT | RSKLSSLAK | SGAEYSLPVA | TIGKSFIGSK |
| | | LVQEENPVW | SVSLDQGLG | WLWRNREC | SYKELSGYS | VEERVVDHF | KRRIKRSM |
| | | LELLQEFGLNI | WAHNSAVYR | RRLSIEVTTS | ELAHRYAK | HRVPISSGSS | ARAELSGAE |
| | | KQQDLLLKTIE | HTLWLLNSFN | DSETLIVENA | NILLGTWL | GHDYILHIQK | YSLPVAVEE |
| | | LQIPASTFPDS | WQSESVNILS | TKLTWYDH | WRNRECRR | EVASERSVA | RVVDHFHR |
| | | VSPYSKQLRFP | LVQEENPVW | WDKDAAEC | SIEVTTSDS | NFNSYGLAT | VPISSGSSG |
| | | WNNDYLSVT | LELLQEFGLNI | LDKLTAYLTR | ETLIVENAT | NQEKRGTVP | HDYILHIQK |
| | | PVVSHAIQREI | KQQDLLLKTIE | ALSDPTEYFY | KLTWYDH | DLCI | EVASERSVA |
| | | EVKARDKASK | LQIPASTFPDS | MDVKAKIAV | WDKDAAE | (SEQ ID | NFNSYGLA |
| | | LSFVTSALPNS | VSPYSKQLRFP | GWGDEVYP | CLDKLTAYL | NO: 347) | TNQEKRGT |
| | | ASIGNLCGSLG | WNNDYLSVT | SQEFLDNRE | TRALSDPTE | | VPDLCI |
| | | GYMKALNYPL | PVVSHAIQREI | DGVPTKQLA | YFYMDVKA | | (SEQ ID |
| | | DVKSVAEQTL | EVKARDKASK | TVELENGRE | KIAVGWGD | | NO: 348) |
| | | AASRNKSGKY | LSFVTSALPNS | TVAFHGQKV | EVYPSQEFL | | |
| | | FDDFQVTNYK | ASIGNLCGSLG | GAALQSIDD | DNREDGVP | | |
| | | ICQVLNRLIGA | GYMKALNYPL | WWHEKADK | TKQLATVEL | | |
| | | EPLKNQKQRE | DVKSVAEQTL | PLRVNEYGA | ENGRETVA | | |
| | | KARKVQSKILR | AASRNKSGKY | DREYVIARR | FHGQKVGA | | |
| | | KQIALWMLPL | FDDFQVTNYK | HVSLKNDFY | ALQSIDDW | | |
| | | IELRDIEDAEP | ICQVLNRLIGA | QLLRNTENW | WHEKADKP | | |
| | | HNQQLEHDD | EPLKNQKQRE | IESMNTSNII | LRVNEYGA | | |
| | | PLVKSFLSLPE | KARKVQSKILR | PNDVHFIMS | DREYVIARR | | |
| | | SEFPSLVHELN | KQIALWMLPL | VLVKGGLFN | HVSLKNDF | | |
| | | QRLHFVFQEN | IELRDIEDAEP | CSKSKSK | YQLLRNTE | | |
| | | KFTAKFAYHP | HNQQLEHDD | (SEQ ID | NWIESMNT | | |
| | | KLIQVVKAQIV | PLVKSFLSLPE | NO: 345) | SNIIPNDVH | | |
| | | WVLEQLSKPS | SEFPSLVHELN | | FIMSVLVKG | | |
| | | DHEDAAREQ | QRLHFVFQEN | | GLFNCSKSK | | |
| | | QYIYLSSLRVQ | KFTAKFAYHP | | SK (SEQ ID | | |
| | | DAVAMSSPYL | KLIQVVKAQIV | | NO: 346) | | |
| | | CGAPSLTAIW | WVLEQLSKPS | | | | |
| | | GFMHHYQRE | DHEDAAREQ | | | | |
| | | FNKLVNSDSP | QYIYLSSLRVQ | | | | |
| | | FEFSRFAFYVR | DAVAMSSPYL | | | | |
| | | TENIQSTAKLT | CGAPSLTAIW | | | | |
| | | EPNSLAKSRTL | GFMHHYQRE | | | | |
| | | SNAKRPTIRSE | FNKLVNSDSP | | | | |
| | | RLADLEIDLVI | FEFSRFAFYVR | | | | |
| | | RVDSDSRISDF | TENIQSTAKLT | | | | |
| | | LSELRAALPAA | EPNSLAKSRTL | | | | |
| | | FAGGALYQPLI | SNAKRPTIRSE | | | | |
| | | LSQIDWLRTF | RLADLEIDLVI | | | | |
| | | SSKSELFHVLK | RVDSDSRISDF | | | | |
| | | GIPAYGSWLY | LSELRAALPAA | | | | |
| | | PSEKQPTNFN | FAGGALYQPLI | | | | |
| | | ELEHLITEDAD | LSQIDWLRTF | | | | |
| | | NLPVSIGYHLL | SSKSELFHVLK | | | | |
| | | EHPTERENSIT | GIPAYGSWLY | | | | |
| | | DCHAYAENAL | PSEKQPTNFN | | | | |
| | | GIAKRLNPIEV | ELEHLITEDAD | | | | |
| | | RFSGRDHFFD | NLPVSIGYHLL | | | | |
| | | NAFWALESTS | EHPTERENSIT | | | | |
| | | ATILIKNDRN | DCHAYAENAL | | | | |
| | | (SEQ ID | GIAKRLNPIEV | | | | |
| | | NO: 343) | RFSGRDHFFD | | | | |
| | | | NAFWALESTS | | | | |
| | | | ATILIKNDRN | | | | |
| | | | (SEQ ID | | | | |
| | | | NO: 344) | | | | |
| 18 | Enterovibrio | MKTLRDVLED | MPKKKRKVGS | MNGLTGELA | MPKKKRKV | MKRYYFVITY | MPKKKRKV |
| | coralii | EEPDIALRKAF | GDYKDDDDK | SALSGEEPF | GSGNGLTG | LPEQASQEIL | GSGKRYYF |
| | strain CAIM | AAYSELVDVT | DYKDDDDKD | WLADIKANV | ELASALSGE | AGRCISTLHD | VITYLPEQA |
| | 912 | GEETQTLIVLL | YKDDDDKGS | SASFMQEIFP | EPFWLADIK | FLVFHHIGGI | SQEILAGRC |
| | | NLTLKRDEVES | GKTLRDVLED | SQLFSDAKD | ANVSASFM | GVGFPKWTE | ISTLHDFLVF |
| | | LTSRKSARAVL | EEPDIALRKAF | GSNLGREYA | QEIFPSQLF | QSLGNQIMF | HHIGGIGV |
| | | KDEAHIDSCLE | AAYSELVDVT | KVRSGDGQI | SDAKDGSN | CSTNQQRLS | GFPKWTEQ |
| | | EVRWLHSHN | GEETQTLIVLL | WPSLNAEKI | LGREYAKV | QLHQSKYFT | SLGNQIMF |
| | | LKYPDTRVQA | NLTLKRDEVES | GAAIQLIDD | RSGDGQIW | MMFDQGLF | CSTNQQRL |
| | | QRILCGDLPLI | LTSRKSARAVL | WWADEADK | PSLNAEKIG | AVTDVEPVP | SQLHQSKY |
| | | AGVLGSANCE | KDEAHIDSCLE | RLRVHEYGG | AAIQLIDD | ADTAEVRFY | FTMMFDQ |
| | | RRLGWSHNS | EVRWLHSHN | DKKYHIAHRI | WWADEAD | RNQGIAKLFT | GLFAVTDV |
| | | SQVNKAKLFC | LKYPDTRVQA | PSSGIDAYSL | KRLRVHEY | GEKRRRLER | EPVPADTA |
| | | SGFIWEGSST | QRILCGDLPLI | LKSVDDKAA | GGDKKYHI | AKRRAAERG | EVRFYRNQ |
| | | CLAESVIKNSD | AGVLGSANCE | LLDSLKCSDEI | AHRIPSSGI | EMFDPERIG | GIAKLFTGE |
| | | AWRRAFREF | RRLGWSHNS | PSDIHYLMAI | DAYSLLKSV | SNQPIGMFH | KRRRLERAK |
| | | GLTKTKFEEW | SQVNKAKLFC | LVKGGLFQK | DDKAALLD | RILMDSQST | RRAAERGE |
| | | RLQLKQVMN | SGFIWEGSST | SRSA (SEQ | SLKCSDEIPS | QQRFVLHVQ | MFDPERIG |
| | | TDHFPSEVSD | CLAESVIKNSD | ID NO: 351) | DIHYLMAIL | KEDVAEASG | SNQPIGMF |
| | | YSKQVRFPWL | AWRRAFREF | | VKGGLFQK | TDFNGYGLA | HRILMDSQ |
| | | SDYFAITPVVS | GLTKTKFEEW | | SRSA (SEQ | TNRAYRGTV | STQQRFVL |
| | | SAVLAKIQQL | RLQLKQVMN | | ID NO: 352) | PDIRIPV | HVQKEDVA |
| | | RTQRLGHFRQ | TDHFPSEVSD | | | (SEQ ID | EASGTDFN |
| | | IDHCHPASVG | YSKQVRFPWL | | | NO: 353) | GYGLATNR |
| | | DFAASRGGG | SDYFAITPVVS | | | | AYRGTVPDI |
| | | VTVLNYPLNIV | SAVLAKIQQL | | | | RIPV (SEQ |
| | | WRNHVSLNQ | RTQRLGHFRQ | | | | ID NO: 354) |
| | | SRIRRVESDKS | IDHCHPASVG | | | | |
| | | AFNSWALLNE | DFAASRGGG | | | | |
| | | RFIGVLNSLIH | VTVLNYPLNIV | | | | |
| | | LDEEPVLRRR | WRNHVSLNQ | | | | |
| | | RRRRVSLVRQ | SRIRRVESDKS | | | | |
| | | LRRGIAEWLL | AFNSWALLNE | | | | |
| | | PIMEWRDSLR | RFIGVLNSLIH | | | | |
| | | DGADTLAAIR | LDEEPVLRRR | | | | |
| | | ETERALLTEPL | RRRRVSLVRQ | | | | |
| | | SDNTKLLKLV | LRRGIAEWLL | | | | |
| | | NQRFHTTLQD | PIMEWRDSLR | | | | |
| | | AGYRNTEYAY | DGADTLAAIR | | | | |
| | | HPKLLEPVRN | ETERALLTEPL | | | | |
| | | QLRWILDTLG | SDNTKLLKLV | | | | |
| | | NDQFGQRNT | NQRFHTTLQD | | | | |
| | | QFEVIHLENLR | AGYRNTEYAY | | | | |
| | | VFDALSLANP | HPKLLEPVRN | | | | |
| | | YLVGIPSLTAL | QLRWILDTLG | | | | |
| | | WGFIHAFDRK | NDQFGQRNT | | | | |
| | | LKTLLGCEFTF | QFEVIHLENLR | | | | |
| | | ESVAWHVRES | VFDALSLANP | | | | |
| | | SSVSGLKLPSP | YLVGIPSLTAL | | | | |
| | | ALERKRSDHL | WGFIHAFDRK | | | | |
| | | KRPGMIESKH | LKTLLGCEFTF | | | | |
| | | CDLVMDLAIR | ESVAWHVRES | | | | |
| | | VHSTEQFLQT | SSVSGLKLPSP | | | | |
| | | RDELVDLIKAA | ALERKRSDHL | | | | |
| | | LPSRFAGGVI | KRPGMIESKH | | | | |
| | | HPPSLYESRD | CDLVMDLAIR | | | | |
| | | WCSLRTTQSL | VHSTEQFLQT | | | | |
| | | HEHVSRLPAT | RDELVDLIKAA | | | | |
| | | GRWIVPATTT | LPSRFAGGVI | | | | |
| | | PKSFENLCELV | HPPSLYESRD | | | | |
| | | ELNSDLKPAM | WCSLRTTQSL | | | | |
| | | LGYQLLEEPIE | HEHVSRLPAT | | | | |
| | | RPNSVASLHA | GRWIVPATTT | | | | |
| | | YAEPLIGLCDC | PKSFENLCELV | | | | |
| | | KSSIDIRLKGE | ELNSDLKPAM | | | | |
| | | KYFNANFFWK | LGYQLLEEPIE | | | | |
| | | MDTATSSILM | RPNSVASLHA | | | | |
| | | RRA (SEQ ID | YAEPLIGLCDC | | | | |
| | | NO: 349) | KSSIDIRLKGE | | | | |
| | | | KYFNANFFWK | | | | |
| | | | MDTATSSILM | | | | |
| | | | RRA (SEQ ID | | | | |
| | | | NO: 350) | | | | |
| 19 | Vibrio | MESLKELLQS | MPKKKRKVGS | MELPTNLAY | MPKKKRKV | MKWYYKTV | MPKKKRKV |
| | chagasii | RPDDLSVDLK | GDYKDDDDK | ERSIDPSDVC | GSGELPTNL | TFLPARCNN | GSGKWYYK |
| | strain | RAFRPLTPHIN | DYKDDDDKD | FLVVWPDGR | AYERSIDPS | ESLAAKCLRIL | TVTFLPARC |
| | ECSMB14107 | IDGKELDALTV | YKDDDDKGS | KTPLTYTSRT | DVCFLVVW | HGFNYEYET | NNESLAAK |
| | | LVNLTDKTAD | GESLKELLQSR | VLGQMETA | PDGRKTPLT | RNIGVSFPL | CLRILHGFN |
| | | QKDLLDKVKC | PDDLSVDLKR | ALAYDPSGKI | YTSRTVLGQ | WSDDTIGNK | YEYETRNIG |
| | | KQKLRDEKW | AFRPLTPHINI | KESATAEILA | METAALAY | ISFVSTNKIEL | VSFPLWSD |
| | | WARCLKTVEY | DGKELDALTV | QGNLHQVD | DPSGKIKES | DLLLKQHYFT | DTIGNKISF |
| | | RQSHNLKFPD | LVNLTDKTAD | FCHAPFGAS | ATAEILAQG | QMKDLHYF | VSTNKIELD |
| | | IRSEGVIRATP | QKDLLDKVKC | HIECYFSVSF | NLHQVDFC | DISNTKVVP | LLLKQHYFT |
| | | LGQLPDFLLSS | KQKLRDEKW | SSELRKPYKC | HAPFGASHI | DGCEYVSFK | QMKDLHYF |
| | | SKLEPHNWAY | WARCLKTVEY | NSSTVKHTL | ECYFSVSFS | RCQSIDKATP | DISNTKVVP |
| | | SHDSSDVNKS | RQSHNLKFPD | MQLIKAYEE | SELRKPYKC | AGQARKAKR | DGCEYVSFK |
| | | ALLTNEFRWN | IRSEGVIRATP | NIGWNELVS | NSSTVKHTL | LKKRAEERGE | RCQSIDKAT |
| | | GVISCLGDLLR | LGQLPDFLLSS | RYLVNICNGS | MQLIKAYEE | EFDLSSFKQH | PAGQARKA |
| | | DVEHPLWQK | SKLEPHNWAY | WLWKNTKK | NIGWNELV | EVVALHHYH | KRLKKRAEE |
| | | FNTLGCYQKT | SHDSSDVNKS | AYCWDIELT | SRYLVNICN | SLEEDSKSRG | RGEEFDLSS |
| | | RKAIAKKLAQI | ALLTNEFRWN | PWPWAGG | GSWLWKN | GSFRLNIRIFK | FKQHEVVA |
| | | SQTTINVSLAP | GVISCLGDLLR | AVKFQDIRA | TKKAYCWD | EARLDGDAL | LHHYHSLEE |
| | | NYLTQLSLPD | DVEHPLWQK | NYLERSDFE | IELTPWPW | FSSYGLANTE | DSKSRGGS |
| | | NDSSYISLSPV | FNTLGCYQKT | NHKDWEAIA | AGGAVKFQ | NTSQPVPII | FRLNIRIFKE |
| | | ASQSMQSHC | RKAIAKKLAQI | QMTRNAFS | DIRANYLER | (SEQ ID | ARLDGDAL |
| | | YQALENEYRY | SQTTINVSLAP | HSNGLAIFEV | SDFENHKD | NO: 359) | FSSYGLANT |
| | | TALTRYSRSTN | NYLTQLSLPD | KATLRLPTNK | WEAIAQM | | ENTSQPVPI |
| | | MGVLPMTCG | NDSSYISLSPV | QIFPSQAFTE | TRNAFSHS | | I (SEQ ID |
| | | GALKMLKAVP | ASQSMQSHC | NESNNTNKS | NGLAIFEVK | | NO: 360) |
| | | NFSLAPHYQI | YQALENEYRY | KKKSKGRIFQ | ATLRLPTNK | | |
| | | NIGKFWLTSS | TALTRYSRSTN | STTVDGERS | QIFPSQAFT | | |
| | | HIQSLKQYQR | MGVLPMTCG | PILGIYKTGA | ENESNNTN | | |
| | | HTRYLMPENK | GALKMLKAVP | AIATIDDWY | KSKKKSKGR | | |
| | | RIAYRRTVENE | NFSLAPHYQI | PDATEALRV | IFQSTTVDG | | |
| | | IHEMVKAWL | NIGKFWLTSS | GRFGVHKED | ERSPILGIYK | | |
| | | ATQDNTMDV | HIQSLKQYQR | VTCYRHPST | TGAAIATID | | |
| | | NTLVQHLND | HTRYLMPENK | QKDFFSILKQ | DWYPDATE | | |
| | | DLSRFKSAKCF | RIAYRRTVENE | TESYIEALTSS | ALRVGRFG | | |
| | | AYEPNITKLLL | IHEMVKAWL | DKPNQETIN | VHKEDVTC | | |
| | | GLIKRELTEPT | ATQDNTMDV | DLHFLVANII | YRHPSTQK | | |
| | | TVSTNICRSEE | NTLVQHLND | KGGMFQHK | DFFSILKQT | | |
| | | KNSFFAIPNIR | DLSRFKSAKCF | GD (SEQ ID | ESYIEALTSS | | |
| | | VCGASALSSPI | AYEPNITKLLL | NO: 357) | DKPNQETI | | |
| | | TVGLPSLTAFL | GLIKRELTEPT | | NDLHFLVA | | |
| | | GFTHAFERNL | TVSTNICRSEE | | NIIKGGMF | | |
| | | NESFPTLAIDS | KNSFFAIPNIR | | QHKGD | | |
| | | FAICIHQLHIE | VCGASALSSPI | | (SEQ ID | | |
| | | KRGLTKEYVQ | TVGLPSLTAFL | | NO: 358) | | |
| | | KANHTISPPAT | GFTHAFERNL | | | | |
| | | HDDWQCDLV | NESFPTLAIDS | | | | |
| | | FSLVIKFNRSL | FAICIHQLHIE | | | | |
| | | NVDENTIVRA | KRGLTKEYVQ | | | | |
| | | LPKRFARGSA | KANHTISPPAT | | | | |
| | | KIAIADFKYIRS | HDDWQCDLV | | | | |
| | | FSTLEKTIQSF | FSLVIKFNRSL | | | | |
| | | PQKAGKWLS | NVDENTIVRA | | | | |
| | | MHTEPIKNM | LPKRFARGSA | | | | |
| | | SDILSEVKENR | KIAIADFKYIRS | | | | |
| | | KLTPSCVGYH | FSTLEKTIQSF | | | | |
| | | FLEEPTDKPNS | PQKAGKWLS | | | | |
| | | LRGYKHAFSE | MHTEPIKNM | | | | |
| | | CIIGLIEPITFD | SDILSEVKENR | | | | |
| | | QNTDINTILW | KLTPSCVGYH | | | | |
| | | HHKCYQNYLS | FLEEPTDKPNS | | | | |
| | | VQPRSTYHGT | LRGYKHAFSE | | | | |
| | | TD (SEQ ID | CIIGLIEPITFD | | | | |
| | | NO: 355) | QNTDINTILW | | | | |
| | | | HHKCYQNYLS | | | | |
| | | | VQPRSTYHGT | | | | |
| | | | TD (SEQ ID | | | | |
| | | | NO: 356) | | | | |
| 20 | Vibrio | MRTLAEILKSE | MPKKKRKVGS | MKLPNSLSY | MPKKKRKV | MDWYYKTIT | MPKKKRKV |
| | rotiferianus | TDDLNRDLRR | GDYKDDDDK | MRSIDPSDT | GSGKLPNSL | FLPEYRNNE | GSGDWYYK |
| | CAIM 577 | AFRPLSPPVDI | DYKDDDDKD | VFFVNWPN | SYMRSIDPS | AIAAKCLKEL | TITFLPEYR |
| | APHW01000105 | SDFPSEALTILI | YKDDDDKGS | GKRTPLPYSS | DTVFFVNW | HSFNYEYKTR | NNEAIAAK |
| | | NLTDTVKEQK | GRTLAEILKSE | RTALGRKEG | PNGKRTPL | SIGISFPLWN | CLKELHSFN |
| | | ELLDRSKCKEK | TDDLNRDLRR | TSSAYKNDD | PYSSRTALG | QETVGQKIT | YEYKTRSIGI |
| | | LRDEKWWLS | AFRPLSPPVDI | EINEDVTEYS | RKEGTSSAY | FVSTNKMEL | SFPLWNQE |
| | | CLKTVKYRQS | SDFPSEALTILI | LAHGNPHEI | KNDDEINE | DFLLSRRYFT | TVGQKITFV |
| | | HNPKFPDIRA | NLTDTVKEQK | DYCCVPYGA | DVTEYSLAH | QMTKLGYFS | STNKMELD |
| | | SGIIRAIPMGD | ELLDRSKCKEK | ESIECEFSVSF | GNPHEIDY | ISTAQIVPDD | FLLSRRYFT |
| | | IPPFMLSSSKL | LRDEKWWLS | ASSLRKPFKC | CCVPYGAE | CSYALFRRKQ | QMTKLGYF |
| | | ARCNWAYAN | CLKTVKYRQS | SDPQVKRTLI | SIECEFSVSF | SIDKATPAG | SISTAQIVP |
| | | DSSQVNKSSF | HNPKFPDIRA | QLIELYEQKV | ASSLRKPFK | QARELKRLER | DDCSYALFR |
| | | LTSEFIWHNR | SGIIRAIPMGD | GWEELATRF | CSDPQVKR | RALERGEIFE | RKQSIDKAT |
| | | VHFLGELLTDI | IPPFMLSSSKL | LENICNGRW | TLIQLIELYE | PANYSQNTT | PAGQAREL |
| | | EHPLWNILKN | ARCNWAYAN | LWRNNERTY | QKVGWEEL | HAFHNYHSL | KRLERRALE |
| | | LGCYVKTSKEI | DSSQVNKSSF | STSISIKPWP | ATRFLENIC | EENSSGGNG | RGEIFEPAN |
| | | SKKLALIPPHEI | LTSEFIWHNR | WKDEEVIISF | NGRWLWR | FRLNIQMEQ | YSQNTTHA |
| | | STPLARNYLT | VHFLGELLTDI | NDIRRNYTDI | NNERTYSTS | LEDTLSTGKF | FHNYHSLEE |
| | | QISLPDNEDSY | EHPLWNILKN | NKFRDHED | ISIKPWPW | SSYGLGNTD | NSSGGNGF |
| | | ISLSPVTSQSI | LGCYVKTSKEI | WEALIKLITD | KDEEVIISFN | NSLQVVPLI | RLNIQMEQ |
| | | QNNCYETLKE | SKKLALIPPHEI | AFSKPNGLCI | DIRRNYTDI | (SEQ ID | LEDTLSTGK |
| | | HYRFSSLTRFS | STPLARNYLT | FEVNATFRL | NKFRDHED | NO: 365) | FSSYGLGNT |
| | | RATNMGTLA | QISLPDNEDSY | GKNAPIYPS | WEALIKLIT | | DNSLQVVP |
| | | MSCGGNFRM | ISLSPVTSQSI | QVFKESIQGE | DAFSKPNG | | LI (SEQ ID |
| | | IHSLPPIEKYK | QNNCYETLKE | KNRIYQKTEV | LCIFEVNAT | | NO: 366) |
| | | HHHLTDAEQ | HYRFSSLTRFS | CGEKSPILGC | FRLGKNAPI | | |
| | | WLTKKSVKAL | RATNMGTLA | YKTGAAIATI | YPSQVFKES | | |
| | | REYTESTHWII | MSCGGNFRM | DDWYHPDA | IQGEKNRIY | | |
| | | SPNKLAKKRK | IHSLPPIEKYK | EEPLRISHYG | QKTEVCGE | | |
| | | SIIENIRLMLT | HHHLTDAEQ | AHKEDVYCY | KSPILGCYK | | |
| | | QWLNTISERE | WLTKKSVKAL | RHPNTGKDL | TGAAIATID | | |
| | | YSNKKELTERF | REYTESTHWII | FTLLQRADEY | DWYHPDA | | |
| | | NADLAKTKFA | SPNKLAKKRK | VEQLDAGDV | EEPLRISHY | | |
| | | SRYAYDPQLT | SIIENIRLMLT | LSDETINDLH | GAHKEDVY | | |
| | | QLIYNSIGSIIQ | QWLNTISERE | FVVANLIKG | CYRHPNTG | | |
| | | SPPQEVPKPE | YSNKKELTERF | GLLQRKGS | KDLFTLLQR | | |
| | | GTEENYLLLPN | NADLAKTKFA | (SEQ ID | ADEYVEQL | | |
| | | LKISGASAMN | SRYAYDPQLT | NO: 363) | DAGDVLSD | | |
| | | TPVSIGLPSMT | QLIYNSIGSIIQ | | ETINDLHFV | | |
| | | AFYGFVHAFE | SPPQEVPKPE | | VANLIKGGL | | |
| | | RNLQTVIPNF | GTEENYLLLPN | | LQRKGS | | |
| | | KIESFAVCIHN | LKISGASAMN | | (SEQ ID | | |
| | | LHTENRGLTR | TPVSIGLPSMT | | NO: 364) | | |
| | | EWALNTKDEI | AFYGFVHAFE | | | | |
| | | KAPATRDDW | RNLQTVIPNF | | | | |
| | | QSDLNVSLILQ | KIESFAVCIHN | | | | |
| | | CSNYSQLVPR | LHTENRGLTR | | | | |
| | | DFMYQLPRRL | EWALNTKDEI | | | | |
| | | ARGKVTVAIS | KAPATRDDW | | | | |
| | | AIERLGRSLSL | QSDLNVSLILQ | | | | |
| | | AEAIKTIPVDT | CSNYSQLVPR | | | | |
| | | GRWLSLNSEA | DFMYQLPRRL | | | | |
| | | VLNGIQDIIDE | ARGKVTVAIS | | | | |
| | | LKENRMQTV | AIERLGRSLSL | | | | |
| | | NCIGYHLLELP | AEAIKTIPVDT | | | | |
| | | IEKRCSLRSYK | GRWLSLNSEA | | | | |
| | | HAFAETILGV | VLNGIQDIIDE | | | | |
| | | MKLFAISENT | LKENRMQTV | | | | |
| | | NPDQYFWKY | NCIGYHLLELP | | | | |
| | | HYSKQGPILLP | IEKRCSLRSYK | | | | |
| | | RSLSDEAS | HAFAETILGV | | | | |
| | | (SEQ ID | MKLFAISENT | | | | |
| | | NO: 361) | NPDQYFWKY | | | | |
| | | | HYSKQGPILLP | | | | |
| | | | RSLSDEAS | | | | |
| | | | (SEQ ID | | | | |
| | | | NO: 362) | | | | |
| 21 | 1004634327 | MATLAEILDN | MPKKKRKVGS | MKLPNGLSY | MPKKKRKV | MDWHYRTI | MPKKKRKV |
| | RIMD- | KTDDLNKDLR | GDYKDDDDK | MKSIEASDVI | GSGKLPNG | TFLPEYRNNE | GSGDWHY |
| | BA000032.2 | RAFRPLSAPV | DYKDDDDKD | FLVNWPDG | LSYMKSIEA | AIAAKCIKEL | RTITFLPEYR |
| | | DISDTPIEALTI | YKDDDDKGS | RKTPLPYTSR | SDVIFLVN | HRFNYKYET | NNEAIAAK |
| | | LVNLTDRVIE | GATLAEILDNK | VALGMKEGS | WPDGRKTP | RSIGVSFPLW | CIKELHREN |
| | | QKNLLDRQKC | TDDLNKDLRR | KSAYKYDGQ | LPYTSRVAL | GQETVGRKI | YKYETRSIG |
| | | KDKLRDEKW | AFRPLSAPVDI | IDADVTAYSL | GMKEGSKS | TFVSTNKME | VSFPLWGQ |
| | | WANCFRTVK | SDTPIEALTILV | AQGNPHEID | AYKYDGQI | LDFLISRRYF | ETVGRKITF |
| | | YRQSHNPKFP | NLTDRVIEQK | FCCVPYGAE | DADVTAYS | VQMTKLGYF | VSTNKMEL |
| | | DIRANGVIRA | NLLDRQKCKD | SIECEFSVSFA | LAQGNPHE | SISTTQTVPD | DFLISRRYF |
| | | APVGHLPAC | KLRDEKWWA | SSLRKPFKCS | IDFCCVPYG | DCSYVLFKRA | VQMTKLGY |
| | | MLSSSKLPQN | NCFRTVKYRQ | DPEVKRTLV | AESIECEFS | HSIDKGTFA | FSISTTQTV |
| | | SWAYANDSS | SHNPKFPDIR | QLIKLYEEKV | VSFASSLRK | GRARELKRLE | PDDCSYVLF |
| | | QMNKSCFLTS | ANGVIRAAPV | GWEELANRF | PFKCSDPEV | RRALERGEIF | KRAHSIDKG |
| | | EFIWNGDVH | GHLPACMLSS | LENICNGRW | KRTLVQLIK | DPIAYSKTTS | TFAGRAREL |
| | | CLGQLLTELEH | SKLPQNSWAY | LWRNNECTY | LYEEKVGW | HAFQSYHSL | KRLERRALE |
| | | PLWNVLRKLG | ANDSSQMNK | STSIGIKPWP | EELANRFLE | EEDSSSGNK | RGEIFDPIA |
| | | CYVKTAKYISK | SCFLTSEFIWN | WEDEKAISP | NICNGRWL | FRLNIQMKE | YSKTTSHAF |
| | | ELALIPPLEINT | GDVHCLGQLL | FHDIRKNYA | WRNNECTY | RSGTVGTGK | QSYHSLEED |
| | | SLVRNYLAQIS | TELEHPLWNV | GTNHFRDHK | STSIGIKPW | FSSYGLGNT | SSSGNKFRL |
| | | LPNNEDSYISL | LRKLGCYVKT | DWDNLIKLIT | PWEDEKAI | DNSLQVVPLI | NIQMKERS |
| | | SPVVSQSMQ | AKYISKELALIP | DAFSQPNGL | SPFHDIRKN | (SEQ ID | GTVGTGKF |
| | | EDCYQVLSEH | PLEINTSLVRN | CIFEVSATFR | YAGTNHFR | NO: 371) | SSYGLGNT |
| | | YRFSAITRFSR | YLAQISLPNNE | LGTNAPIYPS | DHKDWDN | | DNSLQVVP |
| | | ATNMGTLAM | DSYISLSPVVS | QVFKDSVKG | LIKLITDAFS | | LI (SEQ ID |
| | | SCGGKFKMIR | QSMQEDCYQ | EKNRIYQSTD | QPNGLCIFE | | NO: 372) |
| | | SLPPIEKYQHH | VLSEHYRFSAI | VDGESSPILG | VSATFRLGT | | |
| | | HLDSVNWLTK | TRFSRATNM | CYKTGAAIAT | NAPIYPSQV | | |
| | | RSVRAIRDYTE | GTLAMSCGG | IDDWYPDAD | FKDSVKGE | | |
| | | SSVWVISPNK | KFKMIRSLPPI | KPIRISHYGA | KNRIYQSTD | | |
| | | LALRKKSIIGDI | EKYQHHHLDS | HREDVYCYR | VDGESSPIL | | |
| | | KMMLSQWLR | VNWLTKRSVR | HPNTGKDLF | GCYKTGAAI | | |
| | | TTPTHEEKLDI | AIRDYTESSV | TLLEKADQYL | ATIDDWYP | | |
| | | RKLTERFNVD | WVISPNKLAL | EQLQATDVL | DADKPIRIS | | |
| | | LAKTKFANRY | RKKSIIGDIKM | PDEMINDLH | HYGAHRED | | |
| | | AYDPLLTQLIY | MLSQWLRTT | FIVANLIKGG | VYCYRHPN | | |
| | | NCIGSIIHSPP | PTHEEKLDIRK | LLQQKGT | TGKDLFTLL | | |
| | | QYAPKCEGN | LTERFNVDLA | (SEQ ID | EKADQYLE | | |
| | | DDKYLLLPNLR | KTKFANRYAY | NO: 369) | QLQATDVL | | |
| | | ISGASAMNTS | DPLLTQLIYNC | | PDEMINDL | | |
| | | VSIGIPSMMA | IGSIIHSPPQY | | HFIVANLIK | | |
| | | FYGFVHAFQR | APKCEGNDDK | | GGLLQQKG | | |
| | | NVQTANPNF | YLLLPNLRISG | | T (SEQ ID | | |
| | | KIESFAVCIHNI | ASAMNTSVSI | | NO: 370) | | |
| | | HVENRGLTRE | GIPSMMAFY | | | | |
| | | WVPNTKGQIT | GFVHAFQRN | | | | |
| | | APATRDDWQ | VQTANPNFKI | | | | |
| | | CDVAVSLILRC | ESFAVCIHNIH | | | | |
| | | SHYSQLIPRDF | VENRGLTRE | | | | |
| | | IRLLPGRIARG | WVPNTKGQIT | | | | |
| | | KVTVSISDIKH | APATRDDWQ | | | | |
| | | LGRCLSLADAI | CDVAVSLILRC | | | | |
| | | KAIPVETGRW | SHYSQLIPRDF | | | | |
| | | LSLNNEVTLNS | IRLLPGRIARG | | | | |
| | | IQDVIDELKN | KVTVSISDIKH | | | | |
| | | NKLQTVNCIG | LGRCLSLADAI | | | | |
| | | YHRLETPCEKR | KAIPVETGRW | | | | |
| | | GSLHGYKHAF | LSLNNEVTLNS | | | | |
| | | VETILGIIKFLTI | IQDVIDELKN | | | | |
| | | SENTNPSQYF | NKLQTVNCIG | | | | |
| | | WQYHYSKQG | YHRLETPCEKR | | | | |
| | | PILLPRSVSDE | GSLHGYKHAF | | | | |
| | | TS (SEQ ID | VETILGIIKFLTI | | | | |
| | | NO: 367) | SENTNPSQYF | | | | |
| | | | WQYHYSKQG | | | | |
| | | | PILLPRSVSDE | | | | |
| | | | TS (SEQ ID | | | | |
| | | | NO: 368) | | | | |
| 22 | V.para_O1 | MATLAEILDN | MPKKKRKVGS | MKLPNNLSY | MPKKKRKV | MDWYYRTIT | MPKKKRKV |
| | Kuk FDA | KTDDLNKDLR | GDYKDDDDK | IKSIEPSDVIF | GSGKLPNN | FLPEYRNNE | GSGDWYYR |
| | R31 | RAFRPLSAPV | DYKDDDDKD | LVNWPDGR | LSYIKSIEPS | AIAAKCIKEL | TITFLPEYR |
| | GCA000430405.1 | DISDTPIEALTI | YKDDDDKGS | KTPLPYTSRV | DVIFLVNW | HRFNYKYET | NNEAIAAK |
| | | LVNLTDRVIE | GATLAEILDNK | ALGMKEGSK | PDGRKTPLP | RSIGVSFPLW | CIKELHRFN |
| | | QKDLLDRKKC | TDDLNKDLRR | SAYKDDGQI | YTSRVALG | GQETVGRKI | YKYETRSIG |
| | | KDKLRDEKW | AFRPLSAPVDI | DMDATAHS | MKEGSKSA | TFVSTNKME | VSFPLWGQ |
| | | WADCFRTVK | SDTPIEALTILV | LAHGNAHEI | YKDDGQID | LDFLISRRYF | ETVGRKITF |
| | | YRQSHNPKFP | NLTDRVIEQK | DFCCVPYGA | MDATAHSL | VQMTKLGYF | VSTNKMEL |
| | | DIRANGVIRA | DLLDRKKCKD | ESIECEFSVSF | AHGNAHEI | SISTTQTVPD | DFLISRRYF |
| | | APVGHLPPF | KLRDEKWWA | ASSLRKPFKC | DFCCVPYG | DCSYVLFKRA | VQMTKLGY |
| | | MLSSSKLPQN | DCFRTVKYRQ | SDPEVKRTLV | AESIECEFS | HSIDKGTSA | FSISTTQTV |
| | | SWAYANDSG | SHNPKFPDIR | QLIKLYEEKV | VSFASSLRK | GRARELKRLE | PDDCSYVLF |
| | | QVNKSCFLTS | ANGVIRAAPV | GWEELANRF | PFKCSDPEV | RRALERGEIF | KRAHSIDKG |
| | | EFIWNGDVLC | GHLPPFMLSS | LENICNGRW | KRTLVQLIK | DPMAYSKTT | TSAGRAREL |
| | | LGQLLTELEHP | SKLPQNSWAY | LWRNNECTY | LYEEKVGW | SHAFQSYHS | KRLERRALE |
| | | LWNVLRKLGC | ANDSGQVNK | STSIGIKPWP | EELANRFLE | LEEDSSSGNK | RGEIFDPM |
| | | YVKTAKYISKE | SCFLTSEFIWN | WEDEKAISP | NICNGRWL | FRLNIQMKE | AYSKTTSHA |
| | | LALIPPLEINTS | GDVLCLGQLL | FHDIRKNYA | WRNNECTY | RSGTVDTGT | FQSYHSLEE |
| | | LVRNYLAQISL | TELEHPLWNV | GTNHFRDHK | STSIGIKPW | FSSYGLGNT | DSSSGNKF |
| | | PNDEDSYISLS | LRKLGCYVKT | DWDKLIKLIT | PWEDEKAI | DNSLQVVPLI | RLNIQMKE |
| | | PVASQSMQE | AKYISKELALIP | DAFSQPNGL | SPFHDIRKN | (SEQ ID | RSGTVDTG |
| | | DCYQVLSEHC | PLEINTSLVRN | CIFEVSATFR | YAGTNHFR | NO: 377) | TFSSYGLGN |
| | | RFSAITRFSRA | YLAQISLPNDE | LGTNAPIYPS | DHKDWDK | | TDNSLQVV |
| | | TNMGTLAMS | DSYISLSPVAS | QVFKDSVKG | LIKLITDAFS | | PLI (SEQ ID |
| | | CGGKFKMIRS | QSMQEDCYQ | EKNRIYQSTN | QPNGLCIFE | | NO: 378) |
| | | LPPIEKYQHH | VLSEHCRFSAI | VDGESSPILG | VSATFRLGT | | |
| | | HLDSVNWLTK | TRFSRATNM | CYKTGAAIAT | NAPIYPSQV | | |
| | | RSVRAIRDYTE | GTLAMSCGG | IDDWYPDAD | FKDSVKGE | | |
| | | SSVWVISPNK | KFKMIRSLPPI | KPIRISHYGA | KNRIYQSTN | | |
| | | LALRKKSIIEDI | EKYQHHHLDS | HKEDVYCYR | VDGESSPIL | | |
| | | KIMLSQWLRT | VNWLTKRSVR | HPNTGKDLF | GCYKTGAAI | | |
| | | TPTHEEKLDIR | AIRDYTESSV | TLLEKADQYL | ATIDDWYP | | |
| | | KLTERFNVDL | WVISPNKLAL | EQLQATEVL | DADKPIRIS | | |
| | | AKTEFANRYA | RKKSIIEDIKIM | PDEMINDLH | HYGAHKED | | |
| | | YDPLLTQLIYN | LSQWLRTTPT | FIVANLIKGG | VYCYRHPN | | |
| | | CIGSIIHSPPQ | HEEKLDIRKLT | LLQRKGT | TGKDLFTLL | | |
| | | DAPKCEGND | ERFNVDLAKT | (SEQ ID | EKADQYLE | | |
| | | DKYLLLPNLRI | EFANRYAYDP | NO: 375) | QLQATEVL | | |
| | | SGASAMNTS | LLTQLIYNCIG | | PDEMINDL | | |
| | | VSIGIPSMMA | SIIHSPPQDAP | | HFIVANLIK | | |
| | | FYGFVHAFQR | KCEGNDDKYL | | GGLLQRKG | | |
| | | NVQTANPNF | LLPNLRISGAS | | T (SEQ ID | | |
| | | KIESFAVCIHNI | AMNTSVSIGI | | NO: 376) | | |
| | | HVENRGLTRE | PSMMAFYGF | | | | |
| | | WVPNTKGQIT | VHAFQRNVQ | | | | |
| | | APATRDDWQ | TANPNFKIESF | | | | |
| | | CDVAVSLILRC | AVCIHNIHVE | | | | |
| | | SHYSQLIPRDF | NRGLTREWV | | | | |
| | | IRLLPGRIARG | PNTKGQITAP | | | | |
| | | KVTVSISDIKH | ATRDDWQCD | | | | |
| | | LGRCLSLADAI | VAVSLILRCSH | | | | |
| | | KAIPVETGRW | YSQLIPRDFIRL | | | | |
| | | LSLNNEVTLNS | LPGRIARGKV | | | | |
| | | IQDVIDELKN | TVSISDIKHLG | | | | |
| | | NRLQTVSCIG | RCLSLADAIKA | | | | |
| | | YQLLEPPCEKR | IPVETGRWLS | | | | |
| | | GSLHGYKHAF | LNNEVTLNSI | | | | |
| | | VETILGIIKLLA | QDVIDELKNN | | | | |
| | | SKNTNPDQYR | RLQTVSCIGY | | | | |
| | | WQYHYSKQG | QLLEPPCEKR | | | | |
| | | PILLLKSISDET | GSLHGYKHAF | | | | |
| | | S (SEQ ID | VETILGIIKLLAI | | | | |
| | | NO: 373) | SKNTNPDQYF | | | | |
| | | | WQYHYSKQG | | | | |
| | | | PILLLKSISDET | | | | |
| | | | S (SEQ ID | | | | |
| | | | NO: 374) | | | | |
| 23 | V.fisc.MJ11 | MEFTDILIIQD | MPKKKRKVGS | MKLCNNLNY | MPKKKRKV | MLTHYFSITY | MPKKKRKV |
| | GCA000020845.1 | VKERNRALKV | GDYKDDDDK | TRSLSPGKAV | GSGKLCNN | VPDDCDNEL | GSGLTHYFS |
| | | AFAHYSSAICI | DYKDDDDKD | FYYESKDGQ | LNYTRSLSP | LAGRCIAEFH | ITYVPDDCD |
| | | DEHEVEAITCL | YKDDDDKGS | MNPIKCEQT | GKAVFYYES | KFISSLRLIEN | NELLAGRCI |
| | | LNLCTPKTEDY | GEFTDILIIQD | HLRAPKAGF | KDGQMNPI | NSFAIGFPN | AEFHKFISSL |
| | | LDKTSASLFLN | VKERNRALKV | SEAFNSDYST | KCEQTHLR | WSEQSVGN | RLIENNSFAI |
| | | NHDNIQKCLD | AFAHYSSAICI | KNTAPQDLS | APKAGFSE | EFAIFSDNSE | GFPNWSEQ |
| | | ELKWFHSHN | DEHEVEAITCL | FSNPQFIEEC | AFNSDYSTK | LLSAIKYQPY | SVGNEFAIF |
| | | VKYPDCRVKG | LNLCTPKTEDY | YVPVGIDEIKI | NTAPQDLS | FNLMRNEEL | SDNSELLSA |
| | | QSIISLPIDSVS | LDKTSASLFLN | RFSLRIEANS | FSNPQFIEE | FSITDIKPVP | IKYQPYFNL |
| | | NTINSNVVPY | NHDNIQKCLD | LQPDKCSDV | CYVPVGIDE | NNLPQIRFIR | MRNEELFSI |
| | | RLGWSHDSG | ELKWFHSHN | QIREILQAFA | IKIRFSLRIEA | NQSIGKIFIG | TDIKPVPNN |
| | | KVNYTHFLLSQ | VKYPDCRVKG | TKYKENGGY | NSLQPDKC | SKKRRIQRSI | PQIRFIRN |
| | | FKWRGVQTT | QSIISLPIDSVS | QELGERYAK | SDVQIREIL | TRNNKEHTPI | QSIGKIFIGS |
| | | LSQLFITDTLF | NTINSNVVPY | NLLSGTWL | QAFATKYK | SNEDREFDT | KKRRIQRSI |
| | | WLDIIKKIQCN | RLGWSHDSG | WRNEHNLG | ENGGYQEL | FHKVSCSSKS | TRNNKEHT |
| | | WTKKQTEQFI | KVNYTHFLLSC | TSISIKTTSNQ | GERYAKNLL | KQQQYILHI | PISNEDREF |
| | | HSIQKEMPAK | FKWRGVQTT | EFNIDNAFKL | SGTWLWR | QKDITPRTID | DTFHKVSCS |
| | | TLPENISPYSK | LSQLFITDTLF | SRKTSAKDK | NEHNLGTSI | SKGSYNSYGL | SKSKQQQYI |
| | | QILFPYKNDYL | WLDIIKKIQCN | KTISKLGSEIA | SIKTTSNQE | ATNSKHLGT | LHIQKDITP |
| | | TLTPVTSNSV | WTKKQTEQFI | SALSDPDHY | FNIDNAFKL | VPDLSKIPFY | RTIDSKGSY |
| | | QTWLEHQSR | HSIQKEMPAK | YFADITATIN | SRKTSAKDK | CEEKLSNKD | NSYGLATN |
| | | KPDDIRWIKR | TLPENISPYSK | VAFCQEIYPS | KTISKLGSEI | Q (SEQ ID | SKHLGTVP |
| | | ESKHSASVGA | QILFPYKNDYL | QEFLDTKEK | ASALSDPD | NO: 383) | DLSKIPFYCE |
| | | LSSSIGGYHSL | TLTPVTSNSV | GKPSKVYAK | HYYFADITA | | EKLSNKDQ |
| | | LFSPPSTSQSP | QTWLEHQSR | TSLLTDEKTV | TINVAFCQE | | (SEQ ID |
| | | HSYHDNMAS | KPDDIRWIKR | ALHAQKIGA | IYPSQEFLD | | NO: 384) |
| | | KTGCREAFCT | ESKHSASVGA | AIQLIDDWW | TKEKGKPSK | | |
| | | SAITEKSTTDA | LSSSIGGYHSL | ADDADIPLR | VYAKTSLLT | | |
| | | LQRLISSEVR | LFSPPSTSQSP | VNEFGADH | DEKTVALH | | |
| | | MNVKHRKKIR | HSYHDNMAS | HNVIARRHP | AQKIGAAIQ | | |
| | | KSGVHFIRQKI | KTGCREAFCT | SHRNDFYTLI | LIDDWWA | | |
| | | ALWLTPLIRW | SAITEKSTTDA | QNADNYCA | DDADIPLRV | | |
| | | RDHIDNNQIQ | LQRLISSEVR | QLDENSDIT | NEFGADHH | | |
| | | ITNDHPSLVNL | MNVKHRKKIR | DDMHYVMA | NVIARRHPS | | |
| | | FLSSPIASFPDL | KSGVHFIRQKI | VLVKGGLFQ | HRNDFYTLI | | |
| | | LAPLHNHLNQ | ALWLTPLIRW | KSASSKKGK | QNADNYCA | | |
| | | TLGKNKYTKR | RDHIDNNQIQ | (SEQ ID | QLDENSDIT | | |
| | | FAYHPDLMPI | ITNDHPSLVNL | NO: 381) | DDMHYVM | | |
| | | FKSQLSWILN | FLSSPIASFPDL | | AVLVKGGL | | |
| | | KLAQDENINQ | LAPLHNHLNQ | | FQKSASSKK | | |
| | | QPVLPRTQFI | TLGKNKYTKR | | GK (SEQ ID | | |
| | | HLKNLRLYNG | FAYHPDLMPI | | NO: 382) | | |
| | | NALSSPYVCG | FKSQLSWILN | | | | |
| | | LPSLTGFWGF | KLAQDENINQ | | | | |
| | | MHDFERRLKT | QPVLPRTQFI | | | | |
| | | KIEENIHFEAF | HLKNLRLYNG | | | | |
| | | SLFVHQYELQ | NALSSPYVCG | | | | |
| | | SSPPLCEASDI | LPSLTGFWGF | | | | |
| | | YKKRELSPAKR | MHDFERRLKT | | | | |
| | | LLTQPSYSCD | KIEENIHFEAF | | | | |
| | | MRFDLIIKVHT | SLFVHQYELQ | | | | |
| | | EVNLSDISQR | SSPPLCEASDI | | | | |
| | | MLSAMPARC | YKKRELSPAKR | | | | |
| | | VGGTLHQSSL | LLTQPSYSCD | | | | |
| | | HESLEWLTSY | MRFDLIIKVHT | | | | |
| | | ASSEHLYEELA | EVNLSDISQR | | | | |
| | | CLPNSGRWIY | MLSAMPARC | | | | |
| | | PPSETFNTPD | VGGTLHQSSL | | | | |
| | | EFLSILGNSTH | HESLEWLTSY | | | | |
| | | LAICNGYSFLE | ASSEHLYEELA | | | | |
| | | DPTNRENVSL | CLPNSGRWIY | | | | |
| | | NQHVFCEPLI | PPSETFNTPD | | | | |
| | | GLAEQVIPID | EFLSILGNSTH | | | | |
| | | MRLNRQKYYF | LAICNGYSFLE | | | | |
| | | SNAFWSINSD | DPTNRENVSL | | | | |
| | | FNSILIQKHE | NQHVFCEPLI | | | | |
| | | (SEQ ID | GLAEQVIPID | | | | |
| | | NO: 379) | MRLNRQKYYF | | | | |
| | | | SNAFWSINSD | | | | |
| | | | FNSILIQKHE | | | | |
| | | | (SEQ ID | | | | |
| | | | NO: 380) | | | | |
| 24 | V.paraISF- | MTLDELLAAT | MPKKKRKVGS | MKLPIHLAYE | MPKKKRKV | MMLYYRTVT | MPKKKRKV |
| | 25-6 | DLEELVSSTKR | GDYKDDDDK | RSISPSDVAF | GSGKLPIHL | FLPKIKNNEA | GSGMLYYR |
| | | AFRPLSPLIDIT | DYKDDDDKD | LVVWPDGN | AYERSISPS | LIGHCLKVLH | TVTFLPKIK |
| | | QNPLNALTILI | YKDDDDKGS | KKPLPCYSRT | DVAFLVVW | GVCTKYTINT | NNEALIGH |
| | | NLTEKGISNK | GTLDELLAAT | ILGLNEGSHV | PDGNKKPL | IGVSFPEWG | CLKVLHGV |
| | | NLLDRTRCKE | DLEELVSSTKR | GYDDSGTVR | PCYSRTILGL | KESIGDKISFI | CTKYTINTI |
| | | KLRDDKWWA | AFRPLSPLIDIT | NNLKMNTLV | NEGSHVGY | SPKPLELDFL | GVSFPEWG |
| | | AVLKPAQYRH | QNPLNALTILI | DGNIHELDY | DDSGTVRN | LQQNYFAE | KESIGDKISF |
| | | SHNVKFPDIRS | NLTEKGISNK | CSVPYGAKSI | NLKMNTLV | MTALGYFSIS | ISPKPLELDF |
| | | TGTIRTIAPDN | NLLDRTRCKE | ECCFSVSFSS | DGNIHELD | ESTTVPEECN | LLQQNYFA |
| | | LPAYFITSSKLP | KLRDDKWWA | ELLKPYKCSD | YCSVPYGA | LAVFRRNQKI | EMTALGYF |
| | | NVGWTYSKD | AVLKPAQYRH | ADVKKTLREF | KSIECCFSVS | DQATPNGQ | SISESTTVPE |
| | | SSDINRCLFFT | SHNVKFPDIRS | INLYNQRVEL | FSSELLKPYK | RIRAERLAKR | ECNLAVFR |
| | | SEFLWAGQA | TGTIRTIAPDN | DELIIKYLTNI | CSDADVKK | AMNRGDSPI | RNQKIDQA |
| | | CCLAKTLTDSE | LPAYFITSSKLP | ALGTWLWH | TLREFINLY | RFIPKDHVFE | TPNGQRIR |
| | | HPLWSTLKK | NVGWTYSKD | NTKRSYCVSI | NQRVELDE | HYHSIPITST | AERLAKRA |
| | | MGCYEKHKN | SSDINRCLFFT | EVRPWPWE | LIIKYLTNIAL | QSGKSFRLN | MNRGDSPI |
| | | LAVKLLSQIPD | SEFLWAGQA | GEPIIIDDIRK | GTWLWHN | LQYQQLGTV | RFIPKDHVF |
| | | ELIDVDLSGNY | CCLAKTLTDSE | YLKGESDTN | TKRSYCVSI | TDGEWAFSS | EHYHSIPITS |
| | | LSQVSFPDGH | HPLWSTLKK | DLLNWKKLI | EVRPWPW | YGLANQKLK | TQSGKSFRL |
| | | DSYLSFSPVAS | MGCYEKHKN | KQVKEAFTD | EGEPIIIDDI | SSPVPVI | NLQYQQLG |
| | | QAMQSCVYQ | LAVKLLSQIPD | PMGLCILEVK | RKYLKGESD | (SEQ ID | TVTDGEWA |
| | | SLEQHYRQTA | ELIDVDLSGNY | ANLIKPSMA | TNDLLNWK | NO: 389) | FSSYGLAN |
| | | LMGFDRATN | LSQVSFPDGH | QLYPSQMFK | KLIKQVKEA | | QKLKSSPVP |
| | | MGLLAASCG | DSYLSFSPVAS | EAAKKENNR | FTDPMGLC | | VI (SEQ ID |
| | | GRFRLIETKTYI | QAMQSCVYQ | LYQSTIIDGIK | ILEVKANLIK | | NO: 390) |
| | | KDKRHHYISE | SLEQHYRQTA | SPIMGCYKT | PSMAQLYP | | |
| | | QPNWLTKEAI | LMGFDRATN | GAAIAKIDT | SQMFKEAA | | |
| | | QSIEQFLSSEQ | MGLLAASCG | WYPDAEEPI | KKENNRLY | | |
| | | WLVTHNDKP | GRFRLIETKTYI | RVGHYGVDR | QSTIIDGIKS | | |
| | | RNMAIVKSSI | KDKRHHYISE | ENSTAYRHP | PIMGCYKT | | |
| | | RTMVNRWLS | QPNWLTKEAI | STGKDFFSIL | GAAIAKIDT | | |
| | | TRTITEDLSPA | QSIEQFLSSEQ | KRTDEFVDR | WYPDAEEP | | |
| | | ALTEQLNAD | WLVTHNDKP | LKDSEELNQ | IRVGHYGV | | |
| | | MASIRIIKRYA | RNMAIVKSSI | DNLNDMHF | DRENSTAY | | |
| | | YQPKLTRLFIQ | RTMVNRWLS | LMANLIKGG | RHPSTGKD | | |
| | | LIESAVEDNDY | TRTITEDLSPA | LFQEKGE | FFSILKRTDE | | |
| | | KEDREATTNS | ALTEQLNAD | (SEQ ID | FVDRLKDSE | | |
| | | QYLLIPELRISG | MASIRIIKRYA | NO: 387) | ELNQDNLN | | |
| | | GSAKSSSASV | YQPKLTRLFIQ | | DMHFLMA | | |
| | | GLFSMMSLY | LIESAVEDNDY | | NLIKGGLFQ | | |
| | | GFIHAFERNM | KEDREATTNS | | EKGE (SEQ | | |
| | | RHVLTNFTINS | QYLLIPELRISG | | ID NO: 388) | | |
| | | FAICIHDYHLE | GSAKSSSASV | | | | |
| | | KRGLTKEPIKK | GLFSMMSLY | | | | |
| | | AKVSRDEKEKI | GFIHAFERNM | | | | |
| | | APPAIYDDYQ | RHVLTNFTINS | | | | |
| | | FDSCISLIIKTSE | FAICIHDYHLE | | | | |
| | | SKTIPAEKIVAL | KRGLTKEPIKK | | | | |
| | | LPKRFARGSIR | AKVSRDEKEKI | | | | |
| | | LFIDGIKNIAPF | APPAIYDDYQ | | | | |
| | | PEPLPAIQAIN | FDSCISLIIKTSE | | | | |
| | | NPHGSWLSFE | SKTIPAEKIVAL | | | | |
| | | PDLSLTSTDSL | LPKRFARGSIR | | | | |
| | | VDITINRSNLL | LFIDGIKNIAPF | | | | |
| | | LTVMGYQYLE | PEPLPAIQAIN | | | | |
| | | PPTTKPGSLR | NPHGSWLSFE | | | | |
| | | DYPHALVENIL | PDLSLTSTDSL | | | | |
| | | GFVKPRTVTQ | VDITINRSNLL | | | | |
| | | STNLDDLFWR | LTVMGYQYLE | | | | |
| | | YQVTHFGVCL | PPTTKPGSLR | | | | |
| | | LPRSIK (SEQ | DYPHALVENIL | | | | |
| | | ID NO: 385) | GFVKPRTVTQ | | | | |
| | | | STNLDDLFWR | | | | |
| | | | YQVTHFGVCL | | | | |
| | | | LPRSIK (SEQ | | | | |
| | | | ID NO: 386) | | | | |
| 25 | V.cholerae_ | MTKLSDLLVIE | MPKKKRKVGS | MELCTQLNY | MPKKKRKV | MSQRYYFLIR | MPKKKRKV |
| | YB2A06_ | DEAIKQTALKK | GDYKDDDDK | VRSLSAGKA | GSGELCTQL | YTNANADYG | GSGSQRYY |
| | GCA_ | MFMPYTEDV | DYKDDDDKD | YFYYLSESGE | NYVRSLSA | LLAGRCISQT | FLIRYTNAN |
| | 001402375.1 | CVDGYEQETL | YKDDDDKGS | MCPLNVDKT | GKAYFYYLS | HLFMVNNH | ADYGLLAG |
| | | TILLNLSSSHQ | GTKLSDLLVIE | RLRAPKGSYS | ESGEMCPL | QAMNRVGV | RCISQTHLF |
| | | ADRCSDWLD | DEAIKQTALKK | EAYKGNKFV | NVDKTRLR | SFPDWNESS | MVNNHQA |
| | | VARAQRYLKD | MFMPYTEDV | DKNVAPQDL | APKGSYSEA | VGQTIAFVSE | MNRVGVSF |
| | | RENLDASLAEI | CVDGYEQETL | AYSNPQFIEE | YKGNKFVD | DKEMMIGLS | PDWNESSV |
| | | QWFHTHNLK | TILLNLSSSHQ | CYVKPGVDEI | KNVAPQDL | FQPYFSLMV | GQTIAFVSE |
| | | FPDCRVKDQR | ADRCSDWLD | YCAFSLRIRA | AYSNPQFIE | KEGLFELSSIC | DKEMMIGL |
| | | IIARPLSTAEEF | VARAQRYLKD | NSLTPDICSD | ECYVKPGV | EVPDNLGEV | SFQPYFSL |
| | | ISSAVLDQRLG | RENLDASLAEI | DEVRSKLSM | DEIYCAFSL | RFVRNQTIN | MVKEGLFE |
| | | WAHNSAVYR | QWFHTHNLK | FSKIYKELNG | RIRANSLTP | KSFLGSKKRR | LSSICEVPD |
| | | HTLWLLNPFK | FPDCRVKDQR | YKELANRYA | DICSDDEVR | IKRSMVRAE | NLGEVRFV |
| | | WQSQPVCILS | IIARPLSTAEEF | KNILLGTWL | SKLSMFSKI | LSGAEQRLP | RNQTINKSF |
| | | LIQQKNPVWL | ISSAVLDQRLG | WRNRECRNI | YKELNGYKE | VTNEDRVID | LGSKKRRIK |
| | | DLLTEFGLDVK | WAHNSAVYR | TIEVTTSELD | LANRYAKNI | SFHRIPISSGS | RSMVRAEL |
| | | SLARLQRAIEE | HTLWLLNPFK | TFVVEHAQK | LLGTWLWR | SRQDFILFIQ | SGAEQRLP |
| | | QLPENSFPNS | WQSQPVCILS | LSWYGHWD | NRECRNITI | KELADERAES | VTNEDRVI |
| | | VSAYSKQLRF | LIQQKNPVWL | GDSTECLERL | EVTTSELDT | GFNSYALAT | DSFHRIPISS |
| | | PWGDDYVSIT | DLLTEFGLDVK | TAYLERALSD | FVVEHAQK | NQERRGTVP | GSSRQDFIL |
| | | PVVSHALQCE | SLARLQRAIEE | PTEYFYMDV | LSWYGHW | DLRF (SEQ | FIQKELADE |
| | | LEIRARSPENK | QLPENSFPNS | KAKMRVGW | DGDSTECLE | ID NO: 395) | RAESGFNSY |
| | | FSFVSSSLPNS | VSAYSKQLRF | GDEVYPSQE | RLTAYLERA | | ALATNQER |
| | | ASIGNLCGSLG | PWGDDYVSIT | FLDSREDGIP | LSDPTEYFY | | RGTVPDLR |
| | | GYMRVLNYPL | PVVSHALQCE | TKQLATVELL | MDVKAKM | | F (SEQ ID |
| | | GVKQAKGGTL | LEIRARSPENK | RGKETVAFH | RVGWGDE | | NO: 396) |
| | | TGNRQKSGH | FSFVSSSLPNS | GQKVGAAL | VYPSQEFLD | | |
| | | YFDDYQVTNA | ASIGNLCGSLG | QSIDDWWH | SREDGIPTK | | |
| | | KICQVLNRLIG | GYMRVLNYPL | EEADKPLRV | QLATVELLR | | |
| | | SEPSKTQRQR | GVKQAKGGTL | NEYGADREY | GKETVAFH | | |
| | | ERARQVRGKI | TGNRQKSGH | VIARRHVTH | GQKVGAAL | | |
| | | LRKQIALWML | YFDDYQVTNA | GNDFYQLVR | QSIDDWW | | |
| | | PLIELRDIAESE | KICQVLNRLIG | NTENWIEA | HEEADKPL | | |
| | | PNQQQLEHD | SEPSKTQRQR | MTASQTIPN | RVNEYGAD | | |
| | | DTLAQAFLSLP | ERARQVRGKI | DVHFIMSVLI | REYVIARRH | | |
| | | ELELGSLAGEF | LRKQIALWML | KGGLFNCAK | VTHGNDFY | | |
| | | NRRLHLTFQN | PLIELRDIAESE | AN (SEQ ID | QLVRNTEN | | |
| | | NIYSAKFAYHP | PNQQQLEHD | NO: 393) | WIEAMTAS | | |
| | | KLMQVAKAQ | DTLAQAFLSLP | | QTIPNDVH | | |
| | | VTWVLEQLSK | ELELGSLAGEF | | FIMSVLIKG | | |
| | | PINNQDKVTG | NRRLHLTFQN | | GLFNCAKA | | |
| | | EQYIYLSSMR | NIYSAKFAYHP | | N (SEQ ID | | |
| | | VQDAVAMSN | KLMQVAKAQ | | NO: 394) | | |
| | | PCLCGVPSLTA | VTWVLEQLSK | | | | |
| | | IWGVMHDYQ | PINNQDKVTG | | | | |
| | | RKFNQLVNN | EQYIYLSSMR | | | | |
| | | GSPVEFSSFAF | VQDAVAMSN | | | | |
| | | YVRNENIQST | PCLCGVPSLTA | | | | |
| | | AKLTEPNSVA | IWGVMHDYQ | | | | |
| | | KARTVSNAKR | RKFNQLVNN | | | | |
| | | PTIRSERLSDL | GSPVEFSSFAF | | | | |
| | | EIDLVIRVHSE | YVRNENIQST | | | | |
| | | SRISDFRSALK | AKLTEPNSVA | | | | |
| | | TALPVAFAGG | KARTVSNAKR | | | | |
| | | ALYQPHLSTQI | PTIRSERLSDL | | | | |
| | | EWLRTFTGRS | EIDLVIRVHSE | | | | |
| | | ELFHVLKGLPA | SRISDFRSALK | | | | |
| | | YGRWLYPSEK | TALPVAFAGG | | | | |
| | | QPTNFDELER | ALYQPHLSTQI | | | | |
| | | LLTQDDDNLP | EWLRTFTGRS | | | | |
| | | VSLGYHLLEHP | ELFHVLKGLPA | | | | |
| | | TKRDNAITGC | YGRWLYPSEK | | | | |
| | | HAYAENAIGL | QPTNFDELER | | | | |
| | | AKRINPIEVRF | LLTQDDDNLP | | | | |
| | | SGRDHFLNHA | VSLGYHLLEHP | | | | |
| | | FWSIECSSETIL | TKRDNAITGC | | | | |
| | | IKNYRD (SEQ | HAYAENAIGL | | | | |
| | | ID NO: 391) | AKRINPIEVRF | | | | |
| | | | SGRDHFLNHA | | | | |
| | | | FWSIECSSETIL | | | | |
| | | | IKNYRD (SEQ | | | | |
| | | | ID NO: 392) | | | | |
| 26 | Agarivorans | MTLADIITTQ | MPKKKRKVGS | MQLCKQLKY | MPKKKRKV | VIERYYFIVRY | MPKKKRKV |
| | gilvus | NIAERNRALK | GDYKDDDDK | ERSIQPGKA | GSGQLCKQ | LPKRADCSLL | GSGIERYYFI |
| | strain | RAFAPDSNGV | DYKDDDDKD | VFFYKTEDSE | LKYERSIQP | AGRCIKELHH | VRYLPKRA |
| | WH0801 | EVVGKEQEAL | YKDDDDKGS | FVPLEADIKRI | GKAVFFYKT | IFSQTEESIAV | DCSLLAGRC |
| | | VVLLNLSLRKE | GTLADIITTQN | RGQKTSFSE | EDSEFVPLE | SFPEWTVGS | IKELHHIFS |
| | | EVDDLCDQTL | IAERNRALKR | AYASIAKPKN | ADIKRIRGQ | LGPSIGFVSS | QTEESIAVS |
| | | ATTTLRNQKH | AFAPDSNGVE | VAVQDLAYS | KTSFSEAYA | SVKYLEALRN | FPEWTVGS |
| | | LQLCCSEIQW | VVGKEQEALV | NPIRMETVT | SIAKPKNVA | RSYFIDMQEI | LGPSIGFVS |
| | | LHSHNLKFPN | VLLNLSLRKEE | VPPLVEAIYC | VQDLAYSN | GAFELTKVLT | SSVKYLEAL |
| | | ARVSHQRLLT | VDDLCDQTLA | RFNLRIFANS | PIRMETVTV | VPNEVGEVR | RNRSYFID |
| | | SPQVPVSGTL | TTTLRNQKHL | LEPSVCDDL | PPLVEAIYC | FIRNQRVAKL | MQEIGAFE |
| | | SSANFPVRYG | QLCCSEIQWL | DTHNILKQL | RFNLRIFAN | FSGEFRRRYA | LTKVLTVPN |
| | | WSHDSARIRK | HSHNLKFPNA | ANGYRQKEG | SLEPSVCDD | RGKKRPKLG | EVGEVRFIR |
| | | ASLFCAEFKW | RVSHQRLLTS | YKELAKRYAK | LDTHNILKQ | GKALIRNTC | NQRVAKLF |
| | | NGLWTCLAKE | PQVPVSGTLS | NLLLGQWLF | LANGYRQK | QRMLRSPHS | SGEFRRRYA |
| | | LDERDHIWQK | SANFPVRYG | RNQQTYPVS | EGYKELAKR | IRLLYRVVQV | RGKKRPKL |
| | | VFFELGFSRRD | WSHDSARIRK | IELLTSNNSIF | YAKNLLLG | SSISFFIYKKS | GGKALIRNT |
| | | FQALTAMVG | ASLFCAEFKW | SVNDVHQF | QWLFRNQ | LPKLLKPQGF | CQRMLRSP |
| | | ELLGEETFPQE | NGLWTCLAKE | DWNSRSNSY | QTYPVSIEL | VVTALLRHA | HSIRLLYRV |
| | | VSPFSSQIRVP | LDERDHIWQK | INQVEKLAAE | LTSNNSIFS | RKGELFQT* | VQVSSISFFI |
| | | FKNSYCSVTP | VFFELGFSRRD | LAGAFSEPRR | VNDVHQFD | (SEQ ID | YKKSLPKLL |
| | | VVSHSLQSAI | FQALTAMVG | YWSAEVTAK | WNSRSNSY | NO: 401) | KPQGFVVT |
| | | QNLDYILKKG | ELLGEETFPQE | ISAQMGEEIF | INQVEKLAA | | ALLRHARK |
| | | KFKRLQHEHS | VSPFSSQIRVP | PSQQLTEKV | ELAGAFSEP | | GELFQT* |
| | | ASIGNLCAAH | FKNSYCSVTP | EKGEISKLFC | RRYWSAEV | | (SEQ ID |
| | | GGRVSSLFYP | VVSHSLQSAI | KLAMPDGRE | TAKISAQM | | NO: 402) |
| | | PHIIKYQHVTL | QNLDYILKKG | AVILNMEKV | GEEIFPSQQ | | |
| | | SSSLEKRSKSD | KFKRLQHEHS | GAGIQMIDD | LTEKVEKGE | | |
| | | SVFNRKAINN | ASIGNLCAAH | WYTDEADYR | ISKLFCKLA | | |
| | | KIFHNALRALI | GGRVSSLFYP | LRVHEYGAD | MPDGREA | | |
| | | NPSVEITLKKR | PHIIKYQHVTL | PKHVIAQRR | VILNMEKV | | |
| | | RQRRLSALRY | SSSLEKRSKSD | PETHSDFYSL | GAGIQMID | | |
| | | VRKELAAWLA | SVFNRKAINN | VSQAEAHLE | DWYTDEA | | |
| | | PVMEWRDSL | KIFHNALRALI | VLKQAVSSS | DYRLRVHE | | |
| | | EETEGTLNELE | NPSVEITLKKR | DIPAEIHYV | YGADPKHV | | |
| | | QDSLVYRLLTF | RQRRLSALRY | MSVLIKGGM | IAQRRPETH | | |
| | | EPCDFPVLLN | VRKELAAWLA | FQRGKEG | SDFYSLVSQ | | |
| | | QLNICLHEELQ | PVMEWRDSL | (SEQ ID | AEAHLEVLK | | |
| | | TSFYGAEFAF | EETEGTLNELE | NO: 399) | QAVSSSDIP | | |
| | | HPRLIHPLKSQ | QDSLVYRLLTF | | AEIHYVMS | | |
| | | LLWLLNYLGK | EPCDFPVLLN | | VLIKGGMF | | |
| | | DDDESDVESD | QLNICLHEELQ | | QRGKEG | | |
| | | VQYIYFSNLRV | TSFYGAEFAF | | (SEQ ID | | |
| | | FDADAMANP | HPRLIHPLKSQ | | NO: 400) | | |
| | | YLCGIPSLTAV | LLWLLNYLGK | | | | |
| | | WGMCHRFQL | DDDESDVESD | | | | |
| | | QLNKLLPESVS | VQYIYFSNLRV | | | | |
| | | VDGFTWFVH | FDADAMANP | | | | |
| | | QYSLSAGRKL | YLCGIPSLTAV | | | | |
| | | PEPSRYIRNEL | WGMCHRFQL | | | | |
| | | KRPGFIAGQH | QLNKLLPESVS | | | | |
| | | CDLTIDLILKIS | VDGFTWFVH | | | | |
| | | AREDFRLSDD | QYSLSAGRKL | | | | |
| | | DIPLIQASLPA | PEPSRYIRNEL | | | | |
| | | KLAGGSVHPP | KRPGFIAGQH | | | | |
| | | SLYERREWCS | CDLTIDLILKIS | | | | |
| | | LYSVQHELFD | AREDFRLSDD | | | | |
| | | RLARLPTGGR | DIPLIQASLPA | | | | |
| | | WVFPTHQEV | KLAGGSVHPP | | | | |
| | | HSLEELMDIIT | SLYERREWCS | | | | |
| | | SDYSIKPAML | LYSVQHELFD | | | | |
| | | GYLLLEEPTLR | RLARLPTGGR | | | | |
| | | EGALTSMHAY | WVFPTHQEV | | | | |
| | | AEPLLGLVQTL | HSLEELMDIIT | | | | |
| | | SAIDVRIMKP | SDYSIKPAML | | | | |
| | | KVFWAAAFW | GYLLLEEPTLR | | | | |
| | | QLKVSERAML | EGALTSMHAY | | | | |
| | | MKSL (SEQ ID | AEPLLGLVQTL | | | | |
| | | NO: 397) | SAIDVRIMKP | | | | |
| | | | KVFWAAAFW | | | | |
| | | | QLKVSERAML | | | | |
| | | | MKSL (SEQ ID | | | | |
| | | | NO: 398) | | | | |
| 27 | V.cholerae_ | MTKLSDLLAIE | MPKKKRKVGS | MELCTQLNY | MPKKKRKV | MSQRYYFLIR | MPKKKRKV |
| | VC35_GCA_ | DEAIKQTALKK | GDYKDDDDK | VRSLSAGKA | GSGELCTQL | YTNANADYG | GSGSQRYY |
| | 000299495.2 | MFMPYTEDV | DYKDDDDKD | YFYYLSESGE | NYVRSLSA | LLAGRCISQ | FLIRYTNAN |
| | | CVDGYEQETL | YKDDDDKGS | MCPLDVDRT | GKAYFYYLS | MHLFMVNH | ADYGLLAG |
| | | TILLNLSSSHQ | GTKLSDLLAIE | RLRAPKGSYS | ESGEMCPL | HQAMNRVG | RCISQMHL |
| | | ADRCSDWLD | DEAIKQTALKK | EAYKGNKFV | DVDRTRLR | VSFPDWNES | FMVNHHQ |
| | | VARAQRYLKD | MFMPYTEDV | DKNVAPQDL | APKGSYSEA | SVGQTIAFVS | AMNRVGV |
| | | RENLDASLAEI | CVDGYEQETL | AYSNPQFIEE | YKGNKFVD | EDKEMMIGL | SFPDWNES |
| | | QWFHTHNLK | TILLNLSSSHQ | CYVKPGVDEI | KNVAPQDL | SFQPYFSLM | SVGQTIAFV |
| | | FPDCRVKDQR | ADRCSDWLD | YCAFSLRIRA | AYSNPQFIE | VNEGLFEISS | SEDKEMMI |
| | | IIARPLSTAEEF | VARAQRYLKD | NSLTPDMCS | ECYVKPGV | VYEVPDTSA | GLSFQPYFS |
| | | ISSAVLDQRLG | RENLDASLAEI | DDEVRSKLS | DEIYCAFSL | EVRFVRNQT | LMVNEGLF |
| | | WAHNSAVYR | QWFHTHNLK | MLAKIYKDL | RIRANSLTP | IGKNFLGSKK | EISSVYEVP |
| | | HTLWLLNPFK | FPDCRVKDQR | NGYKELAHR | DMCSDDEV | RRIKRSMAR | DTSAEVRFV |
| | | WQSQPVCILL | IIARPLSTAEEF | YAKNILLGT | RSKLSMLA | AELFGVEQSL | RNQTIGKN |
| | | LIQQKNPVWL | ISSAVLDQRLG | WLWRNREC | KIYKDLNGY | PVTNEDRVI | FLGSKKRRI |
| | | DLLTEFGLDVK | WAHNSAVYR | RNITIEVTTSE | KELAHRYAK | DSFHRIPISS | KRSMARAE |
| | | SLARLQRAIEE | HTLWLLNPFK | LDTFVVEHA | NILLGTWL | GSSRQDFILF | LFGVEQSLP |
| | | QLPENSFPDS | WQSQPVCILL | QKLSWYGH | WRNRECR | IQKELADERA | VTNEDRVI |
| | | VSTYSKQLRFP | LIQQKNPVWL | WDGDSTECL | NITIEVTTSE | KSGFNSYGF | DSFHRIPISS |
| | | WGDDYVSITP | DLLTEFGLDVK | ERLTAYLERA | LDTFVVEH | ATNQEKRAT | GSSRQDFIL |
| | | VVSHALQCEL | SLARLQRAIEE | LSDPTEYFY | AQKLSWYG | VPDLRFNLFE | FIQKELADE |
| | | EIRARSPENKF | QLPENSFPDS | MDVKAKMR | HWDGDST | EDSF | RAKSGFNS |
| | | SFVSSSLPNSA | VSTYSKQLRFP | VGWGDEVY | ECLERLTAY | (SEQ ID | YGFATNQE |
| | | SIGNLCGSLG | WGDDYVSITP | PSQEFLDSRE | LERALSDPT | NO: 407) | KRATVPDL |
| | | GYMRVLNYPL | VVSHALQCEL | DGIPTKQLAT | EYFYMDVK | | RFNLFEEDS |
| | | GVKQAKGGTL | EIRARSPENKF | VELLSGKETV | AKMRVGW | | F (SEQ ID |
| | | TENRQKSGHY | SFVSSSLPNSA | AFHGQKVG | GDEVYPSQ | | NO: 408) |
| | | FDDYQVTNAK | SIGNLCGSLG | AALQSIDDW | EFLDSREDG | | |
| | | ICQVLNRLIGS | GYMRVLNYPL | WNENADKP | IPTKQLATV | | |
| | | EPSKTQRQRE | GVKQAKGGTL | LRVNEYGAD | ELLSGKETV | | |
| | | RARKVRSKILR | TENRQKSGHY | REYVIARRHV | AFHGQKVG | | |
| | | KQIALWMLPL | FDDYQVTNAK | THGNDFYQL | AALQSIDD | | |
| | | IELRDIAESEP | ICQVLNRLIGS | VRNTENWIE | WWNENAD | | |
| | | NQQQLEHDD | EPSKTQRQRE | TMTASRTIP | KPLRVNEY | | |
| | | TLAQAFLSLPE | RARKVRSKILR | NDVHFIMSV | GADREYVIA | | |
| | | WELGSLAGEF | KQIALWMLPL | LIKGGLFNCA | RRHVTHGN | | |
| | | NRRLHLAFQN | IELRDIAESEP | KAN (SEQ ID | DFYQLVRN | | |
| | | NIYSAKFAYHP | NQQQLEHDD | NO: 405) | TENWIETM | | |
| | | KLMQVAKAQ | TLAQAFLSLPE | | TASRTIPND | | |
| | | VTWVLEQLSK | WELGSLAGEF | | VHFIMSVLI | | |
| | | PINNQDTVTG | NRRLHLAFQN | | KGGLFNCA | | |
| | | EQYIYLSSMR | NIYSAKFAYHP | | KAN (SEQ | | |
| | | VQDAVAMSN | KLMQVAKAQ | | ID NO: 406) | | |
| | | PCLCGVPSLTA | VTWVLEQLSK | | | | |
| | | IWGFMHDYQ | PINNQDTVTG | | | | |
| | | RQFNQLVNN | EQYIYLSSMR | | | | |
| | | DSPVEFSSFAF | VQDAVAMSN | | | | |
| | | YVRNENIQST | PCLCGVPSLTA | | | | |
| | | AKLTEPNSIAK | IWGFMHDYQ | | | | |
| | | ARTVSNAKRP | RQFNQLVNN | | | | |
| | | TIRSKRLADLEI | DSPVEFSSFAF | | | | |
| | | DLVIRVHSESR | YVRNENIQST | | | | |
| | | ISDFRSALKTA | AKLTEPNSIAK | | | | |
| | | LPVAFAGGAL | ARTVSNAKRP | | | | |
| | | YQPQLSTQIE | TIRSKRLADLEI | | | | |
| | | WLRTFTGRSE | DLVIRVHSESR | | | | |
| | | LFHVLKGLPAY | ISDFRSALKTA | | | | |
| | | GRWLYPSEKQ | LPVAFAGGAL | | | | |
| | | PTNFDELERLL | YQPQLSTQIE | | | | |
| | | TQDDDNLLVS | WLRTFTGRSE | | | | |
| | | LGYHLLEHPTK | LFHVLKGLPAY | | | | |
| | | RDNAITGCHA | GRWLYPSEKQ | | | | |
| | | YAENAIGLAK | PTNFDELERLL | | | | |
| | | RINPIEVRFSG | TQDDDNLLVS | | | | |
| | | RDHFLNHAF | LGYHLLEHPTK | | | | |
| | | WSIECSSETILI | RDNAITGCHA | | | | |
| | | KNYRD (SEQ | YAENAIGLAK | | | | |
| | | ID NO: 403) | RINPIEVRFSG | | | | |
| | | | RDHFLNHAF | | | | |
| | | | WSIECSSETILI | | | | |
| | | | KNYRD (SEQ | | | | |
| | | | ID NO: 404) | | | | |
| 28 | V.hyugaensis_ | MTKLSDLLAIE | MPKKKRKVGS | MELCTQLNY | MPKKKRKV | MTKRYYFCIR | MPKKKRKV |
| | 151112A_ | DEAVKQVTLK | GDYKDDDDK | VRSLSAGKA | GSGELCTQL | YTPVQADYE | GSGTKRYYF |
| | GCA_ | KMFMPYTED | DYKDDDDKD | YFYYLSKSGE | NYVRSLSA | LLAGRCISQ | CIRYTPVQA |
| | 000818475.1 | VCVEGCEKEA | YKDDDDKGS | MCPLEIDRT | GKAYFYYLS | MHLFMVNN | DYELLAGRC |
| | | LTILLNLSSSH | GTKLSDLLAIE | RLRAPKGGY | KSGEMCPL | RQSINKIGVS | ISQMHLFM |
| | | QADRCSDWL | DEAVKQVTLK | AEAYKGSKF | EIDRTRLRA | FPDWSDVTV | VNNRQSIN |
| | | DLARAKRHLK | KMFMPYTED | VEKNVAPQD | PKGGYAEA | GQTIAFVAE | KIGVSFPD |
| | | AAENLEASLD | VCVEGCEKEA | LAYSNPQFIE | YKGSKFVEK | DKEMMIGLS | WSDVTVG |
| | | EIKWFHTHNL | LTILLNLSSSH | ECYVKPGVD | NVAPQDLA | FQPYFSLMV | QTIAFVAED |
| | | KFPDCRVKDQ | QADRCSDWL | DIYCAFPLRIR | YSNPQFIEE | NEGLFEISSV | KEMMIGLS |
| | | RIVAQALTTTE | DLARAKRHLK | ANSLTPDTCS | CYVKPGVD | CEVPDNAIE | FQPYFSLM |
| | | VFISSGVLEQR | AAENLEASLD | DDEVRSKLSL | DIYCAFPLRI | VRFTRNQTI | VNEGLFEIS |
| | | LGWAHNSAV | EIKWFHTHNL | LANTYKELN | RANSLTPDT | GKSFLGSKKR | SVCEVPDN |
| | | YRHTLWLLNP | KFPDCRVKDQ | GYQELAHRY | CSDDEVRS | RIKRSMARA | AIEVRFTRN |
| | | FSWQSQPVCI | RIVAQALTTTE | AKNILLGTW | KLSLLANTY | ELSGVEPSLP | QTIGKSFLG |
| | | LSLIKQESSIWI | VFISSGVLEQR | LWRNRECR | KELNGYQE | ATNEERVVD | SKKRRIKRS |
| | | ELLKEFGLSAK | LGWAHNSAV | QLSIEVTTSD | LAHRYAKNI | SFHRIPISSAS | MARAELSG |
| | | SLARLKHTIEE | YRHTLWLLNP | SQTLIEENAT | LLGTWLWR | SGEDYILFLQ | VEPSLPATN |
| | | QLPDNHFPD | FSWQSQPVCI | RLSWYGHW | NRECRQLSI | KELVGERGA | EERVVDSF |
| | | NVSSYSKQLR | LSLIKQESSIWI | DEASAECLEK | EVTTSDSQT | ANFNSYGLA | HRIPISSASS |
| | | FPWGDNYISL | ELLKEFGLSAK | LTAYLMRAL | LIEENATRL | TNQERKGTV | GEDYILFLQ |
| | | TPVVSHAIQS | SLARLKHTIEE | SDPTEYFYM | SWYGHWD | PELRF (SEQ | KELVGERG |
| | | ELEVRSRNRES | QLPDNHFPD | DVKAKIGVG | EASAECLEK | ID NO: 413) | AANFNSYG |
| | | KLSFVSSSLPN | NVSSYSKQLR | WGDEVYPS | LTAYLMRA | | LATNQERK |
| | | SASIGNLCGSL | FPWGDNYISL | QEFLDDQEN | LSDPTEYFY | | GTVPELRF |
| | | GGNMKALNY | TPVVSHAIQS | GAPTKQLAT | MDVKAKIG | | (SEQ ID |
| | | PLDVKPARGG | ELEVRSRNRES | VELLNGKET | VGWGDEV | | NO: 414) |
| | | TLPESRKKSGH | KLSFVSSSLPN | AAFHGQKIG | YPSQEFLDD | | |
| | | YFDDYQVTNT | SASIGNLCGSL | AALQSIDDW | QENGAPTK | | |
| | | KVCQVLNHLI | GGNMKALNY | WHEEADKPL | QLATVELLN | | |
| | | GSEPSKTQKQ | PLDVKPARGG | RVNEYGADR | GKETAAFH | | |
| | | RESARKVRSKI | TLPESRKKSGH | EYVIARRHVS | GQKIGAAL | | |
| | | LRKQIALWML | YFDDYQVTNT | YGNDFYQLV | QSIDDWW | | |
| | | PLIELRDIVDA | KVCQVLNHLI | RNTENWIET | HEEADKPL | | |
| | | DPNQQQLEH | GSEPSKTQKQ | MTASQTIPN | RVNEYGAD | | |
| | | DDTLAQAFLT | RESARKVRSKI | DVHFIMSVLI | REYVIARRH | | |
| | | QPESDLGSLA | LRKQIALWML | KGGLFNCSK | VSYGNDFY | | |
| | | SEFNRHLHLTF | PLIELRDIVDA | AK (SEQ ID | QLVRNTEN | | |
| | | QNNKYAAKF | DPNQQQLEH | NO: 411) | WIETMTAS | | |
| | | AYHPKLMQLV | DDTLAQAFLT | | QTIPNDVH | | |
| | | KAQIVWILEQ | QPESDLGSLA | | FIMSVLIKG | | |
| | | LSKPTGNADK | SEFNRHLHLTF | | GLFNCSKAK | | |
| | | VTGEQYIYLSS | QNNKYAAKF | | (SEQ ID | | |
| | | MKVQDAVA | AYHPKLMQLV | | NO: 412) | | |
| | | MSSPYLCGAP | KAQIVWILEQ | | | | |
| | | SLTAIWGFMH | LSKPTGNADK | | | | |
| | | RYQREFNKLV | VTGEQYIYLSS | | | | |
| | | NCNSLFEFSSF | MKVQDAVA | | | | |
| | | SFYVRSEKIQP | MSSPYLCGAP | | | | |
| | | TAKLTEPNSV | SLTAIWGFMH | | | | |
| | | AKARTVSNAK | RYQREFNKLV | | | | |
| | | RPTIRSERLAD | NCNSLFEFSSF | | | | |
| | | LEIDLVIRVHS | SFYVRSEKIQP | | | | |
| | | DSRISDFKAAL | TAKLTEPNSV | | | | |
| | | KTALPVAFAG | AKARTVSNAK | | | | |
| | | GALYQPQLST | RPTIRSERLAD | | | | |
| | | QVEWLKTFTS | LEIDLVIRVHS | | | | |
| | | RSELFHVIKGL | DSRISDFKAAL | | | | |
| | | PAYGRWLYPS | KTALPVAFAG | | | | |
| | | ESQPSNFDEL | GALYQPQLST | | | | |
| | | ERLITKDADNL | QVEWLKTFTS | | | | |
| | | PVSIGYHLLEC | RSELFHVIKGL | | | | |
| | | PTKRCNSITDC | PAYGRWLYPS | | | | |
| | | HAYAENAIGL | ESQPSNFDEL | | | | |
| | | AKKVNPIEVRF | ERLITKDADNL | | | | |
| | | SGRDHFFNHA | PVSIGYHLLEC | | | | |
| | | FWSIECSSETIL | PTKRCNSITDC | | | | |
| | | IKNYRD (SEQ | HAYAENAIGL | | | | |
| | | ID NO: 409) | AKKVNPIEVRF | | | | |
| | | | SGRDHFFNHA | | | | |
| | | | FWSIECSSETIL | | | | |
| | | | IKNYRD (SEQ | | | | |
| | | | ID NO: 410) | | | | |
| 29 | V. | MTKLSDLLTIE | MPKKKRKVGS | MELCTQLNY | MPKKKRKV | MTTRYYFCIR | MPKKKRKV |
| | crassostreae_ | DEAVKQSALK | GDYKDDDDK | VRSLSAGKA | GSGELCTQL | YTPVQADYE | GSGTTRYYF |
| | J5_20_ | KMFMPYTED | DYKDDDDKD | YFYYLSKSGE | NYVRSLSA | LLAGRCISQ | CIRYTPVQA |
| | GCA_ | VCVEGCEKEA | YKDDDDKGS | MCPLEIDRT | GKAYFYYLS | MHLFMVNN | DYELLAGRC |
| | 001048515.1 | LTILLNLSSSH | GTKLSDLLTIE | RLRAPKGGY | KSGEMCPL | RQAINKIGVS | ISQMHLFM |
| | | QADRCSDWL | DEAVKQSALK | AEAYKGGKF | EIDRTRLRA | FPDWSDVTV | VNNRQAIN |
| | | DVARAKRHLK | KMFMPYTED | VGKNVAPQ | PKGGYAEA | GQTIAFVAE | KIGVSFPD |
| | | AAENLEASLD | VCVEGCEKEA | DLAYSNPQFI | YKGGKFVG | DKEMMVGL | WSDVTVG |
| | | EIKWFHTHNL | LTILLNLSSSH | EECYVKPGV | KNVAPQDL | SFQPYFSVM | QTIAFVAED |
| | | KFPDCRVKDQ | QADRCSDWL | DDIYCAFPLR | AYSNPQFIE | VNEGLFEISS | KEMMVGL |
| | | RIIAQPLVTTE | DVARAKRHLK | IRANSLTPDT | ECYVKPGV | VCEVPDTAV | SFQPYFSV |
| | | AFISNAVLEQR | AAENLEASLD | CSDDEVRSK | DDIYCAFPL | EVRFTRNQTI | MVNEGLFE |
| | | LGWAHNSAV | EIKWFHTHNL | LSLLAKTYEEL | RIRANSLTP | GKSFLGSKKR | ISSVCEVPD |
| | | YRHTLWLLNP | KFPDCRVKDQ | NGYQELALR | DTCSDDEV | RIKRSMARA | TAVEVRFTR |
| | | FRWQSQSVSL | RIIAQPLVTTE | YAKNILLGR | RSKLSLLAK | ELSGVESSLP | NQTIGKSFL |
| | | LSLVQQETSV | AFISNAVLEQR | WLWRNREC | TYEELNGY | VTNEERVIDS | GSKKRRIKR |
| | | WVELLKEFGL | LGWAHNSAV | RKLSIEVTTS | QELALRYAK | FHRIPISSGSS | SMARAELS |
| | | GIKSLARLKHT | YRHTLWLLNP | DSQILIVENA | NILLGRWL | AQDYILFVQ | GVESSLPVT |
| | | IEEQLPENSFP | FRWQSQSVSL | TRLSWYGH | WRNRECRK | KESVGERVA | NEERVIDSF |
| | | DSVSTYSKQL | LSLVQQETSV | WGEASEECL | LSIEVTTSDS | ANFNSYGLA | HRIPISSGSS |
| | | RFPWGDDYV | WVELLKEFGL | EKLTAYLMR | QILIVENAT | TNQESRGTV | AQDYILFVQ |
| | | SVTPVVSHAI | GIKSLARLKHT | ALSDPTEYFY | RLSWYGH | PDLRF (SEQ | KESVGERV |
| | | QRELEVRSRS | IEEQLPENSFP | MDVKAKIGV | WGEASEEC | ID NO: 419) | AANFNSYG |
| | | RESKLSFVSSS | DSVSTYSKQL | GWGDEVYP | LEKLTAYLM | | LATNQESR |
| | | LPNSASIGNLC | RFPWGDDYV | SQEFLGSRE | RALSDPTEY | | GTVPDLRF |
| | | GSLGGHMKV | SVTPVVSHAI | DGVPTKQLA | FYMDVKAK | | (SEQ ID |
| | | LNYPLDVKPA | QRELEVRSRS | TVELLNGKET | IGVGWGDE | | NO: 420) |
| | | QGGTLTESRK | RESKLSFVSSS | VAFHGQKV | VYPSQEFLG | | |
| | | KSGHYFDDYQ | LPNSASIGNLC | GAALQSIDD | SREDGVPT | | |
| | | VTNAKICQVL | GSLGGHMKV | WWHENAD | KQLATVELL | | |
| | | NHLIGSEPSKT | LNYPLDVKPA | KPLRVNEYG | NGKETVAF | | |
| | | QKQRESARKV | QGGTLTESRK | ADREYVIARR | HGQKVGA | | |
| | | RSKILRKQIAL | KSGHYFDDYQ | HVSYGNDFY | ALQSIDDW | | |
| | | WMLPLIELRD | VTNAKICQVL | QLVRNTEN | WHENADK | | |
| | | IVDADPNQQ | NHLIGSEPSKT | WIETMTASQ | PLRVNEYG | | |
| | | QLEHDGSLVQ | QKQRESARKV | TIPNDVHFI | ADREYVIAR | | |
| | | SFLALPESDLG | RSKILRKQIAL | MSVLIKGGLF | RHVSYGND | | |
| | | SLASEFNRRLH | WMLPLIELRD | NCSKAK | FYQLVRNT | | |
| | | LTFQNNKYAA | IVDADPNQQ | (SEQ ID | ENWIETMT | | |
| | | KFAYHPKLMQ | QLEHDGSLVQ | NO: 417) | ASQTIPND | | |
| | | VVKAQIVWIL | SFLALPESDLG | | VHFIMSVLI | | |
| | | EQLSKPNGNE | SLASEFNRRLH | | KGGLFNCS | | |
| | | DKVTGEQYIYL | LTFQNNKYAA | | KAK (SEQ | | |
| | | SSMRVQDAV | KFAYHPKLMQ | | ID NO: 418) | | |
| | | AMSSPYLCGA | VVKAQIVWIL | | | | |
| | | PSLAAIWGFM | EQLSKPNGNE | | | | |
| | | HHYQREFNKL | DKVTGEQYIYL | | | | |
| | | VNCDSPFEFSS | SSMRVQDAV | | | | |
| | | FSFYVRSENIQ | AMSSPYLCGA | | | | |
| | | SIAKLTEPNSV | PSLAAIWGFM | | | | |
| | | AKARTVSNAK | HHYQREFNKL | | | | |
| | | RPTIRSERLAD | VNCDSPFEFSS | | | | |
| | | LEIDLVIRIHSD | FSFYVRSENIQ | | | | |
| | | SRISDFKSALK | SIAKLTEPNSV | | | | |
| | | TALPVAFAGG | AKARTVSNAK | | | | |
| | | ALYQPQLSTQI | RPTIRSERLAD | | | | |
| | | EWLRTFTSRS | LEIDLVIRIHSD | | | | |
| | | ELFHVLKGLPA | SRISDFKSALK | | | | |
| | | YGRWLYPSEN | TALPVAFAGG | | | | |
| | | QSSDFDDLEH | ALYQPQLSTQI | | | | |
| | | LITKDADNLPV | EWLRTFTSRS | | | | |
| | | SIGYHLLERPT | ELFHVLKGLPA | | | | |
| | | KRDNSITSCH | YGRWLYPSEN | | | | |
| | | AYAENVIGLAL | QSSDFDDLEH | | | | |
| | | RVSPIEVRFSG | LITKDADNLPV | | | | |
| | | RDHFLNHAF | SIGYHLLERPT | | | | |
| | | WSIECSSETILI | KRDNSITSCH | | | | |
| | | KNYRD (SEQ | AYAENVIGLAL | | | | |
| | | ID NO: 415) | RVSPIEVRFSG | | | | |
| | | | RDHFLNHAF | | | | |
| | | | WSIECSSETILI | | | | |
| | | | KNYRD (SEQ | | | | |
| | | | ID NO: 416) | | | | |
| 30 | A.salmonicida | MQLREWFNT | MPKKKRKVGS | MSYSRSLSP | MPKKKRKV | MNNERFFFV | MPKKKRKV |
| | strain | SDKAERDKAL | GDYKDDDDK | GKAVFFYTTP | GSGSYSRSL | VRYLPSRADS | GSGNNERF |
| | AJ83 | RRAFVPFTPDI | DYKDDDDKD | ECDFVPLRVE | SPGKAVFFY | ALLAGRCISQ | FFVVRYLPS |
| | | EIAGDEWLAL | YKDDDDKGS | VARVLGQKC | TTPECDFVP | LHGYLLRNS | RADSALLA |
| | | VVLLNLTLKRG | GQLREWFNT | GFSEGFDAH | LRVEVARVL | HVQIGVSFP | GRCISQLHG |
| | | QGDELTDKRH | SDKAERDKAL | FQPKTLERHE | GQKCGFSE | DWSDTQLG | YLLRNSHV |
| | | AKALLLDQKH | RRAFVPFTPDI | LAYGNPQTIE | GFDAHFQP | SYIGFVSAEK | QIGVSFPD |
| | | LEKCVKQVR | EIAGDEWLAL | VCYVPPNVH | KTLERHELA | DHLDHFRQR | WSDTQLGS |
| | | WLHSHNLKYP | VVLLNLTLKRG | EIYCRFSLRV | YGNPQTIEV | AYFQIMQED | YIGFVSAEK |
| | | DSRVSHQRLV | QGDELTDKRH | KANALGPTV | CYVPPNVH | GLFSLTTTLE | DHLDHFRQ |
| | | IASPPQIPGVV | AKALLLDQKH | CSDSEVMQT | EIYCRFSLRV | VPIGCAEVRF | RAYFQIMQ |
| | | TSAGLPMRLG | LEKCVKQVR | LVNLSRCYQ | KANALGPT | VRNQGLAKL | EDGLFSLTT |
| | | WANNSADIN | WLHSHNLKYP | DRGGFIELAR | VCSDSEVM | FAGERRRRL | TLEVPIGCA |
| | | HAKLFCSSFLY | DSRVSHQRLV | RYSRNLIMA | QTLVNLSRC | ARAKRRAEA | EVRFVRNQ |
| | | HGVTTNLALQ | IASPPQIPGVV | TWLWRNRQ | YQDRGGFI | RGDVFLPQS | GLAKLFAGE |
| | | LATDVPAPA | TSAGLPMRLG | SQGTRIEIHT | ELARRYSRN | PPEHRDVLQ | RRRRLARA |
| | | WTTAFRKLGL | WANNSADIN | SQGSRYMID | LIMATWL | FHRVLMQS | KRRAEARG |
| | | ADSAIAALQS | HAKLFCSSFLY | DVRHLDWQ | WRNRQSQ | QSNNQDFV | DVFLPQSPP |
| | | QLAQLLATST | HGVTTNLALQ | GQWPASAQ | GTRIEIHTS | MHIEKEPYD | EHRDVLQF |
| | | VPAEVSPYSK | LATDVPAPA | EQWLQLAD | QGSRYMID | NSDSNTGFN | HRVLMQS |
| | | QVRFWYQGD | WTTAFRKLGL | EMATALTRP | DVRHLDW | NYGLACRVQ | QSNNQDFV |
| | | YCAITPVVSH | ADSAIAALQS | DLFWFADVT | QGQWPAS | HRGSVPELA | MHIEKEPY |
| | | GLMSQLHQLI | QLAQLLATST | AVMKTAFC | AQEQWLQ | SIVATLF | DNSDSNTG |
| | | YEKRIPHLIISH | VPAEVSPYSK | QEIYPSQAFT | LADEMATA | (SEQ ID | FNNYGLAC |
| | | DHPASVGSLV | QVRFWYQGD | ERPDNHTEP | LTRPDLFW | NO: 425) | RVQHRGSV |
| | | GAVGGKIAVL | YCAITPVVSH | SKKLATVECT | FADVTAVM | | PELASIVATL |
| | | HYPPPVSVEK | GLMSQLHQLI | DGQLAACLT | KTAFCQEIY | | F (SEQ ID |
| | | RRNFSQSRAT | YEKRIPHLIISH | AQKLGAALQ | PSQAFTERP | | NO: 426) |
| | | RINQGDSLFD | DHPASVGSLV | KIDDWWGE | DNHTEPSK | | |
| | | RTILRDQIFIH | GAVGGKIAVL | EVDEPLRVH | KLATVECTD | | |
| | | ALEHLIAPSGL | HYPPPVSVEK | EYAADPKHQ | GQLAACLT | | |
| | | TRRQRKQSHL | RRNFSQSRAT | TSMRHPVSG | AQKLGAAL | | |
| | | SALRYLRRQLA | RINQGDSLFD | LDFYHLLSRT | QKIDDWW | | |
| | | CWIAPLIEWR | RTILRDQIFIH | DELVAQMES | GEEVDEPLR | | |
| | | DEVEQNQGA | ALEHLIAPSGL | SPESSDIHRD | VHEYAADP | | |
| | | LPSIDPSRVE | TRRQRKQSHL | IHYLMAVLV | KHQTSMR | | |
| | | WQVLSCPQS | SALRYLRRQLA | KGGLFQKGR | HPVSGLDF | | |
| | | ELPSLGIALAE | CWIAPLIEWR | S (SEQ ID | YHLLSRTDE | | |
| | | SCHLALQSHP | DEVEQNQGA | NO: 423) | LVAQMESS | | |
| | | ATRRLAFHPR | LPSIDPSRVE | | PESSDIHRD | | |
| | | LLMPIKTQLR | WQVLSCPQS | | IHYLMAVL | | |
| | | WLLNKLALDE | ELPSLGIALAE | | VKGGLFQK | | |
| | | SVPPQTATCC | SCHLALQSHP | | GRS (SEQ | | |
| | | YLHLSGLRVY | ATRRLAFHPR | | ID NO: 424) | | |
| | | DAVALANPYL | LLMPIKTQLR | | | | |
| | | CGIPSLSALAG | WLLNKLALDE | | | | |
| | | FCHDYERRLT | SVPPQTATCC | | | | |
| | | AVLKRSVRLT | YLHLSGLRVY | | | | |
| | | GVAWYLRDC | DAVALANPYL | | | | |
| | | HLQPAKNLPE | CGIPSLSALAG | | | | |
| | | PSSPLSAHEVS | FCHDYERRLT | | | | |
| | | AIRRPGLIDSK | AVLKRSVRLT | | | | |
| | | HCDLGMDLV | GVAWYLRDC | | | | |
| | | LALHVDADHP | HLQPAKNLPE | | | | |
| | | AFSADEQNLL | PSSPLSAHEVS | | | | |
| | | QAAFPSRFAG | AIRRPGLIDSK | | | | |
| | | GCLHPPSLYE | HCDLGMDLV | | | | |
| | | GQPWCNIYT | LALHVDADHP | | | | |
| | | NRGALFSTLSR | AFSADEQNLL | | | | |
| | | LPRSGCWVYP | QAAFPSRFAG | | | | |
| | | HLSQVTDLED | GCLHPPSLYE | | | | |
| | | FFETFSTDRRL | GQPWCNIYT | | | | |
| | | RPISAGYVFLE | NRGALFSTLSR | | | | |
| | | PPQLRAGSVE | LPRSGCWVYP | | | | |
| | | KHHAYAESAL | HLSQVTDLED | | | | |
| | | GLALCINPVE | FFETFSTDRRL | | | | |
| | | MRLTGNNHF | RPISAGYVFLE | | | | |
| | | FKHGFWQLN | PPQLRAGSVE | | | | |
| | | VSNGAMLMT | KHHAYAESAL | | | | |
| | | GVGNREPPH | GLALCINPVE | | | | |
| | | RGTM (SEQ | MRLTGNNHF | | | | |
| | | ID NO: 421) | FKHGFWQLN | | | | |
| | | | VSNGAMLMT | | | | |
| | | | GVGNREPPH | | | | |
| | | | RGTM (SEQ | | | | |
| | | | ID NO: 422) | | | | |
| 33 | Klebsiella | MHIRELLKIKD | MPKKKRKVGS | MELCTHLSY | MPKKKRKV | MRMTRYFFS | MPKKKRKV |
| | oxytoca | HSERDRALRH | GDYKDDDDK | MRSISPGKA | GSGELCTHL | VYYLPEDAD | GSGRMTRY |
| | strain 67 | GFSPIREKIDM | DYKDDDDKD | VFYYKRPECE | SYMRSISPG | YPLLAGRCIS | FFSVYYLPE |
| | Ga0227227_119 | EGFEYETLVVL | YKDDDDKGS | FVPLEIQTSKI | KAVFYYKRP | TLHGYTSHH | DADYPLLA |
| | | LNMTLKRDLV | GHIRELLKIKD | RGQKCSYSE | ECEFVPLEI | PDTRIGVSFP | GRCISTLHG |
| | | HNLFDVRLAR | HSERDRALRH | GFRENLQPR | QTSKIRGQK | DWTDTTLGR | YTSHHPDT |
| | | QLLFDKNHLA | GFSPIREKIDM | KLQQHDLAY | CSYSEGFRE | TIAFVSVNRS | RIGVSFPD |
| | | HCVNAVRWL | EGFEYETLVVL | ANPLTIEICY | NLQPRKLQ | HLEQLKERA | WTDTTLGR |
| | | HTHNLKYPDS | LNMTLKRDLV | VPADVNEIY | QHDLAYAN | YFKILKEEKIF | TIAFVSVNR |
| | | RVRGQRLIICS | HNLFDVRLAR | CRFTLRIEAN | PLTIEICYVP | SISPVLKVPE | SHLEQLKER |
| | | PAVIPGIVSSA | QLLFDKNHLA | SLRPYVCGD | ADVNEIYCR | YCPDVMFIR | AYFKILKEEK |
| | | DLPQEMGWA | HCVNAVRWL | PHVLNTLTEL | FTLRIEANSL | NQTIAKCFV | IFSISPVLKV |
| | | NNGADINFAR | HTHNLKYPDS | ALEYKKHDG | RPYVCGDP | KERKRRLERA | PEYCPDVM |
| | | LFCSFFRHNG | RVRGQRLIICS | YKELAKRYST | HVLNTLTEL | KRRAEARGE | FIRNQTIAK |
| | | SITCLAKLLTE | PAVIPGIVSSA | NLLMGSWL | ALEYKKHD | VFQPRVNSP | CFVKERKRR |
| | | GCSGIVKALER | DLPQEMGWA | WRNRFTQST | GYKELAKRY | LRSIEAFHGIF | LERAKRRAE |
| | | LGTSTDDICLL | NNGADINFAR | QLEIKTSLNS | STNLLMGS | MQSISNGCS | ARGEVFQP |
| | | RVAIANNISES | LFCSFFRHNG | TYRILDSREL | WLWRNRF | FLLHIQKKEA | RVNSPLRSI |
| | | VIPSDVSIYSR | SITCLAKLLTE | NWSEAWPE | TQSTQLEIK | RIQSNHMYC | EAFHGIFM |
| | | QLRGFLQGKD | GCSGIVKALER | SEQRQRELLE | TSLNSTYRIL | SYGLASNEV | QSISNGCSF |
| | | VAITPVVSHAL | LGTSTDDICLL | REIETALSEP | DSRELNWS | YTGHVPDLS | LLHIQKKEA |
| | | MARLQQLIYQ | RVAIANNISES | GVFWGADVI | EAWPESEQ | SVVKKLF | RIQSNHMY |
| | | QRKPHIIIRHD | VIPSDVSIYSR | ATLQTSFCQ | RQRELLERE | (SEQ ID | CSYGLASNE |
| | | HPASMGNLV | QLRGFLQGKD | EIYPSQKFIEK | IETALSEPG | NO: 431) | VYTGHVPD |
| | | ASTGGNIAV | VAITPVVSHAL | TVDYSIASRQ | VFWGADVI | | LSSVVKKLF |
| | | MYYPPLVSVH | MARLQQLIYQ | LATTECSNG | ATLQTSFC | | (SEQ ID |
| | | KERSFIHSRVG | QRKPHIIIRHD | KQAACITAQ | QEIYPSQKF | | NO: 432) |
| | | LLQEREHLFD | HPASMGNLV | KIGAALQRID | IEKTVDYSIA | | |
| | | NNVLREKELF | ASTGGNIAV | DWWSADA | SRQLATTEC | | |
| | | NALQNLVSH | MYYPPLVSVH | DYPLRVHEY | SNGKQAAC | | |
| | | NGGSQRQIR | KERSFIHSRVG | GAEPERLTA | ITAQKIGAA | | |
| | | QQRLSALRYL | LLQEREHLFD | RRHPVSGHD | LQRIDDW | | |
| | | RYQLVIWLKP | NNVLREKELF | FYHLLTKADI | WSADADYP | | |
| | | VIECIDALEEN | NALQNLVSH | FLNDFKSKK | LRVHEYGA | | |
| | | REDILSLPESIE | NGGSQRQIR | MKKISGDIHF | EPERLTARR | | |
| | | KKVLTQSVNR | QQRLSALRYL | LMSVLVKGG | HPVSGHDF | | |
| | | LDELSSELAGH | RYQLVIWLKP | LFQKGRGA | YHLLTKADI | | |
| | | FHLSLQHHPL | VIECIDALEEN | (SEQ ID | FLNDFKSKK | | |
| | | FRRFAFHSELV | REDILSLPESIE | NO: 429) | MKKISGDIH | | |
| | | VSVESQLKWI | KKVLTQSVNR | | FLMSVLVK | | |
| | | LKNISRSDPDT | LDELSSELAGH | | GGLFQKGR | | |
| | | PITQSCREFYL | FHLSLQHHPL | | GA (SEQ ID | | |
| | | HLSGLNIYDAS | FRRFAFHSELV | | NO: 430) | | |
| | | AMSNPYLCGI | VSVESQLKWI | | | | |
| | | PSLTALAGFC | LKNISRSDPDT | | | | |
| | | HDYERRVSAL | PITQSCREFYL | | | | |
| | | MEQKVCFTEV | HLSGLNIYDAS | | | | |
| | | AWYIGHYNLI | AMSNPYLCGI | | | | |
| | | SGRQLPAAMI | PSLTALAGFC | | | | |
| | | PERKNTISSLR | HDYERRVSAL | | | | |
| | | RPGITDEKCC | MEQKVCFTEV | | | | |
| | | DMGIELVIKL | AWYIGHYNLI | | | | |
| | | QFPEECKLPES | SGRQLPAAMI | | | | |
| | | GLLYAASPSRF | PERKNTISSLR | | | | |
| | | AGGVLHPPSF | RPGITDEKCC | | | | |
| | | SGEKSWCQLY | DMGIELVIKL | | | | |
| | | SDQDALYSVL | QFPEECKLPES | | | | |
| | | SRLPGSGCWI | GLLYAASPSRF | | | | |
| | | YPVRTTITTLE | AGGVLHPPSF | | | | |
| | | EMFTELSSDY | SGEKSWCQLY | | | | |
| | | RLRPVSSGFIL | SDQDALYSVL | | | | |
| | | LEEMQYRAGS | SRLPGSGCWI | | | | |
| | | LASQHVYAES | YPVRTTITTLE | | | | |
| | | ALGLARCHNP | EMFTELSSDY | | | | |
| | | IEIRLAGKKNF | RLRPVSSGFIL | | | | |
| | | YNQGFWPLD | LEEMQYRAGS | | | | |
| | | YEDRTIIT | LASQHVYAES | | | | |
| | | (SEQ ID | ALGLARCHNP | | | | |
| | | NO: 427) | IEIRLAGKKNF | | | | |
| | | | YNQGFWPLD | | | | |
| | | | YEDRTIIT | | | | |
| | | | (SEQ ID | | | | |
| | | | NO: 428) | | | | |
| 36 | Pseudo. | MIKLECICRHG | MPKKKRKVGS | MELCNILKY | MPKKKRKV | MQRYYFTVH | MPKKKRKV |
| | arctica | EYMHLKELLEI | GDYKDDDDK | DRSLYPSKAV | GSGELCNIL | FLPKQANLA | GSGQRYYF |
| | A 37-1-2 | TDIAERDRLIR | DYKDDDDKD | FFYKTADSDF | KYDRSLYPS | LLTGRCISIM | TVHFLPKQ |
| | chromosome I | RAFNPYTTTID | YKDDDDKGS | VPLEADINKV | KAVFFYKTA | HGFILKHNIE | ANLALLTGR |
| | | ITGCEGNTLIIL | GIKLECICRHG | RGPKSGFTE | DSDFVPLEA | GMGVTFPA | CISIMHGFIL |
| | | LNLTYRKNQV | EYMHLKELLEI | AFTPQFLPK | DINKVRGP | WSDSSIGNV | KHNIEGMG |
| | | DDLLDKQLAK | TDIAERDRLIR | NISPQDLTH | KSGFTEAFT | IAFVHKDME | VTFPAWSD |
| | | QALKSEEHINK | RAFNPYTTTID | NNILTLEECY | PQFLPKNIS | VLNSLKEQA | SSIGNVIAF |
| | | CIKEIAWFHT | ITGCEGNTLIIL | VPPNVEHIFC | PQDLTHNN | YFVDMQDC | VHKDMEVL |
| | | HNLKYPDIRVS | LNLTYRKNQV | RFSLRVQAN | ILTLEECYVP | GFFKISQISTV | NSLKEQAYF |
| | | KQNLAVAPPL | DDLLDKQLAK | SLAPSGCSDP | PNVEHIFCR | PDSCQEVRFI | VDMQDCG |
| | | LDSYVLSSANY | QALKSEEHINK | EVFSLLKELA | FSLRVQAN | RNQSVAKIFT | FFKISQISTV |
| | | PKAYGWSHD | CIKEIAWFHT | TIFKECGGYK | SLAPSGCSD | GESRRRLKRL | PDSCQEVR |
| | | SAKVNFAKLF | HNLKYPDIRVS | ELATRYCRNI | PEVFSLLKEL | QKRALARGE | FIRNQSVAK |
| | | VSYFKWQNQ | KQNLAVAPPL | LLGTWLWR | ATIFKECGG | DFNPKKLEA | IFTGESRRR |
| | | DSCLAQVLAT | LDSYVLSSANY | NQNTGNTQI | YKELATRYC | PREIDIFHRV | LKRLQKRAL |
| | | NSDNWKAAF | PKAYGWSHD | EIKTSKGNRY | RNILLGTWL | AMTSKSSQE | ARGEDFNP |
| | | TSLGLSVKAFK | SAKVNFAKLF | LIDNTRKLA | WRNQNTG | DYILHIQKQD | KKLEAPREI |
| | | SLCVTVKKSLP | VSYFKWQNQ | WESKWASD | NTQIEIKTS | ADCQAEPVL | DIFHRVAM |
| | | EEAIPDSVDRY | DSCLAQVLAT | DQRVLEELS | KGNRYLIDN | SNYGFSSNE | TSKSSQEDY |
| | | SRQIRMPYHD | NSDNWKAAF | NEIESALTDP | TRKLAWES | KFKGTVPDLS | ILHIQKQDA |
| | | GYLAVTPVISH | TSLGLSVKAFK | NVFWSADIT | KWASDDQ | PLIESN (SEQ | DCQAEPVL |
| | | VVQSKIQQAA | SLCVTVKKSLP | AKIEASFCQE | RVLEELSNE | ID NO: 437) | SNYGFSSNE |
| | | IDKRARFSNV | EEAIPDSVDRY | VYPSQILNDK | IESALTDPN | | KFKGTVPDL |
| | | EFTRPAAVSLL | SRQIRMPYHD | VKQGEASKQ | VFWSADIT | | SPLIESN |
| | | AASLGGVVNV | GYLAVTPVISH | FVKSKCADG | AKIEASFCQ | | (SEQ ID |
| | | LNYPPKILNKY | VVQSKIQQAA | RYAVSFNSV | EVYPSQILN | | NO: 438) |
| | | HGLSSSRQFKL | IDKRARFSNV | KIGAALQSID | DKVKQGEA | | |
| | | NNGQTVFNV | EFTRPAAVSLL | DWWDEDAS | SKQFVKSKC | | |
| | | GALLKPEFIKA | AASLGGVVNV | KRLRVHEFG | ADGRYAVS | | |
| | | LEGIIFSNNAL | LNYPPKILNKY | ADKEIGIARR | FNSVKIGAA | | |
| | | ALKQRRQQK | HGLSSSRQFKL | PPDSEQNFY | LQSIDDW | | |
| | | VKNIRDVRSTL | NNGQTVFNV | SIFKNTEWYL | WDEDASKR | | |
| | | LEWFSPIYEW | GALLKPEFIKA | SALKNCITNK | LRVHEFGA | | |
| | | RLDIIETEVGLE | LEGIIFSNNAL | NENIDPAIYY | DKEIGIARR | | |
| | | QLEGTSDQLE | ALKQRRQQK | LFSVLIKGGM | PPDSEQNF | | |
| | | YKILSLSDDEL | VKNIRDVRSTL | FQKKAEAKK | YSIFKNTEW | | |
| | | PLLTIPLFRLLN | LEWFSPIYEW | A (SEQ ID | YLSALKNCI | | |
| | | EMLSDVSMT | RLDIIETEVGLE | NO: 435) | TNKNENID | | |
| | | QRYAFHPQL | QLEGTSDQLE | | PAIYYLFSVL | | |
| | | MSPLKAALQ | YKILSLSDDEL | | IKGGMFQK | | |
| | | WLLINLTDQK | PLLTIPLERLLN | | KAEAKKA | | |
| | | NELIEEDDEHY | EMLSDVSMT | | (SEQ ID | | |
| | | RYLHLSGIRVF | QRYAFHPQL | | NO: 436) | | |
| | | DAQALSNPYC | MSPLKAALQ | | | | |
| | | SGIPSLTAVW | WLLINLTDQK | | | | |
| | | GMLHSYQRKL | NELIEEDDEHY | | | | |
| | | NEALGINVRF | RYLHLSGIRVF | | | | |
| | | TSFSWFIRDYS | DAQALSNPYC | | | | |
| | | AVAGKKLPEL | SGIPSLTAVW | | | | |
| | | SLQGAQQNK | GMLHSYQRKL | | | | |
| | | LKRPGIIDGKY | NEALGINVRF | | | | |
| | | CDLIFDLIIHID | TSFSWFIRDYS | | | | |
| | | GYEDDLQTVD | AVAGKKLPEL | | | | |
| | | SEPDILKAYFP | SLQGAQQNK | | | | |
| | | STFAGGVMH | LKRPGIIDGKY | | | | |
| | | QPQLSSNVN | CDLIFDLIIHID | | | | |
| | | WCYLYSNEN | GYEDDLQTVD | | | | |
| | | QLFEKLKRLPL | SEPDILKAYFP | | | | |
| | | SGCWVMPN | STFAGGVMH | | | | |
| | | DHKIEDLDELL | QPQLSSNVN | | | | |
| | | LLLNNDSKLSP | WCYLYSNEN | | | | |
| | | SMMGYMLLT | QLFEKLKRLPL | | | | |
| | | EPMARVGALE | SGCWVMPN | | | | |
| | | RLHCYAEPAIG | DHKIEDLDELL | | | | |
| | | VVKYETAISVR | LLLNNDSKLSP | | | | |
| | | LKGIGNYFNS | SMMGYMLLT | | | | |
| | | AFWVLDAQE | EPMARVGALE | | | | |
| | | KFMLMKKV | RLHCYAEPAIG | | | | |
| | | (SEQ ID | VVKYETAISVR | | | | |
| | | NO: 433) | LKGIGNYFNS | | | | |
| | | | AFWVLDAQE | | | | |
| | | | KFMLMKKV | | | | |
| | | | (SEQ ID | | | | |
| | | | NO: 434) | | | | |
| 37 | Pseud. | MNLQDALAIE | MPKKKRKVGS | MQLPRHLSY | MPKKKRKV | MKRYYFTITY | MPKKKRKV |
| | translucida | PLKEKTTALRK | GDYKDDDDK | TRSLSPSKAV | GSGQLPRH | LPQSCDVSLL | GSGKRYYFT |
| | KMM 520 | LFVPYTSHVEV | DYKDDDDKD | FFYKTPESDF | LSYTRSLSPS | AGRCIGILHG | ITYLPQSCD |
| | | DGFEELALTVL | YKDDDDKGS | EPLQIEQNKL | KAVFFYKTP | FMSSREISNI | VSLLAGRCI |
| | | INLVYKRSEID | GNLQDALAIE | VGQKSGFGD | ESDFEPLQI | GVCFPKWN | GILHGFMS |
| | | DLTSARTAKS | PLKEKTTALRK | AYQKQNVA | EQNKLVGQ | EQTIGNELAF | SREISNIGV |
| | | VLRDEVLLSKC | LFVPYTSHVEV | KNLAPQDLA | KSGFGDAY | VSTNKKQLT | CFPKWNEQ |
| | | INEVKWFHTH | DGFEELALTVL | FGNPQTIDV | QKQNVAK | NLSQQSYFE | TIGNELAFV |
| | | NLKYPDIRVSH | INLVYKRSEID | CYVPPTVNE | NLAPQDLA | MMAHDKLF | STNKKQLT |
| | | QRLISEVVSED | DLTSARTAKS | LFCRFSLRVE | FGNPQTID | GLSKILEVPV | NLSQQSYFE |
| | | IAGICSRSLPLS | VLRDEVLLSKC | ANCIEPHVC | VCYVPPTV | NQSEVMFV | MMAHDKL |
| | | FGWSHNSAEI | INEVKWFHTH | DDPKVIYWL | NELFCRFSL | RNQSVAKAF | FGLSKILEVP |
| | | NHAKLFLTSF | NLKYPDIRVSH | KRFFETYKKH | RVEANCIEP | VGEKQRRLK | VNQSEVMF |
| | | NWQGEVTCL | QRLISEVVSED | NGLNEVATR | HVCDDPKV | RAKKRAEAR | VRNQSVAK |
| | | ARLLINEEPV | IAGICSRSLPLS | YAKNILMGN | IYWLKRFFE | GEVYNPEYK | AFVGEKQR |
| | | WINLIRAYGFT | FGWSHNSAEI | WLWRNRQS | TYKKHNGL | FEAKDIGHF | RLKRAKKRA |
| | | KKAVLEISGKI | NHAKLFLTSF | PNVDIEILTE | NEVATRYA | HSIPVSSKGN | EARGEVYN |
| | | KQQLPVAEFP | NWQGEVTCL | HAAPIVVEG | KNILMGN | GQSYVLHIQ | PEYKFEAKD |
| | | LEVSSFSPQLQ | ARLLINEEPV | AQKLKWQG | WLWRNRQ | KNENAESIK | IGHFHSIPV |
| | | MPFQQSYLV | WINLIRAYGFT | NWQNNQT | SPNVDIEILT | NQFNNYGF | SSKGNGQS |
| | | VTPVVSHAML | KKAVLEISGKI | ALLTLSESIQE | EHAAPIVVE | ATNQIFLGTV | YVLHIQKNE |
| | | AKIQQLTTDR | KQQLPVAEFP | GLSNPQNYC | GAQKLKW | PSLNTLL | NAESIKNQF |
| | | KLNFALVEHS | LEVSSFSPQLQ | YLDITAKIKN | QGNWQN | (SEQ ID | NNYGFATN |
| | | RPANVGDLAS | MPFQQSYLV | AFSQEVHPS | NQTALLTLS | NO: 443) | QIFLGTVPS |
| | | SVGGNIRVLR | VTPVVSHAML | QKFVDNVEQ | ESIQEGLSN | | LNTLL (SEQ |
| | | YFPKTYSKAV | AKIQQLTTDR | GMSSKQLAY | PQNYCYLDI | | ID NO: 444) |
| | | NRSKVANNDI | KLNFALVEHS | TQVGDKKAA | TAKIKNAFS | | |
| | | EKAFKIRALLS | RPANVGDLAS | SLNSQKVGA | QEVHPSQK | | |
| | | SQFQQALLVL | SVGGNIRVLR | AIQTIDDWY | FVDNVEQG | | |
| | | VGIKQFNTLR | YFPKTYSKAV | EEGYKPLRTH | MSSKQLAY | | |
| | | QKRLARVAAI | NRSKVANNDI | EYGADKQIL | TQVGDKKA | | |
| | | RQVRVSLQL | EKAFKIRALLS | VAHRTPKSH | ASLNSQKV | | |
| | | WLDNILEAKN | SQFQQALLVL | SDFYSLLPRIA | GAAIQTIDD | | |
| | | NAQNQVYPE | VGIKQFNTLR | LHIKHMEKH | WYEEGYKP | | |
| | | WVRHYLDQSI | QKRLARVAAI | GLEQSEQSN | LRTHEYGA | | |
| | | TNCISQFSNVL | RQVRVSLQL | SIHFIAAVLIK | DKQILVAH | | |
| | | NESLGNLSKLK | WLDNILEAKN | GGLFQRSKG | RTPKSHSDF | | |
| | | RFAYHPNLM | NAQNQVYPE | (SEQ ID | YSLLPRIALH | | |
| | | GLFKAQLNYV | WVRHYLDQSI | NO: 441) | IKHMEKHG | | |
| | | FTHCAAEQEIL | TNCISQFSNVL | | LEQSEQSNS | | |
| | | NDEQIVYVHC | NESLGNLSKLK | | IHFIAAVLIK | | |
| | | QDMRVFDAE | RFAYHPNLM | | GGLFQRSK | | |
| | | AMANPYIQG | GLFKAQLNYV | | G (SEQ ID | | |
| | | MPSLTALNGL | FTHCAAEQEIL | | NO: 442) | | |
| | | AHNFERKLKN | NDEQIVYVHC | | | | |
| | | FIDPSIKCIGSA | QDMRVFDAE | | | | |
| | | IYIENYQLHTG | AMANPYIQG | | | | |
| | | KPLPEPSKLKQ | MPSLTALNGL | | | | |
| | | VAGRSHVIRS | AHNFERKLKN | | | | |
| | | GIIDKPKCDITL | FIDPSIKCIGSA | | | | |
| | | DLVFRLFVPN | IYIENYQLHTG | | | | |
| | | TELLDKLNSQL | KPLPEPSKLKQ | | | | |
| | | IKPALPSSFAG | VAGRSHVIRS | | | | |
| | | GTMHPPSLYQ | GIIDKPKCDITL | | | | |
| | | NIDWCHVHT | DLVFRLFVPN | | | | |
| | | KPSELFKKLKA | TELLDKLNSQL | | | | |
| | | KSSNGSWLYP | IKPALPSSFAG | | | | |
| | | SKKVVKSFEQL | GTMHPPSLYQ | | | | |
| | | IDALNSNFNL | NIDWCHVHT | | | | |
| | | RPAAIGLAALE | KPSELFKKLKA | | | | |
| | | EPVKRDAALH | KSSNGSWLYP | | | | |
| | | EYHCYAEPVIG | SKKVVKSFEQL | | | | |
| | | LLECVSNTSVK | IDALNSNFNL | | | | |
| | | YAGAKQFFHD | RPAAIGLAALE | | | | |
| | | AFWVMDVQ | EPVKRDAALH | | | | |
| | | KESMLMKKSK | EYHCYAEPVIG | | | | |
| | | FEYE (SEQ ID | LLECVSNTSVK | | | | |
| | | NO: 439) | YAGAKQFFHD | | | | |
| | | | AFWVMDVQ | | | | |
| | | | KESMLMKKSK | | | | |
| | | | FEYE (SEQ ID | | | | |
| | | | NO: 440) | | | | |
| 38 | Shewanella_ | MVDKLKFQEL | MPKKKRKVGS | MELCNVLKY | MPKKKRKV | MQRYYFMV | MPKKKRKV |
| | piezotolerans_ | LDIDDISERNI | GDYKDDDDK | DRSLYPSKAV | GSGELCNV | RFLPEQANL | GSGQRYYF |
| | WP3_ | VLRRAFTAYT | DYKDDDDKD | FFYKTAESNF | LKYDRSLYP | ALLTGRCISV | MVRFLPEQ |
| | uid58745 | VPLDVTGNEA | YKDDDDKGS | VPLEAEINRI | SKAVFFYKT | MHGFICKHE | ANLALLTGR |
| | | AALTILLNLTY | GVDKLKFQEL | RGQKAGFTE | AESNFVPLE | IQGLGVSFPA | CISVMHGFI |
| | | PRKRVDDLLD | LDIDDISERNI | AFTPQFKSK | AEINRIRGQ | WSDVSIGN | CKHEIQGL |
| | | MRLAKQTLNT | VLRRAFTAYT | NLAPQDLAH | KAGFTEAFT | MIAFVHTDI | GVSFPAWS |
| | | DAHVDACIGE | VPLDVTGNEA | CNPLILEECY | PQFKSKNL | AVLNELRLQ | DVSIGNMI |
| | | VQWLHTHNL | AALTILLNLTY | VPPNVEHIYC | APQDLAHC | GYFQDMQE | AFVHTDIAV |
| | | KYPDIRVSKQ | PRKRVDDLLD | RFSLRVQAN | NPLILEECY | YGAFNIGDV | LNELRLQGY |
| | | RLIAASPLLHP | MRLAKQTLNT | SLKPAGCSEP | VPPNVEHIY | EAVPDSCTE | FQDMQEY |
| | | HVLSSANCIN | DAHVDACIGE | TVFALLEEFA | CRFSLRVQ | VRFKRNQAI | GAFNIGDV |
| | | TLGWSHDSA | VQWLHTHNL | ATFKACGGY | ANSLKPAG | AKMFVGETR | EAVPDSCTE |
| | | KVNLAKLFSC | KYPDIRVSKQ | KELATRYCK | CSEPTVFAL | RRLKRLEKRA | VRFKRNQA |
| | | HFIWQERVCC | RLIAASPLLHP | NVLLGTWL | LEEFAATFK | LARGEVFNP | IAKMFVGE |
| | | LATLLADAPK | HVLSSANCIN | WRNQNTGN | ACGGYKEL | SKSYEPRELD | TRRRLKRLE |
| | | GWKEAFQAL | TLGWSHDSA | SQIEIKTSSG | ATRYCKNV | SFHCIAVGST | KRALARGE |
| | | GMLVKDFMN | KVNLAKLFSC | NCYQIANTR | LLGTWLWR | STEQDFLLHV | VFNPSKSYE |
| | | LCGRIKASLPN | HFIWQERVCC | QLAWDSSW | NQNTGNS | QKENVQKRE | PRELDSFHC |
| | | DDTPNHVDK | LATLLADAPK | PADAQQVLE | QIEIKTSSG | GAEFSQLGL | IAVGSTSTE |
| | | YSIQVRLPYQ | GWKEAFQAL | ELSHEVHQA | NCYQIANT | ATNQLLRGT | QDFLLHVQ |
| | | DGYLAITPVVS | GMLVKDFMN | LTDPAVFWH | RQLAWDSS | VPEFDMF | KENVQKRE |
| | | HALQAEIQQA | LCGRIKASLPN | AKITAKIETAF | WPADAQQ | (SEQ ID | GAEFSQLGL |
| | | AMAKQGRYT | DDTPNHVDK | CQEIYPSQSF | VLEELSHEV | NO: 449) | ATNQLLRG |
| | | NIEFTRPAGVS | YSIQVRLPYQ | GEKAAQGEA | HQALTDPA | | TVPEFDMF |
| | | ELSASLGGNV | DGYLAITPVVS | SKQFAKVKC | VFWHAKIT | | (SEQ ID |
| | | KALNYPPRIEN | HALQAEIQQA | VDGRYAVSF | AKIETAFCQ | | NO: 450) |
| | | AEHGLSDSW | AMAKQGRYT | NSVKIGAAL | EIYPSQSFG | | |
| | | ALKVQSGQTV | NIEFTRPAGVS | QLIDDWWD | EKAAQGEA | | |
| | | LNQGALSQPR | ELSASLGGNV | VDGSKRLRIH | SKQFAKVK | | |
| | | FKRALEGLLSK | KALNYPPRIEN | EYGADKEIG | CVDGRYAV | | |
| | | NFELALKQRR | AEHGLSDSW | VARRAPESK | SFNSVKIGA | | |
| | | QQKVACMRQ | ALKVQSGQTV | QSFYSLFVNA | ALQLIDDW | | |
| | | IRATLTEWLSP | LNQGALSQPR | ELYLAELKQQ | WDVDGSK | | |
| | | LLEWRLEVEE | FKRALEGLLSK | LAEGEYSISP | RLRIHEYGA | | |
| | | NKVNTSELGCI | NFELALKQRR | NIYYLFAVLIK | DKEIGVARR | | |
| | | HGSFEYQFLT | QQKVACMRQ | GGMFQKKA | APESKQSFY | | |
| | | TQKENFVELLS | IRATLTEWLSP | EAKSKSKAEP | SLFVNAELY | | |
| | | PMFSLLNTVL | LLEWRLEVEE | TTAKTTTSKA | LAELKQQL | | |
| | | SNSNTLQKYA | NKVNTSELGCI | TPVKA (SEQ | AEGEYSISP | | |
| | | FHQHLMKPLK | HGSFEYQFLT | ID NO: 447) | NIYYLFAVLI | | |
| | | NSLKWLLDNL | TQKENFVELLS | | KGGMFQK | | |
| | | SKESNAVAIDS | PMFSLLNTVL | | KAEAKSKSK | | |
| | | DEDNQQRYLY | SNSNTLQKYA | | AEPTTAKTT | | |
| | | LKGIRVFDAQ | FHQHLMKPLK | | TSKATPVKA | | |
| | | ALSNPYCAGIP | NSLKWLLDNL | | (SEQ ID | | |
| | | SLTAVWGM | SKESNAVAIDS | | NO: 448) | | |
| | | MHNYQRRLN | DEDNQQRYLY | | | | |
| | | ERLGTQLRLTS | LKGIRVFDAQ | | | | |
| | | FSWFIRQYSSL | ALSNPYCAGIP | | | | |
| | | AGKKLPEYGM | SLTAVWGM | | | | |
| | | QGQKENQFR | MHNYQRRLN | | | | |
| | | RAGIVDNKHS | ERLGTQLRLTS | | | | |
| | | DLVFDLVVHI | FSWFIRQYSSL | | | | |
| | | DGYEEDLDAI | AGKKLPEYGM | | | | |
| | | DNSIDAIKASF | QGQKENQFR | | | | |
| | | PATFAGGVM | RAGIVDNKHS | | | | |
| | | HPPEIGSVDE | DLVFDLVVHI | | | | |
| | | WCELYCSEAS | DGYEEDLDAI | | | | |
| | | LYSKLRRLPAS | DNSIDAIKASF | | | | |
| | | GKWIMPTRY | PATFAGGVM | | | | |
| | | QMDSLDGLL | HPPEIGSVDE | | | | |
| | | QLLKLNVALC | WCELYCSEAS | | | | |
| | | PVMSGYLML | LYSKLRRLPAS | | | | |
| | | GSAESRNYSLE | GKWIMPTRY | | | | |
| | | PLHCYAEPAIG | QMDSLDGLL | | | | |
| | | VVECATAIDIR | QLLKLNVALC | | | | |
| | | LQGMSNFFR | PVMSGYLML | | | | |
| | | RAFWMLDIKE | GSAESRNYSLE | | | | |
| | | TSMLMKRI | PLHCYAEPAIG | | | | |
| | | (SEQ ID | VVECATAIDIR | | | | |
| | | NO: 445) | LQGMSNFFR | | | | |
| | | | RAFWMLDIKE | | | | |
| | | | TSMLMKRI | | | | |
| | | | (SEQ ID | | | | |
| | | | NO: 446) | | | | |
| 40 | V.azureus | MTKLSDLLAIE | MPKKKRKVGS | MRLCNQLN | MPKKKRKV | MTKRYYFSV | MPKKKRKV |
| | strain LC2- | DEVLKQATLK | GDYKDDDDK | YLRSLSTGKA | GSGRLCNQ | KYLPAGADH | GSGTKRYYF |
| | 005 | KMFMPYTED | DYKDDDDKD | YFYSLSSDGTI | LNYLRSLST | DLLAGRCIHE | SVKYLPAGA |
| | | VCVEGFEKEA | YKDDDDKGS | NPIGLDRTRL | GKAYFYSLS | MHLFMINN | DHDLLAGR |
| | | LTILLNLSSNH | GTKLSDLLAIE | RAPKGGYSE | SDGTINPIG | PQAMNKIG | CIHEMHLF |
| | | QADKCADWL | DEVLKQATLK | AYQGNNFSP | LDRTRLRAP | VTFPDWGFT | MINNPQA |
| | | DDARAKNYLN | KMFMPYTED | KNVAPQDLA | KGGYSEAY | SVGQRIAFV | MNKIGVTF |
| | | DSKNLKSSLDE | VCVEGFEKEA | YANPQFIEEC | QGNNFSPK | AESKEMLTA | PDWGFTSV |
| | | IQWFHTHNLK | LTILLNLSSNH | YVRPGVDEIY | NVAPQDLA | LSFQNYFSL | GQRIAFVA |
| | | FPDCRVKDSRI | QADKCADWL | CAFSLRISAN | YANPQFIEE | MVSDGLFEL | ESKEMLTAL |
| | | IAKPLITSESFIS | DDARAKNYLN | SLTPQICNDD | CYVRPGVD | SGVLEVPKT | SFQNYFSL |
| | | SAALEESWG | DSKNLKSSLDE | DVRTQLSQL | EIYCAFSLRI | VRELRFVRN | MVSDGLFE |
| | | WSHNSAVYR | IQWFHTHNLK | ARVYKELGG | SANSLTPQI | QSIGKSFRGS | LSGVLEVPK |
| | | FTLWLLTPFR | FPDCRVKDSRI | YSELANRYAK | CNDDDVRT | KLRRMKRSI | TVRELRFVR |
| | | WQSQSVNLLS | IAKPLITSESFIS | NILLGTWLW | QLSQLARV | ARASALGHA | NQSIGKSFR |
| | | MIKSSNHTW | SAALEESWG | RNRGPRNIKI | YKELGGYSE | LKIPQAREER | GSKLRRMK |
| | | MVLLQDFGL | WSHNSAVYR | EVRTSDSDLF | LANRYAKNI | SIEHFHRVPI | RSIARASAL |
| | | GVEQLADIKEL | FTLWLLTPFR | VIDNALRLS | LLGTWLWR | SSGSSGQTYF | GHALKIPQ |
| | | SYIEMPEESFP | WQSQSVNLLS | WYGQWDN | NRGPRNIKI | LFTQKQVVN | AREERSIEH |
| | | NRVSEYSKQIR | MIKSSNHTW | KSSECLKKLT | EVRTSDSDL | ERSEANFSSY | FHRVPISSG |
| | | LPRKGHYLTIT | MVLLQDFGL | DYFARALSEP | FVIDNALRL | GLATAQERR | SSGQTYFLF |
| | | PVVSHSIQREL | GVEQLADIKEL | TEYFYLDVKA | SWYGQWD | GTVPDLDL | TQKQVVNE |
| | | EIRSRNKESQL | SYIEMPEESFP | EITVGWGDE | NKSSECLKK | (SEQ ID | RSEANFSSY |
| | | RFISSYLPNPA | NRVSEYSKQIR | IYPSQKFLDT | LTDYFARAL | NO: 455) | GLATAQER |
| | | SIGGLCGSLG | LPRKGHYLTIT | KEHDMPTK | SEPTEYFYL | | RGTVPDLD |
| | | GYIKILDYSLGI | PVVSHSIQREL | QFATIELESG | DVKAEITVG | | L (SEQ ID |
| | | KADSKQTLIRY | EIRSRNKESQL | QQTVALHG | WGDEIYPS | | NO: 456) |
| | | HQKRSRFFDD | RFISSYLPNPA | QKVGAALQL | QKFLDTKE | | |
| | | YQLTNNKICQ | SIGGLCGSLG | IDDWWHEE | HDMPTKQF | | |
| | | TLNRLIGFEPL | GYIKILDYSLGI | ADKPLRVNE | ATIELESGQ | | |
| | | KTHKQRNASR | KADSKQTLIRY | YGADREYVI | QTVALHGQ | | |
| | | RIQTKLLRKQI | HQKRSRFFDD | ARRHPKFKN | KVGAALQLI | | |
| | | ALWMLPLIEL | YQLTNNKICQ | DFYHLIQNTE | DDWWHEE | | |
| | | RDLQDAEPN | TLNRLIGFEPL | AWVEDMVV | ADKPLRVN | | |
| | | QQKMEYQDS | KTHKQRNASR | SQTIPNEVHF | EYGADREY | | |
| | | LAQAFLAKPEL | RIQTKLLRKQI | IMSILVKGGL | VIARRHPKF | | |
| | | EFTSLVNDFN | ALWMLPLIEL | FNGSSPKKD | KNDFYHLIQ | | |
| | | QRLHLAFQEN | RDLQDAEPN | K (SEQ ID | NTEAWVED | | |
| | | KFTTQFAYHP | QQKMEYQDS | NO: 453) | MVVSQTIP | | |
| | | KLMQAAKAQI | LAQAFLAKPEL | | NEVHFIMSI | | |
| | | KWVLTQLSKT | EFTSLVNDFN | | LVKGGLFN | | |
| | | EQQEDTSHTE | QRLHLAFQEN | | GSSPKKDK | | |
| | | QYIYLSSLRVQ | KFTTQFAYHP | | (SEQ ID | | |
| | | DVVAMSCPYL | KLMQAAKAQI | | NO: 454) | | |
| | | SGFPSLTAIW | KWVLTQLSKT | | | | |
| | | GFVHQYQREF | EQQEDTSHTE | | | | |
| | | NKRIDSENHV | QYIYLSSLRVQ | | | | |
| | | EFSGFSLFVRS | DVVAMSCPYL | | | | |
| | | EYIQSSAKLSE | SGFPSLTAIW | | | | |
| | | PNSVATKRTIS | GFVHQYQREF | | | | |
| | | NVKRPTTLGQ | NKRIDSENHV | | | | |
| | | RQSDLEMDLV | EFSGFSLFVRS | | | | |
| | | IRVDSKNRLSD | EYIQSSAKLSE | | | | |
| | | YLSELKATFPL | PNSVATKRTIS | | | | |
| | | VFAGGAVYQ | NVKRPTTLGQ | | | | |
| | | PLMSLQIEWL | RQSDLEMDLV | | | | |
| | | KVFSSKSSFFN | IRVDSKNRLSD | | | | |
| | | RIKGLPANGR | YLSELKATFPL | | | | |
| | | WVLPSDEQP | VFAGGAVYQ | | | | |
| | | NCFDDLEQLL | PLMSLQIEWL | | | | |
| | | NQDMDNMP | KVFSSKSSFFN | | | | |
| | | ISIGFHLLEPPK | RIKGLPANGR | | | | |
| | | ARENALTEFH | WVLPSDEQP | | | | |
| | | AYAENALGIA | NCFDDLEQLL | | | | |
| | | KRLSPIDVRFA | NQDMDNMP | | | | |
| | | GRDHFFNHAF | ISIGFHLLEPPK | | | | |
| | | WSLELTDETIL | ARENALTEFH | | | | |
| | | IKNLRD (SEQ | AYAENALGIA | | | | |
| | | ID NO: 451) | KRLSPIDVRFA | | | | |
| | | | GRDHFFNHAF | | | | |
| | | | WSLELTDETIL | | | | |
| | | | IKNLRD (SEQ | | | | |
| | | | ID NO: 452) | | | | |
| 41 | V.fluvialis | MTTLQQLIEID | MPKKKRKVGS | MELCSQLNY | MPKKKRKV | MEPRYYFSIR | MPKKKRKV |
| | strain | DDKLRFSELKK | GDYKDDDDK | VRSLSPGKAY | GSGELCSQL | FIPEHTDNEL | GSGEPRYYF |
| | FDAARGOS_ | AFMPYTRPIEI | DYKDDDDKD | FYYLDDNQR | NYVRSLSPG | LAGRCVSN | SIRFIPEHTD |
| | 104 | DGNEKQALTI | YKDDDDKGS | MCPLQIDRT | KAYFYYLDD | MHGFLSHER | NELLAGRC |
| | | LLNLSLGKPVA | GTTLQQLIEID | HLRAPKSGY | NQRMCPL | NRAFKNSLG | VSNMHGFL |
| | | KDSLDISRAER | DDKLRFSELKK | AEAYTGNFK | QIDRTHLRA | VCFPRWSDK | SHERNRAF |
| | | YFADPENLAK | AFMPYTRPIEI | AKNVAPQDL | PKSGYAEAY | TVGNEIAFVS | KNSLGVCFP |
| | | AEQEIQWFHT | DGNEKQALTI | AFSNPQYIEE | TGNFKAKN | PHESILTGLS | RWSDKTVG |
| | | HNLKFPDCRV | LLNLSLGKPVA | CYVPPGVDD | VAPQDLAF | YQPYFSTMV | NEIAFVSPH |
| | | AEQRILATPLP | KDSLDISRAER | IYCAFSLRIRA | SNPQYIEEC | NEGLFDISDI | ESILTGLSY |
| | | SETPTLTSQSL | YFADPENLAK | NSLFPEVCA | YVPPGVDDI | KIVPDDVEEV | QPYFSTMV |
| | | EQAYGWAHN | AEQEIQWFHT | DAATRETLT | YCAFSLRIR | RFVFNKRIQK | NEGLFDISD |
| | | SAVYKHTVWS | HNLKFPDCRV | GLAETYKELD | ANSLFPEVC | IFNGSKKRRI | IKIVPDDVE |
| | | LNTFLWRGKT | AEQRILATPLP | GYKELAKRY | ADAATRET | KRSMQRAE | EVRFVFNK |
| | | ENVLSLIRLGD | SETPTLTSQSL | AKNILIATWV | LTGLAETYK | MQGRIYTPIS | RIQKIFNGS |
| | | EFWQALLAEF | EQAYGWAHN | WRNRECRNI | ELDGYKELA | TEEREFELFH | KKRRIKRSM |
| | | GFTPTGQFQF | SAVYKHTVWS | EIEVKTEKKN | KRYAKNILI | EIPISSQSSG | QRAEMQG |
| | | KTLVERQLPG | LNTFLWRGKT | WKIADARHL | ATWVWRN | HAFVLHIQR | RIYTPISTEE |
| | | THFPEEVSRYS | ENVLSLIRLGD | EWYGTWDR | RECRNIEIE | QFPVYPEIG | REFELFHEIP |
| | | KQVRFPWRN | EFWQALLAEF | KSQSALDGL | VKTEKKNW | NSFNGYGFA | ISSQSSGHA |
| | | DYLSVTPVVS | GFTPTGQFQF | TDYLEKALSD | KIADARHLE | ANQRWRGT | FVLHIQRQF |
| | | HAMQQELAV | KTLVERQLPG | RSDYFNMDI | WYGTWDR | VPLVTF (SEQ | PVYPEIGNS |
| | | LSRHRECSLRF | THFPEEVSRYS | KAKLTVGW | KSQSALDG | ID NO: 461) | FNGYGFAA |
| | | KSMNYPNSAS | KQVRFPWRN | GDEVYPSQE | LTDYLEKAL | | NQRWRGT |
| | | IGNLCGSLAG | DYLSVTPVVS | FLDVKESGKP | SDRSDYFN | | VPLVTF |
| | | HINVLNYPVD | HAMQQELAV | TKQLAKVVL | MDIKAKLT | | (SEQ ID |
| | | VVPDSYQTLA | LSRHRECSLRF | NGEEESAAY | VGWGDEV | | NO: 462) |
| | | ASRERTSRYFD | KSMNYPNSAS | HSQKVGAAI | YPSQEFLDV | | |
| | | DYQLTSKRTC | IGNLCGSLAG | QLIDDWWD | KESGKPTK | | |
| | | DVLAHLAGFE | HINVLNYPVD | EEADKPLRV | QLAKVVLN | | |
| | | QLKSRKAQKH | VVPDSYQTLA | NEYGADKEY | GEEESAAY | | |
| | | VRQYQLKIIRK | ASRERTSRYFD | VIARRHSSLK | HSQKVGAA | | |
| | | QIARWLLPLIE | DYQLTSKRTC | RDFYSLISKTE | IQLIDDWW | | |
| | | LRDNLVTEPL | DVLAHLAGFE | DHIESMRKS | DEEADKPL | | |
| | | GINYEFDDQL | QLKSRKAQKH | NDISNDIHFI | RVNEYGAD | | |
| | | AKQFLTIKEDD | VRQYQLKIIRK | MAVLAKGG | KEYVIARRH | | |
| | | FLDWTTSLNQ | QIARWLLPLIE | VFSGASKKSK | SSLKRDFYS | | |
| | | RLNLALQNNR | LRDNLVTEPL | KEE (SEQ ID | LISKTEDHIE | | |
| | | FSSRFAYHPKL | GINYEFDDQL | NO: 459) | SMRKSNDI | | |
| | | MRVLKTELIW | AKQFLTIKEDD | | SNDIHFIMA | | |
| | | VLTQLSRPEP | FLDWTTSLNQ | | VLAKGGVF | | |
| | | GLPNISNDSV | RLNLALQNNR | | SGASKKSKK | | |
| | | QYIYLSSMRA | FSSRFAYHPKL | | EE (SEQ ID | | |
| | | FDVAALSCPYL | MRVLKTELIW | | NO: 460) | | |
| | | SGAPSMTAI | VLTQLSRPEP | | | | |
| | | WGFIHRYQKE | GLPNISNDSV | | | | |
| | | LEAQMSDEQ | QYIYLSSMRA | | | | |
| | | CRISFNEFAFFI | FDVAALSCPYL | | | | |
| | | RHESVQTSAK | SGAPSMTAI | | | | |
| | | LTEPSVLAKAR | WGFIHRYQKE | | | | |
| | | EVSPVKRTTII | LEAQMSDEQ | | | | |
| | | REDYADLVFD | CRISFNEFAFFI | | | | |
| | | LVIRVESNQRI | RHESVQTSAK | | | | |
| | | SDYHDQLKAA | LTEPSVLAKAR | | | | |
| | | LPTNFAGGTL | EVSPVKRTTII | | | | |
| | | LQPEIDLNIP | REDYADLVFD | | | | |
| | | WLRTYTTKSE | LVIRVESNQRI | | | | |
| | | LFQVVKGLPG | SDYHDQLKAA | | | | |
| | | YGTWLSPYSY | LPTNFAGGTL | | | | |
| | | QPQNLTELEN | LQPEIDLNIP | | | | |
| | | TLAKDASLIPIV | WLRTYTTKSE | | | | |
| | | NGFHLLEKPIN | LFQVVKGLPG | | | | |
| | | RKNGLTNRHA | YGTWLSPYSY | | | | |
| | | YAENNIALAK | QPQNLTELEN | | | | |
| | | RVNPIEVRFG | TLAKDASLIPIV | | | | |
| | | GRDHFFEQAF | NGFHLLEKPIN | | | | |
| | | WSLDVTEQTI | RKNGLTNRHA | | | | |
| | | LIKNLRN (SEQ | YAENNIALAK | | | | |
| | | ID NO: 457) | RVNPIEVRFG | | | | |
| | | | GRDHFFEQAF | | | | |
| | | | WSLDVTEQTI | | | | |
| | | | LIKNLRN (SEQ | | | | |
| | | | ID NO: 458) | | | | |
| 42 | V.natriegens | MTTLQDLIDIE | MPKKKRKVGS | MELCSQLNY | MPKKKRKV | MGSRCYFSI | MPKKKRKV |
| | strain | DSKLRFIAIKK | GDYKDDDDK | LRSLSPGKAY | GSGELCSQL | RYVPDYADN | GSGGSRCY |
| | CCUG | AFMPYTQPVE | DYKDDDDKD | FYYLDEDNK | NYLRSLSPG | ELLAGRCISN | FSIRYVPDY |
| | 16373 | IDGNEKQALIV | YKDDDDKGS | MRPLQIDRT | KAYFYYLDE | MHGFLSHER | ADNELLAG |
| | | LINLSLSKPEA | GTTLQDLIDIE | HLRAPKSGY | DNKMRPL | NKPFKNSVGI | RCISNMHG |
| | | QDWLDLSRA | DSKLRFIAIKK | SEAFSGNFKS | QIDRTHLRA | CFPVWNEQ | FLSHERNKP |
| | | MGYFANSDN | AFMPYTQPVE | KNIAPQDLSY | PKSGYSEAF | TVGNVITFVS | FKNSVGICF |
| | | LTTAKREIQW | IDGNEKQALIV | SNPQFIEECY | SGNFKSKNI | TNESILTGLS | PVWNEQT |
| | | FHTHNLKFPD | LINLSLSKPEA | VPPGVDDIY | APQDLSYS | YQPYFSRMV | VGNVITFVS |
| | | CRVSEQRIIA | QDWLDLSRA | CAFSLRVRA | NPQFIEECY | NENLFEISDI | TNESILTGLS |
| | | MPLYSETPTLT | MGYFANSDN | NSLSPEVCV | VPPGVDDIY | KAVPDDAEE | YQPYFSRM |
| | | SQSLNRVYG | LTTAKREIQW | DNEVRDILC | CAFSLRVRA | VRFVFNKTIQ | VNENLFEIS |
| | | WAHNSTVYK | FHTHNLKFPD | NFAALYKEL | NSLSPEVCV | KIFNGSKKRR | DIKAVPDD |
| | | HTIWLLNEFR | CRVSEQRIIA | GGYRELARR | DNEVRDILC | IKRAMKRAE | AEEVRFVF |
| | | WRGRVENLL | MPLYSETPTLT | YAQNILMAT | NFAALYKEL | EFGHAFTPIS | NKTIQKIFN |
| | | NLIRVGEHFW | SQSLNRVYG | WVWRNREC | GGYRELAR | VEEREFELFH | GSKKRRIKR |
| | | LELLADIGLKP | WAHNSTVYK | RSIRVEVKTE | RYAQNILM | EIPISSKSSGH | AMKRAEEF |
| | | EVQLQIKELIE | HTIWLLNEFR | DKEWVITDA | ATWVWRN | DFVLHIQRQ | GHAFTPISV |
| | | RQLPSTHFPD | WRGRVENLL | RFLDWYGS | RECRSIRVE | YPVVAEIEQ | EEREFELFH |
| | | EVNRYSKQLR | NLIRVGEHFW | WDKDSQLAL | VKTEDKEW | HFNGYGFAS | EIPISSKSSG |
| | | FPWKDEYLSV | LELLADIGLKP | DEFTGYLSQ | VITDARFLD | NQLWQGTV | HDFVLHIQ |
| | | TPVVSHAIQQ | EVQLQIKELIE | ALSDRTSYFN | WYGSWDK | PLISF (SEQ | RQYPVVAEI |
| | | QLSVLSRQHS | RQLPSTHFPD | MDIKAKLTV | DSQLALDEF | ID NO: 467) | EQHFNGYG |
| | | CSFHFKTMNF | EVNRYSKQLR | GWGDEVYP | TGYLSQALS | | FASNQLW |
| | | PHSASIGNLC | FPWKDEYLSV | SQEFLDVKE | DRTSYFNM | | QGTVPLISF |
| | | GSLGGNMDIL | TPVVSHAIQQ | AGKPTKQLA | DIKAKLTVG | | (SEQ ID |
| | | NYPIGVIANR | QLSVLSRQHS | KVLVNGAES | WGDEVYPS | | NO: 468) |
| | | HQTLGASRSR | CSFHFKTMNF | AAFHSQKIG | QEFLDVKE | | |
| | | TNRYFDDFQL | PHSASIGNLC | AAIQLIDDW | AGKPTKQL | | |
| | | TSKRTCGVLA | GSLGGNMDIL | WDENADKP | AKVLVNGA | | |
| | | HLTGFEQPQ | NYPIGVIANR | LRVNEYGAD | ESAAFHSQ | | |
| | | MRKAQKHVR | HQTLGASRSR | KEYVIARRHS | KIGAAIQLI | | |
| | | QYQLKIIRRQI | TNRYFDDFQL | SLKRDFYSLA | DDWWDEN | | |
| | | ALWLLPLIELR | TSKRTCGVLA | AKTESYVES | ADKPLRVN | | |
| | | DNLVTEPIGFY | HLTGFEQPQ | MRETNLIPD | EYGADKEY | | |
| | | DESDDELAKR | MRKAQKHVR | DVHFIMAVL | VIARRHSSL | | |
| | | FLTINELDFIVL | QYQLKIIRRQI | TKGGVFSGA | KRDFYSLAA | | |
| | | TTSLNQRLNL | ALWLLPLIELR | SKKGKKDE | KTESYVES | | |
| | | ALQNNRFASR | DNLVTEPIGFY | (SEQ ID | MRETNLIP | | |
| | | FAYHPKLMRV | DESDDELAKR | NO: 465) | DDVHFIMA | | |
| | | LKTELIWVLTQ | FLTINELDFIVL | | VLTKGGVFS | | |
| | | LSRPEPACSAT | TTSLNQRLNL | | GASKKGKK | | |
| | | SDSTVQYLYLP | ALQNNRFASR | | DE (SEQ ID | | |
| | | SMRVFDAAAL | FAYHPKLMRV | | NO: 466) | | |
| | | SCPYLSGAPSL | LKTELIWVLTQ | | | | |
| | | TAVFGFVHRY | LSRPEPACSAT | | | | |
| | | QRELRDLLPD | SDSTVQYLYLP | | | | |
| | | KEGKLKFKDF | SMRVFDAAAL | | | | |
| | | AIFIRDESVQT | SCPYLSGAPSL | | | | |
| | | SAKLTEPSVIA | TAVFGFVHRY | | | | |
| | | KARGISPVKRT | QRELRDLLPD | | | | |
| | | TIIREDCSDLV | KEGKLKFKDF | | | | |
| | | FDIVITIESDQR | AIFIRDESVQT | | | | |
| | | LSDYLNQLRA | SAKLTEPSVIA | | | | |
| | | ALPTNFAGGT | KARGISPVKRT | | | | |
| | | LLQPETSLGID | TIIREDCSDLV | | | | |
| | | WLSIFVSESDL | FDIVITIESDQR | | | | |
| | | FQAVKGLPGY | LSDYLNQLRA | | | | |
| | | GTWLSPYSFQ | ALPTNFAGGT | | | | |
| | | PQNLMELQE | LLQPETSLGID | | | | |
| | | RLSNDGSLIPV | WLSIFVSESDL | | | | |
| | | ANGFHFLELP | FQAVKGLPGY | | | | |
| | | QEREGALTNL | GTWLSPYSFQ | | | | |
| | | HCYAENNIAL | PQNLMELQE | | | | |
| | | AKRVSPIEVRI | RLSNDGSLIPV | | | | |
| | | AGRDHFFEQV | ANGFHFLELP | | | | |
| | | FWSLEVTEQT | QEREGALTNL | | | | |
| | | ILIKKGSNRLW | HCYAENNIAL | | | | |
| | | NSAVS (SEQ | AKRVSPIEVRI | | | | |
| | | ID NO: 463) | AGRDHFFEQV | | | | |
| | | | FWSLEVTEQT | | | | |
| | | | ILIKKGSNRLW | | | | |
| | | | NSAVS (SEQ | | | | |
| | | | ID NO: 464) | | | | |

In one aspect the disclosure includes a kit comprising one or more expression vector(s) that encodes one or more Cas or other enzymes described herein. The expression vector in certain approaches includes a cloning site, such as a poly-cloning site, such that any desirable cargo gene(s) can be cloned into the cloning site to be expressed in any target cell into which the system is introduced or already comprises. The kit can further comprise one or more containers, printed material providing instructions as to how to use make and/or use the expression vector to produce suitable vectors, and reagents for introducing the expression vector into cells. The kits may further comprise one or more bacterial strains for use in producing the components of the system. The bacterial strains may be provided in a composition wherein growth of the bacteria is restricted, such as a frozen culture with one or more cryoprotectants, such as glycerol. In embodiments, the kit comprises a vector for expression of a guide RNA comprising a user selected spacer.

In another aspect the disclosure comprises delivering to cells a DNA cargo via a system of this disclosure. The method generally comprises introducing one or more polynucleotides of this disclosure, or a mixture or proteins and polynucleotides encoding the proteins, which may be also provided with RNA polynucleotides, such as the presently described guide RNAs, into one or more bacterial or eukaryotic cells, whereby the Cas and transposon enzymes/proteins are expressed and editing of the chromosome or another DNA target by a combination of the Cas enzymes and the transposon occurs.

In non-limiting embodiments, this disclosure is considered to be suitable for targeting eukaryotic cells, and any microorganism that is susceptible to editing by a system as described herein. In embodiments the microorganism comprises bacteria that are resistant to one or more antibiotics, whereby the editing by the present system kills or reduces the growth of the antibiotic-resistant bacteria, and/or the system sensitizes the bacteria to an antibiotic by, for example, use of cargo that targets an antibiotic resistance gene, which may be present on a chromosome or a plasmid. The disclosure is thus suitable for targeting bacterial chromosomes or episomal elements, e.g., plasmids. In embodiments, a modification of a bacterial chromosome or plasmid causes the bacteria to change from pathogenic to non-pathogenic.

In embodiments, bacteria are killed. In embodiments, one or all of the components of a system described herein can be provided in a pharmaceutical formulation. Thus, in embodiments, DNA, RNA, proteins, and combinations thereof can be provided in a composition that comprises at least one pharmaceutically acceptable additive.

In embodiments, the method of this disclosure is used to reduce or eradicate bacterial cells, and may be used to reduce or eradicate persister bacteria and/or dormant viable but non-culturable (VBNC) bacteria from an individual or an inanimate surface, or a food substance.

In embodiments, and as noted above, the disclosure is considered suitable for editing eukaryotic cells. In embodiments, eukaryotic cells that are modified by the approaches of this disclosure are totipotent, pluripotent, multipotent, or oligopotent stem cells when the modification is made. In embodiments, the cells are neural stem cells. In embodiments, the cells are hematopoietic stem cells. In embodiments, the cells are leukocytes. In embodiments, the leukocytes are of a myeloid or lymphoid lineage. In embodiments, the cells are embryonic stem cells, or adult stem cells. In embodiments, the cells are epidermal stem cells or epithelial stem cells. In embodiments, the cells are cancer cells, or cancer stem cells. In embodiments, the cells are differentiated cells when the modification is made. In embodiments, the cells are mammalian cells. In embodiments, the cells are human, or are non-human animal cells. In embodiments, the non-human eukaryotic cells comprise fungal, plant or insect cells. In one approach the cells are engineered to express a detectable or selectable marker, or a combination thereof.

In embodiments, the disclosure includes obtaining cells from an individual, modifying the cells ex vivo using a CRISPR system as described herein, and reintroducing the cells or their progeny into the individual for prophylaxis and/or therapy of a condition, disease or disorder, or to treat an injury, trauma or anatomical defect. In embodiments, the cells modified ex vivo as described herein are used autologously.

In embodiments, cells modified according to this disclosure are provided as cell lines. In embodiments, the cells are engineered to produce a protein or other compound, and the cells themselves or the protein or compound they produce is used for prophylactic or therapeutic applications.

In various embodiments, the modification introduced into eukaryotic cells according to this disclosure is homozygous or heterozygous. In embodiments, the modification comprises a homozygous dominant or homozygous recessive or heterozygous dominant or heterozygous recessive mutation correlated with a phenotype or condition, and is thus useful for modeling such phenotype or condition. In embodiments a modification causes a malignant cell to revert to a non-malignant phenotype.

In certain aspects the disclosure includes a pharmaceutical formulation comprising one or more components of a system described herein. A pharmaceutical formulation comprises one or more pharmaceutically acceptable additives, many of which are known in the art. In some embodiments, the pharmaceutical compositions comprise a pharmaceutically acceptable carrier suitable for administration to humans. In some embodiments, the pharmaceutical compositions comprise a pharmaceutically acceptable carrier suitable for intraocular injection. In some embodiments, the pharmaceutical compositions comprise a pharmaceutically acceptable carrier suitable for topical application. In some embodiments, the pharmaceutical compositions comprise a pharmaceutically acceptable carrier suitable for intravenous injection. In some embodiments, the pharmaceutical compositions comprise and a pharmaceutically acceptable carrier suitable for injection into arteries. In some embodiments, the pharmaceutical composition is suitable for oral or topical administration. All of the described routes of administration are encompassed by the disclosure.

In embodiments, expression vectors, proteins, RNPs, polynucleotides, and combinations thereof, can be provided as pharmaceutical formulations. A pharmaceutical formulation can be prepared by mixing the described components with any suitable pharmaceutical additive, buffer, and the like. Examples of pharmaceutically acceptable carriers, excipients and stabilizers can be found, for example, in Remington: The Science and Practice of Pharmacy (2005) 21st Edition, Philadelphia, PA. Lippincott Williams & Wilkins, the disclosure of which is incorporated herein by reference. Further, any of a variety of therapeutic delivery agents can be used, and include but are not limited to nanoparticles, lipid nanoparticle (LNP), exosomes, and the like. In embodiments, a biodegradable material can be used. In embodiments, poly(lactide-co-galactide) (PLGA) is a representative biodegradable material. In embodiments, any biodegradable material, including but not necessarily limited to biodegrable polymers. As an alternative to PLGA, the biodegradable material can comprise poly(glycolide) (PGA), poly(L-lactide) (PLA), or poly(beta-amino esters). In embodiments, the biodegradable material may be a hydrogel, an alginate, or a collagen. In an embodiment the biodegradable material can comprise a polyester a polyamide, or polyethylene glycol (PEG). In embodiments, lipid-stabilized micro and nanoparticles can be used.

In certain approaches, compositions of this disclosure, including the described systems, and cells modified using the described systems, are used for treatment of condition or disorder in an individual in need thereof. The term “treatment” as used herein refers to alleviation of one or more symptoms or features associated with the presence of the particular condition or suspected condition being treated. Treatment does not necessarily mean complete cure or remission, nor does it preclude recurrence or relapses. Treatment can be effected over a short term, over a medium term, or can be a long-term treatment, such as, within the context of a maintenance therapy. Treatment can be continuous or intermittent.

In embodiments, a system of this disclosure is administered to an individual in a therapeutically effective amount. In embodiments, a therapeutically effective amount of a composition of this disclosure is used. The term “therapeutically effective amount” as used herein refers to an amount of an agent sufficient to achieve, in a single or multiple doses, the intended purpose of treatment. The amount desired or required will vary depending on the particular compound or composition used, its mode of administration, patient specifics and the like. Appropriate effective amounts can be determined by one of ordinary skill in the art informed by the instant disclosure using routine experimentation. For example, a therapeutically effective amount, e.g., a dose, can be estimated initially either in cell culture assays or in animal models. An animal model can also be used to determine a suitable concentration range, and route of administration. Such information can then be used to determine useful doses and routes for administration in humans, or to non-human animals. A precise dosage can be selected by in view of the patient to be treated. Dosage and administration can be adjusted to provide sufficient levels of components to achieve a desired effect, such as a modification in a threshold number of cells. Additional factors which may be taken into account include the particular gene or other genetic element involved, the type of condition, the age, weight and gender of the patient, desired duration of treatment, method of administration, time and frequency of administration, drug combination(s), reaction sensitivities, and tolerance/response to therapy. In certain embodiments, a therapeutically effective amount is an amount that reduces one or more signs or symptoms of a disease, and/or reduces the severity of the disease. A therapeutically effective amount may also inhibit or prevent the onset of a disease, or a disease relapse. In embodiments, cells modified according to this disclosure are administered to an individual in need thereof in a therapeutically effective amount.

In embodiments, the disclosure comprises providing a treatment to an individual in need thereof by introducing a therapeutically effective amount a composition of this disclosure, or modified cells as described herein to the individual, wherein the cells comprising the DNA insertion treats, alleviates, inhibits, or prevents the formation of one or more conditions, diseases, or disorders. In embodiments, the cells are first obtained from the individual, modified according to this disclosure, and transplanted back into the individual. In embodiments, allogenic cells can be used. In embodiments, the modified eukaryotic cells can be provided in a pharmaceutical formulation, and such formulations are included in the disclosure.

In embodiments, a described system of this disclosure is introduced into one or more prokaryotic or eukaryotic cells. In embodiments, the prokaryotic cells comprise or consist of gram positive, or gram negative bacteria. The bacteria may be non-pathogenic, or pathogenic. In embodiments, a described system is introduced into prokaryotic cells (e.g., bacterial or archaeal cells) in the context of a host, e.g., a human, animal, or plant host, e.g., the bacteria are a component of a host's microbiome or are an abnormal component of a microbiome, e.g., a pathogen. In some embodiments, delivery of a system described herein results in the stable formation of a recombinant microorganism. In some embodiments, a recombinant microorganism as generated by a system described herein results in the production of an enzyme or metabolite that can alter the health or metabolism of a host, e.g., a human host. In some embodiments, delivery of a system described herein results in the inactivation of virulence determinants of a microorganism, e.g., antibiotic resistance or toxin production. In some embodiments, delivery of a system described herein results in killing of the recipient cell. The system may kill some or all of the cells, or render the cells non-pathogenic and/or sensitive to one or more antibiotics. In embodiments, the bacteria are used as a component of a food or beverage product, including but not limited to fermented food and beverages, and dairy products. In embodiments, such bacteria comprise Lactic acid bacteria. In embodiments, selective delivery to a specific type of bacteria is used by way of a bacteriophage or packaged phagemids that can express all or some of the described components, but wherein the bacteriophage exhibits a specific tropism for a particular type of bacteria. In some embodiments, a delivery vehicle provides only partial specificity towards targeting particular cells, and additional specificity is provided by the choice of DNA sequence being targeted.

In embodiments, the described systems are introduced into eukaryotic cells. Such cells include but are not necessarily limited to animal cells, fungi such as yeasts, protists, algae, and plant cells.

In embodiments, the disclosure provides one or more cells, wherein DNA in the cells comprises at least one inserted DNA insertion template. The described cells may be any prokaryotic or eukaryotic cells. Accordingly, the disclosure also provides one or more cells that comprise an inserted DNA sequence.

In embodiments, the eukaryotic cells comprise animal cells, which may comprise mammalian or avian cells, or insect cells. In embodiments, the mammalian cells are human or non-human mammalian cells. In embodiments, compositions of this disclosure are administered to avian animals, or to a canine, a feline, an equine animal, or to cattle, including but not limited to dairy cattle.

In embodiments, the cells that are modified by the approaches of this disclosure are totipotent, pluripotent, multipotent, or oligopotent stem cells when the modification is made. In embodiments, the cells are neural stem cells. In embodiments, the cells are hematopoietic stem cells. In embodiments, the cells are leukocytes. In embodiments, the leukocytes are of a myeloid or lymphoid lineage. In embodiments, the cells are embryonic stem cells, or adult stem cells. In embodiments, the cells are epidermal stem cells or epithelial stem cells. In embodiments, the cells are cancer cells, or cancer stem cells. In embodiments, the cells are differentiated cells when the modification is made.

In embodiments, the disclosure includes obtaining cells from an individual, modifying the cells ex vivo using a system as described herein, and reintroducing the cells or their progeny into the individual or a immunologically matched individual for prophylaxis and/or therapy of a condition, disease or disorder, or to treat an injury, trauma or anatomical defect. In embodiments, the cells modified ex vivo as described herein are autologous cells. In embodiments, the cells are provided as cell lines. In embodiments, the cells are engineered to produce a protein or other compound, and the cells themselves and/or the protein or compound they produce is used for prophylactic or therapeutic applications.

In embodiments, eukaryotic cells made according to this disclosure can be used to create transgenic, non-human organisms.

In embodiments, one or more modified cells according to this disclosure may be used to perform a gene-drive in a population of animals, including but not necessarily limited to insects.

In embodiments, the one or more cells into which a described system is introduced comprises a plant cell. The term “plant cell” as used herein refers to protoplasts, gamete producing cells, and includes cells which regenerate into whole plants. Plant cells include but are not necessarily limited to cells obtained from or found in: seeds, suspension cultures, embryos, meristematic regions, callus tissue, leaves, roots, shoots, gametophytes, sporophytes, pollen, and microspores. Plant cells can also be understood to include modified cells, such as protoplasts, obtained from the aforementioned tissues. Plant products made according to the disclosure are included.

In embodiments, the disclosure provides an article of manufacture, which may comprise a kit. In embodiments, the article of manufacture may comprise one or more cloning vectors. The one or more cloning vectors may encode any one or combination of proteins and polynucleotides described herein. The cloning vectors may be adapted to include, for example, a multiple cloning site (MCS), into which a sequence encoding any protein or polynucleotide, such as any desired targeting RNA, may be introduced. An article of manufacture may include one or more sealed containers that contain any of the aforementioned components, and may further comprise packaging and/or printed material. The printed material may provide information on the contents of the article, and may provide instructions or other indication of how the contents of the article may be used. In an embodiment, the printed material provides an indication of a disease or disorder that is to be treated using the contents of the article.

In embodiments, when polynucleotides are delivered, they may comprise modified polynucleotides or other modifications, such as phosphate backbone modifications, and modified nucleotides, such as nucleotide analogs. Suitable modifications and methods for making nucleic acid analogs are known in the art. Some examples include but are not limited to polynucleotides which comprise modified ribonucleotides or deoxyribonucleotides. For example, modified ribonucleotides may comprise methylations and/or substitutions of the 2′ position of the ribose moiety with an —O— lower alkyl group containing 1-6 saturated or unsaturated carbon atoms, or with an —O-aryl group having 2-6 carbon atoms, wherein such alkyl or aryl group may be unsubstituted or may be substituted, e.g., with halo, hydroxy, trifluoromethyl, cyano, nitro, acyl, acyloxy, alkoxy, carboxyl, carbalkoxy, or amino groups; or with a hydroxy, an amino or a halo group. In embodiments modified nucleotides comprise methyl-cytidine and/or pseudo-uridine. The nucleotides may be linked by phosphodiester linkages or by a synthetic linkage, i.e., a linkage other than a phosphodiester linkage. Examples of inter-nucleoside linkages in the polynucleotide agents that can be used in the disclosure include, but are not limited to, phosphodiester, alkylphosphonate, phosphorothioate, phosphorodithioate, phosphate ester, alkylphosphonothioate, phosphoramidate, carbamate, carbonate, morpholino, phosphate triester, acetamidate, carboxymethyl ester, or combinations thereof. In embodiments, the DNA analog may be a peptide nucleic acid (PNA).

The Examples of this disclosure are illustrated by the accompanying figures. While the disclosure has been described in conjunction with the detailed description and the Figures, this description is intended to illustrate and not limit the scope of the invention.

What is claimed is:

1. One or more modified I-F3 proteins for use in a Clustered Regularly Interspaced Short Palindromic Repeats (CRISPR) system to modify a DNA substrate, wherein the one or more proteins are selected from:
i) a TnsC protein comprising an insertion of one or more amino acids;
ii) a TnsA protein comprising an insertion of one or more amino acids;
iii) a TnsB protein comprising an insertion of one or more amino acids; and
iv) a single protein comprising the amino acid sequence of a TnsA protein and the amino acid sequence of a TnsB protein, wherein optionally the TnsA protein, the TnsB protein, or both, comprise an insertion between the amino acid sequences of the TnsA and TnsB proteins.

2. The one or more modified I-F3 proteins of claim 1 wherein the CRISPR system comprising the one or more modified I-F3 proteins is capable of exhibiting a higher transposition frequency relative to an I-F3 system comprising the same I-F3 proteins in unmodified form.

3. The one or more modified I-F3 proteins of claim 1, wherein the insertion of the one or more amino acids is between the N and C termini of the one or more modified proteins.

4. The one or more modified I-F3 proteins of claim 1, wherein the CRISPR system further comprises an I-F3 TniQ protein, and optionally a guide RNA targeted to a location in a chromosome or plasmid, and optionally a double stranded DNA template for introduction into the chromosome or plasmid targeted by the guide RNA.

5. The one or more modified I-F3 proteins of claim 1, wherein the insertion is an insertion of 2-30 amino acids, and wherein the insertion optionally comprises a nuclear localization sequence or a protein purification sequence.

6. The one or more modified I-F3 proteins of claim 1, wherein the modified protein is a modified TnsC protein, wherein the insertion is C-terminal to amino acid 144 of a wild type TnsC protein or at a corresponding position in a homologous or orthologous protein.

7. The one or more modified I-F3 proteins of claim 1, wherein the modified protein is a modified TnsC protein, wherein the insertion is N-terminal to amino acid 144 of a wild type TnsC protein or at a corresponding position in a homologous or orthologous protein.

8. The one or more modified I-F3 proteins of claim 1, wherein the modified protein is a modified TnsC protein, and wherein the insertion is between amino acid 144 and 150 of a wild type TnsC protein or at a corresponding position in a homologous or orthologous protein.

9. The one or more modified I-F3 proteins of claim 1, wherein the modified protein is a modified TnsC protein, wherein the insertion is C-terminal to amino acid 304 of a wild type TnsC protein or at a corresponding position in a homologous or orthologous protein.

10. The one or more modified I-F3 proteins of claim 1, wherein the modified protein is a modified TnsC protein, wherein the insertion is N-terminal to amino acid 304 of a wild type TnsC protein or at a corresponding position in a homologous or orthologous protein.

11. The one or more modified I-F3 proteins of claim 1, wherein the modified protein is a modified TnsC protein, and wherein the insertion is between amino acid 300 and 310 of a wild type TnsC protein or at a corresponding position in a homologous or orthologous protein.

12. The one or more modified I-F3 proteins of claim 1, wherein the modified protein comprises the amino acid sequence of a TnsA protein and the amino acid sequence of a TnsB protein and an insertion between the TnsA protein and the TnsB protein.

13. A Clustered Regularly Interspaced Short Palindromic Repeats (CRISPR) system comprising the one or more modified I-F3 proteins of any one of claims 1-12.

14. The CRISPR system of claim 13, further comprising an I-F3 TniQ protein.

15. The CRISPR system of claim 13, further comprising a guide RNA targeted to a location in a chromosome or plasmid, and optionally a double stranded DNA template for introduction into a chromosome or plasmid targeted by the guide RNA.

16. The CRISPR system of claim 13, further comprising Cas8, Cas5, Cas7, and Cas6 proteins.

17. A method comprising introducing into cells a CRISPR system of claim 13 and a guide RNA targeted to a location in a chromosome or plasmid, or one or more polynucleotides encoding one or more of the modified proteins and/or the guide RNA.

18. The method of claim 17, wherein the CRISPR system further comprises an I-F3 TniQ protein or polynucleotide encoding the TniQ protein.

19. The method of claim 17, wherein the CRISPR system further comprises Cas8, Cas5, Cas7, and Cas6 proteins, or a polynucleotide encoding one or more of the Cas8, Cas5, Cas7, and Cas6 proteins.

20. The method of claim 17, wherein a chromosome or plasmid within the cells is modified by the CRISPR system and the guide RNA at a location that is linked to the location that is targeted by the guide RNA.

21. The method of claim 18, wherein a chromosome or plasmid within the cells is modified by the CRISPR system and the guide RNA at a location that is linked to the location that is targeted by the guide RNA.

22. The method of claim 19, wherein a chromosome or plasmid within the cells is modified by the CRISPR system and the guide RNA at a location that is linked to the location that is targeted by the guide RNA.

23. The method of claim 20, wherein frequency of modification of the location that is linked to the location that is targeted by the guide RNA occurs more frequently in the cells relative to a value for frequency of modification of the same target using the same guide RNA and the same proteins but without protein modifications.

24. The method of claim 21, wherein frequency of modification of the location that is linked to the location that is targeted by the guide RNA occurs more frequently in the cells relative to a value for frequency of modification of the same target using the same guide RNA and the same proteins but without protein modifications.

25. The method of claim 22, wherein frequency of modification of the location that is linked to the location that is targeted by the guide RNA occurs more frequently in the cells relative to a value for frequency of modification of the same target using the same guide RNA and the same proteins but without protein modifications.

26. A polynucleotide encoding at least one of the modified I-F3 proteins of any one of claims 1-12.

27. The polynucleotide of claim 26, further encoding a guide RNA.

28. A modified cell comprising a modified I-F3 protein of any one of claims 1-12.
